(19) United States
(12) Patent Application Publication
(10) Pub. No.: US 2023/0174484 A1
(43) Pub. Date: Jun. 8, 2023

(54) ANTAGONISTS OF GPR39 PROTEIN

(71) Applicant: Oregon Health & Science University, Portland, OR (US)

(72) Inventors: Sanjiv KAUL, Portland, OR (US); Nabil ALKAYED, Portland, OR (US); Shanthi NAGARAJAN, Portland, OR (US); Agostino CIANCIULLI, Verona (IT); Fabrizio MICHELI, Verona (IT); Teresa SEMERARO, Verona (IT); Iuni Margaret Laura TRIST, Verona (IT)

(21) Appl. No.: 17/997,533

(22) PCT Filed: Apr. 30, 2021
(86) PCT No.: PCT/US21/30335

§ 371 Date: Oct. 28, 2022

Related U.S. Application Data
(60) Provisional application No. 63/018,371, filed on Apr. 30, 2020.

(51) Int. Cl. C07D 209/54; C07D 207/06; C07D 267/10; C07D 491/107; C07D 211/38; C07D 231/12; C07D 207/327; C07D 295/192; C07D 213/56; C07D 209/52; C07D 211/14; C07D 217/04; C07D 223/04; C07D 261/08; C07D 263/32; C07D 213/36; C07D 495/04; C07D 217/06; C07D 487/04; C07D 213/40; C07D 295/155; C07D 213/74; C07D 401/04; C07D 417/10; C07D 401/10; C07D 403/10; C07D 409/14; C07D 403/04; C07D 409/04; C07D 241/04; C07D 413/06; C07D 249/14; C07D 241/26; C07D 213/81; C07D 249/12; C07D 249/08; C07D 401/12; C07F 5/02; C07D 413/10; C07D 401/14; C07D 265/30; C07D 279/12; C07D 491/08; C07D 221/22; C07D 265/34; A61P 9/12; A61P 9/10

(52) U.S. Cl. CPC C07D 209/54; C07D 207/06; C07D 267/10; C07D 491/107; C07D 211/38; C07D 231/12; C07D 207/327; C07D 295/192; C07D 213/56; C07D 209/52; C07D 211/14; C07D 217/04; C07D 223/04; C07D 261/08; C07D 263/32; C07D 213/36; C07D 495/04; C07D 217/06; C07D 487/04; C07D 213/40; C07D 295/155; C07D 213/74; C07D 401/04; C07D 417/10; C07D 401/10; C07D 403/10; C07D 409/14; C07D 403/04; C07D 409/04; C07D 241/04; C07D 413/06; C07D 249/14; C07D 241/26; C07D 213/81; C07D 249/12; C07D 249/08; C07D 401/12; C07F 5/025; C07D 413/10; C07D 401/14; C07D 265/30; C07D 279/12; C07D 491/08; C07D 221/22; C07D 265/34; A61P 9/12; A61P 9/10

(57) ABSTRACT

Novel compounds that act as antagonists to human GPR39 protein are disclosed. Pharmaceutical compositions and methods of use for antagonists to human GPR39 protein are disclosed. In particular, methods of using the antagonists in the treatment of diseases or conditions including cardiovascular conditions, endocrine system and hormone disorders, cancer disorders, metabolic diseases, gastrointestinal and liver diseases, hematological disorders, neurological disorders and respiratory diseases are disclosed herein.

### RELATED APPLICATIONS

This application is a National Stage of International Application No. PCT/US2021/030335, filed on Apr. 30, 2021, which claims priority to United States Provisional Patent Application No. 63/018,371, filed on Apr. 30, 2020, entitled “ANTAGONISTS OF GPR39 PROTEIN,” which are hereby incorporated by reference in their entireties.

### FIELD OF THE INVENTION

The present disclosure concerns novel compounds that act as antagonists to human GPR39 protein. Additionally, the present disclosure relates to pharmaceutical compositions and methods of using antagonists to human GPR39 protein in the treatment of diseases or conditions including cardiovascular conditions, endocrine system and hormone disorders, cancer disorders, metabolic diseases, gastrointestinal and liver diseases, hematological disorders, neurological disorders and respiratory diseases.

### BACKGROUND OF THE INVENTION

Use of GPR39 antagonists has been described for methods of treating pain sensitivity, including hyperalgesia, and suppressing appetite (U.S. Pat. Publication 2009/0298756—Jin et al.).

The use of GPR39 agonists and/or antagonists in enhancing glucose regulation and treating impaired carbohydrate metabolism, including in disorders such as diabetes and metabolic syndrome are discussed in WO 2007/141322—Moreaux et al.—Janssen Pharmaceutica N.V.

The article Altered Gastrointestinal and Metabolic Function in the GPR39—Obestatin Receptor—Knockout Mouse, Gastroenterology, Moechars et al., October 2006, Vol. 131, Issue 4, pp. 1131-1141, discloses conclusions that GPR39 receptor antagonists may be useful in disorders affecting stomach motility, including such as functional dyspepsia and diabetic gastroparesis and/or colorectal motility such as irritable bowel syndrome, diarrhea, or chronic constipation.

The role of the obestatin/GPR39 system in human gastric adenocarcinomas, Alén et al., Oncotarget 2016, 7:5957-5971 addresses the role of the obestatin/GPR39 system in regulating motility, EMT, and invasion of gastric adenocarcinoma cells.

GPR39 antagonists are also discussed in the article Enhanced ZnR/GPR39 Activity in Breast Cancer, an Alternative Trigger of Signaling Leading to Cell Growth, Ventura-Bixenshpaner et al., Scientific Reports (2018) 8:8119, particularly their use in treating breast cancer, including ER negative breast cancer.

The use of GPR39 antagonists in the treatment of various cancers is discussed in U.S. 2004/0071708 (Claassen et al.).

Inge Depoortere discloses uses of GPR39 antagonists in treating motility disorders, such as functional dyspepsia, hypoparesis, and chronic constipation in her article, GI functions of GPR39: novel biology, Current Opinion in Pharmacology, 2012, 12:647-652.

The article The zinc sensing receptor, ZnR/GPR39, controls proliferation and differentiation of colonocytes and thereby tight junction formation in the colon, Cohen et al., Cell Death and Disease (2014) 5, e1307, discusses potential uses for GPR antagonists in promoting or enhancing colon epithelial function and tight junction barrier integrity, including treating ulcerative colon diseases, such as ulcerative colitis, and diarrheal pathologies.

### BRIEF SUMMARY OF THE INVENTION

Provided is a compound of Formula (I), or a pharmaceutically acceptable salt thereof:

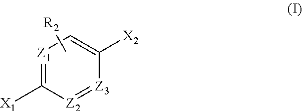

wherein:

X₁ is selected from the group of:

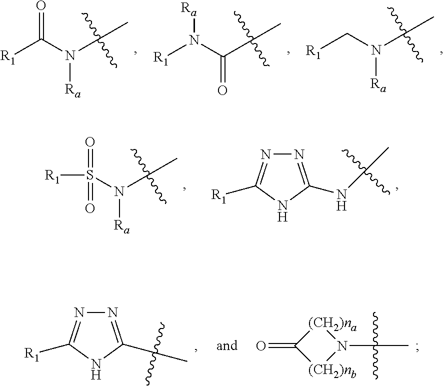

nₐ is an integer selected from the group of 0, 1, and 2;

n_{b} is an integer selected from the group of 0, 1, 2, 3, and 4;

with the proviso that the sum of nₐ+n_{b} is not less than 2 and not greater than 4;

or X₁ and Z₁ together form a fused ring system of the formula (la):

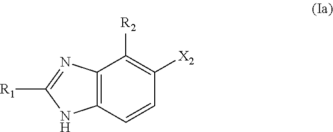

Rₐ is selected from the group of hydrogen and C₁-C₃ alkyl;

X₂ is selected from the group of:

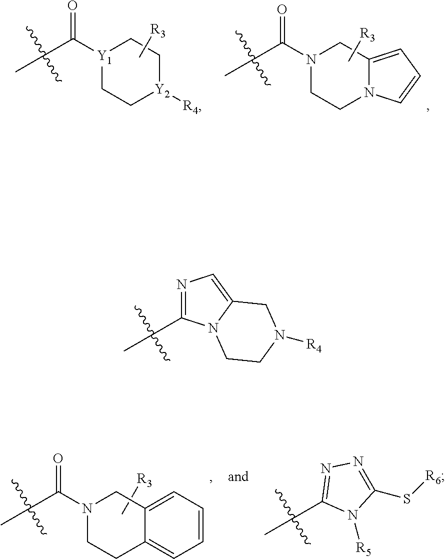

the wavy line [symbol] in each instance represents a bond through which each X₁ and X₂ moiety is bound;

Y₁ is selected from the group of C and N;

Y₂ is selected from the group of C, N, S, and O, provided R₄ is not present when Y₂ is O and provided R₄ is either not present or present one or two times when Y₂ is S;

with the proviso that no more than one of Y₁ and Y₂ are C;

Z₁, Z₂, and Z₃ are each independently selected from the group of C and N, with the proviso that no more than two of Z₁, Z₂, and Z₃ may be N, and with the further proviso that Z₁, Z₂, and Z₃, when bound to R₂, are C;

R₁ is selected from the group of C₁-C₆ alkyl, —(CH₂),ₙ₁-C₃-C₆ cycloalkyl, —NR^{x}R^{y}, phenyl, and benzyl, wherein the C₁-C₆ alkyl group and the rings of the —(CH₂),ₙ₁-C₃-C₆ cycloalkyl, phenyl, and benzyl groups are substituted by 0, 1, 2, or 3 substituents selected from the group of halogen, OH, CF₃, and —O—C₁-C₃ alkyl, and wherein each of R^{x} and R^{Y} are independently selected from the group of H and C₁-C₆ alkyl substituted by 0, 1, 2, or 3 substituents selected from the group of halogen, OH, CF₃, and —O—C₁-C₃ alkyl;

n1 is an integer selected from the group of 0, 1, 2, and 3;

R₂ is selected from the group of phenyl and a monocyclic heterocyclic ring or a bicyclic or spirocyclic heterocyclic ring system bound through a nitrogen heteroatom and containing 3, 4, 5, 6, 7, or 8 ring carbon atoms and 0, 1, 2, 3, or 4 additional ring heteroatom selected from the group of N and O, with the R₂ monocyclic ring or a bicyclic or spirocyclic ring system substituted by 0, 1, 2, or 3 substituents selected from the group of C₁-C₆ alkyl, —O—C₁-C₆ alkyl, —(CH₂)ₙ₁—C₃-C₆cycloalkyl, —CF₃, halogen, and phenyl;

R₃ is present one or more times and is independently selected from the group of:
a) hydrogen;
b) —CO₂H or —CO₂—(C₁-C₆ alkyl);
c) C₁-C₆ alkyl substituted by 0, 1, 2, 3, 4 or 5 substituents selected from the group of halogen, CF₃, and OH;
d) phenyl, benzyl, C₃-C₆ cycloalkyl, and —CH₂-C₃-C₆ cycloalkyl, the rings of each of the phenyl, benzyl, C₃-C₆ cycloalkyl, and —CH₂-C₃-C₆ cycloalkyl groups being substituted by 0, 1, 2, or 3 substituents selected from OH, halogen, and C₁-C₆ alkyl, wherein the C₁-C₆ alkyl group is further substituted by 0, 1, 2, 3, 4 or 5 substituents selected from the group of halogen, —CF₃, and OH;
e) a 5-membered or 6-membered heterocyclic ring containing 1, 2, or 3 ring heteroatoms independently selected from O, S, and N, the 5-membered or 6-membered heterocyclic ring being substituted by 0, 1, 2, or 3 substituents selected from OH, halogen, benzyl, and C₁-C₆ alkyl, wherein the C₁-C₆ alkyl group is further substituted by 0, 1, 2, 3, 4 or 5 substituents selected from the group of halogen and OH;

R₄ is present one or two times and is independently selected from the group of H, oxo, C₁-C₆ alkyl,—(CH₂),ₙ₂—C₃-C₆cycloalkyl, —O—(CH₂),ₙ₂—C₃-C₆cycloalkyl, —C(═O)—O—C₁-C₆ alkyl, -S(═O)₂—C₁-C₆ alkyl, —C(═O)—NR^{x}R^{y}, phenyl, benzyl, or a heterocyclic ring having 3, 4, 5, 6, 7, 8, 9, or 10 ring atoms of which 1, 2, 3, or 4 ring atoms are selected from the group of N, O, and S, wherein each of R^{x} and R^{Y} are independently selected from the group of H and C₁-C₆ alkyl substituted by 0, 1, 2, or 3 substituents selected from halogen, OH, CF₃, and —O—C₁-C₃ alkyl; and, when Y₂ is carbon, R₄ may also be —O—C₁-C₆ alkyl or two R₄ may form a carbocycle or heterocycle;

wherein the R₄ C₁-C₆ alkyl, —C(═O )—O—C₁-C₆ alkyl, and —O—C₁-C₆ alkyl groups and the rings of the —(CH₂),ₙ₂—C₃-C₆cycloalkyl and —O—(CH₂)—-C₃-C6cycloalkyl, phenyl, and benzyl groups are each independently substituted by 0, 1, 2, or 3 substituents selected from the group of halogen, CF₃, OH, a heterocyclic ring having 3, 4, 5, 6, 7, 8, 9, or 10 ring atoms of which 1, 2, 3, or 4 ring atoms are selected from the group of N, O, and S, substituted or unsubstituted phenyl, and —O—C₁-C₃ alkyl, with the proviso that when Y₁ is nitrogen and Y₂ is carbon, then R₄ is not unsubstituted benzyl and with the proviso that when Y₁ is nitrogen and Y₂ is nitrogen, then R₄ is not unsubstituted pyridinyl or substituted or unsubstituted phenyl;

R₅ is selected from the group of H and C₁-C₆ alkyl, wherein the R₅ C₁-C₆ alkyl group is further substituted by 0, 1, 2, 3, 4 or 5 substituents selected from the group of halogen, —CF₃, —NR^{x}R^{y}, and OH, wherein each of R^{x} and R^{Y} are independently selected from the group of H and C₁-C₆ alkyl substituted by 0, 1, 2, or 3 substituents selected from the group of halogen, OH, CF₃, and —O—C₁-C₃ alkyl;

R₆ is selected from the group of H, C₁-C₆ alkyl, a heterocyclic ring having 3, 4, 5, 6, 7, 8, 9, or 10 ring atoms of which 1, 2, 3, or 4 ring atoms are selected from the group of N, O, and S, phenyl, and benzyl, wherein the R₆ C₁-C₆ alkyl groups is further substituted by 0, 1, 2, 3, 4 or 5 substituents selected from the group of halogen, —CF₃, —NR^{x}R^{y}, and OH, and the rings of the R₆ phenyl and benzyl groups and the heterocyclic ring are substituted by 0, 1, 2, 3, 4 or 5 substituents selected from the group of C₁-C₆ alkyl, —O—C₁-C₆ alkyl, halogen, —CF₃, and OH, wherein each of R^{x} and R^{Y} are independently selected from the group of H and C₁-C₆ alkyl substituted by 0, 1, 2, or 3 substituents selected from halogen, OH, CF₃, and —O—C₁-C₃ alkyl; and

n₂ in each instance is an integer selected from the group of 0, 1, 2, and 3;

with the proviso that, when R₂ is an unsubstituted azepanyl ring ortho to X₂, X₁ is the moiety

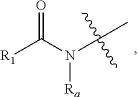

Rₐ is H, R₁ is unsubstituted cyclopropyl, X₂ is

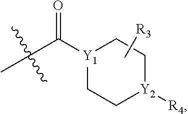

and Y₁ is N, and either a) Y₂ is O, ) Y2 is N and R4 is H or alkyl, c) Y₂ is C and R4 is H, alkyl, or —C(═O)13 O—C₁-C₆ alkyl, then R₃ is not H.

### BRIEF DESCRIPTION OF THE SEVERAL VIEWS OF THE DRAWINGS

FIG. 1 depicts blood pressure (BP) recordings from wild type (WT) (n=5; circles) and GPR39 KO (n=10; squares) mice on normal (0.4%), low (0.1%) and high-salt (4.0%) diet. BP was recorded continuously using implantable pressure-sensing telemetry. Data was collected every 5 minutes for 10 seconds for at least 96 hours for each experimental condition. BP is reported as 24-hour, daytime, and nighttime mean arterial pressure (MAP).

FIG. 2 depicts data demonstrating dose-dependent effects of GPR39 antagonist EXAMPLE 34 on BP in the spontaneously hypertensive rat (SHR). Decrease in BP was expressed as a percentage of baseline BP (*p<0.05).

FIG. 3A depicts the timeline of experimental design. EXAMPLE 34 was given 15 min after left coronary occlusion as a single dose during a 45 min occlusion followed by 2 hr of reperfusion.

FIG. 3B depicts coronary capillary beds from area at risk (AAR) and normally perfused regions in WT and GPR39 KO mice. The endothelium was immunolabeled for CD31 and pericytes for NG2. Within the AAR there were less constricted capillaries in the KO animal.

FIG. 3C depicts infarct size (TTC staining) from WT (n=6) and GPR39 KO (n=6) and shows a significant reduction in infarct size in the latter.

FIG. 3D depicts data from EXAMPLE 34 treated WT animals (n=6) and shows a significant reduction in infarct size compared to vehicle treated animals (n=5). Infarct size in both FIG. 3C and FIG. 3D is expressed as a percent of AAR defined by Evans Blue. Summary data are depicted to the right of actual images from single representative animals.

FIGS. 4A-4D depict no reflow data from WT, GPR39 KO, and EXAMPLE 34 mice. Thioflavin-S and Evans Blue were injected ante-mortem and measurement of no reflow (area without Evans Blue on the right panel). FIG. 4A depicts an example of a WT animal showing large regions of no reflow, while an example of a GPR39 KO mouse hardly shows no reflow.

FIG. 4B depicts summary data showing significant reduction in no reflow/AAR ration in GPR39 mice (n=4) compared with WT (N=4).

FIG. 4C depicts an example of a vehicle-treated WT animal showing large regions of no reflow, while an EXAMPLE 34-treated mouse hardly shows no reflow.

FIG. 4D depicts summary data showing significant reduction in no reflow/AAR ratio in EXAMPLE 34 treated mice (n=4) compared with vehicle treated mice (n=5). EXAMPLE 34 was given 15 min after coronary occlusion which lasted a total of 45 min followed by 2 hour reperfusion.

### DETAILED DESCRIPTION OF THE INVENTION

Within the scope of compounds of Formula (I), or a pharmaceutically acceptable salt thereof, there are two additional embodiments comprising, respectively, a compound of Formula (I-A), or a pharmaceutically acceptable salt thereof, and a compound of Formula (I-B), or a pharmaceutically acceptable salt thereof:

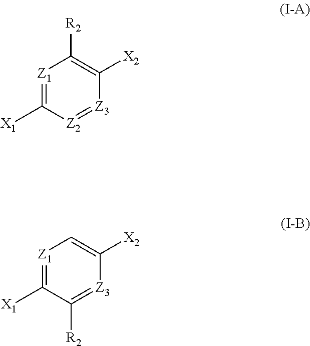

wherein, in each instance, when present, X₁, X₂, R₂, Z₁, Z₂, Z₃, and all other variables and provisos are as defined for Formula (I), above.

It is also understood that there are separate and independent embodiments within the scope of Formula (I) and based upon the definitions of X₁ and X₂, above, comprising, respectively, a compound of FIGS. (I-1), (I-2), (I-3), (I-4), and (I-5), or a pharmaceutically acceptable salt thereof:

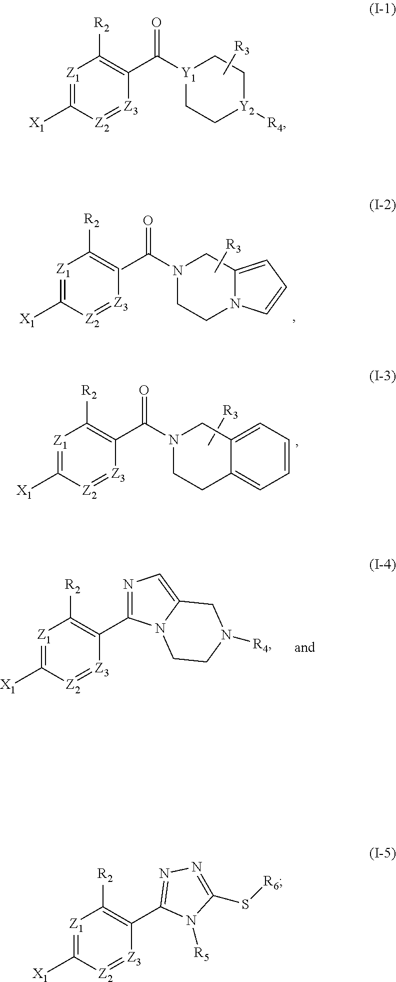

wherein, in each instance in which they are present, all variables, including X₁, Rₐ, R₁, R₂, R₃, R₄, Y₁, Y₂, Z₁, Z₂, Z₃, nₐ, n_{b}, n₁, n₂, and the provisos are as defined above for Formula (I).

It is also understood that there are separate and independent embodiments within the scope of Formula (I) and based upon the definitions of X₁ and X₂, above, comprising, respectively, a compound of Formulas (I-a) through (I-hh), below, or a pharmaceutically acceptable salt thereof:

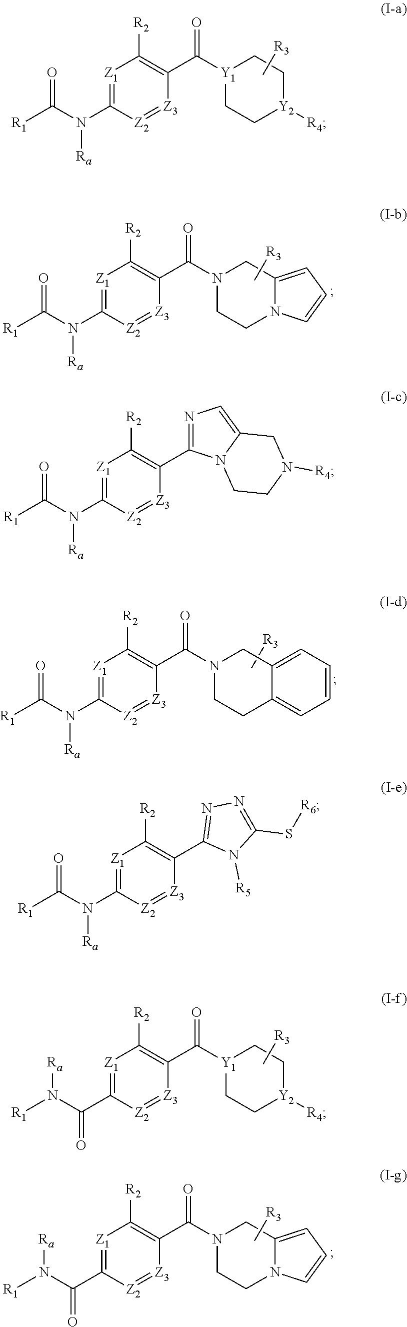

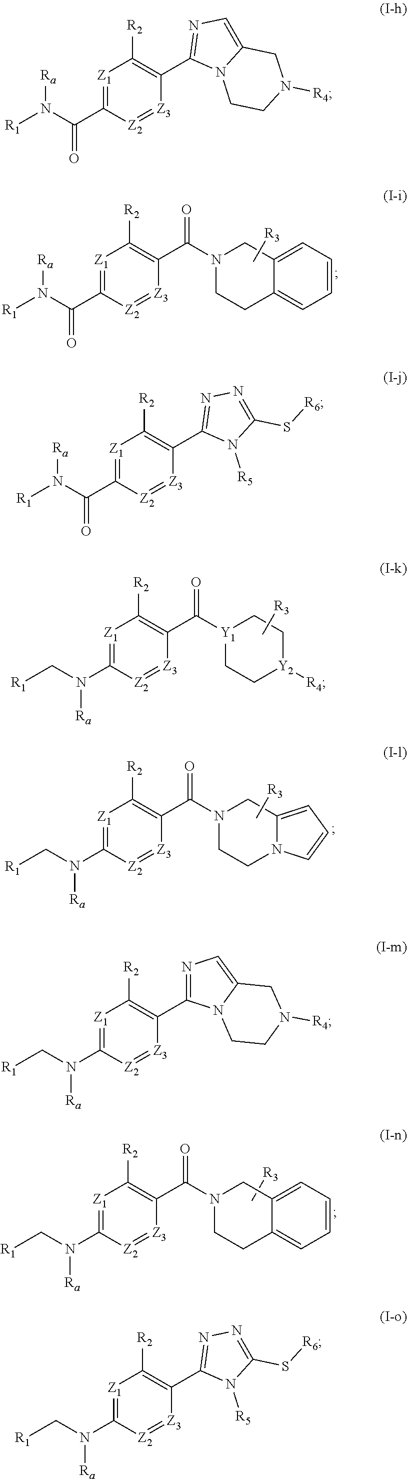

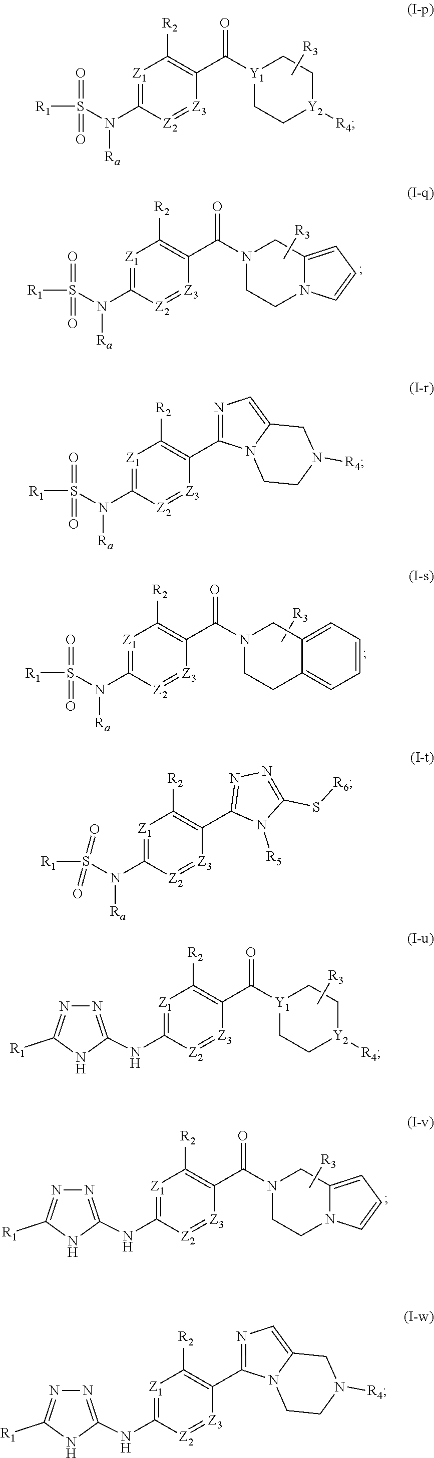

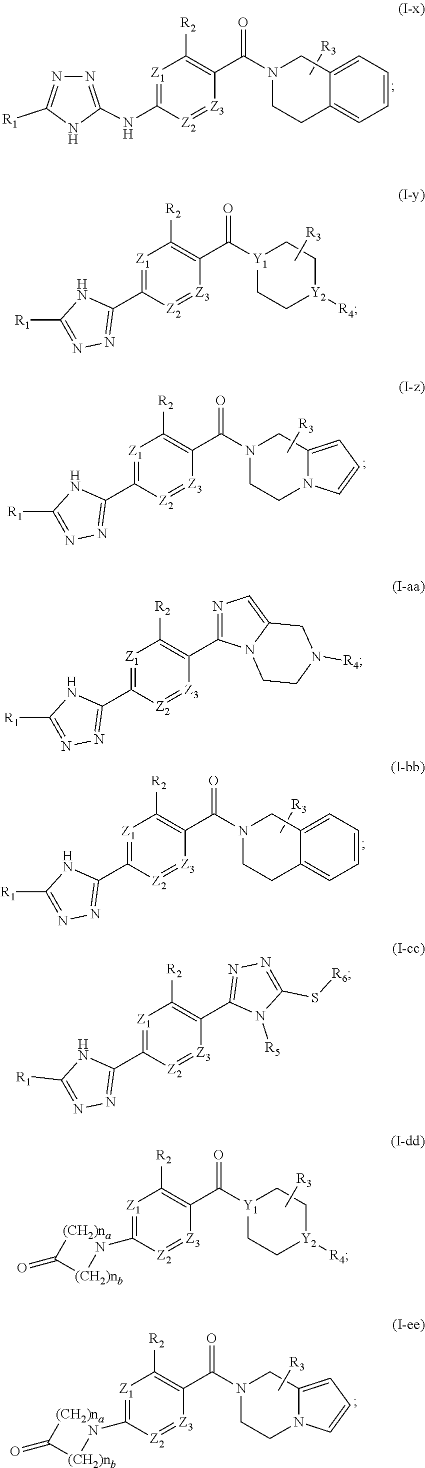

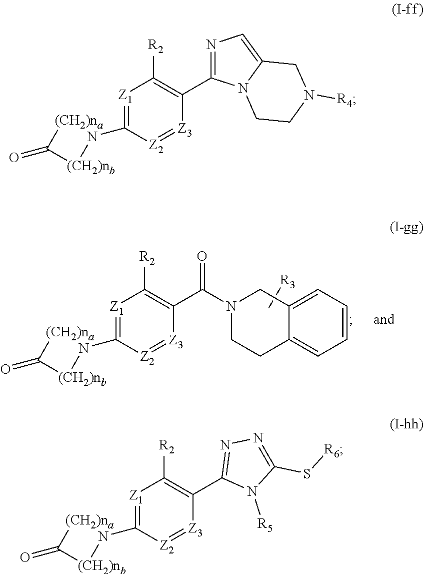

wherein, in each instance in which they are present, all variables, including Rₐ, R₁, R₂, R₃, R₄, R₅, R₆, Y₁, Y₂, Z₁, Z₂, Z₃, nₐ, n_{b}, n1, n2, and the provisos are as defined above for Formula (I).

Other separate embodiments provide a compound of each of Formula (I), Formulas (I-1) through (I-5), and Formulas (I-a) through (I-hh), above, or a pharmaceutically acceptable salt thereof, wherein in each embodiment Z₁, Z₂, and Z₃ are each C, and Rₐ, R₁, R₂, R₃, R₄, R₅, R₆, Y₁, Y₂, nₐ, n_{b}, n1, n2, and the provisos are as defined above for the corresponding Formula (I) through (I-hh).

Other separate embodiments provide a compound of each of Formula (I), Formulas (I-1) through (I-5), and Formulas (I-a) through (I-hh), above, or a pharmaceutically acceptable salt thereof, wherein in each embodiment Z₁, Z₂, and Z₃ are each N, and Rₐ, R₁, R₂, R₃, R₄, R₅, R₆, Y₁, Y₂, nₐ, n_{b}, n1, n2, and the provisos are as defined above for the corresponding Formula (I) through (I-hh).

Additional separate embodiments provide a compound of each of Formula (I), Formulas (I-1) through (I-5), and Formulas (I-a) through (I-hh), above, or a pharmaceutically acceptable salt thereof, wherein in each embodiment, Z₁ and Z₂ are each C, Z₃ is N, and Rₐ, R₁, R₂, R₃, R₄, R₅, R₆, Y₁, Y₂, nₐ, n_{b}, n1, n2, and the provisos are as defined above for the corresponding Formula (I) through (I-hh).

Additional separate embodiments provide a compound of each of Formula (I), Formulas (I-1) through (I-5), and Formulas (I-a) through (I-hh), above, or a pharmaceutically acceptable salt thereof, wherein in each embodiment, Z₁ and Z₃ are each C, Z₂ is N, and Rₐ, R₁, R₂, R₃, R₄, R₅, R₆, Y₁, Y₂, nₐ, n_{b}, n1, n2, and the provisos are as defined above for the corresponding Formula (I) through (I-hh).

Additional separate embodiments provide a compound of each of Formula (I), Formulas (I-1) through (I-5), and Formulas (I-a) through (I-hh), above, or a pharmaceutically acceptable salt thereof, wherein in each embodiment, Z₁ and Z₃ are each N, Z₂ is C, and Rₐ, R₁, R₂, R₃, R₄, R₅, R₆, Y₁, Y₂, nₐ, n_{b}, n1, n2, and the provisos are as defined above for the corresponding Formula (I) through (I-hh).

Additional separate embodiments provide a compound of each of Formula (I), Formulas (I-1) through (I-5), and Formulas (I-a) through (I-hh), above, or a pharmaceutically acceptable salt thereof, wherein in each embodiment, Z₁ and Z₂ are each N, Z₃ is C, and Rₐ, R₁, R₂, R₃, R₄, R₅, R₆, Y₁, Y₂, nₐ, n_{b}, n1, n2, and the provisos are as defined above for the corresponding Formula (I) through (I-hh).

Additional separate embodiments provide a compound of each of Formula (I), Formulas (I-1) through (I-5), and Formulas (I-a) through (I-hh), above, or a pharmaceutically acceptable salt thereof, wherein in each embodiment, Z₂ and Z₃ are each N, Z₁ is C, and Rₐ, R₁, R₂, R₃, R₄, R₅, R₆, Y₁, Y₂, nₐ, n_{b}, n1, n2, and the provisos are as defined above for the corresponding Formula (I) through (I-hh).

Within each of the embodiments herein, there is a further embodiment in which R₂ is selected from the group of:

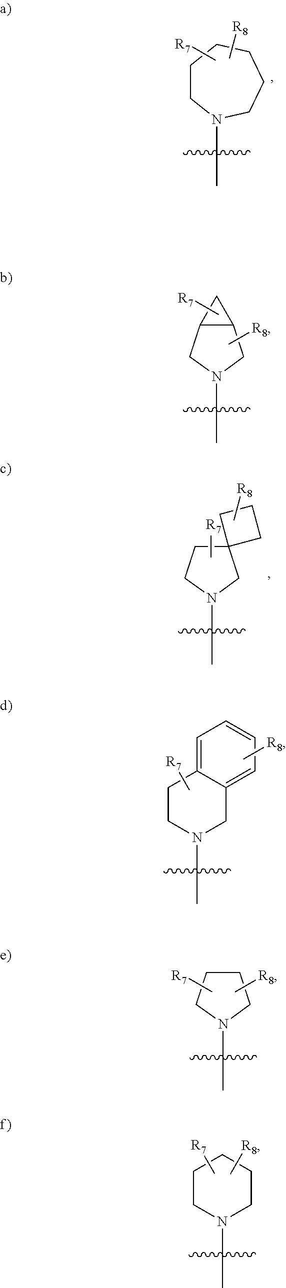

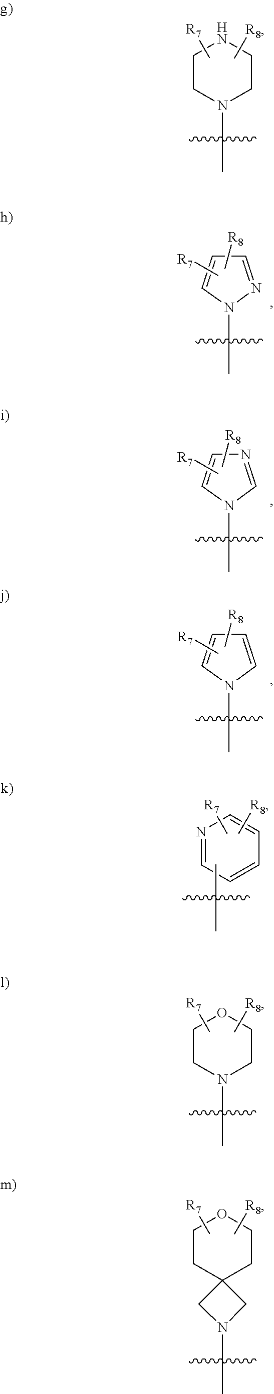

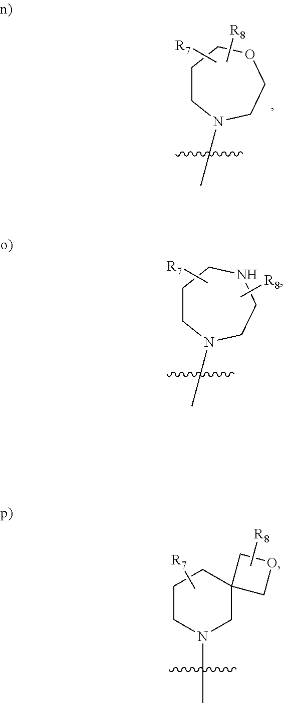

wherein R₇ and R₈ are in each instance independently selected from hydrogen, halogen, C₁-C₆ alkyl, —O—C₁-C₆ alkyl, —CF₃, and phenyl, with the proviso that only one of R₇ and R₈ may be phenyl. It is understood that in the embodiments herein, in instances wherein the R₂ group is a bicyclic or spirocyclic ring, R₇ and R₈ may be bound to any available ring carbon or nitrogen atom in either of the rings and that R₇ and R₈ may be bound to ring atoms in the same or different rings.

Within each of the embodiments herein, there is yet a further embodiment in which R₂ is selected from the group of:

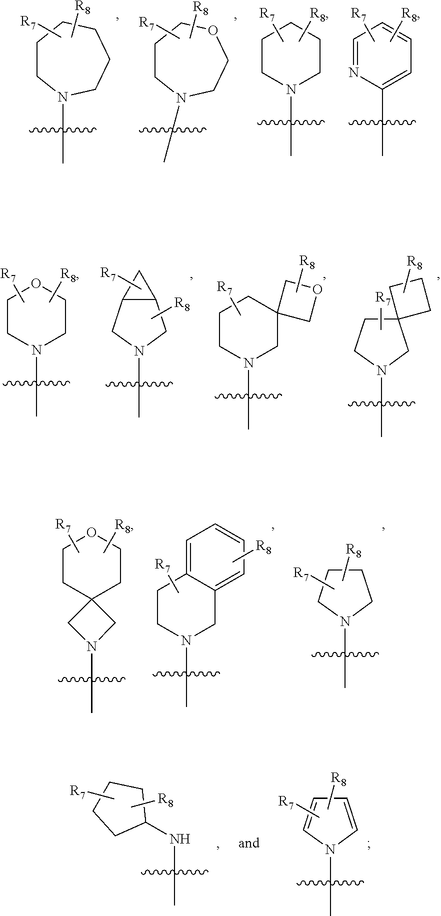

wherein R₇ and R₈ are in each instance independently selected from hydrogen, halogen, C₁-C₆ alkyl, —O—C₁-C₆ alkyl, —CF₃, and phenyl, with the proviso that only one of R₇ and R₈ may be phenyl.

Also provided are separate embodiments comprising, respectfully, compounds for each of the Formula (I), Formulas (I-1) through (I-5), and Formulas (I-a) through (I-hh), or a pharmaceutically acceptable salt thereof, wherein separate embodiments comprise a compound of each formula wherein R₂ is defined as each group a) through r), above. For instance, provided is a compound of Formula (I), or a pharmaceutically acceptable salt thereof, wherein R₂ is the azepane ring of group a) substituted by variable groups R₇ and R₈. Another embodiment comprises a compound of Formula (I), wherein R₂ is the azabicyclo[3.10.0]hexanyl ring of group b) substituted by variable groups R₇ and R₈. This pattern continues to define the remainder of the embodiments, each with the separate referenced R₂ group from the list above.

Within each of the embodiments above for a compound of each of Formula (I), Formulas (I-1) through (I-5), and Formulas (I-a) through (I-hh), or a pharmaceutically acceptable salt thereof, there is also a further embodiment wherein each variable, including Z₁, Z₂, Z₃, Rₐ, R₁, R₃, R₄, R₅, R₆, Y₁, Y₂, nₐ, n_{b}, n1, n2, and the provisos are as defined above for the corresponding Formula (I), Formulas (I-1) through (I-5), and Formulas (I-a) through (I-hh), and R₂ is selected from the group of:

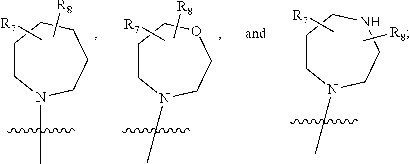

wherein R₇ and R₈ are each independently selected from hydrogen, halogen, C₁-C₆ alkyl, —O—C₁-C₆ alkyl, halogen, and —CF₃.

Within each of the embodiments above for a compound of each of Formula (I), Formulas (I-1) through (I-5), and Formulas (I-a) through (I-hh), or a pharmaceutically acceptable salt thereof, there is also a further embodiment wherein each variable, including Z₁, Z₂, Z₃, Rₐ, R₁, R₃, R₄, R₅, R₆, Y₁, Y₂,nₐ, n_{b}, n1, n2, and the provisos are as defined above for the corresponding Formula (I), Formulas (I-1) through (I-5), and Formulas (I-a) through (I-hh), and R₂ is selected from the group of:

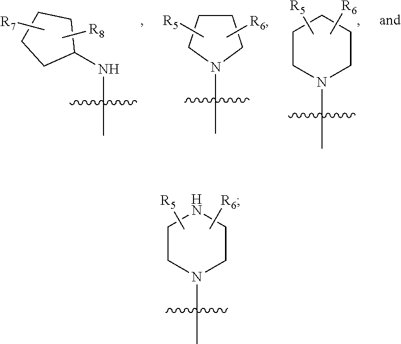

wherein R₇ and R₈ are each independently selected from hydrogen, halogen, C₁-C₆ alkyl, —O—C₁-C₆ alkyl, and —CF₃.

Within each of the embodiments above for a compound of each of Formula (I), Formulas (I-1) through (I-5), and Formulas (I-a) through (I-hh), or a pharmaceutically acceptable salt thereof, there is also a further embodiment wherein each variable, including Z₁, Z₂, Z₃, Rₐ, R₁, R₃, R₄, R₅, R₆, Y₁, Y₂, nₐ, n_{b}, n1, n2, and the provisos are as defined above for the corresponding Formula (I), Formulas (I-1) through (I-5), and Formulas (I-a) through (I-hh), and R₂ is selected from the group of:

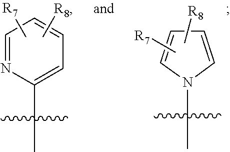

wherein R₇ and R₈ are each independently selected from hydrogen, halogen, C₁-C₆ alkyl, —O—C₁-C₆ alkyl, and —CF₃.

Within each of the embodiments above for a compound of each of Formula (I), Formulas (I-1) through (I-5), and Formulas (I-a) through (I-hh), or a pharmaceutically acceptable salt thereof, there is also a further embodiment wherein each variable, including Z₁, Z₂, Z₃, Rₐ, R₁, R₃, R₄, R₅, R₆, Y₁, Y₂, nₐ, n_{b}, n1, n2, and the provisos are as defined above for the corresponding Formula (I), Formulas (I-1) through (I-5), and Formulas (I-a) through (I-hh), and R₂ is selected from the group of:

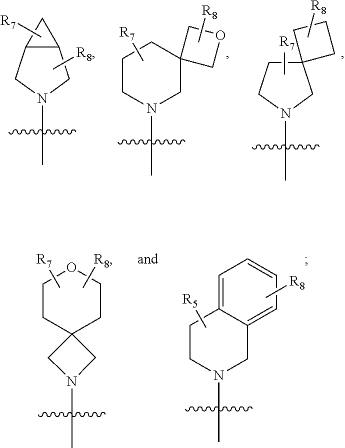

wherein R₇ and R₈ are each independently selected from hydrogen, halogen, C₁-C₆ alkyl, —O—C₁-C₆ alkyl, and —CF₃.

In some embodiments herein, R₇ and R₈ are selected from hydrogen, halogen, —CF₃, C₁-C₄ alkyl, and C₁-C₄ alkoxy. In other embodiments, R₇ and R₈ are selected from hydrogen, halogen, —CF₃, C₁-C₃ alkyl, and C₁-C₃ alkoxy. In still further embodiments, hydrogen, F, Cl, —CF₃, C₁-C₃ alkyl, and C₁-C₃ alkoxy.

Within each of the embodiments for compounds and salts herein comprising a R₃ moiety, there is a further embodiment of the same scope, except R₃ is pesent one to three times. Within each of the embodiments for compounds and salts herein comprising a R₃ moiety, there is a further embodiment of the same scope, except R₃ is present one to two times. Within each of the embodiments for compounds and salts herein comprising a R₃ moiety, there is a further embodiment of the same scope, except R₃ is present only one time. Within each of the embodiments for compounds and salts herein comprising a R₃ moiety, there is a further embodiment of the same scope, except R₃ is selected from the group of hydrogen, C₁-C₄ alkyl, C₃-C₆ cycloalkyl, —CF₃, —C₁-C₄ alkyl-OH, phenyl, pyrazolyl, and thiophenyl, wherein the phenyl, pyrazolyl, and thiophenyl rings are substituted by 0, 1, 2, or 3 substituents selected from the group of halogen, OH, —CF₃, C₁-C₄ alkyl, and C₁-C₄ alkoxy.

Within each of the embodiments for compounds and salts herein comprising a R₃ moiety, there is a further embodiment of the same scope, except R₃ is selected from the group of phenyl, pyrazolyl, and thiophenyl, substituted by 0, 1, 2, or 3 substituents selected from the group of halogen, OH, —CF₃, C₁-C₄ alkyl, and C₁-C₄ alkoxy.

Also included for each compound of Formula (I) and all other formulas and specifically named compounds herein are the pharmaceutically acceptable salts, pharmaceutically acceptable co-crystals, pharmaceutically acceptable esters, pharmaceutically acceptable solvates, hydrates, isomers (including optical isomers, racemates, or other mixtures thereof), tautomers, isotopes, polymorphs, and pharmaceutically acceptable prodrugs of such compounds.

The compounds of the disclosure may possess an asymmetric center, and can be produced as a racemic mixture or as individual enantiomers. The individual enantiomers may be obtained by asymmetric synthesis or by resolving a racemic or non-racemic mixture of an intermediate at some appropriate stage of the synthesis. The individual enantiomers may also be obtained by resolution of the compound by conventional means, such as crystallization in the presence of a resolving agent, or chromatography, using, for example a chiral high pressure liquid chromatography (HPLC) column. The individual enantiomers as well as racemic and non-racemic mixtures of enantiomers are within the scope of the present disclosure, all of which are intended to be included within the structures depicted in this specification unless otherwise specifically indicated.

### Methods of Use

Hypertension is the most common chronic disease, affecting 1.13 billion people and accounting for 10 million deaths every year worldwide. Despite the availability of antihypertensive drugs that successfully reduced blood pressure (BP) for millions of patients, important unmet needs remain. Only half of people with hypertension have their BP under control, and current antihypertensive medications fail to restore normal BP in a large proportion of patients. Treatment-resistant hypertension (TRH), also known as resistant hypertension, is on the rise due to the rising rates of diabetes and obesity, which contribute up to 75% of the risk for hypertension and to most cases of TRH. Identifying new targets and developing novel strategies for preventing and treating hypertension and its complications remain a high public health and medical priority.

Provided is a method of inhibiting the activity of a GPR39 protein in a subject, the method comprising administering to the subject in need thereof a pharmaceutically effective amount of a compound of Formula (I′), or a pharmaceutically acceptable salt thereof.

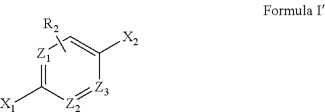

wherein:

X₁ is selected from the group of:

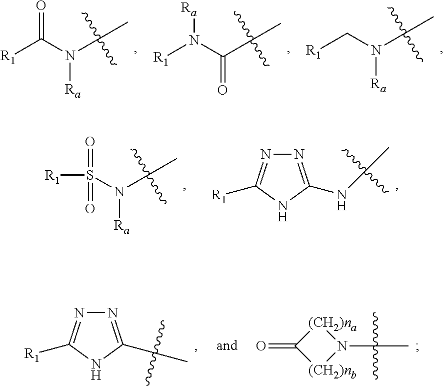

nₐ is an integer selected from the group of 0, 1, and 2;

n_{b}is an integer selected from the group of 0, 1, 2, 3, and 4;

with the proviso that the sum of nₐ +n_{b}is not less than 2 and not greater than 4;

or X₁ and Z₁ together form a fused ring system of the formula (la):

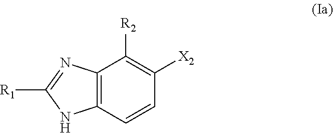

Rₐ is selected from the group of hydrogen and C₁-C₃ alkyl;

X₂ is selected from the group of:

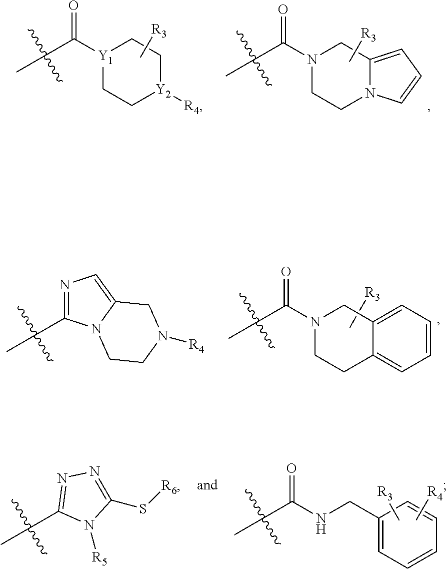

the wavy line [symbol] in each instance represents a bond through which each X₁ and X₂ moiety is bound;

Y₁ is selected from the group of C and N;

Y₂ is selected from the group of C, N, S, and O, provided R₄ is not present when Y₂ is O and provided R₄ is either not present or present one or two times when Y₂ is S;

with the proviso that no more than one of Y₁and Y₂ are C;

Z₁, Z₂, and Z₃ are each independently selected from the group of C and N, with the proviso that no more than two of Z₁, Z₂, and Z₃ may be N, and with the further proviso that Z₁, Z₂, and Z₃, when bound to R₂, are C;

R₁ is selected from one of the group of C₁-C₆ alkyl, —(CH₂)ₙ₁—C₃-C₆ cycloalkyl, —NR^{X}R^{Y}, phenyl, and benzyl, wherein the C₁-C₆ alkyl group and the rings of the —(CH₂)ₙ₁—C₃-C₆ cycloalkyl, phenyl, and benzyl groups are substituted by 0, 1, 2, or 3 substituents independently selected from the group of halogen, OH, CF₃, and —O—C₁-C₃ alkyl, and wherein each of R^{X} and R^{Y} are independently selected from the group of H and C₁-C₆ alkyl substituted by 0, 1, 2, or 3 substituents selected from the group of halogen, OH, CF₃, and —O—C₁-C₃ alkyl;

n1 is an integer selected from 0, 1, 2, and 3;

R₂ is selected from the group of phenyl and a monocyclic heterocyclic ring or a bicyclic or spirocyclic heterocyclic ring system bound through a nitrogen heteroatom and containing 3, 4, 5, 6, 7, or 8 ring carbon atoms and 0, 1, 2, 3, or 4 additional ring heteroatom selected from the group of N and O, with the R₂ monocyclic ring or a bicyclic or spirocyclic ring system substituted by 0, 1, 2, or 3 substituents selected from the group of C₁-C₆ alkyl, —O—C₁-C₆ alkyl, —(CH₂)ₙ₁—C₃-C₆cycloalkyl, —CF₃, halogen, and phenyl;

R₃ is present one or more times and is selected from the group of:
a) hydrogen;
b) —CO₂H or —CO₂—(C₁-C₆ alkyl);
c) C₁-C₆ alkyl substituted by 0, 1, 2, 3, 4 or 5 substituents selected from the group of halogen, CF₃, and OH;
d) phenyl, benzyl, C₃-C₆ cycloalkyl, and —CH₂-C₃-C₆ cycloalkyl, the rings of each of the phenyl, benzyl, C₃-C₆ cycloalkyl, and —CH₂-C₃-C₆ cycloalkyl groups being substituted by 0, 1, 2, or 3 substituents selected from OH, halogen, and C₁-C₆ alkyl, wherein the C₁-C₆ alkyl group is further substituted by 0, 1, 2, 3, 4 or 5 substituents selected from the group of halogen, —CF₃, and OH;
e) a 5-membered or 6-membered heterocyclic ring containing 1, 2, or 3 ring heteroatoms independently selected from O, S, and N, the 5-membered or 6-membered heterocyclic ring being substituted by 0, 1, 2, or 3 substituents selected from OH, halogen, benzyl, and C₁-C₆ alkyl, wherein the C₁-C₆ alkyl group is further substituted by 0, 1, 2, 3, 4 or 5 substituents selected from the group of halogen and OH;

R₄ is present one or two times and is independently selected from the group of H, oxo, C₁-C₆ alkyl,-(CH₂)ₙ₂-C₃-C₆cycloalkyl, —O—(CH₂)ₙ₂—C₃-C₆cycloalkyl, —C(═O)—O—C₁-C₆ alkyl, —S(═O)₂—C₁-C₆ alkyl, —C(═O)—NR^{X}R^{Y}, phenyl, benzyl, or a heterocyclic ring having 3, 4, 5, 6, 7, 8, 9, or 10 ring atoms of which 1, 2, 3, or 4 ring atoms are selected from the group of N, O, and S, wherein each of R^{X} and R^{Y} are independently selected from the group of H and C₁-C₆ alkyl substituted by 0, 1, 2, or 3 substituents selected from halogen, OH, CF₃, and —O—C₁-C₃ alkyl;

and, when Y₂ is carbon, R₄ may also be —O—C₁-C₆ alkyl or two R₄ may form a carbocycle or heterocycle;

wherein the R₄ C₁-C₆ alkyl, —(═O)—O—C₁-C₆ alkyl, and —O—C₁-C₆ alkyl groups and the rings of the —(CH₂)ₙ₂—C₃-C₆cycloalkyl and —O—(CH2)ₙ₂—C₃-C₆ cycloalkyl, phenyl, and benzyl groups are each independently substituted by 0, 1, 2, or 3 substituents selected from the group of halogen, CF₃, OH, a heterocyclic ring having 3, 4, 5, 6, 7, 8, 9, or 10 ring atoms of which 1, 2, 3, or 4 ring atoms are selected from the group of N, O, and S, substituted or unsubstituted phenyl, and —O—C₁-C₃ alkyl;

R₅ is selected from the group of H and C₁-C₆ alkyl, wherein the R₅ C₁-C₆ alkyl groups is further substituted by 0, 1, 2, 3, 4 or 5 substituents selected from the group of halogen, —CF₃, —NR^{x}R^{y}, and OH, wherein each of R^{X}and R^{Y} are independently selected from H and C₁-C₆ alkyl substituted by 0, 1, 2, or 3 substituents selected from halogen, OH, CF₃, and —O—C₁-C₃ alkyl;

R₆ is selected from the group of H, C₁-C₆ alkyl, a heterocyclic ring having 3, 4, 5, 6, 7, 8, 9, or 10 ring atoms of which 1, 2, 3, or 4 ring atoms are selected from the group of N, O, and S, phenyl, and benzyl, wherein the R₆ C₁-C₆ alkyl groups is further substituted by 0, 1, 2, 3, 4 or 5 substituents selected from the group of halogen, —CF₃, —NR^{x}R^{y}, and OH, and the rings of the R₆ phenyl and benzyl groups and the heterocyclic ring are substituted by 0, 1, 2, 3, 4 or 5 substituents selected from the group of C₁-C₆ alkyl, —O—C₁-C₆ alkyl, halogen, —CF₃, and OH, wherein each of R^{X} and R^{Y} are independently selected from H and C₁-C₆ alkyl substituted by 0, 1, 2, or 3 substituents selected from halogen, OH, CF₃, and —O—C₁-C₃ alkyl; and

n₂ in each instance is an integer selected from the group of 0, 1, 2, and 3.

That said, in certain embodiments, the method comprises administering to the subject in need thereof a pharmaceutically effective amount of a compound of Formula (I), of a compound of any subformula of Formula (I), of any of the specifically named compounds herein, or a pharmaceutically acceptable salt of any of the foregoing.

Provided is a method of treating hypertension in a human in need thereof, the method comprising administering to the human a pharmaceutically effective amount of a compound of Formula (I′), a compound of Formula (I), a compound of any subformula of Formula (I), any of the specifically named compounds herein, or a pharmaceutically acceptable salt of any of the foregoing. In one embodiment, the hypertension to be treated is primary hypertension, which may also be referred to as essential hypertension.

In another embodiment, the hypertension to be treated is secondary hypertension, including, but not limited to, hypertension caused by sleep apnea, blocked renal arteries, abnormal levels of hormones, enzymes, growth factors, or other agents controlling systemic or localized blood pressure (including, but not limited to, renin, angiotensin I and II, and aldosterone, angiotensin converting enzyme, catecholamines, thrombin, prostaglandins, natriuretic peptides, vasopressin, adreomedulin, Substance P, calcitonin gene related peptide, kallikreins, kininoogengs, kinins, kinin-degrading enzymes, phosducin, adipokines, and leptin), adrenal gland disease, thyroid abnormalities (including hyperthyroidism, hypothyroidism, Cushing's Disease, Pheochormocytoma, and excess growth hormone), and constrictions of the aorta, including coarctation.

In one embodiment, the hypertension to be treated is treatment-resistant or refractory hypertension.

In still another embodiment, the hypertension to be treated is hypertensive urgency.

In another embodiment, the hypertension to be treated is malignant hypertension, which may also be referred to as hypertensive emergency or hypertensive crisis.

In an additional embodiment, the hypertension to be treated is isolated systolic hypertension.

In a further embodiment, the hypertension to be treated is salt-sensitive hypertension. In another embodiment, the hypertension to be treated is pulmonary hypertension.

Current therapies for stroke involve dissolving or removing the clot responsible for vessel occlusion. Yet, despite opening large vessels, small blood vessels, remain compromised in a significant proportion of patients, ranging from 30-50%, which can lead to ineffective therapy. A compound as described herein can be combined with current stroke therapy to improve outcome for a large segment of patients.

Accordingly, provided is a method of treating stroke in a human in need thereof, the method comprising administering to the human a pharmaceutically effective amount of a compound of Formula (I′), a compound of Formula (I), a compound of any subformula of Formula (I), any of the specifically named compounds herein, or a pharmaceutically acceptable salt of any of the foregoing.

Vascular dementia a small vessel disease in the brain compromises blood flow and oxygen delivery to brain cells, resulting in neuronal dysfunction. Dementia due to small vessel disease is the second leading cause of dementia after Alzheimer's disease, and is the predominant cause in individuals over 70 and in developing countries. Provided is a method of treating of dementia due to small vessel disease in a human in need thereof, the method comprising administering to the human a pharmaceutically effective amount of a compound of Formula (I′), a compound of Formula (I), a compound of any subformula of Formula (I), any of the specifically named compounds herein, or a pharmaceutically acceptable salt of any of the foregoing.

In light of their effects on relaxing and protecting microvessels, the compounds and compositions herein may also be used in methods of treating conditions associated with microvascular dysfunction and microvascular disease, with each method comprising administering to a subject in need thereof a pharmaceutically effective amount of a compound of Formula (I′), or a compound of Formula (I), a compound of any subformula of Formula (I), any of the specifically named compounds herein, or a pharmaceutically acceptable salt of any of the foregoing. This includes methods of treating microvascular complications associated with or caused by diabetes. In one embodiment, the diabetes in question is Type I diabetes mellitus. In another embodiment the diabetes in question is Type II diabetes mellitus. This also includes methods of treating microvascular complications associated with or caused by prediabetic conditions, also known as prediabetes.

Also provided is a method of treating heart failure in a human, the method comprising administering to the human in need thereof a pharmaceutically effective amount of a compound of Formula (I′), a compound of Formula (I), a compound of any subformula of Formula (I), any of the specifically named compounds herein, or a pharmaceutically acceptable salt of any of the foregoing.

Included are methods of treating microvascular anginₐ, cerebral small vessel disease (cSVD), vascular cognitive impairment (VCI), Systemic Microvascular Endothelial Dysfunction (alone or presenting with Infective Endocarditis), and Chronic Kidney Disease.

Peripheral arterial disease, which is mostly small vessel disease and is generally not helped by the surgical and stenting techniques that target large vessels, but do not treat small vessel disease.

Provided herein is a method of treatment of peripheral artery disease in a human, the method comprising administering to a mammal in need thereof a pharmaceutically effective amount of a compound of Formula (I′), a compound of Formula (I), a compound of any subformula of Formula (I), any of the specifically named compounds herein, or a pharmaceutically acceptable salt of any of the foregoing.

The compounds herein may also be used in the treatment of preeclampsia, the pregnancy complication characterized by high blood pressure and signs of damage to another organ system, most often the liver and kidneys. Provided herein is a method of treatment of preeclampsia in a human, the method comprising administering to a mammal in need thereof a pharmaceutically effective amount of a compound of Formula (I′), a compound of Formula (I), a compound of any subformula of Formula (I), any of the specifically named compounds herein, or a pharmaceutically acceptable salt of any of the foregoing.

The compounds herein are also useful in the treatment of myocardial infarction, particularly in cases where no-reflow phenomenon is present. A no-reflow phenomenon is present when, despite restoration of the coronary flow of blood to the myocardium, breakdown of or obstruction to coronary microvasculature can markedly reduce blood flow to the infarct zone. Flow to the microcirculation in such instances may occur in one third of myocardial infarction patients.

As such, provided is a method of treatment of myocardial infarction in a human, the method comprising administering to a mammal in need thereof a pharmaceutically effective amount of a compound of Formula (I′), a compound of Formula (I), a compound of any subformula of Formula (I), any of the specifically named compounds herein, or a pharmaceutically acceptable salt of any of the foregoing.

Also provided is a method of treatment of myocardial infarction with the presence of no-reflow phenomenon in a human, the method comprising administering to a mammal in need thereof a pharmaceutically effective amount of a compound of Formula (I′), a compound of Formula (I), a compound of any subformula of Formula (I), any of the specifically named compounds herein, or a pharmaceutically acceptable salt of any of the foregoing.

The compounds of the present invention may also be used in situations of chronic coronary artery disease in which the large arteries are bypassed or stented and, yet, about a third of the patients continue to have angina due to microvascular dysfunction.

As such, provided is a method of treatment of chronic coronary artery disease in a human, the method comprising administering to a mammal in need thereof a pharmaceutically effective amount of a compound of Formula (I′), a compound of Formula (I), a compound of any subformula of Formula (I), any of the specifically named compounds herein, or a pharmaceutically acceptable salt of any of the foregoing.

Also provided is a method of treatment of microvascular dysfunction associated with chronic coronary artery disease in a human, the method comprising administering to a mammal in need thereof a pharmaceutically effective amount of a compound of Formula (I′), a compound of Formula (I), a compound of any subformula of Formula (I), any of the specifically named compounds herein, or a pharmaceutically acceptable salt of any of the foregoing.

The compounds of the present invention may also be used for conditions referred to as Syndrome X, wherein a subject is experiencing a cardiac microvascular dysfunction or constriction causing angina despite normal epicardial coronary artery activity, such as diagnosed by angiography. As such, provided is a method of treatment of Syndrome X in a human, the method comprising administering to a mammal in need thereof a pharmaceutically effective amount of a compound of Formula (I′), a compound of Formula (I), a compound of any subformula of Formula (I), any of the specifically named compounds herein, or a pharmaceutically acceptable salt of any of the foregoing.

Provided is a method of treating breast cancer in a human in need thereof, the method comprising administering to the human a pharmaceutically effective amount of a compound of Formula (I′), a compound of Formula (I), a compound of any subformula of Formula (I), any of the specifically named compounds herein, or a pharmaceutically acceptable salt of any of the foregoing. In some embodiments, the breast cancer treated in this method is ER negative breast cancer.

Also provided is a method of treating gastric adenocarcinomas in a human, the method comprising administering to the human in need thereof a pharmaceutically effective amount of a compound of Formula (I′), a compound of Formula (I), a compound of any subformula of Formula (I), any of the specifically named compounds herein, or a pharmaceutically acceptable salt of any of the foregoing.

Also provided is a method of promoting or enhancing colon epithelial function and tight junction barrier integrity in a human, the method comprising administering to the human in need thereof a pharmaceutically effective amount of a compound of Formula (I′), a compound of Formula (I), a compound of any subformula of Formula (I), any of the specifically named compounds herein, or a pharmaceutically acceptable salt of any of the foregoing.

Similarly provided is a method of treating ulcerative colon diseases in a human, the method comprising administering to the human in need thereof a pharmaceutically effective amount of a compound of Formula (I′), a compound of Formula (I), a compound of any subformula of Formula (I), any of the specifically named compounds herein, or a pharmaceutically acceptable salt of any of the foregoing. In one embodiment, the ulcerative colon disease to be treated is ulcerative colitis. In another embodiment, the ulcerative colon disease to be treated is Crohn Disease or Crohn's Disease. In still another embodiment, the ulcerative colon disease to be treated is Irritable Bowel Syndrome (IBS), which may also be referred to as colitis, enteritis, ileitis, or proctitis.

Also provided is a method of treating Inflammatory Bowel Disease (IBD) in a human, the method comprising administering to the human in need thereof a pharmaceutically effective amount of a compound of Formula (I′), a compound of Formula (I), a compound of any subformula of Formula (I), any of the specifically named compounds herein, or a pharmaceutically acceptable salt of any of the foregoing.

Also provided is a method of treating diarrhea in a human, the method comprising administering to the human in need thereof a pharmaceutically effective amount of a compound of Formula (I′), a compound of Formula (I), a compound of any subformula of Formula (I), any of the specifically named compounds herein, or a pharmaceutically acceptable salt of any of the foregoing.

As microvascular complications can inhibit uniform delivery of anesthetics, in some embodiments the compounds herein may be used in methods of distributing local, regional, or systemic delivery of anesthetics and improve their effects. As such, also provided is a method of enhancing the delivery of anesthetics to a human experiencing microvascular complications, the method comprising administering to the human in need thereof a pharmaceutically effective amount of a compound of Formula (I′), a compound of Formula (I), a compound of any subformula of Formula (I), any of the specifically named compounds herein, or a pharmaceutically acceptable salt of any of the foregoing, and a pharmaceutically effective amount of an anesthetic. In some embodiments, the compound of Formula (I′), a compound of Formula (I), a compound of any subformula of Formula (I), any of the specifically named compounds herein, or a pharmaceutically acceptable salt of any of the foregoing, is administered to the human prior to the administration of anesthetic.

### Pharmaceutical Compositions

Also provided herein is a pharmaceutical composition comprising a pharmaceutically effective amount of a compound of Formula (I′), a compound of Formula (I), a compound of any subformula of Formula (I), any of the specifically named compounds herein, or a pharmaceutically acceptable salt or co-crystal of any of the foregoing, and a pharmaceutically acceptable carrier or excipient. Additional pharmaceutical compositions comprise, respectively, a pharmaceutically effective amount of a compound selected from those of FIGS. (I-1), (I-2), (I-3), (I-4), (I-5), and each of the formulas from (I-a)-(I-hh), as well as the specifically named compounds herein, and a pharmaceutically acceptable carrier or excipient.

Further provided herein is the use of a compound of Formula (I′), a compound of Formula (I), a compound of any subformula of Formula (I), any of the specifically named compounds herein, or a pharmaceutically acceptable salt or co-crystal of any of the foregoing, in the preparation of a medicament. Additional uses include the compounds that comprise, respectively, a pharmaceutically effective amount of a compound selected from those of FIGS. (I-1), (I-2), (I-3), (I-4), (I-5), and each of the formulas from (I-a)-(I-hh), as well as the specifically named compounds herein, for use in the preparation of a medicament.

The term “carrier” refers to an excipient or vehicle that includes without limitation diluents, disintegrants, precipitation inhibitors, surfactants, glidants, binders, lubricants, and the like with which the compound is administered. Pharmaceutically acceptable carriers are generally described herein and also in “Remington's Pharmaceutical Sciences” by E. W. Martin. Examples of carriers include, but are not limited to, aluminum monostearate, aluminum stearate, carboxymethylcellulose, carboxymethylcellulose sodium, crospovidone, glyceryl isostearate, glyceryl monostearate, hydroxyethyl cellulose, hydroxyethyl cellulose, hydroxymethyl cellulose, hydroxyoctacosanyl hydroxystearate, hydroxypropyl cellulose, hydroxypropyl cellulose, hydroxypropyl methylcellulose, lactose, lactose monohydrate, magnesium stearate, mannitol, microcrystalline cellulose, poloxamer 124, poloxamer 181, poloxamer 182, poloxamer 188, poloxamer 237, poloxamer 407, povidone, silicon dioxide, colloidal silicon dioxide, silicone, silicone adhesive 4102, and silicone emulsion. It should be understood, however, that the carriers selected for the pharmaceutical compositions, and the amounts of such carriers in the composition, may vary depending on the method of formulation (e.g., dry granulation formulation, solid dispersion formulation).

Some examples of suitable excipients in an oral formulation include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl and propylhydroxy-benzoates; sweetening agents; and flavoring agents.

As used herein, a “pharmaceutically acceptable excipient” is a pharmaceutically acceptable vehicle that includes, without limitation, any and all carriers, solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

The pharmaceutical compositions may be administered in either single or multiple doses by any of the accepted modes of administration of agents having similar utilities, for example as described in those patents and patent applications incorporated by reference, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

One mode for administration is parenteral, particularly by injection. The forms in which the compound of Formula I, or a pharmaceutically acceptable salt or co-crystal thereof, may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline may also conventionally be used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating a compound according to the present disclosure in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. In some embodiments, for parenteral administration, sterile injectable solutions are prepared containing a therapeutically effective amount, e.g., 0.1 to 1000 mg, of the compound of Formula I, or a pharmaceutically acceptable salt or co-crystal thereof. It will be understood, however, that the amount of the compound actually administered usually will be determined by a physician, in the light of the relevant circumstances, including the condition to be treated, the chosen route of administration, the actual compound administered and its relative activity, the age, weight, and response of the individual subject, the severity of the subject's symptoms, and the like.

Oral administration is another route for administration of the compound of Formula I, or a pharmaceutically acceptable salt or co-crystal thereof. Administration may be via capsule or enteric coated tablets, or the like. In making the pharmaceutical compositions that include the compound of Formula I, or a pharmaceutically acceptable salt or co-crystal thereof, the active ingredient is usually diluted by an excipient and/or enclosed within such a carrier that can be in the form of a capsule, sachet, paper or other container. When the excipient serves as a diluent, it can be in the form of a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

In some embodiments, for parenteral administration, each dosage unit contains from 0.1 mg to 1 g, 0.1 mg to 700 mg, or 0.1 mg to 100 mg of a compound of Formula I, or a pharmaceutically acceptable salt or co-crystal thereof. In some embodiments, a therapeutically effective amount or a pharmaceutically effective amount of a compound of Formula (I), or a pharmaceutically acceptable salt thereof, comprises from about 0.1 mg to about 500 mg per dose, given once or twice daily. In some embodiments, the individual dose is selected from 1 mg, 5 mg, 10 mg, 20 mg, 30 mg, 40 mg, 50 mg, 60 mg, 75 mg, 100 mg, 150 mg, 200 mg, 250 mg, 300 mg, 350 mg, 400 mg, and 500 mg per dose.

For any of the dosage units as described herein, it will be understood, however, that the amount of the compound actually administered usually will be determined by a physician, in the light of the relevant circumstances, including the condition to be treated, the chosen route of administration, the actual compound administered and its relative activity, the age, weight, and response of the individual subject, the severity of the subject's symptoms, and the like.

### Articles of Manufacture and Kits

Compositions (including, for example, formulations and unit dosages) comprising the compound of Formula I′ or I, or a pharmaceutically acceptable salt or co-crystal thereof, can be prepared and placed in an appropriate container, and labeled for treatment of an indicated condition. Accordingly, provided is also an article of manufacture, such as a container comprising a unit dosage form of the compound of Formula I′ or I, or a pharmaceutically acceptable salt or co-crystal thereof, and a label containing instructions for use of the compounds. In some embodiments, the article of manufacture is a container comprising a unit dosage form of the compound of Formula or I, or a pharmaceutically acceptable salt or co-crystal thereof, and at least one pharmaceutically acceptable vehicle. The article of manufacture may be a bottle, vial, ampoule, single-use disposable applicator, or the like, containing the pharmaceutical composition provided in the present disclosure. The container may be formed from a variety of materials, such as glass or plastic and in one aspect also contains a label on, or associated with, the container which indicates directions for use in the treatment of cancer or inflammatory conditions. It should be understood that the active ingredient may be packaged in any material capable of improving chemical and physical stability, such as an aluminum foil bag. In some embodiments, diseases or conditions indicated on the label can include, for example, treatment of cancer.

### Definitions

Terms such as “microvascular tone”, “microcirculatory tone”, “microcirculatory vascular tone”, and “arteriolar tone” refer to the degree of constriction experienced by a microcirculatory blood vessel relative to its maximally dilated state. Microcirculation may be anatomically defined as blood vessels having a diameter of between about 250 μm to about 100 μm, particularly including those between about 200 μm to about 150 μm, and includes arterioles, capillaries, and venules (post-capillary venules). Collectively, these vessels may be referred to as “microvessels”, “microcirculatory vessels”, and the like. In embodiments herein, it is a goal to provide compounds, compositions, and methods for relaxing microvessels in a subject, reducing vascular resistance and protecting the microvessels, themselves, and reducing or elimination end-organ damage associated with microvascular resistance, particularly that seen in the eyes, kidneys, and heart, as well as damage to nerve sheaths associated with microvessels. In some embodiments, the microcirculation is defined as blood vessels <200 μm in diameter. In the human body approximately 90% of the volume of blood resides in these vessels that include arterioles, capillary and venules. Arterioles range in diameter of approximately from 50-200 μm. Some venules contain vascular smooth muscle, and some capillaries are surrounded by pericytes, both of which are contractile cells that allow these vessels to constrict and relax, thus allowing more or less blood and oxygen to be delivered to cells, and cell waste removed. The size of these arterioles is tightly regulated to meet the oxygen demand of the cells they supply.

“Coronary microvascular disease” is heart disease that affects the walls and inner lining of tiny blood vessels branching from larger coronary arteries. Coronary microvascular disease may also be referred to as “Cardiac Syndrome X” or “nonobstructive coronary heart disease”. In the heart or elsewhere it may also be referred to as “small artery disease”, “small vessel disease”, or “arteriolosclerosis.”

Such diseases of the small vessels are often seen in primary hypertension, stroke, and hypertensive renal disease. Manifestations as kidney lesions typically involve afferent arterioles and the interlobular artery and may involve intimal thickening, vascular smooth muscle cell proliferation, and extracellular matrix deposition, increasing the media-to-lumen ration. Areas of vascular smooth muscle cells may then be replaced by fibrosis and cell loss.

“Microvascular angina” refers to a form of chest pain due to abnormalities in cardiac microvessels, including, but not limited to, faulty relaxation of or spasms therein.

A “heterocycle” or “heterocyclic group” herein refers to a chemical ring containing carbon atoms and at least one ring heteroatom selected from O, S, and N, including saturated, unsaturated, partially saturated, and aromatic rings.

Examples of 3-membered heterocycles, such as seen in the definitions of R₃ and R₄ herein, include by way of example and not limitation aziridinyl, azirinyl, oxiranyl, and thiiranyl groups.

Examples of 4-membered heterocycles, such as seen in the definitions of R₃ and R₄ herein, include by way of example and not limitation azetidinyl, dihydro-1A⁴-azetyl, azetyl, 1,3-diazetidinyl, and oxetanyl groups.

Examples of 5-membered heterocycles, such as seen in the definitions of R₃ and R₄ herein, include by way of example and not limitation thiazolyl, tetrahydrothiophenyl, sulfur oxidized tetrahydrothiophenyl, furanyl, thienyl, pyrrolyl, dihydropyrrolyl, pyrazolyl, imidazolyl, tetrazolyl, pyrrolidinyl, 2-pyrrolidonyl, dihydropyrrolidinyl, pyrrolinyl, tetrahydrofuranyl, triazinyl, thienyl, 2H-pyrrolyl, isothiazolyl, isoxazolyl, imidazolidinyl, imidazolinyl, pyrazolidinyl, pyrazolinyl, and oxazolidinyl groups.

Examples of 6-membered heterocycles, such as seen in the definitions of R₃ and R₄ herein, include by way of example and not limitation pyridyl, dihydroypyridyl, tetrahydropyridyl (piperidyl), pyrimidinyl, piperidinyl, thiadiazinyl, thiazinyl, 2H,6H-1,5,2-dithiazinyl, pyranyl, pyrazinyl, pyridazinyl, piperazinyl, thienyl, thiopyran, dithanyl, morpholinyl, thiomorpholinyl, groups

Examples of 7-membered heterocycles herein, include by way of example and not limitation borepinyl, azepanyl, azepinyl, oxepanyl, oxepinyl, theipinyl, thiepanyl, diazepanyl, diazepinyl, and thiazepinyl groups.

Examples of 8-membered heterocycles herein, include by way of example and not limitation azocinyl, azocinyl, oxocanyl, oxocinyl, thiocanyl, and thiocinyl groups.

Examples of 9-membered heterocycles herein, include by way of example and not limitation monocyclic heterocycles such as azonanyl, azoninyl, oxonanyl, oxoninyl, thionanyl, and thioninyl groups and fused heterocycles, such as indolyl, indolinyl, isoindolyl, indolizinyl, indazolyl, azaindolyl, benzimidazolyl, azaindazolyl, pyrazolopyrimidinyl, purinyl, benzofuranyl, isobenzofuranyl, benzothiophenyl, benzo[d]isoxazolyl, benzo[d]isothiazolyl, benzo[d]oxazolyl, benzo[c][1,2,5]thiadiazolyl, benzo[d]thiazolyl, benzisothiazolyl, adeninyl, and guaninyl groups.

Examples of 10-membered heterocycles herein, include by way of example and not limitation decahydroisoquinolinyl, decahydroquinolinyl, tetrahydroquinolinyl, quinolinyl, dihydroquinolinyl, isoquinolinyl, quinoxalinyl, quinolizinyl, phthalazinyl, quinazolinyl, cinnolinyl, chromenyl, isochromenyl, naphthyridinyl, pyrido[3,2-d]pyrimidinyl, pyrido[4,3-d]pyrimidinyl, pyrido[3,4-b]pyrazinyl, pyrido[2,3-b]pyrazinyl, pteridinyl, benzo[e][1,2]oxazinyl, benzo[e][1,3]oxazinyl, benzo[b][1,4]oxazinyl, quinolinonyl, thienoppyrimidinyl (including thieno[3,2-d]pyrimidinyl), and isoquinolinonyl groups.

It is understood that the general terms for the heterocycles referenced herein include each isomeric form of the heterocycle, such as the term “dithianyl” including 1,2 dithianyl, 1,3-dithianyl, and 1,4-dithianyl groups, the term “thiadiazinyl” including 1,2,5 thiadiazineyl and 1,3,4-thiadiainyl groups, the term “azaindolyl” including 4-azaindolyl, 5-azaindolyl, 6-azaindolyl, and 7-azaindolyl groups, and “benzothiophenyl” including benzo[b]thiophenyl and benzo[c]thiophenyl groups.

Similarly, general heterocycle names include each variance in one or more points of unsaturation. For instance, the term “dihydropyrrolyl” refers to “2,3-dihydro-1H-pyrrolyl” and “2,5-dihydro-1H-pyrrolyl” groups.

The term “alkyl” refers to a straight or branched hydrocarbon. For example, an alkyl group can have a specified number of chain carbons, such as Ito 6 carbon atoms (i.e., C₁-C₆ alkyl or C₁₋₆alkyl). Examples of suitable alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (-C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH2CH₂CH₂CH₃), 2-hexyl (—CH(CH₃)CH₂CH₂CH₂CH₃), 3-hexyl (—CH(CH₂CH₃)(CH₂CH₂CH₃)), 2- methyl-2-pentyl (—C(CH₃)₂CH₂CH₂CH₃), 3-methyl-2-pentyl (—CH(CH₃)CH(CH₃)CH₂CH₃), 4-methyl-2-pentyl (-CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), and 3,3-dimethyl-2-butyl (—CH(CH₃)C(CH₃)₃.

The term “alkoxy” refers to a group having the formula “—O—alkyl,” in which an alkyl group, as defined above, is attached to the parent molecule via an oxygen atom. The alkyl portion of an alkoxy group can have a specified number of carbon chain atoms, such as 1 to 6 carbon atoms (i.e., C₁-C₆ alkoxy or C1-6 alkoxy). Examples of suitable alkoxy groups include, but are not limited to, methoxy (-O—CH₃ or —OMe), ethoxy (—OCH₂CH₃ or —OEt), t-butoxy (—O—C(CH₃)₃ or —OtBu) and the like.

The term “cycloalkyl” refers to a saturated ring having 3 to 6 carbon atoms as a monocycle, including cyclopropyl, cyclobutyl, cyclopentyl, and cyclohexyl groups.

The term “halogen” refers to an atom selected from the group of elements chlorine, fluorine, bromine, and iodine.

The term “oxo” refers to double-bonded oxygen “═O”.

The terms “therapeutically effective amount” and “pharmaceutically effective amount” refer to an amount that is sufficient to effect treatment, as defined below, when administered to a subject (e.g., a mammal, such as a human) in need of such treatment. The therapeutically or pharmaceutically effective amount will vary depending upon the subject and disease condition being treated, the weight and age of the subject, the severity of the disease condition, the manner of administration and the like, which can readily be determined by one of ordinary skill in the art. For example, a “therapeutically effective amount” or a “pharmaceutically effective amount” of a compound of Formula I, or a pharmaceutically acceptable salt or co-crystal thereof, is an amount sufficient to antagonize GPR39 expression or activity, and thereby treat a subject (e.g., a human) suffering an indication, or to ameliorate or alleviate the existing symptoms of the indication. For example, a therapeutically or pharmaceutically effective amount may be an amount sufficient to decrease a symptom of a disease or condition responsive to inhibition of GPR39 activity.

“Treatment” or “treating” is an approach for obtaining beneficial or desired results including clinical results. Beneficial or desired clinical results may include one or more of the following: (i) inhibiting the disease or condition (e.g., decreasing one or more symptoms resulting from the disease or condition, and/or diminishing the extent of the disease or condition); (ii) slowing or arresting the develoμment of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or (iii) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival).

The terms “inhibiting” or “inhibition” indicates a decrease, such as a significant decrease, in the baseline activity of a biological activity or process. “Inhibition of GPR39 activity” refers to a decrease in GPR39 activity as a direct or indirect response to the presence of a compound of Formula I, or a pharmaceutically acceptable salt or co-crystal thereof, relative to the activity of GPR39 in the absence of such compound or a pharmaceutically acceptable salt or co-crystal thereof. The decrease in activity may be due to the direct interaction of the compound with GPR39, or due to the interaction of the compound(s) described herein with one or more other factors that in turn affect GPR39 expression and/or activity. For example, the presence of the compound(s) may decrease GPR39 activity by directly binding to the GPR39, by causing (directly or indirectly) another factor to decrease GPR39 expression or activity, or by (directly or indirectly) decreasing the amount of GPR39 protein present in the cell or organism. In some embodiments, the inhibition of GPR39 activity may be compared in the same subject prior to treatment, or other subjects not receiving the treatment. The term “inhibitor” is understood to refer to a compound or agent that, upon administration to a human in need thereof at a pharmaceutically or therapeutically effective dose, provides the inhibition activity desired.

“Delaying” the develoμment of a disease or condition means to defer, hinder, slow, retard, stabilize, and/or postpone develoμment of the disease or condition. This delay can be of varying lengths of time, depending on the history of the disease or condition, and/or subject being treated. A method that “delays” develoμment of a disease or condition is a method that reduces probability of disease or condition develoμment in a given time frame and/or reduces the extent of the disease or condition in a given time frame, when compared to not using the method. Such comparisons are typically based on clinical studies, using a statistically significant number of subjects. Disease or condition develoμment can be detectable using standard methods, such as routine physical exams, mammography, imaging, or biopsy. Develoμment may also refer to disease or condition progression that may be initially undetectable and includes occurrence, recurrence, and onset.

As used herein, the singular terms “a,” “an,” and “the” include plural referents unless context clearly indicates otherwise. Similarly, the word “or” is intended to include “and” unless the context clearly indicates otherwise. Also, as used herein, the term “comprises” means “includes.” Hence “comprising A or B” means including A, B, or A and B.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed and/or claimed herein are inclusive of the recited endpoint and independently combinable (for example, the ranges of “from 2 to 10” and “2-10” are inclusive of the endpoints, 2 and 10, and all the intermediate values 3, 4, 5, 6, 7, 8, and 9).

By “significant” is meant any detectable change that is statistically significant in a standard parametric test of statistical significance such as Student's T-test, where p<0.05.

“Pharmaceutically acceptable salts” include, for example, salts with inorganic acids and salts with an organic acid. Examples of salts may include hydrochloride, phosphate, diphosphate, hydrobromide, sulfate, sulfinate, nitrate, malate, maleate, fumarate, tartrate, succinate, citrate, acetate, lactate, methanesulfonate (mesylate), benzenesuflonate (besylate), p-toluenesulfonate (tosylate), 2-hydroxyethylsulfonate, benzoate, salicylate, stearate, and alkanoate (such as acetate, HOOC—(CH₂)ₙ—COOH where n is 0-4). In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts.

The term “crystal forms” and related terms herein refer to the various crystalline modifications of a given substance, including, but not limited to, polymorphs, solvates, hydrates, co-crystals, and other molecular complexes, as well as salts, solvates of salts, hydrates of salts, other molecular complexes of salts, and polymorphs thereof. Crystal forms of a substance can be obtained by a number of methods, as known in the art. Such methods include, but are not limited to, melt recrystallization, melt cooling, solvent recrystallization, recrystallization in confined spaces such as, e.g., in nanopores or capillaries, recrystallization on surfaces or templates, such as, e.g., on polymers, recrystallization in the presence of additives, such as, e.g., co-crystal counter-molecules, desolvation, dehydration, rapid evaporation, rapid cooling, slow cooling, vapor diffusion, sublimation, grinding and solvent-drop grinding.

The term “co-crystal” or “co-crystal salt” as used herein means a crystalline material composed of two or more unique solids at room temperature, each of which has distinctive physical characteristics such as structure, melting point, and heats of fusion, hygroscopicity, solubility, and stability. A co-crystal or a co-crystal salt can be produced according to a per se known co-crystallization method. The terms co-crystal (or cocrystal) or co-crystal salt also refer to a multicomponent system in which there exists a host API (active pharmaceutical ingredient) molecule or molecules, such as a compound of Formula I, and a guest (or co-former) molecule or molecules. In particular embodiments the pharmaceutically acceptable co-crystal of the compound of Formula I or of the compound of Formula II with a co-former molecule is in a crystalline form selected from a malonic acid co-crystal, a succinic acid co-crystal, a decanoic acid co-crystal, a salicylic acid co-crystal, a vanillic acid co-crystal, a maltol co-crystal, or a glycolic acid co-crystal. Co-crystals may have improved properties as compared to the parent form (i.e., the free molecule, zwitter ion, etc.) or a salt of the parent compound. Improved properties can include increased solubility, increased dissolution, increased bioavailability, increased dose response, decreased hygroscopicity, a crystalline form of a normally amorphous compound, a crystalline form of a difficult to salt or unsaltable compound, decreased form diversity, more desired morphology, and the like.

As used herein, the terms “isotope” and “isotopic” in reference to a compound as disclosed herein means that one or more atoms of the compound is replaced with an isotope of such one or more atoms. An “isotope” refers to any of two or more forms of a chemical element, having the same number of protons in the nucleus, but having different numbers of neutrons in the nucleus. For example, an isotopic compound includes a compound in which one or more hydrogen atoms (H) has been replaced with one or more deuterium atoms (D). In this example, deuterium is an isotope of hydrogen, and replacing a hydrogen atom with deuterium (at one or more positions) renders the resulting compound an isotopic compound. For example, and in reference to Formula (I), replacing the two methyl groups of the isopropyl moiety (—CH(CH₃)₂) with fully deuterated methyl groups (—CH(CD₃)₂) would be an isotopic compound of Formula (I). In addition to replacing hydrogen with deuterium, other stable (non-radioactive) isotope substitutions include replacing carbon 12 with carbon 13, while unstable (radioactive) isotopes include replacing hydrogen with tritium, replacing carbon 12 with carbon 14, replacing iodine 127 with iodine 123 or iodine 125, and the like. Accordingly, all reference herein to isotopic compounds of Formula (I), as well as all reference to the various embodiments thereof, refers to a compound having one or more isotopic substitutions, including (but not limited to) substitutions of one or more hydrogen atoms with one or more deuterium atoms and any occurrence(s) in the compound. To this end, the isotopic compounds disclosed herein provide improved advantages relative to their non-isotopic forms. To this end, isotopic modification provides a means of improving existing drugs and/or as a tool in the design of new drugs. For example, isotopic drug design has proven successful in the context of the deuterium (D) kinetic isotope effect. Due to the twofold higher mass of D compared with H, the C-D bond is much more resistant toward oxidative processes (such as its ability to be catalyzed by CYP450 or by other enzymes involved in metabolism), while retaining very similar steric properties. Therefore, H-D isosteric replacement usually retains the pharmacodynamics of the compound, while improving its pharmacokinetics with a repercussion on half-life and/or of area under the curve values and, ultimately, on dose and/or dosing regimen. For example, drug exposure may be enhanced with isotopic modification, and/or a decrease of clearance. Such benefits are provided to the compounds disclosed hereby by way their isotopic derivation.

Terms such as “subject” and “patient” refer to an animal, such as a mammal, that has been or will be the object of treatment, observation or experiment. The methods described herein may be useful in both human therapy and veterinary applications. In some embodiments, the subject is a mammal; in some embodiments the subject is human; and in some embodiments the subject is chosen from cats and dogs. “Subject in need thereof” or “human in need thereof” refers to a subject, such as a human, who may have or is suspected to have diseases or conditions that would benefit from certain treatment; for example treatment with a compound of Formula I, or a pharmaceutically acceptable salt or co-crystal thereof, as described herein. This includes a subject who may be determined to be at risk of or susceptible to such diseases or conditions, such that treatment would prevent the disease or condition from developing.

The terms “prediabetes” or “prediabetic condition” refers to a condition in which a subject's blood sugar levels are not high enough to be considered diabetic but may be a precursor to Type 2 diabetes. A prediabetic condition may be defined in subjects having a fasting blood glucose level of 100 mg/dl or more, but less than 126 mg/dl (the level which is diagnostic for diabetes). Hemoglobin Mc (FlbAlc) levels are another laboratory test for diabetes. FlbAlc levels of 5.5% or greater are characteristic of diabetes, while levels from 5.7% to 6.4% suggest prediabetes.

### EXAMPLES

### Measurement of Intracellular Calcium Response in PC3 Cells, Expressing the Endogenous Human GPR39 Receptor

Human prostate adenocarcinoma (PC3) cells endogenously expressing human GPR39 receptor were seeded into black walls clear-base 384-well plates at a density of 7,500 cells per well in DM EM (Low Glucose) supplemented with 10% heat-inactivated foetal bovine serum and 1% of Pen/Strep and grown overnight at 37° C., 5% CO2. After washing with assay buffer (20 mM HEPES, 137 mM NaCI, 5.4 mM KCI, 10 mM glucose, 0.8 mM MgSO4, 1.3 mM CaCl2, 0.3 mM Na2HPO4, 0.4 mM KH2PO4 4.2 mM NaHCO3, pH 7.4), cells were incubated with Calcium-6 probe (Molecular Devices) at 37° C. for 2h and then equilibrated at RT for 30 minutes on metal block. The fluorescence was measured in real time by Fluorometric Imaging Plate Reader (FLIPR TETRA, Molecular Devices) (excitation wavelength at 470-495 nm, emission wavelength at 515-575 nm).

Compounds of invention were serially diluted in neat DMSO at 1000 fold concentrations and plated into 384-well plates and then 200-fold diluted in assay buffer containing 150 uM ZnCl2 (30 μM final concentration) and 1.1% DMSO (0.32% final concentration).

A dual addition FLIPR protocol was used which included a first addition of compounds 200-fold diluted in Assay Buffer containing 150 uM ZnCl2 and 1.1% DMSO and then after 10 minutes a second addition of a submaximal concentration of the hGPR39 receptor agonist, C3 (Tocris, TC-G 1008) at EC90 (concentration producing 90% of the maximal response) value of 1.5 μM.

Fluorescence was monitored throughout the run assay. The peak of Ca2+stimulation (baseline subtracted) was expressed as percentage of the maximal inhibition of the EC90 C3 response and was plotted versus the concentration of test compound. Curve fitting and IC50 estimations are carried out using a four-parameter logistic model using XLfit Software.

The potency of compounds was reported as functional pKi (—Log10Ki) obtained using the modified Cheng-Prusoff relationship (Cheng, Y., Prusoff, W. H. 1973.)

fKi =IC50/(1+[L]/EC50) where IC50 is the concentration of antagonist required for 50% inhibition of the maximum response, [L] is the concentration of the agonist used (EC90) and EC50 is the concentration of agonist need to induced 50% of the maximum response (obtained in each experimental plate).

fpKi data is shown in Table 1 below. An fpKi less than 5.2 is reported as “+/±”. An fpKi ranging from 5.2 to 5.9 is reported as “+”. An fpKi from 6.0 to 6.5 is reported as “++”. An fpKi from 6.6 to 7.2 is reported as “+++”. An fpKi equal to or greater than 7.3 is reported as “++++”.

TABLE 1

| Example No. | fpKi activity |
|---|---|
| 1 | ++++ |
| 2 | ++++ |
| 3 | ++ |
| 4 | ++ |
| 5 | ++ |
| 6 | + |
| 7 | + |
| 8 | +++ |
| 9 | + |
| 10 | + |
| 11 | ++ |
| 12 | ++ |
| 13a | +++ |
| 13b | +++ |
| 14a | +++ |
| 14b | +++ |
| 15 | +++ |
| 16 | +++ |
| 17 | +++ |
| 18 | ++ |
| 19 | ++ |
| 20 | ++ |
| 21 | ++ |
| 22 | +++ |
| 23 | ++ |
| 24 | +++ |
| 25 | ++ |
| 26 | +++ |
| 27 | ++ |
| 28 | +++ |
| 29 | +++ |
| 30 | ++ |
| 31 | ++ |
| 32 | ++ |
| 33 | + |
| 34 | + |
| 35 | +++ |
| 36 | +++ |
| 37 | ++ |
| 38 | +++ |
| 39 | +++ |
| 40 | ++ |
| 41 | + |
| 42 | ++ |
| 43 | ++ |
| 44 | + |
| 45 | ++ |
| 46 | ++ |
| 47 | +++ |
| 48 | ++ |
| 49 | +++ |
| 50 | +++ |
| 51 | +++ |
| 52 | +++ |
| 53 | ++ |
| 54 | +++ |
| 55 | ++ |
| 56 | + |
| 57 | + |
| 58 | +++ |
| 59 | +++ |
| 60 | +++ |
| 61 | +++ |
| 62a | ++++ |
| 62b | + |
| 62c | +++ |
| 62d | +++ |
| 63a | ++++ |
| 63b | ++++ |
| 63c | ++++ |
| 64a | ++++ |
| 64b | + |
| 64c | ++++ |
| 64d | ++ |
| 65 | ++++ |
| 66a | ++++ |
| 66b | ++ |
| 66c | ++++ |
| 67 | ++++ |
| 68a | ++++ |
| 68b | ++ |
| 68c | ++++ |
| 68d | +++ |
| 68e | +++ |
| 68f | ++++ |
| 69a | + |
| 69b | ++++ |
| 69c | ++++ |
| 70a | + |
| 70b | ++++ |
| 70c | +++ |
| 72a | + |
| 72b | ++++ |
| 73a | ++++ |
| 73b | + |
| 73c | ++++ |
| 74 | + |
| 75 | ++ |
| 76 | +++ |
| 77 | + |
| 79 | + |
| 81 | ++ |
| 82 | ++++ |
| 83 | +++ |
| 84a | +++ |
| 84b | + |
| 84c | +++ |
| 85 | ++++ |
| 86a | + |
| 86b | ++++ |
| 86c | ++++ |
| 87 | ++ |
| 88a | +++ |
| 88b | + |
| 88c | +++ |
| 89 | ++ |
| 90 | + |
| 91 | + |
| 92 | ++ |
| 93 | ++ |
| 94 | ++ |
| 95 | + |
| 96 | + |
| 98 | + |
| 102 | +++ |
| 103 | ++ |
| 104a | ++ |
| 104b | +++ |
| 105 | +++ |
| 106 | + |
| 107 | ++++ |
| 108 | +++ |
| 109 | + |
| 110 | +++ |
| 111 | + |
| 112 | ++ |
| 113 | + |
| 114a | +++ |
| 114b | +++ |
| 114c | +++ |
| 115 | ++ |
| 116 | + |
| 117 | + |
| 118 | ++ |
| 119 | +/− |
| 121 | + |
| 123 | + |
| 142 | ++++ |
| 143 | ++++ |
| 144 | +++ |
| 145 | +++ |
| 146 | +++ |
| 147 | +++ |
| 148a | +++ |
| 148b | + |
| 148c | +++ |
| 149a | +++ |
| 149b | + |
| 149c | +++ |
| 150a | +++ |
| 150b | + |
| 150c | +++ |
| 151a | +++ |
| 151b | + |
| 151c | +++ |
| 152 | ++ |
| 153 | +++ |
| 154 | +++ |
| 155a | ++ |
| 155b | ++ |
| 155c | ++ |
| 156 | +++ |
| 157 | + |
| 158 | ++++ |
| 166 | + |
| 167 | +++ |
| 168 | ++ |
| 169 | + |
| 170a | ++++ |
| 170b | ++ |
| 170c | ++++ |
| 171b | ++++ |
| 171c | ++++ |
| 172 | +++ |
| 173 | ++ |
| 174 | +++ |
| 175 | ++ |
| 176 | ++ |
| 177a | +++ |
| 177b | ++ |
| 177c | ++++ |
| 177d | ++ |
| 178 | +++ |
| 179 | ++++ |
| 180 | ++++ |
| 181 | ++++ |
| 182 | ++ |
| 183a | ++++ |
| 183b | +++ |
| 183c | +++ |
| 184 | +++ |
| 185a | +++ |
| 185b | ++++ |
| 185c | +++ |
| 186 | ++ |
| 187 | ++ |
| 188 | + |
| 189 | +++ |
| 191 | + |
| 195 | +++ |
| 196 | + |
| 199a | ++++ |
| 199b | ++ |
| 199c | +++ |
| 199d | ++ |
| 199e | ++++ |
| 200 | + |
| 201a | +++ |
| 201c | +++ |
| 202a | + |
| 202b | ++++ |
| 202c | +++ |
| 203 | ++ |
| 204 | ++ |
| 205 | +++ |
| 206 | +++ |
| 207 | ++ |
| 208 | ++++ |
| 209 | ++++ |
| 210 | ++++ |
| 211 | +++ |
| 212 | +++ |
| 213a | ++++ |
| 213b | +++ |
| 213c | +++ |
| 214 | ++++ |
| 215 | +++ |
| 216 | +++ |
| 217 | ++ |
| 218 | + |
| 219 | +++ |
| 220 | +++ |

### Synthesis

The compounds described herein may be prepared by methods known in the art and are exemplified by the following non-limiting descriptions.

Variations of moieties at the R₂ position may be prepared using Synthetic Route 1 below.

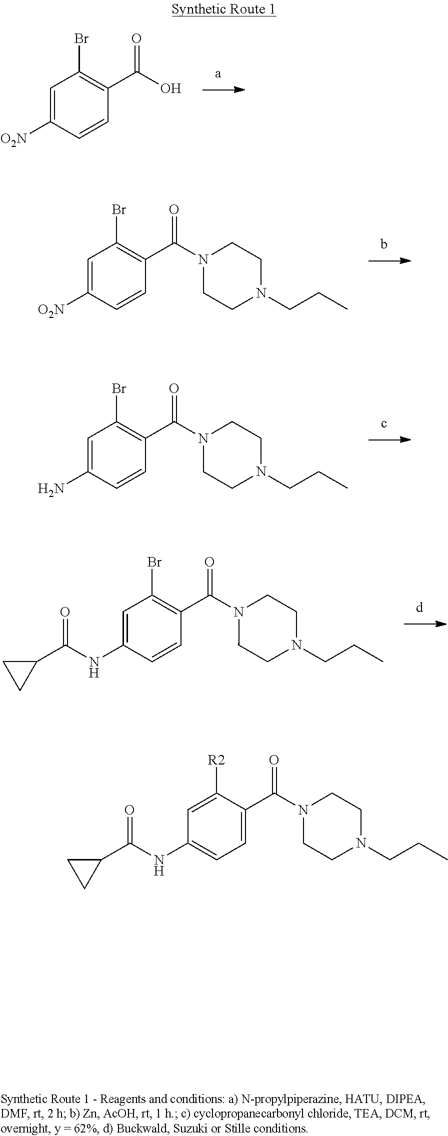

Preparation: 1-(2-bromo-4-nitrobenzoyl)-4-propylpiperazine

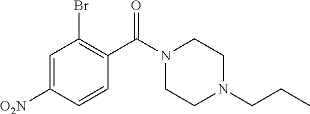 [SMILES: CCCN1CCN(C(=O)c2ccc([N+](=O)[O-])cc2Br)CC1]

To a solution of 2-bromo-4-nitrobenzoic acid (5.0 g, 20.32 mmol) and HATU:[dimethylamino(3-triazolo [4,5-b]pyridinyloxy)methylidene]-dimethylammonium;hexafluorophosphate (8.5 g, 22.36 mmol) in DMF (50 mL) N,N-Diisopropylethylamine (5.31 mL, 30.49 mmol) was added and the mixture was stirred at RT for 30 min. After this time a solution of 1-propylpiperazinedihydrobromide (5.9 g, 20.32 mmol) and N,N-Diisopropylethylamine (7.08 mL, 40.65 mmol) in DMF (25 mL) was added and the resulting solution was stirred for 2h at RT. The mixture was diluted with a s.s. of NaHCO₃ and extracted with AcOEt 3 times. The organic layer was washed with Brine, dried, filtered and concentrated under vacuum. The crude was purified by FC on silica gel (eluting from 100% of DCM to DCM/MeOH 9:1) affording the product of formula (1-(2-bromo-4-nitrobenzoyl)-4-propylpiperazine).
Yield: 7 g

¹ NMR (400 MHz, Chloroform-d) δ 8.48 (d, 1H), 8.24 (dd, 1H), 7.46 (d, 1H), 3.91 -3.83 (m, 2H), 2.63-2.45 (m, 4H), 2.40-2.31 (m, 4H), 1.57 -1.46 (m, 2H), 0.93 (t, 3H); LC-MS: m/z 356.11, 358.14 (MH+). ps Preparation: 3-bromo-4-(4-propylpiperazine-1-carbonyl)aniline

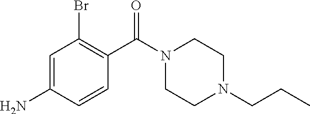 [SMILES: CCCN1CCN(C(=O)c2ccc(N)cc2Br)CC1]

(2-bromo-4-nitrophenyl)-(4-propylpiperazin-1-yl)methanone (6.63 g, 18.61 mmol) was dissolved in Acetic acid (51.13 mL) and zinc (6.08 g, 93.06 mmol) was added to the solution. The resulting suspension was stirred for 60 min at RT. After this time the suspension was filtered and the solution concentrated under vacuum. The residue was taken up with a s.s. of NaHCO₃ and extracted with AcOEt 3 times. The organic layers were collected, washed with Brine, dried over Na₂SO₄, filtered and concentrated under vacuum. The resulting material was purified by FC on NH column (from DCM to DCM/AcOEt 7:) affording the product of formula (4-amino-2-bromophenyl)-(4-propylpiperazin-1-yl)methanone.
Yield: 3.2 g

¹ NMR (400 MHz, DMSO-d₆) δ 6.91 (d, 1H), 6.78 (d, 1H), 6.56 (dd, 1H), 5.58 (s, 2H), 3.57 (s, 2H), 3.15 (s, 2H), 2.44-2.21 (m, 6H), 1.49-1.39 (m, 2H), 0.85 (t, 3H); LC-MS: m/z 326.44, 328.44 (MH+) ps Preparation: N-[3-bromo-4-(4-propylpiperazine-1-carbonyl)phenyl]cyclopropanecarboxamide

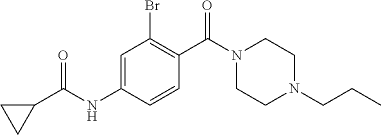 [SMILES: CCCN1CCN(C(=O)c2ccc(NC(=O)C3CC3)cc2Br)CC1]

To a solution of (4-amino-2-bromophenyl)-(4-propylpiperazin-1-yl)methanone (2.2 g, 6.74 mmol) in DCM (30 mL) triethylamine (2.82 mL, 20.23 mmol) was added and the resulting mixture was cooled to 0° C. Then a solution of cyclopropanecarbonyl chloride (1.53 mL, 16.86 mmol) in DCM (5 mL) was added dropwise and the reaction was stirred ON at RT. The day after the reaction mixture was diluted with DCM and washed with a s.s. of NaHCO₃ and brine. The organic phase was dried over phase separator and the solvent removed under vacuum. The resulting material was purified by FC on silica gel (eluting from DCM to DCM/MeOH 95:5) affording the product of formula N-[3-bromo-4-(4-propylpiperazine-1-carbonyl)phenyl]cyclopropanecarboxamide.
Yield: 2.15 g ¹ NMR (400 MHz, Chloroform-d) δ 8.28 (bs, 1H), 7.73 (d, 1H), 7.35 (dd, 1H), 7.10 (d, 1H), 3.88-3.79 (m, 2H), 3.37-3.16 (m, 2H), 2.61-2.26 (m, 6H), 1.62-1.46 (m, 3H), 1.13-1.04 (m, 2H), 0.99-0.82 (m, 5H); LC-MS: m/z 394.09, 396.11 (MH+) ps Preparation: N-(4-(4-propylpiperazine-1-carbonyl)-3-(6-azaspiro[3.4]octan-6-yl)phenypcyclopropanecarboxamide

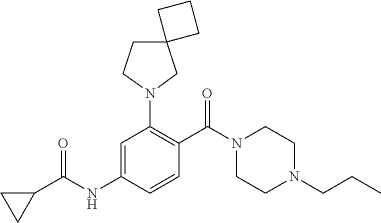 [SMILES: CCCN1CCN(C(=O)c2ccc(NC(=O)C3CC3)cc2N2CCC3(CCC3)C2)CC1]

### Example 1

In a vial were sequentially added N-[3-bromo-4-(4-propylpiperazine-1-carbonyl)phenyl]cyclopropanecarboxamide (100.0 mg, 0.250 mmol), sodium tert-butoxide (48.74 mg, 0.510 mmol) and dry Toluene (1.5 mL). The suspension was degassed with Schlenk line technique and 6-aza-spiro[3.4]octane (42.29 mg, 0.380 mmol) was added to the suspension. Then (1E,4E)-1,5-diphenyl-3-penta-1,4-dienone;palladium (11.61 mg, 0.010 mmol) and [1-(2-diphenylphosphino-1-naphthalenyI)-2-naphthalenyl]-diphenylphosphine (15.79 mg, 0.030 mmol) were added to the suspension and degassed again with Schlenk line technique for 10 min. The resulting mixture was heated to 120° C. ON. The day after the reaction mixture was diluted with water and extracted with AcOEt 3 times. The organic layers were collected and washed with Brine, dried over Na₂SO₄, filtered and the solvent removed under vacuum. The resulting material was purified by FC on RP using basic conditions (eluting from 100% of ammonium bicarbonate aqueous solution adjusted to pH 10 with ammonia to 100% of CH₃CN) affording the product of formula N-(4-(4-propylpiperazine-1-carbonyl)-3-(6-azaspiro[3.4]octan-6-yl)phenyl)cyclopropanecarboxamide.
Yield: 11 mg

¹ NMR (500 MHz, METHANOL-d4) δ ppm 7.14 (d, 1 H), 7.01 (d, 1 H), 6.87 (dd, 1 H), 3.98-3.86 (m, 1 H), 3.69-3.57 (m, 1 H), 3.38-3.30 (m, 2 H), 3.29-3.11 (m, 4 H), 2.66-2.58 (m, 1 H), 2.53-2.42 (m, 2 H), 2.40-2.26 (m, 3 H), 2.11-1.86 (m, 8 H), 1.79-1.71 (m, 1 H), 1.61-1.50 (m, 2 H), 0.98-0.81 (m, 7 H); LC-MS: m/z 425.3 (MH)⁺

Preparation: (S)-N-(3-(3-isopropylpyrrolidin-1-yl)-4-(4-propylpiperazine-1-carbonyl)phenyl)cyclopropanecarboxamide

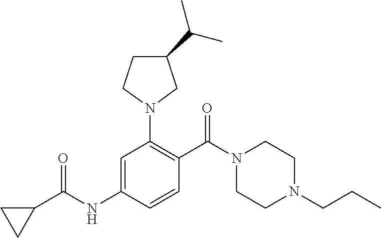 [SMILES: CCCN1CCN(C(=O)c2ccc(NC(=O)C3CC3)cc2N2CC[C@@H](C(C)C)C2)CC1]

### Example 2

The synthesis of the title compound was effected analogously to the synthesis of the compound of Example 1 (N-(4-(4-propylpiperazine-1-carbonyl)-3-(6-azaspiro[3.4]octan-6-yl)phenyl) cyclopropanecarboxamide) wherein (3S)-3-propan-2-ylpyrrolidin-1-ium chloride was used instead of 6-aza-spiro[3.4]octane. The title compound was obtained in a yield of 6.5% (7.3 mg).

1H NMR (500 MHz, METHANOL-d4) δ 7.18-7.11 (m, 1 H), 7.06-6.94 (m, 1 H), 6.92-6.85 (m, 1 H), 4.07-3.29 (m, 4 H), 3.37-3.26 (m, 2 H), 3.38-2.90 (m, 2 H), 2.38-2.32 (m, 2 H), 2.71-2.18 (m, 4 H), 2.17-2.07 (m, 1 H), 1.98-1.83 (m, 1 H), 1.79-1.72 (m, 1 H), 1.64-1.47 (m, 4 H), 0.94-0.82 (m, 4 H), 1.05-0.80 (m, 9 H); LC-MS: (m/z) 427.6 (MH+). ps Preparation: N-(3-(1,4-oxazepan-4-yl)-4-(4-propylpiperazine-1-carbonyl)phenyl) cyclopropanecarboxamide

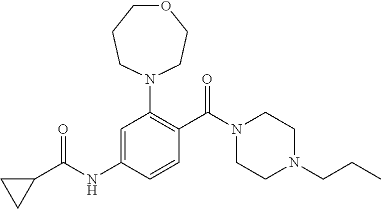 [SMILES: CCCN1CCN(C(=O)c2ccc(NC(=O)C3CC3)cc2N2CCCOCC2)CC1]

### Example 3

N-[3-bromo-4-(4-propylpiperazine-1-carbonyl)phenyl]cyclopropanecarboxamide (50.0 mg, 0.130 mmol) [1-(2-diphenylphosphino-1-naphthalenyl)-2-naphthalenyl]-diphenylphosphine (15.79 mg, 0.030 mmol) and Cs₂CO₃ (123.95 mg, 0.380 mmol) were suspended in dry Toluene (1.5 mL) and the mixture was fluxed with N2 for 10 min. Then [1,4]oxazepane (64.13 mg, 0.630 mmol) and (1E,4E)-1,5-diphenyl-3-penta-1,4-dienone;palladium (11.61 mg, 0.010 mmol) were added and the mixture was heated to 120° C. for 48h. The reaction mixture was cooled down to RT, diluted with water and extracted with AcOEt 3 times. The combined organic fractions were washed with brine, dried over Na₂SO₄, filtered and the solvent removed under reduced pressure. The resulting material was purified by FC on RP using basic conditions (eluting form 100% of ammonium bicarbonate aqueous solution adjusted to pH 10 with ammonia to 100% of CH₃CN 100%) affording the product of formula N-[3-(1,4-oxazepan-4-yl)-4-(4-propylpiperazine-1-carbonyl)phenyl]cyclopropanecarboxamide as white solid.
Yield: 2.2 mg

¹ H NMR (400 MHz, DMSO-d6) δ ppm 10.18 (s, 1 H), 7.37 (d, 1 H), 7.09 (dd, 1 H), 6.96 (d, 1 H), 3.78-3.58 (m, 4 H), 3.61-3.02 (m, 8 H), 2.27-2.20 (m, 2 H), 2.46-2.06 (m, 4 H), 1.85 (quin, 2 H), 1.80-1.71 (m, 1 H), 1.51-1.36 (m, 2 H), 0.84 (t, 3 H), 0.81-0.75 (m, 4 H); LC-MS: m/z 415.3 (MH+) ps Preparation: N-(4-(4-propylpiperazine-1-carbonyl)-3-(7-oxa-2-azaspiro[3.5]nonan-2-yl)phenyl)cyclopropane carboxamide

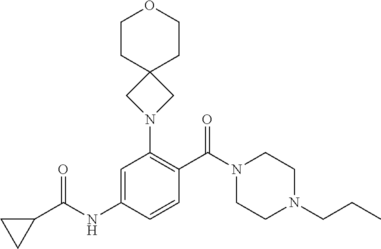 [SMILES: CCCN1CCN(C(=O)c2ccc(NC(=O)C3CC3)cc2N2CC3(CCOCC3)C2)CC1]

### Example 4

The synthesis of the title compound was effected analogously to the synthesis of the compound of Example 1 (N-(4-(4-propylpiperazine-1-carbonyl)-3-(6-azaspiro[3.4]octan-6-yl)phenyl)cyclopropanecarboxamide) wherein 7-oxa-2-azaspiro[3.5]nonane hydrochloride was used instead of 6-aza-spiro[3.4]octane. The title compound was obtained in a yield of 6.5% (7.3 mg). ¹ NMR (500 MHz, DMSO-d6) δ ppm 10.22-10.06 (m, 1 H), 7.00-6.79 (m, 3 H), 3.66-3.47 (m, 10 H), 3.24-3.10 (m, 2 H), 2.46-2.18 (m, 6 H), 1.76 (t, 1 H), 1.72-1.67 (m, 4 H), 1.48-1.38 (m, 2 H), 0.85 (t, 3 H), 0.80-0.75 (m, 4 H); LC-MS: m/z 441.4 (MH+). ps Preparation: N-(3-(3-phenylpyrrolidin-1-yl)-4-(4-propylpiperazine-1-carbonyl)phenyl) cyclopropanecarboxamide

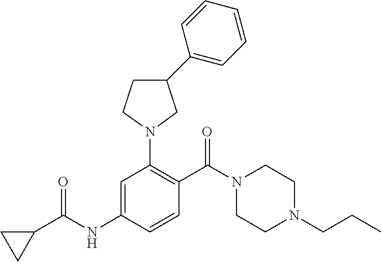 [SMILES: CCCN1CCN(C(=O)c2ccc(NC(=O)C3CC3)cc2N2CCC(c3ccccc3)C2)CC1]

### Example 5

The synthesis of the title compound was effected analogously to the synthesis of the compound of Example 3 (N-(3-(1,4-oxazepan-4-yl)-4-(4-propylpiperazine-1-carbonyl)phenyl)cyclopropanecarboxamide) wherein 3-phenyl-pyrrolidine was used instead of [1,4]oxazepane. The title compound was obtained in a yield of 7.7% (9 mg).

¹ NMR (400 MHz, DMSO-d₆) δ 10.12 (s, 1H), 7.42-7.20 (m, 5H), 7.12 (d, 1H), 6.93 (d, 2H), 3.74-3.10 (m, 4H), 3.28-3.06 (m, 6H), 2.42-2.01 (m, 7H), 1.76 (d, 1H), 1.46-1.38 (m, 2H), 0.93-0.74 (m, 7H); LC-MS: m/z 461.4 (MH+). ps Preparation: N-(4-(4-propylpiperazine-1-carbonyl)-3-(2-oxa-6-azaspiro[3.5]nonan-6-yl)phenyl)cyclopropane carboxamide

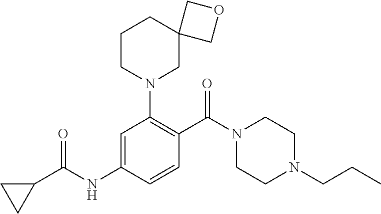 [SMILES: CCCN1CCN(C(=O)c2ccc(NC(=O)C3CC3)cc2N2CCCC3(COC3)C2)CC1]

### Example 6

The synthesis of the title compound was effected analogously to the synthesis of the compound of Example 3 (N-(3-(1,4-oxazepan-4-yl)-4-(4-propylpiperazine-1-carbonyl)phenyl)cyclopropanecarboxamide) wherein 2-oxa-6-azaspiro[3.5]nonane was used instead of [1,4]oxazepane. The title compound was obtained in a yield of 6% (4.8 mg). 1H NMR (400 MHz, DMSO-d6) δ 10.26 (s, 1H), 7.48 (d, 1H), 7.27 (dd, 1H), 7.03 (d, 1H), 4.32 (d, 1H), 4.28-4.21 (m, 3H), 3.71 (d, 1H), 3.49 (d, 1H), 3.17-2.85 (m, 5H), 2.62 (d, 1H), 2.48-2.41 (m, 1H), 2.37-2.21 (m, 4H), 2.16-2.10 (m, 1H), 1.76 (dd, 3H), 1.60-1.51 (m, 2H), 1.42 (q, 2H), 0.91-0.77 (m, 7H); LC-MS: m/z 441.4 (MH+) ps Preparation: N-(3-(4,4-difluoropiperidin-1-yl )-4-(4-propylpiperazine-1-carbonyl)phenyl) cyclopropanecarboxamide

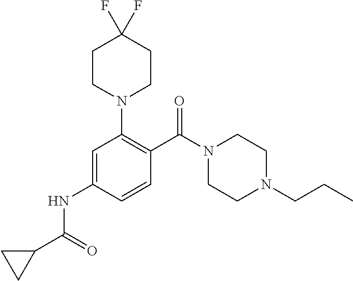 [SMILES: CCCN1CCN(C(=O)c2ccc(NC(=O)C3CC3)cc2N2CCC(F)(F)CC2)CC1]

### Example 7

The synthesis of the title compound was effected analogously to the synthesis of the compound of Example 3 (N-(3-(1,4-oxazepan-4-yl)-4-(4-propylpiperazine-1-carbonyl)phenyl)cyclopropanecarboxamide) wherein 4,4-difluoropiperidine was used instead of [1,4]oxazepane. The title compound was obtained in a yield of 22% (12.3 mg). 1H NMR (500 MHz, DMSO-d6) δ ppm 10.28 (s, 1 H), 7.46 (d, 1 H), 7.25 (dd, 1 H), 7.06 (d, 1 H), 3.75-2.99 (m, 4 H), 3.21-2.85 (m, 4 H), 2.26-2.19 (m, 2 H), 2.45-2.12 (m, 4 H), 2.11-1.95 (m, 4 H), 1.84-1.71 (m, 1 H), 1.42 (sxt, 2 H), 0.84 (t, 3 H), 0.81-0.76 (m, 4 H); LC-MS: m/z 435.3 (MH+). ps Preparation: N-(3-(3-methyl-1H-pyrazol-1-yl)-4-(4-propylpiperazine-1-carbonyl)phenyl)cyclopropanecarboxamide

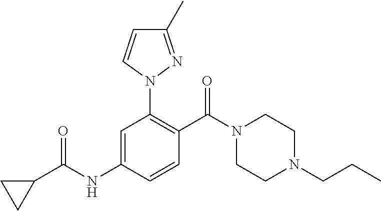 [SMILES: CCCN1CCN(C(=O)c2ccc(NC(=O)C3CC3)cc2-n2ccc(C)n2)CC1]

### Example 8

In a vial were sequentially added N-[3-bromo-4-(4-propylpiperazine-1-carbonyl)phenyl]cyclopropanecarboxamide (100.0 mg, 0.250 mmol), tripotassiumphosphate (114.66 mg, 0.530 mmol), 3-methyl-1H-pyrazole (0.02 mL, 0.300 mmol) and dry Toluene (0.254 mL). The resulting suspension was degassed bubbling N₂ for 15 minutes, then copper (I) iodide (2.43 mg, 0.010 mmol) and N,N′-Dimethylcyclohexane-1,2-diamine (0.0 mL, 0.030 mmol) were added under nitrogen flux and degassed again with Schlenk line technique. The vial was sealed and stirred ON at 120° C. The day after the reaction mixture was diluted with AcOEt and filtered through a pad of celite. The organic layer was evaporated under reduced pressure and purified by FC on RP using basic conditions (eluent: 100% ammonium bicarbonate aqueous solution adjusted to pH 10 with ammonia to CH3CN 100%) then further purified by semi preparative chiral HPLC:

| | | |
|---|---|---|
| | Column | Chiralpak AD-H (25 × 2.0 cm), 5 μ |
| | Mobile phase % v/v | n-Hexane/(Ethanol + 0.1 % |
| | | isopropylamine) 65/35 % v/v |
| | Flow rate (ml/min) | 17 ml/min |
| | DAD detection | 220 nm |
| Preparative chiral | Loop | 1000 μL |
| chromatography | Total amount | 14 mg |
| protocol: | Injection | 14 mg (each injection) |

| | |
|---|---|
| 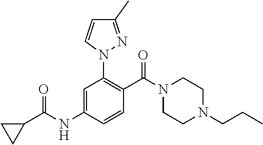 [SMILES: CCCN1CCN(C(=O)c2ccc(NC(=O)C3CC3)cc2-n2ccc(C)n2)CC1] | N-(3-(3-methyl-1H-pyrazol-1-yl)-4-(4-propylpiperazine-1-carbonyl)phenyl) cyclopropanecarboxamide Yield: 6.5 % (6.5 mg) 1H NMR (500 MHz, METHANOL-d4) δ ppm 7.96 (d, 1H), 7.78 (d, 1H), 7.57 (dd, 1H), 7.34 (d, 1H), 6.32 (d, 1H), 3.80-3.66 (m, 1H), 3.64-3.53 (m, 1 H), 3.17 (ddd, 1H), 2.96 (ddd, 1H), 2.55-2.45 (m, 1H), 2.41-2.33 (m, 1H), 2.32 (s, 3H), 2.26 (dd, 3H), 2.00 -1.88 (m, 1H), 1.84 -1.71 (m, 1H), 1.58- 1.41 (m, 2H), 1.02-0.94 (m, 2H), 0.92-0.85 (m, 5H); LC-MS: m/z 396.24 (MH+). |
| | Example 8 |

Preparation: N-[3-(3-methylpyrrol-1-yl)-4-(4-propylpiperazine-1-carbonyl)phenyl] Cyclopropanecarboxamide

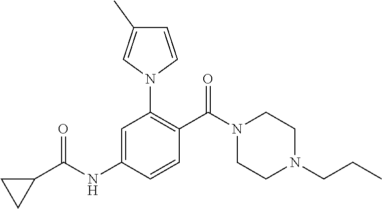 [SMILES: CCCN1CCN(C(=O)c2ccc(NC(=O)C3CC3)cc2-n2ccc(C)c2)CC1]

### Example 9

The synthesis of the title compound was effected analogously to the synthesis of the compound of Example 8 (N-(3-(3-methyl-1H-pyrazol-1-yl)-4-(4-propylpiperazine-1-carbonyl)phenyl) cyclopropanecarboxamide) wherein 3-methyl-1H-pyrrole was used instead of 3-methyl-1H-pyrazole. The title compound was obtained in a yield of 4% (3.6 mg). 1H NMR (500 MHz, DMSO-d6) δ ppm 10.50 (s, 1 H), 7.75 (d, 1 H), 7.48 (dd, 1 H), 7.26 (d, 1 H), 6.80 (t, 1 H), 6.69 (s, 1 H), 6.12-6.02 (m, 1 H), 3.87-2.66 (m, 4 H), 2.11-2.06 (m, 2 H), 2.05 (s, 3 H), 1.79 (quin, 1 H), 2.41-1.37(m, 4 H), 1.36-1.30 (m, 2 H), 0.82 (d, 4 H), 0.79 (t, 3 H); LC-MS: m/z 395.3 (MH+). ps Preparation: N-(6-(4-propylpiperazine-1-carbonyl)-[1,1′-biphenyl]-3-yl)cyclopropanecarboxamide

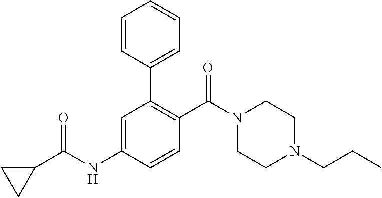 [SMILES: CCCN1CCN(C(=O)c2ccc(NC(=O)C3CC3)cc2-c2ccccc2)CC1]

### Example 10

N-[3-bromo-4-(4-propylpiperazine-1-carbonyl)phenyl]cyclopropanecarboxamide (100.25 mg, 0.250 mmol), phenylboronic acid (31.0 mg, 0.250 mmol), sodium carbonate (188.63 mg, 1.78 mmol) were suspended in a mixture of Toluene (1.112 mL), Ethanol (1.112 mL) and Water (0.318 mL). The resulting suspension was degassed using Schlenk line technique and then palladium tetrakis triphenylphosphine (5.89 mg, 0.010 mmol) was added under nitrogen flux. The mixture was degassed again and heated to 140 ° C. under microwave conditions for 60 minutes. The reaction mixture was cooled down to room temperature, diluted with H₂O and extracted with AcOEt 3 times. The combined organic fractions were washed with Brine, dried over Na₂SO₄, filtered and the solvent removed under vacuum. The raw material was purified by FC on NH column (eluting from cHex/AcOEt 95:5 to 100% of AcOEt) affording the product of formula (N-(6-(4-propylpiperazine-1-carbonyl)-[1,1′-biphenyl]-3-yl)cyclopropanecarboxamide).
Yield: 34 mg

1H NMR (400 MHz, DMSO-d6) δ ppm 10.40 (s, 1 H), 7.74 (d, 1 H), 7.61 (dd, 1 H), 7.51-7.33 (m, 5 H), 7.25 (d, 1 H), 3.63-2.61 (m, 4 H), 2.02-1.97 (m, 2 H), 1.81 (s, 1 H), 1.35-1.24 (m, 2 H), 2.40-1.11 (m, 4 H), 0.92-0.64 (m, 7 H); LC-MS: m/z 392.7 (MH+). ps Preparation: N-(4-(4-propylpiperazine-1-carbonyl)-3-(pyridin-2-yl)phenyl)cyclopropanecarboxamide

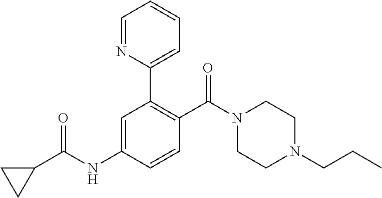 [SMILES: CCCN1CCN(C(=O)c2ccc(NC(=O)C3CC3)cc2-c2ccccn2)CC1]

### Example 11

N-[3-bromo-4-(4-propylpiperazine-1-carbonyl)phenyl]cyclopropanecarboxamide (100.0 mg, 0.250 mmol) was dissolved in dry DMF (1.569 mL), tributyl(2-pyridinyl)stannane (0.1 mL, 0.300 mmol) was added and the solution was degassed with Schlenk line technique for 15 min. Then palladium tetrakis triphenylphosphine (14.51 mg, 0.010 mmol) was added under nitrogen flux and the reaction was stirred ON at 120° C. The day after the reaction mixture was cooled down to RT, diluted with aqueous solution of KF 10% and extracted AcOEt 3 times. The combined organic fractions were then washed with water, brine, dried over Na₂SO₄, filtered and the solvent removed under reduced pressure. The resulting material was purified by FC on NH column (eluting from cHex/AcOEt 95:5 to 100% of AcOEt) then further purified by FC on RP using basic conditions (eluting from 100% of ammonium bicarbonate aqueous solution adjusted to pH 10 with ammonia to CH₃CN 100%) affording the compound of formula (N-[4-(4-propylpiperazine-1-carbonyl)-3-pyridin-2-ylphenyl]cyclopropanecarboxamide).
Yield: 10.5 mg

1H NMR (400 MHz, DMSO-d6) δ ppm 10.44 (s, 1 H), 8.62 (dd, 1 H), 8.00 (d, 1 H), 7.87 (td, 1 H), 7.69 (dd, 1 H), 7.54 (d, 1 H), 7.41-7.36 (m, 1 H), 7.25 (d, 1 H), 3.62-2.75 (m, 4 H), 2.18-2.05 (m, 2 H), 1.88-1.74 (m, 1 H), 2.41-1.58 (m, 4 H), 1.35 (sxt, 2 H), 0.87-0.73 (m, 7 H); LC-MS: m/z 265.3 (MH+).

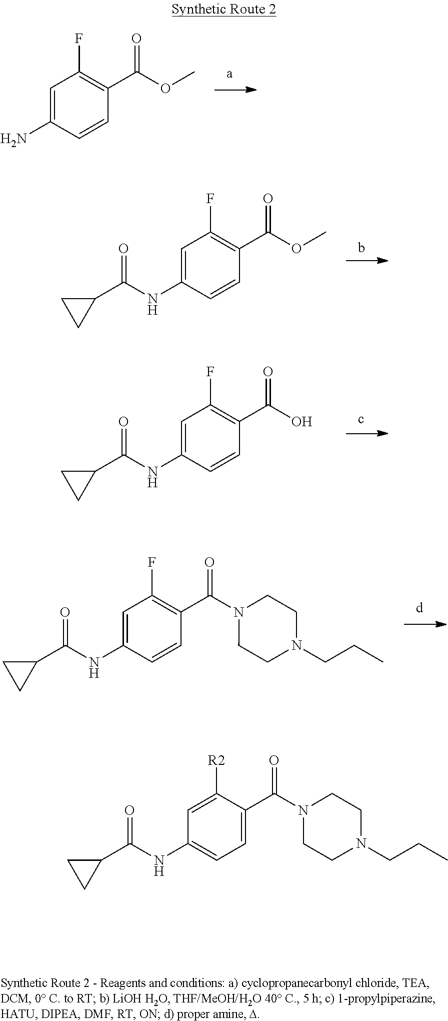

Preparation: N-[3-fluoro-4-(4-propylpiperazine-1-carbonyl)phenyl]cyclopropanecarboxamide

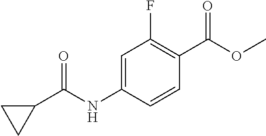 [SMILES: COC(=O)c1ccc(NC(=O)C2CC2)cc1F]

To a cooled solution of methyl 4-amino-2-fluorobenzoate (2.1 g, 12.42 mmol) and trimethylamine (5.19 mL, 37.25 mmol) in DCM (40 mL) at 0° C. a solution of cyclopropanecarbonyl chloride (2.82 mL, 31.04 mmol) in DCM (22 mL) was slowly added. The mixture was warmed to RT and stirred ON. The day after the reaction mixture was diluted with DCM, washed with NaHCO₃, Brine, dried over phase separator and concentrated under reduced pressure. The residue was purified by FC on silica gel (eluting from cHex/AcOEt 95:5 to 3:7) affording the product of formula (methyl 4-(cyclopropanecarbonylamino)-2-fluorobenzoate).
Yield: 1.8 g

¹ NMR (400 MHz, Chloroform-d) δ 7.92 (t, 1H), 7.69-7.60 (m, 1H), 7.23-7.19 (m, 1H), 1.60 (s, 3H), 1.54 (tt, 1H), 1.17-1.11 (m, 2H), 0.93 (dt, 2H); LC-MS: 238.0 (MH+)

Preparation: 4-cyclopropaneamido-2-fluorobenzoic acid

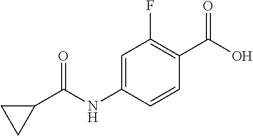 [SMILES: O=C(O)c1ccc(NC(=O)C2CC2)cc1F]

To a solution of methyl 4-(cyclopropanecarbonylamino)-2-fluorobenzoate (1.8 g, 7.72 mmol) in a mixture THF (6.432 mL)/ Methanol (3.216 mL) a solution of lithiumhydroxide (0.37 g, 15.44 mmol) in Water (6.432 mL) was added and the reaction was stirred at 40° C. for 5h. After this time the reaction was cooled down to RT and concentrated under vacuum. The residue was taken up with HCI 1N and the suspension was filtered. The obtained solid was washed with H₂O and dried under vacuum affording the product of formula (4-(cyclopropanecarbonylamino)-2-fluorobenzoic acid).
Yield: 6.9 g

¹ NMR (400 MHz, DMSO-d₆) δ 13.2 (bs, 1H), 10.65 (s, 1H), 7.81 (t, 1H), 7.65 (d, 1H), 7.33 (s, 1H), 1.84-1.73 (m, 1H), 0.92-0.81 (m, 4H); LC-MS: m/z 224.0 (MH+)

Preparation: N-[3-fluoro-4-(4-propylpiperazine-1-carbonyl)phenyl]cyclopropanecarboxamide

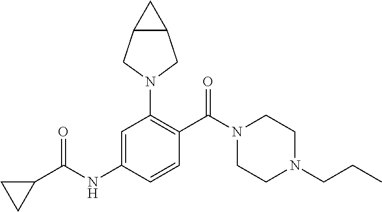 [SMILES: CCCN1CCN(C(=O)c2ccc(NC(=O)C3CC3)cc2N2CC3CC3C2)CC1]

To a solution of 4-(cyclopropanecarbonylamino)-2-fluorobenzoic acid (1.5 g, 6.95 mmol) in DCM (35 mL) 1-hydroxybenzotriazole;hydrate (2.2 g, 14.36 mmol), 3-(ethyliminomethylideneamino)-N,N-dimethyl-1-propanamine hydrochloride (2.2 g, 11.49 mmol) and N,N-Diisopropylethylamine (2.0 mL, 11.49 mmol) were added and the resulting mixture was stirred for 30 minutes at RT. Then a solution of 1-propylpiperazine (1.5 g, 11.49 mmol) in DCM (7mL) was added and the mixture reaction was stirred ON at RT. The day after the mixture diluted with DCM, washed with a s.s. of NaHCO₃, Brine, dried over a phase separator and the solvent removed under reduce pressure. The residue were purified with FC on NH column (eluting from 100% of DCM to DCM/MeOH 95:5) affording the product of formula (N[3-fluoro-4-(4-propylpiperazine-1-carbonyl)phenyl]cyclopropanecarboxamide).
Yield: 1.95 g

¹ NMR (400 MHz, Chloroform-d) δ 7.62 (d, 1H), 7.34 (t, 1H), 7.13 (dd, 1H), 3.83 (d, 2H), 3.37 (s, 2H), 2.52 (t, 2H), 2.46-2.28 (m, 4H), 1.62 (s, 1H), 1.57-1.49 (m, 3H), 1.17-1.09 (m, 2H), 0.97-0.87 (m, 5H), LC-MS: m/z 334.17 (MH+)

Preparation: N-(3-(3-azabicyclo[3.1.0]hexan-3-yl)-4-(4-propylpiperazine-1-carbonyl)phenyl)cyclopropanecarboxamide

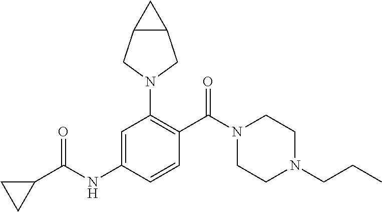 [SMILES: CCCN1CCN(C(=O)c2ccc(NC(=O)C3CC3)cc2N2CC3CC3C2)CC1]

### Example 12

3-Azabicyclo[3.1.0]hexane hydrochloride (358.7 mg, 3 mmol), potassium carbonate (207.27 mg, 1.5 mmol) N-[3-fluoro-4-(4-propylpiperazine-1-carbonyl)phenyl]cyclopropanecarboxamide (100 mg, 0.3 mmol) were mixed in in DMF (0.500 mL) and the reaction was heated to 120° C. for 1 week. Then the reaction was cooled down to RT and purified by FC on RP using basic conditions (eluenting from 100% of ammonium bicarbonate aqueous solution adjusted to pH 10 with ammonia to 100% of CH₃CN) affording the product of formula (N-[3-(3-azabicyclo[3.1.0]hexan-3-y0-4-(4-propylpiperazine-1-carbonyl)phenyl]cyclopropanecarboxamide).
Yield: 5 mg

¹ NMR (400 MHz, DMSO-d6) δ 10.11 (s, 1H), 7.05 (d, 1H), 6.97 (dd, 1H), 6.85 (d, 1H), 3.76-3.66 (m, 1H), 3.51-3.43 (m, 1H), 3.41-3.34 (m, 2H), 3.21-3.09 (m, 4H), 2.46-2.42 (m, 1H), 2.33-2.29 (m, 2H), 2.27-2.22 (m, 2H), 2.21-2.14 (m, 1H), 1.76 (p, 1H), 1.65-1.59 (m, 2H), 1.43 (h, 2H), 0.86 (d, 3H), 0.82-0.75 (m, 4H), 0.62 (td, 1H), 0.26 (q, 1H); LC-MS: m/z 397.6 (MH)+

Preparation: N-(3-(3-methylpyrrolidin-1-yl)-4-(4-propylpiperazine-1-carbonyl)phenyl) cyclopropanecarboxamide

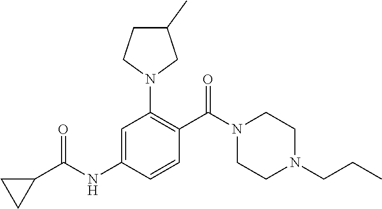 [SMILES: CCCN1CCN(C(=O)c2ccc(NC(=O)C3CC3)cc2N2CCC(C)C2)CC1]

### Examples 13a, 13b

The synthesis of the title compound was effected analogously to the synthesis of the compound of Example 12 (N-(3-(3-azabicyclo[3.1.0]hexan-3-y0-4-(4-propylpiperazine-1-carbonyl)phenyl) cyclopropanecarboxamide) wherein 3-methylpyrrolidine hydrochloride was used instead of 3-Azabicyclo[3.1.0]hexane hydrochloride. The title compound was obtained in a yield of 15% (18 mg).

¹ NMR (500 MHz, METHANOL-d4) δ ppm 7.19-7.10 (m, 1 H), 7.07-6.98 (m, 1 H), 6.93-6.87 (m, 1 H), 4.10-3.12 (m, 7 H), 2.95-2.80 (m, 1 H), 2.51-2.42 (m, 2 H), 2.79-2.29 (m, 4 H), 2.40-2.28 (m, 1 H), 2.15-2.04 (m, 1 H), 1.80-1.71 (m, 1 H), 1.65-1.51 (m, 3 H), 1.18-1.06 (m, 3 H), 1.00-0.80 (m, 7 H); LC-MS: m/z 399.6 (M H) The racemic mixture was then separated into single enantiomers by preparative HPLC

| | |
|---|---|
| Column | Whelk O1 (R,R) (25 × 0.46 cm), 10 |

| | | |
|---|---|---|
| | | um |
| | Mobile phase % v/v | (2-propanol + 0.1% |
| | | isopropylamine) 40% |
| | Flow rate (ml/min) | 2.5 ml/min |
| | DAD detection | 220 nm |
| Preparative chiral | Loop | 25 μL |
| chromatography | Total amount | 18 mg |
| protocol: | Injection | 6.9 mg (each injection) |

| 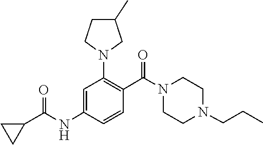 [SMILES: CCCN1CCN(C(=O)c2ccc(NC(=O)C3CC3)cc2N2CCC(C)C2)CC1] | Enantiomer 1 | Rt 11.04 min | 100% ee |
|---|---|---|---|
| 3R or 3S enantiomer | LC-MS: m/z 399.6 (MH+) | | |
| | N-(3-[(3R or 3S] 3-methylpyrrolidin-1-yl]-4-(4-propylpiperazine-1- | | |
| | carbonyl)phenyl) cyclopropanecarboxamide | | |
| | Example 13a | | |
| 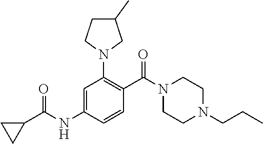 [SMILES: CCCN1CCN(C(=O)c2ccc(NC(=O)C3CC3)cc2N2CCC(C)C2)CC1] | Enantiomer 2 | Rt 13.13 min | 43% ee |

| | |
|---|---|
| 3R or 3S enantiomer | LC-MS: m/z 399.6 (MH+) |
| | N-(3-[(3R or 3S] 3-methylpyrrolidin-1-yl]-4-(4-propylpiperazine-1- |
| | carbonyl)phenyl) cyclopropanecarboxamide |
| | Example 13b |

Preparation: syn/anti N-(3-((-3,5-dimethylpiperidin-1-yl)-4-(4-propylpiperazine-1-carbonyl)phenypcyclopropane carboxamide

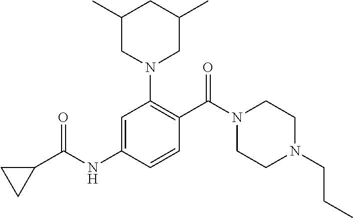 [SMILES: CCCN1CCN(C(=O)c2ccc(NC(=O)C3CC3)cc2N2CC(C)CC(C)C2)CC1]

### Examples 14a and 14b

The synthesis of the title compound was effected analogously to the synthesis of the compound of Example 12 (N-(3-(3-azabicyclo[3.1.0]hexan-3-yl)-4-(4-propylpiperazine-1-carbonyl)phenyl) cyclopropanecarboxamide) wherein 3,5-dimethylpiperidine was used instead of 3-Azabicyclo[3.1.0]hexane hydrochloride. The title compound was obtained as mixture of syn/anti diasteroisomers in a yield of 99% (100 mg). The mixture of syn/anti isomers was then separated into single isomers by preparative chiral HPLC

| | Column | Chiralpak AD-H (25 × 2.0 cm), 5 μum |
|---|---|---|
| | Mobile phase % v/v | (2-propanol + 0.1% isopropylamine) 15% |
| | Flow rate (ml/min) | 45 ml/min |
| | Pressure (bar) | 120 |
| | Temperature | 38 |
| | DAD detection | 220 nm |
| Preparative chiral | Loop | 800 μL |
| chromatography | Total amount | 100 mg |
| protocol: | Injection | 9.9 mg (each injection) |
| 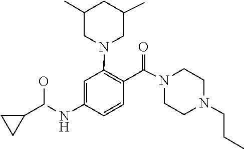 [SMILES: CCCN1CCN(C(=O)c2ccc(NCOCC3CC3)cc2N2CC(C)CC(C)C2)CC1] | Syn isomer | Rt 6.5 min |
| Syn | LC-MS: m/z 427.4 (MH+) | |
| | 1H NMR (500 MHz, DMSO-d6) δ ppm 10.22 (br s, 1H), 7.34 (d, 1H), | |
| | 7.20 (dd, 1H), 7.02 (d, 1H), 3.68 (td, 1H), 3.55-3.43 (m, 1H), 3.27 | |
| | (br dd, 1H), 3.19-2.97 (m, 3H), 2.45-2.28 (m, 3H), 2.25-2.17 (m, | |
| | 3H), 2.11 (ddd, 1H), 1.94 (t, 1H), 1.94 (t, 1H), 1.81-1.62 (m, 4H), 1.48- | |
| | 1.37 (m, 2H), 0.90 (d, 3H), 0.86-0.81 (m, 3H), 0.80-0.75 (m, 7H), | |
| | 0.60 (q, 1H); | |
| | Example 14a | |
| 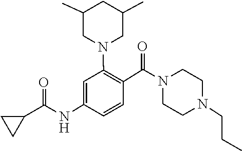 [SMILES: CCCN1CCN(C(=O)c2ccc(NC(=O)C3CC3)cc2N2CC(C)CC(C)C2)CC1] | Anti isomer | Rt 8.2 min |

| | |
|---|---|
| Anti | LC-MS: m/z 427.4 (MH+) |
| | ¹H NMR (400 MHz, DMSO-d6) δ ppm , 10.31-10.14 (m, 1H), 7.45- |
| | 7.33 (m, 1H), 7.27-7.16 (m, 1H), 7.03-6.95 (m, 1H), 3.96-2.81 (m, |
| | 4H), 3.12-2.35 (m, 4H), 2.27-2.19 (m, 2H), 2.47-1.99 (m, 4H), 2.08- |
| | 1.83 (m, 2H), 1.76 (br s, 1H), 1.49-1.37 (m, 2H), 1.35-1.27 (m, 2 |
| | H), 1.00-0.89 (m, 6H), 0.83 (t, 3H), 0.81-0.75(m, 4H) |
| | Example 14b |

Preparation: N-(3-(4-methylpiperidin-1-yl)-4-(4-propylpiperazine-1-carbonyl)phenyl) cyclopropanecarboxamide

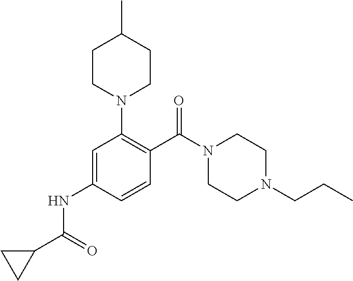 [SMILES: CCCN1CCN(C(=O)c2ccc(NC(=O)C3CC3)cc2N2CCC(C)CC2)CC1]

### Example 15

The synthesis of the title compound was effected analogously to the synthesis of the compound of Example 12 (N-(3-(3-azabicyclo[3.1.0]hexan-3-yl)-4-(4-propylpiperazine-1-carbonyl)phenyl) cyclopropanecarboxamide) wherein 4-methylpiperidine was used instead of 3-Azabicyclo[3.1.0]hexane hydrochloride. The title compound was obtained in a yield of 26% (32 mg). 1H NMR (400 MHz, DMSO-d6) δ ppm 10.21 (s, 1 H), 7.37 (d, 1 H), 7.18 (dd, 1 H), 7.01 (d, 1 H), 3.71-3.03 (m, 4 H), 3.40-2.31 (m, 4 H), 2.28-2.20 (m, 2 H), 2.46-2.05 (m, 4 H), 1.80-1.69 (m, 1 H), 1.43 (sxt, 3 H), 1.89-1.13 (m, 4 H), 0.93 (d, 3 H), 0.84 (t, 3 H), 0.81-0.75 (m, 4 H); LC-MS: m/z 413.3 (MH+).

Preparation: N-[4-(4-propylpiperazine-1-carbonyl)-3-(pyrrolidin-1- yl)phenyl] Cyclopropanecarboxamide

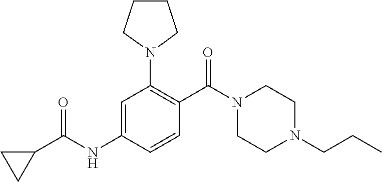 [SMILES: CCCN1CCN(C(=O)c2ccc(NC(=O)C3CC3)cc2N2CCCC2)CC1]

### Example 16

The synthesis of the title compound was effected analogously to the synthesis of the compound of Example 12 (N-(3-(3-azabicyclo[3.1.0]hexan-3-yl)-4-(4-propylpiperazine-1-carbonyl)phenyl) cyclopropanecarboxamide) wherein pyrrolidine was used instead of 3-Azabicyclo[3.1.0]hexane hydrochloride. The title compound was obtained in a yield of 30% (28 mg).

¹ NMR (400 MHz, Chloroform-d) δ 7.39 (s, 1H), 7.18 (s, 1H), 7.06 (d, 1H), 6.66 (dd, 1H), 4.01 (d, 1H), 3.60 (t, 1H), 3.47-3.14 (m, 6H), 2.66-2.17 (m, 6H), 1.93 (t, 4H), 1.52 (p, 3H), 1.11 (p, 2H), 0.94 (d, 3H), 0.91-0.84 (m, 2H); LC-MS: m/z 385.5 (MH+)

Preparation: N-(3-(4,4-dimethylpiperidin-1-yl)-4-(4-propylpiperazine-1-carbonyl)phenyl)cyclopropanecarboxamide

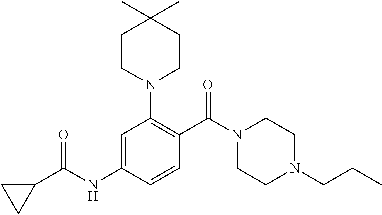 [SMILES: CCCN1CCN(C(=O)c2ccc(NC(=O)C3CC3)cc2N2CCC(C)(C)CC2)CC1]

### Example 17

N-(3-(4-methylpiperidin-1-yl)-4-(4-propylpiperazine-1-carbonyl)phenyl)cyclopropanecarboxamide The synthesis of the title compound was effected analogously to the synthesis of the compound of Example 12 (N-(3-(3-azabicyclo[3.1.0]hexan-3-yl)-4-(4-propylpiperazine-1-carbonyl)phenyl) cyclopropanecarboxamide) wherein 4,4-dimethylpiperidine was used instead of 3-Azabicyclo[3.1.0]hexane hydrochloride. The title compound was obtained in a yield of 24% (37 mg).

¹ NMR (400 MHz, DMSO-d6) δ 10.22 (s, 1 H), 7.44 (d, 1 H), 7.17 (dd, 1 H), 7.01 (d, 1 H), 3.70-3.47 (m, 2 H), 3.20-2.68 (m, 6 H), 2.47-2.14 (m, 6 H), 1.82-1.70 (m, 1 H), 1.51-1.34 (m, 6 H), 0.95 (s, 6 H), 0.84 (t, 3 H), 0.81-0.76 (m, 4 H); LC-MS: m/z 427.3 (MH+).

Preparation: N-(3-(3,4-dihydroisoquinolin-2(1H)-yl)-4-(4-propylpiperazine-1-carbonyl)phenyl)cyclopropane carboxamide

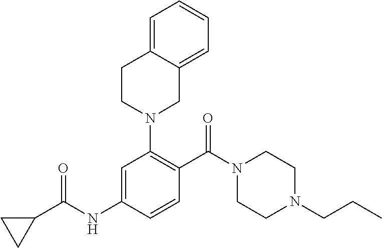 [SMILES: CCCN1CCN(C(=O)c2ccc(NC(=O)C3CC3)cc2N2CCc3ccccc3C2)CC1]

### Example 18

The synthesis of the title compound was effected analogously to the synthesis of the compound of Example 12 (N-(3-(3-azabicyclo[3.1.0]hexan-3-yl)-4-(4-propylpiperazine-1-carbonyl)phenyl) cyclopropanecarboxamide) wherein 1,2,3,4-tetrahydroisoquinoline was used instead of 3-Azabicyclo[3.1.0]hexane hydrochloride. The title compound was obtained in a yield of 12% (8 mg).

¹ NMR (400 MHz, DMSO-d6) δ ppm 10.28 (s, 1 H), 7.49 (d, 1 H), 7.24 (dd, 1 H), 7.20-7.03 (m, 5 H), 4.25 (d, 1 H), 4.01 (d, 1 H), 3.36 (br d, 2 H), 3.66-3.04 (m, 4 H), 2.94-2.78 (m, 2 H), 2.36-2.06 (m, 4 H), 2.02 (t, 2 H), 1.84-1.72 (m, 1 H), 1.35-1.19 (m, 2 H), 0.89-0.62 (m, 7 H); LC-MS: m/z 447.3 (MH+).

Preparation: N-(3-(piperidin-1-yl)-4-(4-propylpiperazine-1-carbonyl)phenyl)cyclopropanecarboxamide

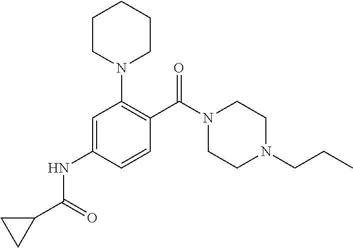 [SMILES: CCCN1CCN(C(=O)c2ccc(NC(=O)C3CC3)cc2N2CCCCC2)CC1]

### Example 19

The synthesis of the title compound was effected analogously to the synthesis of the compound of Example 12 (N-(3-(3-azabicyclo[3.1.0]hexan-3-yl)-4-(4-propylpiperazine-1-carbonyl)phenyl) cyclopropanecarboxamide) wherein piperidine was used instead of 3-Azabicyclo[3.1.0]hexane hydrochloride. The title compound was obtained in a yield of 41% (50 mg). 1H NMR (400 MHz, DMSO-d6) δ ppm, 10.22 (s, 1 H), 7.37 (d, 1 H), 7.19 (dd, 1 H), 7.02 (d, 1 H), 3.82-2.93 (m, 4 H), 3.07-2.68 (m, 4 H), 2.28-2.20 (m, 2 H), 2.47-2.03 (m, 4 H), 1.81-1.71 (m, 1 H), 1.69-1.46 (m, 6 H), 1.46-1.37 (m, 2 H), 0.84 (t, 3 H), 0.81-0.74 (m, 4 H); LC-MS: m/z 399.3 (MH+).

Preparation: N-[3-(cyclopentylamino)-4-(4-propylpiperazine-1-carbonyl)phenyl]cyclopropanecarboxamide

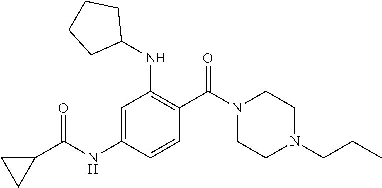 [SMILES: CCCN1CCN(C(=O)c2ccc(NC(=O)C3CC3)cc2NC2CCCC2)CC1]

### Example 20

The synthesis of the title compound was effected analogously to the synthesis of the compound of Example 12 (N-(3-(3-azabicyclo[3.1.0]hexan-3-yl)-4-(4-propylpiperazine-1-carbonyl)phenyl) cyclopropanecarboxamide) wherein cyclopentanamine was used instead of 3-Azabicyclo[3.1.0]hexane hydrochloride. The title compound was obtained in a yield of 26% (23mg).

¹ NMR (400 MHz, DMSO-d₆) δ 10.12 (s, 1H), 7.10 (d, 1H), 6.96 (d, 1H), 6.84 (dd, 1H), 5.38 (d, 1H), 3.70-3.60 (m, 1H), 3.51-3.41 (m, 4H), 2.34 (t, 4H), 2.25 (d, 2H), 1.97 (dt, 2H), 1.82-1.75 (m, 1H), 1.72-1.54 (m, 4H), 1.50-1.34 (m, 4H), 0.86 (t, 3H), 0.81-0.74 (m, 4H); LC-MS: m/z 399.50 (MH+).

X₂ groups or moieties bridged through a carbonyl linker may be prepared using Synthetic Scheme 3.

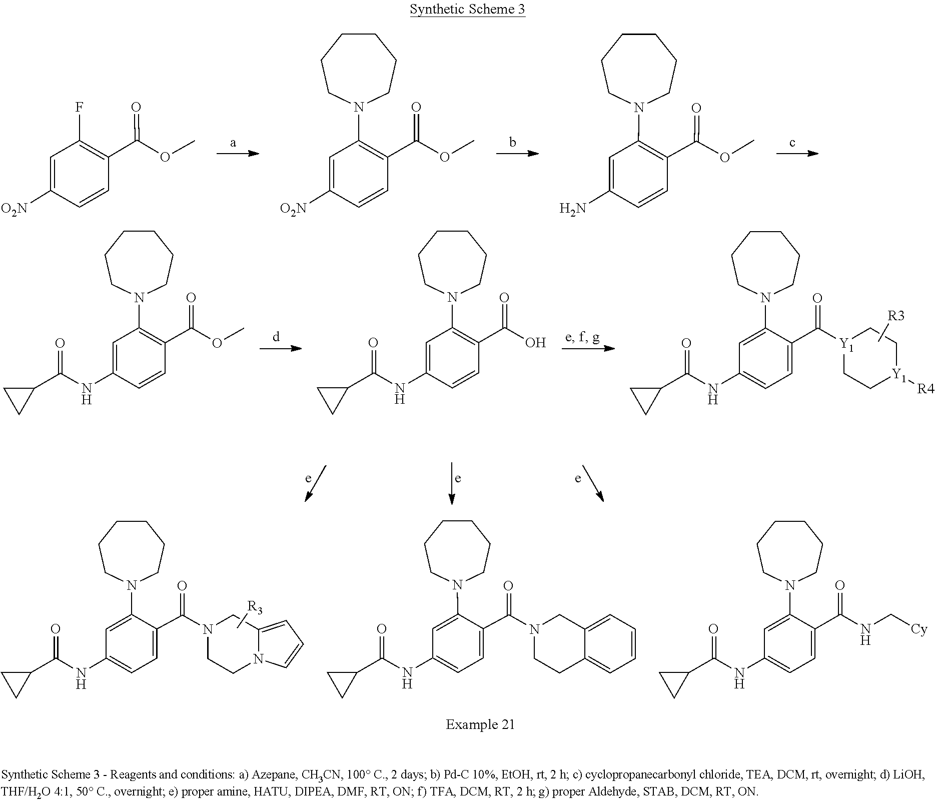

Preparation: methyl 2-(3,5-dimethylpiperidin-1-yl)-4-nitrobenzoate

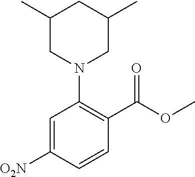

A mixture of methyl 2-fluoro-4-nitrobenzoate (3.0 g, 15.06 mmol) and 3,5-dimethylpiperidine (6.0 mL, 45.19 mmol) in MeCN (25.11 mL) was stirred at RT for 2h. After this time the reaction mixture was concentrate under reduced pressure. The residue was taken up with AcOEt, washed with water and Brine.

The organic phase was dried over Na₂SO₄, filtered and concentrated under reduced pressure. The residue was purified by FC on silica gel (eluenting from 100% of cHex to cHex/AcOEt 7:3) affording the product of formula methyl 2-(3,5-dimethylpiperidin-1-yl)-4-nitrobenzoate as mixture as syn/anti isomers. The product was used as such in the next step.
Yield: 4 g
LC-MS: m/z 293.1 (MH+)
   Preparation: methyl 2-(azepan-1-yl)-4-nitrobenzoate

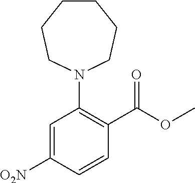 [SMILES: COC(=O)c1ccc([N+](=O)[O-])cc1N1CCCCCC1]

The synthesis of the title compound was effected analogously to the synthesis of methyl 2-(3,5-dimethylpiperidin-1-yl)-4-nitrobenzoate wherein Homopiperidine was used instead of 3,5-dimethylpiperidine. The title compound was obtained in a yield of 93% (2.6g).

¹ NMR (400 MHz, Chloroform-d) δ 7.78 (d, 1H), 7.62 (d, 1H), 7.51 (dd, 1H), 3.93 (s, 3H), 3.42-3.36 (m, 4H), 1.90— 1.79 (m, 4H), 1.63 (t, 4H); LC-MS: m/z 278.7 (MH+).

Preparation: methyl 4-nitro-2-pyrrolidin-1-ylbenzoate

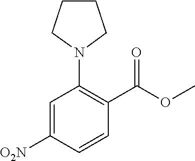 [SMILES: COC(=O)c1ccc([N+](=O)[O-])cc1N1CCCC1]

The synthesis of the title compound was effected analogously to the synthesis of methyl 2-(3,5-dimethylpiperidin-1-yl)-4-nitrobenzoate wherein pyrrolidine was used instead of 3,5-dimethylpiperidine. The title compound was obtained in a yield of 94% (5.9 g).

1H NMR (400 MHz, DMSO-d6) δ 7.64 (d, 1H), 7.49 (d, 1H), 7.44 (dd, 1H), 3.86 (s, 3H), 3.26-3.20 (m, 4H), 1.95-1.90 (m, 4H); LC-MS: 251.05 (MH+).

Preparation: methyl 2-(6-azaspiro[3.4]octan-6-yl)-4-nitrobenzoate

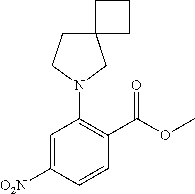 [SMILES: COC(=O)c1ccc([N+](=O)[O-])cc1N1CCC2(CCC2)C1]

To a solution of methyl 2-fluoro-4-nitrobenzoate (600.0 mg, 3.01 mmol) and N,N-Diisopropylethylamine (1.05 mL, 6.03 mmol) in DMF (8 mL), 6-aza-spro[3.4]octane (502.47 mg, 4.52 mmol) was added and the reaction was stirred at 80° C. overnight. UPLC The reaction was cooled down to RT and water was added. The mixture was extracted with AcOEt 3 times. Combined organics were washed with Brine, dried over a phase separator and concentrated under vacuum. The crude was purified by FC on silica gel (eluenting from 100% of cHex to cHex/AcOEt 9:1) affording the product of formula methyl 2-(6-azaspiro[3.4]octan-6-yl)-4-nitrobenzoate
Yield: 792 mg

1H NMR (400 MHz, DMSO-d6) δ 7.64 (d, 1H), 7.51-7.36 (m, 2H), 3.86 (s, 3H), 3.24 (t, 2H), 3.18 (s, 2H), 2.08-1.74 (m, 8H); LC-MS: m/z 291.1 (MH+).

Preparation: methyl 2-(3-methylpyrrolidin-1-yl)-4-nitrobenzoate

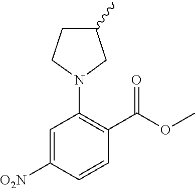 [SMILES: COC(=O)c1ccc([N+](=O)[O-])cc1N1CCC(C)C1]

The synthesis of the title compound was effected analogously to the synthesis of methyl 2-(3,5-dimethylpiperidin-1-yl)-4-nitrobenzoate wherein 3-methylpyrrolidine hydrochloride and trimethylamine were used instead of 6-aza-spro[3.4]octane and N,N-Diisopropylethylamine respectively. The title compound was obtained in a yield of 92% (2.45 g). 1H NMR (400 MHz, DMSO-d6) δ 7.64 (d, 1H), 7.47 (d, 1H), 7.43 (dd, 1H), 3.86 (s, 3H), 3.38-3.19 (m, 3H), 2.88 (dd, 1H), 2.31 (dq, 1H), 2.07 (dtd, 1H), 1.64-1.49 (m, 1H), 1.06 (d, 3H); LC-MS: m/z 265.28 (MH+).

Preparation: methyl 4-amino-2-(3,5-dimethylpiperidin-1-yl)benzoate

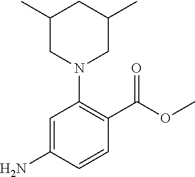 [SMILES: COC(=O)c1ccc(N)cc1N1CC(C)CC(C)C1]

To a solution of methyl 2-(3,5-dimethylpiperidin-1-yl)-4-nitrobenzoate (4.0 g, 13.68 mmol) in Ethanol (54.73 mL) palladium (1456.16 mg, 1.37 mmol) was added and the reaction was stirred at RT for 2 h under H2 atmosphere. After this time the mixture was filtered through a pad of celite washing with AcOEt and solution was concentrated under vacuum affording the product of formula methyl 4-amino-2-(3,5-dimethylpiperidin-1-yl)benzoate as mixture of syn/ant isomers. The crude material was used in the next step without further purification.
Yield: 3.5 g
LC-MS: m/z 264.1 (MH+)
   Preparation: methyl 4-amino-2-pyrrolidin-1-ylbenzoate

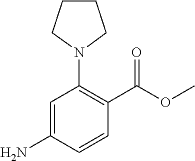 [SMILES: COC(=O)c1ccc(N)cc1N1CCCC1]

The synthesis of the title compound was effected analogously to the synthesis of methyl 4-amino-2-(3,5-dimethylpiperidin-1-yl)benzoate wherein methyl 4-nitro-2-pyrrolidin-1-ylbenzoate was used instead of methyl 2-(3,5-dimethylpiperidin-1-yl)-4-nitrobenzoate. The title compound was obtained in a yield of 92% (5 g).

1H NMR (400 MHz, DMSO-d6) δ 7.29 (d, 1H), 6.02-5.88 (m, 2H), 5.47 (s, 2H), 3.67 (s, 3H), 3.16-2.99 (m, 4H), 2.00-1.71 (m, 4H); LC-MS: m/z 221.39 (MH+).

Preparation: methyl 4-amino-2-(6-azaspiro[3.4]octan-6-yObenzoate

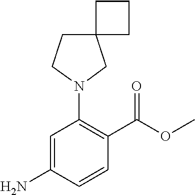 [SMILES: COC(=O)c1ccc(N)cc1N1CCC2(CCC2)C1]

The synthesis of the title compound was effected analogously to the synthesis of methyl 4-amino-2-(3,5-dimethylpiperidin-1-yl)benzoate wherein methyl 2-(6-azaspiro[3.4]octan-6-yl)-4-nitrobenzoate was used instead of methyl 2-(3,5-dimethylpiperidin-1-yl)-4-nitrobenzoate. The title compound was obtained in a yield of 95% (672 mg). 1H NMR (400 MHz, DMSO-d6) δ 7.28 (d, 1H), 5.98-5.87 (m, 2H), 5.47 (s, 2H), 3.67 (s, 3H), 3.10 (t, 2H), 3.04 (s, 2H), 2.01-1.74 (m, 8H); LC-MS: m/z 261.1 (MH+).

Preparation methyl 4-amino-2-(3-methylpyrrolidin-1-yl)benzoate

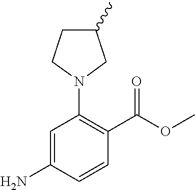 [SMILES: COC(=O)c1ccc(N)cc1N1CCC(C)C1]

The synthesis of the title compound was effected analogously to the synthesis of methyl 4-amino-2-(3,5-dimethylpiperidin-1-yl)benzoate wherein methyl methyl 2-(3-methylpyrrolidin-1-yl)-4-nitrobenzoate was used instead of methyl 2-(3,5-dimethylpiperidin-1-yl)-4-nitrobenzoate. The title compound was obtained in a yield of 99% (1.5 g).

¹ NMR (400 MHz, DMSO-d6) δ 7.28 (d, 1H), 6.00-5.88 (m, 2H), 5.46 (s, 2H), 3.67 (s, 3H), 3.25 (td, 1H), 3.14-2.97(m, 2H), 2.80 (dd, 1H), 2.28-2.14 (m, 1H), 1.98 (dtd, 1H), 1.53-1.40 (m, 1H), 1.03 (d, 3H); LC-MS: m/z 245.72 (MH+)

Preparation: methyl 4-amino-2-(azepan-1-yl)benzoate

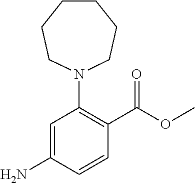 [SMILES: COC(=O)c1ccc(N)cc1N1CCCCCC1]

A mixture of methyl 2-(azepan-1-yl)-4-nitrobenzoate (6.3 g, 22.18 mmol) and 10% palladium on carbon (2.36 g, 2.22 mmol) in Ethanol (100 mL) was stirred under H2 atmosphere at RT overnight. The day after the reaction was filtered through a pad of celite and concentrated under vacuum affording the product of formula methyl 4-amino-2-(azepan-1-yl)benzoate.
Yield: 5.4 g

¹ NMR (400 MHz, Chloroform-d) δ 7.53 (d, 1H), 6.22 (d, 1H), 6.10 (dd, 1H), 3.84 (s, 3H), 3.39-3.29 (m, 4H), 1.83-1.71 (m, 4H), 1.67-1.55 (m, 6H); LC-MS: m/z 249.1 (MH+)

Preparation: methyl 4-(cyclopropanecarbonylamino)-2-(3,5-dimethylpiperidin-1-yl)benzoate

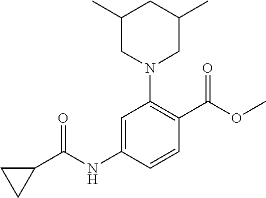 [SMILES: COC(=O)c1ccc(NC(=O)C2CC2)cc1N1CC(C)CC(C)C1]

A solution of methyl 4-amino-2-(3,5-dimethylpiperidin-1-yl)benzoate (3.5 g, 13.34 mmol) and N,N-Diisopropylethylamine (5.58 mL, 40.02 mmol) in DCM (66.7 mL) was cooled down to 0° C. and cyclopropanecarbonyl chloride (2.42 mL, 26.68 mmol) was added dropwise. The mixture was stirred at RT/ON. The day after the reaction mixture was dilute with DCM and washed with s. s. of NaHCO₃, Brine, dried over Na₂SO₄, filtered and concentrate under reduced pressure affording the formula product methyl 4-(cyclopropanecarbonylamino)-2-(3,5-dimethylpiperidin-1-yl)benzoate as mixture of syn/anti isomers, which was used in the next step without further purification.
Yield: 4.2 g
LC-MS: m/z 331.15 (MH+).
   Preparation: methyl 4-(cyclopropanecarbonylamino)-2-pyrrolidin-1-ylbenzoate

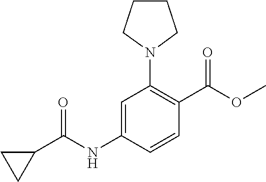 [SMILES: COC(=O)c1ccc(NC(=O)C2CC2)cc1N1CCCC1]

The synthesis of the title compound was effected analogously to the synthesis of methyl 4-(cyclopropanecarbonylamino)-2-(3,5-dimethylpiperidin-1-yl)benzoate wherein methyl 4-amino-2-pyrrolidin-1-ylbenzoate was used instead of methyl 4-amino-2-(3,5-dimethylpiperidin-1-yl)benzoate. The title compound was obtained in a yield of 99% (6 g). 1H NMR (400 MHz, DMSO-d6) δ 10.22 (s, 1H), 7.41 (d, 1H), 7.22 (d, 1H), 6.92 (dd, 1H), 3.75 (s, 3H), 3.11 (q, 4H), 1.92-1.83 (m, 4H), 1.83-1.75 (m, 1H), 0.83-0.76 (m, 4H); LC-MS: m/z 289.12 (MH+).

Preparation: methyl 2-(6-azaspiro[3.4]octan-6-yl)-4-(cyclopropanecarbonylamino)benzoate

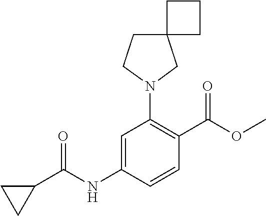 [SMILES: COC(=O)c1ccc(NC(=O)C2CC2)cc1N1CCC2(CCC2)C1]

The synthesis of the title compound was effected analogously to the synthesis of methyl 4-(cyclopropanecarbonylamino)-2-(3,5-dimethylpiperidin-1-yl)benzoate wherein methyl 4-amino-2-(6-azaspiro[3.4]octan-6-yl)benzoate was used instead of methyl 4-amino-2-(3,5-dimethylpiperidin-1-yl)benzoate. The title compound was obtained in a yield of 88% (742 mg).

¹ NMR (400 MHz, DMSO-d6) δ 10.21 (s, 1H), 7.41 (d, 1H), 7.22 (d, 1H), 6.87 (dd, 1H), 3.75 (s, 3H), 3.12 (t, 2H), 3.06 (s, 2H), 2.04-1.70 (m, 9H), 0.87-0.73 (m, 4H); LC-MS: m/z 329.11 (MH+).

Preparation: methyl 4-(cyclopropanecarbonylamino)-2-(3-methylpyrrolidin-1-yl)benzoate

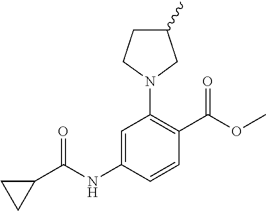 [SMILES: COC(=O)c1ccc(NC(=O)C2CC2)cc1N1CCC(C)C1]

The synthesis of the title compound was effected analogously to the synthesis of methyl 4-(cyclopropanecarbonylamino)-2-(3,5-dimethylpiperidin-1-yl)benzoate wherein methyl 4-amino-2-(3-methylpyrrolidin-1-yl)benzoate was used instead of methyl 4-amino-2-(3,5-dimethylpiperidin-1-yl)benzoate. The title compound was obtained in a yield of 99% (1.7 g). LC-MS: m/z 303.05 (MH+)

Preparation: methyl 2-(azepan-1-yl)-4-cyclopropaneamidobenzoate

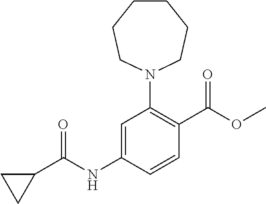 [SMILES: COC(=O)c1ccc(NC(=O)C2CC2)cc1N1CCCCCC1]

A solution of methyl 4-amino-2-(azepan-1-yl)benzoate (5.4 g, 21.75 mmol) and N,N-Diisopropylethylamine (7.58 mL, 43.49 mmol) in DCM (45 mL) was cooled to 0° C. and after 5 minutes cyclopropanecarbonyl chloride (2.96 mL, 32.62 mmol) was added dropwise. The reaction was stirred at RT ON. The day after the reaction was diluted with DCM and washed with NaHCO3 s.s.and Brine. The organic phase was dried over Na₂SO₄, filtered and concentrated under vacuum. The residue was purified by FC on NH column (eluting from cHex/AcOEt 95:5 to cHex/AcOEt 50:50) affording the product of formula methyl 2-(azepan-1-yl)-4-(cyclopropanecarbonylamino)benzoate.
Yield: 6.5 g.

¹ NMR (400 MHz, Chloroform-d) δ 7.55 (d, 2H), 7.45 (s, 1H), 6.66 (dd, 1H), 3.87 (s, 3H), 3.38-3.32 (m, 4H), 1.83-1.75 (m, 4H), 1.60 (h, 4H), 1.58-1.43 (m, 1H), 1.17-1.09 (m, 2H), 0.87 (dt, 2H); LC-MS: m/z 317.2 (MH+).

Preparation: 2-(6-azaspiro[3.4]octan-6-y0-4-(cyclopropanecarbonylamino)benzoic acid

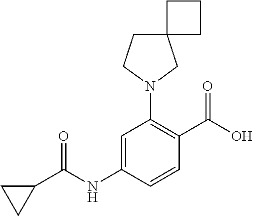 [SMILES: O=C(O)c1ccc(NC(=O)C2CC2)cc1N1CCC2(CCC2)C1]

To a solution of methyl 2-(6-azaspiro[3.4]octan-6-y0-4-(cyclopropanecarbonylamino)benzoate (742.0 mg, 2.26 mmol) in a mixture of THF (16 mL)/Methanol (19 mL) a solution of lithium hydroxide (974 mg, 40.68 mmol) in Water (8 mL) was added. The reaction was stirred at 80° C. for 24h. After this time the reaction was cooled down to RT and a formation of precipitate was observed. The suspension was filtered and mother liquor was concentrated under vacuum. The residue was taken up with H₂O, washed with DCM 3 times. The aqueous phase was acidified until pH=1 with HCI 3 N and extracted with AcOEt 3 times. The organic phase was washed with Brine, dried over Na₂SO₄, filtered and concentrated under vacuum affording the product of formula 2-(6-azaspiro[3.4]octan-6-y0-4-(cyclopropanecarbonylamino)benzoic acid.
Yield: 330 mg

1H NMR (400 MHz, DMSO-d6) δ 12.68 (s, 1H), 10.23 (s, 1H), 7.49 (d, 1H), 7.30 (d, 1H), 6.92 (dd, 1H), 3.23-3.13 (m, 2H), 3.11 (s, 2H), 2.05-1.67 (m, 9H), 0.88-0.68 (m, 4H); LC-MS: m/z 315.16 (MH+).

Preparation: 4-(cyclopropanecarbonylamino)-2-(3-methylpyrrolidin-1-yl)benzoic acid

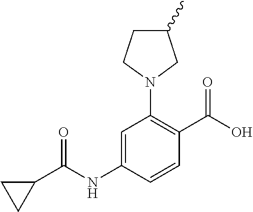 [SMILES: CC1CCN(c2cc(NC(=O)C3CC3)ccc2C(=O)O)C1]

To a solution of methyl 4-(cyclopropanecarbonylamino)-2-(3-methylpyrrolidin-1-yl)benzoate (2.46 g, 5.86 mmol) in a mixture of THF (11 mL)/Methanol (4 mL) a solution of lithium hydroxide (1.5 g, 62.63 mmol) Water (4 mL) was added and the reaction mixture was stirred for 60 hours at 50° C. The reaction was cooled down to RT, acidified until pH =1 with HCI 3N and concentrated under vacuum. The residue was directly purified by FC on RP using acidic conditions (eluting from CH₃CN/H₂O 5:95 +0.1% of formic acid to CH₃CN/H₂O 7:3 +0.1% of formic acid) affording the product of formula 4-(cyclopropanecarbonylamino)-2-(3-methylpyrrolidin-1-yl)benzoic acid.
Yield: 1.06 g

1H NMR (400 MHz, DMSO-d6) δ 12.82 (s, 1H), 10.22 (s, 1H), 7.49 (d, 1H), 7.30 (d, 1H), 6.94 (dd, 1H), 3.30 (td, 2H), 3.20-3.12 (m, 1H), 3.10 (ddd, 1H), 2.27 (dt, 1H), 2.10-1.98 (m, 1H), 1.78 (tt, 1H), 1.52 (dq, 1H), 1.06 (d, 3H), 0.86-0.73 (m, 4H); LC-MS: m/z 289.12 (MH+).

Preparation: 4-(cyclopropanecarbonylamino)-2-pyrrolidin-1-ylbenzoic acid

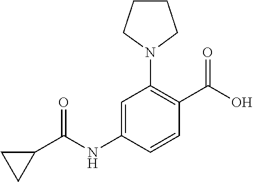 [SMILES: O=C(O)c1ccc(NC(=O)C2CC2)cc1N1CCCC1]

The synthesis of the title compound was effected analogously to the synthesis of 4-(cyclopropanecarbonylamino)-2-(3-methylpyrrolidin-1-yl)benzoic acid wherein methyl 4-(cyclopropanecarbonylamino)-2-pyrrol idin-1-ylbenzoate was used instead of methyl 4-(cyclopropanecarbonylamino)-2-(3-methylpyrrolidin-1-yl)benzoate. The title compound was obtained in a yield of 49% (3 g).

1H NMR (400 MHz, DMSO-d6) δ 13.10 (s, 1H), 10.25 (s, 1H), 7.52 (d, 1H), 7.34 (d, 1H), 6.99 (dd, 1H), 3.21-3.04 (m, 4H), 2.01-1.78 (m, 4H), 1.79 (tt, 1H), 0.86-0.73 (m, 4H); LC-MS: m/z 275.08 (MH+).

Preparation: syn/anti 4-(cyclopropanecarbonylamino)-2-(3,5-dimethylpiperidin-1-yl)benzoic acid

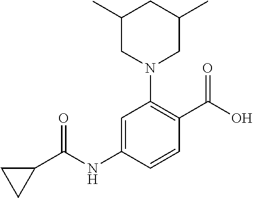 [SMILES: CC1CC(C)CN(c2cc(NC(=O)C3CC3)ccc2C(=O)O)C1]

To a solution of methyl 4-(cyclopropanecarbonylamino)-2-(3,5-dimethylpiperidin-1-yl)benzoate (2.6 g, 7.87 mmol) in a mixture of THF (9.44 mL)/Methanol (3.14 mL) a solution of lithium hydroxide (392.81 mg, 15.74 mmol) in Water (3.14 mL) was added. The resulting mixture was heated to 50° C. ON. The day after the reaction was cooled down to RT, brought to pH 2 with HCI 3N and concentrated under reduced pressure. The obtained solid was taken up with H₂O, filtered, washed with H₂O and dried under vacuum affording the product of formula 4-(cyclopropanecarbonylamino)-2-(3,5-dimethylpiperidin-1-yl)benzoic acid as mixture of syn/anti isomers.
Yield: 2.37 g
LC-MS: m/z 317.19 (MH+).
   Preparation: SYN-4-(cyclopropanecarbonylamino)-2-(3,5-dimethylpiperidin-1-yl)benzoic acid and ANTI- 4-(cyclobrobanecarbonylamino)-2-(3,5-dimethylbiberidin-1-yl)benzoic acid

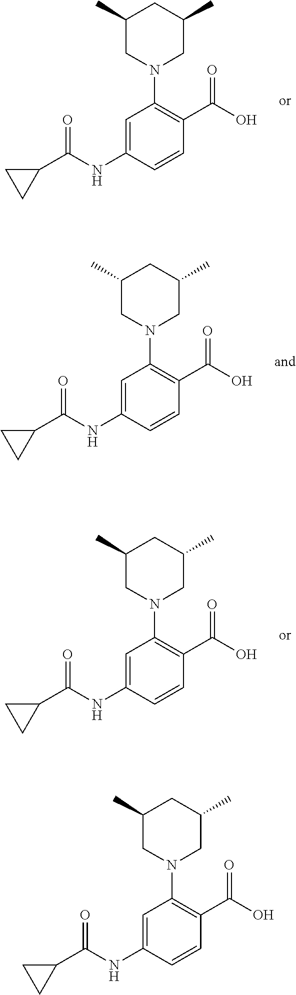 [SMILES: C[C@@H]1C[C@H](C)CN(c2cc(NC(=O)C3CC3)ccc2C(=O)O)C1 ; C[C@@H]1C[C@H](C)CN(c2cc(NC(=O)C3CC3)ccc2C(=O)O)C1 ; C[C@H]1C[C@H](C)CN(c2cc(NC(=O)C3CC3)ccc2C(=O)O)C1 ; C[C@H]1C[C@H](C)CN(c2cc(NC(=O)C3CC3)ccc2C(=O)O)C1]

To a solution of methyl 4-(cyclopropanecarbonylamino)-2-(3,5-dimethylpiperidin-1-yl)benzoate (2.6 g, 7.87 mmol) in a mixture of THF (9.44 mL)/Methanol (3.14 mL) a solution of lithium hydroxide (392.81 mg, 15.74 mmol) in Water (3.14 mL) was added. The resulting mixture was heated to 50° C. ON. The day after the reaction was cooled down to RT, brought to pH 2 with HCI 3N and concentrated under reduced pressure. The obtained solid was taken up with H₂O, filtered, washed with H₂O and dried under vacuum affording the product of formula 4-(cyclopropanecarbonylamino)-2-(3,5-dimethylpiperidin-1-yl)benzoic acid as mixture of syn/anti isomers [2.37 g, LC-MS: m/z 317.19 (MH+)]. The mixture of isomers was then separated into single isomer by Semi preparative MDAP method

| | | |
|---|---|---|
| | Column: | XSelect CSH Prep. C18 5 pm OBD 30 × 100 mm @ room T |
| | Solvents: | A = H₂O + 0.1% HCOOH B = Acetonitrile |
| | Method: | from 30 % B to 44 % B in 10 min |
| Preparative achiral | Injection loop: | 1 mL (each injection) |
| chromatography | Flow rate (ml/min): | 40 ml/min |
| protocol: | UV detection range: | 210 nm to 350 nm |

| | |
|---|---|
| 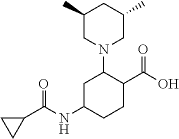 or 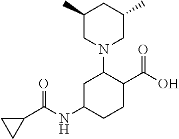 Anti isomer | First eluted compound, Isomer 1: ANTI-4-(cyclopropanecarbonylamino)-2- (3,5-dimethylpiperidin-1-yl)benzoic acid Yield: 7.6 % (188.7mg) 1H NMR (500 MHz, DMSO-d6) δ ppm 10.26 (s, 1H), 7.48 (d, 1H), 7.26 (dd, 1H), 7.07 (d, 1H), 6.72 (s, 1H), 3.91-3.75 (m, 1H), 3.62-3.41 (m, 3H), 2.72- 2.62 (m, 4H), 2.48-2.42 (m, 2H), 2.33 (s, 3H), 1.83-1.67 (m, 3H), 1.28- 1.21 (m, 2H), 0.85-0.76 (m, 4H), 0.71 (d, 6H), LC-MS: m/z 409.9 (MH+) |
| 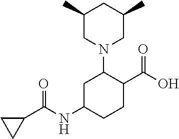 or 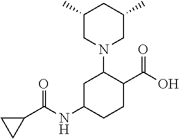 Syn isomer | Second eluted compound, Isomer 2: SYN-4-(cyclopropanecarbonylamino)-2- (3,5-dimethylpiperidin-1-yl)benzoic acid Yield: 46% (1.14 g) 1H NMR (500 MHz, CHLOROFORM-d) δ ppm 8.48 (br s, 1H), 8.38 (d, 1H), 8.19 (d, 1H), 7.11 (dd,H), 2.97-3.09 (m, 2H), 2.53 (td, 2H), 2.09-1.93 (m, 2H), 1.89 (br d, 1H), 1.78-1.68 (m, 1H), 1.16-1.09 (m, 2H), 0.99-0.89 (m, 8H), 0.86-0.73 (m, 1H); LC-MS: m/z 317.11 (MH+). |

Preparation: 2-(azepan-1-yl )-4-cyclopropaneamidobenzoic acid hydrochloride

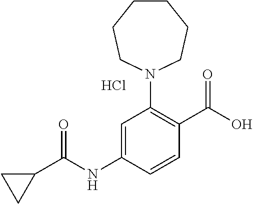 [SMILES: O=C(O)c1ccc(NC(=O)C2CC2)cc1N1CCCCCC1 ; Cl]

To a solution of methyl 2-(azepan-1-yl )-4-(cyclopropanecarbonylamino)benzoate (6.5 g, 20.54 mmol) in THF (24 mL) and Methanol (8 mL) a solution of lithium hydroxide (0.98 g, 41.09 mmol) in Water (8 mL) was added. The reaction was stirred at 50 C ON. The day after the reaction was cooled down to RT and concentrated under vacuum. The residue was taken up with HCI 1 N and stirred at 0 C for 10 min. After this time a solid was observed, therefore the mixture was filtered and the solid washed with H₂O and dried under vacuum affording the product of formula 2-(azepan-1-yl)-4-(cyclopropanecarbonylamino)benzoic acid hydrochloride.
Yield: 6.45 g

¹ NMR (400 MHz, Chloroform-d) δ 10.04 (s, 1H), 8.51 (d, 1H), 8.07 (d, 1H), 7.64 (dd, 1H), 3.49 (s, 4H), 2.07-2.02 (m, 7H), 1.86 (s, 4H), 1.14— 1.06 (m, 2H), 0.94 (dt, 2H). LC-MS: m/z 303.2 (MH+)

Preparation: tert-butyl 4-[2-(azepan-1-yl)-4-cyclopropaneamidobenzoyl]piperazine-1-carboxylate

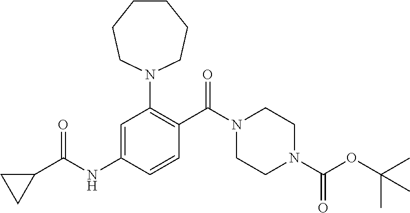 [SMILES: CC(C)(C)OC(=O)N1CCN(C(=O)c2ccc(NC(=O)C3CC3)cc2N2CCCCCC2)CC1]

To a mixture of 2-(azepan-1-yl)-4-(cyclopropanecarbonylamino)benzoic acid hydrochloride (1.0 g, 2.95 mmol), 1-hydroxybenzotriazole;hydrate (0.93 g, 6.09 mmol) and 3-(ethyliminomethylideneamino)-N,N-dimethyl-1-propanamine;hydrochloride (0.93 g, 4.87 mmol) in DCM (20 mL) N,N-Diisopropylethylamine (1.03 mL, 5.9 mmol) was added and the reaction was stirred at RT for 30 min. After this time 1-piperazinecarboxylic acid tert-butyl ester (0.91 g, 4.87 mmol) was added and the reaction was stirred at RT ON. The day after the reaction was diluted with DCM and washed with a s.s. of NaHCO₃, Brine, dried over Na₂SO₄ filtered and concentrated under vacuum. The residue was purified by FC on silica gel (eluting from cHex/AcOEt 9:1 to cHex/AcOEt 1:1) affording the product of formula tert-butyl 4-[2-(azepan-1-yl)-4-(cyclopropanecarbonylamino)benzoyl]piperazine-1-carboxylate.
Yield: 1.18 g

¹ NMR (400 MHz, Chloroform-d) δ 7.53-7.49 (m, 1H), 7.40-7.35 (m, 1H), 7.12 (d, 1H), 6.74 (dd, 1H), 4.04-3.94 (m, 1H), 3.68-3.60 (m, 1H), 3.57-3.49 (m, 4H), 3.42-3.10 (m, 6H), 1.53 (d, 18H), 1.12 (h, 2H), 1.00-0.84 (m, 2H); LC-MS: m/z 471.3 (MH+).

Preparation: N-[3-(azepan-1-yl)-4-(piperazine-1-carbonyl)phenyl]cyclopropanecarboxamide

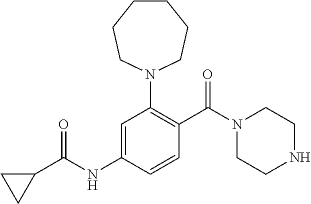 [SMILES: O=C(Nc1ccc(C(=O)N2CCNCC2)c(N2CCCCCC2)c1)C1CC1]

To a solution of tert-butyl 4-[2-(azepan-1-yl)-4-(cyclopropanecarbonylamino)benzoyl]piperazine-1-carboxylate (1.18 g, 2.51 mmol) in DCM (12.54 mL) Trifluoroacetic acid (0.96 mL, 12.54 mmol) was added. The reaction was stirred at RT for 2 h. After this time the mixture was concentrated under vacuum and the residue was purified by SCX first washing with MeOH and then with NH₃ 1M in MeOH affording the product of formula (N-[3-(azepan-1-yl)-4-(piperazine-1-carbonyl)phenyl]cyclopropanecarboxamide).
Yield: 0.8 g

¹ NMR (400 MHz, Chloroform-d) δ 7.59-7.44 (m, 2H), 7.10 (d, 1H), 6.86-6.63 (m, 1H), 4.02 (dt, 1H), 3.56-3.47 (m, 1H), 3.37-3.19 (m, 6H), 3.00-2.87 (m, 2H), 2.80-2.63 (m, 2H), 1.81- 1.73 (m, 4H), 1.61-1.47 (m, 5H), 1.16-1.05 (m, 2H), 0.91-0.81 (m, 2H). LC-MS: m/z 371.3 (MH+)

Preparation: N-(3-(azepan-1-yl )-4-(4-(isoxazol-3-ylmethyl)piperazine carbonyl)phenyl)cyclopropanecarboxamide

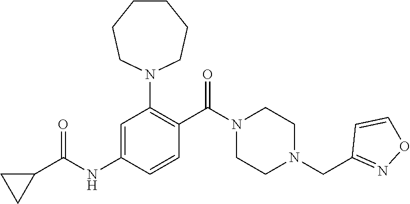 [SMILES: O=C(Nc1ccc(C(=O)N2CCN(Cc3ccon3)CC2)c(N2CCCCCC2)c1)C1CC1]

### Example 22

N-[3-(azepan-1-yl )-4-(piperazine-1-carbonyl)phenyl]cyclopropanecarboxamide (50.0 mg, 0.130 mmol) was suspended in MeCN (3 mL), N,N-Diisopropylethylamine (0.05 mL, 0.270 mmol) was added followed by 1,2-oxazolo-3-carbaldehyde (14.41 mg, 0.150 mmol). The reaction mixture was stirred for 15 min before adding sodium triacetoxyborohydride (57.21 mg, 0.270 mmol). The resulting mixture was stirred at room temperature overnight, then the mixture was diluted with EtOAc and H₂O and the organic phase was separated. The aqueous phase was back-extracted twice with AcOEt, then the combined organic fractions were washed with Brine, dried over Na₂SO₄, filtered and concentrated under vacuum. The residue was purified by FC on NH column (eluting from 100% cHex to 100% EtOAc) affording the formula product N-[3-(azepan-1-yl)-4-[4-(1,2-oxazol-3-ylmethyppiperazine-1-carbonyl]phenyl] cyclopropanecarboxamide.
Yield: 20 mg.

¹ NMR (400 MHz, DMSO-d₆) δ 10.13 (s, 1H), 8.87 (d, 1H), 7.34 (d, 1H), 7.01 (dd, 1H), 6.91 (d, 1H), 6.55 (d, 1H), 3.73-3.54 (m, 4H), 3.18 (td, 6H), 2.46-2.23 (m, 4H), 1.83-1.40 (m, 9H), 0.86-0.63 (m, 4H). LC-MS: m/z 452.4 (MH+)

Preparation: N-[3-(azepan-1-yl)-4-{4-[(1H-pyrazol-3-yOmethyl]piperazine-1-carbonyl}phenyl] cyclopropanecarboxamide

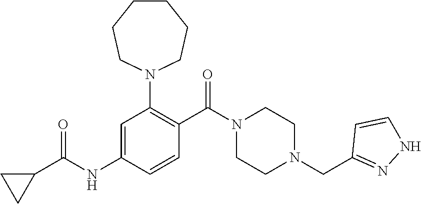 [SMILES: O=C(Nc1ccc(C(=O)N2CCN(Cc3cc[nH]n3)CC2)c(N2CCCCCC2)c1)C1CC1]

### Example 23

The synthesis of the title compound was effected analogously to the synthesis of the compound of Example 22 (N-(3-(azepan-1-yl)-4-(4-(isoxazol-3-ylmethyl)piperazine-1-carbonyl)phenyl) cyclopropanecarboxamide) wherein pyrazole-3-carbaldehyde was used instead of 1,2-oxazolo-3-carbaldehyde. The title compound was obtained in a yield of 21% (13mg). ¹ NMR (400 MHz, DMSO-d₆) δ 12.62 (d, 1H), 10.13 (s, 1H), 7.33 (d, 2H), 7.01 (dd, 1H), 6.90 (d, 1H), 6.14 (s, 1H), 3.66-3.48 (m, 4H), 3.21-3.11 (m, 6H), 2.45-2.16 (m, 4H), 1.87-1.32 (m, 9H), 0.83-0.67 (m, 4H). LC-MS: m/z 451.4 (MH+).

Preparation: N-(3-(azepan-1-yl)-4-(4-(oxazol-2-ylmethyl)piperazine-1-carbonyl)phenyl) cyclopropanecarboxamide

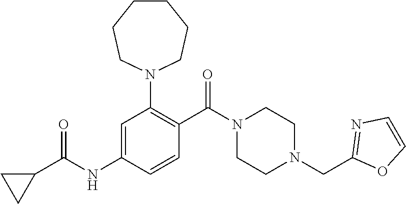 [SMILES: O=C(Nc1ccc(C(=O)N2CCN(Cc3ncco3)CC2)c(N2CCCCCC2)c1)C1CC1]

### Example 24

The synthesis of the title compound was effected analogously to the synthesis of the compound of Example 22 (N-(3-(azepan-1-yl)-4-(4-(isoxazol-3-ylmethyl)piperazine-1-carbonyl)phenyl) cyclopropanecarboxamide) wherein 2-oxazolecarboxaldehyde was used instead of 1,2-oxazolo-3-carbaldehyde. The title compound was obtained in a yield of 37% (22.5 mg).

¹ NMR (400 MHz, DMSO-d₆) δ 10.13 (s, 1H), 8.08 (d, 1H), 7.34 (d, 1H), 7.18 (d, 1H), 7.01 (dd, 1H), 6.91 (d, 1H), 3.72 (s, 2H), 3.59 (d, 2H), 3.24-3.08 (m, 6H), 2.42-2.31 (m, 4H), 1.79-1.41 (m, 9H), 0.83-0.74 (m, 4H); LC-MS: m/z 452.4 (MH+).

Preparation: N-(3-(azepan-1-yl)-4-(4-(3-methoxybenzyl)piperazine-1-carbonyl)phenyl) Cyclopropanecarboxamide

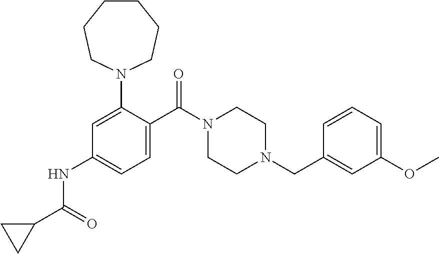 [SMILES: COc1cccc(CN2CCN(C(=O)c3ccc(NC(=O)C4CC4)cc3N3CCCCCC3)CC2)c1]

### Example 25

The synthesis of the title compound was effected analogously to the synthesis of the compound of Example 22 (N-(3-(azepan-1-yl)-4-(4-(isoxazol-3-ylmethyl)piperazine-1-carbonyl)phenyl) cyclopropanecarboxamide) wherein 3-methoxybenzaldehyde was used instead of 1,2-oxazolo-3-carbaldehyde. The title compound was obtained in a yield of 46% (30.5 mg). 1H NMR (400 MHz, DMSO-d6) δ ppm 10.12 (s, 1 H), 7.32 (d, 1 H), 7.23 (t, 1 H), 7.06-6.97 (m, 1 H), 6.94-6.77 (m, 4 H), 3.73 (s, 3 H), 3.47 (d, 4 H), 3.25-3.08 (m, 6 H), 2.45-2.17 (m, 4 H), 1.80-1.43 (m, 9 H) 0.82-0.71 (m, 4 H); LC-MS: m/z 491.3 (MH+).

Preparation: N-(3-(azepan-1-yl)-4-(4-(3-fluorobenzyl)piperazine-1-carbonyl)phenyl) Cyclopropanecarboxamide

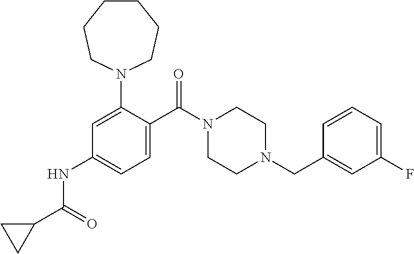 [SMILES: O=C(Nc1ccc(C(=O)N2CCN(Cc3cccc(F)c3)CC2)c(N2CCCCCC2)c1)C1CC1]

### Example 26

The synthesis of the title compound was effected analogously to the synthesis of the compound of Example 22 (N-(3-(azepan-1-yl )-4-(4-(isoxazol-3-ylmethyl)piperazine-1-carbonyl)phenyl) cyclopropanecarboxamide) wherein 3-fluorobenzaldehyde was used instead of 1,2-oxazolo carbaldehyde. The title compound was obtained in a yield of 40% (25 mg). 1H NMR (400 MHz, DMSO-d6) δ 10.13 (s, 1 H), 7.33 (d, 2 H), 7.18-6.98 (m, 4 H), 6.91 (d, 1 H), 3.53 (d, 4 H), 3.27-3.10 (m, 6 H), 2.46-2.19 (m, 4 H), 1.80-1.45 (m, 9 H), 0.84-0.72 (m, 4 H); LCMS: m/z 479.3 (MH+).

Preparation: N-(3-(azepan-1-yl)-4-(4-(2-methoxybenzyl)piperazine-1-carbonyl)phenyl) cyclopropanecarboxamide

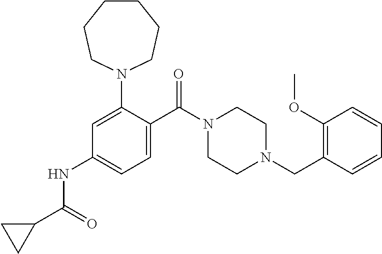 [SMILES: COc1ccccc1CN1CCN(C(=O)c2ccc(NC(=O)C3CC3)cc2N2CCCCCC2)CC1]

### Example 27

The synthesis of the title compound was effected analogously to the synthesis of the compound of Example 22 (N-(3-(azepan-1-yl)-4-(4-(isoxazol-3-ylmethyl)piperazine-1-carbonyl)phenyl) cyclopropanecarboxamide) wherein 2-methoxybenzaldehyde was used instead of 1,2-oxazolo-3-carbaldehyde. The title compound was obtained in a yield of 59% (58 mg). 1H NMR (400 MHz, DMSO-d6) δ 10.19-10.05 (m, 1 H), 7.37-7.19 (m, 3 H), 6.90 (d, 4 H), 3.75 (s, 3 H), 3.62-3.49 (m, 4 H), 3.25-3.09 (m, 6 H), 2.46-2.20 (m, 4 H), 1.75-1.49 (m, 9 H), 0.83-0.71 (m, 4 H); LC-MS: m/z 491.3 (MH+).

Preparation: N-(3-(azepan-1-yl)-4-(4-(2-fluorobenzyl)piperazine-1-carbonyl)phenyl) Cyclopropanecarboxamide

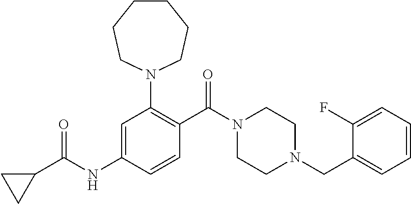 [SMILES: O=C(Nc1ccc(C(=O)N2CCN(Cc3ccccc3F)CC2)c(N2CCCCCC2)c1)C1CC1]

### Example 28

The synthesis of the title compound was effected analogously to the synthesis of the compound of Example 22 (N-(3-(azepan-1-yl)-4-(4-(isoxazol-3-ylmethyl)piperazine-1-carbonyl)phenyl) cyclopropanecarboxamide) wherein 2-fluorobenzaldehyde was used instead of 1,2-oxazolo-3-carbaldehyde. The title compound was obtained in a yield of 42% (41 mg). 1H NMR (400 MHz, DMSO-d6) δ ppm 10.16-10.04 (m, 1 H), 7.43-7.36 (m, 1 H), 7.32 (d, 2 H), 7.20-7.13 (m, 2 H), 7.03-6.97 (m, 1 H), 6.90 (d, 1 H), 3.57 (s, 4 H), 3.23-3.05 (m, 6 H), 2.45-2.21 (m, 4 H), 1.78 -1.43 (m, 9 H), 0.82-0.73 (m, 4 H); LC-MS: m/z 479.3 (MH+).

Preparation: N-(3-(azepan-1-yl)-4-(4-(4-fluorobenzyl)piperazine-1-carbonyl)phenyl) Cyclopropanecarboxamide

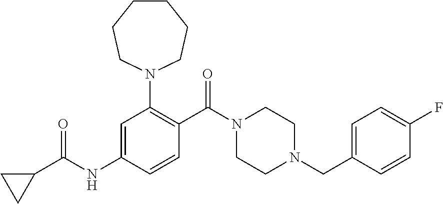 [SMILES: O=C(Nc1ccc(C(=O)N2CCN(Cc3ccc(F)cc3)CC2)c(N2CCCCCC2)c1)C1CC1]

### Example 29

The synthesis of the title compound was effected analogously to the synthesis of the compound of Example 22 (N-(3-(azepan-1-yl)-4-(4-(isoxazol-3-ylmethyl)piperazine-1-carbonyl)phenyl) cyclopropanecarboxamide) wherein 4-fluorobenzaldehyde was used instead of 1,2-oxazolo-3-carbaldehyde. The title compound was obtained in a yield of 65% (56 mg).

1H NMR (400 MHz, DMSO-d6) δ ppm 10.12 (s, 1 H), 7.39-7.27 (m, 3 H), 7.19-7.08 (m, 2 H), 7.00 (dd, 1 H), 6.90 (d, 1 H), 3.52-3.43 (m, 2 H), 3.25-3.06 (m, 4 H), 3.70-3.06 (m, 4 H), 2.46-2.16 (m, 4 H), 1.81-1.71 (m, 1 H), 1.70-1.43 (m, 8 H), 0.85-0.69 (m, 4 H); LC-MS: m/z 479.5 (MH+).

Preparation: N-(3-(azepan-1-yl)-4-(4-(4-methoxybenzyl)piperazine-1-carbonyl)phenyl) cyclopropanecarboxamide

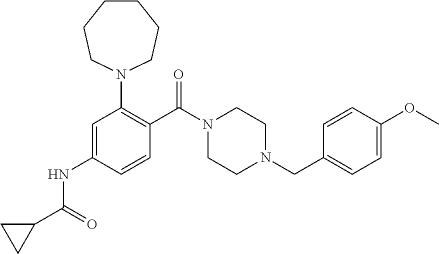 [SMILES: COc1ccc(CN2CCN(C(=O)c3ccc(NC(=O)C4CC4)cc3N3CCCCCC3)CC2)cc1]

### Example 30

The synthesis of the title compound was effected analogously to the synthesis of the compound of Example 22 (N-(3-(azepan-1-yl)-4-(4-(isoxazol-3-ylmethyl)piperazine-1-carbonyl)phenyl) cyclopropanecarboxamide) wherein 4-methoxybenzaldehyde was used instead of 1,2-oxazolo-3-carbaldehyde. The title compound was obtained in a yield of 56% (50 mg). 1H NMR (400 MHz, DMSO-d6) δ ppm , 10.12 (br s, 1 H), 7.32 (d, 1 H), 7.19 (d, 2 H), 7.00 (dd, 1 H), 6.88 (dd, 3 H), 3.73 (s, 3 H), 3.67-3.47(m, 2 H), 3.42 (d, 2 H), 3.24-3.06 (m, 6 H), 2.43-2.16 (m, 4 H), 1.82-1.71 (m, 1 H), 1.69-1.40 (m, 8 H), 0.88-0.70 (m, 4 H); LC-MS: m/z 491.5 (MH+).

Preparation: N-(3-(azepan-1-yl)-4-(4-(2-chlorobenzyl)piperazine-1-carbonyl)phenyl) Cyclopropanecarboxamide

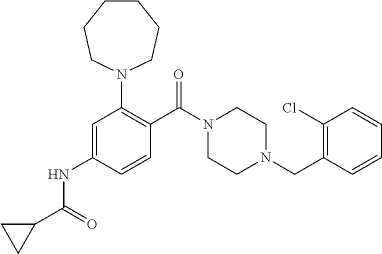 [SMILES: O=C(Nc1ccc(C(=O)N2CCN(Cc3ccccc3Cl)CC2)c(N2CCCCCC2)c1)C1CC1]

### Example 31

The synthesis of the title compound was effected analogously to the synthesis of the compound of Example 22 (N-(3-(azepan-1-yl )-4-(4-(isoxazol-3-ylmethyppiperazine-1-carbonypphenyl) cyclopropanecarboxamide) wherein 2-chlorobenzaldehyde was used instead of 1,2-oxazolo carbaldehyde. The title compound was obtained in a yield of 66% (52 mg). 1H NMR (400 MHz, DMSO-d6) δ ppm 10.13 (s, 1 H), 7.49 (dd, 1 H), 7.43 (dd, 1 H), 7.43 (dd, 1 H), 7.37 -7.24 (m, 3 H), 7.04-6.97 (m, 1 H), 6.92 (d, 1 H), 3.26-3.09 (m, 4 H), 3.74-3.07 (m, 4 H), 2.49-2.25 (m, 4 H), 1.83-1.45 (m, 9 H), 0.85-0.71 (m, 4 H); LC-MS: m/z 495.4 (MH+).

Preparation: N-(3-(azepan-1-yl)-4-(4-(pyridin-3-ylmethyl)piperazine-1-carbonyl)phenyl)cyclopropanecarboxamide

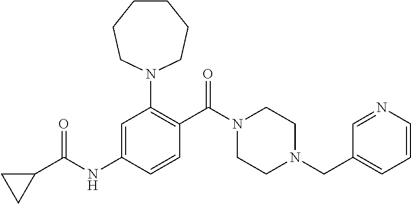 [SMILES: O=C(Nc1ccc(C(=O)N2CCN(Cc3cccnc3)CC2)c(N2CCCCCC2)c1)C1CC1]

### Example 32

The synthesis of the title compound was effected analogously to the synthesis of the compound of Example 22 (N-(3-(azepan-1-yl)-4-(4-(isoxazol-3-ylmethyl)piperazine-1-carbonyl)phenyl) cyclopropanecarboxamide) wherein 3-pyridinecarboxaldehyde was used instead of 1,2-oxazolo-3-carbaldehyde. The title compound was obtained in a yield of 34% (25 mg). 1H NMR (400 MHz, DMSO-d6) δ ppm 10.12 (s, 1 H), 8.49 (d, 1 H), 8.47 (dd, 1 H), 7.71 (dt, 1 H), 7.35 (dd, 1 H), 7.32 (d, 1 H), 7.00 (dd, 1 H), 6.91 (d, 1 H), 3.53 (br d, 2 H), 3.27-3.06 (m, 4 H), 3.72-3.06 (m, 4 H), 2.46-2.16 (m, 4 H), 1.80-1.71 (m, 1 H), 1.73-1.41 (m, 8 H), 0.85-0.69 (m, 4 H); LC-MS: m/z 462.4 (MH+).

Preparation: N-[3-(azepan-1-yl)-4-(4-thieno[2,3-c]pyridin-7-ylpiperazine-1-carbonyl)phenyl]Cyclopropanecarboxamide

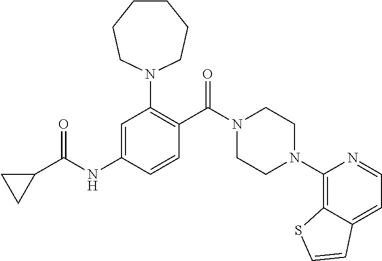 [SMILES: O=C(Nc1ccc(C(=O)N2CCN(c3nccc4ccsc34)CC2)c(N2CCCCCC2)c1)C1CC1]

### Example 33

To a solution of N-[3-(azepan-1-yl )-4-(piperazine-1-carbonyl)phenyl]cyclopropanecarboxamide (75.0 mg, 0.200 mmol) and N,N-Diisopropylethylamine (0.05 mL, 0.300 mmol) in DMSO (2 mL), 7-chlorothieno[2,3-C]pyridine (41.21 mg, 0.240 mmol) was added and the reaction was stirred at 120° C. overnight and then heated to 150° C. for 2 hrs under microwave conditions. The reaction was concentrated to dryness and the residue was taken up with AcOEt. The organic phase was washed with water, Brine, dried over Na₂SO₄, filtered and concentrated under vacuum. The residue was purified by FC on silica gel (eluting from 100% of AcOEt to AcOEt/MeOH 8:2) affording the formula product N-[3-(azepan-1-yl)-4-(4-thieno[2,3-c]pyridin-7-ylpiperazine-1-carbonyl)phenyl]Cyclopropanecarboxamide.
Yield: 9 mg

1H NMR (400 MHz, DMSO-d6) δ 10.11-10.19 (m, 1 H) 8.09 (d, 1 H) 8.01 (d, 1 H) 7.49 (d, 1 H) 7.39 (d, 2 H) 7.01 (s, 2 H) 3.82-3.90 (m, 1 H) 3.34-3.80 (m, 7 H) 3.21-3.27 (m, 4 H) 1.45-1.81 (m, 9 H) 0.80 (d, 4 H); LC-MS: m/z 504.4 (MH+).

Preparation: N-[3-(azepan-1-yl)-4-(4-thieno[3,2-d]pyrimidin-4-ylpiperazine-1-carbonyl)phenyl]Cyclopropanecarboxamide

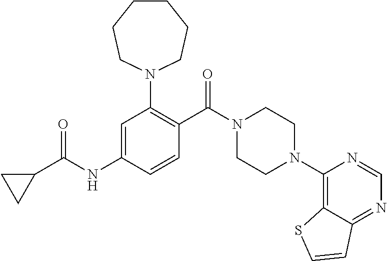 [SMILES: O=C(Nc1ccc(C(=O)N2CCN(c3ncnc4ccsc34)CC2)c(N2CCCCCC2)c1)C1CC1]

### Example 34

The synthesis of the title compound was effected analogously to the synthesis of the compound of Example 33 (N-[3-(azepan-1-yl)-4-(4-thieno[2,3-c]pyridin-7-ylpiperazine-1-carbonyl)phenyl]Cyclopropanecarboxamide) wherein 4-chlorothieno[2,3-D]pyrimidine was used instead of 7-chlorothieno[2,3-C]pyridine. The title compound was obtained in a yield of 85% (86 mg). 1H NMR (400 MHz, DMSO-d6) δ 10.13-10.19 (m, 1 H) 8.51 (s, 1 H) 8.23 (d, 1 H) 7.41-7.48 (m, 1 H) 7.33-7.40 (m, 1 H) 6.96-7.07 (m, 2 H) 3.65-4.14 (m, 6 H) 3.32-3.45 (m, 2 H) 3.16-3.26 (m, 4 H) 1.43-1.82 (m, 9 H) 0.74-0.85 (m, 4 H); LC-MS: m/z 505.4 (MH+).

Preparation: N-(3-(azepan-1-yl)-4-(piperidine-1-carbonyl)phenyl)cyclopropanecarboxamide

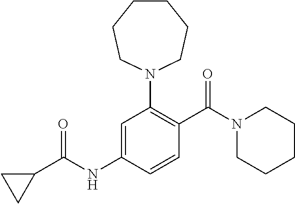 [SMILES: O=C(Nc1ccc(C(=O)N2CCCCC2)c(N2CCCCCC2)c1)C1CC1]

### Example 35

2-(azepan-1-yl)-4-(cyclopropanecarbonylamino)benzoic acid hydrochloride (50.0 mg, 0.150 mmol), HATU: [dimethylamino(3-triazolo[4,5-b]pyridinyloxy)methylidene]-dimethylammonium;hexafluorophosphate (67.33 mg, 0.180 mmol) and N,N-Diisopropylethylamine (76.29 mg, 0.590 mmol) were mixed in DMF (1.5 mL) and stirred for 5 min, then piperidine (14.45 mg, 0.170 mmol) was added. The reaction mixture was stirred at RT for 2. After this time the reaction was concentrated under vacuum and the residue was purified by SCX cartridge first washing with MeOH and then eluting with NH₃ 1M in MeOH. The basic fractions were evaporated and the residue was purified by FC on NH column (eluting from 100% of cHex to cHex/AcOEt 70:30) affording the product of formula N-[3-(azepan-1-yl)-4-(piperidine-1-carbonyl)phenyl]cyclopropanecarboxamide.
Yield: 46 mg

¹ NMR (400 MHz, DMSO-d₆) δ 10.11 (s, 1H), 7.32 (d, 1H), 7.01 (dd, 1H), 6.90 (d, 1H), 3.80-3.38 (m, 2H), 3.25-3.10 (m, 6H), 1.82-1.25 (m, 15H), 0.84-0.73 (m, 4H); LC-MS: m/z 370.3 (MH+).

Preparation: N-(3-(azepan-1-yl)-4-(4-propylpiperazine-1-carbonyl)phenyl)cyclopropanecarboxamide

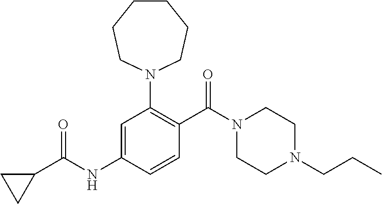 [SMILES: CCCN1CCN(C(=O)c2ccc(NC(=O)C3CC3)cc2N2CCCCCC2)CC1]

### Example 36

The synthesis of the title compound was effected analogously to the synthesis of the compound of Example 35 (N-(3-(azepan-1-yl )-4-(piperidine-1-carbonyl)phenyl)cyclopropanecarboxamide) wherein azepane was used instead of piperidine. The title compound was obtained in a yield of 77.2% (470 mg). NMR: ¹H NMR (400 MHz, DMSO-d₆) δ 10.13 (s, 1H), 7.34 (d, 1H), 7.01 (dd, 1H), 6.92 (s, 1H), 3.70-3.62 (m, 1H), 3.55-3.46 (m, 1H), 3.26-3.08 (m, 6H), 2.45-2.12 (m, 6H), 1.81-1.36 (m, 11H), 0.85 (t, 3H), 0.80-0.76 (m, 4H); LC-MS: m/z 413.3 (MH+).

Preparation: N-[3-(azepan-1-yl)-4-(2,4-dimethylpiperazine-1-carbonyl)phenyl]cyclopropanecarboxamide

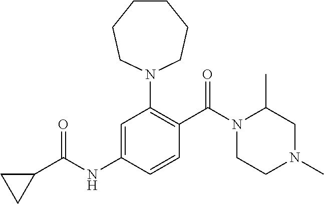 [SMILES: CC1CN(C)CCN1C(=O)c1ccc(NC(=O)C2CC2)cc1N1CCCCCC1]

### Example 37

The synthesis of the title compound was effected analogously to the synthesis of the compound of Example 35 (N-(3-(azepan-1-yl)-4-(piperidine-1-carbonyl)phenyl)cyclopropanecarboxamide) wherein 1,2-dimethyl-piperazine was used instead of piperidine. The title compound was obtained in a yield of 48% (42 mg).

1H NMR (400 MHz, DMSO-d6) δ 10.13 (s, 1H), 7.34 (d, 1H), 7.01 (dd, 1H), 6.92 (s, 1H), 3.70-3.62 (m, 1H), 3.55-3.46 (m, 1H), 3.26-3.08 (m, 6H), 2.45-2.12 (m, 6H), 1.81-1.36 (m, 11H), 0.85 (t, 3H), 0.80-0.76 (m, 4H); LC-MS: m/z 399.3 (MH+).

Preparation: N-(3-(azepan-1-yl)-4-(4-(trifluoromethyl)piperidine-1-carbonyl)phenyl) cyclopropanecarboxamide

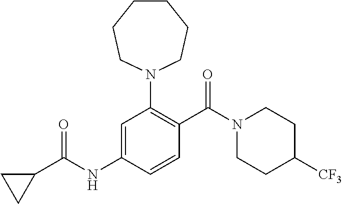 [SMILES: O=C(Nc1ccc(C(=O)N2CCC(C(F)(F)F)CC2)c(N2CCCCCC2)c1)C1CC1]

### Example 38

The synthesis of the title compound was effected analogously to the synthesis of the compound of Example 35 (N-(3-(azepan-1-yl )-4-(piperidine-1-carbonyl)phenyl)cyclopropanecarboxamide) wherein 4-(trifluoromethyl)piperidine hydrochloride was used instead of piperidine. The title compound was obtained in a yield of 54% (35 mg).

1H NMR (400 MHz, DMSO-d6) d ppm 10.19-10.06 (m, 1 H), 7.41-7.30 (m, 1 H), 6.98-6.88 (m, 2 H), 6.90 (s, 1 H), 4.69-4.54 (m, 1 H), 3.51-3.37 (m, 1 H), 3.25-3.12 (m, 4 H), 3.08-2.89 (m, 1 H), 2.76-2.53 (m, 2 H), 1.96-1.09 (m, 12 H), 0.79 (m, 4 H); LC-MS: m/z 438.3 (MH+).

Preparation: N-(3-(azepan-1-yl)-4-(1,2,3,4-tetrahydroisoquinoline-2-carbonyl)phenyl) cyclopropanecarboxamide

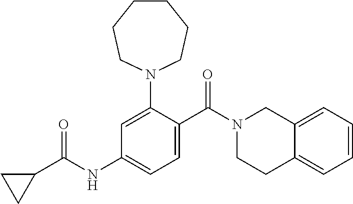 [SMILES: O=C(Nc1ccc(C(=O)N2CCc3ccccc3C2)c(N2CCCCCC2)c1)C1CC1]

### Example 21

The synthesis of the title compound was effected analogously to the synthesis of the compound of Example 35 (N-(3-(azepan-1-yl)-4-(piperidine-1-carbonyl)phenyl)cyclopropanecarboxamide) wherein 1,2,3,4-tetrahydroisoquinoline was used instead of piperidine. The title compound was obtained in a yield of 68% (42 mg).

1H NMR (400 MHz, DMSO-d6) d ppm 10.16 (s, 1 H), 7.37 (dd, 1 H), 7.28-6.92 (m, 6 H), 4.90-4.25 (m, 2 H), 4.05-3.93 (m, 1 H), 3.40 (d, 1 H), 3.25-3.01 (m, 4 H), 2.93-2.69 (m, 2 H), 1.82-1.24 (m, 9 H), 0.87-0.71 (m, 4 H); LC-MS: m/z 418.4 (MH+).

Preparation: N-(3-(azepan-1-yl)-4-(1,2,3,4-tetrahydropyrrolo[1,2-a]pyrazine-2-carbonyl)phenyl) cyclopropane carboxamide

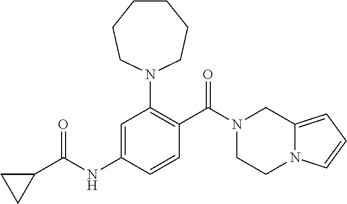 [SMILES: O=C(Nc1ccc(C(=O)N2CCn3cccc3C2)c(N2CCCCCC2)c1)C1CC1]

### Example 39

The synthesis of the title compound was effected analogously to the synthesis of the compound of Example 35 (N-(3-(azepan-1-yl)-4-(piperidine-1-carbonyl)phenyl)cyclopropanecarboxamide) wherein 1,2,3,4-tetrahydropyrrolo[1,2-a]pyrazine was used instead of piperidine. The title compound was obtained in a yield of 70% (42 mg).

1H NMR (400 MHz, DMSO-d6) d ppm 10.16 (s, 1 H), 7.38 (dd, 1 H), 7.08-6.94 (m, 2 H), 6.66 (d, 1 H), 6.07-5.94 (m, 1 H), 5.92-5.63 (m, 1 H), 4.96-4.22 (m, 2 H), 4.07-3.76 (m, 3 H), 3.51 (s, 1 H), 3.20 (t, 4 H), 1.81-1.26 (m, 9 H), 0.88-0.69 (m, 4 H); LC-MS: m/z 407.4 (MH+).

Preparation: N-[3-(azepan-1-yl)-4-{octahydropyrrolo[1,2-a]pyrazine-2-carbonyl}phenyl] Cyclopropanecarboxamide

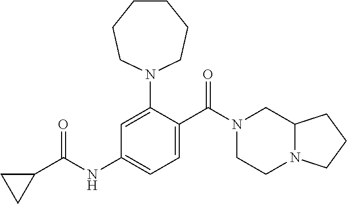 [SMILES: O=C(Nc1ccc(C(=O)N2CCN3CCCC3C2)c(N2CCCCCC2)c1)C1CC1]

### Example 40

The synthesis of the title compound was effected analogously to the synthesis of the compound of Example 35 (N-(3-(azepan-1-yl)-4-(piperidine-1-carbonyl)phenyl)cyclopropanecarboxamide) wherein 1,2,3,4,6,7,8,8a-octahydropyrrolo[1,2-a]pyrazine was used instead of piperidine. The title compound was obtained in a yield of 65% (65 mg).

¹ NMR (400 MHz, Chloroform-d) δ 7.56-7.38 (m, 2H), 7.17-7.00 (m, 1H), 6.71 (ddd, 1H), 4.83 (ddd, 1H), 3.71-2.46 (m, 9H), 2.30-1.23 (m, 18H), 1.17-1.05 (m, 2H); LC-MS: m/z 411.5 (MH+).

Preparation: N-(3-(azepan-1-yl)-4-(3-(trifluoromethyl)-5,6,7,8-tetrahydro-[1,2,4]triazolo[4,3-a]pyrazine-7-carbonyl)phenypcyclopropanecarboxamide

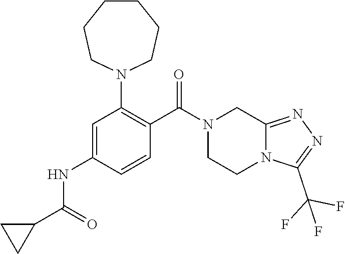 [SMILES: O=C(Nc1ccc(C(=O)N2CCn3c(nnc3C(F)(F)F)C2)c(N2CCCCCC2)c1)C1CC1]

### Example 41

The synthesis of the title compound was effected analogously to the synthesis of the compound of Example 35 (N-(3-(azepan-1-yl)-4-(piperidine-1-carbonyl)phenyl)cyclopropanecarboxamide) wherein 5,6,7,8-Tetrahydro-3-(trifluoromethyl)[1,2,4]triazolo[4,3-a]pyrazine was used instead of piperidine. The title compound was obtained in a yield of 72% (51 mg).

1H NMR (400 MHz, DMSO-d6) δ ppm 10.30-10.12 (m, 1 H), 7.53-7.36 (m, 1 H), 7.18-6.97 (m, 2 H), 5.31-4.46 (m, 2 H), 4.35-3.61 (m, 4 H), 3.26-3.02 (m, 4 H), 1.77 (quin, 1 H), 1.69-1.30 (m, 8 H), 0.87 -0.73 (m, 4 H); LC-MS: m/z 477.4 (MH+).

Preparation: 2-(azepan-1-yl)-4-cyclopropaneamido-N-[(pyridin-2-yl)methyl]benzamide

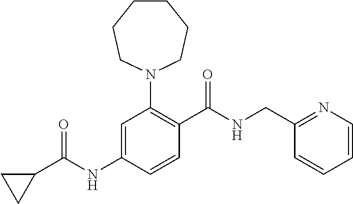 [SMILES: O=C(NCc1ccccn1)c1ccc(NC(=O)C2CC2)cc1N1CCCCCC1]

### Example 42

The synthesis of the title compound was effected analogously to the synthesis of the compound of Example 35 (N-(3-(azepan-1-yl )-4-(piperidine-1-carbonyl)phenyl)cyclopropanecarboxamide) wherein 2-pyridinylmethanamine was used instead of piperidine. The title compound was obtained in a yield of 43% (50 mg).

¹ NMR (400 MHz, DMSO-d₆) δ 10.31 (s, 1H), 10.11 (t, 1H), 8.53 (ddd, 1H), 7.78 (td, 1H), 7.68 (d, 1H), 7.61 (d, 1H), 7.39 (d, 1H), 7.35-7.26 (m, 1H), 7.23 (dd, 1H), 4.60 (d, 2H), 3.16-3.00 (m, 4H), 1.84-1.76 (m, 1H), 1.69 (s, 4H), 1.53 (p, 4H), 0.81 (dt, 4H); LC-MS: m/z 393.4 (MH+).

Preparation: 2-(azepan-1-yl)-N-benzyl-4-cyclopropaneamidobenzamide

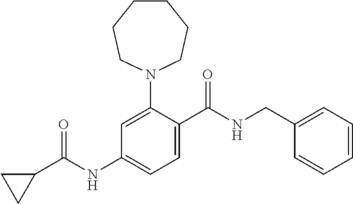 [SMILES: O=C(NCc1ccccc1)c1ccc(NC(=O)C2CC2)cc1N1CCCCCC1]

### Example 43

The synthesis of the title compound was effected analogously to the synthesis of the compound of Example 35 (N-(3-(azepan-1-yl)-4-(piperidine-1-carbonyl)phenyl)cyclopropanecarboxamide) wherein benzylamine was used instead of piperidine. The title compound was obtained in a yield of 69% (66 mg).

¹H NMR (400 MHz, Chloroform-d) δ 10.86 (s, 1H), 8.22 (d, 1H), 7.94 (s, 1H), 7.52 (s, 1H), 7.43-7.29 (m, 5H), 6.98 (dd, 1H), 4.67 (d, 2H), 3.11— 3.03 (m, 4H), 1.60 —1.56 (m, 4H), 1.54 —1.51 (m, 1H), 1.47 (p, 4H), 1.12 (dd, 2H), 0.96-0.85 (m, 2H); LC-MS: 392.4 (MH+).

Preparation: 2-(azepan-1-yl)-4-(cyclopropanecarbonylamino)-N-(pyridin-3-ylmethyl)benzamide

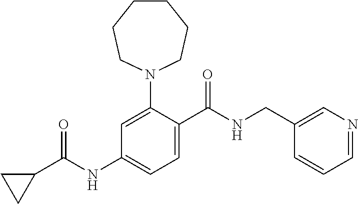 [SMILES: O=C(NCc1cccnc1)c1ccc(NC(=O)C2CC2)cc1N1CCCCCC1]

### Example 44

The synthesis of the title compound was effected analogously to the synthesis of the compound of Example 35 (N-(3-(azepan-1-yl)-4-(piperidine-1-carbonyl)phenyl)cyclopropanecarboxamide) wherein 3-pyridinylmethanamine was used instead of piperidine. The title compound was obtained in a yield of 72% (69 mg).

¹ NMR (400 MHz, Chloroform-d) δ 11.13 (s, 1H), 8.68-8.64 (m, 1H), 8.55 (dd, 1H), 8.21 (d, 1H), 7.97 (s, 1H), 7.76 (dd, 1H), 7.54 (d, 1H), 7.28 (s, 1H), 7.00 (dd, 1H), 4.69 (d, 2H), 3.24-2.96 (m, 4H), 1.59 (s, 9H), 1.24— 1.10 (m, 2H), 0.91 (dd, 2H); LC-MS: 393.4 (MH+).

Preparation: N-(3-(azepan-1-yl)-4-(morpholine-4-carbonyl)phenyl)cyclopropanecarboxamide

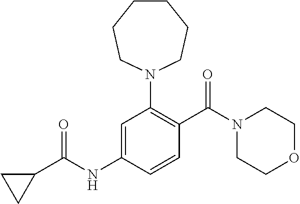 [SMILES: O=C(Nc1ccc(C(=O)N2CCOCC2)c(N2CCCCCC2)c1)C1CC1]

### Example 45

The synthesis of the title compound was effected analogously to the synthesis of the compound of Example 35 (N-(3-(azepan-1-yl)-4-(piperidine-1-carbonyl)phenyl)cyclopropanecarboxamide) wherein morpholine was used instead of piperidine. The title compound was obtained in a yield of 60% (66 mg).

¹ NMR (400 MHz, DMSO-d₆) δ 10.14 (s, 1H), 7.35 (d, 1H), 7.03 (dd, 1H), 6.95 (d, 1H), 3.76-3.40 (m, 6H), 3.26-3.07 (m, 6H), 1.81-1.44 (m, 9H), 0.81-0.74 (m, 4H); LC-MS: m/z 372.4 (MH+).

Preparation: N-(3-(azepan-1-yl)-4-(4-(pyridin-2-yl)piperazine-1-carbonyl)phenyl) Cyclopropanecarboxamide

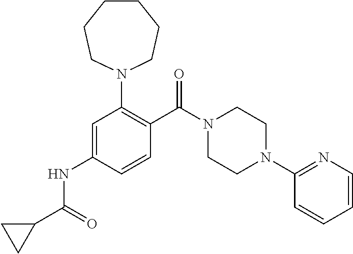 [SMILES: O=C(Nc1ccc(C(=O)N2CCN(c3ccccn3)CC2)c(N2CCCCCC2)c1)C1CC1]

### Example 46

The synthesis of the title compound was effected analogously to the synthesis of the compound of Example 35 (N-(3-(azepan-1-yl )-4-(piperidine-1-carbonyl)phenyl)cyclopropanecarboxamide) wherein 1-(2-pyridinyl)piperazine was used instead of piperidine. The title compound was obtained in a yield of 60% (77 mg).

¹ NMR (400 MHz, DMSO-d₆) δ 10.16 (s, 1H), 8.12 (ddd, 1H), 7.55 (ddd, 1H), 7.37 (d, 1H), 7.08-6.96 (m, 2H), 6.87-6.81 (m, 1H), 6.67 (ddd, 1H), 3.84-3.47 (m, 5H), 3.39-3.13 (m, 6H), 1.81-1.39 (m, 10H), 0.82-0.75 (m, 4H); LC-MS: m/z 448.4 (MH+)

Preparation: N-(3-(azepan-1-yl)-4-(4-propylpiperidine-1-carbonyl)phenyl)cyclopropanecarboxamide

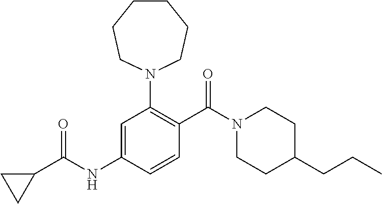 [SMILES: CCCC1CCN(C(=O)c2ccc(NC(=O)C3CC3)cc2N2CCCCCC2)CC1]

### Example 47

The synthesis of the title compound was effected analogously to the synthesis of the compound of Example 35 (N-(3-(azepan-1-yl)-4-(piperidine-1-carbonyl)phenyl)cyclopropanecarboxamide) wherein 3-ethyl-N-methylhexan-1-amine was used instead of piperidine. The title compound was obtained in a yield of 45% (55 mg).

1H NMR (400 MHz, DMSO-d6) δ ppm 10.11 (s, 1 H), 7.31 (dd, 1 H), 7.03-6.97 (m, 1 H), 6.95-6.81 (m, 1 H), 3.27-3.09 (m, 4 H), 4.61-2.54 (m, 4 H), 1.79-1.39 (m, 12 H), 1.36-1.23 (m, 2 H), 1.22-0.82 (m, 4 H), 0.90-0.82 (m, 3 H), 0.81-0.72 (m, 4 H); LC-MS: m/z 412.5 (MH+).

Preparation: N-(3-(azepan-1-yl)-4-(4-ethoxypiperidine-1-carbonyl)phenyl)cyclopropanecarboxamide

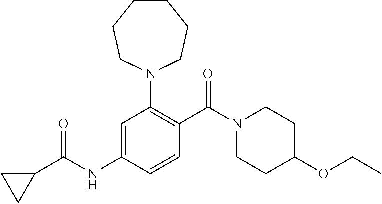 [SMILES: CCOC1CCN(C(=O)c2ccc(NC(=O)C3CC3)cc2N2CCCCCC2)CC1]

### Example 48

The synthesis of the title compound was effected analogously to the synthesis of the compound of Example 35 (N-(3-(azepan-1-yl)-4-(piperidine-1-carbonyl)phenyl)cyclopropanecarboxamide) wherein 4-ethoxypiperidin-1-ium chloride was used instead of piperidine. The title compound was obtained in a yield of 61% (75 mg).

1H NMR (400 MHz, DMSO-d6) δ ppm 10.12 (s, 1 H), 7.32 (d, 1 H), 7.04-6.96 (m, 1 H), 6.91 (dd, 1 H), 4.17-3.39 (m, 5 H), 3.31-2.87 (m, 6 H), 1.80-1.7 (m, 1 H), 2.11-1.16 (m, 12 H), 1.17-1.02 (m, 3 H), 0.83-0.72 (m, 4 H); LC-MS: m/z 414.4 (MH+).

Preparation: N-(3-(azepan-1-yl)-4-(4-methylpiperazine-1-carbonyl)phenyl)cyclopropanecarboxamide

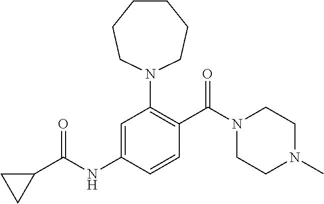 [SMILES: CN1CCN(C(=O)c2ccc(NC(=O)C3CC3)cc2N2CCCCCC2)CC1]

### Example 49

The synthesis of the title compound was effected analogously to the synthesis of the compound of Example 35 (N-(3-(azepan-1-yl )-4-(piperidine-1-carbonyl)phenyl)cyclopropanecarboxamide) wherein 1-methylpiperazine was used instead of piperidine. The title compound was obtained in a yield of 76% (86 mg).

1H NMR (400 MHz, DMSO-d6) δ 10.13 (s, 1 H), 7.33 (d, 1 H), 7.01 (dd, 1 H), 6.91 (d, 1 H), 3.88-2.96 (m, 8 H), 2.42-2.07 (m, 7 H), 1.83-1.42 (m, 9 H), 0.85-0.72 (m, 4 H); LC-MS: m/z 385.4 (MH+).

Preparation: N-(3-(azepan-1-yl)-4-(4-benzylpiperazine-1-carbonyl)phenyl)cyclopropanecarboxamide

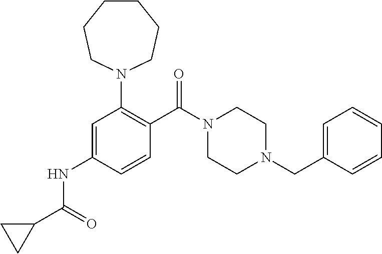 [SMILES: O=C(Nc1ccc(C(=O)N2CCN(Cc3ccccc3)CC2)c(N2CCCCCC2)c1)C1CC1]

### Example 50

The synthesis of the title compound was effected analogously to the synthesis of the compound of Example 35 (N-(3-(azepan-1-yl)-4-(piperidine-1-carbonyl)phenyl)cyclopropanecarboxamide) wherein 1-benzylpiperazine was used instead of piperidine. The title compound was obtained in a yield of 23% (31 mg). 1H NMR (500 MHz, DMSO-d6) δ ppm 10.13 (s, 1 H), 7.37-7.27 (m, 5 H), 7.27-7.22 (m, 1 H), 7.00 (dd, 1 H), 6.90 (d, 1 H), 3.49 (d, 2 H), 3.69-3.08 (m, 4 H), 3.26-3.08 (m, 4 H), 2.46-2.02 (m, 4 H), 1.79 -1.71 (m, 1 H), 1.71-1.44 (m, 8 H), 0.86-0.70 (m, 4 H); LC-MS: m/z 461.4 (MH+).

Preparation: N-(3-(azepan-1-yl)-4-(4-(pyridin-3-yl)piperazine-1-carbonyl)phenyl)

Cyclopropanecarboxamide

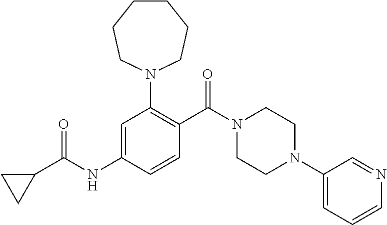 [SMILES: O=C(Nc1ccc(C(=O)N2CCN(c3cccnc3)CC2)c(N2CCCCCC2)c1)C1CC1]

### Example 51

The synthesis of the title compound was effected analogously to the synthesis of the compound of Example 35 (N-(3-(azepan-1-yl )-4-(piperidine-1-carbonyl)phenyl)cyclopropanecarboxamide) wherein 1-pyridin-3-yl-piperazine was used instead of piperidine. The title compound was obtained in a yield of 20% (26 mg).

1H NMR (500 MHz, DMSO-d6) δ ppm 10.16 (s, 1 H), 8.31 (d, 1 H), 8.02 (dd, 1 H), 7.37 (d, 1 H), 7.33 (ddd, 1 H), 7.22 (dd, 1 H), 7.08-7.01 (m, 1 H), 7.00-6.94 (m, 1 H), 3.24-3.19 (m, 4 H), 3.89-3.00 (m, 8 H), 1.81 - 1.73 (m, 1 H), 1.73-1.60 (m, 4 H), 1.60-1.43 (m, 4 H), 0.83-0.75 (m, 4 H); LC-MS: m/z 448.4 (MH+).

Preparation: N-(3-(azepan-1-yl)-4-(4-phenylpiperazine-1-carbonyl)phenyl)cyclopropanecarboxamide

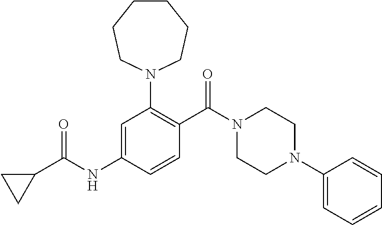 [SMILES: O=C(Nc1ccc(C(=O)N2CCN(c3ccccc3)CC2)c(N2CCCCCC2)c1)C1CC1]

### Example 52

The synthesis of the title compound was effected analogously to the synthesis of the compound of Example 35 (N-(3-(azepan-1-yl)-4-(piperidine-1-carbonyl)phenyl)cyclopropanecarboxamide) wherein 1-phenylpiperazine was used instead of piperidine. The title compound was obtained in a yield of 28% (21 mg).

1H NMR (400 MHz, DMSO-d6) δ ppm 10.15 (s, 1 H), 7.36 (d, 1 H), 7.28-7.16 (m, 2 H), 7.08-7.01 (m, 1 H), 6.97 (d, 1 H), 6.94 (d, 2 H), 6.80 (t, 1 H), 3.91-3.26 (m, 4 H), 3.26-2.91 (m, 8 H), 1.84-1.72 (m, 1 H), 1.67 (br s, 4 H), 1.60-1.39 (m, 4 H), 0.91-0.64 (m, 4 H); LC-MS: m/z 447.4 (MH+).

Preparation: N-[3-(azepan-1-yl)-4-(4-cyclopentylpiperazine-1-carbonyl)phenyl]cyclopropanecarboxamide

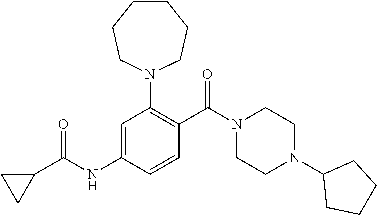 [SMILES: O=C(Nc1ccc(C(=O)N2CCN(C3CCCC3)CC2)c(N2CCCCCC2)c1)C1CC1]

### Example 53

The synthesis of the title compound was effected analogously to the synthesis of the compound of Example 35 (N-(3-(azepan-1-yl)-4-(piperidine-1-carbonyl)phenyl)cyclopropanecarboxamide) wherein 1-cyclopentylpiperazine was used instead of piperidine. The title compound was obtained in a yield of 68% (73 mg).

¹ NMR (400 MHz, Chloroform-d) δ 7.46 (s, 1H), 7.40 (s, 1H), 7.11 (d, 1H), 6.72 (dd, 1H), 4.11 (d, 1H), 3.53 (t, 1H), 3.31 (tt, 6H), 2.77-2.11 (m, 6H), 1.96-1.61 (m, 12H), 1.52-1.34 (m, 4H), 1.11 (q, 2H), 0.88 (dt, 2H); LC-MS: m/z 439.5 (MH+).

Preparation: N-[3-(azepan-1-yl)-4-[2-(1-benzyltetrazol-5-yl)piperidine-1-carbonyl]phenyl]cyclopropanecarboxamide

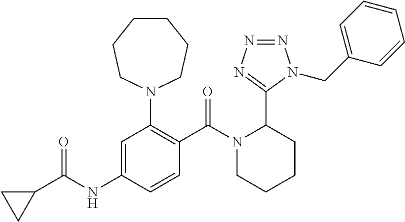 [SMILES: O=C(Nc1ccc(C(=O)N2CCCCC2c2nnnn2Cc2ccccc2)c(N2CCCCCC2)c1)C1CC1]

### Example 54

The synthesis of the title compound was effected analogously to the synthesis of the compound of Example 35 (N-(3-(azepan-1-yl)-4-(piperidine-1-carbonyl)phenyl)cyclopropanecarboxamide) wherein 2-(1-benzyltetrazol-5-yl)piperidine was used instead of piperidine. The title compound was obtained in a yield of 60% (64 mg).

¹ NMR (400 MHz, Chloroform-d) δ 7.51-7.34 (m, 6H), 7.13-6.96 (m, 1H), 5.81-5.69 (m, 2H), 5.19 -4.59 (m, 1H), 3.76-3.53 (m, OH), 3.45-3.17 (m, 4H), 3.11-2.77 (m, 1H), 2.55-2.36 (m, 1H), 2.07 -1.91 (m, 0H), 1.90- 1.25 (m, 19H), 1.10 (dq, OH), 0.85 (tt, 2H); LC-MS: m/z 528.3 (MH+).

Preparation: N-[3-(azepan-1-yl )-4-(3,4-dimethylpiperazine-1-carbonyl)phenyl]cyclopropanecarboxamide

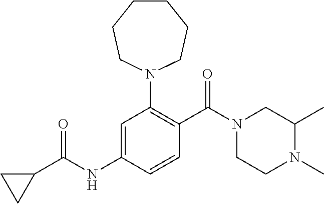 [SMILES: CC1CN(C(=O)c2ccc(NC(=O)C3CC3)cc2N2CCCCCC2)CCN1C]

### Example 55

The synthesis of the title compound was effected analogously to the synthesis of the compound of Example 35 (N-(3-(azepan-1-yl )-4-(piperidine-1-carbonyl)phenyl)cyclopropanecarboxamide) wherein 1,2-dimethyl-piperazine was used instead of piperidine. The title compound was obtained in a yield of 48% (42 mg).

¹ NMR (400 MHz, DMSO-d₆) δ 10.13 (d, 1H), 7.35 (dd, 1H), 7.05-6.86 (m, 2H), 4.42-4.12 (m, 1H), 3.25-2.90 (m, 6H), 2.88-2.57 (m, 2H), 2.17 (t, 3H), 2.10-1.37 (m, 11H), 0.94 (ddd, 3H), 0.82-0.75 (m, 4H); LC-MS: m/z 399.3 (MH+).

Preparation: 2-(azepan-1-yl)-4-(cyclopropanecarbonylamino)-N-[[4-(4-methylpiperazin-1-yl)phenyl]methyl]benzamide

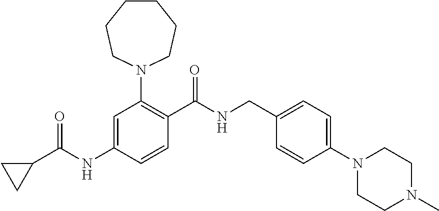 [SMILES: CN1CCN(c2ccc(CNC(=O)c3ccc(NC(=O)C4CC4)cc3N3CCCCCC3)cc2)CC1]

### Example 56

The synthesis of the title compound was effected analogously to the synthesis of the compound ofExample 35 (N-(3-(azepan-1-yl)-4-(piperidine-1-carbonyl)phenyl)cyclopropanecarboxamide) wherein [4-(4-methylpiperazin-1-yl)phenyl]methanamine was used instead of piperidine. The title compound was obtained in a yield of 59% (64 mg). 1H NMR (400 MHz, DMSO-d6) δ 10.27 (s, 1 H), 9.83-9.70 (m, 1 H), 7.66-7.50 (m, 2 H), 7.18 (d, 3 H), 6.89 (d, 2 H), 4.35 (d, 2 H), 3.15-2.97 (m, 8 H), 2.47-2.38 (m, 4 H), 2.21 (s, 3 H), 1.81-1.70 (m, 1 H), 1.63-1.50 (m, 4 H), 1.44 (d, 4 H), 0.85-0.72 (m, 4 H); LC-MS: 490.4 (MH+).

Preparation: N-[3-(azepan-1-yl )-4-(4-pyridin-4-ylpiperazine-1-carbonyl)phenyl]cyclopropanecarboxamide

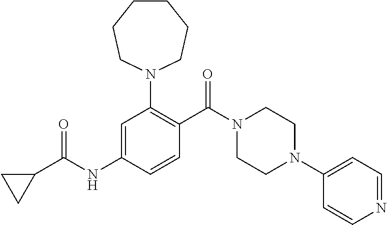 [SMILES: O=C(Nc1ccc(C(=O)N2CCN(c3ccncc3)CC2)c(N2CCCCCC2)c1)C1CC1]

### Example 57

The synthesis of the title compound was effected analogously to the synthesis of the compound of Example 35 (N-(3-(azepan-1-yl )-4-(piperidine-1-carbonyl)phenyl)cyclopropanecarboxamide) wherein 1-pyridin-4-ylpiperazine was used instead of piperidine. The title compound was obtained in a yield of 24% (32mg).

¹ NMR (400 MHz, DMSO-d₆) δ 10.16 (s, 1H), 8.27-7.99 (m, 2H), 7.37 (d, 1H), 7.07-6.96 (m, 2H), 6.85-6.79 (m, 2H), 3.81 (dd, 1H), 3.62 (dd, 1H), 3.46 (dd, 1H), 3.41— 3.34 (m, 3H), 3.22 (td, 6H), 1.81-1.73 (m, 1H), 1.67 (s, 4H), 1.59-1.45 (m, 4H), 0.87-0.70 (m, 4H); LC-MS: m/z 448.4 (MH+)

Preparation: N-[4-(3-phenylthiomorpholine-4-carbonyl)-3-pyrrolidin-1-ylphenyl]cyclopropanecarboxamide

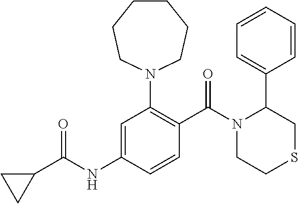 [SMILES: O=C(Nc1ccc(C(=O)N2CCSCC2c2ccccc2)c(N2CCCCCC2)c1)C1CC1]

### Example 58

To a solution of 4-(cyclopropanecarbonylamino)-2-pyrrolidin-1-ylbenzoic acid (229.52 mg, 0.840 mmol) and HATU: [dimethylamino(3-triazolo[4,5-b]pyridinyloxy)methylidene]-dimethylammonium;hexafluorophosphate (477.2 mg, 1.26 mmol) in MeCN (2.4 mL), sequentially N,N-Diisopropylethylamine (324.4 mg, 2.51 mmol) and 3-phenylthiomorpholine (150.0 mg, 0.840 mmol) were added. The reaction mixture was heated to 70° C. and stirred ON. After completion of the reaction the mixture was cooled down to RT and then it was poured in a s. s. of NaHCO₃ (30 mL) and AcOEt (45 mL).

Phases were separated and the organic layer was washed with a saturated solution of NH₄CI, dried over Na₂SO₄ and concentrate to dryness under vacuum. The residue was purified by FC on silica gel (eluting from cHex/AcOEt 8:2 to cHex/AcOEt 2:8) affording the product of formula N-[4-(3-phenylthiomorpholine-4-carbonyl)-3-pyrrolidin-1-yl phenyl]cyclopropanecarboxamide.
Yield: 180 mg

¹ NMR (400 MHz, DMSO-d₆) δ 10.18-10.01 (m, 1H), 7.58-7.19 (m, 5H), 7.19-6.76 (m, 3H), 6.17-4.52 (m, 1H), 3.83-3.65 (m, 1H), 3.29-3.21 (m, 4H), 3.16-2.58 (m, 4H), 2.46-2.29 (m, 1H), 2.03-1.62 (m, 5H), 0.89-0.61 (m, 4H); LC-MS: m/z 436.17 (MH+).

Preparation: N-[4-(4,4-difluoropiperidine-1-carbonyl)-3-pyrrolidin-1-ylphenyl]cyclopropanecarboxamide

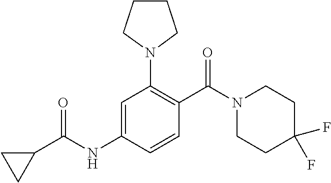 [SMILES: O=C(Nc1ccc(C(=O)N2CCC(F)(F)CC2)c(N2CCCC2)c1)C1CC1]

### Example 59

A solution of 4-(cyclopropanecarbonylamino)-2-pyrrolidin-1-ylbenzoic acid (100.0 mg, 6 0.360 mmol), N,N-Diisopropylethylamine (0.19 mL, 1.09 mmol) and HATU: ([dimethylamino(3-triazolo[4,5-b]pyridinyloxy)methylidene]-dimethylammonium hexafluorophosphate (207.91 mg, 0.550 mmol)) in DMF (1.823 mL) was stirred for 15 minutes at RT. Then 4, 4-difluropiperidine (53.43 mg, 0.440 mmol) was added and the reaction mixture was stirred at 80° C. for 2 hours. After this time a saturated solution of NaHCO₃ was added ant the reaction was extracted with AcOEt 3 times. The organic fractions were collected, washed with Brine, dried over Na₂SO₄, filtered and the solvent was evaporated under reduced pressure. The residue was purified by FC on RP using acidic conditions (eluting from CH₃CN/H₂O 5:95 +0.1% of formic acid to CH₃CN/H₂O 95:5 +0.1% of formic acid) affording the product of formula N-[4-(4,4-difluoropiperidine-1-carbonyl)-3-pyrrolidin-1-ylphenyl]cyclopropanecarboxamide.
Yield: 58 mg

1H NMR (400 MHz, DMSO-d6) δ 10.11 (s, 1H), 7.07 (d, 1H), 7.00 (d, 1H), 6.94 (dd, 1H), 4.00-3.84 (m, 1H), 3.29 (s, 1H), 3.55-3.39 (m, 2H), 3.23-2.98 (m, 4H), 2.14-1.82 (m, 8H), 1.77 (tt, 1H), 0.85-0.72 (m, 4H); LC-MS: m/z 378.17 (MH+).

Preparation: N-[4-(1,1-dioxo-1,4-thiazinane-4-carbonyl)-3-pyrrolidin-1-yl phenyl] Cyclopropanecarboxamide

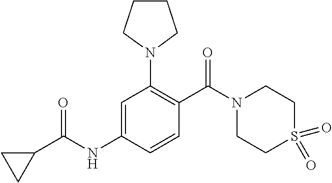 [SMILES: O=C(Nc1ccc(C(=O)N2CCS(=O)(=O)CC2)c(N2CCCC2)c1)C1CC1]

### Example 60

The synthesis of the title compound was effected analogously to the synthesis of the compound of Example 59 (N-[4-(4,4-difluoropiperidine-1-carbonyl)-3-pyrrolidin-1-yl phenyl]cyclopropanecarboxamide) wherein 1,4-thiazinane 1,1-dioxide hydrochloride was used instead of 4, 4-difluropiperidine. The title compound was obtained in a yield of 46% (66 mg).

¹ NMR (400 MHz, DMSO-d6) δ 10.12 (s, 1H), 7.11 (d, 1H), 7.07 (d, 1H), 6.95 (dd, 1H), 4.38 (d, 1H), 3.78 (d, 1H), 3.62 (dt, 2H), 3.25-2.98 (m, 8H), 1.93-1.81 (m, 4H), 1.82-1.71 (m, 1H), 0.88-0.71 (m, 4H); LC-MS: m/z 390.17 (MH+).

Preparation: N-[3-pyrrolidin-1-yl-4-[4-(trifluoromethyl)piperidine-1-carbonyl]phenyl]cyclopropanecarboxamide

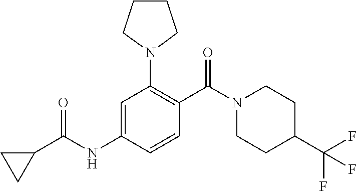 [SMILES: O=C(Nc1ccc(C(=O)N2CCC(C(F)(F)F)CC2)c(N2CCCC2)c1)C1CC1]

### Example 61

The synthesis of the title compound was effected analogously to the synthesis of the compound of Example 59 (N-[4-(4,4-difluoropiperidine-1-carbonyl)-3-pyrrolidin-1-ylphenyl]cyclopropanecarboxamide) wherein 4-(trifluoromethyl)piperidine;hydrochloride was used instead of 4, 4-difluropiperidine. The title compound was obtained in a yield of 70% (52 mg).

1H NMR (400 MHz, DMSO-d6) δ 10.10 (d, 1H), 7.08 (d, 1H), 7.01-6.83(m, 2H), 4.61 (t, 1H), 3.60 (t, 1H), 3.22-3.09 (m, 3H), 2.98 (dd, 2H), 2.72 (q, 1H), 2.60 (s, 1H), 1.87 (d, 5H), 1.81-1.68 (m, 2H), 1.47-1.21 (m, 2H), 0.87-0.71 (m, 4H); LC-MS: m/z 410.20 (MH+).

Preparation: N-[4-(4-methyl-2-phenylpiperazine-1-carbonyl)-3-pyrrolidin-1-ylphenyl]Cyclopropanecarboxamide

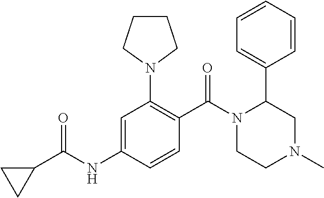 [SMILES: CN1CCN(C(=O)c2ccc(NC(=O)C3CC3)cc2N2CCCC2)C(c2ccccc2)C1]

### Example 62a, 62b, 62c

The synthesis of the title compound was effected analogously to the synthesis of the compound of Example 59 (N-[4-(4,4-difluoropiperidine-1-carbonyl)-3-pyrrolidin-1-ylphenyl]cyclopropanecarboxamide) wherein 1-methyl-3-phenylpiperazine was used instead of 4, 4-difluropiperidine. The title compound was obtained in a yield of 49% (38.7 mg) as a racemic mixture.

1H NMR (400 MHz, DMSO-d6) δ ppm 1H NMR (400 MHz, DMSO-d6) δ 10.16-10.06 (m, 1 H), 7.78-7.16 (m, 5 H), 7.16-6.75 (m, 3 H), 5.85-4.63 (m, 1 H), 4.51-3.11 (m, 4 H), 4.51-3.11 (m, 4 H), 3.08 -2.57 (m, 4 H), 2.28-2.16 (m, 3 H), 2.40-1.95 (m, 2 H), 1.79-1.71 (m, 1 H), 1.94-1.56 (m, 4 H), 0.83-0.69 (m, 4 H); LC-MS: m/z 433.27 (MH+).

The racemic mixture (Example 62c) was then separated into single enantiomers by chiral semi-preparative H PLC

| | Column | Chiralpak IC (25 × 2.0 cm), 5 μ | |
|---|---|---|---|
| | Mobile phase | n-Hexane/Ethanol 75/25% v/v | |
| | Flow rate (ml/min) | 17 ml/min | |
| | DAD detection | 220 nm | |
| Preparative chiral | Loop | 300 μL | |
| chromatography | Total amount | 35 mg | |
| protocol: | Injection | 5.3 mg (each injection) | |
| 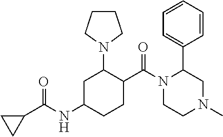 [SMILES: CN1CCN(C(=O)C2CCC(NC(=O)C3CC3)CC2N2CCCC2)C(c2ccccc2)C1] | Enantiomer 1 | Rt 10.83 min | 100% ee |
| 2R or 2S enantiomer | LC-MS: m/z 433.27 (MH+) | | |
| | (2R or 2S) n-[4-(4-methyl-2-phenylpiperazine-1-carbonyl)-3-pyrrolidin-1- | | |
| | ylphenyl] Cyclopropanecarboxamide | | |
| | Example 62a | | |
| 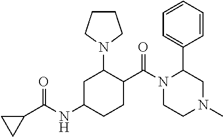 [SMILES: CN1CCN(C(=O)C2CCC(NC(=O)C3CC3)CC2N2CCCC2)C(c2ccccc2)C1] | Enantiomer 2 | Rt 13.23 min | 100% ee |

| | |
|---|---|
| 2R or 2S enantiomer | LC-MS: m/z 433.27 (MH+) |
| | (2R or 2S) n-[4-(4-methyl-2-phenylpiperazine-1-carbonyl)-3-pyrrolidin-1- |
| | ylphenyl] Cyclopropanecarboxamide |
| | Example 62b |

Preparation: 2R or 2S N-[4-(4-methyl-2-phenylpiperazine-1-carbonyl)-3-pyrrolidin-1-ylphenyl]cyclopropanecarboxamide hydrochloride

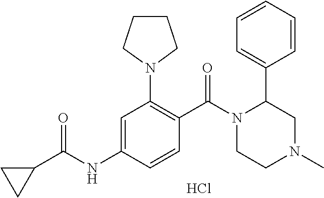 [SMILES: CN1CCN(C(=O)c2ccc(NC(=O)C3CC3)cc2N2CCCC2)C(c2ccccc2)C1 ; Cl]

### Example 62d

(2R or 2S) N-[4-(4-methyl-2-phenylpiperazine-1-carbonyl)-3-pyrrolidin-1-ylphenyl]Cyclopropanecarboxamide (Enantiomer 1, Example 62a, 10 mg) was dissolved in MeOH (1 mL) and treated with 1 eq of HCI in dioxane to afford, after evaporation, the product of formula 2R o 2S N-[4-(4-methyl-2-phenylpiperazine-1-carbonyl)-3-pyrrolidin-1-ylphenyl]cyclopropanecarboxamide hydrochloride.
Yield: 10 mg
LC-MS: m/z 433.32 (MH+)
   Preparation: N-[3-(3-methylpyrrolidin-1-yl )-4-[4-(trifluoromethyl)piperidine-1-carbonyl]phenyl]cyclopropanecarboxamide

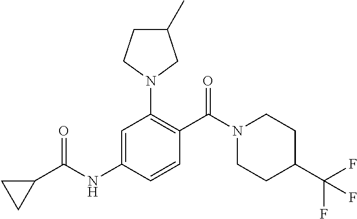 [SMILES: CC1CCN(c2cc(NC(=O)C3CC3)ccc2C(=O)N2CCC(C(F)(F)F)CC2)C1]

### Example 63a, 63b, 63c

The synthesis of the title compound was effected analogously to the synthesis of the compound of Example 59 (N-[4-(4,4-difluoropiperidine-1-carbonyl)-3-pyrrolidin-1-yl phenyl]cyclopropanecarboxamide) wherein 4-(trifluoromethyl)piperidine hydrochloride and 4-(cyclopropanecarbonylamino)-2-(3-methylpyrrolidin-1-yl )benzoic acid were used instead of 4, 4-difluropiperidine and 4-(cyclopropanecarbonylamino)-2-pyrrolidin-1-yl benzoic acid respectively. The title compound was obtained in a yield of 67% (98 mg) as a racemic mixture.

¹ NMR (400 MHz, DMSO-d₆) δ 10.14-10.05 (m, 1H), 7.11 (d, 1H), 6.98-6.84 (m, 2H), 4.78-3.40 (m, 1H), 3.27-2.53 (m, 6H), 2.37-1.66 (m, 5H), 1.60-1.15 (m, 5H), 1.10-1.01 (m, 3H), 0.86-0.73 (m, 4H); LC-MS: m/z 424.16 (MH+).

The racemic mixture (Example 63c) was then separated into single enantiomers by chiral semi-preparative H PLC

| | Column | Chiralpak AD-H (25 × 0.46 cm), 5 u | |
|---|---|---|---|
| | Mobile phase | n-Hexane/(Ethanol + 0.1% formic acid) | |
| | | 85/15% v/v | |
| | Flow rate (ml/min) | 17 ml/min | |
| | DAD detection | 220 nm | |
| Preparative chiral | Loop | 400 μL | |
| chromatography | Total amount | 95 mg | |
| protocol: | Injection | 7.6 mg (each injection) | |
| 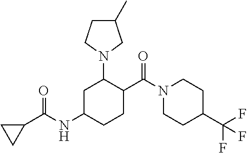 [SMILES: CC1CCN(C2CC(NC(=O)C3CC3)CCC2C(=O)N2CCC(C(F)(F)F)CC2)C1] | Enantiomer 1 | Rt 9.13 min | 100% ee |
| 3R or 3S enantiomer | LC-MS: m/z 424.16 (MH+) | | |
| | (N-[3-(3R or 3S-methylpyrrolidin-l-yl)-4-[4-(trifluoromethyl)piperidine-1- | | |
| | carbonyl]phenyl]cyclopropanecarboxamide | | |
| | Example 63a | | |
| 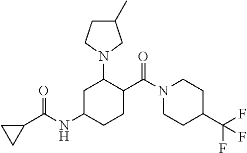 [SMILES: CC1CCN(C2CC(NC(=O)C3CC3)CCC2C(=O)N2CCC(C(F)(F)F)CC2)C1] | Enantiomer 2 | Rt 11.95 min | 100% ee |

| | |
|---|---|
| 3R or 3S enantiomer | LC-MS: m/z 424.16 (MH+) |
| | ((N-[3-(3R or 3S-methylpyrrolidin-1-yl)-4-[4-(trifluoromethyl)piperidine-1- |
| | carbonyl]phenyl]cyclopropanecarboxamide |
| | Example 63b |

Preparation: N-[4-(4-methyl-2-phenylpiperazine-1-carbonyl)-3-(3-methylpyrrolidin-1-ypphenyl]cyclopropanecarboxamide

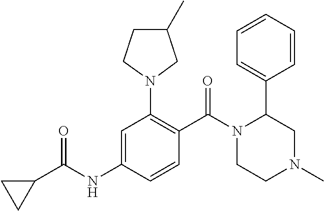 [SMILES: CC1CCN(c2cc(NC(=O)C3CC3)ccc2C(=O)N2CCN(C)CC2c2ccccc2)C1]

### Example 64a, 64b, 64c, 64d

A stirred solution of 4-(cyclopropanecarbonylamino)-2-(3-methylpyrrolidin-1-yl )benzoic acid (200.0 mg, 0.690 mmol), N,N-Diisopropylethylamine (0.36 mL, 2.08 mmol) and HATU ([dimethylamino(3-triazolo[4,5-b]pyridinyloxy)methylidene]dimethylammonium hexafluorophosphate (395.61 mg, 1.04 mmol)) in DMF (7.291 mL) was stirred for 15 minutes at RT. Then 1-methyl-3-phenylpiperazine (146.71 mg, 0.830 mmol) was added and the reaction mixture was stirred at RT ON. The day after a saturated solution of NaHCO₃ was added to the reaction mixture and the aqueous phase was extracted with AcOEt 3 times. The organic portions were collected, washed with brine, dried over Na₂SO₄ and the solvent was evaporated under reduced pressure. The residue was purified by FC on NH column (eluting from 100% of cHex to 100% of AcOEt) affording the product of formula N-[4-(4-methyl-2-phenylpiperazine-1-carbonyl)-3-(3-methylpyrrolidin-1-yl)phenyl]cyclopropanecarboxamide as a mixture of diastereoisomers.
Yield: 150 mg
LC-MS: m/z 447.23 (MH+)

The mixture of diasteroisomers was then separated into single enantiomers by chiral semi-preparative H PLC

| | Column | Chiralpak IC (25 × 2.0 cm), 5 μ | |
|---|---|---|---|
| | Mobile phase | n-Hexane/Ethanol 80/20% v/v | |
| | Flow rate (ml/min) | 17 | ml/min |
| | DAD detection | 220 | nm |
| Preparative chiral | Loop | 300 | μL |
| chromatography | Total amount | 150 | mg |
| protocol: | Injection | 7.6 | mg (each injection) |

Diasteroisomer 1

| | |
|---|---|
| 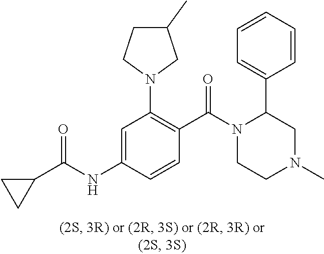 [SMILES: CC1CCN(c2cc(NC(=O)C3CC3)ccc2C(=O)N2CCN(C)CC2c2ccccc2)C1] | Enantiomer 1, Yield: 25 mg Rt 12.217 min 100% ee LC-MS: m/z 447.23 (MH+) (2S, 3R) or (2R, 3S) or (2R, 3R) or (2S, 3S) N-[4-(4-methyl-2-phenylpiperazine- 1-carbonyl)-3-(3-methylpyrrolidin-1-yl)phenyl] cyclopropanecarboxamide Example 64a |
| 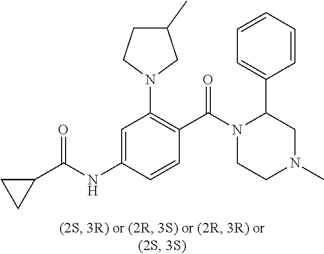 [SMILES: CC1CCN(c2cc(NC(=O)C3CC3)ccc2C(=O)N2CCN(C)CC2c2ccccc2)C1] | Enantiomer 2, Yield: 18 mg Rt 16.826 min 100% ee LC-MS: m/z 447.25 (MH+) (2S, 3R) or (2R, 3S) or (2R, 3R) or (2S, 3S) N-[4-(4-methyl-2- phenylpiperazine-1-carbonyl)-3-(3-methylpyrrolidin-1-yl)phenyl] cyclopropanecarboxamide Example 64b |

1H NMR (500 MHz, DMSO-d6 ) δ ppm 10.18-10.01 (m, 1 H), 7.76-7.35 (m, 1 H), 7.21-7.02 (m, 1 H), 6.98-6.89
(m, 1 H), 6.78-7.75 (m, 5 H), 4.87-5.53 (m, 1 H), 4.47-2.57 (m, 8 H), 2.27-2.11 (m, 3 H), 2.39-2.00 (m, 1 H),
1.81-1.68 (m, 1 H), 2.41-1.18 (m, 4 H), 0.83-0.71 (m, 4 H), 1.14-0.70 (m, 3 H),
Diasteroisomer 2

| | |
|---|---|
| 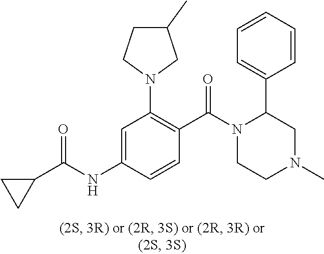 [SMILES: CC1CCN(c2cc(NC(=O)C3CC3)ccc2C(=O)N2CCN(C)CC2c2ccccc2)C1] | Enantiomer 1, Yield: 23 mg Rt 15.410 min 100% ee LC-MS: m/z 447.23 (MH+) (2S, 3R) or (2R, 3S) or (2R, 3R) or (2S, 3S) N-[4-(4-methyl-2-phenylpiperazine- 1-carbonyl)-3-(3-methylpyrrolidin-1-yl)phenyl] cyclopropanecarboxamide Example 64c |
| 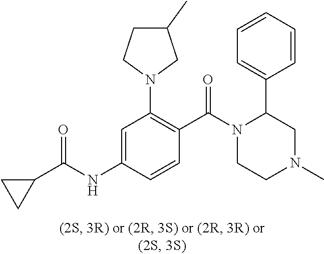 [SMILES: CC1CCN(c2cc(NC(=O)C3CC3)ccc2C(=O)N2CCN(C)CC2c2ccccc2)C1] | Enantiomer 2, Yield: 26 mg Rt 21.150 min 100% ee LC-MS: m/z 447.25 (MH+) (2S, 3R) or (2R, 3S) or (2R, 3R) or (2S, 3S) N-[4-(4-methyl-2- phenylpiperazine-1-carbonyl)-3-(3-methylpyrrolidin-1-yl)phenyl] cyclopropanecarboxamide Example 64d |

1H NMR (500 MHz, DMSO-d6 ) δ ppm 10.30-9.91 (m, 1 H), 7.19-7.00 (m, 1 H), 6.98-6.63 (m, 1 H), 7.97-6.56
(m, 6 H), 5.87-4.56 (m, 1 H), 4.54-2.52 (m, 8 H), 2.36-2.19 (m, 1 H), 2.27-2.16 (m, 3 H), 1.80-1.72 (m, 1 H),
2.02-1.18 (m, 2 H), 2.41-1.17 (m, 2 H), 1.12-0.82 (m, 3 H), 0.81-0.69 (m, 4 H).

Preparation: N-[3-(6-azaspiro[3.4]octan-6-yl)-4-(1,1-dioxo-1,4-thiazinane-4-carbonyl)phenyl]cyclopropanecarboxamide

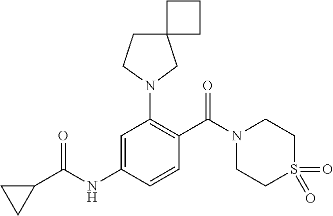 [SMILES: O=C(Nc1ccc(C(=O)N2CCS(=O)(=O)CC2)c(N2CCC3(CCC3)C2)c1)C1CC1]

### Example 65

2-(6-azaspiro[3.4]octan-6-yl)-4-(cyclopropanecarbonylamino)benzoic acid (112.0 mg, 0.360 mmol), HATU: [dimethylamino(3-triazolo[4,5-b]pyridinyloxy)methylidene]dimethylammonium;hexafluorophosphate (162.55 mg, 0.430 mmol) and N,N-Diisopropylethylamine (0.25 mL, 1.43 mmol) were mixed in DMF (3 mL) and stirred for 5 min, then thiomorpholine 1,1-dioxide (48.16 mg, 0.360 mmol) was added. The reaction mixture was stirred at RT overnight. The day after Water was added and the mixture was extracted with AcOEt 3 times. Combined organics were washed with Brine and dried over a phase separator, then concentrated under vacuum. The residue was purified by FC on on RP using basic conditions (eluting from 100% of Water +0.1% of NH₄OH to Water +0.2% NH₄OH/CH₃CN 6:4) affording the product of formula (N-[3-(6-azaspiro[3.4]octan-6-yl)-4-(1,1-dioxo-1,4-thiazinane-4- carbonyl)phenyl]Cyclopropanecarboxamide.
Yield: 26.8 mg 1H NMR (600 MHz, DMSO-d6) δ 10.12 (s, 1 H), 7.16-7.03 (m, 2 H), 6.90 (dd, 1 H), 4.33-4.45 (m, 1 H), 3.71-3.83 (m, 1 H), 3.70-3.60 (m, 1 H), 3.59-3.48 (m, 1 H), 3.36-2.97 (m, 8 H), 2.04-1.80 (m, 8 H), 1.79-1.72 (m, 1 H), 0.90-0.65 (m, 4 H); LC-MS: m/z 432.2 (MH+).
   Preparation: N-[3-(6-azaspiro[3.4]octan-6-yl)-4-(4-methyl-2-phenylpiperazine-1-carbonyl)phenyl]cyclopropanecarboxamide

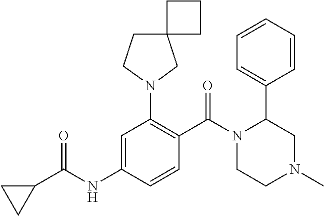 [SMILES: CN1CCN(C(=O)c2ccc(NC(=O)C3CC3)cc2N2CCC3(CCC3)C2)C(c2ccccc2)C1]

### Example 66a, 66b, 66c

The synthesis of the title compound was effected analogously to the synthesis of the compound of Example 65 (N-[3-(6-azaspiro[3.4]octan-6-yl)-4-(1,1-dioxo-1,4-thiazinane-4-carbonyl)phenyl]cyclopropanecarboxamide) wherein 1-methyl-3-phenylpiperazine was used instead of thiomorpholine 1,1-dioxide. The title compound was obtained in a yield of 20% (30 mg) as a racemic mixture. 1H NMR (400 MHz, DMSO-d6) δ 10.18-10.00 (m, 1 H), 7.16-6.99 (m, 1 H), 6.99-6.92 (m, 1 H), 6.9-6.84 (m, 1 H), 7.84-6.66 (m, 5 H), 5.87-4.60 (m, 1 H), 3.28-3.20 (m, 2 H), 3.06-2.61 (m, 2 H), 4.48-2.29 (m, 6 H), 2.27-2.10 (m, 3 H), 1.80-1.69 (m, 1 H), 2.09-1.53 (m, 8 H), 0.84-0.67(m, 4 H); LC-MS: m/z 473.2 (MH+).

The racemic mixture (Example 66c) was then separated into single enantiomers by chiral semi-preparative H PLC

| | Column | Chiralpak IC (25 × 2.0 cm), 5 μ | |
|---|---|---|---|
| | Mobile phase | n-Hexane/Ethanol 80/20% v/v | |
| | Flow rate (ml/min) | 17 | ml/min |
| | DAD detection | 220 | nm |
| Preparative chiral | Loop | 750 | μl |
| chromatography | Total amount | 27 | mg |
| protocol: | Injection | 9 | mg (each injection) |

| | |
|---|---|
| 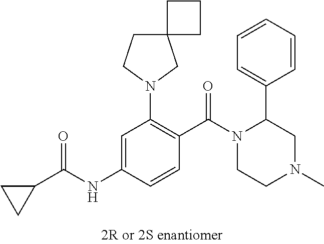 [SMILES: CN1CCN(C(=O)c2ccc(NC(=O)C3CC3)cc2N2CCC3(CCC3)C2)C(c2ccccc2)C1] | Enantiomer 1 Rt 15.16 min 100% ee LC-MS: m/z 473.2 (MH+) (N-[3-(3R or 3S-methylpyrrolidin-1-yl)-4-[4-(trifluoromethyl)piperidine-1- carbonyl]phenyl]cyclopropanecarboxamide Example 66a |
| 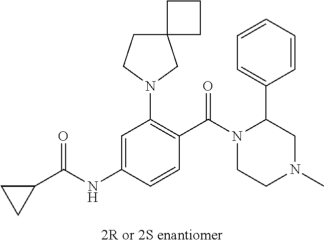 [SMILES: CN1CCN(C(=O)c2ccc(NC(=O)C3CC3)cc2N2CCC3(CCC3)C2)C(c2ccccc2)C1] | Enantiomer 2 Rt 20.45 min 100% ee LC-MS: m/z 473.2 (MH+) ((N-[3-(3R or 3S-methylpyrrolidin-1-yl)-4-[4-(trifluoromethyl)piperidine-1- carbonyl]phenyl]cyclopropanecarboxamide Example 66b |

Preparation: Syn N-[3-[-3,5-dimethylpiperidin-1-yl]-4-(1,1-dioxo-1,4-thiazinane-4-carbonyl)phenyl]Cyclopropanecarboxamide

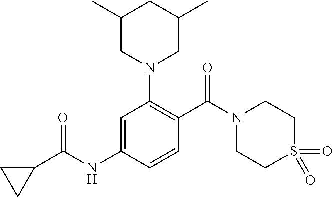 [SMILES: CC1CC(C)CN(c2cc(NC(=O)C3CC3)ccc2C(=O)N2CCS(=O)(=O)CC2)C1]

### Example 67

The synthesis of the title compound was effected analogously to the synthesis of the compound of Example 65 (N-[3-(6-azaspiro[3.4]octan-6-yl)-4-(1,1-dioxo-1,4-thiazinane-4-carbonyl)phenyl]Cyclopropanecarboxamide) wherein Syn 4-(cyclopropanecarbonylamino)-2-[(3,5-dimethylpiperidin-1-yl]benzoic acid was used instead of 2-(6-azaspiro[3.4]octan-6-yl)-4-(cyclopropanecarbonylamino)benzoic acid. The title compound was obtained in a yield of 20% (21 mg).

¹ NMR (400 MHz, DMSO-d6) δ ppm 10.27 (s, 1 H), 7.42 (d, 1 H), 7.26 (dd, 1 H), 7.15 (d, 1 H), 4.29-3.39 (m, 4 H), 3.30-3.00 (m, 2 H), -3.61-2.97 (m, 4 H), 2.38-1.88 (m, 2 H), 1.84-1.72 (m, 2 H), 1.71-1.48 (m, 2 H), 0.82-0.74 (m, 4 H), 0.99-0.71 (m, 6 H), 0.62 (q, 1 H); LC-MS: m/z 434.2 (MH+)

Preparation: Syn N-[3-[3,5-dimethylpiperidin-1-yl]-4-(4-methyl-2-phenylpiperazine-1-carbonyl)phenyl]cyclopropanecarboxamide

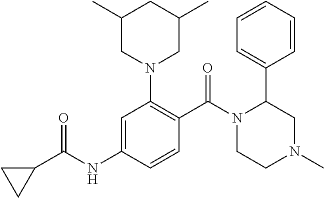 [SMILES: CC1CC(C)CN(c2cc(NC(=O)C3CC3)ccc2C(=O)N2CCN(C)CC2c2ccccc2)C1]

### Example 68a, 68b, 68c, 68d, 68e

The synthesis of the title compound was effected analogously to the synthesis of the compound of Example 66(N-[3-(6-azaspiro[3.4]octan-6-yl)-4-(4-methyl-2-phenylpiperazine-1-carbonyl)phenyl]cyclopropanecarboxamide) wherein Syn 4-(cyclopropanecarbonylamino)-2-[(3,5-dimethylpiperidin-1-yl]benzoic acid was used instead of 2-(6-azaspiro[3.4]octan-6-yl)-4-(cyclopropanecarbonylamino)benzoic acid. The title compound was obtained in a yield of 62% (92 mg) as a racemic mixture.

1H NMR (400 MHz, DMSO-d6) δ ppm 10.31-10.06 (m, 1 H), 7.81-6.94 (m, 8 H), 5.74 (br s, 1 H), 2.94 -2.22 (m, 4 H), 2.17 (s, 2 H), 1.82-1.76 (m, 1 H), 4.43-1.69 (m, 6 H), 2.01-1.53 (m, 3 H), 2.07-1.03 (m, 1 H), 0.82-0.74 (m, 4 H), 0.71-0.41 (m, 1 H), 0.99-0.38 (m, 6 H); LC-MS: m/z 475.3 (MH+). The racemic mixture (Example 68c) was then separated into single enantiomers by chiral semi-preparative H PLC

| | Column | Chiralpak IC (25 × 2.0 cm), 5 μ | |
|---|---|---|---|
| | Mobile phase | n-Hexane/Ethanol 80/20% v/v | |
| | Flow rate (ml/min) | 17 | ml/min |
| | DAD detection | 220 | nm |
| Preparative chiral | Loop | 400 | μL |
| chromatography | Total amount | 83 | mg |
| protocol: | Injection | 7.12 | mg (each injection) |

| | |
|---|---|
| 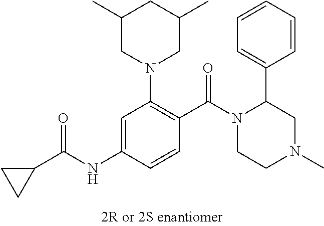 [SMILES: CC1CC(C)CN(c2cc(NC(=O)C3CC3)ccc2C(=O)N2CCN(C)CC2c2ccccc2)C1] | Enantiomer 1 Rt 7.87 min 100% ee LC-MS: m/z 475.3 (MH+) Syn 2R or 2S N-[3-[3,5-dimethylpiperidin-1-yl]-4-(4-methyl-2- phenylpiperazine-1-carbonyl)phenyl] cyclopropanecarboxamide Example 68a |
| 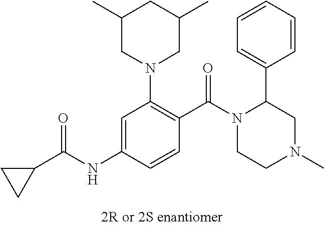 [SMILES: CC1CC(C)CN(c2cc(NC(=O)C3CC3)ccc2C(=O)N2CCN(C)CC2c2ccccc2)C1] | Enantiomer 2 Rt 9.97 min 100% ee LC-MS: m/z 475.3 (MH+) Syn 2R or 2S N-[3-[3,5-dimethylpiperidin-1-yl]-4-(4-methyl-2- phenylpiperazine-1-carbonyl)phenyl] cyclopropanecarboxamide Example 68b |

Preparation: Anti N-[3-[3,5-dimethylpiperidin-1-yl]-4-(4-methyl-2-phenylpiperazine-1-carbonyl)phenyl]cyclopropanecarboxamide

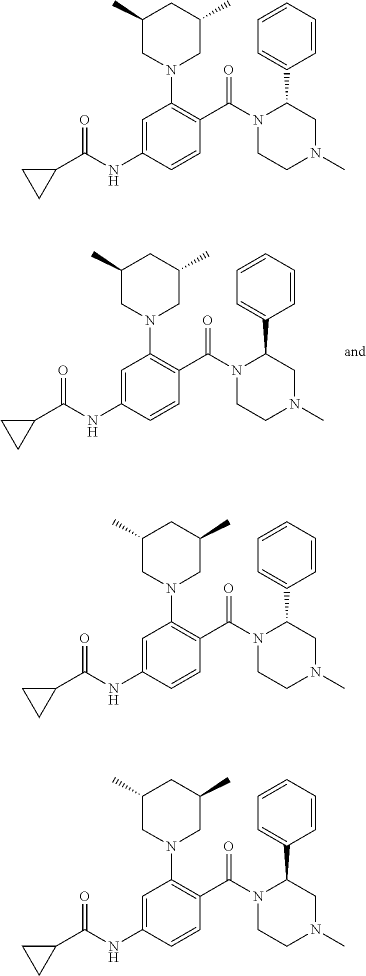 [SMILES: C[C@H]1C[C@H](C)CN(c2cc(NC(=O)C3CC3)ccc2C(=O)N2CCN(C)C[C@H]2c2ccccc2)C1 ; C[C@H]1C[C@H](C)CN(c2cc(NC(=O)C3CC3)ccc2C(=O)N2CCN(C)C[C@@H]2c2ccccc2)C1 ; C[C@@H]1C[C@@H](C)CN(c2cc(NC(=O)C3CC3)ccc2C(=O)N2CCN(C)C[C@H]2c2ccccc2)C1 ; C[C@@H]1C[C@@H](C)CN(c2cc(NC(=O)C3CC3)ccc2C(=O)N2CCN(C)C[C@@H]2c2ccccc2)C1]

To a solution of 4-(cyclopropanecarbonylamino)-2-(3,5-dimethylpiperidin-1-yl)benzoic acid (50.0 mg, 0.160 mmol) in DMF (1.58 mL) and N,N-Diisopropylethylamine (0.07 mL, 0.400 mmol), HATU: [dimethylamino(3-triazolo[4,5-b]pyridinyloxy)methylidene]-dimethylammonium hexafluorophosphate (60.09 mg, 0.160 mmol) was added and the mixture was stirred at RT for 30 min. After this time 1-methyl-3-phenylpiperazine (32.03 mg, 0.180 mmol) was added and the reaction was stirred at room temperature overnight. The day after H₂O was added and a precipitate formation was observed. The solid was filtered and washed with H₂O. The afforded solid was firstly purified by FC on RP using acidic (eluting from CH₃CN/H₂O 5:95 +0.1% of formic acid to CH₃CN/H₂O 6:4+0.1% of formic acid) and then further purified by Semi preparative MDAP Method:

| | | |
|---|---|---|
| | Column: | XSelect CSH Prep. C18 5 μm OBD 30 × 100 mm @ room T |
| | Solvents: | A = H₂O + 0.1% HCOOH B = Acetonitrile |
| | Method: | from 30% B to 36% B in 10 min |
| Semi preparative achiral | Injection loop: | 1 mL (each injection) |
| chromatography | Flow rate (ml/min): | 40 ml/min |
| protocol: | UV detection range: | 210 nm to 350 nm |

| | |
|---|---|
| 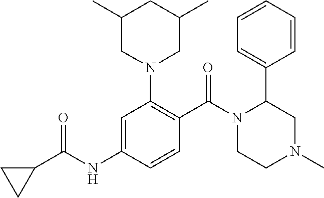 [SMILES: CC1CC(C)CN(c2cc(NC(=O)C3CC3)ccc2C(=O)N2CCN(C)CC2c2ccccc2)C1] | First eluted peak: Yield: 9.6% (7.2 mg) ¹H NMR (400 MHz, DMSO-d₆) δ 10.19 (d, 1H), 7.48 (d, 2H), 7.41-7.15 (m, 5H), 7.12-6.79 (m, 1H), 5.85-3.42 (m, 1H), 3.27-2.57 (m, 7H), 2.46-2.11 (m, 4H), 1.87 (d, 4H), 1.50-1.22 (m, 2H), 1.02 (dd, 5H), 0.77 (dd, 6H); LC-MS: m/z 475.12 (MH+). Example 68d |
| 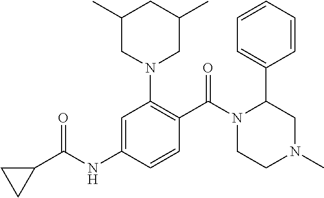 [SMILES: CC1CC(C)CN(c2cc(NC(=O)C3CC3)ccc2C(=O)N2CCN(C)CC2c2ccccc2)C1] | Second eluted peak Yield: 9.9% (7.4 mg) ¹H NMR (400 MHz, DMSO-d₆) δ 10.23 (d, 1H), 7.78-7.66 (m, 1H), 7.55-7.43 (m, 2H), 7.40-7.14 (m, 4H), 7.02 (dd, 1H), 5.84-5.65 (m, 1H), 4.55-3.39 (m, 1H), 3.20 (dd, 1H), 3.10-2.88 (m, 2H), 2.83-2.75 (m, 2H), 2.71-2.61 (m, 1H), 2.39- 2.11 (m, 4H), 2.08-1.93 (m, 2H), 1.86-1.62 (m, 2H), 1.45-1.13 (m, 2H), 0.98 (dd, 4H), 0.87-0.58 (m, 7H); LC-MS: m/z 475.12 (MH+). Example 68e |

Preparation: Syn 2R or 2S N-[3-[3,5-dimethylpiperidin-1-yl]-4-(4-methyl-2-phenylpiperazine-1-carbonyl)phenyl]cyclopropanecarboxamide hydrochloride

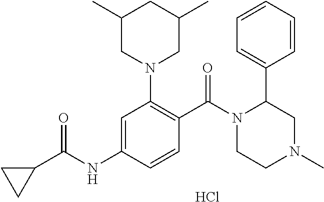 [SMILES: CC1CC(C)CN(c2cc(NC(=O)C3CC3)ccc2C(=O)N2CCN(C)CC2c2ccccc2)C1 ; Cl]

### Example 68f

Syn 2R or 2S N-[3-[3,5-dimethylpiperidin-1-yl]-4-(4-methyl-2-phenylpiperazine-1-carbonyl)phenyl]cyclopropanecarboxamide (Enantiomer 1, Example 68a , 14 mg) was dissolved in MeOH (1 mL) and treated with 1 eq of HCI in dioxane to afford, after evaporation, the product of formula Syn 2R or 2S N-[3-[3,5-dimethylpiperidin-1-yl]-4-(4-methyl-2-phenylpiperazine-1-carbonyl)phenyl]cyclopropanecarboxamide hydrochloride.
Yield: 10.1 mg
LC-MS: 475.33 (MH+)
   Preparation: N-[4-(2-phenylpiperazine-1-carbonyl)-3-pyrrolidin-1-yl phenyl]cyclopropanecarboxamide

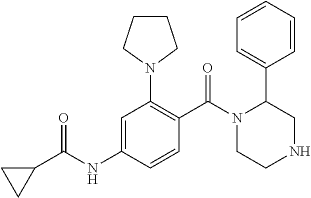 [SMILES: O=C(Nc1ccc(C(=O)N2CCNCC2c2ccccc2)c(N2CCCC2)c1)C1CC1]

To a solution of tert-butyl 4-[4-(cyclopropanecarbonylamino)-2-pyrrolidin-1-yl benzoyl]-3-phenylpiperazine-1-carboxylate (156.0 mg, 0.300 mmol) in DCM (3 mL) Trifluoroacetic acid (1.0 mL, 23.77 mmol) was added and the reaction was left standing at RT overnight. Solvent was removed under vacuum and the residue was purified by SCX washing with MeOH and eluting with 1N NH₃ in MeOH. Basic fractions were evaporated and the residue was purified by FC on NH column (eluting from 100% of DCM to DCM/MeOH 95:5) affording the product of formula N-[4-(2-phenylpiperazine-1-carbonyl)-3-pyrrolidin-1-ylphenyl]cyclopropanecarboxamide as a racemic mixture.
Yield: 85 mg.

1H NMR (500 MHz, DMSO-d6) δ ppm 10.18-9.98 (m, 1 H), 7.15-7.02 (m, 1 H), 6.99-6.70 (m, 1 H), 7.98-6.57 (m, 6 H), 5.71-4.57 (m, 1 H), 3.31-2.76 (m, 4 H), 2.84-2.54 (m, 2 H), 4.49-2.54 (m, 4 H), 1.79 -1.74 (m, 1 H), 2.01-1.54 (m, 4 H), 0.84-0.65 (m, 4 H); LC-MS: 419.2 (MH+).

Preparation: N-[4-[2-(2-fluorophenyl)piperazine-1-carbonyl]-3-pyrrolidin-1-ylphenyl]cyclopropanecarboxamide

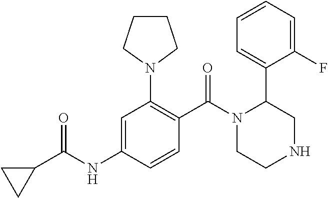 [SMILES: O=C(Nc1ccc(C(=O)N2CCNCC2c2ccccc2F)c(N2CCCC2)c1)C1CC1]

### Example 157

To a solution of tert-butyl tert-butyl 4-[4-(cyclopropanecarbonylamino)-2-pyrrolidin-1-ylbenzoyl]-3-(2-fluorophenyppiperazine-1-carboxylate (200.0 mg, 0.370 mmol) in DCM (3 mL) Trifluoroacetic acid (0.29 mL, 3.73 mmol) was added and the reaction was stirred at RT for 2 h. Solvent was removed under vacuum and the residue was purified by SCX washing with MeOH and eluting with 1N NH₃ in MeOH. Basic fractions were evaporated affording the product of formula N-[4-[2-(2-fluorophenyl)piperazine-1-carbonyl]-3pyrrolidin-1-ylphenyl]cyclopropanecarboxamide as a racemic mixture.
Yield: 95 mg
LC-MS: m/z 437.17 (MH+)
   Preparation: N-[4-[2-(4-fluorophenyl)piperazine-1-carbonyl]-3-pyrrolidin-1-ylphenyl]cyclopropanecarboxamide

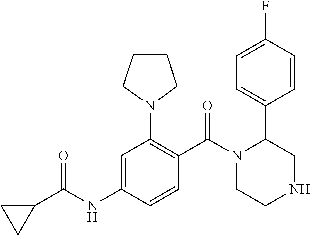 [SMILES: O=C(Nc1ccc(C(=O)N2CCNCC2c2ccc(F)cc2)c(N2CCCC2)c1)C1CC1]

The synthesis of the title compound was effected analogously to the synthesis of the compound of Example 157 (N-[4-[2-(2-fluorophenyl)piperazine-1-carbonyl]-3-pyrrolidin-1-ylphenyl]cyclopropanecarboxamide) wherein tert-butyl 4[4-(cyclopropanecarbonylamino)-2-pyrrolidin-1-ylbenzoyl]-3-(4-fluorophenyppiperazine-1-carboxylate was used instead of tert-butyl 4[4-(cyclopropanecarbonylamino)-2-pyrrolidin-1-ylbenzoyI]-3-(2-fluorophenyl)piperazine-1-carboxylate. The title compound was obtained in a yield of 95% (85 mg) as a racemic mixture.
LC-MS: m/z 437.18 (MH+).
   Preparation: N-[4[2-(3-fluorophenyl)piperazine-1-carbonyl]-3-pyrrolidin-1-ylphenyl]Cyclopropanecarboxamide

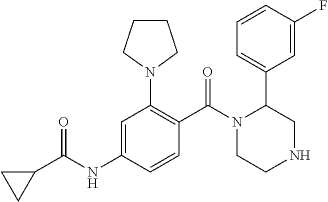 [SMILES: O=C(Nc1ccc(C(=O)N2CCNCC2c2cccc(F)c2)c(N2CCCC2)c1)C1CC1]

The synthesis of the title compound was effected analogously to the synthesis of the compound of Example 157 (N-[4[2-(2-fluorophenyl)piperazine-1-carbonyl]-3-pyrrolidin-1-ylphenyl]cyclopropanecarboxamide) wherein tert-butyl 4-[4-(cyclopropanecarbonylamino)-2-pyrrolidin-1-ylbenzoyl]-3-(3-fluorophenyl)piperazine-1-carboxylate was used instead of tert-butyl 4-[4-(cyclopropanecarbonylamino)-2-pyrrolidin-1-ylbenzoyI]-3-(2-fluorophenyl)piperazine-1-carboxylate. The title compound was obtained in a yield of 99% (75 mg) as a racemic mixture.
LC-MS: m/z 437.18 (MH+).
   Preparation: N-[3-(6-azaspiro[3.4]octan-6-yl)-4-(2-thiophen-2-ylpiperazine-1-carbonyl)phenyl]cyclopropanecarboxamide

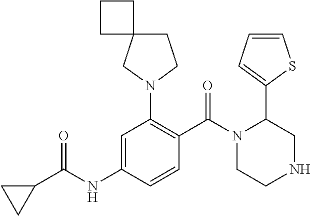 [SMILES: O=C(Nc1ccc(C(=O)N2CCNCC2c2cccs2)c(N2CCC3(CCC3)C2)c1)C1CC1]

The synthesis of the title compound was effected analogously to the synthesis of the compound of Example 157 (N-[4[2-(2-fluorophenyl)piperazine-1-carbonyl]-3-pyrrolidin-1-ylphenyl]cyclopropanecarboxamide) wherein tert-butyl 4-[2-(6-azaspiro[3.4]octan-6-yl)-4-(cyclopropanecarbonylamino)benzoyI]-3-thiophen-2-ylpiperazine-1-carboxylate was used instead of tert-butyl 4[4-(cyclopropanecarbonylamino)-2-pyrrolidin-1-ylbenzoyl]-3-(2-fluorophenyl)piperazine-1-carboxylate. The title compound was obtained in a yield of 60% (14 mg) as a racemic mixture.
LC-MS: m/z 465.18 (MH+).
   Preparation: N-[3-pyrrolidin-1-yl-4-(2-thiophen-2-ylpiperazine-1-carbonyl)phenyl]cyclopropanecarboxamide

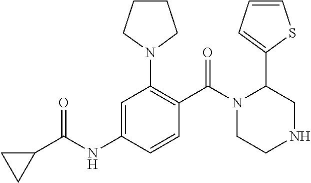 [SMILES: O=C(Nc1ccc(C(=O)N2CCNCC2c2cccs2)c(N2CCCC2)c1)C1CC1]

The synthesis of the title compound was effected analogously to the synthesis of the compound of Example 157 (N-[4[2-(2-fluorophenyppiperazine-1-carbonyl]-3-pyrrolidin-1-ylphenyl]cyclopropanecarboxamide) wherein tert-butyl 4-[4-(cyclopropanecarbonylamino)-2-pyrrolidin-1-ylbenzoyl]-3-thiophen-2-ylpiperazine-1-carboxylate was used instead of tert-butyl 4-[4-(cyclopropanecarbonylamino)-2-pyrrolidin-1-ylbenzoyI]-3-(2-fluorophenyl)piperazine-1-carboxylate. The title compound was obtained in a yield of 99% (170 mg) as a racemic mixture.
LC-MS: m/z 425.12 (MH+).
   Preparation: N-[4[2-(1-methylpyrazol-4-yppiperazine-1-carbonyl]-3-pyrrolidin-1-ylphenyl]cyclopropanecarboxamide

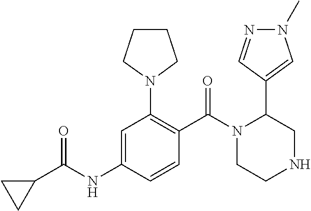 [SMILES: Cn1cc(C2CNCCN2C(=O)c2ccc(NC(=O)C3CC3)cc2N2CCCC2)cn1]

The synthesis of the title compound was effected analogously to the synthesis of the compound of Example 157 (N-[4-[2-(2-fluorophenyppiperazine-1-carbonyl]-3-pyrrolidin-1-ylphenyl]cyclopropanecarboxamide) wherein tert-butyl 4-[4-(cyclopropanecarbonylamino)-2-pyrrolidin-1-ylbenzoyl]-3-(1-methylpyrazol-4-yppiperazine-1-carboxylate was used instead of tert-butyl 4[4-(cyclopropanecarbonylamino)-2-pyrrolidin-1-ylbenzoyI]-3-(2-fluorophenyl)piperazine-1-carboxylate. The title compound was obtained in a yield of 88% (88 mg) as a racemic mixture.
LC-MS: m/z 425.12 (MH+).
   Preparation:N-[3-(azepan-1-yl)-4-[2-(thiophen-2-yl)piperazine-1-carbonyl]phenyl]cyclopropanecarboxamide

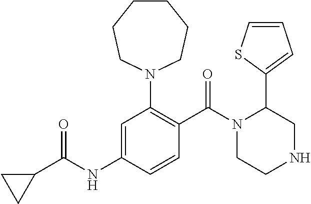 [SMILES: O=C(Nc1ccc(C(=O)N2CCNCC2c2cccs2)c(N2CCCCCC2)c1)C1CC1]

Tert-butyl 4-[2-(azepan-1-yl)-4-(cyclopropanecarbonylamino)benzoyl]-2-thiophen-2-ylpiperazine-1-carboxylate (145.0 mg, 0.260 mmol) was dissolved in DCM (4 mL) and Trifluoroacetic acid (0.2 mL, 2.62 mmol) was added. The mixture was stirred at RT overnight, then volatiles were removed under vacuum and the residue was charged on SCX washing with MeOH and eluting with 1N NH₃ in MeOH. Evaporation of basic fraction afforded the compound of formula (N-[3-(azepan-1-yl)-4-(2-thiophen-2-ylpiperazine-1-carbonyl)phenyl]cyclopropanecarboxamide) which was used in the next step without further purification
Yield: 94 mg
LC-MS: 453.2 (MH+).
   Preparation: N-[3-(azepan-1-yl)-4-(2-thiophen-3-ylpiperazine-1-carbonyl)phenyl]cyclopropanecarboxamide

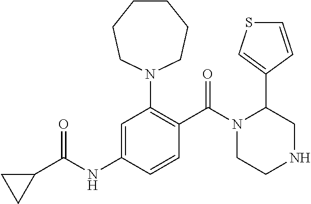 [SMILES: O=C(Nc1ccc(C(=O)N2CCNCC2c2ccsc2)c(N2CCCCCC2)c1)C1CC1]

The synthesis of the title compound was effected analogously to the synthesis of the compound of Example 35 (N-(3-(azepan-1-yl)-4-(piperidine-1-carbonyl)phenyl)cyclopropanecarboxamide) wherein tert-butyl 4-[2-(azepan-1-yl)-4-(cyclopropanecarbonylamino)benzoyl]-3-thiophen-3-ylpiperazine-1-carboxylate was used instead of Tert-butyl 4-[2-(azepan-1-yl)-4-(cyclopropanecarbonylamino)benzoyl]-2-thiophen-2-ylpiperazine-1-carboxylate. The title compound was obtained in a yield of 96% (51 mg).
LC-MS: m/z 453.2 (MH+)
   Preparation: N-[3-(azepan-1-yl)-4-[2-(1-methylpyrazol-4-yl)piperazine-1-carbonyl]phenyl]Cyclopropanecarboxamide

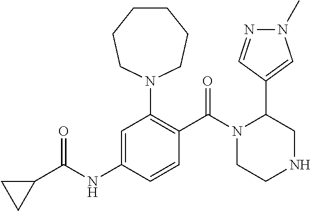 [SMILES: Cn1cc(C2CNCCN2C(=O)c2ccc(NC(=O)C3CC3)cc2N2CCCCCC2)cn1]

The synthesis of the title compound was effected analogously to the synthesis of the compound of Example 35 (N-(3-(azepan-1-yl)-4-(piperidine-1-carbonyl)phenyl)cyclopropanecarboxamide) wherein tert-butyl 4-[2-(azepan-1-yl)-4-(cyclopropanecarbonylamino)benzoyl]-3-(1-methylpyrazol-4-yl)piperazine-1-carboxylate was used instead of Tert-butyl 4-[2-(azepan-1-yl)-4-(cyclopropanecarbonylamino)benzoyl]-2-thiophen-2-ylpiperazine-1-carboxylate. The title compound was obtained in a yield of 92% (53 mg).
LC-MS: m/z 451.3 (MH+).
   Preparation: N-[3-(azepan-1-yl)-4-(2-pyridin-2-ylpiperazine-1-carbonyl)phenyl]cyclopropanecarboxamide

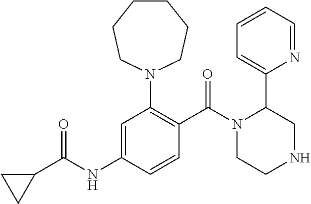 [SMILES: O=C(Nc1ccc(C(=O)N2CCNCC2c2ccccn2)c(N2CCCCCC2)c1)C1CC1]

The synthesis of the title compound was effected analogously to the synthesis of the compound of Example 35 (N-(3-(azepan-1-yl)-4-(piperidine-1-carbonyl)phenyl)cyclopropanecarboxamide) wherein tert-butyl 4-[2-(azepan-1-yl)-4-(cyclopropanecarbonylamino)benzoyl]-3-pyridin-2-ylpiperazine-1-carboxylate was used instead of Tert-butyl 4-[2-(azepan-1-yl)-4-(cyclopropanecarbonylamino)benzoyl]-2-thiophen-2-ylpiperazine-1-carboxylate. The title compound was obtained in a yield of 91% (60 mg).
LC-MS: m/z 448.3 (MH+).
   Preparation: N-[3-(azepan-1-yl)-4-[2-(trifluoromethyl)piperazine-1-carbonyl]phenyl]Cyclopropanecarboxamide

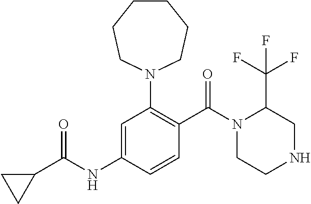 [SMILES: O=C(Nc1ccc(C(=O)N2CCNCC2C(F)(F)F)c(N2CCCCCC2)c1)C1CC1]

The synthesis of the title compound was effected analogously to the synthesis of the compound of Example 35 (N-(3-(azepan-1-yl)-4-(piperidine-1-carbonyl)phenyl)cyclopropanecarboxamide) wherein tert-butyl 4-[2-(azepan-1-yl)-4-(cyclopropanecarbonylamino)benzoyl]-3-(trifluoromethyppiperazine-1-carboxylate was used instead of Tert-butyl 4-[2-(azepan-1-yl)-4-(cyclopropanecarbonylamino)benzoyl]-2-thiophen-2-ylpiperazine-1-carboxylate. The title compound was obtained in a yield of 90% (33 mg). LC-MS: m/z 439.3 (MH+).

Preparation: N-[3-(azepan-1-yl)-4-(2-benzylpiperazine-1-carbonyl)phenyl]cyclopropanecarboxamide

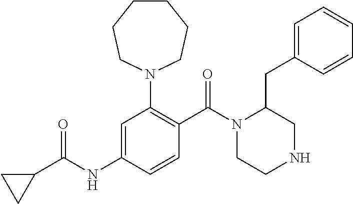 [SMILES: O=C(Nc1ccc(C(=O)N2CCNCC2Cc2ccccc2)c(N2CCCCCC2)c1)C1CC1]

The synthesis of the title compound was effected analogously to the synthesis of the compound of Example 35 (N-(3-(azepan-1-yl)-4-(piperidine-1-carbonyl)phenyl)cyclopropanecarboxamide) wherein tert-butyl 4-[2-(azepan-1-yl)-4-(cyclopropanecarbonylamino)benzoyl]-3-benzylpiperazine-1-carboxylate was used instead of Tert-butyl 4-[2-(azepan-1-yl)-4-(cyclopropanecarbonylamino)benzoyl]-2-thiophen-2-ylpiperazine-1-carboxylate. The title compound was obtained in a yield of 80% (65 mg). LC-MS: m/z 461.3 (MH+).

Preparation: N-[3-(azepan-1-yl)-4-[2-(hydroxymethyl)piperazine-1-carbonyl]phenyl]cyclopropanecarboxamide

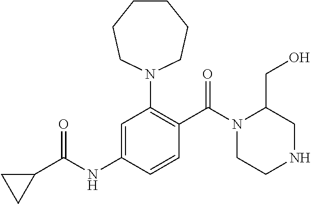 [SMILES: O=C(Nc1ccc(C(=O)N2CCNCC2CO)c(N2CCCCCC2)c1)C1CC1]

The synthesis of the title compound was effected analogously to the synthesis of the compound of Example 35 (N-(3-(azepan-1-yl)-4-(piperidine-1-carbonyl)phenyl)cyclopropanecarboxamide) wherein tert-butyl 4-[2-(azepan-1-yl)-4-(cyclopropanecarbonylamino)benzoyl]-3-(hydroxymethyppiperazine-1-carboxylate was used instead of Tert-butyl 4-[2-(azepan-1-yl)-4-(cyclopropanecarbonylamino)benzoyl]-2-thiophen-2-ylpiperazine-1-carboxylate. The title compound was obtained in a yield of 86% (72 mg). LC-MS: m/z 401.6 (MH+).

Preparation: N-[3-(azepan-1-yl)-4-(2-methylpiperazine-1-carbonyl)phenyl]cyclopropanecarboxamide

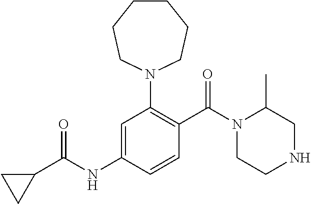 [SMILES: CC1CNCCN1C(=O)c1ccc(NC(=O)C2CC2)cc1N1CCCCCC1]

The synthesis of the title compound was effected analogously to the synthesis of the compound of Example 35 (N-(3-(azepan-1-yl)-4-(piperidine-1-carbonyl)phenyl)cyclopropanecarboxamide) wherein tert-butyl 4-[2-(azepan-1-yl)-4-(cyclopropanecarbonylamino)benzoyl]-3-methylpiperazine-1-carboxylate was used instead of Tert-butyl 4-[2-(azepan-1-yl)-4-(cyclopropanecarbonylamino)benzoyl]-2-thiophen-2-ylpiperazine-1-carboxylate. The title compound was obtained in a yield of 80% (70 mg). LC-MS: m/z 385.3 (MH+).

Preparation: N-[3-(azepan-1-yl)-4-(2-propan-2-ylpiperazine-1-carbonyl)phenyl]cyclopropanecarboxamide

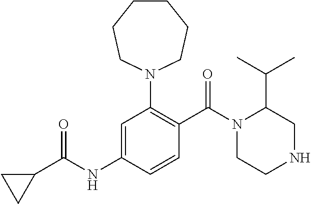 [SMILES: CC(C)C1CNCCN1C(=O)c1ccc(NC(=O)C2CC2)cc1N1CCCCCC1]

The synthesis of the title compound was effected analogously to the synthesis of the compound of Example 35 (N-(3-(azepan-1-yl)-4-(piperidine-1-carbonyl)phenyl)cyclopropanecarboxamide) wherein tert-butyl 4-[2-(azepan-1-yl)-4-(cyclopropanecarbonylamino)benzoyl]-3-propan-2-ylpiperazine-1-carboxylate was used instead of Tert-butyl 4-[2-(azepan-1-yl)-4-(cyclopropanecarbonylamino)benzoyl]-2-thiophen-2-ylpiperazine-1-carboxylate. The title compound was obtained in a yield of 83% (80 mg).
LC-MS: m/z 413.3 (MH+).
   Preparation: N-[3-(azepan-1-yl)-4-(2-phenylpiperazine-1-carbonyl)phenyl]cyclopropanecarboxamide

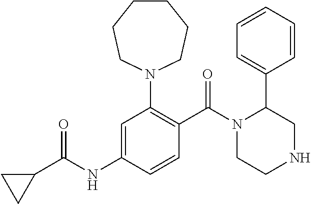 [SMILES: O=C(Nc1ccc(C(=O)N2CCNCC2c2ccccc2)c(N2CCCCCC2)c1)C1CC1]

The synthesis of the title compound was effected analogously to the synthesis of the compound of Example 35 (N-(3-(azepan-1-yl)-4-(piperidine-1-carbonyl)phenyl)cyclopropanecarboxamide) wherein tert-butyl 4-[2-(azepan-1-yl)-4-(cyclopropanecarbonylamino)benzoyl]-3-phenylpiperazine-1-carboxylate was used instead of Tert-butyl 4-[2-(azepan-1-yl)-4-(cyclopropanecarbonylamino)benzoyl]-2-thiophen-2-ylpiperazine-1-carboxylate. The title compound was obtained in a yield of 83% (80 mg).
LC-MS: m/z 447.3 (MH+).
   Preparation: N-[3-(azepan-1-yl)-4-(2-pyridin-2-ylpiperazine-1-carbonyl)phenyl]cyclopropanecarboxamide

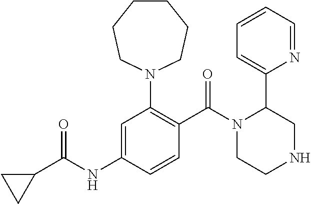 [SMILES: O=C(Nc1ccc(C(=O)N2CCNCC2c2ccccn2)c(N2CCCCCC2)c1)C1CC1]

The synthesis of the title compound was effected analogously to the synthesis of the compound of Example 35 (N-(3-(azepan-1-yl)-4-(piperidine-1-carbonyl)phenyl)cyclopropanecarboxamide) wherein tert-butyl 4-[2-(azepan-1-yl)-4-(cyclopropanecarbonylamino)benzoyl]-3-pyridin-2-ylpiperazine-1-carboxylate was used instead of Tert-butyl 4-[2-(azepan-1-yl)-4-(cyclopropanecarbonylamino)benzoyl]-2-thiophen-2-ylpiperazine-1-carboxylate. The title compound was obtained in a yield of 92% (60 mg). LC-MS: 448.3 (MH+).

Preparation: N-(3-(azepan-1-yl)-4-(4-methyl-2-(thiophen-2-yl)piperazine-1-carbonyl)phenyl) cyclopropane carboxamide

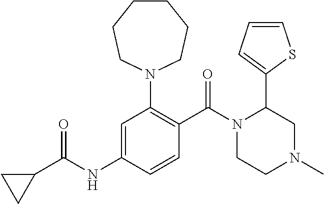 [SMILES: CN1CCN(C(=O)c2ccc(NC(=O)C3CC3)cc2N2CCCCCC2)C(c2cccs2)C1]

### Example 69a, 69b, 69c

To a mixture of N-[3-(azepan-1-yl)-4-(2-thiophen-2-ylpiperazine-1-carbonyl)phenyl]cyclopropanecarboxamide (94.0 mg, 0.210 mmol) and 37% formaldehyde in H₂O (0.15 mL, 2.08 mmol) in Methanol (5 mL), sodium triacetoxyborohydride (264.1 mg, 1.25 mmol) was added at 0° C. The reaction was warmed to RT and stirred ON. The day after the reaction was concentrated under vacuum, AcOEt and Water were added and the organic phase was separated. The aqueous phase was back-extracted twice with AcOEt, then the combined organic fractions were washed with Brine, dried over Na₂SO₄, filtered and concentrated under vacuum. The residue was purified by FC on silica gel (eluting from 100% of cHex to cHex/AcOEt 10:90) and it was further purified by FC on RP using basic conditions (eluting from 100% of Water +0.2% of NH₄OH to Water +0.2% NH4OH/CH₃CN 6:4) affording the product of formula N-[3-(azepan-1-yl)-4-(4-methyl-2-thiophen-2-ylpiperazine-1carbonyl)phenyl]cyclopropanecarboxamide.
Yield: 36 mg

1H NMR (400 MHz, DMSO-d6) δ 10.21-10.06 (m, 1 H), 7.47-7.24 (m, 2 H), 7.23 -6.55 (m, 4 H), 6.01 -4.27(m, 1 H), 2.27-2.19 (m, 3 H), 4.40-1.84 (m, 10 H), 1.81-1.67 (m, 1 H), 1.85-1.17 (m, 8 H), 0.86-0.69 (m, 4 H); LC-MS: 467.21 (MH+).

The racemic mixture (Example 69c) was then separated into single enantiomers by preparative HPLC

| | Column | Chiralpak AD-H (25 × 2.0 cm), 5 μ | |
|---|---|---|---|
| | Mobile phase | n-Hexane/(Ethanol + 0.1% TFA) 80/20 | |
| | % v/v | | |
| | Flow rate (ml/min) | 17 ml/min | |
| | DAD detection | 220 nm | |
| Preparative chiral | Loop | 800 μL | |
| chromatography | Total amount | 31 mg | |
| protocol: | Injection | 9.9 mg (each injection) | |
| 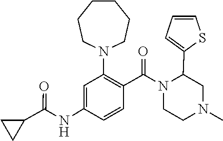 [SMILES: CN1CCN(C(=O)c2ccc(NC(=O)C3CC3)cc2N2CCCCCC2)C(c2cccs2)C1] | Enantiomer 1 | Rt 7.35 min | 95 ee |
| 2R or 2S enantiomer | LC-MS: m/z 467.21 (MH+) | | |
| | (2R or 2S) N-(3-(azepan-1-yl)-4-(4-methyl-2-(thiophen-2-yl)piperazine-1- | | |
| | carbonyl)phenyl) cyclopropane carboxamide | | |
| | Example 69a | | |
| 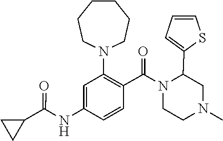 [SMILES: CN1CCN(C(=O)c2ccc(NC(=O)C3CC3)cc2N2CCCCCC2)C(c2cccs2)C1] | Enantiomer 2 | Rt 13.15 min | 99% ee |

| | |
|---|---|
| 2R or 2S enantiomer | LC-MS: m/z 467.21 (MH+) |
| | (2R or 2S) N-(3-(azepan-1-yl)-4-(4-methyl-2-(thiophen-2-yl)piperazine-1- |
| | carbonyl)phenyl) cyclopropane carboxamide |
| | Example 69b |

Preparation: N-(3-(azepan-1-yl)-4-(4-methyl-2-(thiophen-3-yl)piperazine-1-carbonyl)phenyl)cyclopropane carboxamide

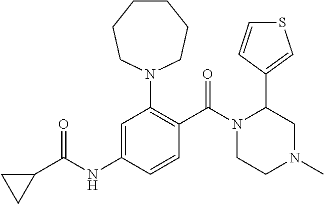 [SMILES: CN1CCN(C(=O)c2ccc(NC(=O)C3CC3)cc2N2CCCCCC2)C(c2ccsc2)C1]

### Example 70a, 70b, and 70c

The synthesis of the title compound was effected analogously to the synthesis of the compound of Example 69a-c (N-(3-(azepan-1-yl)-4-(4-methyl-2-(thiophen-2-yl)piperazine-1-carbonyl)phenyl) cyclopropane carboxamide) wherein N-[3-(azepan-1-yl)-4-(2-thiophen-3-ylpiperazine-1-carbonyl)phenyl]cyclopropanecarboxamide was used instead of N-[3-(azepan-1-yl)-4-(2-thiophen-2-ylpiperazine-1-carbonyl)phenyl]cyclopropanecarboxamide. The title compound was obtained in a yield of 32% (17 mg).

1H NMR (400 MHz, DMSO-d6) d pμm 10.06-10.27 (m, 1 H) 6.73-7.68 (m, 6 H) 4.29-5.77 (m, 1 H) 3.35- 3.41 (m, 1 H) 2.57-3.26 (m, 5 H) 2.09-2.38 (m, 4 H) 1.05-2.01 (m, 12 H) 0.66-0.86 (m, 4 H); LC-MS: m/z 467.3 (MH+).

The racemic mixture (Example 70c) was then separated into single enantiomers by preparative HPLC

| | Column | Chiralpak AD-H (25 × 2 cm), 5 μ | |
|---|---|---|---|
| | Mobile phase | n-Hexane/(Ethanol/Methanol 1:1 + 0.1% | |
| | | isopropylamine) 90/10% v/v | |
| | Flow rate (ml/min) | 17 | ml/min |
| | DAD detection | 220 | nm |
| Preparative chiral | Loop | 1500 | μL |
| chromatography | Total amount | 13 | mg |
| protocol: | Injection | 13 | mg (each injection) |

| | |
|---|---|
| 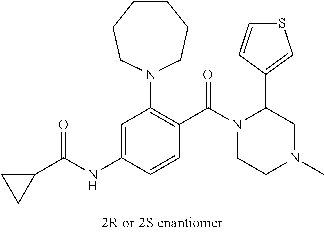 [SMILES: CN1CCN(C(=O)c2ccc(NC(=O)C3CC3)cc2N2CCCCCC2)C(c2ccsc2)C1] | Enantiomer 1 Rt 9.45 min 99% ee LC-MS: m/z 467.4 (MH+) (2R or 2S) N-(3-(azepan-1-yl)-4-(4-methyl-2-(thiophen-3-yl)piperazine-1- carbonyl)phenyl)cyclopropane carboxamide Example 70a |
| 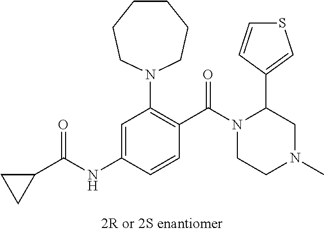 [SMILES: CN1CCN(C(=O)c2ccc(NC(=O)C3CC3)cc2N2CCCCCC2)C(c2ccsc2)C1] | Enantiomer 2 Rt 12.55 min 98% ee LC-MS: m/z 467.4 (MH+) (2R or 2S) N-(3-(azepan-1-yl)-4-(4-methyl-2-(thiophen-3-yl)piperazine-1- carbonyl)phenyl)cyclopropane carboxamide Example 70b |

Preparation: N-(3-(azepan-1-yl)-4-(4-methyl-2-(1-methyl-1H-pyrazol-3-yl)piperazine-1-carbonyl)phenyl)cyclopropane carboxamide

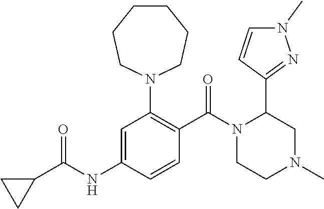 [SMILES: CN1CCN(C(=O)c2ccc(NC(=O)C3CC3)cc2N2CCCCCC2)C(c2ccn(C)n2)C1]

### Examples 71a and 71b

The synthesis of the title compound was effected analogously to the synthesis of the compound of Example 69a-c (N-(3-(azepan-1-yl)-4-(4-methyl-2-(thiophen-2-yl)piperazine-1-carbonyl)phenyl) cyclopropane carboxamide) wherein N-[3-(azepan-1-yl)-4-[2-(1-methylpyrazol-4-yl)piperazine-1-carbonyl]phenyl]cyclopropanecarboxamide was used instead of N-[3-(azepan-1-yl)-4-(2-thiophen-2-ylpiperazine-1-carbonyl)phenyl]cyclopropanecarboxamide. The title compound was obtained in a yield of 40% (21 mg).

1H NMR (500 MHz, DMSO-d6) δ ppm 10.27-9.96 (m, 1 H), 7.78 -7.49 (m, 1 H), 7.48-7.20 (m, 2 H), 7.05-6.75 (m, 2 H), 5.74-4.39(m, 1 H), 3.86-3.67 (m, 3 H), 2.22-2.16 (m, 3 H), 4.37-1.83 (m, 10 H), 1.81-1.27 (m, 9 H), 0.86-0.68 (m, 4 H); LC-MS: m/z 465.5 (MH+).

The racemic mixture was then separated into single enantiomers by preparative HPLC

| | Column | Chiralpak IC (25 × 0.46 cm), 5 u | |
|---|---|---|---|
| | Mobile phase | n-Hexane/(Ethanol + 0.1% | |
| | | isopropylamine) 60/40% v/v | |
| | Flow rate (ml/min) | 17 | ml/min |
| | DAD detection | 220 | nm |
| Preparative chiral | Loop | 650 | μL |
| chromatography | Total amount | 18.7 | mg |
| protocol: | Injection | 6 | mg (each injection) |

| | |
|---|---|
| 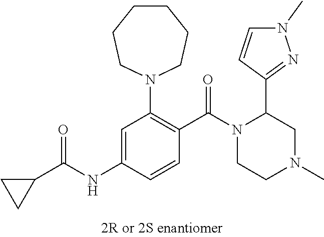 [SMILES: CN1CCN(C(=O)c2ccc(NC(=O)C3CC3)cc2N2CCCCCC2)C(c2ccn(C)n2)C1] | Enantiomer 1 Rt 10.36 min 99% ee LC-MS: m/z 465.5 (MH+) (2R or 2S)(N-(3-(azepan-1-yl)-4-(4-methyl-2-(1-methyl-1H-pyrazol-3- yl)piperazine-1-carbonyl)phenyl)cyclopropane carboxamide Example 71a |
| 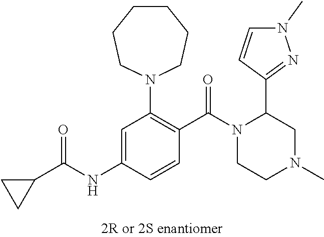 [SMILES: CN1CCN(C(=O)c2ccc(NC(=O)C3CC3)cc2N2CCCCCC2)C(c2ccn(C)n2)C1] | Enantiomer 2 Rt 12.62 min 99% ee LC-MS: m/z 465.5 (MH+) (2R or 2S)(N-(3-(azepan-1-yl)-4-(4-methyl-2-(1-methyl-1H-pyrazol-3- yl)piperazine-1-carbonyl)phenyl)cyclopropane carboxamide Example 71b |

Preparation: N-(3-(azepan-1-yl)-4-(2-phenyl-4-propylpiperazine-1-carbonyl)phenyl) Cyclopropanecarboxamide

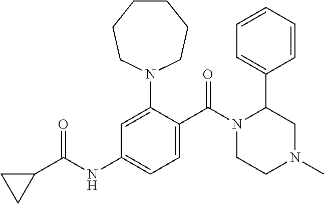 [SMILES: CN1CCN(C(=O)c2ccc(NC(=O)C3CC3)cc2N2CCCCCC2)C(c2ccccc2)C1]

### Examples 72a and 72b

The synthesis of the title compound was effected analogously to the synthesis of the compound of Example 69a-c (N-(3-(azepan-1-yl)-4-(4-methyl-2-(thiophen-2-yl)piperazine-1-carbonyl)phenyl) Cyclopropane carboxamide) wherein N-[3-(azepan-1-yl)-4-(2-phenylpiperazine-1-carbonyl)phenyl]cyclopropanecarboxamide was used instead of N-[3-(azepan-1-yl)-4-(2-thiophen-2-ylpiperazine-1-carbonyl)phenyl]cyclopropanecarboxamide. The title compound was obtained in a yield of 70% (80 mg).

1H NMR (400 MHz, DMSO-d6) δ ppm 10.27-9.95 (m, 1H), 7.81-6.76 (m, 8H), 5.89-4.49 (m, 1 H), 3.39-2.87 (m, 4 H), 2.15 (br s, 3 H), 4.46-1.86 (m, 6H), 1.83-1.67 (m, 1H), 1.84-1.25 (m, 8 H), 0.88-0.67 (m, 4H); LC-MS: m/z 461.4 (MH+). The racemic mixture was then separated into single enantiomers by preparative HPLC

| | Column | Chiralpak IC (25 × 0.46 cm), 5 u | |
|---|---|---|---|
| | Mobile phase | n-Hexane/(Ethanol + 0.1% | |
| | | isopropylamine) 75/25% v/v | |
| | Flow rate (ml/min) | 17 | ml/min |
| | DAD detection | 220 | nm |
| Preparative chiral | Loop | 500 | μL |
| chromatography | Total amount | 41 | mg |
| protocol: | Injection | 8 | mg (each injection) |

| | |
|---|---|
| 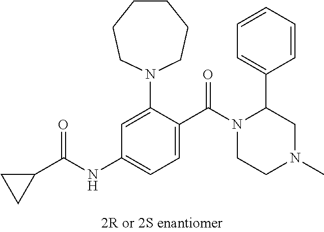 [SMILES: CN1CCN(C(=O)c2ccc(NC(=O)C3CC3)cc2N2CCCCCC2)C(c2ccccc2)C1] | Enantiomer 1 Rt 5.6 min 99% ee LC-MS: m/z 461.4 (MH+) (2R or 2S) N-(3-(azepan-1-yl)-4-(2-phenyl-4-propylpiperazine-1- carbonyl)phenyl) Cyclopropanecarboxamide Example 72a |
| 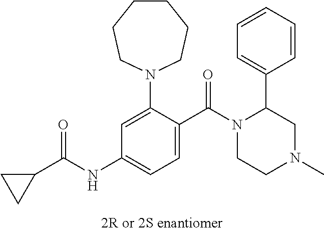 [SMILES: CN1CCN(C(=O)c2ccc(NC(=O)C3CC3)cc2N2CCCCCC2)C(c2ccccc2)C1] | Enantiomer 2 Rt 7.65 min 99% ee LC-MS: m/z 461.4 (MH+) (2R or 2S) N-(3-(azepan-1-yl)-4-(2-phenyl-4-propylpiperazine-1- carbonyl)phenyl) Cyclopropanecarboxamide Example 72b |

Preparation: N-[3-(azepan-1-yl)-4-(2-phenyl-4-propylpiperazine-1-carbonyl)phenyl]Cyclopropanecarboxamide

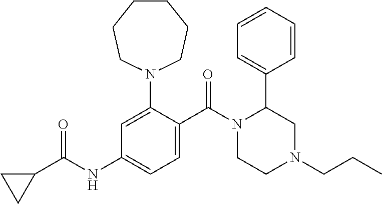 [SMILES: CCCN1CCN(C(=O)c2ccc(NC(=O)C3CC3)cc2N2CCCCCC2)C(c2ccccc2)C1]

### Example 73a, 73b, 73c

To a solution of N-[3-(azepan-1-yl )-4-(2-phenylpiperazine-1-carbonyl)phenyl]cyclopropanecarboxamide (110.0 mg, 0.250 mmol) and N,N-Diisopropylethylamine (0.06 mL, 0.370 mmol) in MeCN (1.5 mL), 1-bromopropane (45.44 mg, 0.370 mmol) was added and the reaction was stirred at 60 C overnight. The day after the reaction was concentrated to dryness and the residue was taken up in AcOEt. The organic phase was washed with a s.s. of NaHCO₃, Brine, dried over Na₂SO₄, filtered and concentrated under vacuum. The residue was purified by FC NH (eluting from 100% of cHex to cHex/AcOEt 6:4) and it was further purified by FC on RP (eluting from water/CH₃CN 95:5 to water/CH₃CN 5:95) affording the product of formula (N-[3-(azepan-1-yl)-4-(2-phenyl-4-propylpiperazine-1-carbonyl)phenyl]Cyclopropanecarboxamide).
Yield: 72 mg

1H NMR (400 MHz, DMSO-d6) δ ppm 10.36-9.79 (m, 1 H), 8.12-6.60 (m, 8 H), 5.93-4.45 (m, 1 H), 3.62-3.35 (m, 1 H) 3.35-2.83 (4 H), 4.44-2.82 (m, 2 H), 2.81-2.62 (m, 1 H), 2.43-2.28 (m, 1 H), 2.29-2.19 (m, 2 H), 2.11-1.87 (m, 1 H), 1.80-1.66 (m, 1 H), 1.51-1.38 (m, 2 H), 1.85-1.23 (m, 8 H), 0.94-0.72 (m, 7 H); LC-MS: m/z 489.4 (MH+).

The racemic mixture (Example 73c) was then separated into single enantiomers by preparative HPLC

| | Column | Chiralpak IC (25 × 2.0 cm), 5 μ | |
|---|---|---|---|
| | Mobile phase | Methanol + 0.1% | |
| | | isopropylamine) 30% | |
| | Flow rate (ml/min) | 17 | ml/min |
| | DAD detection | 220 | nm |
| Preparative chiral | Loop | 500 | μL |
| chromatography | Total amount | 66 | mg |
| protocol: | Injection | 11 | mg (each injection) |

| | |
|---|---|
| 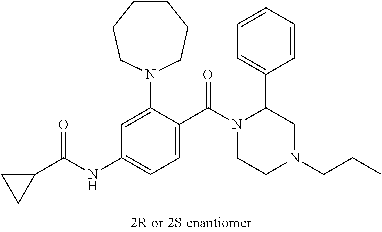 [SMILES: CCCN1CCN(C(=O)c2ccc(NC(=O)C3CC3)cc2N2CCCCCC2)C(c2ccccc2)C1] | Enantiomer 1 Rt 7.85 min 99% ee LC-MS: m/z 489.4 (MH+) (2R or 2S) N-[3-(azepan-1-yl)-4-(2-phenyl-4-propylpiperazine-1- carbonyl)phenyl] Cyclopropanecarboxamide Example 73a |
| 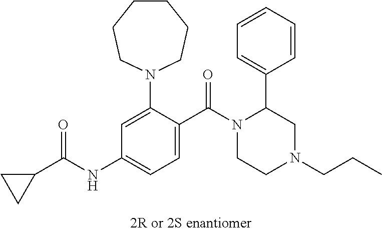 [SMILES: CCCN1CCN(C(=O)c2ccc(NC(=O)C3CC3)cc2N2CCCCCC2)C(c2ccccc2)C1] | Enantiomer 2 Rt 10.47 min 99% ee LC-MS: m/z 489.4 (MH+) (2R or 2S) N-[3-(azepan-1-yl)-4-(2-phenyl-4-propylpiperazine-1- carbonyl)phenyl] Cyclopropanecarboxamide Example 73b |

Preparation: N-[3-(azepan-1-yl)-4-(4-methyl-2-pyridin-2-ylpiperazine-1-carbonyl)phenyl]cyclopropanecarboxamide

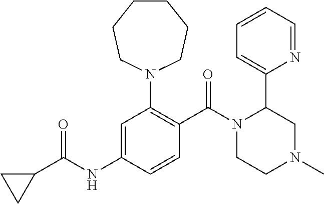 [SMILES: CN1CCN(C(=O)c2ccc(NC(=O)C3CC3)cc2N2CCCCCC2)C(c2ccccn2)C1]

### Example 74

The synthesis of the title compound was effected analogously to the synthesis of the compound of Example 69a-c (N-(3-(azepan-1-yl)-4-(4-methyl-2-(thiophen-2-yl)piperazine-1-carbonyl)phenyl) cyclopropane carboxamide) wherein N-[3-(azepan-1-yl)-4-(2-pyridin-2-ylpiperazine-1-carbonyl)phenyl]cyclopropanecarboxamide was used instead of N-[3-(azepan-1-yl)-4-(2-thiophen-2-ylpiperazine-1-carbonyl)phenyl]cyclopropanecarboxamide. The title compound was obtained in a yield of 70% (21 mg).

1H NMR (500 MHz, DMSO-d6) δ ppm 10.28-9.92 (m, 1 H), 8.70-8.37 (m, 1 H), 7.88-7.65 (m, 1 H), 7.55-6.68 (m, 5 H), 5.72-4.54 (m, 1 H), 2.10 (s, 3 H), 4.53-1.86 (m, 10 H), 1.85-1.36 (m, 9 H), 0.92-0.63 (m, 4 H); LC-MS: m/z 462.3 (MH+).

Preparation: N-[3-(azepan-1-yl)-4-[4-methyl-2-(trifluoromethyl)piperazine-1-carbonyl]phenyl]Cyclopropanecarboxamide

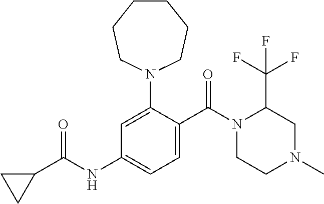 [SMILES: CN1CCN(C(=O)c2ccc(NC(=O)C3CC3)cc2N2CCCCCC2)C(C(F)(F)F)C1]

### Example 75

The synthesis of the title compound was effected analogously to the synthesis of the compound of Example 69a-c (N-(3-(azepan-1-yl)-4-(4-methyl-2-(thiophen-2-yl)piperazine-1-carbonyl)phenyl) cyclopropane carboxamide) wherein N-[3-(azepan-1-yl)-4-[2-(trifluoromethyppiperazine-1-carbonyl]phenyl]cyclopropanecarboxamide was used instead of N-[3-(azepan-1-yl)-4-(2-thiophen-2-ylpiperazine-1-carbonyl)phenyl]cyclopropanecarboxamide. The title compound was obtained in a yield of 13% (4.4 mg). 1H NMR (500 MHz, DMSO-d6) δ ppm 10.31-10.07 (m, 1H), 7.52-7.33 (m, 1H), 6.98 (br s, 1 H), 6.98-6.79 (m, 1H), 5.34-3.86 (m, 1H), 3.32 (s, 2 H), 3.27-3.18 (m, 4H), 2.23-2.14 (m, 3H), 2.50 (s, 4 H), 1.77-1.40 (m, 9 H), 0.89-0.72 (m, 4 H); LC-MS: m/z 453.4 (MH+).

Preparation: N-[3-(azepan-1-yl)-4-(2-benzyl-4-methylpiperazine-1-carbonyl)phenyl]Cyclopropanecarboxamide

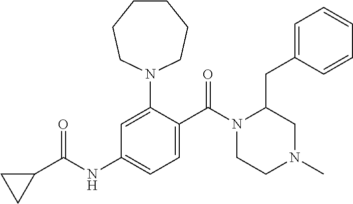 [SMILES: CN1CCN(C(=O)c2ccc(NC(=O)C3CC3)cc2N2CCCCCC2)C(Cc2ccccc2)C1]

### Example 76

The synthesis of the title compound was effected analogously to the synthesis of the compound of Example 69a-c (N-(3-(azepan-1-yl )-4-(4-methyl-2-(thiophen-2-yl)piperazine-1-carbonyl)phenyl) Cyclopropane carboxamide) wherein N-[3-(azepan-1-yl )-4-(2-benzylpiperazine carbonyl)phenyl]cyclopropanecarboxamide was used instead of N-[3-(azepan-1-yl )-4-(2-thiophen ylpiperazine-1-carbonyl)phenyl]cyclopropanecarboxamide. The title compound was obtained in a yield of 40% (27 mg). 1H NMR (500 MHz, DMSO-d6) δ ppm 10.23-9.99 (m, 1H), 7.46-6.21 (m, 8 H), 4.89-3.40 (m, 1H), 3.25-3.17 (m, 4 H) 2.20-2.10 (m, 3 H), 4.49-1.79 (m, 8 H), 1.78-1.36 (m, 9 H), 0.97-0.69 (m, 4H); LC-MS: 475.3 (MH+).

Preparation: N-[3-(azepan-1-yl)-4-[2-(hydroxymethyl)-4-methylpiperazine-1-carbonyl]phenyl]cyclopropanecarboxamide

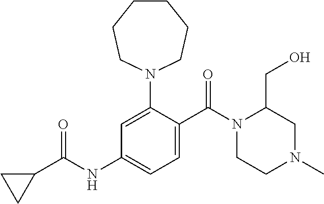 [SMILES: CN1CCN(C(=O)c2ccc(NC(=O)C3CC3)cc2N2CCCCCC2)C(CO)C1]

### Example 77

The synthesis of the title compound was effected analogously to the synthesis of the compound of Example 69a-c (N-(3-(azepan-1-yl)-4-(4-methyl-2-(thiophen-2-yl)piperazine-1-carbonyl)phenyl) Cyclopropane carboxamide) wherein N-[3-(azepan-1-yl)-4-[2-(hydroxymethyppiperazine-1-carbonyl]phenyl]cyclopropanecarboxamide was used instead of N-[3-(azepan-1-yl)-4-(2-thiophen-2-ylpiperazine-1-carbonyl)phenyl]cyclopropanecarboxamide. The title compound was obtained in a yield of 12.3% (9.2 mg). 1H NMR (500 MHz, DMSO-d6) δ ppm 10.19-10.05 (M, 1 H), 7.39-7.27 (m, 1H), 7.08-6.80 (m, 2 H), 4.90-4.56 (m, 1H), 4.90-4.56 (m, 1 H), 3.84-3.38 (m, 2 H), 4.51-3.32 (m, 1H) 4.33-3.18 (m, 1H), 3.27-3.17 (m, 4H), 3.11-2.83 (m, 1H), 2.23-2.08 (m, 3H), 3.14-1.77 (m, 4H), 1.75-1.43 (m, 9 H), 0.85-0.71 (m, 4 H); LC-MS: 415.3 (MH+).

Preparation: N-[3-(azepan-1-yl)-4-(2,4-dimethylpiperazine-1-carbonyl)phenyl]cyclopropanecarboxamide

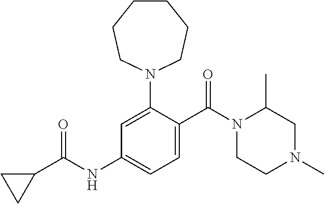 [SMILES: CC1CN(C)CCN1C(=O)c1ccc(NC(=O)C2CC2)cc1N1CCCCCC1]

### Example 78

The synthesis of the title compound was effected analogously to the synthesis of the compound of Example 69a-c (N-(3-(azepan-1-yl)-4-(4-methyl-2-(thiophen-2-yl)piperazine-1-carbonyl)phenyl) Cyclopropane carboxamide) wherein N-[3-(azepan-1-yl)-4-(2-methylpiperazine-1-carbonyl)phenyl]cyclopropanecarboxamide was used instead of N-[3-(azepan-1-yl)-4-(2-thiophen-2-ylpiperazine-1-carbonyl)phenyl]cyclopropanecarboxamide. The title compound was obtained in a yield of 60% (24 mg). ¹ NMR (400 MHz, DMSO-d₆) δ 10.12 (s, 1H), 7.37-7.29 (m, 1H), 7.05-6.98 (m, 1H), 6.95-6.73 (m, 1H), 4.73-3.47 (m, 1H), 3.28-3.11 (m, 6H), 3.02-2.57 (m, 2H), 2.22-1.95 (m, 4H), 1.85-1.42 (m, 10H), 1.27-1.04 (m, 3H), 0.84-0.74 (m, 4H); LC-MS: m/z 399.3 (MH+).

Preparation: N-[3-(azepan-1-yl)-4-(4-methyl-2-propan-2-ylpiperazine-1-carbonyl)phenyl]Cyclopropanecarboxamide

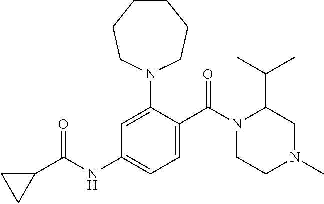 [SMILES: CC(C)C1CN(C)CCN1C(=O)c1ccc(NC(=O)C2CC2)cc1N1CCCCCC1]

### Example 79

The synthesis of the title compound was effected analogously to the synthesis of the compound of Example 69a-c (N-(3-(azepan-1-yl )-4-(4-methyl-2-(thiophen-2-yl)piperazine-1-carbonyl)phenyl) Cyclopropane carboxamide) wherein N-[3-(azepan-1-yl )-4-(2-propan-2-ylpiperazine-1-carbonyl)phenyl]cyclopropanecarboxamide was used instead of N-[3-(azepan-1-yl )-4-(2-thiophen-2-ylpiperazine-1-carbonyl)phenyl]cyclopropanecarboxamide. The title compound was obtained in a yield of 53% (22 mg).

¹ NMR (400 MHz, DMSO-d₆) δ 10.14 (d, OH), 7.41-7.33 (m, 1H), 7.02 (ddd, 1H), 6.94-6.79 (m, 1H), 4.41-4.07 (m, 1H), 3.29-3.16 (m, 6H), 3.16-2.57 (m, 4H), 2.40-2.25 (m, 1H), 1.92-1.43 (m, 12H), 1.00-0.51 (m, 11H); LC-MS: 427.1 (MH+).

Preparation: N-[3-(azepan-1-yl)-4-(4-methyl-2-pyridin-2-ylpiperazine-1-carbonyl)phenyl]

Cyclopropanecarboxamide

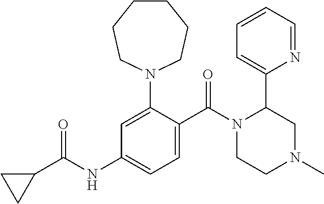 [SMILES: CN1CCN(C(=O)c2ccc(NC(=O)C3CC3)cc2N2CCCCCC2)C(c2ccccn2)C1]

### Example 80

The synthesis of the title compound was effected analogously to the synthesis of the compound of Example 69a-c (N-(3-(azepan-1-yl)-4-(4-methyl-2-(thiophen-2-yl)piperazine-1-carbonyl)phenyl) Cyclopropane carboxamide) wherein N-[3-(azepan-1-yl)-4-(2-pyridin-2-ylpiperazine-1-carbonyl)phenyl]cyclopropanecarboxamide was used instead of N-[3-(azepan-1-yl)-4-(2-thiophen-2-ylpiperazine-1-carbonyl)phenyl]cyclopropanecarboxamide. The title compound was obtained in a yield of 70% (21.5 mg).

1H NMR (500 MHz, DMSO-d6) δ ppm 10.28-9.92 (m, 1 H), 8.70-8.37 (m, 1 H), 7.88-7.65 (m, 1H), 7.55-6.68 (m, 5 H), 5.72-4.54 (m, 1H), 4.53-1.86 (m, 10 H), 2.10 (s, 3 H), 1.85-1.36 (m, 9 H), 0.92-0.63 (m, 4 H); LC-MS: m/z 462.3 (MH+).

Preparation: N-[3-(azepan-1-yl)-4-[4-[(3,5-dimethyl-1,2-oxazol-4-yl)methyl]-2-phenylpiperazine-1-carbonyl]phenyl]cyclopropanecarboxamide

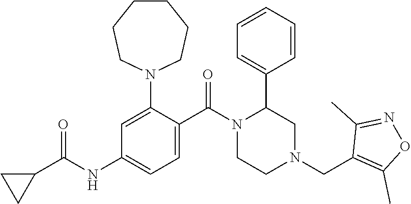 [SMILES: Cc1noc(C)c1CN1CCN(C(=O)c2ccc(NC(=O)C3CC3)cc2N2CCCCCC2)C(c2ccccc2)C1]

### Example 81

To a solution of N-[3-(azepan-1-yl)-4-(2-phenylpiperazine-1-carbonyl)phenyl]cyclopropanecarboxamide (55.0 mg, 0.120 mmol) in MeCN (3.5 mL), N,N-Diisopropylethylamine (0.04 mL, 0.250 mmol) followed by 3,5-dimethyl-4-isoxazolecarbaldehyde (23.12 mg, 0.180 mmol) were added. The reaction mixture was stirred for 15 min before adding sodium triacetoxyborohydride (52.2 mg, 0.250 mmol). The resulting mixture was stirred at RT ON. The day after the mixture was diluted with AcOEt, washed with Brine, dried over Na₂SO₄, filtered and concentrated under vacuum. The residue was purified by FC on NH column (eluting from 100% of cHex to cHex/AcOEt 3: 7) affording the product of formula N-[3-(azepan-1-yl)-4-[4-[(3,5-dimethyl-1,2-oxazol-4-yOmethyl]-2-phenylpiperazine-1-carbonyl]phenyl] cyclopropanecarboxamide
Yield: 32 mg

¹ NMR (400 MHz, DMSO-d₆) δ 10.13 (t, 1H), 7.71-7.40 (m, 1H), 7.37-7.18 (m, 4H), 7.13-7.00 (m, 1H), 6.96-6.85 (m, 1H), 5.75 (d, 1H), 4.90-4.17 (m, 1H), 3.50-3.19 (m, 4H), 3.11-2.61 (m, 4H), 2.44-1.95 (m, 9H), 1.86-1.22 (m, 10H), 0.78 (d, 4H); LC-MS: m/z 556.4 (MH+).

Preparation: N-[3-(azepan-1-yl)-4-[4-(1,2-oxazol-3-ylmethyl)-2-phenylpiperazine-1-carbonyl]phenyl]Cyclopropanecarboxamide

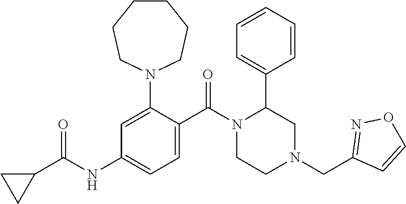 [SMILES: O=C(Nc1ccc(C(=O)N2CCN(Cc3ccon3)CC2c2ccccc2)c(N2CCCCCC2)c1)C1CC1]

### Example 82

The synthesis of the title compound was effected analogously to the synthesis of the compound of Example 81 (N-[3-(azepan-1-yl)-4-[4-[(3,5-dimethyl-1,2-oxazol-4-yl)methyl]-2-phenylpiperazine-1-carbonyl]phenyl] cyclopropanecarboxamide) wherein 1,2-oxazole-3-carbaldehyde was used instead of 3,5-dimethyl-4-isoxazolecarbaldehyde The title compound was obtained in a yield of 20% (7 mg).

¹ NMR (400 MHz, DMSO-d₆) δ 10.13 (t, 1H), 8.93-8.83 (m, 1H), 7.77-6.82 (m, 8H), 6.62-6.31 (m, 1H), 5.82-5.67 (m, 1H), 4.62-3.44 (m, 3H), 3.30-3.27 (m, 2H), 3.21-2.66 (m, 4H), 2.45-2.05 (m, 2H), 1.86-1.16 (m, 10H), 0.85-0.62 (m, 4H); LC-MS: m/z 528.3 (MH+).

Preparation: N-[3-(azepan-1-yl)-4-[4-(1,3-oxazol-2-ylmethyl)-2-phenylpiperazine-1-carbonyl]phenyl]cyclopropanecarboxamide

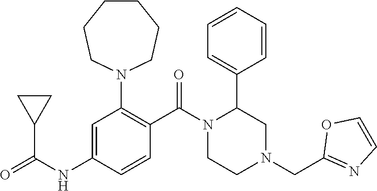 [SMILES: O=C(Nc1ccc(C(=O)N2CCN(Cc3ncco3)CC2c2ccccc2)c(N2CCCCCC2)c1)C1CC1]

### Example 83

The synthesis of the title compound was effected analogously to the synthesis of the compound of Example 81 (N-[3-(azepan-1-yl)-4-[4-[(3,5-dimethyl-1,2-oxazol-4-yl)methyl]-2-phenylpiperazine-1-carbonyl]phenyl]cyclopropanecarboxamide) wherein 2-oxazolecarboxaldheyde was used instead of 3,5-dimethyl-4-isoxazolecarbaldehyde The title compound was obtained in a yield of 42% (15 mg).

¹ NMR (400 MHz, DMSO-d₆) δ 10.25-10.06 (m, 1H), 8.21-8.04 (m, 1H), 7.83-7.11 (m, 7H), 7.07-6.84 (m, 2H), 5.85-4.28 (m, 1H), 3.87-3.37 (m, 2H), 3.28-3.23 (m, 2H), 3.19-2.61 (m, 4H), 2.39-2.16 (m, 1H), 1.92-1.28 (m, 11H), 0.78 (d, 5H); LC-MS: m/z 528.3 (MH+)

Preparation: N-[4-[2-(2-fluorophenyl)-4-methylpiperazine-1-carbonyl]-3-pyrrolidin-1-ylphenyl]cyclopropanecarboxamide

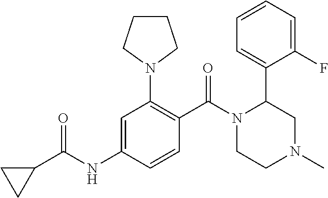 [SMILES: CN1CCN(C(=O)c2ccc(NC(=O)C3CC3)cc2N2CCCC2)C(c2ccccc2F)C1]

### Example 84a, 84b, 84c

To a solution of N-[4-[2-(2-fluorophenyl)piperazine-1-carbonyl]-3-pyrrolidin-1-ylphenyl]cyclopropanecarboxamide (95.0 mg, 0.220 mmol) in Methanol (5 mL) was added formaldehyde (176.63 mg, 2.18 mmol) and sodium triacetoxyborohydride (276.75 mg, 1.31 mmol). The reaction was stirred at room temperature ON. The day after the reaction was concentrated to dryness under reduced pressure and purified by SCX washing with MeOH and eluting with 1N NH₃ in MeOH. Basic fractions were evaporated and the residue was purified by FC on NH column (eluting from 100% of cHex to cHex/AcOEt 1:1) affording the product of formula N-[4-[2-(2-fluorophenyl)-4-methylpiperazine-1-carbonyl]-3-pyrrolidin-1-ylphenyl]cyclopropanecarboxamide as a racemic mixture.
Yield: 54 mg

1H NMR (500 MHz, DMSO-d6) δ ppm 10.36-9.83 (m, 1 H), 8.06-7.40 (m, 1 H), 7.39-7.25 (m, 1 H), 7.24-6.41 (m, 5 H), 6.07-4.84 (m, 1 H), 4.65-2.64 (m, 8 H), 2.42-2.23 (m, 1 H), 2.21-2.09 (m, 3 H), 1.80-1.71 (m, 1 H), 2.06-1.42 (m, 5 H), 0.87-0.66 (m, 4 H); LC-MS: 451.19 (MH+). The racemic mixture (Example 84c) was then separated into single enantiomers by preparative HPLC

| | Column | Chiralpak AS-H (25 × 2.0 cm), 5 μ | |
|---|---|---|---|
| | Mobile phase | n-Hexane/Ethanol 85/15% v/v | |
| | Flow rate (ml/min) | 17 | ml/min |
| | DAD detection | 250 | nm |
| Preparative chiral | Loop | 500 | μL |
| chromatography | Total amount | 49.5 | mg |
| protocol: | Injection | 13.7 | mg (each injection) |

| | |
|---|---|
| 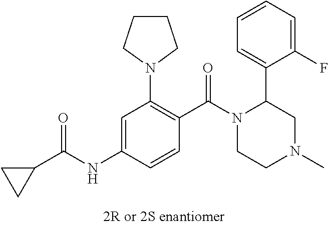 [SMILES: CN1CCN(C(=O)c2ccc(NC(=O)C3CC3)cc2N2CCCC2)C(c2ccccc2F)C1] | Enantiomer 1 Rt 6.99 min 100% ee LC-MS: m/z 451.19 (MH+) (2R or 2S) N-[4-[2-(2-fluorophenyl)-4-methylpiperazine-1-carbonyl]-3- pyrrolidin-1-ylphenyl]cyclopropanecarboxamide Example 84a |
| 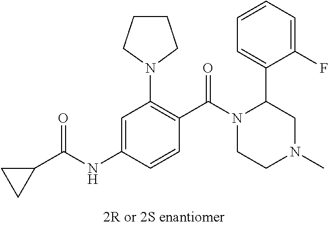 [SMILES: CN1CCN(C(=O)c2ccc(NC(=O)C3CC3)cc2N2CCCC2)C(c2ccccc2F)C1] | Enantiomer 2 Rt 13.60 min 100% ee LC-MS: m/z 451.19 (MH+) (2R or 2S) N-[4-[2-(2-fluorophenyl)-4-methylpiperazine-1-carbonyl]-3- pyrrolidin-1-ylphenyl]cyclopropanecarboxamide Example 84b |

Preparation: N-[3-(6-azaspiro[3.4]octan-6-yl)-4-(4-methyl-2-thiophen-2-ylpiperazine-1-carbonyl)phenyl]Cyclopropanecarboxamide

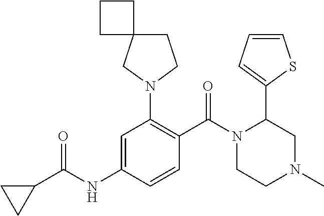 [SMILES: CN1CCN(C(=O)c2ccc(NC(=O)C3CC3)cc2N2CCC3(CCC3)C2)C(c2cccs2)C1]

### Example 85

The synthesis of the title compound was effected analogously to the synthesis of the compound of Example 84a-c (N-[4-[2-(2-fluorophenyl)-4-methylpiperazine-1-carbonyl]-3-pyrrolidin-1-ylphenyl]cyclopropanecarboxamide) wherein N-[3-(6-azaspiro[3.4]octan-6-yl)-4-(2-thiophen ylpiperazine-1-carbonyl)phenyl]cyclopropanecarboxamide was used instead of N-[4-[2-(2-fluorophenyppiperazine-1-carbonyl]-3-pyrrolidin-1-ylphenyl]cyclopropanecarboxamide. The title compound was obtained in a yield of 17% (2.5 mg) as a racemic mixture.

H NMR (500 MHz, METHANOL-d4) δ ppm 7.40-7.27 (m, 2 H), 7.07-7.03 (m, 1 H), 6.90-6.82 (m, 1 H), 7.19-6.59 (m, 1 H), 7.48-6.56 (m, 1 H), 6.17-4.97 (m, 1 H), 4.55-3.35 (m, 2 H), 3.06-3.02 (m, 1 H), 3.00-2.86 (m, 1 H), 2.85-2.73 (m, 1 H), 2.67-2.57 (m, 1 H), 4.64-2.39 (m, 2 H), 2.51-2.38 (m, 1 H), 2.35-2.26 (m, 3 H), 2.12-1.94 (m, 1 H), 1.81-1.65 (m, 1 H), 2.22-1.58 (m, 8 H), 1.00-0.81 (m, 4 H), LC-MS: m/z 479.2 (MH+).

Preparation: N-[4-(4-methyl-2-thiophen-2-ylpiperazine-1-carbonyl)-3-pyrrolidin-1-ylphenyl]cyclopropanecarboxamide

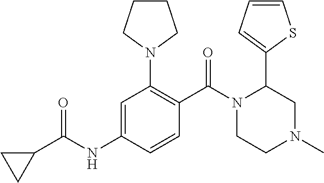 [SMILES: CN1CCN(C(=O)c2ccc(NC(=O)C3CC3)cc2N2CCCC2)C(c2cccs2)C1]

### Example 86a, 86b, 86c

The synthesis of the title compound was effected analogously to the synthesis of the compound of Example 84a-c (N-[4-[2-(2-fluorophenyl)-4-methylpiperazine-1-carbonyl]-3-pyrrolidin-1-ylphenyl]cyclopropanecarboxamide) wherein N-[3-pyrrolidin-1-yl-4-(2-thiophen-2-ylpiperazine-1-carbonyl)phenyl]cyclopropanecarboxamide was used instead of N-[4-[2-(2-fluorophenyppiperazine-1-carbonyl]-3-pyrrolidin-1-ylphenyl]cyclopropanecarboxamide. The title compound was obtained in a yield of 5.3% (15.9 mg) as a racemic mixture. 1H NMR (500 MHz, DMSO-d6) δ 10.26-10.01 (m, 1 H), 7.45-7.32 (m, 1 H), 7.27-6.55 (m, 5 H), 6.05-4.83 (m, 1 H), 4.39-2.61 (m, 8 H), 2.40-2.16 (m, 4 H), 2.06-1.82 (m, 3 H), 1.80-1.71 (m, 1 H), 1.71-1.52 (m, 2 H), 0.83-0.72 (m, 4 H); LC-MS: m/z 437.25 (MH+).

The racemic mixture (Example 86c) was then separated into single enantiomers by preparative HPLC

| | Column | Chiralcel OD-H (25 × 2.0 cm), 5 μ | |
|---|---|---|---|
| | Mobile phase | n-Hexane/Ethanol 88/12% v/v | |
| | Flow rate (ml/min) | 17 | ml/min |
| | DAD detection | 220 | nm |
| Preparative chiral | Loop | 1000 | μL |
| chromatography | Total amount | 12.5 | mg |
| protocol: | Injection | 6.2 | mg (each injection) |

| | |
|---|---|
| 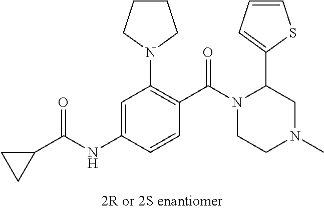 [SMILES: CN1CCN(C(=O)c2ccc(NC(=O)C3CC3)cc2N2CCCC2)C(c2cccs2)C1] | Enantiomer 1 Rt 8.42 min 100% ee LC-MS: m/z 437.25 (MH+) (2R or 2S) N-[4-(4-methyl-2-thiophen-2-ylpiperazine-1-carbonyl)-3- pyrrolidin-1-ylphenyl]cyclopropanecarboxamide Example 86a |
| 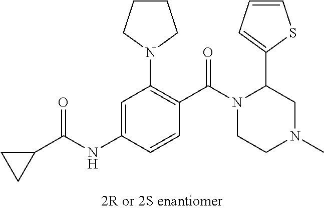 [SMILES: CN1CCN(C(=O)c2ccc(NC(=O)C3CC3)cc2N2CCCC2)C(c2cccs2)C1] | Enantiomer 2 Rt 10.48 min 94.2% ee LC-MS: m/z 437.25 (MH+) (2R or 2S) N-[4-(4-methyl-2-thiophen-2-ylpiperazine-1-carbonyl)-3- pyrrolidin-1-ylphenyl]cyclopropanecarboxamide Example 86b |

Preparation: N-[4-[4-methyl-2-(1-methylpyrazol-4-yl)piperazine-1-carbonyl]-3-pyrrolidin-1-ylphenyl]Cyclopropanecarboxamide

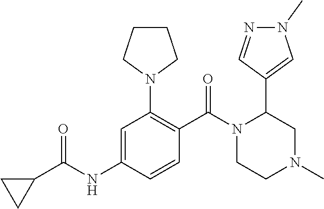 [SMILES: CN1CCN(C(=O)c2ccc(NC(=O)C3CC3)cc2N2CCCC2)C(c2cnn(C)c2)C1]

### Example 87

The synthesis of the title compound was effected analogously to the synthesis of the compound of Example 84a-c (N-[4-[2-(2-fluorophenyl)-4-methylpiperazine-1-carbonyl]-3-pyrrolidin-1-ylphenyl]cyclopropanecarboxamide) wherein N-[4-[2-(1-methylpyrazol-4-yppiperazine-1-carbonyl]-3-pyrrolidin-1-ylphenyl]cyclopropanecarboxamide was used instead of N-[4-[2-(2-fluorophenyl)piperazine-1-carbonyl]-3-pyrrolidin-1-yl phenyl]cyclopropanecarboxamide. The title compound was obtained in a yield of 30% (27 mg) as a racemic mixture.

1H NMR (400 MHz, DMSO-d6) δ ppm 10.23-9.99 (m, 1 H), 7.80-7.50 (m, 1 H), 7.48-7.01 (m, 1 H), 7.15-6.68 (m, 3 H), 5.70-4.50 (m, 1 H), 3.89-3.67 (m, 3 H), 4.38-2.57 (m, 8 H), 2.29-2.07 (m, 4 H), 2.04-1.57 (m, 6 H), 0.87-0.68 (m, 4 H), LC-MS: m/z 437.23 (MH+).

Preparation: N-[4-[2-(3-fluorophenyl)-4-methylpiperazine-1-carbonyl]-3-pyrrolidin-1-ylphenyl]cyclopropanecarboxamide

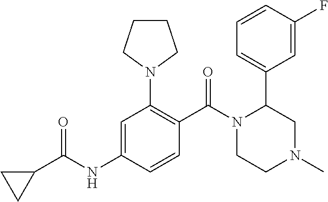 [SMILES: CN1CCN(C(=O)c2ccc(NC(=O)C3CC3)cc2N2CCCC2)C(c2cccc(F)c2)C1]

### Example 88a, 88b, 88c

To a mixture of N-[4-[2-(3-fluorophenyppiperazine-1-carbonyl]-3-pyrrolidin-1-ylphenyl]cyclopropanecarboxamide (72.0 mg, 0.160 mmol) and potassium carbonate (42.86 mg, 0.310 mmol) and DMF (1 mL) a solution of 4-methylbenzenesulfonic acid methyl ester (28.87 mg, 0.160 mmol) in DMF (0.500 mL) was added and the reaction was stirred at RT ON. The day after the reaction as poured into a s. s. of NaHCO₃ and AcOEt. The 2 Phases were separated and the organic one was washed with Brine, dried over Na₂SO₄, filtered and concentrated to dryness under vacuum. The residue was purified by SCX washing with MeOH and eluting with 1N NH₃ in MeOH. Basic fractions were evaporated and the residue was purified by FC on NH column (eluting from 100% of cHex to cHex/AcOEt 1:1) affording the product of formula N-[4-[2-(3-fluorophenyl)-4-methylpiperazine-1-carbonyl]-3-pyrrolidin-1-ylphenyl]cyclopropanecarboxamide.
Yield: 31 mg

1H NMR (500 MHz, DMSO-d6) δ 10.20-10.05 (m, 1 H), 7.59-6.99 (m, 2 H), 7.18-6.79 (m, 2 H), 7.61-6.76 (m, 3 H), 5.83-4.65 (m, 1 H), 4.50-3.25 (m, 1 H), 3.30-2.94 (m, 4 H), 2.82-2.57 (m, 1 H), 3.48-2.54 (m, 2 H), 2.39-2.22 (m, 1 H), 2.27-2.13 (m, 3 H), 2.05-1.85 (m, 1 H), 1.80-1.71 (m, 1 H), 1.98-1.59 (m, 4 H), 0.86-0.68 (m, 4 H); LC-MS: m/z 451.16(MH+).

The racemic mixture (Example 88c) was then separated into single enantiomers by preparative HPLC

| | Column | Chiralpak AS-H (25 × 2.0 cm), 5 μ | |
|---|---|---|---|
| | Mobile phase | n-Hexane/Ethanol 85/15% v/v | |
| | Flow rate (ml/min) | 17 | ml/min |
| | DAD detection | 220 | nm |
| Preparative chiral | Loop | 500 | μL |
| chromatography | Total amount | 27.5 | mg |
| protocol: | Injection | 7 | mg (each injection) |

| | |
|---|---|
| 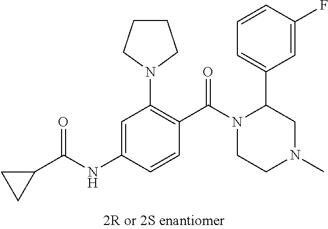 [SMILES: CN1CCN(C(=O)c2ccc(NC(=O)C3CC3)cc2N2CCCC2)C(c2cccc(F)c2)C1] | Enantiomer 1 Rt 7.16 min 100% ee LC-MS: m/z 451.16 (MH+) (2R or 2S) N-[4-[2-(3-fluorophenyl)-4-methylpiperazine-1-carbonyl]-3- pyrrolidin-1-ylphenyl]cyclopropanecarboxamide Example 88a |
| 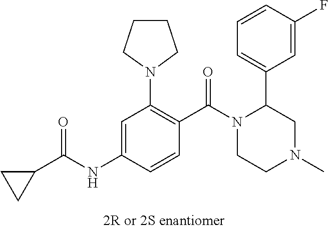 [SMILES: CN1CCN(C(=O)c2ccc(NC(=O)C3CC3)cc2N2CCCC2)C(c2cccc(F)c2)C1] | Enantiomer 2 Rt 10.64 min 100% ee LC-MS: m/z 451.16 (MH+) (2R or 2S) N-[4-[2-(3-fluorophenyl)-4-methylpiperazine-1-carbonyl]-3- pyrrolidin-1-ylphenyl]cyclopropanecarboxamide Example 88b |

Preparation: N-[4-[2-(4-fluorophenyl)-4-methylpiperazine-1-carbonyl]-3-pyrrolidin-1ylphenyl]cyclopropanecarboxamide

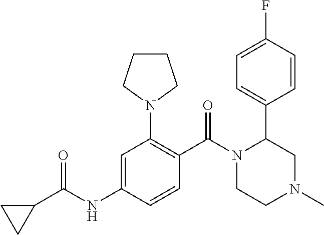 [SMILES: CN1CCN(C(=O)c2ccc(NC(=O)C3CC3)cc2N2CCCC2)C(c2ccc(F)cc2)C1]

### Example 89

The synthesis of the title compound was effected analogously to the synthesis of the compound of Example 88a-c (N-[4-[2-(3-fluorophenyl)-4-methylpiperazine-1-carbonyl]-3-pyrrolidin-1-ylphenyl]cyclopropanecarboxamide) wherein N-[4-[2-(4-fluorophenyl)piperazine-1-carbonyl]-3-pyrrolidin-1-ylphenyl]cyclopropanecarboxamide was used instead of N-[4-[2-(3-fluorophenyl)piperazine-1-carbonyl]-3-pyrrolidin-1-ylphenyl]cyclopropanecarboxamide. The title compound was obtained in a yield of 48% (42 mg) as a racemic mixture.

1H NMR (400 MHz, DMSO-d6) δ 10.10 (d, 1H), 7.90-7.37 (m, 1H), 7.28-7.12 (m, 3H), 7.02-6.76 (m, 3H), 5.89-4.25 (m, 1H), 3.09-2.63 (m, 8H), 2.38-2.15 (m, 3H), 1.91 (s, 4H), 1.68 (d, 3H), 0.76 (d, 4H); LC-MS: m/z 451.24 (MH+)

### Examples of compounds varying at the R1 group may be prepared using Synthetic Scheme 4.

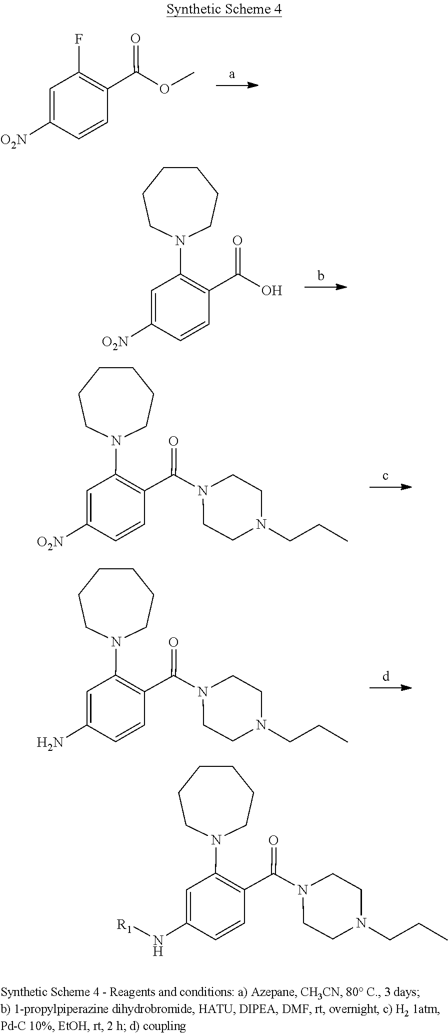

Preparation: 2-(azepan-1-yl)-4-nitrobenzoic acid hydrochloride

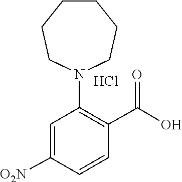 [SMILES: O=C(O)c1ccc([N+](=O)[O-])cc1N1CCCCCC1 ; Cl]

A mixture of 2-fluoro-4-nitrobenzoic acid (4.0 g, 21.61 mmol) and Homopiperidine (12.18 mL, 108.04 mmol) in MeCN (40 mL) was stirred at 80° C. for 4 days. The reaction was concentrated under vacuum and the residue was taken up with HCI 1N and the suspension was filtered. The solid was then washed with H₂O and dried under vacuum affording the product of formula 2-(azepan-1-yl)-4-nitrobenzoic acid hydrochloride. The product was used in the next step without further purification.
Yield: 5.14 g

¹ NMR (400 MHz, DMSO-d₆) δ 14.11 (bs, 1H), 7.81 (d, 1H), 7.72 (d, 1H), 7.59 (dd, 1H), 3.71 (bs, 1H), 3.44-3.32 (m, 4H), 1.79 (s, 4H), 1.57 (dq, 4H); LC-MS: m/z 265.1 (MH+)

Preparation: 1-[5-nitro-2-(4-propylpiperazine-1- carbonyl)phenyl]azepane

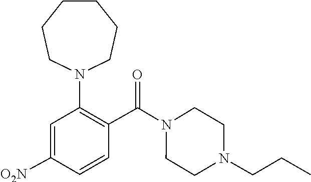 [SMILES: CCCN1CCN(C(=O)c2ccc([N+](=O)[O-])cc2N2CCCCCC2)CC1]

To a mixture of 2-(azepan-1-yl )-4-nitrobenzoic acid hydrochloride (650 mg, 2.16 mmol) and [dimethylamino(3-triazolo[4,5-b]pyridinyloxy)methylidene]-dimethylammonium;hexafluorophosphate (821.8 mg, 2.16 mmol) in DMF (21.8 mL) N,N-Diisopropylethylamine (0.38 mL, 2.16 mmol) was added. The reaction was stirred at RT for 30 min and after this time a solution of N,N-Diisopropylethylamine (0.75 mL, 4.32 mmol) and 1-propylpiperazine dihydrobromide (626.87 mg, 2.16 mmol) in DMF (3 mL) was added. The reaction mixture was stirred at RT ON. The day after the reaction was diluted with a s.s. of NaHCO₃ and extracted with AcOEt 3 times. The organic phase was then washed with Brine, dried over Na₂SO₄, filtered and concentrated under vacuum. The residue was purified by FC on NH column (eluting from cHex/AcOEt 95:5 to cHex/AcOEt 1:1) affording the compound of formula ([2-(azepan-1-yl)-4-nitrophenyI]-(4-propylpiperazin-1-yl)methanone).
Yield: 870 mg

¹ NMR (400 MHz, DMSO-d₆) δ 7.63 (d, 1H), 7.57 (dd, 1H), 7.28 (d, 1H), 3.72-3.52 (m, 2H), 3.42-3.31 (m, 2H), 3.22-3.14 (m, 2H), 2.46-2.38 (m, 2H), 2.35-2.20 (m, 4H), 1.76-1.70 (m, 4H), 1.65-1.48 (m, 4H), 1.48-1.39 (m, 4H), 0.86 (t, 3H); LC-MS: m/z 375.3 (MH+)

Preparation: 3-(azepan-1-yl)-4-(4-propylpiperazine-1-carbonyl)aniline

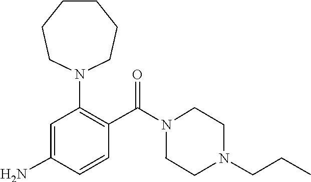 [SMILES: CCCN1CCN(C(=O)c2ccc(N)cc2N2CCCCCC2)CC1]

To a solution of [2-(azepan-1-yl)-4-nitrophenyI]-(4-propylpiperazin-1-yl)methanone (1.2 g, 3.2 mmol) in Ethanol (20 mL) Pd-C (341.02 mg, 0.320 mmol) was added. The reaction was stirred at RT under H₂ atmosphere for 2 h. After this time the reaction was filtered through a pad of celite and concentrated under vacuum affording the product of formula [4-am ino-2-(azepan-1-yl)phenyI]-(4-propylpiperazin-1-yl)methanone.
Yield: 1 g

¹ NMR (400 MHz, DMSO-d₆) δ 6.68 (d, 1H), 6.15 (d, 1H), 6.03 (dd, 1H), 5.10 (s, 2H), 3.54 (d, 2H), 3.23-3.08 (m, 6H), 2.45-2.10 (m, 6H), 1.74-1.32 (m, 10H), 0.85 (t, 3H); LC-MS: m/z 345.3 (MH+)

Preparation: N-(3-(azepan-1-yl)-4-(4-propylpiperazine-1-carbonyl)phenyl)cyclobutanecarboxamide

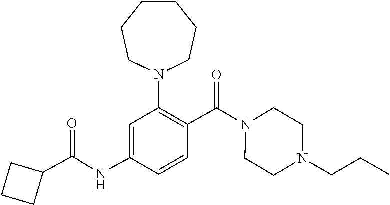 [SMILES: CCCN1CCN(C(=O)c2ccc(NC(=O)C3CCC3)cc2N2CCCCCC2)CC1]

### Example 90

To a mixture of [4-amino-2-(azepan-1-yl)phenyl]-(4-propylpiperazin-1-yl)methanone (100 mg, 0.290 mmol), cyclobutanecarboxylic acid (0.03 mL, 0.350 mmol) and [dimethylamino(3-triazolo[4,5-b]pyridinyloxy)methylidene]-dimethylammonium;hexafluorophosphate (132.45 mg, 0.350 mmol) in DMF (2 mL) N,N-Diisopropylethylamine (56.27 mg, 0.440 mmol) was added and the reaction was stirred at RT ON. The day after s.s. of NaHCO₃ was added to the reaction and it was extracted with AcOEt 3 times. The combined organic phases were then washed with Brine, dried over Na₂SO₄, filtered and concentrated under vacuum The residue was purified by FC on silica gel (eluting from 100% of DCM to DCM/MeOH 9:1) and then further purified by FC on NH column (eluting from cHex/AcOEt 8:2 to AcOEt 100%) affording the compound of formula N-[3-(azepan-1-yl)-4-(4-propylpiperazine-1-carbonyl)phenyl]cyclobutanecarboxamide.
Yield: 75 mg

1H NMR (500 MHz, DMSO-d6) δ 9.66 (s, 1 H), 7.33 (d, 1 H), 7.05 (dd, 1 H), 6.90 (d, 1 H), 3.24-3.17 (m, 5 H), 3.73-3.06 (m, 4 H), 2.25-2.13 (m, 5 H), 2.44-2.13 (m, 4 H), 2.09 (dtd, 2 H), 2.01-1.88 (m, 1 H), 1.85-1.75 (m, 1 H), 1.74-1.61 (m, 4 H), 1.60-1.47 (m, 4 H), 1.42 (sxt, 2 H), 0.84 (t, 3 H); LC-MS: m/z 427.32 (MH+).

Preparation: N-(3-(azepan-1-yl)-4-(4-propylpiperazine-1-carbonyl)phenyl)benzamide

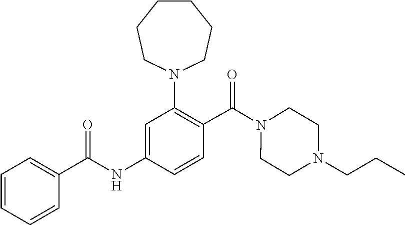 [SMILES: CCCN1CCN(C(=O)c2ccc(NC(=O)c3ccccc3)cc2N2CCCCCC2)CC1]

### Example 91

The synthesis of the title compound was effected analogously to the synthesis of the compound of Example 90 (N-(3-(azepan-1-yl )-4-(4-propylpiperazine-1-carbonyl)phenyl)cyclobutanecarboxamide) wherein benzoic acid was used instead of cyclobutanecarboxylic acid. The title compound was obtained in a yield of 66% (85 mg).

¹ NMR (400 MHz, DMSO-d₆) δ 10.18 (s, 1H), 7.99-7.91 (m, 2H), 7.63-7.45 (m, 4H), 7.31 (dd, 1H), 6.98 (d, 1H), 3.59 (d, 2H), 3.27-3.13 (m, 6H), 2.43-2.13 (m, 6H), 1.79-1.67 (m, 4H), 1.58 (dd, 4H), 1.49-1.39 (m, 2H), 0.86 (t, 3H); LC-MS: m/z 449.3 (MH+).

Preparation: N-(3-(azepan-1-yl-4-(4-propylpiperazine-1-carbonyl)phenyl)-2,2-difluorocyclopropane-1-carboxamide

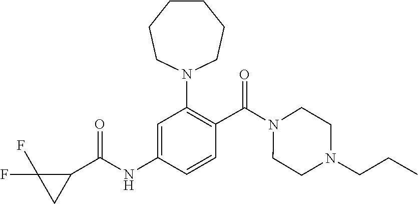 [SMILES: CCCN1CCN(C(=O)c2ccc(NC(=O)C3CC3(F)F)cc2N2CCCCCC2)CC1]

### Example 92

The synthesis of the title compound was effected analogously to the synthesis of the compound of Example 90 (N-(3-(azepan-1-yl)-4-(4-propylpiperazine-1-carbonyl)phenyl)cyclobutanecarboxamide) wherein 2,2-difluoro-1-cyclopropanecarboxylic acid was used instead of cyclobutanecarboxylic acid. The title compound was obtained in a yield of 45% (30 mg).

¹ NMR (400 MHz, DMSO-d₆) δ 10.37 (s, 1H), 7.30 (d, 1H), 7.07-6.91 (m, 2H), 3.74-3.48 (m, 2H), 3.27-3.14 (m, 6H), 2.93-2.72 (m, 1H), 2.43-2.17 (m, 6H), 2.06-1.92 (m, 2H), 1.74-1.66 (m, 4H), 1.63 —1.50 (m, 4H), 1.43 (q, 2H), 0.85 (t, 3H); LC-MS: m/z 449.6 (MH+).

Preparation: N-(3-(azepan-1-yl)-4-(4-propylpiperazine-1-carbonyl)phenyl)isobutyramide

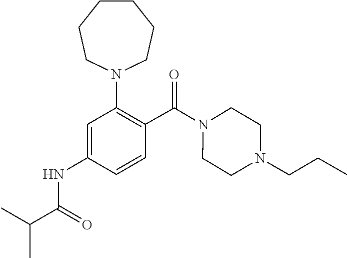 [SMILES: CCCN1CCN(C(=O)c2ccc(NC(=O)C(C)C)cc2N2CCCCCC2)CC1]

### Example 93

The synthesis of the title compound was effected analogously to the synthesis of the compound of Example 90 (N-(3-(azepan-1-yl)-4-(4-propylpiperazine-1-carbonyl)phenyl)cyclobutanecarboxamide) wherein 2-methylpropanoic acid was used instead of cyclobutanecarboxylic acid. The title compound was obtained in a yield of 63% (76 mg).

¹ NMR (400 MHz, DMSO-d₆) δ 9.77 (s, 1H), 7.36 (d, 1H), 7.05 (dd, 1H), 6.91 (d, 1H), 3.74-3.46 (m, 2H), 3.26-3.10 (m, 6H), 2.61— 2.55 (m, 1H), 2.45-2.10 (m, 6H), 1.76-1.63 (m, 4H), 1.61-1.52 (m, 4H), 1.43 (q, 2H), 1.09 (d, 6H), 0.85 (t, 3H); LC-MS: m/z 415.4 (MH+)

Preparation: N-(3-(azepan-1-yl)-4-(4-propylpiperazine-1-carbonyl)phenyl)propionamide

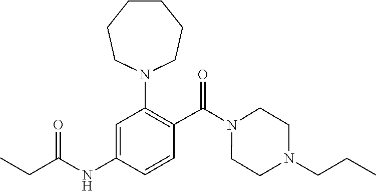 [SMILES: CCCN1CCN(C(=O)c2ccc(NC(=O)CC)cc2N2CCCCCC2)CC1]

### Example 94

A solution of [4-amino-2-(azepan-1-yl )phenyl]-(4-propylpiperazin-1-yl )methanone (50.0 mg, 0.150 mmol) and N,N-Diisopropylethylamine (0.05 mL, 0.290 mmol) in 1,4-Dioxane (1 mL) was cooled to 0° C. and after 10 min propanoyl chloride (0.02 mL, 0.220 mmol) was added. The reaction was warmed to RT and stirred at ON. The day after the reaction was diluted with DCM, washed with a s.s. of NaHCO₃, Brine, dried over Na₂SO₄, filtered and concentrated under vacuum. The residue was purified by FC on NH column (eluting from 100% of cHex to cHex/AcOEt 2:8) affording the product of formula N-[3-(azepan-1-yl)-4-(4-propylpiperazine-1-carbonyl)phenyl]propanamide.
Yield: 40.7 mg

¹ NMR (400 MHz, DMSO-d₆) δ 9.80 (s, 1H), 7.32 (d, 1H), 7.04 (dd, 1H), 6.91 (d, 1H), 3.79-3.42 (m, 2H), 3.24-3.11 (m, 6H), 2.43-2.14 (m, 8H), 1.68 (s, 4H), 1.60-1.53 (m, 4H), 1.43 (q, 2H), 1.08 (d, 3H), 0.85 (t, 3H); LC-MS: m/z 401.3 (MH+)

Preparation: N-(3-(azepan-1-yl)-4-(4-propylpiperazine-1-carbonyl)phenyl)acetamide

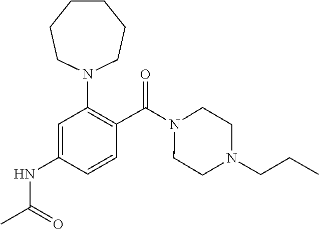 [SMILES: CCCN1CCN(C(=O)c2ccc(NC(C)=O)cc2N2CCCCCC2)CC1]

### Example 95

To a mixture of [4-amino-2-(azepan-1-yl)phenyI]-(4-propylpiperazin-1-yl)methanone (70.0 mg, 0.200 mmol) and N,N-Diisopropylethylamine (0.07 mL, 0.410 mmol) in DCM (2 mL) Acetic anhydride (0.02 mL, 0.200 mmol) was added and the reaction was stirred at RT ON. The day after the reaction was diluted with DCM, dried over Na₂SO₄, filtered and concentrated under vacuum. The residue was purified by FC on NH (eluting from cHex/AcOEt 8:2 to cHex/AcOEt 2:8) affording the product of formula N-[3-(azepan-1-yl)-4-(4-propylpiperazine-1-carbonyl)phenyl]acetamide.
Yield: 60 mg

¹ NMR (400 MHz, DMSO-d₆) 5 9.87 (s, 1H), 7.26 (d, 1H), 7.04 (dd, 1H), 6.91 (d, 1H), 3.73-3.46 (m, 2H), 3.25-3.04 (m, 6H), 2.44-2.13 (m, 6H), 2.02 (s, 3H), 1.75 -1.62 (m, 4H), 1.63 -1.51 (m, 4H), 1.43 (q, 2H), 0.85 (t, 3H); LC-MS: m/z 387.3 (MH+).

Preparation: [3-(azepan-1-yl)-4-(4-propylpiperazine-1-carbonyl)phenyl]urea

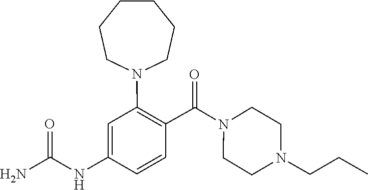 [SMILES: CCCN1CCN(C(=O)c2ccc(NC(N)=O)cc2N2CCCCCC2)CC1]

### Example 96

Potassium cyanate (111.95 mg, 1.38 mmol) was dissolved in warm Water (0.459 mL) and added gradually to a solution of [4-amino-2-(azepan-1-yl)phenyl]-(4-propylpiperazin-1-yl)methanone (50.0 mg, 0.140 mmol) in a mixture of Water (0.919 mL) and Acetic acid (0.100 mL). The reaction was stirred at RT for 1h. The reaction mixture was diluted with water and extracted with AcOEt 3 times. The organic phase was washed with Brine, dried over Na₂SO₄, filtered and concentrated under reduced pressure. The residue material was purified by FC on RP using acidic conditions (eluting from CH₃CN/H₂O 5:95 +0.1% of formic acid to CH₃CN/H₂O 95:5 +0.1% of formic acid). The factions containing the desired product were collected together and concentrated under vacuum. The resulting material was dissolved in DCM and washed with s. s. of NaHCO₃ and dried by filtration through a phase separator. The solvent was removed under vacuum affording the formula product [3-(azepan-1-yl)-4-(4-propylpiperazine-1-carbonyl)phenyl]urea
Yield: 6 mg

1H NMR (500 MHz, DMSO-d6) δ 8.54 (s, 1 H), 7.07 (s, 1 H), 6.91-6.77 (m, 2 H), 5.81 (s, 2 H), 3.18 (br t, 4 H), 3.72-3.03 (m, 4 H), 2.22 (br t, 2 H), 2.45-2.09 (m, 4 H), 1.76-1.47 (m, 8 H), 1.42 (sxt, 2 H), 0.84 (t, 3 H); LC-MS: m/z 388.6 (MH+).

Preparation: (2-(azepan-1-yl)-4-((5-cyclopropyl-4H-1,2,4-triazol-3-ypamino)phenyl)(4-propylpiperazin-1-yl)methanone

### Step a:

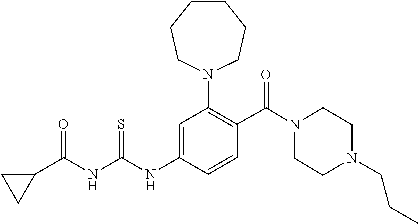 [SMILES: CCCN1CCN(C(=O)c2ccc(NC(=S)NC(=O)C3CC3)cc2N2CCCCCC2)CC1]

A mixture of potassium thiocyanate (193.75 mg, 1.99 mmol) and cyclopropanecarbonyl chloride (0.18 mL, 1.99 mmol) in MeCN (8 mL) was stirred at 80° C. for 2 h. After this time a solution of [4-amino-2-(azepan-1-yl)phenyl]-(4-propylpiperazin-1-yl)methanone (300.0 mg, 0.870 mmol) in MeCN (2 mL) was added and the reaction was stirred at the same temperature for 1 h. Then the reaction was cooled down to RT, filtered and the supernatant concentrated under vacuum. The residue was purified by FC on silica gel (eluting from 100% of DCM to DCM/MeOH 9:1) affording the intermediate 1[3-(azepan-1-yl)-4-(4-propylpiperazine-1-carbonyl)phenyl]-3-cyclopropanecarbonylthiourea
Yield: 316 mg
LC-MS: m/z 472.3 (MH+)

### Step b:

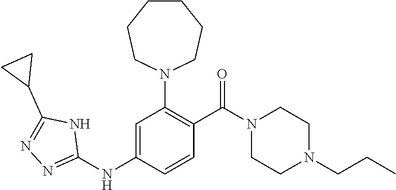 [SMILES: CCCN1CCN(C(=O)c2ccc(Nc3nnc(C4CC4)[nH]3)cc2N2CCCCCC2)CC1]

### Example 98

The intermediate 1[3-(azepan-1-yl)-4-(4-propylpiperazine-1-carbonyl)phenyl]-3-cyclopropanecarbonylthiourea (316.0 mg, 0.670 mmol) was dissolved in EtOH (12 mL), hydrazine;hydrate (0.7 mL, 9.21 mmol) was added and the reaction was stirred for 24h at RT. After this time the reaction was concentrated under vacuum and the residue was purified by FC on RP using basic conditions (eluent: 100% ammonium bicarbonate aqueous solution adjusted to pH 10 with ammonia to CH₃CN 100%) and it was further purified by FC on silica gel (eluting from 100% of DCM to DCM/MeOH 9:1) affording the formula compound 2-(azepan-1-yl )-4-((5-cyclopropyl-4H-1,2,4-triazol-3-ypamino)phenyl)(4-propylpiperazin-1-yl)methanone.
Yield: 13 mg

1H NMR (400 MHz, DMSO-d6) δ ppm 13.25-12.46 (m, 1 H), 9.31-8.76 (m, 1 H), 7.31 (s, 1 H), 6.97-6.73 (m, 2 H), 3.24-3.18 (m, 4 H), 3.79-3.02 (m, 4 H), 2.25-2.20 (m, 1 H), 2.43-2.10 (m, 4 H), 1.97-1.86 (m, 1 H), 1.80-1.48 (m, 8 H), 1.42 (sxt, 2 H), 0.98 (br d, 2 H), 0.89-0.78 (m, 5 H); LC-MS: m/z 452.34 (MH+).

Scheme 5, below, demonstrates synthesis of compounds in which X₂ is an imidazo[1,5-a]pyrazine group.

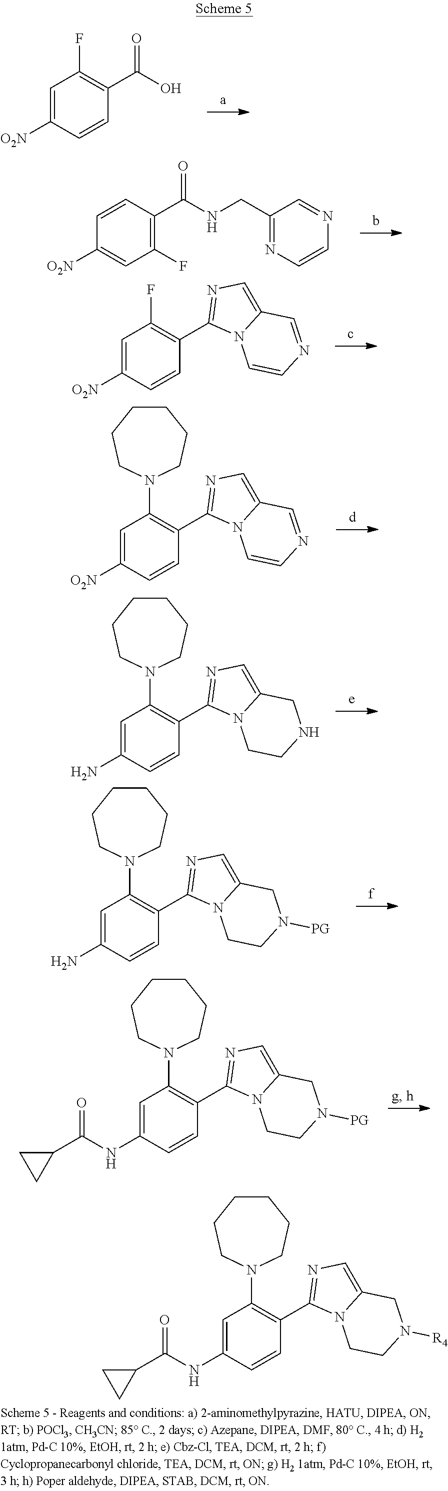

Preparation: 2-fluoro-4-nitro-N-[(pyrazin-2-yl)methyl]benzamide

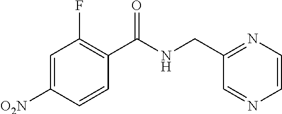 [SMILES: O=C(NCc1cnccn1)c1ccc([N+](=O)[O-])cc1F]

2-fluoro-4-nitrobenzoic acid (2.0 g, 10.8 mmol), HATU (4.93 g, 12.97 mmol)and N,N-Diisopropylethylamine (7.53 mL, 43.22 mmol) were mixed in DMF (50 mL) and stirred for 30 min, then 2-aminomethylpyrazine (1.41 g, 12.97 mmol) in DMF (5mL) at 0° C. was added. The reaction mixture was warmed to RT and stirred ON at the same temperature. The day after the reaction was concentrated to dryness and the residue was taken up with AcOEt and the organic phase was washed with s. s. of NaHCO₃, Brine, dried over Na₂SO₄, filtered and evaporated under vacuum. The residue was taken up with DCM and filtered. The solid was washed with DCM and dried under vacuum the product of formula 2-fluoro-4-nitro-N-(pyrazin-2-ylmethyl)benzamide which was used in the next step without further purification.
Yield: 1.9 g

1H NMR (400 MHz, DMSO-d6) δ 9.33 (s, 1H), 8.68 (d, 1H), 8.62 (dd, 1H), 8.57 (d, 1H), 8.24 (dd, 1H), 8.15 (dd, 1H), 7.92 (dd, 1H), 4.65 (d, 2H); LC-MS: m/z 277.04 (MH+).

Preparation: 3-(2-fluoro-4-nitrophenyl)imidazo[1,5-a]pyrazine

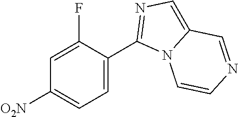 [SMILES: O=[N+]([O-])c1ccc(-c2ncc3cnccn23)c(F)c1]

2-fluoro-4-nitro-N-(pyrazin-2-ylmethyl)benzamide (1.9 g, 6.88 mmol) was dissolved in CH₃CN (95 mL), then Phosphorus(V) oxychloride (6.41 mL, 68.79 mmol) was added dropwise and the reaction mixture was stirred at 85° C. 48 h. The reaction mixture was cooled down to RT and evaporated. The residue was taken up with DCM and filtered. The solid was washed with DCM and dried under vacuum, affording the product of formula 3-(2-fluoro-4-nitrophenyl)imidazo[1,5-a]pyrazine.
Yield: 1 g 1H NMR (400 MHz, DMSO-d6) δ 9.29 (d, 1H), 8.40 (dd, 1H), 8.29 (dd, 1H), 8.24-8.18 (m, 2H), 8.11 (dd, 1H), 7.74 (d, 1H); LC-MS: m/z 259.02 (MH+).
   Preparation: 3-[2-(azepan-1-yl)-4-nitrophenyl]imidazo[1,5-a]pyrazine

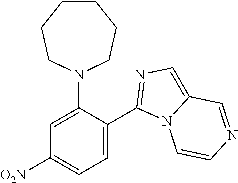 [SMILES: O=[N+]([O-])c1ccc(-c2ncc3cnccn23)c(N2CCCCCC2)c1]

To a solution of N,N-Diisopropylethylamine (0.62 mL, 3.56 mmol) and Homopiperidine (1.2 mL, 10.69 mmol) in DMF (9 mL), 3-(2-fluoro-4-nitrophenyl)imidazo[1,5-a]pyrazine (920.0 mg, 3.56 mmol) was added and the reaction was stirred at 80 C for 4 h. After this time the reaction was cooled down to RT and diluted with H₂O and the mixture was extracted with AcOEt 3 times. The combined organic fractions were washed with Brine, dried over Na₂SO₄, filtered and concentrated under vacuum. The residue was purified by FC NH column (eluting from cHex to cHex/AcOEt 1:1). The fractions contain desired product were combined and evaporated. The residue was triturated with pentane affording the product of formula 3-[2-(azepan-1-yl)-4-nitrophenyl]imidazo[1,5-a]pyrazine.
Yield: 800 mg

¹ NMR (400 MHz, Chloroform-d) δ 9.09 (d, 1H), 8.02 (dd, 2H), 7.84 (dd, 1H), 7.70 (d, 1H), 7.61 (d, 1H), 7.35 (ddd, 1H), 3.01 (s, 4H), 1.45 (s, 9H); LC-MS: m/z 338.2 (MH+).

Preparation: 3-(4-nitro-2-pyrrolidin-1-ylphenyl)imidazo[1,5-a]pyrazine

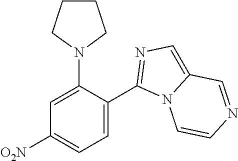 [SMILES: O=[N+]([O-])c1ccc(-c2ncc3cnccn23)c(N2CCCC2)c1]

The synthesis of the title compound was effected analogously to the synthesis of 3-[2-(azepan-1-yl)-4-nitrophenyl]imidazo[1,5-a]pyrazine wherein pyrrolidine was used instead of Homopiperidine. The title compound was obtained in a yield of 99% (600 mg).

1H NMR (400 MHz, DMSO-d6) δ 9.17 (d, 1H), 8.06 (d, 1H), 7.72-7.55 (m, 5H), 2.72-2.62 (m, 4H), 1.73-1.60 (m, 4H); LC-MS: 310.12 (MH+).

Preparation: Syn/Anti 3-[2-(3,5-dimethylpiperidin-1-yl)-4-nitrophenyl]imidazo[1,5-a]pyrazine

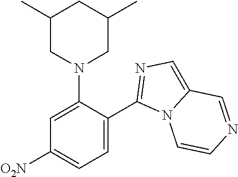 [SMILES: CC1CC(C)CN(c2cc([N+](=O)[O-])ccc2-c2ncc3cnccn23)C1]

The synthesis of the title compound was effected analogously to the synthesis of 3-[2-(azepan-1-yl)-4-nitrophenyl]imidazo[1,5-a]pyrazine wherein 3,5-dimethylpiperidine was used instead of Homopiperidine. The title compound was obtained in a yield of 87% (475 mg) as a mixture of steroisomers.
LC-MS: m/z 352.15 (MH+)
   Preparation: 3-(azepan-1-yl)-4-(5,6,7,8-tetrahydroimidazo[1,5-a]pyrazin-3-yl)aniline

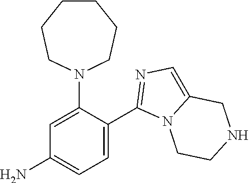 [SMILES: Nc1ccc(-c2ncc3n2CCNC3)c(N2CCCCCC2)c1]

To a mixture of 3-[2-(azepan-1-yl)-4-nitrophenyl]imidazo[1,5-a]pyrazine (380.0 mg, 1.13 mmol) and acetic acid (67.64 mg, 1.13 mmol) in MeOH (10 mL) 20% palladium(2+) hydroxide (395.43 mg, 0.560 mmol) was added and the reaction was stirred under H₂ atmosphere at RT for 2 hrs. The reaction mixture was then filtered through celite and the solvent removed under vacuum. The residue was purified by SCX first washing with MeOH and then with NH₃ 1M in MeOH affording the product of formula 3-(azepan-1-yl)-4-(5,6,7,8-tetrahydroimidazo[1,5-a]pyrazin-3-ypaniline.
Yield: 300 mg

¹ NMR (400 MHz, Chloroform-d) δ 7.10 (d, 1H), 6.87-6.84 (m, 1H), 6.36 (d, 1H), 6.28 (dd, 1H), 3.83-3.63 (m, 4H), 3.15 (t, 2H), 3.11-3.04 (m, 4H), 1.58-1.43 (m, 9H); LC-MS: m/z 312.2 (MH+).

Preparation: 3-pyrrolidin-1-yl-4-(5,6,7,8-tetrahydroimidazo[1,5-a]pyrazin-3-yl)aniline

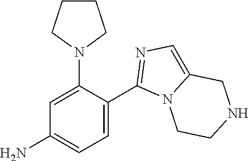 [SMILES: Nc1ccc(-c2ncc3n2CCNC3)c(N2CCCC2)c1]

A mixture of 3-( 4-nitro-2-pyrrolidin-1-ylphenyl)imidazo[1,5-a]pyrazine (597.0 mg, 1.93 mmol), acetic acid (0.33 mL, 5.79 mmol) and 20% palladium(2+) hydroxide (271.04 mg, 0.390 mmol) in Methanol (15 mL) was stirred under H2 atmosphere at RT for 1 h. After this time the mixture was filtered through a pad of celite and the solvent removed under vacuum. The residue was purified by SCX cartridge first washing with MeOH and then eluting with NH₃ 1M in MeOH, affording the product of formula 3-pyrrolidin-1-yl-4-(5,6,7,8-tetrahydroimidazo[1,5-a]pyrazin-3-ypaniline.
Yield: 358 mg

¹ NMR (400 MHz, DMSO-d₆) δ 6.81-6.50 (m, 2H), 6.11-5.86 (m, 1H), 5.02 (d, 1H), 3.96-3.78 (m, 2H), 3.63-3.41 (m, 2H), 3.31— 3.21 (m, 2H), 2.98-2.83 (m, OH), 2.81-2.65 (m, 4H), 1.70 (q, 4H), LC-MS: 284.0

Preparation: Syn/Anti 3-(3,5-dimethylpiperidin-1-yl)-4-(5,6,7,8-tetrahydroimidazo[1,5-a]pyrazin ypaniline

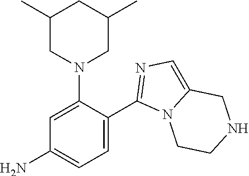 [SMILES: CC1CC(C)CN(c2cc(N)ccc2-c2ncc3n2CCNC3)C1]

The synthesis of the title compound was effected analogously to the synthesis of 3-pyrrolidin-1-yl-4-(5,6,7,8-tetrahydroimidazo[1,5-a]pyrazin-3-ypaniline wherein Syn/Anti 3-[2-(3,5-dimethylpiperidin-1-yl)-4-nitrophenyl]imidazo[1,5-a]pyrazine was used instead of 3-[4-nitro-2-pyrrolidin-1-ylphenyl)imidazo[1,5-a]pyrazine. The title compound was obtained in a yield of 47% (120 mg) as a mixture of steroisomers.
LC-MS: m/z 326.1 (MH+)
   Preparation: benzyl 3-[4-amino-2-(azepan-1-yl)phenyl]-6,8-dihydro-5H-imidazo[1,5-a]pyrazine-7-carboxylate

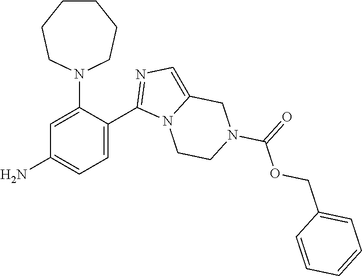 [SMILES: Nc1ccc(-c2ncc3n2CCN(C(=O)OCc2ccccc2)C3)c(N2CCCCCC2)c1]

A solution of 3-(azepan-1-yl)-4-(5,6,7,8-tetrahydroimidazo[1,5-a]pyrazin-3-ypaniline (300.0 mg, 0.960 mmol) and triethylamine (0.27 mL, 1.93 mmol) in DCM (16 mL) was cooled to 0 C and after 5 minutes Cbz-CI (0.12 mL, 0.870 mmol) was added dropwise. The reaction was stirred at same temperature for 2 hrs. HCI 1N was added and the organic phase was separated. Then the aqueous phase was brought until pH 8 with NaHCO₃ s.s. and extracted with DCM 3 times. The organic phase was dried over Na₂SO₄, filtered and evaporated. The residue was purified by FC on NH column (eluting from 100% of AcOEt to AcOEt/MeOH 9:1) affording the product of formula benzyl 3-[4-amino-2-(azepan-1-yl)phenyl]-6,8-dihydro-5H-imidazo[1,5-a]pyrazine-7-carboxylate.
Yield: 165 mg

¹ NMR (400 MHz, Chloroform-d) δ 7.42-7.35 (m, 5H), 7.12 (d, 1H), 6.92 (s, 1H), 6.36 (d, 1H), 6.29 (dd, 1H), 5.21 (s, 2H), 4.81-4.69 (m, 2H), 3.86-3.68 (m, 4H), 3.03 (t, 4H), 1.58-1.37 (m, 8H); LC-MS: m/z 446.3 (MH+).

Preparation: benzyl 3-(4-amino-2-pyrrolidin-1-ylphenyI)-6,8-dihydro-5H-imidazo[1,5-a]pyrazine-7-carboxylate

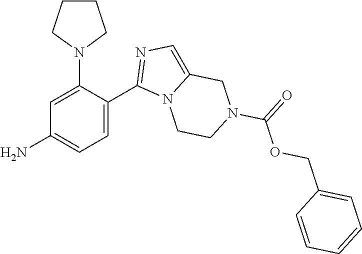 [SMILES: Nc1ccc(-c2ncc3n2CCN(C(=O)OCc2ccccc2)C3)c(N2CCCC2)c1]

The synthesis of the title compound was effected analogously to the synthesis of benzyl 3-[4-amino-2-(azepan-1-yl)phenyI]-6,8-dihydro-5H-imidazo[1,5-a]pyrazine-7-carboxylate wherein 3-pyrrolidin-1-yl-4-(5,6,7,8-tetrahydroimidazo[1,5-a]pyrazin-3-yl)aniline was used instead of 3-(azepan-1-yl)-4-(5,6,7,8-tetrahydroimidazo[1,5-a]pyrazin-3-yl)aniline. The title compound was obtained in a yield of 43% (126 mg).

1H NMR (400 MHz, DMSO-d6) δ 7.43-7.26 (m, 5H), 6.81— 6.73 (m, 2H),6.07-5.98 (m, 2H), 5.13 (s, 2H), 5.09 (s, 2H), 4.73-4.55 (m, 2H), 3.78-3.56 (m, 4H), 2.76-2.59 (m, 4H), 1.75-1.59 (m, 4H); LC-MS: m/z 418.2 (MH+).

Preparation: benzyl 3[2-(azepan-1-yl)-4-(cyclopropanecarbonylamino)phenyl]-6,8-dihydro-5H-imidazo[1,5-a]pyrazine-7-carboxylate

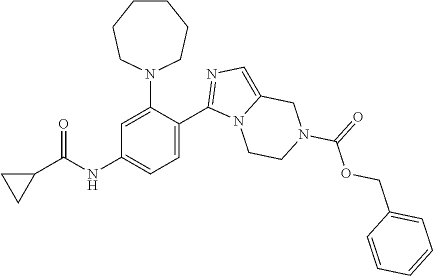 [SMILES: O=C(Nc1ccc(-c2ncc3n2CCN(C(=O)OCc2ccccc2)C3)c(N2CCCCCC2)c1)C1CC1]

### Example 99

A solution of benzyl 3-[4-amino-2-(azepan-1-yl )phenyl]-6,8-dihydro-5H-imidazo[1,5-a]pyrazine carboxylate (165.0 mg, 0.370 mmol) and N,N-Diisopropylethylamine (0.13 mL, 0.740 mmol) in DCM (3 mL) was cooled to 0 C and after 5 minutes cyclopropanecarbonyl chloride (0.05 mL, 0.560 mmol) was added dropwise. The reaction was stirred at RT ON. The day after the reaction was diluted with DCM and washed with a s.s. of NaHCO₃ and Brine. The organic phase was dried over Na₂SO₄, filtered and concentrated under vacuum. The residue was purified by FC on NH column (eluting from cHex/AcOEt 95:5 to cHex/AcOEt 40:60) affording the product of formula benzyl 3[2-(azepan-1-yl)-4-(cyclopropanecarbonylamino)phenyl]-6,8-dihydro-5H-imidazo[1,5-a]pyrazine-7-carboxylate.
Yield: 175 mg LC-MS: m/z 514.3 (MH+)
   Preparation: benzyl 3-[4-(cyclopropanecarbonylamino)-2-pyrrolidin-1-ylphenyl]-6,8-dihydro-5H-imidazo[1,5-a]pyrazine-7-carboxylate

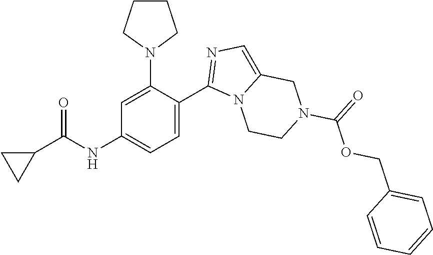 [SMILES: O=C(Nc1ccc(-c2ncc3n2CCN(C(=O)OCc2ccccc2)C3)c(N2CCCC2)c1)C1CC1]

### Example 100

The synthesis of the title compound was effected analogously to the synthesis of the compound of Example 99 (benzyl 3-[2-(azepan-1-yl)-4-(cyclopropanecarbonylamino)phenyl]-6,8-dihydro-5H-imidazo[1,5-a]pyrazine-7-carboxylate) wherein benzyl 3-(4-amino-2-pyrrolidin-1-ylphenyI)-6,8-dihydro-5H-imidazo[1,5-a]pyrazine-7-carboxylate was used instead of benzyl 3-[4-amino-2-(azepan-1-yl)phenyl]-6,8-dihydro-5H-imidazo[1,5-a]pyrazine-7-carboxylate. The title compound was obtained in a yield of 88% (130 mg).

1H NMR (400 MHz, DMSO-d6) δ 10.13 (s, 1H), 7.44-7.26 (m, 5H), 7.19 (d, 1H), 7.07-6.93 (m, 2H), 6.82 (s, 1H), 5.13 (s, 2H), 4.67 (s, 2H), 3.68 (s, 4H), 2.73 (s, 4H), 1.84— 1.62 (m, 5H), 0.84-0.71 (m, 4H); LC-MS: m/z 486.2 (MH+).

Preparation: Syn/Ant benzyl 3-[4-(cyclopropanecarbonylamino)-2-(3,5-dimethylpiperidin-1-yl )phenyl]-6,8-dihydro-5H-imidazo[1,5-a]pyrazine-7-carboxylate

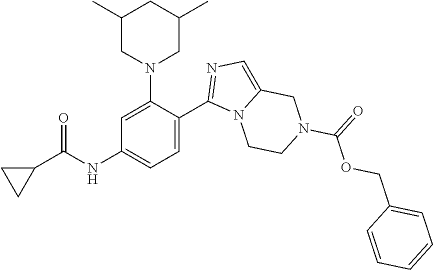 [SMILES: CC1CC(C)CN(c2cc(NC(=O)C3CC3)ccc2-c2ncc3n2CCN(C(=O)OCc2ccccc2)C3)C1]

### Example 101

A solution of 3-(3,5-dimethylpiperidin-1-yl )-4-(5,6,7,8-tetrahydroimidazo[1,5-a]pyrazin-3-yl)aniline (119.0 mg, 0.370 mmol) and triethylamine (0.1 mL, 0.730 mmol) in DCM (7 mL) was cooled to 0° C. and after 10 minutes carbonochloridic acid (phenylmethyl) ester (0.05 mL, 0.330 mmol) was added dropwise. The reaction was stirred at the same temperature for 2 h. After this time further triethyla mine (0.1 mL, 0.730 mmol) was added followed by cyclopropanecarbonyl chloride (0.04 mL, 0.400 mmol). The reaction was stirred at RT/ON. The day after the mixture reaction was diluted with DCM and the organic phase was washed with s. s. of NaHCO₃, Brine, dried over a phase separator and concentrated under vacuum. The residue was purified by FC on silica gel (eluting from 100% of cHex to 100% of AcOEt) affording the product of formula benzyl 3-[4-(cyclopropanecarbonylamino)-2-(3,5-dimethylpiperidin-1-yl)phenyl]-6,8-dihydro-5H-imidazo[1,5-a]pyrazine-7-carboxylate as a mixture of Syn/Anti isomers.
Yield: 121 mg
LC-MS: 528.23 (MH+)
   Preparation: N-[3-(azepan-1-yl)-4-(5,6,7,8-tetrahydroimidazo[1,5-a]pyrazin-3-yl)phenyl]cyclopropanecarboxamide

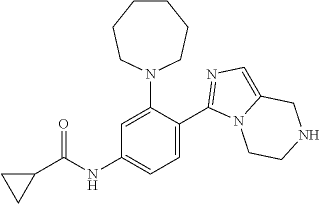 [SMILES: O=C(Nc1ccc(-c2ncc3n2CCNC3)c(N2CCCCCC2)c1)C1CC1]

To a mixture of benzyl 3[2-(azepan-1-yl)-4-(cyclopropanecarbonylamino)phenyl]-6,8-dihydro-5H-imidazo[1,5-a]pyrazine-7-carboxylate (175.0 mg, 0.340 mmol) and acetic acid (0.35 mL, 6.13 mmol) in Ethanol (7 mL), 10% palladium on carbon (36.26 mg, 0.030 mmol) was added and the reaction mixture was stirred under H2 atmosphere at RT for 3 h. Then the reaction was filtered through a pad of celite and concentrated under vacuum. The residue was purified by SCX first washing with MeOH and then with NH₃ 1M in MeOH affording the product of formula N-[3-(azepan-1-yl)-4-(5,6,7,8-tetrahydroimidazo[1,5-a]pyrazin-3-yl)phenyl]cyclopropanecarboxamide.
Yield: 95 mg
LC-MS: m/z 380.3 (MH+).
   Preparation: N-[3-pyrrolidin-1-yl-4-(5,6,7,8-tetrahydroimidazo[1,5-a]pyrazin-3-yl)phenyl]cyclopropanecarboxamide

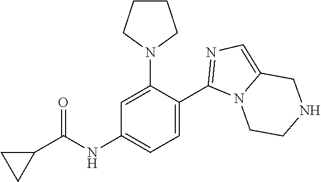 [SMILES: O=C(Nc1ccc(-c2ncc3n2CCNC3)c(N2CCCC2)c1)C1CC1]

The synthesis of the title compound was effected analogously to the synthesis of N-[3-(azepan-1-yl)-4-(5,6,7,8-tetrahydroimidazo[1,5-a]pyrazin-3-yl)phenyl] Cyclopropanecarboxamide wherein benzyl 3-[4-(cyclopropanecarbonylamino)-2-pyrrolidin-1-ylphenyI]-6,8-dihydro-5H-imidazo[1,5-a]pyrazine-7-carboxylate was used instead of benzyl 3[2-(azepan-1-yl)-4-(cyclopropanecarbonylamino)phenyl]-6,8-dihydro-5H-imidazo[1,5-a]pyrazine-7-carboxylate. The title compound was obtained in a yield of 87% (81 mg).

1H NMR (400 MHz, DMSO-d6) δ 10.11 (s, 1H), 7.15 (s, 1H), 6.97 (s, 2H), 6.65 (d, 1H), 3.88 (s, 2H), 3.47 (s, 2H), 2.92 (t, 2H), 2.75 (s, 4H), 1.84-1.65 (m, 5H), 0.85-0.71 (m, 4H); LC-MS: 352.1 (MH+).

Preparation: Syn/Anti N-[3-(3,5-dimethylpiperidin-1-yl)-4-(5,6,7,8-tetrahydroimidazo[1,5-a]pyrazin-3-yl)phenyl]cyclopropanecarboxamide

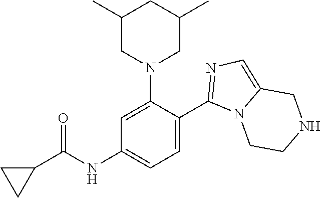 [SMILES: CC1CC(C)CN(c2cc(NC(=O)C3CC3)ccc2-c2ncc3n2CCNC3)C1]

The synthesis of the title compound was effected analogously to the synthesis of N-[3-(azepan-1-yl)-4-(5,6,7,8-tetrahydroimidazo[1,5-a]pyrazin-3-yl)phenyl] Cyclopropanecarboxamide wherein Syn/Anti benzyl 3-[4-(cyclopropanecarbonylamino)-2-(3,5-dimethylpiperidin-1-yl)phenyl]-6,8-dihydro-5H-imidazo[1,5-a]pyrazine-7-carboxylate was used instead of benzyl 3[2-(azepan-1-yl)-4-(cyclopropanecarbonylamino)phenyl]-6,8-dihydro-5H-imidazo[1,5-a]pyrazine-7-carboxylate. The title compound was obtained in a yield of 80% (72 mg) as a mixture of Syn/Anti isomers.
LC-MS: m/z 394.26 (MH+)
   Preparation: N-[3-(azepan-1-yl)-4-{7-methyl-5H,6H,7H,8H-imidazo[1,5-a]pyrazin-3-yl}phenyl]cyclopropanecarboxamide

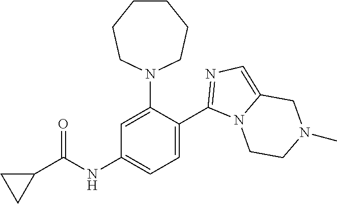 [SMILES: CN1CCn2c(cnc2-c2ccc(NC(=O)C3CC3)cc2N2CCCCCC2)C1]

### Example 102

To a mixture of N-[3-(azepan-1-yl)-4-(5,6,7,8-tetrahydroimidazo[1,5-a]pyrazin-3-yl)phenyl]cyclopropanecarboxamide (45.0 mg, 0.120 mmol) and 37% formaldehyde in H2O (0.09 mL, 1.19 mmol) in Methanol (2 mL), sodium triacetoxyborohydride (150.79 mg, 0.710 mmol) was added at 0° C. The reaction was warmed to RT and stirred ON. The solvent was evaporated, AcOEt and Water were added and the organic phase was separated and dried. The aqueous phase was back-extracted twice with AcOEt, then the combined organic fractions were washed with Brine, dried over Na₂SO₄, filtered and concentrated under vacuum. The residue was purified by FC on NH column (eluting from Cy/EtOAc from 100:0 to 10:90) affording the product of formula N-[3-(azepan-1-yl)-4-(7-methyl-6,8-dihydro-5H-imidazo[1,5-a]pyrazin-3-yl)phenyl]cyclopropanecarboxamide.
Yield: 21.5 mg

¹ NMR (400 MHz, DMSO-d₆) δ 10.17 (s, 1H), 7.47 (d, 1H), 7.08 (dd, 1H), 7.03 (d, 1H), 6.70 (d, 1H), 3.64 (s, 2H), 3.54 (s, 2H), 2.98 (t, 4H), 2.65 (t, 2H), 2.35 (s, 3H), 1.78 (qd, 1H), 1.46 (s, 8H), 0.87-0.74 (m, 4H); LC-MS: m/z 394.3 (MH+).

Preparation: N-[4-(7-methyl-6,8-dihydro-5H-imidazo[1,5-a]pyrazin-3-yl)-3-pyrrolidin-1-ylphenyl]cyclopropanecarboxamide

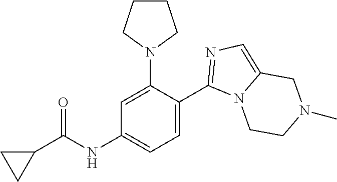 [SMILES: CN1CCn2c(cnc2-c2ccc(NC(=O)C3CC3)cc2N2CCCC2)C1]

### Example 103

The synthesis of the title compound was effected analogously to the synthesis of the compound of Example 102 (N-[3-(azepan-1-yl)-4-{7-methyl-5H,6H,7H,8H-imidazo[1,5-a]pyrazin-3-yl}phenyl]cyclopropanecarboxamide) wherein N-[3-pyrrolidin-1-yl-4-(5,6,7,8-tetrahydroimidazo[1,5-a]pyrazin-3-yl)phenyl]cyclopropanecarboxamide was used instead of N-[3-(azepan-1-yl)-4-(5,6,7,8-tetrahydroimidazo[1,5-a]pyrazin-3-yl)phenyl]cyclopropanecarboxamide. The title compound was obtained in a yield of 64% (54 mg).

1H NMR (400 MHz, DMSO-d6) δ 10.12 (br s, 1 H), 7.22-7.13 (m, 1 H), 7.05-6.94 (m, 2 H), 6.69 (s, 1 H), 3.65-3.56 (m, 2 H), 3.53 (s, 2 H), 2.79-2.70 (m, 4 H), 2.64 (br t, 2 H), 2.34 (s, 3 H), 1.83-1.76 (m, 1 H), 1.75-1.69 (m, 4 H), 0.84-0.73 (m, 4 H); LC-MS: m/z 355.2 (MH+).

Preparation: Syn/Anti N-[3-(3,5-dimethylpiperidin-1-yl)-4-(7-methyl-6,8-dihydro-5H-imidazo[1,5-a]pyrazin-3-yl)phenyl] cyclopropanecarboxamide

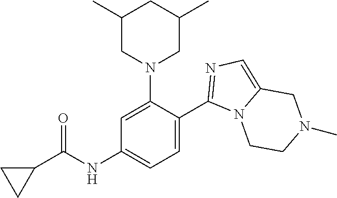 [SMILES: CC1CC(C)CN(c2cc(NC(=O)C3CC3)ccc2-c2ncc3n2CCN(C)C3)C1]

### Example 104a, 104b

The synthesis of the title compound was effected analogously to the synthesis of the compound of Example 102 (N-[3-(azepan-1-yl)-4-{7-methyl-5H,6H,7H,8H-imidazo[1,5-a]pyrazin-3-yl}phenyl]cyclopropanecarboxamide) wherein Syn/Anti N-[3-(3,5-dimethylpiperidin-1-yl)-4-(5,6,7,8-tetrahydroimidazo[1,5-a]pyrazin-3-yl)phenyl]cyclopropanecarboxamide was used instead of N-[3-(azepan-1-yl)-4-(5,6,7,8-tetrahydroimidazo[1,5-a]pyrazin-3-yl)phenyl]cyclopropanecarboxamide. The title compound was obtained in a yield of 99% (75 mg) as a mixture of Syn/Anti isomers.
LC-MS: m/z 409.9 (MH+).
   The mixture of isomers was then separated into isomers by Semi preparative MDAP method

| | | |
|---|---|---|
| | Column: | XSelect CSH Prep. C18 5 μm OBD 30 × 100 mm @ room T |
| | Solvents: | A = H₂O + 0.1% HCOOH B = Acetonitrile |
| | Method: | from 17% B to 99.9% B in 15 min |
| Preparative achiral | Injection loop: | 1 mL (each injection) |
| chromatography | Flow rate (ml/min): | 40 ml/min |
| protocol: | UV detection range: | 210 nm to 350 nm |

| | |
|---|---|
| 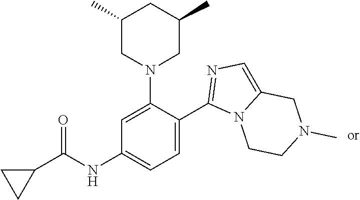 [SMILES: C[C@@H]1C[C@@H](C)CN(c2cc(NC(=O)C3CC3)ccc2-c2ncc3n2CCN(C)C3)C1] | First eluted compound, Isomer 1: Anti N-[4-(4-methyl-2-phenylpiperazine-1- carbonyl)-3-(3-methylpyrazol-1-yl)phenyl] cyclopropanecarboxamide Yield: 7% (5 mg) 1H NMR (500 MHz, DMSO-d6 ) δ ppm 10.26 (s, 1 H), 7.48 (d, 1 H), 7.26 (dd, 1 H), 7.07 (d, 1 H), 6.72 (s, 1 H), 3.91-3.75 (m, 1 H), 3.62-3.41 (m, 3 H), 2.72-2.62 (m, 4 H), 2.48-2.42 (m, 2 H), 2.33 (s, 3 H), 1.83-1.67 (m, 3 H), 1.28-1.21 (m, 2 H), 0.85-0.76 (m, 4 H), 0.71 (d, 6 H), LC-MS: m/z 409.9 (MH+) Example 104a |
| 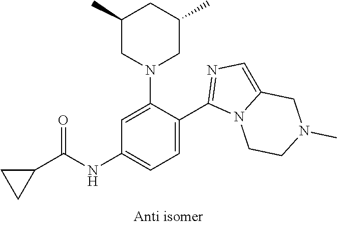 [SMILES: C[C@H]1C[C@H](C)CN(c2cc(NC(=O)C3CC3)ccc2-c2ncc3n2CCN(C)C3)C1] | |
| 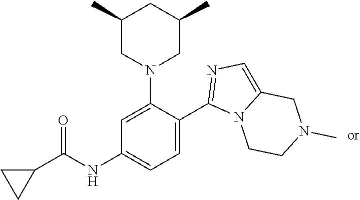 [SMILES: C[C@@H]1C[C@H](C)CN(c2cc(NC(=O)C3CC3)ccc2-c2ncc3n2CCN(C)C3)C1] | Second eluted compound, Isomer 2: Syn N-[4-(4-methyl-2-phenylpiperazine-1- carbonyl)-3-(3-methylpyrazol-1-yl)phenyl] cyclopropanecarboxamide Yield: 58% (43.4 mg) 1H NMR (500 MHz, DMSO-d6 ) δ ppm 10.24 (s, 1 H), 7.40 (d, 1 H), 7.23 (dd, 1 H), 7.13 (d, 1 H), 6.72 (s, 1 H), 3.86-3.70 (m, 2 H), 3.52 (s, 2 H), 2.94 (br d, 2 H), 2.67- 2.60 (m, 2 H), 2.34 (s, 3 H), 2.04-1.86 (m, 2 H), 1.81-1.75 (m, 1 H), 1.68 (br d, 1 H), 1.49-1.37 (m, 2 H), 0.82-0.77 (m, 4 H), 0.73 (br d, 6 H), 0.51 (q, J = 12.1 Hz, 1 H), LC-MS: m/z 409.9 (MH+) Example 104b |
| 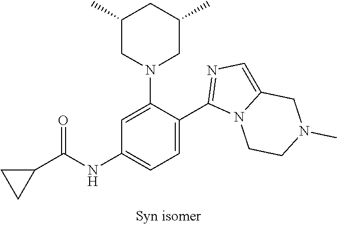 [SMILES: C[C@@H]1C[C@H](C)CN(c2cc(NC(=O)C3CC3)ccc2-c2ncc3n2CCN(C)C3)C1] | |

Preparation: N-[3-(azepan-1-yl)-4-(7-propyl-6,8-dihydro-5H-imidazo[1,5-a]pyrazin-3-yl)phenyl]cyclopropanecarboxamide

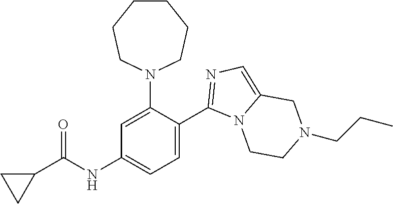 [SMILES: CCCN1CCn2c(cnc2-c2ccc(NC(=O)C3CC3)cc2N2CCCCCC2)C1]

### Example 105

To a mixture of N-[3-(azepan-1-yl)-4-(5,6,7,8-tetrahydroimidazo[1,5-a]pyrazin-3-yl)phenyl]cyclopropanecarboxamide (35.0 mg, 0.090 mmol) and propanal (6.43 mg, 0.110 mmol) in MeCN (2 mL), sodium triacetoxyborohydride (39.09 mg, 0.180 mmol) was added at 0° C. The reaction was warmed to RT and stirred ON. The mixture was directly purified by SCX first washing with MeOH and then with NH₃ 1M in MeOH and further purified by FC on NH column (eluting from 100% of cHex to cHex/AcOEt 1:1) affording the product of formula N-[3-(azepan-1-yl)-4-(7-propyl-6,8-dihydro-5H-imidazo[1,5-a]pyrazin-3-yl)phenyl]cyclopropanecarboxamide.
Yield: 15 mg

¹ NMR (400 MHz, DMSO-d₆) δ 10.17 (s, 1H), 7.48 (d, 1H), 7.09 (dd, 1H), 7.03 (d, 1H), 6.71 (d, 1H), 3.69-3.56 (m, 4H), 2.98 (t, 4H), 2.71 (t, 2H), 2.45-2.40 (m, 2H), 1.82-1.75 (m, 1H), 1.56-1.37 (m, 10H), 0.89 (t, 3H), 0.83-0.77 (m, 4H); LC-MS: m/z 422.0 (MH+).

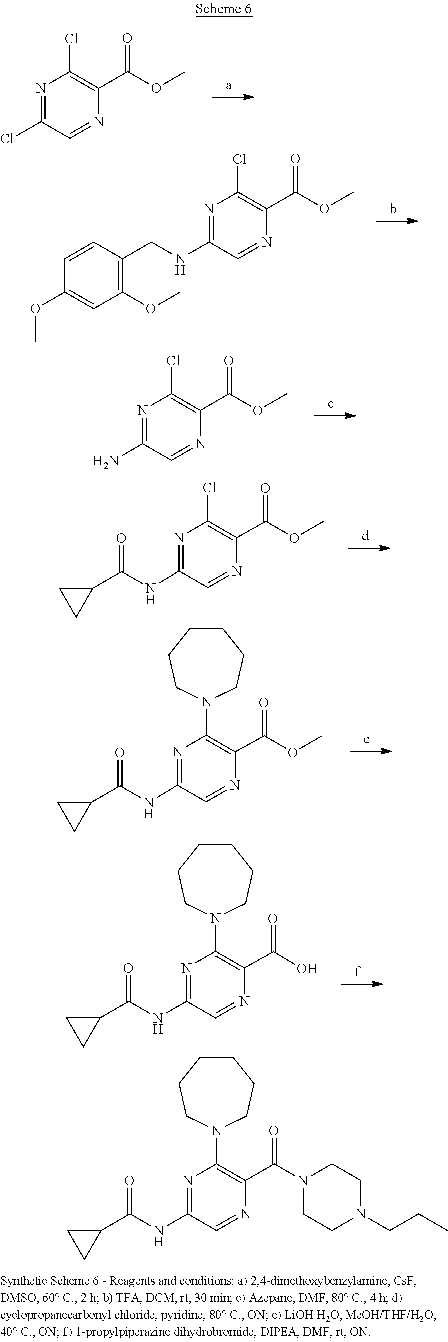

Preparation: methyl 3-chloro-5-{[(2,4-dimethoxyphenyl)methyl]amino}pyrazine-2-carboxylate

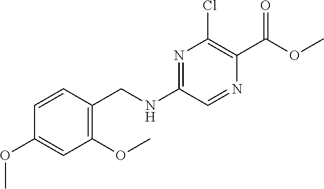 [SMILES: COC(=O)c1ncc(NCc2ccc(OC)cc2OC)nc1Cl]

To a solution of methyl 3,5-dichloropyrazine-2-carboxylate (1.0 g, 4.83 mmol) in DMSO (16.1 mL) (2,4-dimethoxyphenyl)methanamine (0.73 mL, 4.83 mmol) and cesiumfluoride (0.73 g, 4.83 mmol) were added. The reaction was stirred at 60° C. for 2 h. After this time the reaction was cooled down to rt and diluted with H₂O. The reaction was extracted with AcOEt 3 times. Then the organic phase was washed with Brine, dried over Na₂SO₄, filtered and concentrated under vacuum. The residue was purified by FC on NH column (from 100% of cHex to cHex/AcOEt 1:1) affording methyl the product of formula 3-chloro-5-[(2,4-dimethoxyphenyl)methylamino]pyrazine-2-carboxylate.
Yield: 1.4 g

¹ NMR (400 MHz, DMSO-d₆) δ 8.47 (s, 1H), 7.95 (s, 1H), 7.18 (d, 1H), 6.59 (d, 1H), 6.50 (dd, 1H), 4.39 (d, 2H), 3.81 (s, 3H), 3.79 (s, 3H), 2.55 (s, 3H); LC-MS: m/z 338.3 (MH+).

Preparation: methyl 5-amino-3-chloropyrazine-2-carboxylate

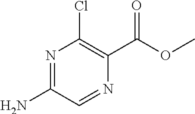

A mixture of methyl 3-chloro-5-[(2,4-dimethoxyphenyμmethylamino]pyrazine-2-carboxylate (400.0 mg, 1.18 mmol) and Trifluoroacetic acid (0.95 mL, 12.39 mmol) was stirred at rt for 30 min. After this time the reaction was concentrated under vacuum. The residue was directly purified by SCX first washing with MeOH and then with NH₃ 1M in MeOH affording the product of formula methyl 5-amino-3-chloropyrazine-2-carboxylate, which was used in the next step without further purification.
Yield: 220 mg

¹ NMR (400 MHz, DMSO-d₆) δ 7.85 (s, 1H), 7.69 (s, 2H), 3.79 (s, 3H); LC-MS: m/z 186.11 (M-H)

Preparation: methyl 3-chloro-5-(cyclopropanecarbonylamino)pyrazine-2-carboxylate

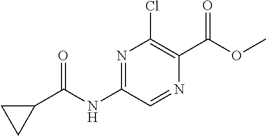 [SMILES: COC(=O)c1ncc(NC(=O)C2CC2)nc1Cl]

To a suspension of methyl 5-amino-3-chloropyrazine-2-carboxylate (220.0 mg, 1.17 mmol) in Pyridine (6 mL) cyclopropanecarbonyl chloride (0.32 mL, 3.52 mmol) was added and the reaction was stirred at 80 ° C. ON. The day after the reaction was concentrated under vacuum. The residue was dissolved in AcOEt and washed with a s.s. of NaHCO₃, Brine, dried over Na₂SO₄, filtered and concentrated under vacuum affording the product of formula methyl 3-chloro-5-(cyclopropanecarbonylamino)pyrazine-2-carboxylate. The product was used in the next step without further purification.
Yield: 220 mg

¹ NMR (400 MHz, DMSO-d₆) δ 11.75 (s, 1H), 9.32 (s, 1H), 3.90 (s, 3H), 2.04 (tt, 1H), 0.95-0.88 (m, 4H).

Preparation: methyl 3-(azepan-1-yl)-5-(cyclopropanecarbonylamino)pyrazine-2-carboxylate

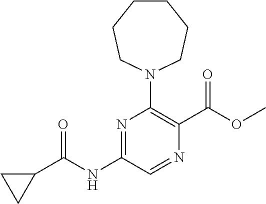 [SMILES: COC(=O)c1ncc(NC(=O)C2CC2)nc1N1CCCCCC1]

To a solution of methyl 3-chloro-5-(cyclopropanecarbonylamino)pyrazine-2-carboxylate (222.0 mg, 0.870 mmol) and N,N-Diisopropylethylamine (0.15 mL, 0.870 mmol) in DMF (2.22 mL) Homopiperidine (0.29 mL, 2.61 mmol) was added and the reaction was stirred at 80° C. for 4h. After this time the reaction was cooled down to RT and diluted with H₂O and the mixture was extracted with AcOEt 3 times. The combined organic fractions were washed with Brine, dried over Na₂SO₄, filtered and concentrated under vacuum. The residue was purified by FC on NH column (eluting from cHex to cHex/AcOEt 1:1) affording the product of formula methyl 3-(azepan-1-yl)-5-(cyclopropanecarbonylamino)pyrazine-2-carboxylate.
Yield: 240 mg

¹ NMR (400 MHz, DMSO-d₆) δ 10.73 (s, 1H), 8.52 (s, 1H), 3.81 (s, 3H), 3.43 (t, 4H), 2.08 (p, 1H), 1.76 (s, 4H), 1.46 (p, 4H), 0.85 (d, 4H); LC-MS: m/z 319.4 (MH+)

Preparation: 3-(azepan-1-yl)-5-(cyclopropanecarbonylamino)pyrazine-2-carboxylic acid

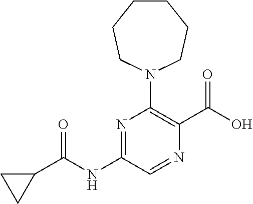 [SMILES: O=C(O)c1ncc(NC(=O)C2CC2)nc1N1CCCCCC1]

To a mixture of methyl 3-(azepan-1-yl)-5-(cyclopropanecarbonylamino)pyrazine-2-carboxylate (240 mg, 0.750 mmol) in THF (1 mL)/Methanol (1 mL) a solution of lithium hydroxide hydrate (63.26 mg, 1.51 mmol) in Water (0.500 mL) was added. The reaction was stirred at 40° C. ON. The day after the reaction was cooled down to RT and acidified with HCI 1N until pH=1. The mixture was concentrated under vacuum. The residue was taken up with H₂O and filtered. The solid was dried under vacuum affording the product of formula 3-(azepan-1-yl)-5-(cyclopropanecarbonylamino)pyrazine-2-carboxylic acid. The product was used in the next step without further purification.
Yield: 230 mg
LC-MS: m/z 305.33 (MH+)
   Preparation: N-[6-(azepan-1-yl)-5-(4-propylpiperazine-1-carbonyl)pyrazin-2-yl]Cyclopropanecarboxamide

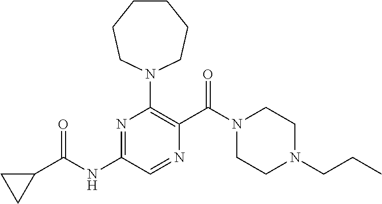 [SMILES: CCCN1CCN(C(=O)c2ncc(NC(=O)C3CC3)nc2N2CCCCCC2)CC1]

### Example 106

To a solution of 3-(azepan-1-yl)-5-(cyclopropanecarbonylamino)pyrazine-2-carboxylic acid (230.0 mg, 0.790 mmol) and [dimethylamino(3-triazolo[4,5-b]pyridinyloxy)methylidene]-dimethylammonium;hexafluorophosphate (299.85 mg, 0.790 mmol) in DMF (3 mL) N,N-Diisopropylethylamine (0.14 mL, 0.790 mmol) was added and the mixture was stirred for 10 min at RT. After this time a solution of 1-propylpiperazine dihydrobromide (228.72 mg, 0.790 mmol) and N,N-Diisopropylethylamine (0.28 mL, 1.58 mmol) in DMF (3 mL) was added and the reaction was stirred ON at RT. The day after the reaction was diluted with a s.s. of NaHCO₃ and extracted with AcOEt 3 times. The organic phases were washed with Brine, dried over Na₂SO₄, filtered and concentrated under vacuum. The residue was purified by SCX first washing with MeOH and then with NH₃ 1M in MeOH and then by FC on C18 (eluting from 5:95 of CH₃CN/aqueous solution adjusted to pH 10 with ammonia to 95: 5 of CH₃CN/aqueous solution adjusted to pH 10 with ammonia) affording the product of formula N-[6-(azepan-1-yl)-5-(4-propylpiperazine-1-carbonyl)pyrazin-2-yl]cyclopropanecarboxamide
Yield: 20 mg

1H NMR (400 MHz, DMSO-d6) δ 10.54 (s, 1 H), 8.44 (s, 1 H), 3.60 (br s, 2 H), 3.53-3.43 (m, 4 H), 3.37 (br t, 2 H), 2.37 (br s, 2 H), 2.34-2.28 (m, 2 H), 2.28-2.21 (m, 2 H), 2.10-1.97 (m, 1 H), 1.73 (br s, 4 H), 1.54-1.36 (m, 6 H), 0.90-0.79 (m, 7 H); LC-MS: m/z 415.4 (MH+).

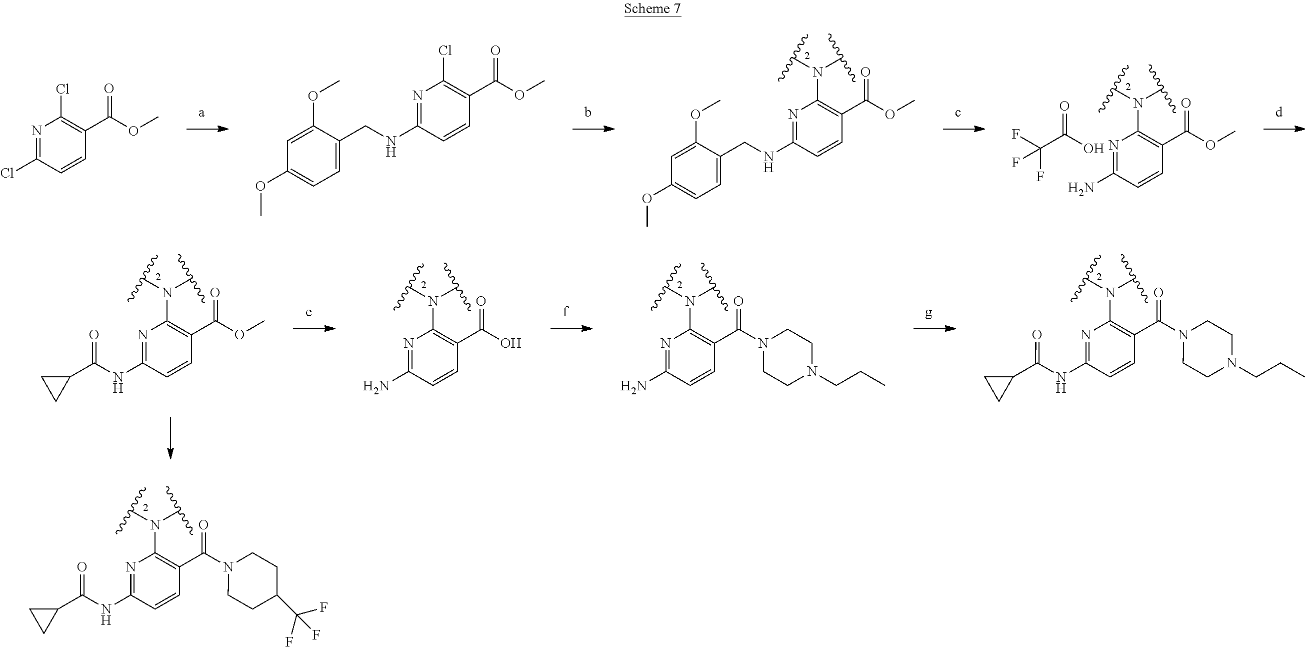

Preparation: methyl 2-chloro-6-[(2,4-dimethoxyphenyl)methylamino]pyridine-3-carboxylate

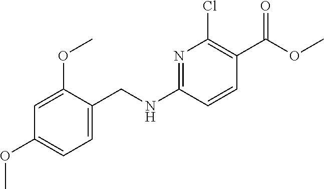 [SMILES: COC(=O)c1ccc(NCc2ccc(OC)cc2OC)nc1Cl]

A mixture of 2,6-dichloro-3-pyridinecarboxylic acid methyl ester (1.2 g, 5.82 mmol), (2,4-dimethoxyphenyl)methanamine (0.88 mL, 5.82 mmol) and cesium fluoride (0.88 g, 5.82 mmol) in DMF (19.41 mL) was stirred at 80° C. for 3 h. After this time the reaction was cooled down to RT diluted with H₂O and extracted with AcOEt 3 times. The organic phase was washed with Brine, dried, filtered, and concentrated under vacuum. The residue was purified by FC on NH column (eluting from cHex to cHex/AcOEt 1:1) affording the product of formula methyl 2-chloro-6-[(2,4-dimethoxyphenyμmethylamino]pyridine-3-carboxylate.
Yield: 726 mg

1H NMR (500 MHz, DMSO-d6) δ 7.93 (br s, 1 H), 7.87 (br d, 1 H), 7.13 (br d, 1 H), 6.57 (d, 1 H), 6.54-6.45 (m, 1 H), 6.48 (dd, 1 H), 4.36 (br s, 2 H), 3.80 (s, 3 H), 3.75 (s, 3 H), 3.74 (s, 3 H); LC-MS: m/z 337.2 (MH+).

Preparation: methyl 2-(azepan-1-yl)-6-[(2,4-dimethoxypheny)methylamino]pyridine-3-carboxylate

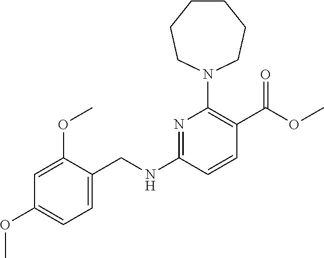 [SMILES: COC(=O)c1ccc(NCc2ccc(OC)cc2OC)nc1N1CCCCCC1]

A mixture of methyl 2-chloro-6-[(2,4-dimethoxyphenyμmethylamino]pyridine-3-carboxylate (726.0 mg, 2.16 mmol), N,N-Diisopropylethylamine (1.12 mL, 4.31 mmol) and Homopiperidine (0.5 mL, 3.80 mmol) in DMF (14 mL) was stirred for 5 h at 120° C. After this time the reaction was cooled down to RT and H₂O was added. The mixture was then extracted with AcOEt 3 times. The combined organic fractions were washed with Brine, filtered and concentrated under vacuum. The crude was purified by FC on NH column (eluting from 100% of cHex to cHex/AcOEt 1:1) affording the product of methyl 2-(azepan-1-yl)-6-[(2,4-dimethoxyphenyl)methylamino]pyridine-3-carboxylate
Yield: 543 mg

1H NMR (400 MHz, DMSO-d6) δ 7.54 (d, 1H), 7.14-7.06 (m, 2H), 6.53 (d, 1H), 6.44 (dd, 1H), 5.84 (d, 1H), 4.38 (d, 2H), 3.78 (s, 3H), 3.72 (s, 3H), 3.64 (s, 3H), 3.31-3.28 (m, 4H), 1.73-1.63 (m, 4H), 1.41-1.36 (m, 4H); LC-MS: m/z 400.22 (MH+).

Preparation: methyl 6-[(2,4-dimethoxyphenyl)methylamino]-2-pyrrolidin-1-ylpyridine-3-carboxylate

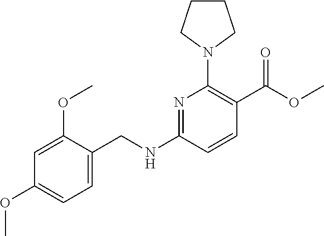 [SMILES: COC(=O)c1ccc(NCc2ccc(OC)cc2OC)nc1N1CCCC1]

A mixture of methyl 2-chloro-6-[(2,4-dimethoxyphenyl)methylamino]pyridine-3-carboxylate (820.0 mg, 2.43 mmol), N,N-Diisopropylethylamine (0.85 mL, 4.87 mmol) and pyrrolidine (0.41 mL, 4.87 mmol) in DMF (16.4 mL) was heated to 80° C. for 5 h. After this time the reaction was cooled down to RT and H₂O was added. The mixture was then extracted with AcOEt 3 times. The combined organic fractions were washed with Brine, dried over Na₂SO₄, filtered and concentrated under vacuum. The residue was purified by FC on NH column (eluting from 100% of cHex to cHex/AcOEt 3:7) affording the compound of formula methyl 6-[(2,4-dimethoxyphenyl)methylamino]-2-pyrrolidin-1-ylpyridine-3-carboxylate
Yield: 900 mg.
LC-MS: 372.2 (MH+).
   Preparation: [6-(azepan-1-yl)-5-methoxycarbonylpyridin-2-yl]azanium; 2,2,2-trifluoroacetate

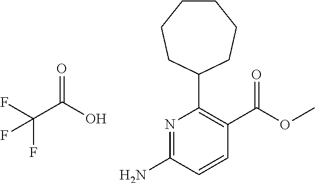 [SMILES: O=C(O)C(F)(F)F ; COC(=O)c1ccc(N)nc1C1CCCCCC1]

To a mixture of methyl 2-(azepan-1-yl )-6-[(2,4-dimethoxyphenyl)methylamino]pyridine-3-carboxylate (200.0 mg, 0.500 mmol) in DCM (2 mL) Trifluoroacetic acid (0.19 mL, 2.5 mmol) was added. The reaction was stirred at RT for 2 h. After this time the reaction was concentrated under vacuum affording the product of formula [6-(azepan-1-yl)-5-methoxycarbonylpyridin-2-yl]azanium; 2,2,2-trifluoroacetate
Yield: 200 mg
LC-MS: m/z 250.10 (MH+).
   Preparation: (5-methoxycarbonyl-6-pyrrolidin-1-ylpyridin-2-yl)azanium; 2,2,2-trifluoroacetate

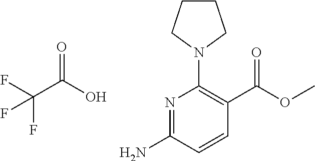 [SMILES: O=C(O)C(F)(F)F ; COC(=O)c1ccc(N)nc1N1CCCC1]

To a solution of methyl 6-[(2,4-dimethoxyphenyl)methylamino]-2-pyrrolidin-1-ylpyridine-3-carboxylate (900.0 mg, 2.42 mmol) in DCM (5 mL) Trifluoroacetic acid (0.93 mL, 12.12 mmol) was added. The reaction was stirred at RT ON. The day after the reaction was concentrated under vacuum affording the product of formula (5-methoxycarbonyl-6-pyrrolidin-1-ylpyridin-2-yl)azanium; 2,2,2-trifluoroacetate
Yield: 800 mg
LC-MS: m/z 222.0 (MH+)
   Preparation: methyl 2-(azepan-1-yl)-6-(cyclopropanecarbonylamino)pyridine-3-carboxylate

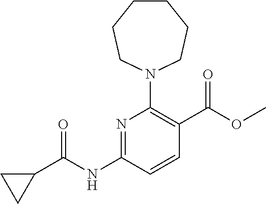 [SMILES: COC(=O)c1ccc(NC(=O)C2CC2)nc1N1CCCCCC1]

A mixture of [6-(azepan-1-yl )-5-methoxycarbonylpyridin-2-yl]azanium; 2,2,2-trifluoroacetate (200.0 mg, 0.550 mmol) and cyclopropanecarbonyl chloride (0.05 mL, 0.550 mmol) in Pyridine (2.752 mL) was heated to 80° C. for 3 h. After this time the reaction was cooled down to RT, H₂O was added and the mixture was filtered. The solid was washed with H₂O and dried under vacuum affording the product of formula methyl 2-(azepan-1-yl )-6-(cyclopropanecarbonylamino)pyridine-3-carboxylate, which was used in the next step without further purification.
Yield: 165 mg

¹ NMR (400 MHz, DMSO-d₆) δ 10.37 (s, 1H), 7.78 (d, 1H), 7.32 (d, 1H), 3.75 (s, 3H), 3.40 (t, 4H), 2.13-2.00 (m, 1H), 1.76 (s, 4H), 1.45 (q, 4H), 0.80 (d, 4H).

Preparation: methyl 6-(cyclopropanecarbonylamino)-2-pyrrolidin-1-ylpyridine-3-carboxylate

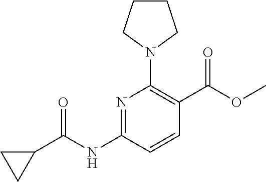 [SMILES: COC(=O)c1ccc(NC(=O)C2CC2)nc1N1CCCC1]

A mixture of (5-methoxycarbonyl-6-pyrrolidin-1-ylpyridin-2-yl)azanium; 2,2,2-trifluoroacetate (800.0 mg, 2.39 mmol) and cyclopropanecarbonyl chloride (0.22 mL, 2.39 mmol) in Pyridine (11.01 mL) was heated to 80° C. for 3 h. After this time the reaction was cooled down to RT, H₂O was added and the so obtained suspension was filtered. The solid was washed with H₂O and dried under vacuum affording the product of formula methyl 6-(cyclopropanecarbonylamino)-2-pyrrolidin-1-ylpyridine-3-carboxylate, which was used in the next step without further purification.
Yield: 275 mg

¹ NMR (400 MHz, DMSO-d₆) δ 10.44 (s, 1H), 7.82 (d, 1H), 7.35 (d, 1H), 3.50-3.28 (m, 7H), 2.14-2.04 (m, 1H), 1.93-1.77 (m, 4H), 0.86-0.71 (m, 4H); LC-MS: m/z 290.0 (MH+).

Preparation: N-[6-pyrrolidin-1-yl-5-[4-(trifluoromethyl)piperidine-1-carbonyl]pyridin-2-yl]Cyclopropanecarboxamide

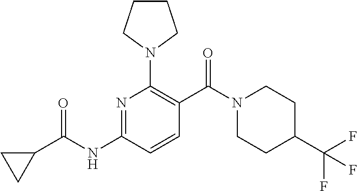 [SMILES: O=C(Nc1ccc(C(=O)N2CCC(C(F)(F)F)CC2)c(N2CCCC2)n1)C1CC1]

### Example 107

A solution of 4-(trifluoromethyl)piperidine (0.19 mL, 0.380 mmol) in Toluene (2 mL) was cooled to 0 C and after 10 min a solution of trimethylalumane (0.19 mL, 0.380 mmol) 2M in toluene was added under N2. The mixture was stirred at the same temperature for 30 minutes and added to a mixture of methyl 6-(cyclopropanecarbonylamino)-2-pyrrolidin-1-ylpyridine-3-carboxylate (100.0 mg, 0.350 mmol) in Toluene (2 mL). The reaction was heated to reflux for 72 h. After this time the reaction was cooled down to RT and concentrated under vacuum. The residue was purified by FC on NH column (from 100% of DCM to DCM/MeOH 95:5) and further purified on RP using basic conditions (eluting from 100% of Water +0.1% of NH₄OH to 100% of CH₃CN) affording the product of formula N-[6-pyrrolidin-1-yl-5-[4-(trifluoromethyl)piperidine-1-carbonyl]pyridin-2-yl]cyclopropanecarboxamide
Yield: 9.5 mg

1H NMR (500 MHz, DMSO-d6) δ 10.37-10.18 (m, 1 H), 7.40-7.27 (m, 2 H), 4.69-3.54 (m, 2 H), 3.51- 3.16 (m, 4 H), 3.18-2.68 (m, 2 H), 2.68-2.51 (m, 1 H), 2.13-2.01 (m, 1 H), 1.94-1.60 (m, 6 H), 1.46-1.22 (m, 2 H), 0.88-0.70 (m, 4 H); LC-MS: m/z 411.2 (MH+).

Preparation: 6-amino-2-(azepan-1-yl)pyridine-3-carboxylic acid; formic acid

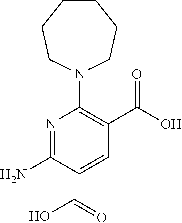

To solution of methyl 2-(azepan-1-y0-6-(cyclopropanecarbonylamino)pyridine-3-carboxylate (165.0 mg, 0.520 mmol) in THF (1.752 mL)/Methanol (0.701 mL) a solution of lithium hydroxide hydrate (43.63 mg, 1.04 mmol) in Water (0.701 mL) was added. The reaction was stirred ON at 50° C. The reaction was cooled down to RT, acidified with HCI 1 N until pH =1 and concentrated under vacuum. The residue was purified by FC on C18 column (eluting from H₂O/CH₃CN 95:5 +0.1% of formic acid to H₂O/CH₃CN 5:95 +0.1% of FA) affording the product of formula 6-amino-2-(azepan-1-yl)pyridine-3-carboxylic acid formic acid
Yield: 180 mg
LC-MS: m/z 236.10 (MH+).
   Preparation: [6-amino-2-(azepan-1-yppyridin-3-yl]-(4-propylpiperazin-1-yl)methanone

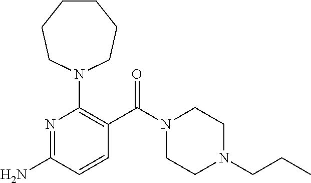 [SMILES: CCCN1CCN(C(=O)c2ccc(N)nc2N2CCCCCC2)CC1]

To a mixture of [dimethylamino(3-triazolo[4,5-b]pyridinyloxy)methylidene]-dimethylammonium hexafluorophosphate (270.33 mg, 0.710 mmol), 6-amino-2-(azepan-1-yl)pyridine-3-carboxylic acid formic acid (200.0 mg, 0.710 mmol) and 1-propylpiperazine dihydrobromide (412.41 mg, 1.42 mmol) in DMF (6 mL) N,N-Diisopropylethylamine (0.74 mL, 4.27 mmol) was added and the reaction was stirred at RT ON. The day after the mixture reaction was purified by FC on RP using acidic conditions (eluting from CH₃CN/H₂O 5:95 +0.1% of formic acid to CH₃CN/H₂O 95:5 +0.1% of formic acid) and it was further purified by FC on RP using basic conditions (eluting from 5:95 of CH₃CN/aqueous solution adjusted to pH 10 with ammonia to 95: 5 of CH₃CN/aqueous solution adjusted to pH 10 with ammonia) affording the product of formula [6-amino-2-(azepan-1-yppyridin-3-yl]-(4-propylpiperazin-1-yl)methanone.
Yield: 33 mg
LC-MS: m/z 346.28 (MH+).
   Preparation: N-[6-(azepan-1-yl)-5-(4-propylpiperazine-1-carbonyppyridin-2-yl]cyclopropanecarboxamide

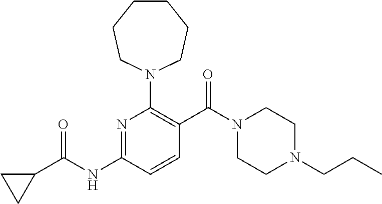 [SMILES: CCCN1CCN(C(=O)c2ccc(NC(=O)C3CC3)nc2N2CCCCCC2)CC1]

### Example 108

To a solution of [6-amino-2-(azepan-1-yppyridin-3-yl]-(4-propylpiperazin-1-yl)methanone (33.0 mg, 0.100 mmol) in Pyridine (2 mL), cyclopropanecarbonyl chloride (0.01 mL, 0.100 mmol) was added and the reaction was stirred at 80° C. for 2 h. After this time the reaction was cooled down to RT and concentrated under vacuum. The residue was purified by SCX first washing with MeOH and then with NH3 1M in MeOH and then by FC on RP using acid conditions (eluting from 5:95 of CH₃CN/H₂O +0.1% of formic acid to 95: 5 of CH₃CN/H₂O +1% of FA) and it was further purified by SCX first washing with MeOH and then with NH3 1M in MeOH affording the product of formula N-[6-(azepan-1-yl)-5-(4-propylpiperazine-1-carbonyppyridin-2-yl]cyclopropanecarboxamide
Yield: 14 mg

1H NMR (400 MHz, DMSO-d6) δ 10.22 (s, 1 H), 7.37-7.23 (m, 2 H), 3.58 (br t, 2 H), 3.54-3.35 (m, 4 H), 3.26 (br t, 2 H), 2.42-2.18 (m, 6 H), 2.05 (quin, 1 H), 1.82-1.62 (m, 4 H), 1.58-1.35 (m, 6 H), 0.85 (t, 3 H), 0.81-0.75 (m, 4 H); LC-MS: m/z 414.28 (MH+).

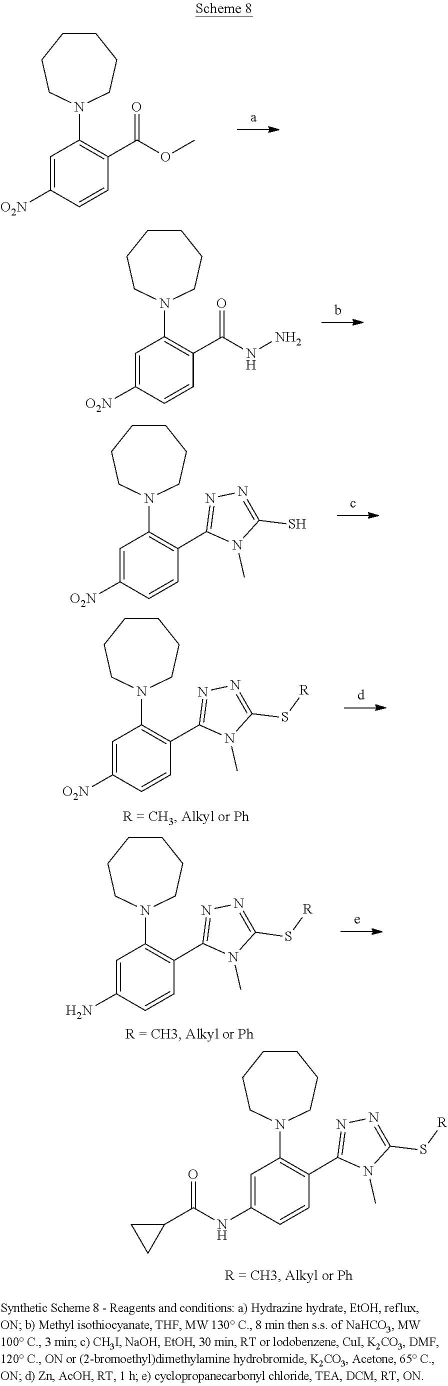

Preparation: 2-(azepan-1-yl)-4-nitrobenzohydrazide

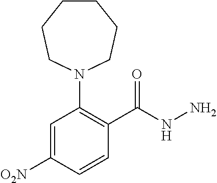 [SMILES: NNC(=O)c1ccc([N+](=O)[O-])cc1N1CCCCCC1]

To a solution of methyl 2-(azepan-1-yl)-4-nitrobenzoate (1.89 g, 6.79 mmol) in Ethanol (15.79 mL) hydrazine hydrate (2.56 mL, 33.96 mmol) was added and the reaction was heated to reflux and stirred ON. The day after the reaction mixture was cooled down to RT and concentrated under reduced pressure. The resulting material was purified by FC on NH column (eluting from DCM/AcOEt 95:5 to DCM/ACOEt 6:4) affording the product of formula 2-(azepan-1-yl)-4-nitrobenzohydrazide.
Yield: 690 mg

¹ NMR (400 MHz, DMSO-d₆) δ 9.62 (s, 1H), 7.57 (d, 1H), 7.48 (dd, 1H), 7.31 (d, 1H), 4.47 (d, 2H), 3.37 (dd, 4H), 1.73 (p, 4H), 1.51 (p, 4H); LC-MS: m/z 279.13 (MH+).

Preparation: 5-[2-(azepan-1-yl)-4-nitrophenyl]-4-methyl-1,2,4-triazole-3-thiol

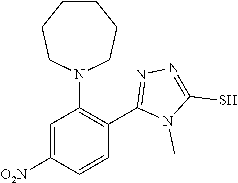 [SMILES: Cn1c(S)nnc1-c1ccc([N+](=O)[O-])cc1N1CCCCCC1]

To a solution of 2-(azepan-1-yl)-4-nitrobenzohydrazide (550.0 mg, 1.98 mmol) in THF (10 mL) Methyl isothiocyanate (0.15 mL, 2.17 mmol) was added and the mixture was stirred at 130° C. under MW conditions for 8 min. The organic solution was concentrated and 12.5 ml of s.s. of NaHCO₃ was added and the mixture reaction was stirred at 100° C. under MW conditions for 3 minutes. Then the aqueous solution was neutralized with HCI 2M and extracted with AcOEt 3 times. The organic fractions were washed with Brine, dried over Na₂SO₄, filtered and concentrated under vacuum. The residue was purified by FC on silica gel (eluting from 100% of cHex to cHex/AcOEt 7:3) affording the product of formula 5-[2-(azepan-1-yl)-4-nitrophenyl]-4-methyl-1,2,4-triazole-3-thiol
Yield: 420 mg

¹ NMR (400 MHz, Chloroform-d) δ 11.14 (s, 1H), 7.95 (d, 1H), 7.76 (dd, 1H), 7.45 (d, 1H), 3.39 (s, 3H), 3.28-3.22 (m, 4H), 1.68 (q, 4H), 1.63-1.59 (s, 4H); LC-MS: m/z 334.12 (MH+)

Preparation: 1-[2-(4-methyl-5-methylsulfanyl-1,2,4-tria3407zol-3-y0-5-nitrophenyl]azepane

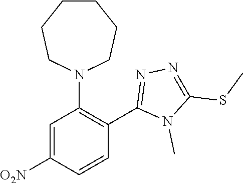 [SMILES: CSc1nnc(-c2ccc([N+](=O)[O-])cc2N2CCCCCC2)n1C]

5[2-(azepan-1-yl)-4-nitrophenyl]-4-methyl-1,2,4-triazole-3-thiol (250.0 mg, 0.750 mmol), sodium hydroxide (1.57 mL, 1.57 mmol) 1M solution and iodomethane (0.07 mL, 1.2 mmol) were mixed in Ethanol (0.375 mL) and the reaction was stirred for 30 min at room temperature. The reaction mixture was diluted with NaHCO₃ and extracted with EtOAc 3 times. The organic layers were collected together, washed with Brine, dried over Na₂SO₄, filtered and concentrated under vacuum affording the product of formula 1-[2-(4-methyl-5-methylsulfanyl-1,2,4-triazol-3-y0-5-nitrophenyl]azepane. The product was used in the next step without further purification.
Yield: 224 mg

¹ NMR (400 MHz, Chloroform-d) δ 7.92 (d, 1H), 7.77 (dd, 1H), 7.54 (d, 1H), 3.34 (s, 3H), 3.21-3.14 (m, 4H), 2.81 (s, 3H), 1.65-1.41 (m, 8H); LC-MS: m/z 348.15 (MH+)

Preparation: 1-[2-(4-methyl-5-phenylsulfanyl-1,2,4-triazol-3-yl)-5-nitrophenyl]azepane

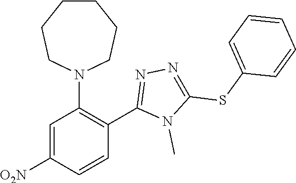 [SMILES: Cn1c(Sc2ccccc2)nnc1-c1ccc([N+](=O)[O-])cc1N1CCCCCC1]

To a solution of lodobenzene (0.03 mL, 0.240 mmol), copper (I) iodide (1.9 mg, 0.010 mmol) and potassium carbonate (35.57 mg, 0.260 mmol) in dry DMF (2 mL) a solution of 5-[2-(azepan-1-yl)-4-nitrophenyl]-4-methyl-1,2,4-triazole-3-thiol (66 mg, 0.198 mmol) in DMF (0.2 mL) was added. The resulting mixture was degassed with Schlenk line technique and stirred at 120° C. ON. The day after the reaction mixture was cooled down to RT diluted with a ss of NaHCO₃ and extracted with AcOEt, washed with Brine, dried over Na₂SO₄, filtered and concentrated under reduced pressure affording the product of formula 142-(4-methyl-5-phenylsulfanyl-1,2,4-triazol-3-y0-5-nitrophenyl]azepane, which was used in the next step without further purification.
Yield: 40 mg
LC-MS: m/z 410.14 (MH+)
   Preparation: 2-[[5-[2-(azepan-1-yl)-4-nitrophenyl]-4-methyl-1,2,4-triazol-3-yl]sulfanyl]-N,N-dimethylethanamine

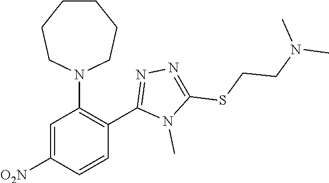 [SMILES: CN(C)CCSc1nnc(-c2ccc([N+](=O)[O-])cc2N2CCCCCC2)n1C]

To a mixture of 5-[2-(azepan-1-yl )-4-nitrophenyl]-4-methyl-1,2,4-triazole-3-thiol (100.0 mg, 0.300 mmol), (2-bromoethyl)dimethylamine hydrobromide in Acetone (3 mL), potassium carbonate (82.91 mg, 0.600 mmol) was added. The reaction was stirred at 65° C. ON. After this time the reaction mixture was cooled down to RT, diluted with s. s. of NaHCO₃ and extracted with DCM 3 times. The organic phase was washed with Brine, dried over Na₂SO₄, filtered and concentrated under vacuum affording the formula product 2-[[5-[2-(azepan-1-yl)-4-nitrophenyl]-4-methyl-1,2,4-triazol-3-yl]sulfanyl]-N,N-dimethylethanamine which was used in the next step without further purification.
Yield: 120 mg
LC-MS: m/z 405.15 (MH+).
   Preparation: 3-(azepan-1-yl)-4-(4-methyl-5-methylsulfanyl-1,2,4-triazol-3-yl)aniline

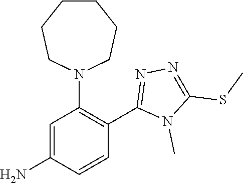 [SMILES: CSc1nnc(-c2ccc(N)cc2N2CCCCCC2)n1C]

To a solution of 1-[2-(4-methyl-5-methylsulfanyl-1,2,4-triazol-3-yl)-5-nitrophenyl]azepane (100.0 mg, 0.290 mmol) in dry Acetic acid (3 mL) zinc (94.09 mg, 1.44 mmol) was slowly added. The resulting suspension was stirred at room temperature for 1h. The reaction mixture was filtered and washed with AcOEt and the organic phase concentrated under reduced pressure. The resulting material was taken up with a s. s. of NaHCO₃ and extracted with AcOEt 3 times. The combined organic fractions were washed with Brine, dried over Na₂SO₄, filtered and concentrated under reduced pressure affording the product of formula 3-(azepan-1-yl)-4-(4-methyl-5-methylsulfanyl-1,2,4-triazol-3-yl)aniline, which was used in the next step without further purification.
Yield: 60 mg
LC-MS: m/z 318.20 (MH+)
   Preparation: 3-(azepan-1-yl)-4-(4-methyl-5-phenylsulfanyl-1,2,4-triazol-3-yl)aniline

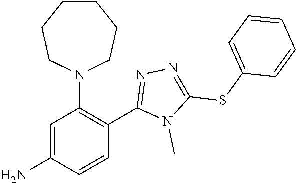 [SMILES: Cn1c(Sc2ccccc2)nnc1-c1ccc(N)cc1N1CCCCCC1]

The synthesis of the title compound was effected analogously to the synthesis of 3-(azepan-1-yl)-4-(4-methyl-5-methylsulfanyl-1,2,4-triazol-3-yl)aniline wherein 1-[2-(4-methyl-5-phenylsulfanyl-1,2,4-triazol-3-y0-5-nitrophenyl]azepane was used instead of 1-[2-(4-methyl-5-methylsulfanyl-1,2,4-triazol-3-yl)-5-nitrophenyl]azepane. The title compound was obtained in a yield of 71% (80 mg).
LC-MS: m/z 380.18 (MH+)
   Preparation: 3-(azepan-1-yl)-4-[5-[2-(dimethylamino)ethylsulfanyl]-4-methyl-1,2,4-triazol-3-yl]aniline

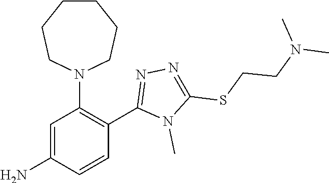 [SMILES: CN(C)CCSc1nnc(-c2ccc(N)cc2N2CCCCCC2)n1C]

The synthesis of the title compound was effected analogously to the synthesis of 3-(azepan-1-yl)-4-(4-methyl-5-methylsulfanyl-1,2,4-triazol-3-yl)aniline wherein 2[[5-[2-(azepan-1-yl)-4-nitrophenyl]-4-methyl-1,2,4-triazol-3-yl]sulfanyl]-N,N-dimethylethanamine was used instead of 1-[2-(4-methyl-5-methylsulfanyl-1,2,4-triazol-3-yl)-5-nitrophenyl]azepane. The title compound was obtained in a yield of 67% (50 mg).
LC-MS: m/z 375.27 (MH+).
   Preparation: N-[3-(azepan-1-yl)-4-(4-methyl-5-methylsulfanyl-1,2,4-triazol-3-yl)phenyl]Cyclopropanecarboxamide

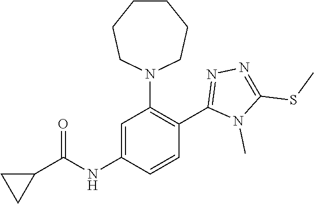 [SMILES: CSc1nnc(-c2ccc(NC(=O)C3CC3)cc2N2CCCCCC2)n1C]

### Example 109

A solution of 3-(azepan-1-yl )-4-(4-methyl-5-methylsulfanyl-1,2,4-triazol-3-yl)aniline (60.0 mg, 0.190 mmol) and triethylamine (0.08 mL, 0.570 mmol) in DCM (0.945 mL) was cooled to 0° C. and cyclopropanecarbonyl chloride (0.04 mL, 0.470 mmol) was added. The mixture was allowed to reach room temperature and stirred ON. The day after the reaction mixture was diluted with DCM, washed with s. s. of NaHCO₃, dried over a phase separator and concentrated under reduced pressure. The resulting material was purified by FC on RP using acidic conditions (eluting from CH₃CN/H₂O 5:95 +0.1% of formic acid to CH₃CN/H₂O 95:5 +0.1% of formic acid) affording the product of formula N-[3-(azepan-1-yl)-4-(4-methyl-5-methylsulfanyl-1,2,4-triazol-3-yl)phenyl]Cyclopropanecarboxamide.
Y₁led: 16.8 mg

¹ NMR (400 MHz, Chloroform-d) δ 7.76 (s, 1H), 7.62 (s, 1H), 7.28 (s, 1H), 6.79 (dd, 1H), 3.34 (s, 3H), 3.12 (t, 4H), 2.77 (s, 3H), 1.54 (s, 9H), 1.18-1.09 (m, 2H), 0.90 (dt, 2H); LC-MS: m/z 386.20 (MH+).

Preparation: N-[3-(azepan-1-yl)-4-(4-methyl-5-phenylsulfanyl-1,2,4-triazol-3-yl)phenyl]cyclopropanecarboxamide

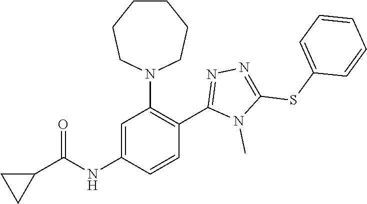 [SMILES: Cn1c(Sc2ccccc2)nnc1-c1ccc(NC(=O)C2CC2)cc1N1CCCCCC1]

### Example 110

The synthesis of the title compound was effected analogously to the synthesis of the compound of Example 109 (N-[3-(azepan-1-yl)-4-(4-methyl-5-methylsulfanyl-1,2,4-triazol-3-yl)phenyl]Cyclopropanecarboxamide) wherein 3-(azepan-1-yl)-4-(4-methyl-5-phenylsulfanyl-1,2,4-triazol yl)aniline was used instead of 3-(azepan-1-yl)-4-(4-methyl-5-methylsulfanyl-1,2,4-triazol-3-yl)aniline. The title compound was obtained in a yield of 12% (19 mg).

¹ NMR (400 MHz, Chloroform-d) δ 7.73 (d, 2H), 7.48-7.43 (m, 2H), 7.39-7.30 (m, 4H), 6.79 (dd, 1H), 3.33 (s, 3H), 3.03 (d, 4H), 1.57 (s, 1H), 1.45-1.32 (m, 8H), 1.16-1.05 (m, 2H), 0.89 (dt, 2H); LC-MS: m/z 448.17.

Preparation: N-[3-(azepan-1-yl)-4-[5-[2-(dimethylamino)ethylsulfanyl]-4-methyl-1,2,4-triazol-3-yl]phenyl]Cyclopropanecarboxamide

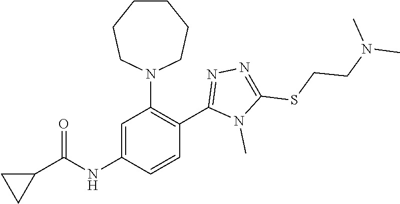 [SMILES: CN(C)CCSc1nnc(-c2ccc(NC(=O)C3CC3)cc2N2CCCCCC2)n1C]

### Example 111

The synthesis of the title compound was effected analogously to the synthesis of the compound of Example 109 (N-[3-(azepan-1-yl)-4-(4-methyl-5-methylsulfanyl-1,2,4-triazol-3-yl)phenyl]Cyclopropanecarboxamide) wherein 3-(azepan-1-yl)-4-[5-[2-(dimethylamino)ethylsulfanyl]-4-methyl-1,2,4-triazol-3-yl]aniline was used instead of 3-(azepan-1-yl)-4-(4-methyl-5-methylsulfanyl-1,2,4-triazol-3-yl)aniline. The title compound was obtained in a yield of 9% (6.3 mg).

1H NMR (400 MHz, CHLOROFORM-d) δ 7.73 (br s, 1 H), 7.58-7.47 (m, 1 H), 7.28 (s, 1 H), 6.78 (dd, 1 H), 3.45 (t, 2 H), 3.32 (s, 3 H), 3.19-3.04 (m, 4 H), 2.74 (s, 2 H), 2.31 (s, 6 H), 1.52 (br s, 9 H), 1.19-1.05 (m, 2 H), 0.96-0.83 (m, 3 H); LC-MS: 443.18 (MH+).

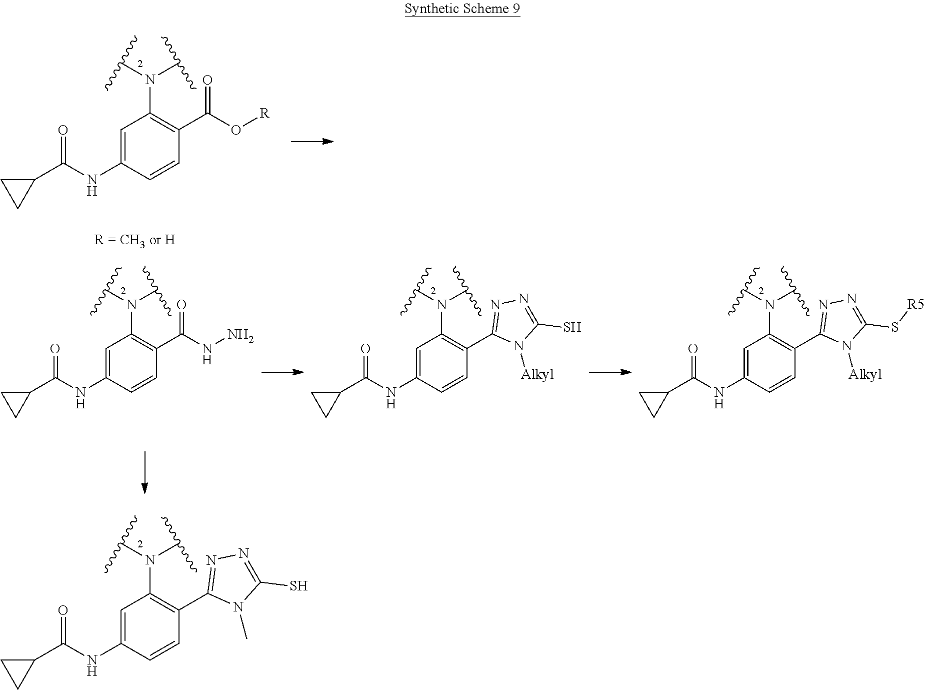

Preparation: N-[3-(3,5-dimethylpiperidin-1-yl)-4-(hydrazinecarbonyl)phenyl]cyclopropanecarboxamide

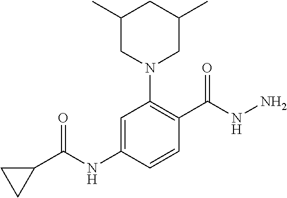 [SMILES: CC1CC(C)CN(c2cc(NC(=O)C3CC3)ccc2C(=O)NN)C1]

To a solution of methyl 4-(cyclopropanecarbonylamino)-2-(3,5-dimethylpiperidin-1-yl)benzoate (1.5 g, 4.54 mmol) in Ethanol (10.56 mL) hydrazine hydrate (3.43 mL, 45.4 mmol) was added. The resulting solution was stirred at 85° C. for 4h. After this time the reaction was cooled down to RT and concentrated under reduced pressure affording the formula product N-[3-(3,5-dimethylpiperidin-1-yl)-4-(hydrazinecarbonyl)phenyl]cyclopropanecarboxamide as mixture of syn/anti isomers.
Yield: 1.5 g
LC-MS: 331.22 (MH+).

### Preparation: N-[3-(azepan-1-yl)-4-(hydrazinecarbonyl)phenyl]cyclopropanecarboxamide

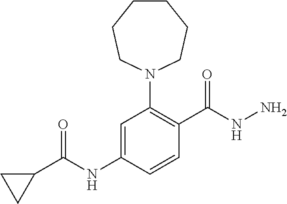 [SMILES: NNC(=O)c1ccc(NC(=O)C2CC2)cc1N1CCCCCC1]

To a solution of 2-(azepan-1-ium-1-yl)-4-(cyclopropanecarbonylamino)benzoic acid chloride (250.0 mg, 0.740 mmol) in DMF (7.018 mL) HATU=[dimethylamino(3-triazolo[4,5-b]pyridinyloxy)methylidene]-dimethylammonium hexafluorophosphate (308.6 mg, 0.810 mmol) and hydrazine hydrochloride (252.74 mg, 3.69 mmol) were added. This solution was stirred for 30 min at room temperature and a solution of N,N-Diisopropylethylamine (333.75 mg, 2.58 mmol) in DMF (2 mL) was slowly added and stirred at room temperature ON. The day after the reaction mixture was diluted with DCM and washed three times with a s.s. of NaHCO₃, washed with Brine, dried through a phase separator and concentrated under vacuum. The residue was purified by FC on RP using acidic conditions (eluting from CH₃CN/H₂O 5:95 +0.1% of formic acid to CH₃CN/H₂O 95:5 +0.1% of formic acid) affording the product of formula N-[3-(azepan-1-yl)-4-(hydrazinecarbonyl)phenyl]cyclopropanecarboxamide.
Yield: 60 mg

¹ NMR (400 MHz, DMSO-d₆) δ 10.22 (s, 1H), 9.98 (s, 1H), 7.47 (d, 1H), 7.38 (d, 1H), 7.11 (dd, 1H), 4.41 (s, 2H), 3.20-3.10 (m, 4H), 1.79-1.71 (m, 5H), 1.59 (dt, 4H), 0.89-0.67 (m, 4H); LC-MS: m/z 317.23 (MH+)

Preparation: N-[4-(hydrazinecarbonyl)-3-pyrrolidin-1-ylphenyl]cyclopropanecarboxamide

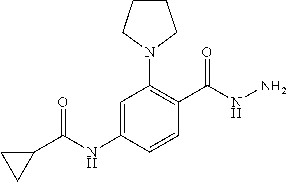 [SMILES: NNC(=O)c1ccc(NC(=O)C2CC2)cc1N1CCCC1]

The synthesis of the title compound was effected analogously to the synthesis of N-[3-(azepan-1-yl)-4-(hydrazinecarbonyl)phenyl]cyclopropanecarboxamide wherein 4-(cyclopropanecarbonylamino)-2-pyrrolidin-1-ylbenzoic acid was used instead of 2-(azepan-1-ium-1-yl)-4-(cyclopropanecarbonylamino)benzoic acid chloride. The title compound was obtained in a yield of 99% (1 g)

1H NMR (400 MHz, DMSO-d6) δ 10.09 (s, 1H), 9.23 (s, 1H), 7.07-7.00 (m, 2H), 6.89 (dd, 1H), 3.20-3.09 (m, 4H), 1.91— 1.81 (m, 4H), 1.77 (tt, 2H), 0.83-0.72 (m, 4H); LC-MS: m/z 289.16 (MH+).

Preparation: N-[3-(azepan-1-yl)-4-(4-methyl-5-phenyl-1,2,4-triazol-3-yl)phenyl]cyclopropanecarboxamide

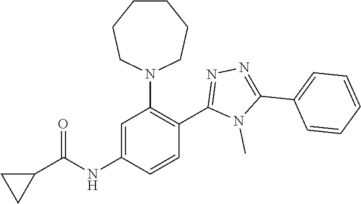 [SMILES: Cn1c(-c2ccccc2)nnc1-c1ccc(NC(=O)C2CC2)cc1N1CCCCCC1]

### Example 112

A solution of N-methylbenzamide (25.63 mg, 0.190 mmol) and 2,6-dimethylpyridine (0.04 mL, 0.380 mmol) in dry DCM (0.948 mL) was cooled to 0° C. and after 10 minutes oxalyl dichloride (0.02 mL, 0.190 mmol) was added carefully under N2 atmosphere and the resulting mixture was stirred at the same temperature for 30 minutes. After this time N-[3-(azepan-1-yl)-4-(hydrazinecarbonyl)phenyl]cyclopropanecarboxamide (60.0 mg, 0.190 mmol) was added and the reaction mixture was allowed to reach R.T. and stirred for 2h. After this time the reaction mixture was concentrated, the residue was taken up with 5 mL of s. s. of NaHCO₃ and the mixture was refluxed ON. The day after the reaction mixture acidified until pH =2 with HCI 1N and concentrated under vacuum. The residue was purified by FC on RP using basic conditions (eluting from 100% of Water +0.1% of NH₄OH to 100% of CH₃CN) affording the formula compound.
Yield: 5.8 mg

1H NMR (400 MHz, DMSO-d6) δ ppm 10.36-10.27 (m, 1 H), 7.77-7.71 (m, 2 H), 7.62-7.54 (m, 4 H), 7.22-7.18 (m, 2 H), 3.44-3.38 (m, 3 H), 3.07-2.99 (m, 4 H), 1.85-1.74 (m, 1 H), 1.53-1.39 (m, 8 H), 0.86 -0.76 (m, 4 H); LC-MS: m/z 416.27 (MH+).

Preparation: N-[3-(azepan-1-yl)-4-(4,5-dimethyl-1,2,4-triazol-3-yl)phenyl]cyclopropanecarboxamide

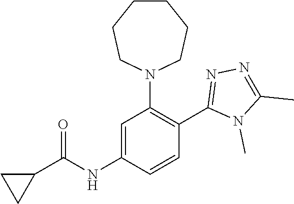 [SMILES: Cc1nnc(-c2ccc(NC(=O)C3CC3)cc2N2CCCCCC2)n1C]

### Example 113

A solution of N-methylacetamide (36.96 mg, 0.510 mmol) and 2,6-dimethylpyridine (0.12 mL, 1.01 mmol) in DCM (2.528 mL) was cooled to 0° C. and after 10 min oxalyl dichloride (0.04 mL, 0.510 mmol) was carefully added under nitrogen atmosphere. The resulting mixture was stirred at the same temperature for 30 min and then N-[3-(azepan-1-yl) (hydrazinecarbonyl)phenyl]cyclopropanecarboxamide (160.0 mg, 0.510 mmol) was added. The reaction was warmed to RT and stirred for 3.5 h. Volatiles were removed under reduced pressure and the resulting material was taken up with 3.5 mL of s. s. of NaHCO₃ 3.5 ml and stirred for 45 min at 100° C. After this time the reaction was cooled down to rt and extracted with DCM 3 times. The combined organic fractions were dried through a phase separator and concentrate under vacuum. The resulting material was purified 2 times by FC on RP using acidic conditions (eluting from CH₃CN/H₂O 5:95 +0.1% of formic acid to CH₃CN/H₂O 95:5 +0.1% of formic acid) affording the product of formula N-[3-(azepan-1-yl)-4-(4,5-dimethyl-1,2,4-triazol-3-yl)phenyl]cyclopropanecarboxamide.
Yield: 4.8 mg

H NMR (500 MHz, METHANOL-d4) δ ppm 7.65 (d, 1 H), 7.23-7.16 (m, 1 H), 7.16-7.09 (m, 1 H), 3.40 (s, 3 H), 3.09 (t, 4 H), 2.48 (s, 3 H), 1.83-1.73 (m, 1 H), 1.63-1.41 (m, 8 H), 0.97 (quin, 2 H), 0.92-0.82 (m, 2 H); LC-MS: m/z 354.26 (MH+).

Preparation: N-[3-(3,5-dimethylpiperidin-1-yl)-4-(4-methyl-5-sulfanyl-1,2,4-triazol-3-yl)phenyl]cyclopropanecarboxamide

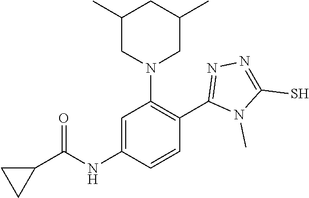 [SMILES: CC1CC(C)CN(c2cc(NC(=O)C3CC3)ccc2-c2nnc(S)n2C)C1]

To a solution of N-[3-(3,5-dimethylpiperidin-1-yl)-4-(hydrazinecarbonyl)phenyl]cyclopropanecarboxamide (1.3 g, 3.93 mmol) in THF (23.36 mL) methylimino(sulfanylidene)methane (0.316 g, 4.33 mmol) was added and the reaction was stirred at 130° C. for 8 minutes under MW irradiation. Then the reaction was cooled down to RT and 30 mL of s. s. of Na HCO₃ was added. The resulting mixture was heated to reflux ON. The day after the reaction was cooled down to RT and acidified with HCI 2N until pH =1. The aqueous solution was then extracted with AcOEt 3 times. The combined organic fractions were washed with brine, dried over Na₂SO₄, filtered and concentrated under vacuum. The residue was purified by FC on silica gel (eluting from 100% of cHex to cHex/AcOEt 7:3) affording the formula compound N-[3-(3,5-dimethylpiperidin-1-yl)-4-(4-methyl-5-sulfanyl-1,2,4-triazol-3-yl)phenyl]cyclopropanecarboxamide.
Yield: 1.1 g
LC-MS: m/z 386.2 (MH+)
   Preparation: N-[4-(4-methyl-5-sulfanyl-1,2,4-triazol-3-yl)-3-pyrrolidin-1-ylphenyl]cyclopropanecarboxamide

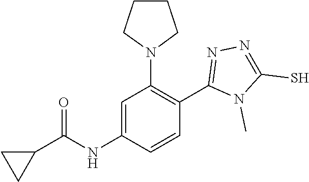 [SMILES: Cn1c(S)nnc1-c1ccc(NC(=O)C2CC2)cc1N1CCCC1]

The synthesis of the title compound was effected analogously to the synthesis of N-[3-(3,5-dimethylpiperidin-1-yl)-4-(4-methyl-5-sulfanyl-1,2,4-triazol-3-yl)phenyl]cyclopropanecarboxamide wherein N-[4-(hydrazinecarbonyl)-3-pyrrolidin-1-ylphenyl]cyclopropanecarboxamide was used instead of N-[3-(3,5-dimethylpiperidin-1-yl)-4-(hydrazinecarbonyl)phenyl]cyclopropanecarboxamide. The title compound was obtained in a yield of 18% (405 mg).

1H NMR (400 MHz, DMSO-d6) δ 13.73 (s, 1H), 10.25 (s, 1H), 7.28 (d, 1H), 7.12 (d, 1H), 7.04 (dd, 1H), 3.21(s, 3H), 2.89 (d, 4H), 1.89-1.71 (m, 5H), 0.88-0.71 (m, 4H); LC-MS: m/z 344.16 (MH+)

Preparation: N-[4-[4-[2-(dimethylamino)ethyl]-5-sulfanyl-1,2,4-triazol-3-yl]-3-(3,5-dimethylpiperidin-1-yl)phenyl]cyclopropanecarboxamide

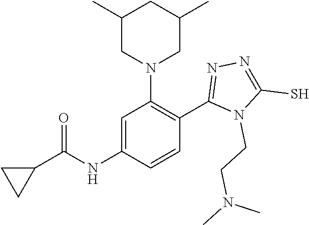 [SMILES: CC1CC(C)CN(c2cc(NC(=O)C3CC3)ccc2-c2nnc(S)n2CCN(C)C)C1]

To a solution of bis(1-imidazolyl)methanethione (177.98 mg, 1 mmol) in THF (7.566 mL) N′,N′-dimethylethane-1,2-diamine (0.09 mL, 0.830 mmol) was added and stirred for 15 min. At this solution N-[3-(3,5-dimethylpiperidin-1-yl)-4-(hydrazinecarbonyl)phenyl]cyclopropanecarboxamide (250.0 mg, 0.760 mmol) was added the resulting solution was heated to 130° C. for 10 minutes under MW irradiation. After this time the reaction was concentrated under vacuum and 10 mL of s. s. of NaHCO₃ were added. The resulting mixture was further stirred at 100° C. for 1 hour under MW irradiation. The reaction mixture was brought to pH 2 with HCI 2N and concentrated under reduced pressure. The row material was purified by FC on RP using acidic conditions (eluting from CH₃CN/H₂O 5:95 +0.1% of formic acid to CH₃CN/H₂O 95:5 +0.1% of formic acid) affording the product of formula N-[4-[4-[2-(dimethylamino)ethyl]-5-sulfanyl-1,2,4-triazol-3-yl]-3-(3,5-dimethylpiperidin-1-yl)phenyl]Cyclopropanecarboxamide.
Yield: 70 mg
LC-MS: 443.5 (MH+).
   Preparation: N-[3-(3,5-dimethylpiperidin-1-yl)-4-(4-methyl-5-phenylsulfanyl-1,2,4-triazol-3-yl)phenyl]cyclopropanecarboxamide

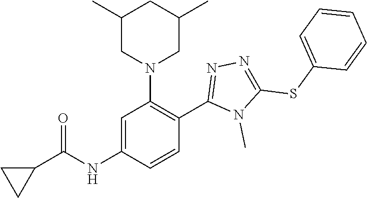 [SMILES: CC1CC(C)CN(c2cc(NC(=O)C3CC3)ccc2-c2nnc(Sc3ccccc3)n2C)C1]

### Example 114a, 114b, 114c

lodobenzene (0.09 mL, 0.780 mmol) was added to a solution of copper (I) iodide (6.21 mg, 0.030 mmol), 1,10-phenanthroline (11.69 mg, 0.060 mmol), potassium carbonate (116.51 mg, 0.840 mmol) in dry DMF (4.323 mL). At this solution a solution of N-[3-(3,5-dimethylpiperidin-1-yl)-4-(4-methyl-5-sulfanyl-1,2,4-triazol-3-yl)phenyl]cyclopropanecarboxamide (250.0 mg, 0.650 mmol) in dry DMF (2.162 mL) was added and the resulting mixture was degassed with Schlenk line technique. The mixture was heated to 120° C. ON. The day after the reaction mixture was diluted with water and extracted with AcOEt 3 times. The combined organic phases were washed with Brine, dried over Na₂SO₄, filtered and concentrated under reduced pressure. The raw material was purified by FC on RP using basic conditions (eluting from 100% of Water +0.1% of NH₄OH to Water +0.1% NH₄OH/CH₃CN 6:4) affording the formula product N-[3-(3,5-dimethylpiperidin-1-yl)-4-(4-methyl-5-phenylsulfanyl-1,2,4-triazol-3-yl)phenyl]Cyclopropanecarboxamide as mixture of syn/anti isomers.
Yield: 70 mg
LC-MS: m/z 462.03 (M +Na)

The mixture of syn/anti isomers (Example 114c) was then separated into single isomers by semi-preparative chiral HPLC

| | Column | Chiralcel OD-H (25 × 2.0 cm), 5 μ | |
|---|---|---|---|
| | Mobile phase % v/v | n-Hexane/Ethanol 60/40% v/v | |
| | Flow rate (ml/min) | 17 | ml/min |
| | DAD detection | 220 | nm |
| Preparative chiral | Loop | 750 | μL |
| chromatography | Total amount | 70 | mg |
| protocol: | Injection | 8.5 | mg (each injection) |

| | |
|---|---|
| 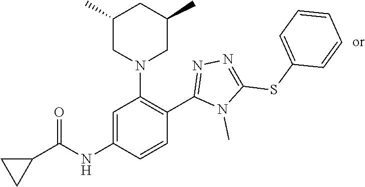 [SMILES: C[C@@H]1C[C@@H](C)CN(c2cc(NC(=O)C3CC3)ccc2-c2nnc(Sc3ccccc3)n2C)C1] | Ant isomer Rt 4.16 min 9.4 mg LC-MS: m/z 362.03 (MH+) 1H NMR (500 MHz, DMSO-d6 ) δ ppm 10.38 (s, 1 H), 7.59 (d, 1 H), 7.44-7.31 (m, 6 H), 7.22 (d, 1 H), 3.34-3.29 (m, 3 H), 2.69 (br dd, 2 H), 2.37 (dd, 2 H), 1.85-1.74 (m, 1 H), 1.71-1.58 (m, 2 H), 1.18 (t, 2 H), 0.86-0.77 (m, 4 H), 0.57 (d, 6 H). Example 114a |
| 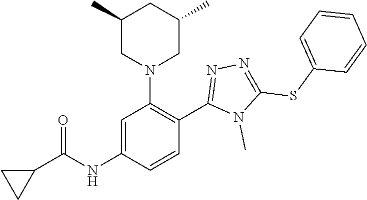 [SMILES: C[C@H]1C[C@H](C)CN(c2cc(NC(=O)C3CC3)ccc2-c2nnc(Sc3ccccc3)n2C)C1] | |
| 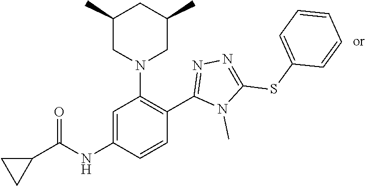 | Syn isomer Rt 6.63 min 57.1 LC-MS: m/z 427.4 (MH+) 1H NMR (500 MHz, DMSO-d6 ) δ ppm 10.37 (s, 1 H), 7.54 (d, 1 H), 7.42-7.34 (m, 5 H), 7.34-7.30 (m, 1 H), 7.29-7.23 (m, 1 H), 3.32 (s, 3 H), 2.82 (br d, 2 H), 1.95 (br t, 2 H), 1.83-1.75 (m, 1 H), 1.49 (br d, 1 H), 1.15-0.98(m, 2 H), 0.84-0.78 (m, 4 H), 0.67 (d, 6 H), 0.44 (q, 1 H), Example 114b |
| 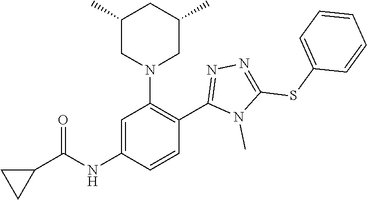 [SMILES: C[C@@H]1C[C@H](C)CN(c2cc(NC(=O)C3CC3)ccc2-c2nnc(Sc3ccccc3)n2C)C1] | |

Preparation: N-[4-[4-[2-(dimethylamino)ethyl]-5-phenylsulfanyl-1,2,4-triazol-3-yl]-3-(3,5-dimethylpiperidin-1-yl)phenyl]cyclopropanecarboxamide

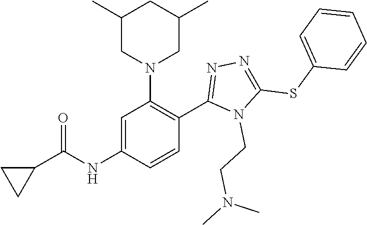 [SMILES: CC1CC(C)CN(c2cc(NC(=O)C3CC3)ccc2-c2nnc(Sc3ccccc3)n2CCN(C)C)C1]

### Example 115

The synthesis of the title compound was effected analogously to the synthesis of the compound of Example 114a, b (N-[3-(3,5-dimethylpiperidin-1-yl)-4-(4-methyl-5-phenylsulfanyl-1,2,4-triazol-3-yl)phenyl]cyclopropanecarboxamide) wherein N-[4-[4-[2-(dimethylamino)ethyl]-5-sulfanyl-1,2,4-triazol-3-yl]-3-(3,5-dimethylpiperidin-1-yl)phenyl]cyclopropanecarboxamide was used instead of N-[3-(3,5-dimethylpiperidin-1-yl)-4-(4-methyl-5-sulfanyl-1,2,4-triazol-3-yl)phenyl]cyclopropanecarboxamide. The title compound was obtained in a yield of 41% (35 mg) as a mixture of syn/anti isomers.

1H NMR (500 MHz, DMSO-d6) δ ppm 10.37 (s, 1 H), 7.53 (d, 1 H), 7.40-7.30 (m, 6 H), 7.24 (d, 1 H), 3.99 (t, 2 H), 3.00-2.75 (m, 2 H), 2.07 (t, 2 H), 2.04-1.86 (m, 2 H), 1.82 (s, 6 H), 1.80-1.75 (m, 1 H), 1.52 (br d, 1 H), 1.35-0.95(m, 2 H), 0.84-0.79 (m, 4 H), 0.77-0.54 (m, 6 H), 0.47 (q, 1 H); LC-MS: m/z 519.3 (MH+)

Prearation: N-[4-(4-methyl-5-pyridin-2-ylsulfanyl-1,2,4-triazol-3-yl)-3-pyrrolidin-1-ylphenyl]cyclopropanecarboxamide

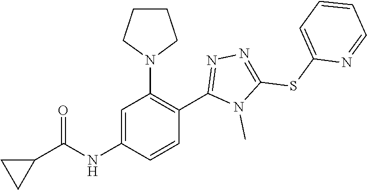 [SMILES: Cn1c(Sc2ccccn2)nnc1-c1ccc(NC(=O)C2CC2)cc1N1CCCC1]

### Example 116

The synthesis of the title compound was effected analogously to the synthesis of the compound of Example 114a, b (N-[3-(3,5-dimethylpiperidin-1-yl)-4-(4-methyl-5-phenylsulfanyl-1,2,4-triazol-3-yl)phenyl]cyclopropanecarboxamide) wherein N-[4-(4-methyl-5-sulfanyl-1,2,4-triazol-3-yl)-3-pyrrolidin-1-ylphenyl]cyclopropanecarboxamide and 2-iodopyridine were used instead of N-[3-(3,5-dimethylpiperidin-1-yl)-4-(4-methyl-5-sulfanyl-1,2,4-triazol-3-yl)phenyl]cyclopropanecarboxamide and iodobenzene respectively. The title compound was obtained in a yield of 27% (43 mg).

1H NMR (400 MHz, DMSO-d6) δ 10.25 (s, 1H), 8.35 (ddd, 1H), 7.79 (ddd, 1H), 7.34 (dt, 1H), 7.31 (d, 1H), 7.27 (ddd, 1H), 7.14 (d, 1H), 7.06 (dd, 1H), 3.29 (s, 3H), 2.93-2.84 (m, 4H), 1.84 -1.77 (m, 5H), 0.87-0.75 (m, 4H); LC-MS: m/z 421.15 (MH+).

Preparation: N-[4-(4-methyl-5-pyridin-3-ylsulfanyl-1,2,4-triazol-3-yl)-3-pyrrolidin-1-ylphenyl]cyclopropanecarboxamide

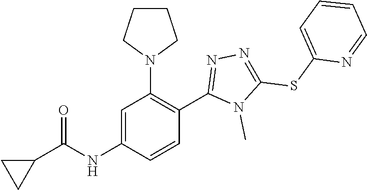 [SMILES: Cn1c(Sc2ccccn2)nnc1-c1ccc(NC(=O)C2CC2)cc1N1CCCC1]

### Example 117

The synthesis of the title compound was effected analogously to the synthesis of the compound of Example 114a, b (N-[3-(3,5-dimethylpiperidin-1-yl)-4-(4-methyl-5-phenylsulfanyl-1,2,4-triazol-3-yl)phenyl]cyclopropanecarboxamide) wherein N-[4-(4-methyl-5-sulfanyl-1,2,4-triazol-3-yl)-3-pyrrolidin-1-ylphenyl]cyclopropanecarboxamide and 3-iodopyridine were used instead of N-[3-(3,5-dimethylpiperidin-1-yl)-4-(4-methyl-5-sulfanyl-1,2,4-triazol-3-yl)phenyl]cyclopropanecarboxamide and iodobenzene respectively. The title compound was obtained in a yield of 88% (140 mg).

1H NMR (400 MHz, DMSO-d6) δ 10.25 (s, 1H), 8.57 (dd, 1H), 8.54 (dd, 1H), 7.78 (ddd, 1H), 7.44 (ddd, 1H), 7.28 (d, 1H), 7.13 (d, 1H), 7.06 (dd, 1H), 3.32 (s, 3H), 2.78-2.65 (m, 4H), 1.80 (tt, 1H), 1.77-1.61 (m, 4H), 0.87-0.73 (m, 4H); LC-MS: m/z 421.17 (MH+).

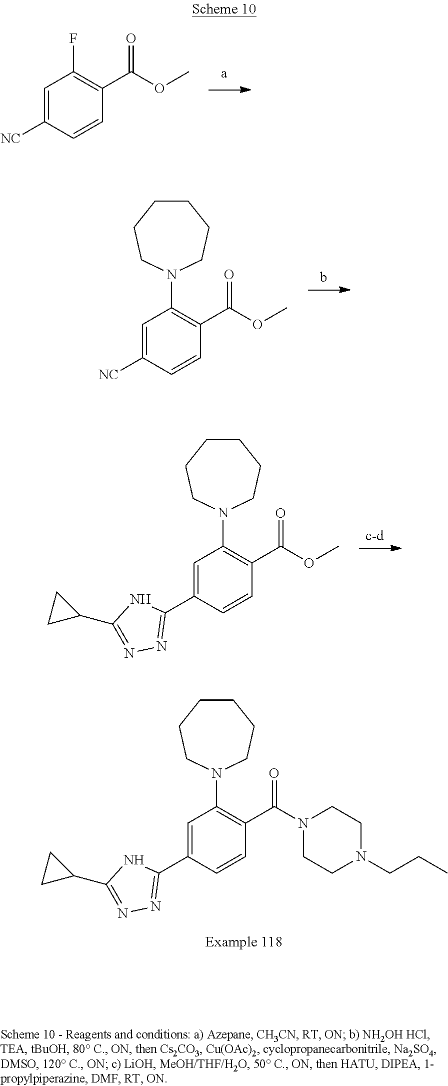

Preparation: methyl 2-(azepan-1-yl)-4-cyanobenzoate

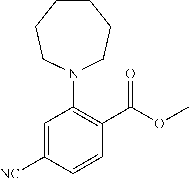 [SMILES: COC(=O)c1ccc(C#N)cc1N1CCCCCC1]

A mixture of methyl 4-cyano-2-fluorobenzoate (1.2 g, 6.7 mmol) and Homopiperidine (3.77 mL, 33.49 mmol) in MeCN (11 mL) was stirred at RT overnight. The reaction was concentrated under vacuum and the residue was purified by FC on NH column (eluting from 100% of cHex to cHex/AcOEt 9:1) affording the product of formula methyl 2-(azepan-1-yl)-4-cyanobenzoate.
Yield: 1.3 g ¹ NMR (400 MHz, Chloroform-d) δ 7.56 (d, 1H), 7.19 (d, 1H), 6.97 (dd, 1H), 3.92 (s, 3H), 3.40-3.23 (m, 4H), 1.86-1.77 (m, 4H), 1.62 (dt, 4H); LC-MS: m/z 258.8 (MH+).
   Preparation: methyl 2-(azepan-1-yl)-4-(3-cyclopropyl-1H-1,2,4-triazol-5-yl)benzoate

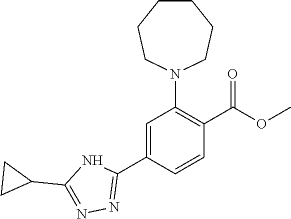 [SMILES: COC(=O)c1ccc(-c2nnc(C3CC3)[nH]2)cc1N1CCCCCC1]

A mixture of methyl 2-(azepan-1-yl)-4-cyanobenzoate (317.65 mg, 1.23 mmol), hydroxylamine hydrochloride (94.0 mg, 1.35 mmol) and triethylamine (0.34 mL, 2.46 mmol) in tert-butanol (3.5 mL) was stirred 80° C. ON. After this time the reaction was cooled down to RT and cyclopropanecarbonitrile (165.0 mg, 2.46 mmol), copper diacetate (45.17 mg, 0.250 mmol), dicesium carbonate (1.21 g, 3.69 mmol) and anhydrous Na₂SO₄ (1.2 g, 8.61 mmol) and DMSO (3 mL) were added and the reaction was stirred at 120° C. overnight. The reaction was cooled down to RT and concentrated. The residue was purified by FC on NH column (eluting from 100% of cHex to cHex/AcOEt 70:30) affording the product of formula methyl 2-(azepan-1-yl)-4-(3-cyclopropyl-1H-1,2,4-triazol-5-yObenzoate.
Yield: 30 mg
LC-MS: m/z 341.2 (MH+)
   Preparation: [2-(azepan-1-yl)-4-(5-cyclopropyl-4H-1,2,4-triazol-3-ypphenyl]-(4-propylpiperazin-1-yl)methanone

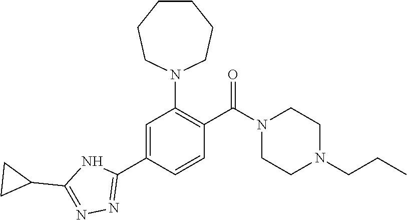 [SMILES: CCCN1CCN(C(=O)c2ccc(-c3nnc(C4CC4)[nH]3)cc2N2CCCCCC2)CC1]

### Example 118

To a solution of methyl 2-(azepan-1-yl)-4-(5-cyclopropyl-4H-1,2,4-triazol-3-yl)benzoate (30.0 mg, 0.090 mmol) in THF (0.111 mL) and Methanol (0.037 mL) a solution of lithium hydroxide (4.22 mg, 0.180 mmol) in Water (0.037 mL) was added. The reaction was stirred at 50° C. overnight. The day after the reaction was cooled down to RT, acidified with HCI 1 N until pH =5 and concentrated under vacuum affording the intermediate of formula 2-(azepan-1-yl)-4-(5-cyclopropyl-4H-1,2,4-triazol-3-yl)benzoic acid, which was used in the next step without further purification. LC-MS: m/z 327.1 (MH+). The intermediate 2-(azepan-1-yl)-4-(5-cyclopropyl-4H-1,2,4-triazol-3-yl)benzoic acid (25.0 mg, 0.075 mmol) was dissolved in DMF (0.5 mL) and then N,N-Diisopropylethylamine (0.05 mL, 0.310 mmol) and HATU: [dimethylamino(3-triazolo[4,5-b]pyridinyloxy)methylidene]-dimethylammonium hexafluorophosphate (69.9 mg, 0.180 mmol) were added. The mixture was stirred at RT for 10 min and then a solution of 1-propylpiperazine (51.1 mg, 0.180 mmol) in DMF (0.5 mL) was added dropwise. The reaction mixture was stirred at RT for 1hrs. The reaction mixture was purified by SCX first washing with MeOH and then with NH₃ 1M in MeOH. Basic fractions were evaporated and the residue was purified by FC on NH column (eluting from 100% of AcOEt to AcOEt/MeOH 9:1). The product was further purified by FC on RP using acidic conditions (eluting from CH₃CN/H₂O 5:95 +0.1% of formic acid to CH₃CN/H₂O 95:5 +0.1% of formic acid) affording the product of formula [2-(azepan-1-yl)-4-(5-cyclopropyl-4H-1,2,4-triazol-3-yl)phenyl]-(4-propylpiperazin-1-yl)methanone.
Yield: 15 mg

1H NMR (500 MHz, DMSO-d6) δ ppm 14.26 (m, 1 H) - 13.34, 7.54 (br s, 1 H), 7.38 (br d, 1 H), 7.08 (br d, 7.3 Hz, 1 H), 3.26 (t, 4 H), 3.55-3.10 (m, 2 H), 3.75-3.10 (m, 2 H), 2.27-2.21 (m, 2 H), 2.48-2.13 (m, 4 H), 2.06 (br s, 1 H), 1.77-1.48 (m, 8 H), 1.42 (sxt, 2 H), 1.10-0.87 (m, 4 H), 0.84 (t, 3 H); LC-MS: m/z 437.4 (MH+).

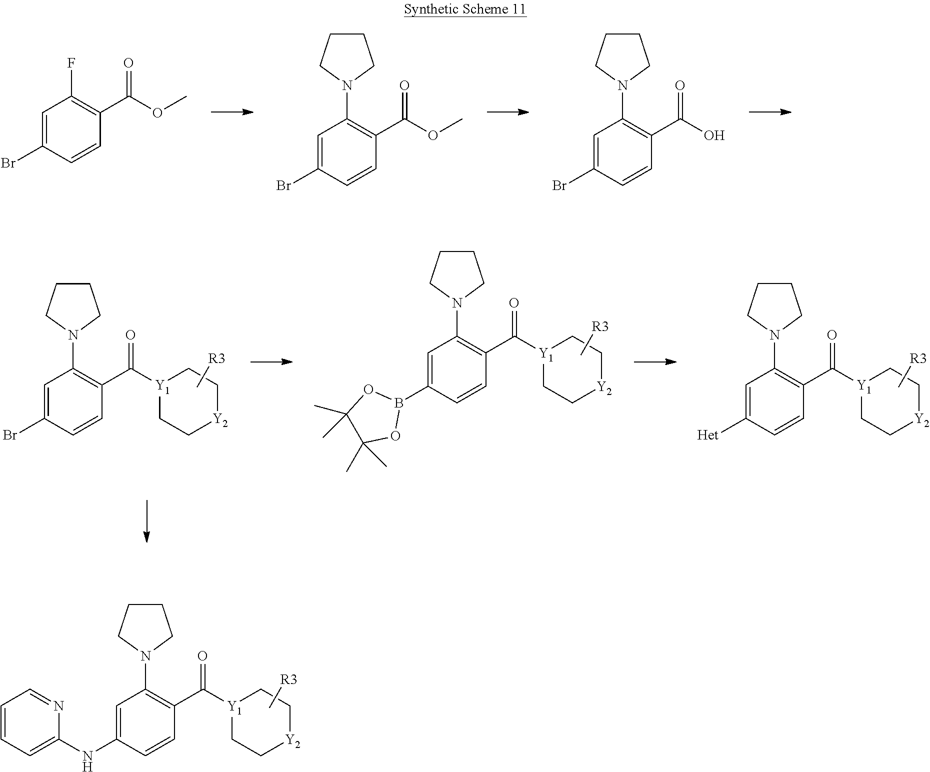

Preparation: methyl 4-bromo-2-pyrrolidin-1-ylbenzoate

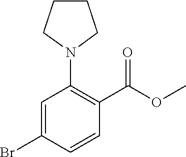 [SMILES: COC(=O)c1ccc(Br)cc1N1CCCC1]

To a solution of methyl 4-bromo-2-fluorobenzoate (1.0 g, 4.29 mmol) in DMF (7.122 mL) pyrrolidine (1.43 mL, 17.17 mmol) was added and the reaction was stirred at 80° C. for 8 h. After this time the reaction was cooled down to RT and H₂O was added. The mixture was stirred for 10 min and filtered. The obtained solid was washed with H2O 2O and dried under vacuum affording the product of formula methyl 4-bromo-2-pyrrolidin-1-ylbenzoate,
Yield: 1 g

1H NMR (400 MHz, DMSO-d6) δ 7.35 (d, 1H), 6.92 (d, 1H), 6.82 (dd, 1H), 3.79 (s, 3H), 3.19-3.12 (m, 4H), 1.92-1.84 (m, 4H); LC-MS: m/z 284.03, 286.01 (MH+).

Preparation: 4-bromo-2-pyrrolidin-1-ylbenzoic acid

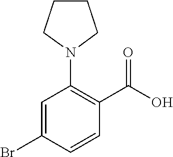 [SMILES: O=C(O)c1ccc(Br)cc1N1CCCC1]

To a mixture of methyl 4-bromo-2-pyrrolidin-1-ylbenzoate (0.5 g, 1.76 mmol) in THF (3 mL)/Methanol (1 mL) a solution of lithium hydroxide hydrate (147.67 mg, 3.52 mmol) in Water (1 mL) was added and the reaction was stirred at 50° C. ON. The day after HCI 3 N was added until pH =1 and the reaction was extracted with AcOEt. The organic phase was then washed with Brine, dried over Na₂SO₄, filtered and concentrated under vacuum affording the product of formula 4-bromo-2-pyrrolidin-1-ylbenzoic acidm which was used in the next step without further purification.
Yield: 387 mg

1H NMR (400 MHz, DMSO-d6) δ 12.81 (s, 1H), 7.38 (dd, 1H), 6.91 (t, 1H), 6.81 (dt, 1H), 3.17 (q, 4H), 1.88 (h, 4H); LC-MS: m/z 269.99, 271.97 (MH+).

Preparation: (4-bromo-2-pyrrolidin-1-ylphenyl)-(4-propylpiperazin-1-yl)methanone

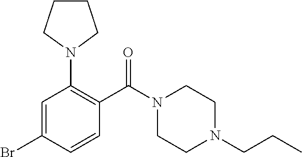 [SMILES: CCCN1CCN(C(=O)c2ccc(Br)cc2N2CCCC2)CC1]

4-bromo-2-pyrrolidin-1-ylbenzoic acid (389.0 mg, 1.44 mmol), HATU: [dimethylamino(3-triazolo[4,5-b]pyridinyloxy)methylidene]-dimethylammonium hexafluorophosphate (657.08 mg, 1.73 mmol) and N,N-Diisopropylethylamine (0.5 mL, 2.88 mmol) were mixed in DMF (6 mL) and stirred for 5 min, then a solution of 1-propylpiperazine dihydrobromide (480.34 mg, 1.66 mmol) and N,N-Diisopropylethylamine (0.5 mL, 2.88 mmol) in DMF (2 mL) was added. The reaction mixture was stirred at RT overnight. The day after the solvent was removed under vacuum. The residue was purified by SCX cartridge first washing with MeOH and then eluting with NH₃ 1M in MeOH. The basic fractions were evaporated and the residue was purified by FC on NH column (eluting from 100% of cHex to cHex/AcOEt 7:3) affording the product of formula (4-bromo-2-pyrrolidin-1-ylphenyl)-(4-propylpiperazin-1-yl)methanone.
Yield: 430 mg

1H NMR (400 MHz, DMSO-d6) δ 6.94-6.85 (m, 1H), 6.84-6.73 (m, 2H), 3.72-3.62 (m, 1H), 3.49 (ddd, 1H), 3.26-3.02 (m, 6H), 2.44-2.16 (m, 6H), 1.92-1.80 (m, 4H), 1.42 (h, 2H), 0.84 (t, 3H); LC-MS: m/z 380.10, 382.10 (MH+).

Preparation: (4-propylpiperazin-1-yl)-[4-(pyridin-2-ylamino)-2-pyrrolidin-1-ylphenyl]methanone

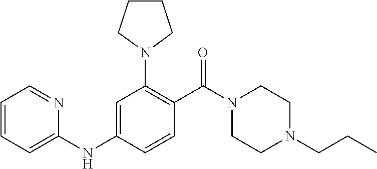 [SMILES: CCCN1CCN(C(=O)c2ccc(Nc3ccccn3)cc2N2CCCC2)CC1]

### Example 119

A mixture of (4-bromo-2-pyrrolidin-1-ylphenyl)-(4-propylpiperazin-1-yl)methanone (0.2 g, 0.530 mmol), (1E,4E)-1,5-diphenyl-3-penta-1,4-dienone;palladium (24.08 mg, 0.030 mmol), dicesium carbonate (257.01 mg, 0.790 mmol), 2-pyridinamine (59.39 mg, 0.630 mmol) and [1-(2-diphenylphosphino-1-naphthalenyl)-2-naphthalenyl]-diphenylphosphine (32.74 mg, 0.050 mmol) were suspended in Toluene (2.815 mL). The reaction was degassed with N₂ for 10 min and then stirred at 100° C. ON. The day after the reaction was filtered through a pad of celite and concentrated under vacuum. The residue was purified by FC on RP using basic conditions (eluting from H₂O +0.1% of ammonium hydroxide/ CH₃CN 95:5 to H₂O +0.1% of ammonium hydroxide/ CH₃CN 5:95) affording the product of formula (4-propylpiperazin-1-yl)-[4-(pyridin-2-ylamino)-2-pyrrolidin-1-ylphenyl]methanone.
Yield: 5.8 mg

¹ NMR (400 MHz, DMSO-d₆) δ 8.95 (s, 1H), 8.14 (d, 1H), 7.54 (t, 1H), 7.15-6.93 (m, 2H), 6.85 (dd, 2H), 6.79-6.69 (m, 1H), 3.53 (s, 4H), 3.27-2.97 (m, 6H), 2.24 (t, 4H), 1.88 (d, 4H), 1.43 (q, 2H), 0.85 (t, 3H);
LC-MS: m/z 394.3 (MH+).
   Preparation: (4-bromo-2-pyrrolidin-1-ylphenyI)-(4-methyl-2-phenylpiperazin-1-yl)methanone

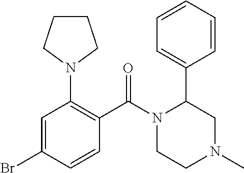 [SMILES: CN1CCN(C(=O)c2ccc(Br)cc2N2CCCC2)C(c2ccccc2)C1]

The synthesis of the title compound was effected analogously to the synthesis of (4-bromo-2-pyrrolidin-1-ylphenyl)-(4-propylpiperazin-1-yl)methanone wherein 1-methyl-3-phenylpiperazine was used instead of 1-propylpiperazine dihydrobromide. The title compound was obtained in a yield of 66% (1.04 g).

¹ NMR (400 MHz, DMSO-d₆) δ 7.83-7.15 (m, 5H), 7.09-6.67 (m, 3H), 5.85-4.30 (m, 1H), 3.31-3.21 (m, 2H), 3.06 (d, 2H), 2.81— 2.64 (m, 2H), 2.45-2.11 (m, 5H), 2.07-1.80 (m, 4H), 1.75-1.54 (m, 2H), LC-MS: m/z 428.08, 430.10 (MH+).

Preparation: (4-methyl-2-phenylpiperazin-1-yl)-[2-pyrrolidin-1-yl-4-(4,4,5,5-tetramethyl-1,3,2-dioxaborolan-2-yl)phenyl]methanone

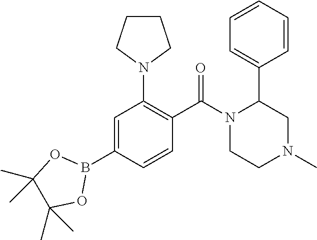

### Example 120

A mixture of 4,4,5,5-tetramethyl-2-(4,4,5,5-tetramethyl-1,3,2-dioxaborolan-2-yl)-1,3,2-dioxaborolane (311.22 mg, 1.23 mmol), (4-bromo-2-pyrrolidin-1-ylphenyl)-(4-methyl-2-phenylpiperazin-1-yl)methanone (500.0 mg, 1.17 mmol) and potassium acetate (347.19 mg, 3.5 mmol) in 1,4-Dioxane (22 mL) was degassed by 5 cycles of nitrogen/vacuum. Then [1,1′-Bis(diphenylphosphino)ferrocene]dichloropalladium(II), complex with dichloromethane (19.11 mg, 0.020 mmol) was added and after degassing again by 5 cycles of nitrogen/vacuum, the reaction mixture was heated at 100° C. for 2 hours. After this time the reaction mixture was cooled down to RT and filtered through a pad of celite washing with AcOEt. The organic solution was concentrate under reduced pressure affording the product of formula (4-methyl-2-phenylpiperazin-1-yl)-[2-pyrrolidin-1-yl-4-(4,4,5,5-tetramethyl-1,3,2-dioxaborolan-2-yl)phenyl]Methanone, which was used in the next step without further purification.
Yield: 559 mg
LC-MS: m/z 476.22 (MH+).
   Preparation: [4-(1-methylimidazol-2-yl)-2-pyrrolidin-1-ylphenyl]-(4-methyl-2-phenylpiperazin-1-yl)methanone

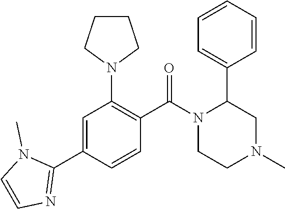 [SMILES: CN1CCN(C(=O)c2ccc(-c3nccn3C)cc2N2CCCC2)C(c2ccccc2)C1]

### Example 121

A mixture of palladium tetrakis triphenylphosphine (6.08 mg, 0.010 mmol), aqueous solution of sodium carbonate 2M (0.16 mL, 0.320 mmol) and (4-methyl-2-phenylpiperazin-1-yl)-[2-pyrrolidin-1-yl-4-(4,4,5,5-tetramethyl-1,3,2-dioxaborolan-2-yl)phenyl]methanone (50.0 mg, 0.110 mmol) in 1,2-dimethoxyethane (1 mL) was degassed by 5 cycles of nitrogen/vacuum. Then 2-bromo-1-methylimidazole (0.01 mL, 0.120 mmol) was added and the vessel was sealed. The reaction mixture was degassed again by 5 cycles of nitrogen/vacuum and then was heated to 95° C. ON. The day after the reaction was cooled down to RT and the mixture was directly purified by FC on RP using acid conditions (eluting from CH₃CN/H₂O 5:95 +0.1% of formic acid to CH₃CN/H₂O 5:5 +0.1% of formic acid). Opportune fractions were collected together and concentrated under vacuum. The residue was further purified by FC on RP using basic conditions (eluting from H₂O +0.1% of ammonium hydroxide/ CH₃CN 95:5 to H₂O +0.1% of ammonium hydroxide/CH₃CN 2:8) affording the product of formula [4-(1-methylimidazol-2-yl)-2-pyrrolidin-1-ylphenyl]-(4-methyl-2-phenylpiperazin-1-yl)methanone as a racemic mixture.
Yield: 11.6 mg

1H NMR (400 MHz, DMSO-d6) δ 7.81-7.69 (m, 1H), 7.49 (d, 1H), 7.44-6.89 (m, 8H), 5.90-4.74 (m, 1H), 3.83-3.68 (m, 3H), 3.56-2.62 (m, 8H), 2.47-2.31 (m, 1H), 2.30-2.15 (m, 3H), 2.10-1.89 (m, 3H), 1.78-1.61 (m, 2H); LC-MS: m/z 430.22 (MH+)

Preparation: [4-(2-methyl-1H-imidazol-5-yl)-2-pyrrolidin-1-ylphenyl]-(4-methyl-2-phenylpiperazin-1-yl)methanone

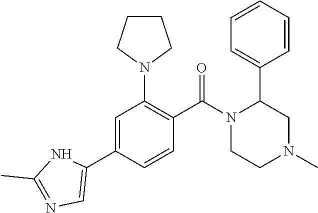

### Example 122

The synthesis of the title compound was effected analogously to the synthesis of the compound of Example 121 ([4-(1-methylimidazol-2-yl)-2-pyrrolidin-1-ylphenyl]-(4-methyl-2-phenylpiperazin-1-yl)methanone) wherein 4-bromo-2-methylimidazole was used instead of 2-bromo-1-methylimidazole. The title compound was obtained in a yield of 38.3% (17.3 mg).

1H NMR (400 MHz, DMSO-d6) δ 11.94 (d, 1H), 8.08-6.54 (m, 9H), 5.89-4.68 (m, 1H), 3.55-2.59 (m, 8H), 2.42-2.11 (m, 7H), 1.95 (s, 3H), 1.77-1.59 (m, 2H); LC-MS: 430.20 (MH+).

Preparation: [4-(5-methyl-1,3,4-oxadiazol-2-yl)-2-pyrrolidin-1-ylphenyl]-(4-methyl-2-phenylpiperazin-1-yl)methanone

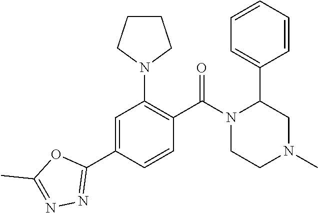

### Example 123

The synthesis of the title compound was effected analogously to the synthesis of the compound of Example 121 ([4-(1-methylimidazol-2-yl)-2-pyrrolidin-1-ylphenyl]-(4-methyl-2-phenylpiperazin-1-yl)methanone) wherein 2-bromo-5-methyl-1,3,4-oxadiazole was used instead of 2-bromo-1-methylimidazole. The title compound was obtained in a yield of 16.5% (7.5 mg) as a racemic mixture.

1H NMR (400 MHz, DMSO-d6) δ 8.03-6.97 (m, 8H), 5.86-4.62 (m, 1H),3.53-3.26 (m, 2H), 3.30-2.54 (m, 9H), 2.37-1.87 (m, 8H), 1.79-1.60 (m, 1H); LC-MS: m/z 432.26 (MH+).

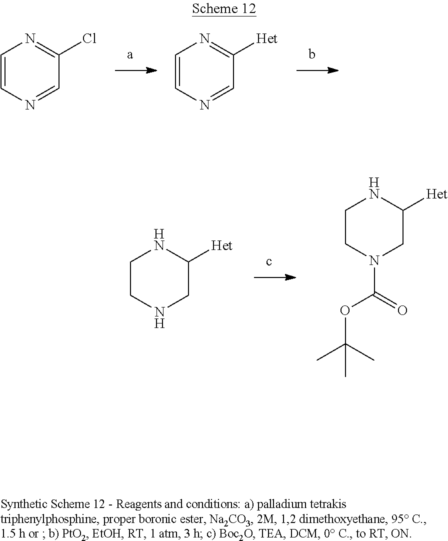

Preparation: 2-thiophen-2-ylpyrazine

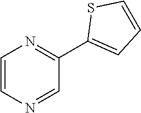 [SMILES: c1csc(-c2cnccn2)c1]

A mixture of 2-chloropyrazine (2.6 mL, 29.13 mmol), thiophen-2-ylboronic acid (4.1 g, 32.04 mmol) and aqueous solution 2 M of sodium carbonate (43.69 mL, 87.38 mmol) in 1,2-dimethoxyethane (100 mL) was degassed by 5 cycles of nitrogen/vacuum. Then palladium tetrakis triphenylphosphine (1.7 g, 1.46 mmol) was added and the vessel was sealed. The reaction mixture was degassed again by 5 cycles of nitrogen/vacuum and then heated to 95° C. ON. The reaction was cooled down to RT, diluted with H₂O and the reaction was extracted with EtOAc (x3). The organic portions were collected, washed with Brine, dried over Na₂SO₄ and the solvent was evaporated under reduced pressure. The residue was purified by FC on NH column (eluting from 100% of cHex to cHex/AcOEt 8:2) affording 2-thiophen-2-ylpyrazine.
Yield: 3.9 g
LC-MS: m/z 162.85 (MH+).
   Preparation: 3-thiophen-2-ylpyrazine

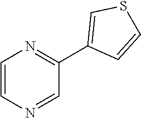 [SMILES: c1cnc(-c2ccsc2)cn1]

The synthesis of the title compound was effected analogously to the synthesis of 2-thiophen-2-ylpyrazine wherein 3-thiophenylboronic acid was used instead of thiophen-2-ylboronic acid. The title compound was obtained in a yield of 85% (3 g).

1H NMR (400 MHz, CHLOROFORM-d) δ 8.94 (d, 1 H) 8.57 (dd, 1 H) 8.45 (d, 1 H) 8.01 (dd, 1 H) 7.71 (dd, 1 H) 7.46 (dd, 1 H); LC-MS: m/z 162.9 (MH+)

Preparation: 2-(1-methylpyrazol-4-yl)pyrazine

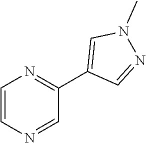

The synthesis of the title compound was effected analogously to the synthesis of 2-thiophen-2-ylpyrazine wherein 1-Methylpyrazole-4-boronic acid pinacol ester was used instead of thiophen-2-ylboronic acid. The title compound was obtained in a yield of 96% (0.8 g).

1H NMR (400 MHz, DMSO-d6) δ 8.97 (d, 1H), 8.55 (dd, 1H), 8.41 (s, 1H), 8.40 (s, 1H), 8.10 (d, 1H), 3.91 (s, 3H); LC-MS: m/z 160.95 (MH+).

Preparation: 2-pyridin-2-ylpyrazine

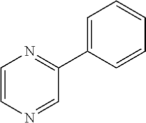 [SMILES: c1ccc(-c2cnccn2)cc1]

A mixture of 2-chloropyrazine (250.0 mg, 2.18 mmol) and tributyl(2-pyridinyl)stannane (0.85 mL, 2.62 mmol) in dry Toluene (10 mL), the solution was degassed and palladium tetrakis triphenylphosphine (126.12 mg, 0.110 mmol) added under nitrogen flux. The suspension was degassed again and stirred ON at 115° C. The day after the reaction mixture was diluted with an aqueous solution of KF 10 mol% and extracted with EtOAc 3 times. The organic fractions were washed with water, Brine, dried over Na₂SO₄, filtered and the solvent removed under reduced pressure. The resulting material was purified by FC on silica gel (eluting from 100% of cHex to cHex/AcOEt 60:40) affording the product of formula 2-pyridin-2-ylpyrazine
Yield: 350 mg

¹ NMR (400 MHz, Chloroform-d) δ 9.66 (d, 1H), 8.75 (ddd, 1H), 8.66-8.61 (m, 2H), 8.39 (dt, 1H), 7.87 (td, 1H), 7.43-7.38 (m, 1H); LC-MS: m/z 158.1 (MH+)

Preparation: 2-thiophen-2-ylpiperazine

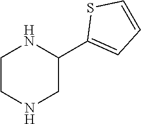 [SMILES: c1csc(C2CNCCN2)c1]

A mixture of 2-thiophen-2-ylpyrazine (275.0 mg, 1.7 mmol) and Platinum(IV) oxide (192.49 mg, 0.850 mmol) in Ethanol (5 mL) and AcOH (1 mL) was stirred under H₂ atmosphere at RT, at 1 atm, for 3 h. The day after the reaction was filtered through a pad of celite and concentrated under vacuum. The residue was purified by SCX first washing with MeOH and then with NH₃ 1M in MeOH affording the product of formula 2-thiophen-2-ylpiperazine.
Yield: 240 mg
LC-MS: m/z 169.0 (MH+).
   Preparation: 2-(1-methylpyrazol-4-yl)piperazine

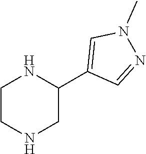

The synthesis of the title compound was effected analogously to the synthesis of 2-thiophen-2-ylpiperazine wherein 2-(1-methylpyrazol-4-yl)pyrazine was used instead of 2-thiophen-2-ylpyrazine. The title compound was obtained in a yield of 53% (550 mg). LC-MS: m/z 167.0 (MH+)

Preparation: 2-thiophen-3-ylpiperazine

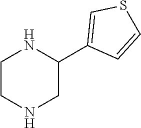 [SMILES: c1cc(C2CNCCN2)cs1]

A mixture of 2-thiophen-3-ylpyrazine (315.0 mg, 1.94 mmol) and Platinum(IV) oxide (220.49 mg, 0.970 mmol) in Ethanol (8 mL) and AcOH (1 mL) was stirred under H2 atmosphere at 6 bar at RT ON. The reaction was filtered through a pad of celite and concentrated under vacuum. The residue was purified by SCX first washing with MeOH and then with NH3 1M in MeOH affording the product of formula 2-thiophen-3-ylpiperazine.
Yield: 150 mg
LC-MS: m/z 169.0 (MH+)
   Preparation: 2-pyridin-2-ylpiperazine

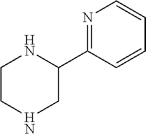 [SMILES: c1ccc(C2CNCCN2)nc1]

The synthesis of the title compound was effected analogously to the synthesis of 2-thiophen-3-ylpiperazine wherein 2-pyridin-2-ylpyrazine was used instead of 2-thiophen-3-ylpyrazine. The title compound was obtained in a yield of 83% (130 mg).

¹ NMR (400 MHz, Chloroform-d) δ 8.57 (ddd, 1H), 7.67 (td, 1H), 7.37 (dt, 1H), 7.19 (ddd, 1H), 3.93 (dd, 1H), 3.28-3.13 (m, 2H), 3.04-2.96 (m, 2H), 2.91— 2.75 (m, 2H).

Preparation: tert-butyl 3-thiophen-2-ylpiperazine-1-carboxylate

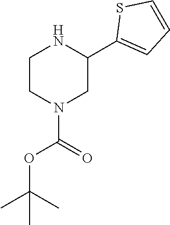 [SMILES: CC(C)(C)OC(=O)N1CCNC(c2cccs2)C1]

A mixture of 2-thiophen-2-ylpiperazine (140.0 mg, 0.830 mmol), Di-tert-butyl dicarbonate (181.59 mg, 0.830 mmol) and triethylamine (0.13 mL, 0.920 mmol) in DCM (4 mL) was stirred at room temperature for 3 h. The reaction was diluted with DCM and washed with Brine. The organic phase was dried over Na₂SO₄,filtered and concentrated under vacuum. The residue was purified by FC on NH column (eluting from 100% of cHex to cHex/AcOEt 90:10) affording the product of formula tert-butyl 3-thiophen-2-ylpiperazine-1-carboxylate.
Yield: 200 mg

1H NMR (400 MHz, CHLOROFORM-d) d pμm 7.21-7.15 (m, 1 H), 7.04-6.99 (m, 2 H), 4.05-3.92 (m., 2 H), 2.90 (dd, 4 H), 1.82-1.57 (m, 2 H), 1.48 (s, 9 H).

Preparation: tert-butyl 3-thiophen-3-ylpiperazine-1-carboxylate

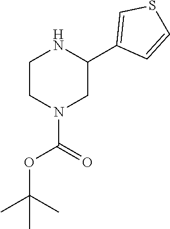 [SMILES: CC(C)(C)OC(=O)N1CCNC(c2ccsc2)C1]

The synthesis of the title compound was effected analogously to the synthesis of tert-butyl 3-thiophen-2-ylpiperazine-1-carboxylate wherein 2-thiophen-3-ylpiperazine was used instead of 2-thiophen-2-ylpiperazine. The title compound was obtained in a yield of 20% (45 mg).
LC-MS: m/z 269.1 (MH+).
   Preparation: tert-butyl 3-(1-methylpyrazol-4-yl)piperazine-1-carboxylate

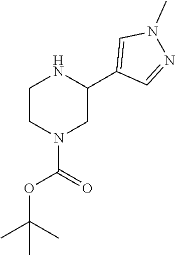 [SMILES: Cn1cc(C2CN(C(=O)OC(C)(C)C)CCN2)cn1]

The synthesis of the title compound was effected analogously to the synthesis of tert-butyl 3-thiophen-2-ylpiperazine-1-carboxylate wherein 2-(1-methylpyrazol-4-yl)piperazine was used instead of 2-thiophen ylpiperazine. The title compound was obtained in a yield of 99% (430 mg).
LC-MS: m/z 267.1 (MH+)
   Preparation: tert-butyl 3-pyridin-2-ylpiperazine-1-carboxylate

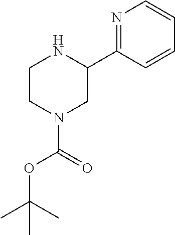 [SMILES: CC(C)(C)OC(=O)N1CCNC(c2ccccn2)C1]

The synthesis of the title compound was effected analogously to the synthesis of tert-butyl 3-thiophen-2-ylpiperazine-1-carboxylate wherein 2-pyridin-2-ylpiperazine was used instead of 2-thiophen-2-ylpiperazine. The title compound was obtained in a yield of 46% (185 mg).
LC-MS: m/z 264.3 (MH+)
   Preparation: tert-butyl 3-(3-fluorophenyl)piperazine-1-carboxylate

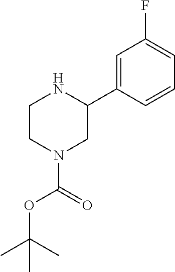 [SMILES: CC(C)(C)OC(=O)N1CCNC(c2cccc(F)c2)C1]

To a solution of 2-(3-fluorophenyl)piperazine (200.0 mg, 1.11 mmol) and triethylamine (0.17 mL, 1.22 mmol) in DCM (2.5 mL) a solution of Di-tert-butyl dicarbonate (242.2 mg, 1.11 mmol) in DCM (1 mL) was slowly added and the reaction was stirred at RT for 1 h. The solution was diluted with other DCM and washed with H₂O. The organic phase was dried over Na₂SO₄, filtered and concentrated under vacuum affording the product of formula tert-butyl 3-(3-fluorophenyl)piperazine-1-carboxylate as a racemic mixture. The product was used in the next step without further purification.
Yield: 300 mg

¹ NMR (400 MHz, Chloroform-d) δ 7.36-7.30 (m, 1H), 7.24-7.11 (m, 2H), 7.03-6.93 (m, 1H), 4.06 (s,

2H), 3.73 (dd, 1H), 3.18-3.04 (m, 1H), 3.00-2.88 (m, 2H), 2.72 (s, 1H), 1.83 (s, 1H), 1.49 (s, 9H); LC-MS: m/z 281.1 (MH+)

Preparation: tert-butyl 3-(2-fluorophenyl)piperazine-1-carboxylate

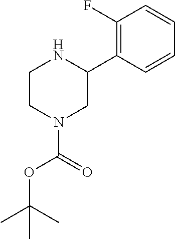 [SMILES: CC(C)(C)OC(=O)N1CCNC(c2ccccc2F)C1]

The synthesis of the title compound was effected analogously to the synthesis of tert-butyl 3-(3-fluorophenyl)piperazine-1-carboxylate wherein 2-(2-fluorophenyl)piperazine-1,4-diium dichloride was used instead of 2-(3-fluorophenyl)piperazine. The title compound was obtained in a yield of 99% (220 mg).
LC-MS: m/z 281.1 (MH+).
   Preparation: tert-butyl 3-(4-fluorophenyl)piperazine-1-carboxylate

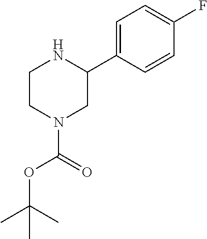 [SMILES: CC(C)(C)OC(=O)N1CCNC(c2ccc(F)cc2)C1]

The synthesis of the title compound was effected analogously to the synthesis of tert-butyl 3-(3-fluorophenyl)piperazine-1-carboxylate wherein 2-(4-fluorophenyl)piperazine was used instead of 2-(3-fluorophenyl)piperazine. The title compound was obtained in a yield of 90% (280 mg).

¹ NMR (400 MHz, Methanol-d₄) δ 7.48-7.39 (m, 2H), 7.15-7.04 (m, 2H), 4.02 (ddt, 2H), 3.69 (dd, 1H), 3.10- 2.70 (m, 4H), 1.47 (d, 10H), LC-MS: m/z 281.1 (MH+).

Preparation: tert-butyl 3-(2-chlorophenyl)piperazine-1-carboxylate

The synthesis of the title compound was effected analogously to the synthesis of tert-butyl 3-(3-fluorophenyl)piperazine-1-carboxylate wherein 2-(2-chlorophenyl)piperazine was used instead of 2-(3-fluorophenyl)piperazine. The title compound was obtained in a yield of 99% (752 mg).

1H NMR (400 MHz, DMSO-d6) δ ppm 7.68 (dd, J=7.59, 1.87 Hz, 1 H), 7.44 (dd, J=7.81, 1.43 Hz, 1 H), 7.40 -7.28 (m, 2 H), 4.03 (br d, J=12.10 Hz, 1 H), 3.97-3.81 (m, 2 H), 2.98 (br d, J=11.00 Hz, 1 H), 2.93-2.73 (m, 2 H), 2.73-2.63 (m, 2 H), 1.41 (s, 9 H); LC-MS: m/z 297.132 (MH+).

Preparation: tert-butyl 3-(3-chlorophenyl)piperazine-1-carboxylate

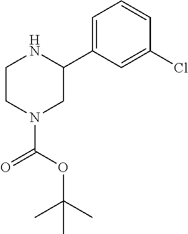 [SMILES: CC(C)(C)OC(=O)N1CCNC(c2cccc(Cl)c2)C1]

The synthesis of the title compound was effected analogously to the synthesis of tert-butyl 3-(3-fluorophenyl)piperazine-1-carboxylate wherein 2-(3-chlorophenyl)piperazine was used instead of 2-(3-fluorophenyl)piperazine. The title compound was obtained in a yield of 99% (752 mg).

1H NMR (400 MHz, DMSO-d6) δ ppm 7.48 (s, 1 H), 7.44-7.31 (m, 4 H), 3.81 (br d, J=12.32 Hz, 2 H), 3.60 (dd, J=10.34, 2.86 Hz, 1 H), 3.03-2.73 (m, 2 H), 2.73-2.59 (m, 2 H), 1.41 (s, 9 H); LC-MS: 297.16 (MH+).

Preparation: tert-butyl 4-[2-(azepan-1-yl)-4-(cyclopropanecarbonylamino)benzoyl]-2-thiophen-2-ylpiperazine-1-carboxylate

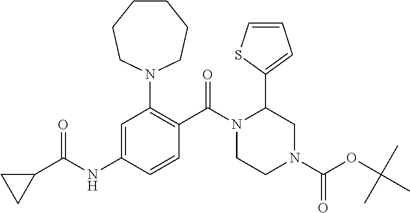 [SMILES: CC(C)(C)OC(=O)N1CCN(C(=O)c2ccc(NC(=O)C3CC3)cc2N2CCCCCC2)C(c2cccs2)C1]

### Example 124

The synthesis of the title compound was effected analogously to the synthesis of the compound of Example 35 (N-(3-(azepan-1-yl)-4-(piperidine-1-carbonyl)phenyl)cyclopropanecarboxamide) wherein tert-butyl 2-thiophen-2-ylpiperazine-1-carboxylate was used instead of piperidine. The title compound was obtained in a yield of 89% (145 mg).
LC-MS: m/z 553.4 (MH+)
   Preparation: tert-butyl 4-[2-(azepan-1-yl)-4-(cyclopropanecarbonylamino)benzoyl]-3-thiophen-3-ylpiperazine-1-carboxylate

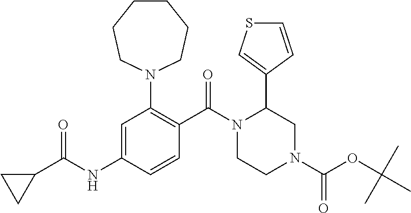 [SMILES: CC(C)(C)OC(=O)N1CCN(C(=O)c2ccc(NC(=O)C3CC3)cc2N2CCCCCC2)C(c2ccsc2)C1]

### Example 125

The synthesis of the title compound was effected analogously to the synthesis of the compound of Example 35 (N-(3-(azepan-1-yl)-4-(piperidine-1-carbonyl)phenyl)cyclopropanecarboxamide) wherein tert-butyl 3-thiophen-2-ylpiperazine-1-carboxylate was used instead of piperidine. The title compound was obtained in a yield of 80% (65 mg).
LC-MS: m/z 553.4 (MH+).
   Preparation: tert-butyl 4-[2-(azepan-1-yl)-4-(cyclopropanecarbonylamino)benzoyl]-3-(1-methylpyrazol-4-yl)piperazine-1-carboxylate

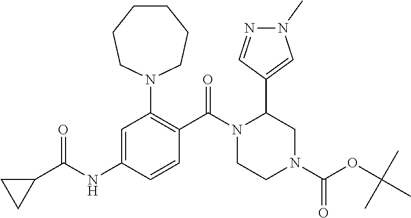 [SMILES: Cn1cc(C2CN(C(=O)OC(C)(C)C)CCN2C(=O)c2ccc(NC(=O)C3CC3)cc2N2CCCCCC2)cn1]

### Example 126

The synthesis of the title compound was effected analogously to the synthesis of the compound of Example 35 (N-(3-(azepan-1-yl)-4-(piperidine-1-carbonyl)phenyl)cyclopropanecarboxamide) wherein tert-butyl 3-(1-methylpyrazol-4-yl)piperazine-1-carboxylate was used instead of piperidine. The title compound was obtained in a yield of 58% (70 mg).
LC-MS: m/z 551.4 (MH+).
   Preparation: tert-butyl 4-[2-(azepan-1-yl)-4-(cyclopropanecarbonylamino)benzoyl]-3-pyridin-2-ylpiperazine-1-carboxylate

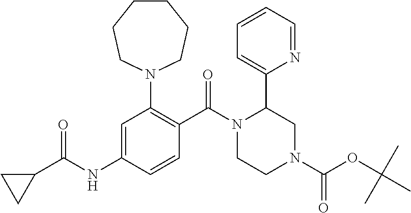 [SMILES: CC(C)(C)OC(=O)N1CCN(C(=O)c2ccc(NC(=O)C3CC3)cc2N2CCCCCC2)C(c2ccccn2)C1]

### Example 127

The synthesis of the title compound was effected analogously to the synthesis of the compound of Example 35 (N-(3-(azepan-1-yl)-4-(piperidine-1-carbonyl)phenyl)cyclopropanecarboxamide) wherein tert-butyl 3-pyridin-2-ylpiperazine-1-carboxylate was used instead of piperidine. The title compound was obtained in a yield of 50% (80 mg).
LC-MS: m/z 548.52
   Preparation: tert-butyl 4-[2-(azepan-1-yl)-4-(cyclopropanecarbonylamino)benzoyl]-3-(trifluoromethyl)piperazine-1-carboxylate

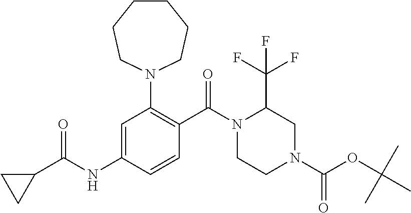 [SMILES: CC(C)(C)OC(=O)N1CCN(C(=O)c2ccc(NC(=O)C3CC3)cc2N2CCCCCC2)C(C(F)(F)F)C1]

### Example 128

The synthesis of the title compound was effected analogously to the synthesis of the compound of Example 35 (N-(3-(azepan-1-yl)-4-(piperidine-1-carbonyl)phenyl)cyclopropanecarboxamide) wherein tert-butyl 3-(trifluoromethyl)piperazine-1-carboxylate was used instead of piperidine. The title compound was obtained in a yield of 40% (45 mg).
LC-MS: m/z 539.4 (MH+)
   Preparation: tert-butyl 4-[2-(azepan-1-yl)-4-(cyclopropanecarbonylamino)benzoyl]-3-benzylpiperazine-1-carboxylate

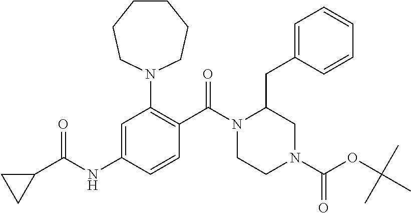 [SMILES: CC(C)(C)OC(=O)N1CCN(C(=O)c2ccc(NC(=O)C3CC3)cc2N2CCCCCC2)C(Cc2ccccc2)C1]

### Example 129

The synthesis of the title compound was effected analogously to the synthesis of the compound of Example 35 (N-(3-(azepan-1-yl)-4-(piperidine-1-carbonyl)phenyl)cyclopropanecarboxamide) wherein tert-butyl 3-benzylpiperazine-1-carboxylate was used instead of piperidine. The title compound was obtained in a yield of 88% (100 mg).
LC-MS: m/z 561.4 (MH+)
   Preparation: tert-butyl 4-[2-(azepan-1-yl)-4-(cyclopropanecarbonylamino)benzoyl]-3-(hydroxymethyl)piperazine-1-carboxylate

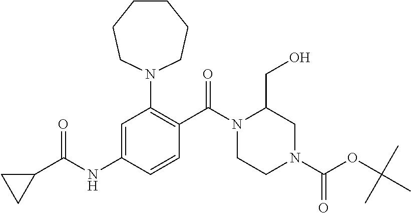 [SMILES: CC(C)(C)OC(=O)N1CCN(C(=O)c2ccc(NC(=O)C3CC3)cc2N2CCCCCC2)C(CO)C1]

### Example 130

The synthesis of the title compound was effected analogously to the synthesis of the compound of Example 35 (N-(3-(azepan-1-yl)-4-(piperidine-1-carbonyl)phenyl)cyclopropanecarboxamide) wherein 1-boc-3-hydroxymethyl-piperizine was used instead of piperidine. The title compound was obtained in a yield of 72% (80 mg).
LC-MS: m/z 501.3 (MH+)
   Preparation: tert-butyl 4-[2-(azepan-1-yl)-4-(cyclopropanecarbonylamino)benzoyl]-3-methylpiperazine-1-carboxylate

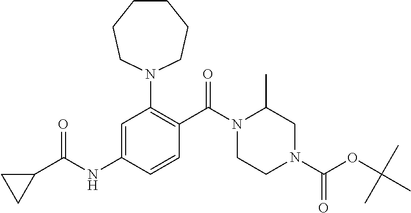 [SMILES: CC1CN(C(=O)OC(C)(C)C)CCN1C(=O)c1ccc(NC(=O)C2CC2)cc1N1CCCCCC1]

### Example 131

The synthesis of the title compound was effected analogously to the synthesis of the compound of Example 35 (N-(3-(azepan-1-yl)-4-(piperidine-1-carbonyl)phenyl)cyclopropanecarboxamide) wherein 3-methyl-1-piperazinecarboxylic acid tert-butyl ester was used instead of piperidine. The title compound was obtained in a yield of 98% (105 mg).

Preparation: tert-butyl 4-[2-(azepan-1-yl)-4-(cyclopropanecarbonylamino)benzoyl]-3-propan-2-ylpiperazine-1-carboxylate

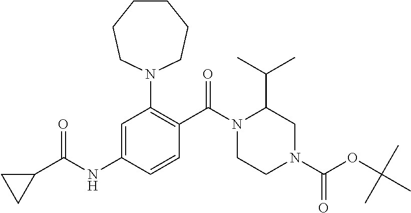 [SMILES: CC(C)C1CN(C(=O)OC(C)(C)C)CCN1C(=O)c1ccc(NC(=O)C2CC2)cc1N1CCCCCC1]

### Example 132

The synthesis of the title compound was effected analogously to the synthesis of the compound of Example 35 (N-(3-(azepan-1-yl )-4-(piperidine-1-carbonyl)phenyl)cyclopropanecarboxamide) wherein tert-butyl 3-propan-2-ylpiperazine-1-carboxylate was used instead of piperidine. The title compound was obtained in a yield of 99% (120 mg).
LC-MS: m/z 513.4 (MH+)
   Preparation: tert-butyl 4-[2-(azepan-1-yl)-4-(cyclopropanecarbonylamino)benzoyl]-3-phenylpiperazine-1-carboxylate

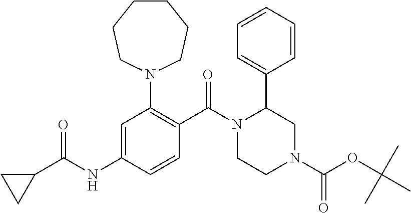 [SMILES: CC(C)(C)OC(=O)N1CCN(C(=O)c2ccc(NC(=O)C3CC3)cc2N2CCCCCC2)C(c2ccccc2)C1]

### Example 133

The synthesis of the title compound was effected analogously to the synthesis of the compound of Example 35 (N-(3-(azepan-1-yl)-4-(piperidine-1-carbonyl)phenyl)cyclopropanecarboxamide) wherein tert-butyl 3-phenyl-piperazine-1-carboxylic acid tert-butyl ester was used instead of piperidine. The title compound was obtained in a yield of 91% (145 mg).
LC-MS: m/z 547.5 (MH+) eparation: tert-butyl 4-[2-(azepan-1-yl)-4-(cyclopropanecarbonylamino)benzoyl]-3-pyridin-2-ylpiperazine-1-carboxylate

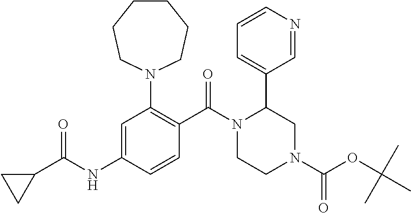 [SMILES: CC(C)(C)OC(=O)N1CCN(C(=O)c2ccc(NC(=O)C3CC3)cc2N2CCCCCC2)C(c2cccnc2)C1]

### Example 134

The synthesis of the title compound was effected analogously to the synthesis of the compound of Example 35 (N-(3-(azepan-1-yl)-4-(piperidine-1-carbonyl)phenyl)cyclopropanecarboxamide) wherein tert-butyl 3-pyridin-2-ylpiperazine-1-carboxylate was used instead of piperidine. The title compound was obtained in a yield of 91% (145 mg).
LC-MS: m/z 548.5 (MH+)
   Preparation: tert-butyl 4-[4-(cyclopropanecarbonylamino)-2-pyrrolidin-1-ylbenzoyl]-3-phenylpiperazine-1-carboxylate

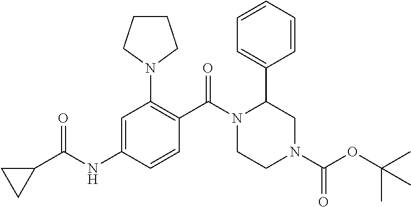 [SMILES: CC(C)(C)OC(=O)N1CCN(C(=O)c2ccc(NC(=O)C3CC3)cc2N2CCCC2)C(c2ccccc2)C1]

### Example 135

4-(cyclopropanecarbonylamino)-2-pyrrolidin-1-ylbenzoic acid (100.0 mg, 0.360 mmol), HATU: [dimethylamino(3-triazolo[4,5-b]pyridinyloxy)methylidene]-dimethylammonium hexafluorophosphate (166.33 mg, 0.440 mmol) and N,N-Diisopropylethylamine (0.25 mL, 1.46 mmol) were mixed in DMF (3.5 mL) and stirred for 5 min, then 3-phenylpiperazine, N1 Coc protected (95.64 mg, 0.360 mmol) was added. The reaction mixture was stirred at RT for 2h. stirred ON at 60° C. The day after the reaction was cooled down to RT, poured into a H₂O and extracted with AcOEt 3 times. The organic fractions were combined, washed with Brine, dried over Na₂SO₄ and concentrated under vacuum. The residue was purified by FC on NH column (eluting from 100% of cHex to AcOEt/cHex 3:7) affording the product of formula tert-butyl 4-[4-(cyclopropanecarbonylamino)-2-pyrrolidin-1-yl benzoyl]-3-phenylpiperazine-1-carboxylate as a racemic mixture.
Yield: 156 mg.

¹ NMR (400 MHz, DMSO-d₆) δ 10.19-10.04 (m, 1H), 7.49-6.69 (m, 8H), 5.76-4.31 (m, 2H), 3.74-3.48 (m, 2H), 3.31 (s, 2H), 3.17-2.86 (m, 4H), 2.05-1.60 (m, 5H), 1.48-1.19 (m, 10H), 0.78 (d, 4H), LC-MS: 519.2 (MH+)

Preparation: tert-butyl 4-[4-(cyclopropanecarbonylamino)-2-pyrrolidin-1-ylbenzoyl]-3-(4-fluorophenyl)piperazine-1-carboxylate

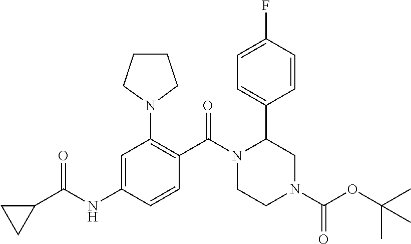 [SMILES: CC(C)(C)OC(=O)N1CCN(C(=O)c2ccc(NC(=O)C3CC3)cc2N2CCCC2)C(c2ccc(F)cc2)C1]

### Example 136

To a solution of 4-(cyclopropanecarbonylamino)-2-pyrrolidin-1-ylbenzoic acid (136.5 mg, 0.500 mmol) and HATU: [dimethylamino(3-triazolo[4,5-b]pyridinyloxy)methylidene]-dimethylammonium hexafluorophosphate (283.81 mg, 0.750 mmol) in DMF (3 mL), sequentially N,N-Diisopropylethylamine (0.26 mL, 1.49 mmol) and tert-butyl 3-(4-fluorophenyl)piperazine-1-carboxylate (150.0 mg, 0.500 mmol) were added. The reaction mixture was heated to 80° C. and left to react ON. After completion of the reaction the mixture was cooled down to RT and then it was poured into a s. s. of NaHCO₃ and AcOEt. Phases were separated and the organic layes was washed with Brine, dried over Na₂SO₄,filtered and concentrate to dryness under vacuum affording the product of formula tert-butyl 4-[4-(cyclopropanecarbonylamino)-2-pyrrolidin-1-ylbenzoyI]-3-(4-fluorophenyl)piperazine-1-carboxylate as a racemic mixture. The product was used in the next step without further purification.
Yield: 110 mg.

¹H NMR (400 MHz, Chloroform-d) δ 7.63-7.26 (m, 3H), 7.04 (ddtd, 5H), 6.82-5.15 (m, 2H), 4.87-4.35 (m, 1H), 4.20-3.66 (m, 2H), 3.59-2.68 (m, 6H), 1.88 (d, 4H), 1.57-1.43 (m, 9H), 1.29 (d, 1H), 1.15-1.02 (m, 2H), 0.94-0.75 (m, 2H), LC-MS: 537.18 (MH+)

Preparation: Tert-butyl 4-[4-(cyclopropanecarbonylamino)-2-pyrrolidin-1-ylbenzoyl]-3-(3-fluorophenyl)piperazine-1-carboxylate

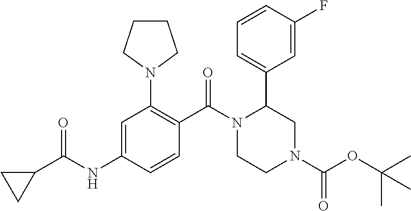 [SMILES: CC(C)(C)OC(=O)N1CCN(C(=O)c2ccc(NC(=O)C3CC3)cc2N2CCCC2)C(c2cccc(F)c2)C1]

### Example 137

The synthesis of the title compound was effected analogously to the synthesis of the compound of Example 136 (tert-butyl 4-[4-(cyclopropanecarbonylamino)-2-pyrrolidin-1-ylbenzoyI]-3-(4-fluorophenyl)piperazine-1-carboxylate) wherein tert-butyl 3-(3-fluorophenyl)piperazine-1-carboxylate was used instead of tert-butyl 3-(4-fluorophenyl)piperazine-1-carboxylate. The title compound was obtained in a yield of 52.2% (100 mg) as a racemic mixture.
LC-MS: m/z 537.24 (MH+).
   Preparation: tert-butyl 4[4-(cyclopropanecarbonylamino)-2-pyrrolidin-1-ylbenzoyl]-3-(2-fluorophenyl)piperazine-1-carboxylate

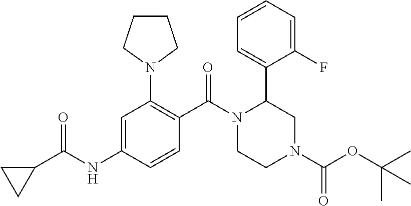 [SMILES: CC(C)(C)OC(=O)N1CCN(C(=O)c2ccc(NC(=O)C3CC3)cc2N2CCCC2)C(c2ccccc2F)C1]

### Example 138

The synthesis of the title compound was effected analogously to the synthesis of the compound of Example 136 (tert-butyl 4-[4-(cyclopropanecarbonylamino)-2-pyrrolidin-1-ylbenzoyI]-3-(4-fluorophenyl)piperazine-1-carboxylate) wherein tert-butyl 3-(2-fluorophenyl)piperazine-1-carboxylate was used instead of tert-butyl 3-(4-fluorophenyl)piperazine-1-carboxylate. The title compound was obtained in a yield of 79% (200 mg) as a racemic mixture.
LC-MS: m/z 537.26 (MH+).
   Preparation: tert-butyl 4-[2-(6-azaspiro[3.4]octan-6-y0-4-(cyclopropanecarbonylamino)benzoyl]-3-thiophen-2-ylpiperazine-1-carboxylate

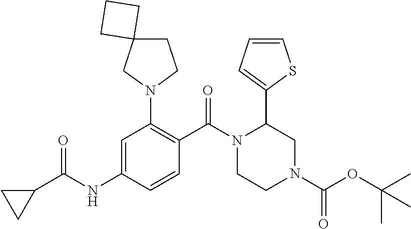 [SMILES: CC(C)(C)OC(=O)N1CCN(C(=O)c2ccc(NC(=O)C3CC3)cc2N2CCC3(CCC3)C2)C(c2cccs2)C1]

### Example 139

The synthesis of the title compound was effected analogously to the synthesis of the compound of Example 136 (tert-butyl 4-[4-(cyclopropanecarbonylamino)-2-pyrrolidin-1-ylbenzoyI]-3-(4-fluorophenyppiperazine-1-carboxylate) wherein tert-butyl 3-thiophen-2-ylpiperazine-1-carboxylate and 2-(6-azaspiro[3.4]octan-6-yl)-4-(cyclopropanecarbonylamino)benzoic acid were used instead of tert-butyl 3-(4-fluorophenyl)piperazine-1-carboxylate and resp4-(cyclopropanecarbonylamino)-2-pyrrolidin-1-ylbenzoic acid respectively. The title compound was obtained in a yield of 22% (28 mg) as a racemic mixture.
LC-MS: m/z 565.2 (MH+).
   Preparation: tert-butyl 4-[4-(cyclopropanecarbonylamino)-2-pyrrolidin-1-ylbenzoyl]-3-thiophen-2-ylpiperazine-1-carboxylate

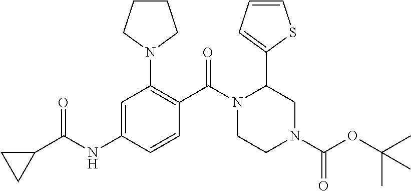 [SMILES: CC(C)(C)OC(=O)N1CCN(C(=O)c2ccc(NC(=O)C3CC3)cc2N2CCCC2)C(c2cccs2)C1]

### Example 140

The synthesis of the title compound was effected analogously to the synthesis of the compound of Example 136 (tert-butyl 4-[4-(cyclopropanecarbonylamino)-2-pyrrolidin-1-ylbenzoyI]-3-(4-fluorophenyl)piperazine-1-carboxylate) wherein tert-butyl 3-thiophen-2-ylpiperazine-1-carboxylate was used instead of tert-butyl 3-(4-fluorophenyl)piperazine-1-carboxylate. The title compound was obtained in a yield of 99% (28 mg) as a racemic mixture.
LC-MS: m/z 525.19 (MH+)
   Preparation: tert-butyl 4[4-(cyclopropanecarbonylamino)-2-pyrrolidin-1-ylbenzoyl]-3-(1-methylpyrazol-4-yppiperazine-1-carboxylate

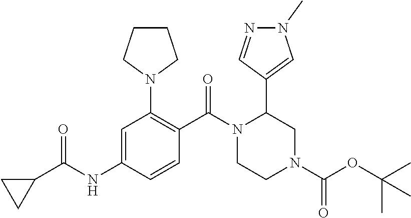 [SMILES: Cn1cc(C2CN(C(=O)OC(C)(C)C)CCN2C(=O)c2ccc(NC(=O)C3CC3)cc2N2CCCC2)cn1]

### Example 141

The synthesis of the title compound was effected analogously to the synthesis of the compound of Example 136 (tert-butyl 4-[4-(cyclopropanecarbonylamino)-2-pyrrolidin-1-ylbenzoyI]-3-(4-fluorophenyl)piperazine-1-carboxylate) wherein tert-butyl 3-(1-methylpyrazol-4-yl)piperazine-1-carboxylate was used instead of tert-butyl 3-(4-fluorophenyl)piperazine-1-carboxylate. The title compound was obtained in a yield of 99% (28 mg) as a racemic mixture. LC-MS: m/z 523.24 (MH+).

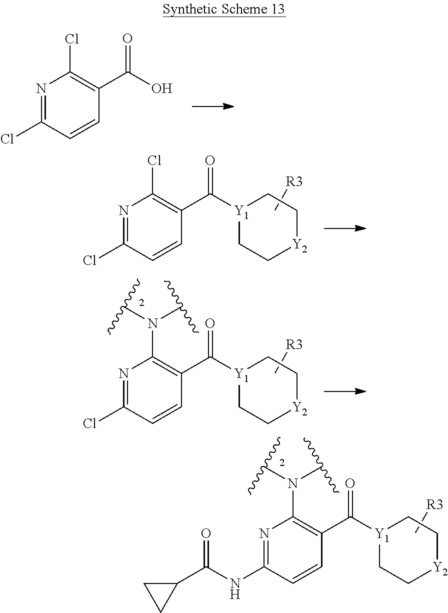

Preparation: (2,6-dichloropyridin-3-yl)-(1,1-dioxo-1,4-thiazinan-4-yl)methanone

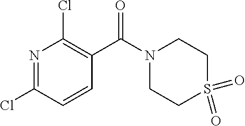 [SMILES: O=C(c1ccc(Cl)nc1Cl)N1CCS(=O)(=O)CC1]

In a round bottom flask 2,6-dichloro-3-pyridinecarboxylic acid (1.0 g, 5.21 mmol) and thionyl dichloride (3.8 mL, 52.08 mmol) were mixed together. The suspension was heated to reflux and stirred for 30 min. After this time the reaction mixture was cooled down to r.t. and concentrated under reduced pressure. The resulting material was stripped three times with cHex. The residue was dissolved in DCM (9.091 mL), cooled down to 0° C. and stirred for 10 min. After this time a solution of N,N-Diisopropylethylamine (2.48 mL, 14.26 mmol) and thiomorpholine 1,1-dioxide hydrochloride (642.34 mg, 4.75 mmol) in DCM (2 mL) was added over 10 minutes. The resulting solution was warm to RT and stirred for 30 min. The reaction mixture was diluted with DCM, washed with water, dried over Na₂SO₄, filtered and concentrated under reduced pressure. The residue was purified by FC on silica gel (eluting from 100% of cHex to cHex/AcOEt 4:6) affording the product of formula (2,6-dichloropyridin-3-yl)-(1,1-dioxo-1,4-thiazinan-4-yl)methanone
Yield: 1 g

1H NMR (400 MHz, CHLOROFORM-d) δ 7.67 (d, 1 H), 7.43 (d, 1 H), 4.48-4.65 (m, 1 H), 4.19-4.04 (m, 1 H), 3.92-3.70 (m, 2 H), 3.31-3.13 (m, 3 H), 3.05-2.90 (m, 1 H); LC-MS: 309.24 (MH+)

Preparation: (2,6-dichloropyridin-3-yl)-(4-methyl-2-phenylpiperazin-1-yl)methanone

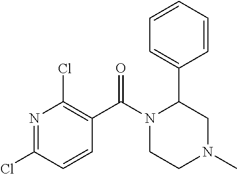 [SMILES: CN1CCN(C(=O)c2ccc(Cl)nc2Cl)C(c2ccccc2)C1]

A suspension of 2,6-dichloro-3-pyridinecarboxylic acid (3.0 g, 15.63 mmol) in thionyl dichloride (11.33 mL, 156.25 mmol) was heated to reflux and stirred for 30 min. After this time the reaction mixture was cooled down to room temperature and concentrated under reduced pressure, the residue was taken up with cHex and concentrated under vacuum. This action was repeated 3 times. The residue was then suspended in DCM (25 mL) and N,N-Diisopropylethylamine (2.37 mL, 13.62 mmol) was added. The reaction was cooled to 0° C. and 1-methyl-3-phenylpiperazine (2.0 g, 11.35 mmol) was added. The reaction was warmed to RT and stirred for 3 h. After this time the reaction was diluted with DCM and washed with a s.s. of NaHCO₃. The organic phase was dried through phase separator and concentrated under vacuum. The residue was purified by FC on silica gel (eluting from 100% of cHex to cHex/AcOEt 1:1) affording the product of formula (2,6-dichloropyridin-3-yl)-(4-methyl-2-phenylpiperazin-1-yl)methanone as a racemic mixture.
Yield: 3 g

H NMR (400 MHz, DMSO-d6)δ 8.05 (dd, 1H), 7.88-7.45 (m, 3H), 7.43-7.13 (m, 3H), 4.75-4.30 (m, 1H), 3.54-3.36 (m, 1H), 3.24-2.61 (m, 3H), 2.44-2.14 (m, 4H), 2.09-1.90 (m, 1H); LC-MS: m/z 350.1

Preparation: [6-chloro-2-(3-propan-2-ylpyrrolidin-1-yl)pyridin-3-yl]-(1,1-dioxo-1,4-thiazinan-4-yl)methanone

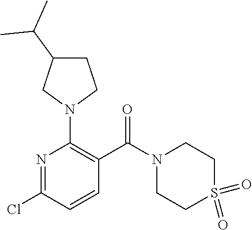 [SMILES: CC(C)C1CCN(c2nc(Cl)ccc2C(=O)N2CCS(=O)(=O)CC2)C1]

To a solution of (2,6-dichloropyridin-3-yl)-(1,1-dioxo-1,4-thiazinan-4-yl)methanone (155.0 mg, 0.500 mmol) in DMF (1 mL) 3-(propan-2-yl)pyrrolidine hydrochloride and potassium carbonate (69.29 mg, 0.500 mmol) were added. The resulting mixture was stirred at 80° C. ON. The day after H₂O was added and the reaction was extracted with AcOEt 3 times. The combined organic fractions were Brine, dried over Na₂SO₄, filtered and concentrated under reduced pressure. The residue was Purified FC on silica gel (eluenting from cHex/AcOEt 9:1 to cHex/AcOEt 6:4) affording the product of formula [6-chloro-2-(3-propan-2-ylpyrrolidin-1-yl)pyridin-3-yl]-(1,1-dioxo-1,4-thiazinan-4-yl)methanone as a racemic mixture.
Yield: 79 mg
LC-MS: m/z 386.1 (MH+)
   Preparation: [2-(6-azaspiro[3.4]octan-6-yl)-6-chloropyridin-3-yl]-(1,1-dioxo-1,4-thiazinan-4-yl)methanone

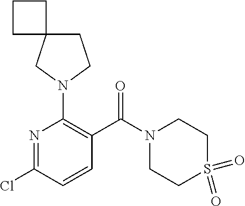 [SMILES: O=C(c1ccc(Cl)nc1N1CCC2(CCC2)C1)N1CCS(=O)(=O)CC1]

The synthesis of the title compound was effected analogously to the synthesis of [6-chloro-2-(3-propan-2-ylpyrrolidin-1-yl)pyridin-3-yl]-(1,1-dioxo-1,4-thiazinan-4-yl)methanone wherein 6-aza-spiro[3.4]octane was used instead of 3-(propan-2-yl)pyrrolidine hydrochloride. The title compound was obtained in a yield of 58% (112 mg).

1H NMR (400 MHz, CHLOROFORM-d),

¹ NMR (400 MHz, Chloroform-d) δ 7.36 (d, 1H), 6.65 (d, 1H), 4.04-3.70 (m, 4H), 3.47 (s, 2H), 3.32 (s, 2H), 3.16 (s, 2H), 2.95 (s, 2H), 2.04-1.90 (m, 8H); LC-MS: 384.2 (MH+)

Preparation: (6-chloro-2-pyrrolidin-1-ylpyridin-3-yl)-(4-methyl-2-phenylpiperazin-1-yl)methanone

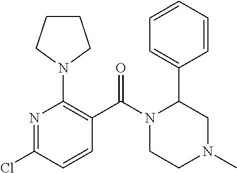 [SMILES: CN1CCN(C(=O)c2ccc(Cl)nc2N2CCCC2)C(c2ccccc2)C1]

The synthesis of the title compound was effected analogously to the synthesis of [6-chloro-2-(3-propan-2-ylpyrrolidin-1-yl)pyridin-3-yl]-(1,1-dioxo-1,4-thiazinan-4-yl)methanone wherein (2,6-dichloropyridin-3-yl)-(4-methyl-2-phenylpiperazin-1-yl)methanone and pyrrolidine were used instead of (2,6-dichloropyridin-3-yl)-(1,1-dioxo-1,4-thiazinan-4-yl)methanone and 3-(propan-2-yl)pyrrolidine hydrochloride respectively. The title compound was obtained in a yield of 60% (190 mg) as a racemic mixture.

1H NMR (400 MHz, CHLOROFORM-d) δ 7.97-7.03 (m, 6 H), 6.64-6.32 (m, 1 H), 6.08 -4.74 (m, 1 H), 4.72-2.63 (m, 8 H), 2.54-2.36 (m, 1 H), 2.35-2.26 (m, 3 H), 2.12-2.02 (m, 1 H), 2.02-1.62 (m, 4 H);
LC-MS: m/z 385.2 (MH+).
   Preparation: [2-(3-azabicyclo[3.1.0]hexan-3-yl)-6-chloropyridin-3-yl]-(4-methyl-2-phenylpiperazin-1-yl)methanone

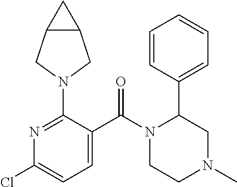 [SMILES: CN1CCN(C(=O)c2ccc(Cl)nc2N2CC3CC3C2)C(c2ccccc2)C1]

The synthesis of the title compound was effected analogously to the synthesis of [6-chloro-2-(3-propan-2-ylpyrrolidin-1-yl)pyridin-3-yl]-(1,1-dioxo-1,4-thiazinan-4-yl)methanone wherein (2,6-dichloropyridin-3-yl)-(4-methyl-2-phenylpiperazin-1-yl)methanone and 3-Azabicyclo[3.1.0]hexane hydrochloride were used instead of (2,6-dichloropyridin-3-yl)-(1,1-dioxo-1,4-thiazinan-4-yl)methanone and 3-(propan-2-yl)pyrrolidine hydrochloride respectively. The title compound was obtained in a yield of 53% (182 mg) as a racemic mixture.
LC-MS: m/z 397.17
   Preparation: [6-chloro-2-(4,4-difluoropiperidin-1-yl)pyridin-3-yl]-(4-methyl-2-phenylpiperazin-1-yl)methanone

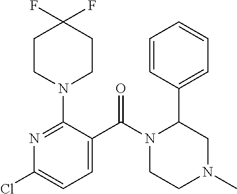 [SMILES: CN1CCN(C(=O)c2ccc(Cl)nc2N2CCC(F)(F)CC2)C(c2ccccc2)C1]

The synthesis of the title compound was effected analogously to the synthesis of [6-chloro-2-(3-propan-2-ylpyrrolidin-1-yl)pyridin-3-yl]-(1,1-dioxo-1,4-thiazinan-4-yl)methanone wherein (2,6-dichloropyridin-3-yl)-(4-methyl-2-phenylpiperazin-1-yl)methanone and 4,4-difluoropiperidine were used instead of (2,6-dichloropyridin-3-yl)-(1,1-dioxo-1,4-thiazinan-4-yl)methanone and 3-(propan-2-yl)pyrrolidine hydrochloride respectively. The title compound was obtained in a yield of 44% (167 mg) as a racemic mixture.

¹ NMR (400 MHz, DMSO-d₆) δ 7.84-7.58 (m, 1H), 7.53-7.42 (m, 1H), 7.39-7.17 (m, 4H), 7.06-6.80 (m, 1H), 5.83-5.61 (m, 1H), 3.54 (d, 2H), 3.27-2.60 (m, 5H), 2.44 —1.88 (m, 8H), 1.79-1.51 (m, 2H), LC-MS: m/z 435.16 (MH+).

Preparation: [6-chloro-2-(3,3-difluoropiperidin-1-yl)pyridin-3-yl]-(4-methyl-2-phenylpiperazin-1-yl)methanone

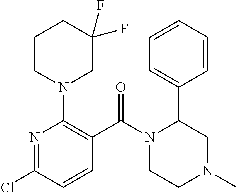 [SMILES: CN1CCN(C(=O)c2ccc(Cl)nc2N2CCCC(F)(F)C2)C(c2ccccc2)C1]

The synthesis of the title compound was effected analogously to the synthesis of [6-chloro-2-(3-propan-2-ylpyrrolidin-1-yl )pyridin-3-yl]-(1,1-dioxo-1,4-thiazinan-4-yl)methanone wherein (2,6-dichloropyridin-3-yl)-(4-methyl-2-phenylpiperazin-1-yl )methanone and 3,3-difluoropiperidine were used instead of (2,6-dichloropyridin-3-yl)-(1,1-dioxo-1,4-thiazinan-4-yl)methanone and 3-(propan-2-yl)pyrrolidine hydrochloride respectively. The title compound was obtained in a yield of 35% (75 mg) as a racemic mixture.

¹ NMR (400 MHz, DMSO-d₆) δ 7.80-7.61 (m, 1H), 7.48 (dd, 1H), 7.43 —7.16 (m, 4H), 6.86 (dd, 1H), 5.84-4.34 (m, 1H), 3.92-3.38 (m, 3H), 3.22-2.58 (m, 5H), 2.45-1.69 (m, 8H), 1.53-1.08 (m, 1H), LC-MS: m/z 435.16 (MH+).

Preparation: [6-chloro-2-(3,3-difluoropyrrolidin-1-yl)pyridin-3-yl]-(4-methyl-2-phenylpiperazin-1-yl)methanone

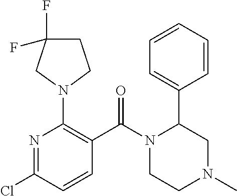 [SMILES: CN1CCN(C(=O)c2ccc(Cl)nc2N2CCC(F)(F)C2)C(c2ccccc2)C1]

The synthesis of the title compound was effected analogously to the synthesis of [6-chloro-2-(3-propan-2-ylpyrrolidin-1-yl)pyridin-3-yl]-(1,1-dioxo-1,4-thiazinan-4-yl)methanone wherein (2,6-dichloropyridin-3-yl)-(4-methyl-2-phenylpiperazin-1-yl)methanone and 3,3-difluoropyrrolidine hydrochloride were used instead of (2,6-dichloropyridin-3-yl)-(1,1-dioxo-1,4-thiazinan-4-yl)methanone and 3-(propan-2-yl)pyrrolidine hydrochloride respectively. The title compound was obtained in a yield of 30% (96 mg) as a racemic mixture..

1H NMR (500 MHz, DMSO-d6) δ 7.62-7.48 (m, 1 H), 7.96-7.11 (m, 5 H), 6.65 (br d, 1 H), 4.69-5.90 (m, 1 H), 4.58-3.01 (m, 7 H), 2.84-2.57 (m, 1 H), 2.56-2.47 (m, 1 H), 2.46-2.27 (m, 1 H), 2.28-2.12 (m, 4 H), 2.11-1.88 (m, 1 H); LC-MS: 421.1 (MH+).

Preparation: (6-chloro-2-piperidin-1-ylpyridin-3-yl)-(4-methyl-2-phenylpiperazin-1-yl)methanone

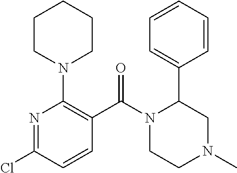 [SMILES: CN1CCN(C(=O)c2ccc(Cl)nc2N2CCCCC2)C(c2ccccc2)C1]

The synthesis of the title compound was effected analogously to the synthesis of [6-chloro-2-(3-propan-2-ylpyrrolidin-1-yl)pyridin-3-yl]-(1,1-dioxo-1,4-thiazinan-4-yl)methanone wherein (2,6-dichloropyridin-3-yl)-(4-methyl-2-phenylpiperazin-1-yl)methanone and piperidine were used instead of (2,6-dichloropyridin-3-yl)-(1,1-dioxo-1,4-thiazinan-4-yl)methanone and 3-(propan-2-yl)pyrrolidine hydrochloride respectively. The title compound was obtained in a yield of 50% (170 mg) as a racemic mixture.
LC-MS: m/z 399.1 (MH+)

1H NMR (500 MHz, DMSO-d6) δ 7.57-7.39 (m, 1 H), 7.88-7.04 (m, 5 H), 6.91-6.69 (m, 1 H), 5.75 -4.48 (m, 1 H), 3.51-3.34 (m, 1 H), 4.46-2.88 (m, 2 H), 3.51-2.88 (m, 4 H), 2.85-2.64 (m, 1 H), 2.48-2.26 (m, 1 H), 2.25-2.10 (m, 3 H), 2.06-1.88 (m, 1 H), 1.78-1.13 (m, 6 H).

Preparation: N-[5-(1,1-dioxo-1,4-thiazinane-4-carbonyl)-6-(3-propan-2-ylpyrrolidin-1-yl)pyridin-2-yl]cyclopropanecarboxamide

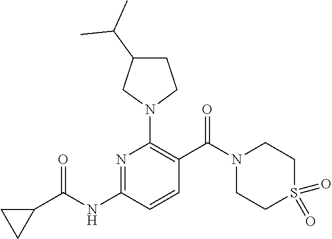 [SMILES: CC(C)C1CCN(c2nc(NC(=O)C3CC3)ccc2C(=O)N2CCS(=O)(=O)CC2)C1]

### Example 142

A solution of [6-chloro-2-(3-propan-2-ylpyrrolidin-1-yl )pyridin-3-yl]-(1,1-dioxo-1,4-thiazinan-4-yl)methanone (66.35 mg, 0.170 mmol), cyclopropanecarboxamide (16.09 mg, 0.190 mmol), dicesium;carbonate (112.73 mg, 0.340 mmol) and XPHOS PD G3 (7.28 mg, 0.010 mmol) in dry 1,4-Dioxane (1.715 mL) was degassed by Schlenk line technique. The resulting mixture was heated to 100° C. and stirred for 3h. After this time the reaction was cooled down to RT and the reaction mixture was diluted with AcOEt and filtered through a pad of celite. The filtrate was concentrated under reduced pressure and Purified by FC on silica gel (eluting from cHex/AcOEt 8:2 to cHex/AcOEt 4:6) affording the product of formula N-[5-(1,1-dioxo-1,4-thiazinane-4-carbonyl)-6-(3-propan-2-ylpyrrolidin-1-yl)pyridin-2-yl]cyclopropanecarboxamide as a racemic mixture.

1H NMR (500 MHz, DMSO-d6) δ ppm 10.38-10.16 (m, 1 H), 7.60-7.47 (m, 1 H), 7.40-7.24 (m, 1 H), 4.60-3.44 (m, 4 H), 3.77-2.90 (m, 4 H), 3.42-2.87 (m, 4 H), 2.17-1.96 (m, 2 H), 1.93-1.75 (m, 1 H), 1.57-1.39 (m, 2 H), 0.93 (d, 3 H), 0.91 (d, 3 H), 0.83-0.64 (m, 4 H); LC-MS: m/z 435.86 (MH+).

Preparation: N-[6-(6-azaspiro[3.4]octan-6-yl)-5-(1,1-dioxo-1,4-thiazinane-4-carbonyl)pyridin-2-yl]cyclopropanecarboxamide

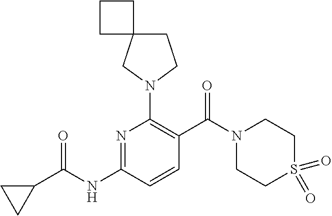 [SMILES: O=C(Nc1ccc(C(=O)N2CCS(=O)(=O)CC2)c(N2CCC3(CCC3)C2)n1)C1CC1]

### Example 143

The synthesis of the title compound was effected analogously to the synthesis of the compound of Example 142 (N-[5-(1,1-dioxo-1,4-thiazinane-4-carbonyl)-6-(3-propan-2-ylpyrrolidin-1-yl)pyridin-2-yl]cyclopropanecarboxamide) wherein [2-(6-azaspiro[3.4]octan-6-yl)-6-chloropyridin-3-yl]-(1,1-dioxo-1,4-thiazinan-4-yl)methanone was used instead of [6-chloro-2-(3-propan-2-ylpyrrolidin-1-yl)pyridin-3-yl]-(1,1-dioxo-1,4-thiazinan-4-yl)methanone. The title compound was obtained in a yield of 16.3% (20 mg).

1H NMR (500 MHz, DMSO-d6) δ ppm 10.31 (s, 1 H), 7.53 (d, 1 H), 7.30 (d, 1 H), 4.53-3.75 (m, 2 H), 3.61 (br t, 2 H), 3.47-3.15 (m, 4 H), 3.43-2.94 (m, 4 H), 2.09-2.03 (m, 1 H), 2.02-1.77 (m, 8 H), 0.84-0.70 (m, 4 H); LC-MS: 433.46 (MH+).

Preparation: N-[6-(3,3-difluoropiperidin-1-yl)-5-(4-methyl-2-phenylpiperazine-1-carbonyl)pyridin-2-yl]cyclopropanecarboxamide

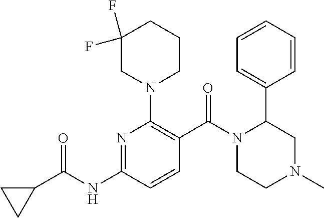 [SMILES: CN1CCN(C(=O)c2ccc(NC(=O)C3CC3)nc2N2CCCC(F)(F)C2)C(c2ccccc2)C1]

### Example 144

The synthesis of the title compound was effected analogously to the synthesis of the compound of Example 142 (N-[5-(1,1-dioxo-1,4-thiazinane-4-carbonyl)-6-(3-propan-2-ylpyrrolidin-1-yppyridin-2-yl]cyclopropanecarboxamide) wherein [6-chloro-2-(3,3-difluoropiperidin-1-yl)pyridin-3-yl]-(4-methyl-2-phenylpiperazin-1-yl)methanone was used instead of [6-chloro-2-(3-propan-2-ylpyrrolidin-1-yl)pyridin-3-yl]-(1,1-dioxo-1,4-thiazinan-4-yl)methanone. The title compound was obtained in a yield of 47% (69 mg) as a racemic mixture.

1H NMR (600 MHz, DMSO-d6) δ ppm 10.60-10.39 (m, 1 H), 7.62-7.39 (m, 2 H), 7.76-7.13 (m, 2 H), 7.48-7.11 (m, 3 H), 5.95-4.45 (m, 1 H), 4.43-2.86 (m, 7 H), 2.85-2.58 (m, 1 H), 2.48-2.26 (m, 1 H), 2.25-1.04 (m, 9 H), 0.91-0.63 (m, 4 H); LC-MS: m/z 484.19 (MH+).

Preparation: N-[6-(3-azabicyclo[3.1.0]hexan-3-yl)-5-(4-methyl-2-phenylpiperazine-1-carbonyppyridin-2-yl]cyclopropanecarboxamide

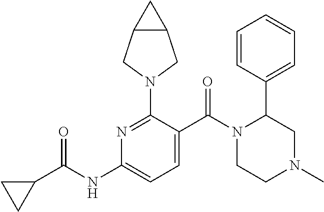 [SMILES: CN1CCN(C(=O)c2ccc(NC(=O)C3CC3)nc2N2CC3CC3C2)C(c2ccccc2)C1]

### Example 145

The synthesis of the title compound was effected analogously to the synthesis of the compound of Example 142 (N-[5-(1,1-dioxo-1,4-thiazinane-4-carbonyl)-6-(3-propan-2-ylpyrrolidin-1-yl)pyridin-2-yl]cyclopropanecarboxamide) wherein [2-(3-azabicyclo[3.1.0]hexan-3-yl)-6-chloropyridin-3-yl]-(4-methyl-2-phenylpiperazin-1-yl)methanone was used instead of [6-chloro-2-(3-propan-2-ylpyrrolidin-1-yl)pyridin-3-yl]-(1,1-dioxo-1,4-thiazinan-4-yl)methanone. The title compound was obtained in a yield of 61% (125 mg) as a racemic mixture.

1H NMR (600 MHz, DMSO-d6) δ ppm 10.56-9.96 (m, 1 H), 7.86-6.90 (m, 7 H), 5.92-4.63 (m, 1 H), 4.56-2.93 (m, 7 H), 2.92-2.62 (m, 1 H), 2.45-2.25 (m, 1 H), 2.25-2.11 (m, 3 H), 2.09-1.82 (m, 2 H), 1.71-1.29 (m, 2 H), 0.84-0.72 (m, 4 H), 0.70-0.43 (m, 1 H), 0.26 -0.23(m, 1 H); LC-MS: 446.2 (MH+).

Preparation N-[6-(4,4-difluoropiperidin-1-yl)-5-(4-methyl-2-phenylpiperazine-1-carbonyl)pyridin-2-yl]cyclopropanecarboxamide

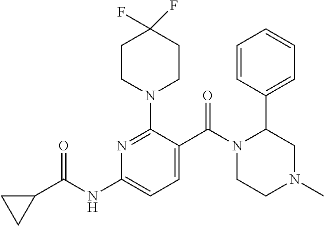 [SMILES: CN1CCN(C(=O)c2ccc(NC(=O)C3CC3)nc2N2CCC(F)(F)CC2)C(c2ccccc2)C1]

### Example 146

The synthesis of the title compound was effected analogously to the synthesis of the compound of Example 142 (N-[5-(1,1-dioxo-1,4-thiazinane-4-carbonyl)-6-(3-propan-2-ylpyrrolidin-1-yl)pyridin-2-yl]cyclopropanecarboxamide) wherein [[6-chloro-2-(4,4-difluoropiperidin-1-yl)pyridin-3-yl]-(4-methyl-2-phenylpiperazin-1-yl )methanone was used instead of [6-chloro-2-(3-propan-2-ylpyrrolidin-1-yl )pyridin yl]-(1,1-dioxo-1,4-thiazinan-4-yl)methanone. The title compound was obtained in a yield of 58% (106 mg) as a racemic mixture.

1H NMR (600 MHz, DMSO-d6) δ 10.51 (s, 1 H), 7.65-7.40 (m, 2 H), 7.80-7.13 (m, 2 H), 7.48-7.11- (m, 3 H), 5.88-4.50 (m, 1 H), 4.47-2.82 (m, 7 H), 2.84-2.63 (m, 1 H), 2.37-2.25 (m, 1 H), 2.25-1.56- (m, 9 H), 0.95-0.60 (m, 4 H); LC-MS: m/z 484.2 (MH+)

Preparation: N-[5-(4-methyl-2-phenylpiperazine-1-carbonyl)-6-piperidin-1-ylpyridin-2-yl]cyclopropanecarboxamide

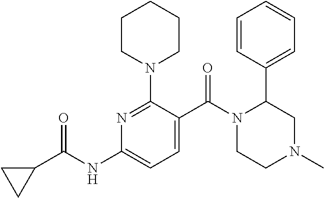 [SMILES: CN1CCN(C(=O)c2ccc(NC(=O)C3CC3)nc2N2CCCCC2)C(c2ccccc2)C1]

### Example 147

The synthesis of the title compound was effected analogously to the synthesis of the compound of Example 142 (N-[5-(1,1-dioxo-1,4-thiazinane-4-carbonyl)-6-(3-propan-2-ylpyrrolidin-1-yl)pyridin-2-yl]cyclopropanecarboxamide) wherein (6-chloro-2-piperidin-1-ylpyridin-3-yl)-(4-methyl-2-phenylpiperazin-1-yl)methanone was used instead of [6-chloro-2-(3-propan-2-ylpyrrolidin-1-yl)pyridin-3-yl]-(1,1-dioxo-1,4-thiazinan-4-yl)methanone. The title compound was obtained in a yield of 50% (95 mg) as a racemic mixture.

1H NMR (500 MHz, DMSO-d6) δ ppm 10.56-10.26 (m, 1 H), 7.93-6.96 (m, 7 H), 5.75-4.48 (m, 1 H), 4.43-2.84 (m, 7 H), 2.83-2.60 (m, 1 H), 2.57-2.26 (m, 1 H), 2.26-2.13 (m, 3 H), 2.13-1.86 (m, 2 H), 1.80-1.07 (m, 6 H), 0.78 (br d, 4 H); LC-MS: m/z 484.2 (MH+)

Preparation: N-[6-(3,3-difluoropyrrolidin-1-yl)-5-(4-methyl-2-phenylpiperazine-1-carbonyl)pyridin-2-yl]cyclopropanecarboxamide

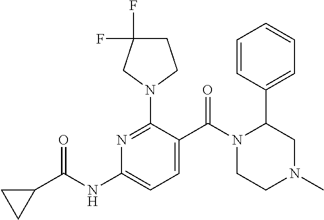 [SMILES: CN1CCN(C(=O)c2ccc(NC(=O)C3CC3)nc2N2CCC(F)(F)C2)C(c2ccccc2)C1]

### Example 148a, 148b, 148c

The synthesis of the title compound was effected analogously to the synthesis of the compound of Example 142 (N-[5-(1,1-dioxo-1,4-thiazinane-4-carbonyl)-6-(3-propan-2-ylpyrrolidin-1-yl)pyridin-2-yl]cyclopropanecarboxamide) wherein [6-chloro-2-(3,3-difluoropyrrolidin-1-yl)pyridin-3-yl]-(4-methyl-2-phenylpiperazin-1-yl)methanone was used instead of [6-chloro-2-(3-propan-2-ylpyrrolidin-1-yl)pyridin-3-yl]-(1,1-dioxo-1,4-thiazinan-4-yl)methanone. The title compound was obtained in a yield of 53% (65 mg) as a racemic mixture.

1H NMR (500 MHz, DMSO-d6) δ ppm 10.52-10.34 (m, 1 H), 7.77-7.18 (m, 7 H), 5.90-4.67 (m, 1 H), 4.60-2.86 (m, 34 H), 2.80-2.47 (m, 1 H), 2.69-2.45 (m, 1 H), 2.42-2.22 (m, 1 H), 2.29-2.17 (m, 3 H), 2.33-2.13 (m, 1 H), 2.09-1.98 (m, 1 H), 2.12-1.88 (m, 1 H), 0.88-0.72 (m, 4 H); LC-MS: m/z 470.1 (MH+).

The racemic mixture (Example 148c) was then separated into single enantiomers by semi preparative chiral HPLC

| | Column | Chiralpak AS-H (25 × 2.0 cm), 5 μMobile | |
|---|---|---|---|
| | phase | n-Hexane/Ethanol 85/15% v/v | |
| | Flow rate (ml/min) | 17 | ml/min |
| | DAD detection | 750 | nm |
| Preparative chiral | Loop | 500 | μL |
| chromatography | Total amount | 63 | mg |
| protocol: | Injection | 11.6 | mg (each injection) |

| | |
|---|---|
| 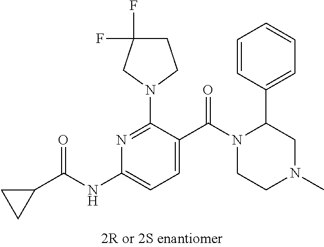 | Enantiomer 1 Rt 7.7 min 100% ee LC-MS: m/z 434.2 (MH+) (2R or 2S) N-[6-(3,3-difluoropyrrolidin-1-yl)-5-(4-methyl-2-phenylpiperazine- 1-carbonyl)pyridin-2-yl]cyclopropanecarboxamide Example 148a |
| 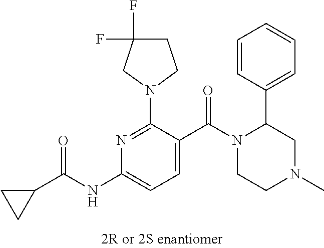 | Enantiomer 2 Rt 11.9 min 100% ee LC-MS: m/z 434.2 (MH+) (2R or 2S) N-[6-(3,3-difluoropyrrolidin-1-yl)-5-(4-methyl-2-phenylpiperazine- 1-carbonyl)pyridin-2-yl]cyclopropanecarboxamide Example 148b |

Preparation: N-[45-(4-methyl-2-phenylpiperazine-1-carbonyl)-6-pyrrolidin-1-ylpyridin-2-yl]cyclopropanecarboxamide

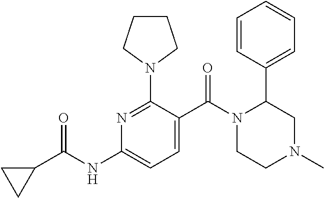 [SMILES: CN1CCN(C(=O)c2ccc(NC(=O)C3CC3)nc2N2CCCC2)C(c2ccccc2)C1]

### Example 149a, 149b, 149c

The synthesis of the title compound was effected analogously to the synthesis of the compound of Example 142 (N-[5-(1,1-dioxo-1,4-thiazinane-4-carbonyl)-6-(3-propan-2-ylpyrrolidin-1-yppyridin-2-yl]cyclopropanecarboxamide) wherein [(6-chloro-2-pyrrolidin-1-ylpyridin-3-yl)-(4-methyl-2-phenylpiperazin-1-yl]methanone was used instead of [6-chloro-2-(3-propan-2-ylpyrrolidin-1-yppyridin-3-yl]-(1,1-dioxo-1,4-thiazinan-4-yl)methanone. The title compound was obtained in a yield of 53% (65 mg) as a racemic mixture.

1H NMR (400 MHz, CHLOROFORM-d) δ 8.00-7.10 (m, 8 H), 6.24-4.84 (m, 1 H), 4.81-2.57 (m, 8 H), 2.53-2.36 (m, 1 H), 2.35-2.26 (m, 3 H), 2.25-2.02 (m, 1 H), 2.02-1.63 (m, 4 H), 1.62-1.50 (m, 1 H), 1.09 (br d, 2 H), 0.94-0.79 (m, 2 H); LC-MS: 434.2 (MH+).

The racemic mixture (Example 149c) was then separated into single enantiomers by chiral semi-preparative HPLC

| | | |
|---|---|---|
| | | Chiralpak IC (25 × 0.46 cm), 5 u |
| | Column | n-Hexane/Ethanol |
| | Mobile phase | 70/30% v/v |
| | Flow rate (ml/min) | 1 ml/min |
| | DAD detection | 220 nm |
| | Loop | 20 μL |
| | Total amount | 9.7 mg |
| Preparative chiral chromatography protocol: | Injection | 5 mg (each injection) |

| | | | |
|---|---|---|---|
| 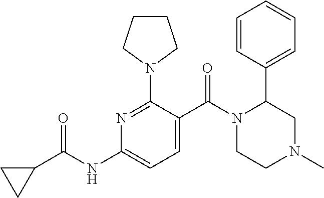 [SMILES: CN1CCN(C(=O)c2ccc(NC(=O)C3CC3)nc2N2CCCC2)C(c2ccccc2)C1] | Enantiomer 1 | Rt 14.4 min | 100% ee |

| 2R or 2S enantiomer | LC-MS: m/z 434.2 (MH+) | | |
|---|---|---|---|
| | (2R or 2S) N-[5-(4-methyl-2-phenylpiperazine-1-carbonyl)- | | |
| | 6-pyrrolidin-1-ylpyridin-2-yl] cyclopropanecarboxamide | | |
| | Example 149a | | |
| 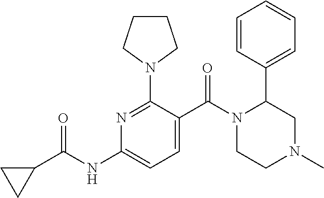 [SMILES: CN1CCN(C(=O)c2ccc(NC(=O)C3CC3)nc2N2CCCC2)C(c2ccccc2)C1] | Enantiomer 2 | Rt 18.7 min | 100% ee |

| | |
|---|---|
| 2R or 2S enantiomer | LC-MS: m/z 434.2 (MH+) |
| | (2R or 2S) N-[5-(4-methyl-2-phenylpiperazine-1-carbonyl)- |
| | 6-pyrrolidin-1-ylpyridin-2-yl] cyclopropanecarboxamide |
| | Example 149b |

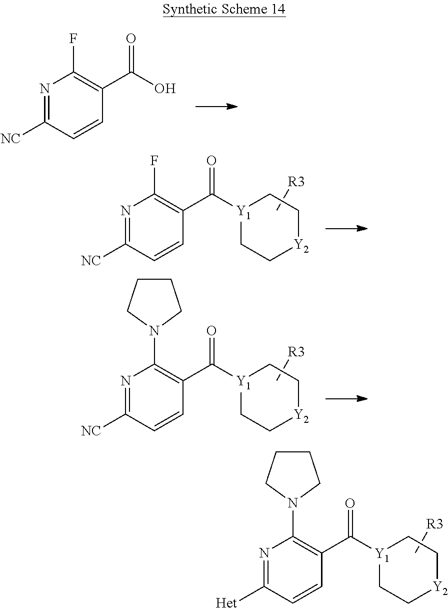

Preparation: 3-fluoro-4-(4-methyl-2-phenylpiperazine-1-carbonyl)benzonitrile

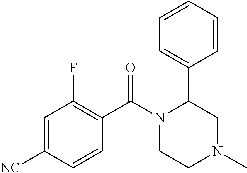 [SMILES: CN1CCN(C(=O)c2ccc(C#N)cc2F)C(c2ccccc2)C1]

A stirred solution of 4-cyano-2-fluorobenzoic acid (500.0 mg, 3.03 mmol), N,N-Diisopropylethylamine (2.64 mL, 15.14 mmol) and HATU: ([dimethylamino(3-triazolo[4,5-b]pyridinyloxy)methylidene]-dimethylammonium hexafluorophosphate (1727.06 mg, 4.54 mmol)) in DMF (15.14 mL) was stirred for 15 minutes at RT. Then 1-methyl-3-phenylpiperazine (640.48 mg, 3.63 mmol) was added and the reaction mixture was stirred at RT for 2 hours. After this time a saturated solution of NaHCO₃ was added to the reaction mixture and the aqueous phase was extracted with AOEt 3 times. The organic portions were collected, washed with Brine, dried over Na₂SO₄ and the solvent was evaporated under reduced pressure. The residue was purified by FC on silica gel (eluting from 100% of cHEx to AcOEt/cHex 1:1) affording the product of formula 3-fluoro-4-(4-methyl-2-phenylpiperazine-1-carbonyl)benzonitrile as a racemic mixture.
Yield: 887 mg
LC-MS: m/z 324.20 (MH+)
   Preparation: 3-fluoro-4-[4-(trifluoromethyppiperidine-1-carbonyl]benzonitrile

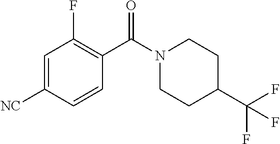 [SMILES: N#Cc1ccc(C(=O)N2CCC(C(F)(F)F)CC2)c(F)c1]

The synthesis of the title compound was effected analogously to the synthesis of 3-fluoro-4-(4-methyl-2-phenylpiperazine-1-carbonyl)benzonitrile wherein 4-(trifluoromethyl)piperidine;hydrochloride was used instead of 1-methyl-3-phenylpiperazine. The title compound was obtained in a yield of 98% (894 mg).
LC-MS: m/z 301.13 (MH+).
   Preparation: 4-(4-methyl-2-phenylpiperazine-1-carbonyl)-3-pyrrolidin-1-ylbenzonitrile

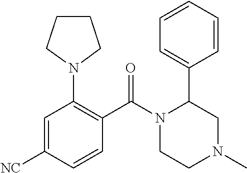 [SMILES: CN1CCN(C(=O)c2ccc(C#N)cc2N2CCCC2)C(c2ccccc2)C1]

A mixture of 3-fluoro-4-(4-methyl-2-phenylpiperazine-1-carbonyl)benzonitrile (887.0 mg, 2.74 mmol) and pyrrolidine (0.68 mL, 8.23 mmol) in MeCN (5.486 mL) was stirred was stirred overnight at RT. The day after the reaction was heated to reflux for 4 hours. After this time the reaction was cooled down to RT and concentrated under vacuum. The residue was purified by FC on NH column (eluting from 100% of cHex to AcOEt/cHex 6:4) affording the product of formula 4-(4-methyl-2-phenylpiperazine-1-carbonyl)-3-pyrrolidin-1-ylbenzonitrile as a racemic mixture.
Yield: 560 mg.

1H NMR (400 MHz, DMSO-d6) δ 7.80-6.86 (m, 8H), 5.88-4.35 (m, 1H), 3.48-3.33 (m, 2H), 3.30-3.20 (m, 1H), 3.16-2.58 (m, 4H), 2.47-2.01 (m, 5H), 2.01-1.86 (m, 3H), 1.81-1.48 (m, 2H), LC-MS: m/z 375.22 (MH+).

Preparation: 3-pyrrolidin-1-yl-4-[4-(trifluoromethyppiperidine-1-carbonyl]benzonitrile

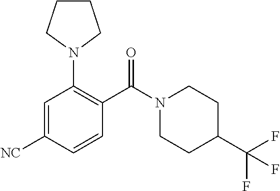 [SMILES: N#Cc1ccc(C(=O)N2CCC(C(F)(F)F)CC2)c(N2CCCC2)c1]

The synthesis of the title compound was effected analogously to the synthesis of 4-(4-methyl-2-phenylpiperazine-1-carbonyl)-3-pyrrolidin-1-ylbenzonitrile wherein 3-fluoro-4-[4-(trifluoromethyppiperidine-1-carbonyl]benzonitrile was used instead of 3-fluoro-4-(4-methyl-2-phenylpiperazine-1-carbonyl)benzonitrile. The title compound was obtained in a yield of 68% (715mg).

1H NMR (400 MHz, DMSO-d6) δ 7.27-6.98 (m, 3H), 4.61 (t, 1H), 3.52 (dd, 1H), 3.30-2.94 (m, 5H), 2.88-2.71 (m, 1H), 2.62 (t, 1H), 1.99-1.83 (m, 5H), 1.76 (d, 1H), 1.41 (dqd, 2H), LC-MS: m/z 352.18 (MH+).

Preparation: (4-methyl-2-phenylpiperazin-1-yl)-[4-(5-methyl-4H-1,2,4-triazol-3-yl)-2-pyrrolidin-1-ylphenyl]methanone

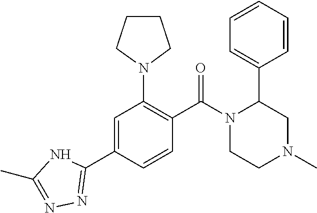

### Example 150a, 150b, 150c

To a stirred solution of 4-(4-methyl-2-phenylpiperazine-1-carbonyl)-3-pyrrolidin-1-ylbenzonitrile (295.0 mg, 0.790 mmol) and N-hydroxyethanimidamide (92.22 mg, 1.18 mmol) in DMSO (2.626 mL), copper diacetate (28.93 mg, 0.160 mmol), dicesium carbonate (774.78 mg, 2.36 mmol) and disodium sulfate (794.39 mg, 5.51 mmol) were added and the reaction mixture was stirred overnight at 120° C. The day after the reaction was cooled down to RT and directly purified by FC on RP using acidic conditions (eluting from CH₃CN/H₂O 5:95 +0.1% of formic acid to CH₃CN/H₂O 6:4 +0.1% of formic acid) and further purified by SCX first washing with MeOH and then with NH₃ 1M in MeOH affording the product of formula (4-methyl-2-phenylpiperazin-1-yl)-[4-(5-methyl-4H-1,2,4-triazol-3-yl)-2-pyrrolidin-1-ylphenyl]methanone as a racemic mixture.
Yield: 9.1 mg.

1H NMR (400 MHz, DMSO-d6) δ 13.61 (s, 1H), 8.03-6.72 (m, 8H), 5.91-4.61 (m, 1H), 4.55-3.40 (m, 3H), 3.32 (d, 3H), 3.15-2.60 (m, 4H), 2.46-2.12 (m, 6H), 1.96 (s, 2H), 1.80-1.60 (m, 2H); LC-MS: m/z 431.25 (MH+).

The racemic mixture (Example 150c) was then separated into single enantiomers by chiral semi-preparative HPLC

| | Column | Whelk O1 (R,R) (25 × 3.0 cm), 10μ | |
|---|---|---|---|
| | phase | n-Hexane/Ethanol 70/30% v/v | |
| | Flow rate (ml/min) | 40 ml/min | |
| | DAD detection | 254 nm | |
| Preparative chiral | Loop | 750 μL | |
| chromatography | Total amount | 5.5 mg | |
| protocol: | Injection | 2.7 mg (each injection) | |
| 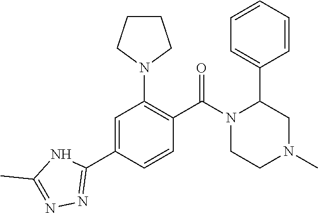 | Enantiomer 1 | Rt 22.46 min | 100% ee |

| 2R or 2S enantiomer | LC-MS: m/z 431.25 (MH+) | | |
|---|---|---|---|
| | (2R or 2S) (4-methyl-2-phenylpiperazin-1-yl)-[4-(5-methyl- | | |
| | 4H-1,2,4-triazol-3-yl)-2-pyrrolidin-1-ylphenyl]methanone | | |
| | Example 150a | | |
| 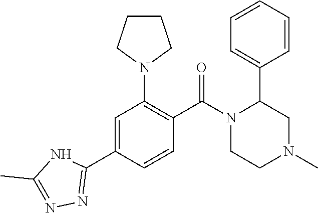 | Enantiomer 2 | Rt 29.62 min | 100% ee |

| | |
|---|---|
| 2R or 2S enantiomer | LC-MS: m/z 431.25 (MH+) |
| | (2R or 2S) (4-methyl-2-phenylpiperazin-1-yl)-[4-(5-methyl- |
| | 4H-1,2,4-triazol-3-yl)-2-pyrrolidin-1-ylphenyl]methanone |
| | Example 150b |

Preparation: [4-(5-cyclopropyl-4H-1,2,4-triazol-3-yl)-2-pyrrolidin-1-yl phenyl]-(4-methyl-2phenylpiperazin-1-yl)methanone

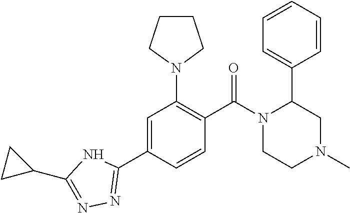 [SMILES: CN1CCN(C(=O)c2ccc(-c3nnc(C4CC4)[nH]3)cc2N2CCCC2)C(c2ccccc2)C1]

### Example 151a, 151b, 151c

The synthesis of the title compound was effected analogously to the synthesis of the compound of Example 150 a, b ((4-methyl-2-phenylpiperazin-1-yl)-[4-(5-methyl-4H-1,2,4-triazol-3-yl)-2-pyrrolidin-1-ylphenyl]methanone) wherein N-hydroxycyclopropanecarboximidamide was used instead of N-hydroxyethanimidamide. The title compound was obtained in a yield of 10% (30 mg) as a racemic mixture.

1H NMR (600 MHz, DMSO-d6) δ 13.74 (br s, 1 H), 7.83-7.44 (m, 2 H), 7.43-7.09 (m, 5 H), 7.16-6.93 (m, 1 H), 5.95-4.62 (m, 1 H), 4.73-3.20 (m, 1 H), 3.35-2.23 (m, 9 H), 2.27-2.12 (m, 3 H), 2.10-1.98 (m, 1 H), 1.98-1.51 (m, 4 H), 1.08-0.76 (m, 4 H), LC-MS: m/z 457.27 (MH+).

The racemic mixture (Example 151c) was then separated into single enantiomers by chiral semi-preparative HPLC

| | Column | Whelk O1 (R,R) (25 × 3.0 cm), 10μ | |
|---|---|---|---|
| | phase | n-Hexane/Ethanol 70/30 % v/v | |
| | Flow rate (ml/min) | 40 ml/min | |
| | DAD detection | 220 nm | |
| Preparative chiral | Loop | 1000 μL | |
| chromatography | Total amount | 18.5 mg | |
| protocol: | Injection | 5 mg (each injection) | |
| 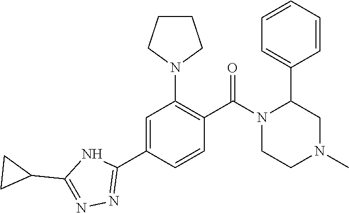 [SMILES: CN1CCN(C(=O)c2ccc(-c3nnc(C4CC4)[nH]3)cc2N2CCCC2)C(c2ccccc2)C1] | Enantiomer 1 | Rt 18.17 min | 100% ee |
| 2R or 2S enantiomer | LC-MS: m/z 457.27 (MH+) | | |
| | (2R or 2S) [4-(5-cyclopropyl-4H-1,2,4-triazol-3-yl)-2-pyrrolidin- | | |
| | 1-ylphenyl]-(4-methyl-2-phenylpiperazin-1-yl)methanone | | |
| | Example 151a | | |
| 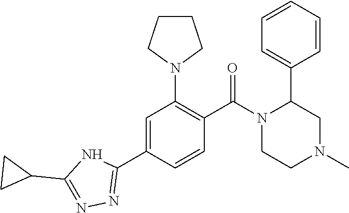 [SMILES: CN1CCN(C(=O)c2ccc(-c3nnc(C4CC4)[nH]3)cc2N2CCCC2)C(c2ccccc2)C1] | Enantiomer 2 | Rt 24.38 min | 100% ee |

| | |
|---|---|
| 2R or 2S enantiomer | LC-MS: m/z 457.27 (MH+) |
| | (2R or 2S) [4-(5-cyclopropyl-4H-1,2,4-triazol-3-yl)-2-pyrrolidin-1- |
| | ylphenyl]-(4-methyl-2-phenylpiperazin-1-yl)methanone |
| | Example 151b |

Preparation: [4-(3-methyl-1,2,4-oxadiazol-5-yl)-2-pyrrolidin-1-ylphenyl]-[4-(trifluoromethyppiperidin yl]methanone

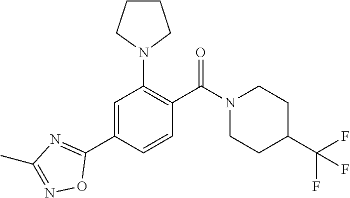 [SMILES: Cc1noc(-c2ccc(C(=O)N3CCC(C(F)(F)F)CC3)c(N3CCCC3)c2)n1]

### Example 152

To a mixture of 3-pyrrolidin-1-yl-4-[4-(trifluoromethyppiperidine-1-carbonyl]benzonitrile (100.0 mg, 0.280 mmol) and N-hydroxyethanimidamide (33.32 mg, 0.430 mmol) in DMF (0.569 mL) under nitrogen atmosphere, anhydrous zinc chloride (11.81 mg, 0.090 mmol) and Trifluoroacetic acid (0.01 mL, 0.090 mmol) were added under N₂. The reaction mixture was heated under MW irradiation at 120° C. for 30 minutes. After this time a saturated solution of NaHCO₃ was added to the mixture and the aqueous phase was extracted with EtOAc 3 times. The organic portions were collected, washed with Brine, dried over Na₂SO₄ and the solvent was evaporated under reduced pressure. The residue was purified by FC on RP using acidic conditions (eluting from CH₃CN/H₂O 5:95 +0.1% of formic acid to CH₃CN/H₂O 8:2 +0.1% of formic acid) affording the product of formula [4-(3-methyl-1,2,4-oxadiazol-5-yl)-2-pyrrolidin-1-ylphenyl]-[4-(trifluoromethyl)piperidin-1-yl]methanone.
Yield: 10.4 mg

1H NMR (500 MHz, DMSO-d6) δ ppm 7.54-7.42 (m, 2 H), 7.37-7.19 (m, 1 H), 4.84-4.73 (m, 1 H), 3.80-3.63 (m, 1 H), 3.42-3.34 (m, 2 H), 3.26-2.78 (m, 4 H), 2.60-2.47 (m, 1 H), 2.44 (s, 3 H), 2.13-1.42 (m, 8 H); LC-MS: m/z 409.19 (MH+).

Preparation: [4-(5-methyl-4H-1,2,4-triazol-3-yl)-2-pyrrolidin-1-ylphenyl]-[4-(trifluoromethyl)piperidin-1-yl]methanone

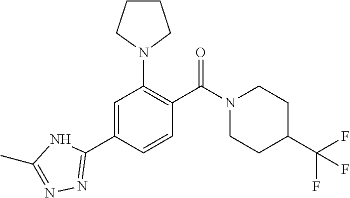 [SMILES: Cc1nnc(-c2ccc(C(=O)N3CCC(C(F)(F)F)CC3)c(N3CCCC3)c2)[nH]1]

### Example 153

To a stirred solution of 3-pyrrolidin-1-yl-4-[4-(trifluoromethyppiperidine-1-carbonyl]benzonitrile (100.0 mg, 0.280 mmol) and N-hydroxyethanimidamide (33.32 mg, 0.430 mmol) in DMSO (0.949 mL), copper diacetate (10.45 mg, 0.060 mmol), dicesium carbonate (279.91 mg, 0.850 mmol) and disodium sulfate (287.0 mg, 1.99 mmol) were added and the reaction mixture was stirred overnight at 120° C. The day after, further N-hydroxyethanimidamide (33.32 mg, 0.430 mmol) and copper diacetate (10.45 mg, 0.060 mmol) were added and the reaction was stirred at 120° C. for additional 12 h. The day after the reaction was cooled down to RT and directly purified by FC on RP using acidic conditions (eluting from CH₃CN/H₂O 5:95 +0.1% of formic acid to CH₃CN/H₂O 6:4 +0.1% of formic acid) affording the product of formula [4-(5-methyl-4H-1,2,4-triazol-3-yl)-2-pyrrolidin-1-ylphenyl]-[4-(trifluoromethyl)piperidin-1-yl]methanone.
Yield: 5.7 mg

1H NMR (400 MHz, DMSO-d6) δ 13.73 (s, 1H), 7.43-7.23 (m, 2H), 7.09 (dd, 1H), 4.64 (t, 1H), 3.60 (d, 1H), 3.28-2.93 (m, 5H), 2.89-2.65 (m, 2H), 2.38 (s, 3H), 1.90 (s, 5H), 1.77 (d, 1H), 1.39 (dq, 2H); LC-MS: m/z 408.18 (MH+).

Preparation: [4-(5-cyclopropyl-4H-1,2,4-triazol-3-y0-2-pyrrolidin-1-ylphenyl]-[4-(trifluoromethyl) piperidin-1-yl]methanone

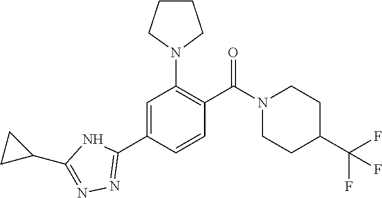 [SMILES: O=C(c1ccc(-c2nnc(C3CC3)[nH]2)cc1N1CCCC1)N1CCC(C(F)(F)F)CC1]

### Example 154

The synthesis of the title compound was effected analogously to the synthesis of the compound of Example 153 ([4-(5-methyl-4H-1,2,4-triazol-3-yl)-2-pyrrolidin-1-ylphenyl]-[(4-(trifluoromethyppiperidin-1-yl] methanone) wherein N-hydroxycyclopropanecarboximidamide was used instead of N-hydroxyethanimidamide. The title compound was obtained in a yield of 6% (6.7 mg).

1H NMR (500 MHz, DMSO-d6) δ ppm 14.40-13.26 (m, 1 H), 7.97-6.54 (m, 3 H), 4.76-4.53 (m, 1 H), 3.76-3.45 (m, 1 H), 3.29-2.91 (m, 5 H), 2.83-2.68 (m, 1 H), 2.66-2.56 (m, 1 H), 2.16-1.99 (m, 1 H), 1.97-1.67 (m, 6 H), 1.50-1.28 (m, 2 H), 1.15-0.83 (m, 4 H), LC-MS: m/z 434.21 (MH+).

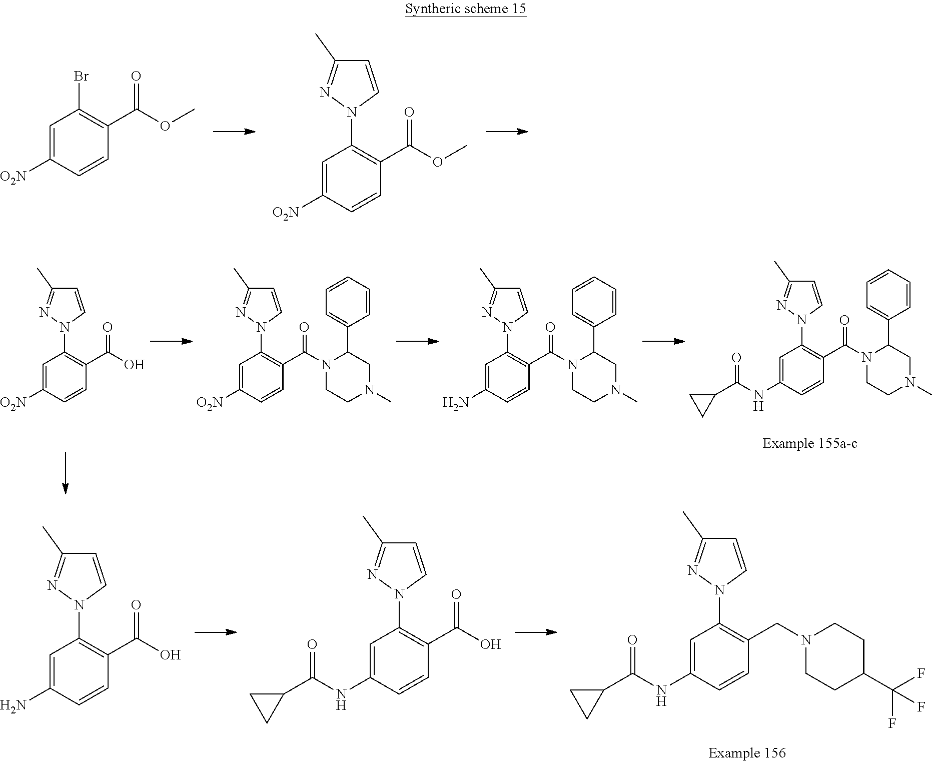

Preparation: methyl 2-(3-methylpyrazol-1-yl)-4-nitrobenzoate

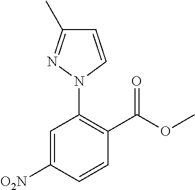 [SMILES: COC(=O)c1ccc([N+](=O)[O-])cc1-n1ccc(C)n1]

A suspension of methyl 2-bromo-4-nitrobenzoato (2 g, 7.69 mmol), 3-methyl-1H-pyrazole 3 (1.24 mL, 15.38 mmol), dicesium carbonate (5042.92 mg, 15.38 mmol), N,N′-Dimethylcyclohexane-1,2-diamine (0.24 mL, 1.54 mmol) and copper (I) iodide (73.63 mg, 0.380 mmol) in a mixture of 1,4-Dioxane (12.6 mL)/ Water (0.200 mL) was degassed with Schelnk line technique and heated to 100° C. for 4 hours. After this time the reaction mixture was diluted with H₂O and brought to pH 2 with HCI 3N and the mixture was extracted with AcOEt 3 times. The combined organic fractions were washed with Brine, dried over Na₂SO₄, filtered and concentrated under reduced pressure affording the product of formula methyl 2-(3-methylpyrazol-1-yl)-4-nitrobenzoate, which was used in the next step without further purification
Yield: 1.8 g
LC-MS: m/z 262.08 (MH+)
   Preparation: 2-(3-methylpyrazol-1-yl)-4-nitrobenzoic acid

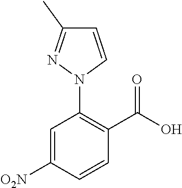 [SMILES: Cc1ccn(-c2cc([N+](=O)[O-])ccc2C(=O)O)n1]

To a solution of methyl 2-(3-methylpyrazol-1-yl)-4-nitrobenzoate (1.8 g, 6.89 mmol) in THF (8.05 mL) and Methanol (2.68 mL), a solution of lithium hydroxide (384.38 mg, 15.4 mmol) in Water (2.68 mL) was added. The reaction was stirred at 50° C. overnight. The day after the reaction was cooled down to RT and brought to pH 2 with HCI 3N and the mixture was extracted with AcOEt 3 times. The combined organic fractions were then washed with Brine, dried over Na₂SO₄, filtered and concentrated under vacuum. The raw material was purified by Semi preparative MDAP Method:

| | | |
|---|---|---|
| | Column: | XSelect CSH Prep. C18 5 μm OBD 30 × 100 mm @ room T |
| | Solvents: | A = H₂O + 0.1% HCOOH B = Acetonitrile |
| | Method: | from 3% B to 99.9% B in 10 min |
| Preparative achiral | Injection loop: | 1 mL (each injection) |
| chromatography | Flow rate (ml/min): | 40 ml/min |
| protocol: | UV detection range: | 210 nm to 350 nm |

| | |
|---|---|
| 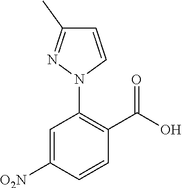 [SMILES: Cc1ccn(-c2cc([N+](=O)[O-])ccc2C(=O)O)n1] | 2-(3-methylpyrazol-1-yl)-4-nitrobenzoic acid Yield: 20% (340 mg) 1H NMR (400 MHz, DMSO-d6 ) δ ppm 13.56 (br s, 1 H), 8.35 (d, 1 H), 8.22-8.20 (m, 1 H), 8.24-8.19 (m, 1 H), 7.89 (d, 1 H), 6.36 (d, 1 H), 2.25 (s, 3 H); LC-MS: m/z 248.1 (MH+). |

Preparation: (4-methyl-2-phenylpiperazin-1-yl)-[2-(3-methylpyrazol-1-yl)-4-nitrophenyl]methanone

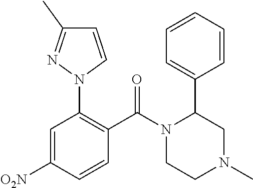 [SMILES: Cc1ccn(-c2cc([N+](=O)[O-])ccc2C(=O)N2CCN(C)CC2c2ccccc2)n1]

To a solution of 2-(3-methylpyrazol-1-yl )-4-nitrobenzoic acid (60.0 mg, 0.240 mmol) in DMF (3 mL) and N,N-Diisopropylethylamine (78.42 mg, 0.610 mmol), HATU: [dimethylamino(3-triazolo[4,5-b]pyridinyloxy)methylidene]-dimethylammonium hexafluorophosphate (0.12 mL, 0.240 mmol) was added and the mixture was stirred at rt for 30 min. After this time 1-methyl-3-phenylpiperazine (49.2 mg, 0.280 mmol) was added and the reaction was stirred at room temperature ON. The day after the reaction mixture was diluted with water and extracted with AcOEt 3 times. The combined organic fractions were washed with Brine, dried over Na₂SO₄, filtered and concentrated under reduced pressure affording the product of formula (4-methyl-2-phenylpiperazin-1-yl)-[2-(3-methylpyrazol-1-yl)-4-nitrophenyl]methanone, as a racemic mixture. The product was used in the next step without further purification.
Yield: 100 mg
LC-MS: m/z 406.5 (MH+)
   Preparation: [4-amino-2-(3-methylpyrazol-1-yl)phenyl]-(4-methyl-2-phenylpiperazin-1-yl)methanone

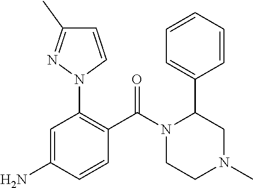 [SMILES: Cc1ccn(-c2cc(N)ccc2C(=O)N2CCN(C)CC2c2ccccc2)n1]

To a solution of (4-methyl-2-phenylpiperazin-1-yl)-[2-(3-methylpyrazol-1-yl)-4-nitrophenyl]methanone (100.0 mg, 0.250 mmol) in Methanol (2.466 mL) was added Pd-C 10% (26.25 mg, 0.020 mmol). The resulting solution was stirred at RT for 1 h under H₂ atmosphere (1 atm). After this time the reaction mixture was filtered through a pad of celite and concentrated under reduced pressure affording the product of formula [4-amino-2-(3-methylpyrazol-1-yl)phenyl]-(4-methyl-2-phenylpiperazin-1-yl)methanone, which was used in the next step without further purification.
Yield: 69 mg
LC-MS: m/z 376.1 (MH+).
   Preparation: N-[4-(4-methyl-2-phenylpiperazine-1-carbonyl)-3-(3-methylpyrazol-1-yl)phenyl]cyclopropanecarboxamide

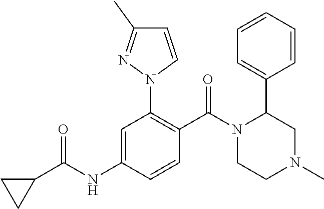 [SMILES: Cc1ccn(-c2cc(NC(=O)C3CC3)ccc2C(=O)N2CCN(C)CC2c2ccccc2)n1]

### Example 155a, 155b, 155c

To a solution of [4-amino-2-(3-methylpyrazol-1-yl)phenyl]-(4-methyl-2-phenylpiperazin-1-yl)methanone (68.9 mg, 0.180 mmol) (0.918 mL) and triethylamine (0.08 mL, 0.550 mmol) cyclopropanecarbonyl chloride (0.04 mL, 0.460 mmol) was added and the reaction was stirred ON at rt. The day after the reaction mixture was diluted with DCM and washed with s. s. of NaHCO₃, dried through a phase separator and concentrated under reduced pressure. The raw material was purified by FC on RP using basic conditions (eluting from H₂O +0.1% of ammonium hydroxide/ CH₃CN 95:5 to H₂O +0.1% of ammonium hydroxide/CH₃CN 5:95) affording the product of formula N-[4-(4-methyl-2-phenylpiperazine-1-carbonyl)-3-(3-methylpyrazol-1-yl)phenyl]cyclopropanecarboxamide as a racemic mixture.
Yield: 27.5 mg

1H NMR (500 MHz, DMSO-d6) δ 10.59-10.44 (m, 1 H), 7.89 (br d, 1 H), 7.88-7.67 (m, 1 H), 7.65-7.51(m, 1 H), 7.47-7.19 (m, 6 H), 6.45-6.07 (m, 1 H), 5.78-4.38 (m, 1 H), 2.32-2.15 (m, 3 H), 2.20-2.00 (m, 3 H), 1.83-1.71 (m, 1 H), 4.36-1.17 (m, 6 H), 0.87-0.76 (m, 4 H); LC-MS: m/z 444.3 (MH+).

The racemic mixture (Example 155c) was then separated into single enantiomers by chiral semi-preparative HPL

| | | |
|---|---|---|
| | Column | Chiralpak AS-H (25 × 2.0 cm), 5μ |
| | Mobile phase | n-Hexane/Ethanol 85/15% v/v |
| | Flow rate (ml/min) | 17 ml/min |
| | DAD detection | 220 nm |
| Preparative chiral | Loop | 1000 μL |
| chromatography | Total amount | 24.6 mg |
| protocol: | Injection | 9.1 mg (each injection) |

| 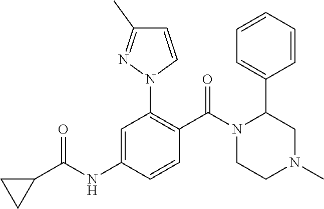 [SMILES: Cc1ccn(-c2cc(NC(=O)C3CC3)ccc2C(=O)N2CCN(C)CC2c2ccccc2)n1] | Enantiomer 1 | Rt 6.35 min | 100% ee |
|---|---|---|---|
| 2R or 2S enantiomer | LC-MS: m/z 444.3 (MH+) | | |
| | (2R or 2S) N-[4-(4-methyl-2-phenylpiperazine-1-carbonyl)- | | |
| | 3-(3-methylpyrazol-1-yl)phenyl] cyclopropanecarboxamide | | |
| | Example 155a | | |
| 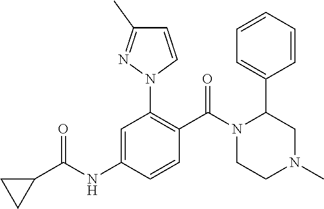 [SMILES: Cc1ccn(-c2cc(NC(=O)C3CC3)ccc2C(=O)N2CCN(C)CC2c2ccccc2)n1] | Enantiomer 2 | Rt 11.88 min | 100% ee |

| | |
|---|---|
| 2R or 2S enantiomer | LC-MS: m/z 444.3 (MH+) |
| | (2R or 2S) N-[4-(4-methyl-2-phenylpiperazine-1-carbonyl)- |
| | 3-(3-methylpyrazol-1-yl)phenyl] cyclopropanecarboxamide |
| | Example 155b |

Preparation: 4-amino-2-(3-methylpyrazol-1-yl)benzoic acid

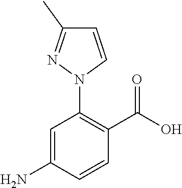 [SMILES: Cc1ccn(-c2cc(N)ccc2C(=O)O)n1]

To a solution of solution 2-(3-methylpyrazol-1-yl)-4-nitrobenzoic acid (340.0 mg, 1.38 mmol) in Methanol (25 mL) was added palladium (146.36 mg, 0.140 mmol) on carbon (10% w/w) and stirred under H₂ atmosphere for 3h. After this time the reaction mixture was filtered through a pad of celite and washed with methanol. The organic phase was concentrated under reduced pressure affording the product of formula 4-amino-2-(3-methylpyrazol-1-yl )benzoic acid, which was used in the next step without further purification.
Yield: 290 mg

1H NMR (400 MHz, CHLOROFORM-d) δ 8.10 (d, 1 H), 7.63-7.80 (m, 1 H), 6.64-6.77 (m, 1 H), 6.48-6.60 (m, 1 H), 6.28-6.37 (m, 1 H), 3.51 (s, 2 H), 2.41 (s, 3 H); LC-MS: m/z 218.0 (MH+)

Preparation: 4-(cyclopropanecarbonylamino)-2-(3-methylpyrazol-1-yl)benzoic acid

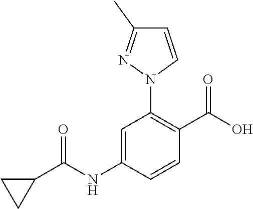 [SMILES: Cc1ccn(-c2cc(NC(=O)C3CC3)ccc2C(=O)O)n1]

A solution of cyclopropanecarboxylic acid (0.11 mL, 1.34 mmol), HATU:[dimethylamino(3-triazolo[4,5-b]pyridinyloxy)methylidene]dimethylammonium hexafluorophosphate (507.63 mg, 1.34 mmol) and N,N-Diisopropylethylamine (0.58 mL, 3.34 mmol) in DMF (8.35 mL) was stirred at RT for 45 min. After this time, at this solution 4-amino-2-(3-methylpyrazol-1-yl)benzoic acid (290.0 mg, 1.34 mmol) dissolved in DMF (5 mL) was added and the resulting mixture was stirred ON at RT. The day after the reaction mixture was diluted with H₂On the pH was adjusted to pH=10 with NaOH 3M and the reaction was washed with DCM. The aqueous solution was then acidified until pH =2 with HCI 3 N and it was extracted with AcOEt 3 times. The combined organic fractions were washed with Brine, dired over Na₂SO₄, filtered and concentrated under reduced pressure affording the product of formula 4-(cyclopropanecarbonylamino)-2-(3-methylpyrazol-1-yl)benzoic acid, which was used in the next step without further purification.
Yield: 340 mg
LC-MS: m/z 286.10 (MH+)
   Preparation: N-[3-(3-methylpyrazol-1-yl)-4-[4-(trifluoromethyppiperidine-1-carbonyl]phenyl]cyclopropanecarboxamide

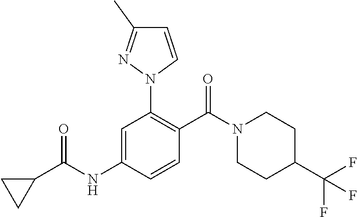 [SMILES: Cc1ccn(-c2cc(NC(=O)C3CC3)ccc2C(=O)N2CCC(C(F)(F)F)CC2)n1]

### Example 156

The synthesis of the title compound was effected analogously to the synthesis of (4-methyl-2-phenylpiperazin-1-yl )-[2-(3-methylpyrazol-1-yl)-4-nitrophenyl]methanone wherein 4-(cyclopropanecarbonylamino)-2-(3-methylpyrazol-1-yl )benzoic acid and 4-(trifluoromethyl)piperidine hydrochloride were used instead of 2-(3-methylpyrazol-1-yl )-4-nitrobenzoic acid and 1-methyl-3-phenylpiperazine respectively. The title compound was obtained in a yield of 3.2% (16 mg).

1H NMR (400 MHz, DMSO-d6) δ ppm 10.52 (s, 1 H), 7.98-7.87 (m, 1 H), 7.87-7.75 (m, 1 H), 7.55 (dd, 1 H), 7.40-7.19 (m, 1 H), 6.29 (d, 1 H), 4.66-3.32 (m, 2 H), 3.08-2.62 (m, 2 H), 2.60-2.49 (m, 1 H), 2.28-2.16 (m, 3 H), 1.83-1.74 (m, 1 H), 0.86-0.80 (m, 4 H), 1.93-0.68 (m, 4 H); LC-MS: m/z 421.1 (MH+).

Preparation: N-[4-(4-propylpiperazine-1-carbonyl)-3-[3-(trifluoromethyl)pyrazol-1-yl]phenyl]cyclopropanecarboxamide

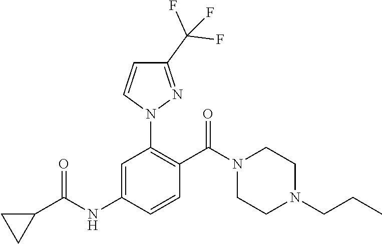 [SMILES: CCCN1CCN(C(=O)c2ccc(NC(=O)C3CC3)cc2-n2ccc(C(F)(F)F)n2)CC1]

### Example 158

In a vial N-[3-bromo-4-(4-propylpiperazine-1-carbonyl)phenyl]cyclopropanecarboxamide (150.0 mg, 0.380 mmol), tripotassium phosphate (171.99 mg, 0.800 mmol), 3-(trifluoromethyl)pyrazole (62.12 mg, 0.460 mmol) were suspended in dry 1,4-Dioxane (1.869 mL). The resulting suspension was degassed bubbling N2 for 15 minutes, then copper (I) iodide (3.64 mg, 0.020 mmol) and N,N′-Dimethylcyclohexane-1,2-diamine (6.0 uL, 0.040 mmol) were added under nitrogen flux and degassed again with Schlenk line technique. The vial was sealed and stirred for 48 h at 100° C. The day after the reaction mixture was diluted with AcOEt and filtered through a pad of celite. The organic layer was washed with brine, dried over Na₂SO₄, filtered and the solvent removed under reduced pressure. The residue was purified by FC on RP using acid conditions (eluting from 5:95 of CH₃CN/H₂O +0.1% of formic acid to 30:70 of CH₃CN/H₂O +1% of FA) affording the product of formula N-[4-(4-propylpiperazine-1-carbonyl)-3-[3-(trifluoromethyl)pyrazol-1-yl]phenyl]cyclopropanecarboxamide.
Yield: 69 mg

1H NMR (400 MHz, DMSO) δ 10.62 (s, 1H), 8.21 (dd, J=2.5, 1.1 Hz, 1H), 8.00 (d, J=2.0 Hz, 1H), 7.67 (dd, J=8.4, 2.0 Hz, 1H), 7.38 (d, J=8.4 Hz, 1H), 6.99 (d, J=2.5 Hz, 1H), 3.62 (s, 2H), 3.17-2.90 (m, 2H), 2.33 (d, J=60.4 Hz, 2H), 2.20-2.02 (m, 3H), 1.90-1.73 (m, 2H), 1.38 (h, J=7.4 Hz, 2H), 0.92-0.77 (m, 7H); LC-MS: m/z 450.2.

Preparation: tert-butyl 3-(3-chlorophenyl)-4-[4-(cyclopropanecarbonylamino)-2-pyrrolidin-1-ylbenzoyl]piperazine-1-carboxylate

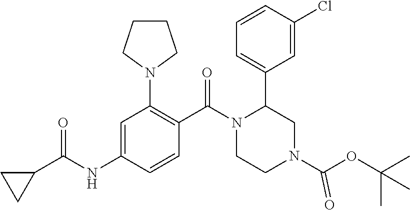 [SMILES: CC(C)(C)OC(=O)N1CCN(C(=O)c2ccc(NC(=O)C3CC3)cc2N2CCCC2)C(c2cccc(Cl)c2)C1]

### Example 159

The synthesis of the title compound was effected analogously to the synthesis of the compound of Example 136 (tert-butyl 4-[4-(cyclopropanecarbonylamino)-2-pyrrolidin-1-ylbenzoyI]-3-(4-fluorophenyl)piperazine-1-carboxylate) wherein tert-butyl 3-(3-chlorophenyl)piperazine-1-carboxylate was used instead of tert-butyl 3-(4-fluorophenyl)piperazine-1-carboxylate. The title compound was obtained in a yield of 55% (67 mg) as a racemic mixture.
LC-MS: m/z 553.30 (MH+)
   Preparation: tert-butyl 3-(2-chlorophenyl)-4-[4-(cyclopropanecarbonylamino)-2-pyrrolidin-1-ylbenzoyl]piperazine-1-carboxylate

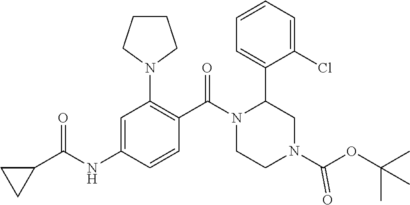 [SMILES: CC(C)(C)OC(=O)N1CCN(C(=O)c2ccc(NC(=O)C3CC3)cc2N2CCCC2)C(c2ccccc2Cl)C1]

### Example 160

The synthesis of the title compound was effected analogously to the synthesis of the compound of Example 136 (tert-butyl 4-[4-(cyclopropanecarbonylamino)-2-pyrrolidin-1-ylbenzoyI]-3-(4-fluorophenyl)piperazine-1-carboxylate) wherein tert-butyl 3-(2-chlorophenyl)piperazine-1-carboxylate was used instead of tert-butyl 3-(4-fluorophenyl)piperazine-1-carboxylate. The title compound was obtained in a yield of 37% (45 mg) as a racemic mixture.
LC-MS: m/z 553.27 (MH+)
   Preparation: tert-butyl 3-(5-chloropyridin-3-yl)-4-[4-(cyclopropanecarbonylamino)-2-pyrrolidin-1-ylbenzoyl]piperazine-1-carboxylate

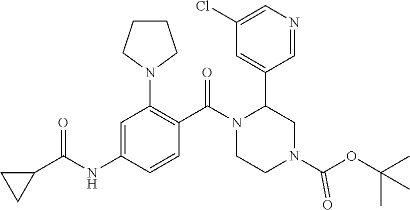 [SMILES: CC(C)(C)OC(=O)N1CCN(C(=O)c2ccc(NC(=O)C3CC3)cc2N2CCCC2)C(c2cncc(Cl)c2)C1]

### Example 161

The synthesis of the title compound was effected analogously to the synthesis of the compound of Example 136 (tert-butyl 4-[4-(cyclopropanecarbonylamino)-2-pyrrolidin-1-ylbenzoyI]-3-(4-fluorophenyl)piperazine-1-carboxylate) wherein tert-butyl 3-(5-chloropyridin-3-yl)piperazine-1-carboxylate was used instead of tert-butyl 3-(4-fluorophenyl)piperazine-1-carboxylate. The title compound was obtained in a yield of 44% (55 mg) as a racemic mixture.
LC-MS: m/z 554.27 (MH+).
   Preparation: tert-butyl 4[4-(cyclopropanecarbonylamino)-2-pyrrolidin-1-ylbenzoyl]-3-pyridin-3-ylpiperazine-1-carboxylate

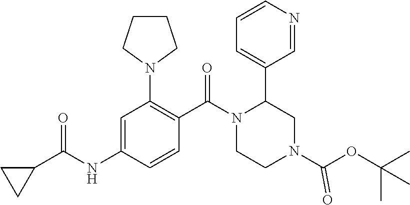 [SMILES: CC(C)(C)OC(=O)N1CCN(C(=O)c2ccc(NC(=O)C3CC3)cc2N2CCCC2)C(c2cccnc2)C1]

### Example 162

The synthesis of the title compound was effected analogously to the synthesis of the compound of Example 136 (tert-butyl 4-[4-(cyclopropanecarbonylamino)-2-pyrrolidin-1-ylbenzoyI]-3-(4-fluorophenyl)piperazine-1-carboxylate) wherein tert-butyl 3-pyridin-3-ylpiperazine-1-carboxylate was used instead of tert-butyl 3-(4-fluorophenyl)piperazine-1-carboxylate. The title compound was obtained in a yield of 56% (64 mg) as a racemic mixture.
LC-MS: m/z 520.26 (MH+).
   Preparation: tert-butyl 3-(3-chloropyridin-4-yl)-4-[4-(cyclopropanecarbonylamino)-2-pyrrolidin-1-ylbenzoyl]piperazine-1-carboxylate

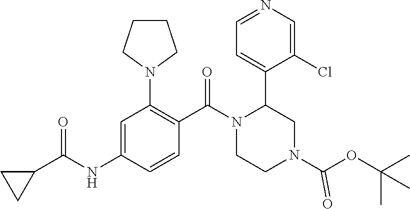 [SMILES: CC(C)(C)OC(=O)N1CCN(C(=O)c2ccc(NC(=O)C3CC3)cc2N2CCCC2)C(c2ccncc2Cl)C1]

### Example 163

The synthesis of the title compound was effected analogously to the synthesis of the compound of Example 136 (tert-butyl 4-[4-(cyclopropanecarbonylamino)-2-pyrrolidin-1-ylbenzoyI]-3-(4-fluorophenyl)piperazine-1-carboxylate) wherein tert-butyl 3-(3-chloropyridin-4-yl)piperazine-1-carboxylate was used instead of tert-butyl 3-(4-fluorophenyl)piperazine-1-carboxylate. The title compound was obtained in a yield of 20% (25 mg) as a racemic mixture.
LC-MS: m/z 554.26 (MH+).
   Preparation: tert-butyl 4-[4-(cyclopropanecarbonylamino)-2-pyrrolidin-1-ylbenzoyl]-3-pyrimidin-5-ylpiperazine-1-carboxylate

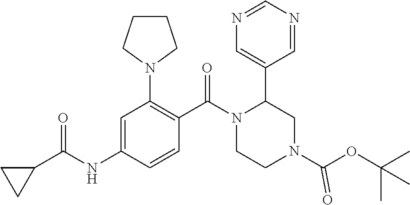 [SMILES: CC(C)(C)OC(=O)N1CCN(C(=O)c2ccc(NC(=O)C3CC3)cc2N2CCCC2)C(c2cncnc2)C1]

### Example 164

The synthesis of the title compound was effected analogously to the synthesis of the compound of Example 136 (tert-butyl 4-[4-(cyclopropanecarbonylamino)-2-pyrrolidin-1-ylbenzoyI]-3-(4-fluorophenyl)piperazine-1-carboxylate) wherein tert-butyl 3-pyrimidin-5-ylpiperazine-1-carboxylate was used instead of tert-butyl 3-(4-fluorophenyl)piperazine-1-carboxylate. The title compound was obtained in a yield of 46% (52 mg) as a racemic mixture.
LC-MS: m/z 521.28 (MH+).
   Preparation: tert-butyl 3-(4-chlorophenyl)-4-[4-(cyclopropanecarbonylamino)-2-pyrrolidin-1-ylbenzoyl]piperazine-1-carboxylate

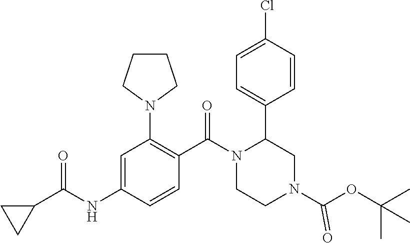 [SMILES: CC(C)(C)OC(=O)N1CCN(C(=O)c2ccc(NC(=O)C3CC3)cc2N2CCCC2)C(c2ccc(Cl)cc2)C1]

### Example 165

The synthesis of the title compound was effected analogously to the synthesis of the compound of Example 136 (tert-butyl 4-[4-(cyclopropanecarbonylamino)-2-pyrrolidin-1-ylbenzoyI]-3-(4-fluorophenyl)piperazine-1-carboxylate) wherein tert-butyl 3-(4-chlorophenyl)piperazine-1-carboxylate was used instead of tert-butyl 3-(4-fluorophenyl)piperazine-1-carboxylate. The title compound was obtained in a yield of 43% (52 mg) as a racemic mixture.

Preparation: N-[3-pyrrolidin-1-yl-4-[2-(trifluoromethyl)morpholine-4-carbonyl]phenyl]cyclopropanecarboxamide

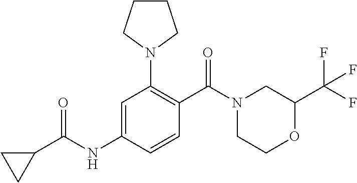 [SMILES: O=C(Nc1ccc(C(=O)N2CCOC(C(F)(F)F)C2)c(N2CCCC2)c1)C1CC1]

### Example 166

To A stirred solution of 4-(cyclopropanecarbonylamino)-2-pyrrolidin-1-ylbenzoic acid (50.0 mg, 0.180 mmol), and HATU ([dimethylamino(3-triazolo[4,5-b]pyridinyloxy)methylidene]-dimethylammonium;hexafluorophosphate (100.84 mg, 0.270 mmol)) in DMF (1.13 mL) N,N-Diisopropylethylamine (0.18 mL, 1.06 mmol) was added and the mixture was stirred for 15 minutes at RT. After this time a solution of 2-(trifluoromethyl)morpholine hydrochloride (40.65 mg, 0.210 mmol) and Diisopropylethylamine (0.09 mL, 0.53 mmol) in (DMF 1 mL) was added and the reaction mixture was stirred at RT for 8 hours. A saturated solution of NaHCO₃ was added to the reaction mixture and the aqueous phase was extracted with EtOAc (x3). The organic portions were collected, washed with brine, dried over Na₂SO₄, filtered and concentrated under vacuum. The resude was purified by FC on silica gel (eluting from 100% of DCM to DCM/AcOEt 70/30) affording the product of formula N-[3-pyrrolidin-1-yl-4-[2-(trifluoromethyl)morpholine-4-carbonyl]phenyl]cyclopropanecarboxamide.
Yield: 50 mg.

1H NMR (400 MHz, DMSO-d6) δ 10.13 (d, J=14.3 Hz, 1H), 7.22-6.86 (m, 3H), 4.56-3.81 (m, 3H), 3.71-3.45 (m, 2H), 3.15 (dd, J=14.4, 7.1 Hz, 4H), 2.97 (d, J=28.1 Hz, 2H), 1.89 (d, J=13.2 Hz, 4H), 1.76 (qd, J =7.2, 5.2 Hz, 1H), 0.78 (dd, J=6.2, 3.4 Hz, 4H); LC-MS: m/z 412.2 (MH+).

Preparation: N-[4-(3-phenylmorpholine-4-carbonyl)-3-pyrrolidin-1-ylphenyl]cyclopropanecarboxamide

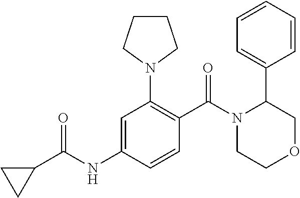 [SMILES: O=C(Nc1ccc(C(=O)N2CCOCC2c2ccccc2)c(N2CCCC2)c1)C1CC1]

### Example 167

The synthesis of the title compound was effected analogously to the synthesis of the compound of Example 166 (N-[3-pyrrolidin-1-yl-4-[2-(trifluoromethyl)morpholine-4-carbonyl]phenyl]cyclopropanecarboxamide) wherein 3-phenylmorpholine was used instead of 2-(trifluoromethyl)morpholine hydrochloride. The title compound was obtained in a yield of 98% (75 mg) as a racemic mixture.

1H NMR (500 MHz, DMSO-d6) δ ppm 10.30-9.97 (m, 1 H), 7.69-7.18 (m, 5 H), 7.17-6.72 (m, 3 H, 5.73-4.55 (m, 1 H), 4.52-3.40 (m, 4 H), 4.40-3.07 (m, 2 H), 3.40-2.65 (m, 4 H), 2.03-1.52 (m, 5 H), 0.89-0.67 (m, 4 H); LC-MS: m/z 420.47 (MH+).

Preparation: N-[4-(4-fluoropiperidine-1-carbonyl)-3-pyrrolidin-1-ylphenyl]cyclopropanecarboxamide

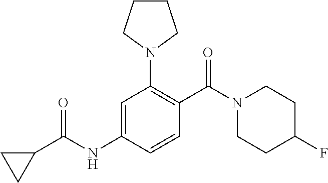 [SMILES: O=C(Nc1ccc(C(=O)N2CCC(F)CC2)c(N2CCCC2)c1)C1CC1]

### Example 168

The synthesis of the title compound was effected analogously to the synthesis of the compound of Example 166 (N-[3-pyrrolidin-1-yl-4-[2-(trifluoromethyl)morpholine carbonyl]phenyl]cyclopropanecarboxamide) wherein 4-fluoropiperidine;hydrochloride was used instead of 2-(trifluoromethyl)morpholine hydrochloride. The title compound was obtained in a yield of 35% (23 mg).

1H NMR (400 MHz, DMSO-d6) δ 10.09 (s, 1H), 7.06 (d, 1H), 6.93 (d, 2H), 5.00-4.78 (m, 1H), 3.91-3.66 (m, 2H), 3.51 (d, 1H), 3.23-2.90 (m, 5H), 2.00-1.44 (m, 9H), 0.78 (ddd, 4H); LC-MS: m/z 360.2 (MH+).

Preparation: N-[4-(1-oxo-3-phenyl-1,4-thiazinane-4-carbonyl)-3-pyrrolidin-1-ylphenyl]cyclopropanecarboxamide

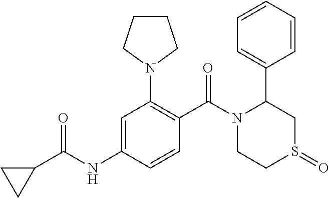 [SMILES: O=C(Nc1ccc(C(=O)N2CCS(=O)CC2c2ccccc2)c(N2CCCC2)c1)C1CC1]

### Example 169

To a solution of N-[4-(3-phenylthiomorpholine-4-carbonyl)-3-pyrrolidin-1-ylphenyl]cyclopropanecarboxamide (40.0 mg, 0.090 mmol) in DCM (6 mL), 3-chlorobenzenecarboperoxoic acid (33.28 mg, 0.190 mmol) was added and the reaction was allowed to stir at room temperature overnight. After this time the reaction was diluted with a saturated solution of NaHCO₃ and extracted with DCM. The organic phase was dried using a phase separator and concentrated to give a crude that was purified by FC on silica gel (eluting: from 100% of AcOEt to AcOEt/MeOH 90:10) to afford the product of formula N-[4-(1-oxo-3-phenyl-1,4-thiazinane-4-carbonyl)-3-pyrrolidin-1-ylphenyl]cyclopropanecarboxamide as racemic mixture.
Yield: 22.5 mg

1H NMR (500 MHz, DMSO-d6) δ ppm 10.35 -10.00 (m, 1 H), 7.50-7.21 (m, 5 H), 7.22-7.06 (m, 1 H), 7.26-b 6.71 (m, 2 H), 6.38-5.11 (m, 1 H), 5.07-3.71 (m, 2 H), 3.66-2.53 (m, 8 H), 2.08-1.58 (m, 5 H), 1.00-0.55 (m, 4 H); LC-MS: m/z 452.24

Preparation: N-[4-[2-(2-chlorophenyppiperazine-1-carbonyl]-3-pyrrolidin-1-ylphenyl]cyclopropanecarboxamide

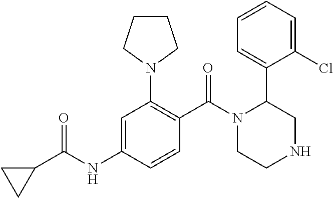 [SMILES: O=C(Nc1ccc(C(=O)N2CCNCC2c2ccccc2Cl)c(N2CCCC2)c1)C1CC1]

The synthesis of the title compound was effected analogously to the synthesis of the compound of Example 157 (N-[4-[2-(2-fluorophenyl)piperazine-1-carbonyl]-3-pyrrolidin-1-ylphenyl]cyclopropanecarboxamide) wherein tert-butyl 3-(2-chlorophenyl)-4-[4-(cyclopropanecarbonylamino)-2-pyrrolidin-1-ylbenzoyl]piperazine-1-carboxylate was used instead of tert-butyl 4[4-(cyclopropanecarbonylamino)-2-pyrrolidin-1-ylbenzoyI]-3-(2-fluorophenyl)piperazine-1-carboxylate. The title compound was obtained in a yield of 96% (35 mg) as a racemic mixture.
Lc-MS: m/z 453.39 (MH+)
   Preparation: N-[4-[2-(3-chlorophenyppiperazine-1-carbonyl]-3-pyrrolidin-1-ylphenyl]cyclopropanecarboxamide

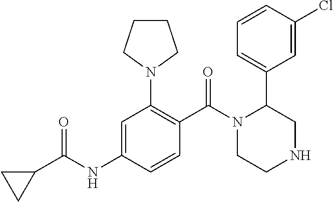 [SMILES: O=C(Nc1ccc(C(=O)N2CCNCC2c2cccc(Cl)c2)c(N2CCCC2)c1)C1CC1]

The synthesis of the title compound was effected analogously to the synthesis of the compound of Example 157 (N-[4-[2-(2-fluorophenyl)piperazine-1-carbonyl]-3-pyrrolidin-1-ylphenyl]cyclopropanecarboxamide) wherein tert-butyl 3-(3-chlorophenyl)-4-[4-(cyclopropanecarbonylamino) pyrrolidin-1-ylbenzoyl]piperazine-1-carboxylate was used instead of tert-butyl 4[4-(cyclopropanecarbonylamino)-2-pyrrolidin-1-ylbenzoyI]-3-(2-fluorophenyl)piperazine-1-carboxylate. The title compound was obtained in a yield of 84% (46 mg) as a racemic mixture.

Preparation: N-[4-[2-(5-chloropyridin-3-yppiperazine-1-carbonyl]-3-pyrrolidin-1-ylphenyl]cyclopropanecarboxamide

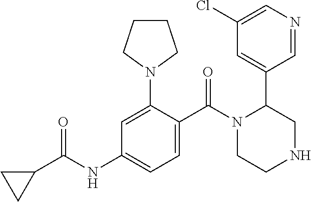 [SMILES: O=C(Nc1ccc(C(=O)N2CCNCC2c2cncc(Cl)c2)c(N2CCCC2)c1)C1CC1]

The synthesis of the title compound was effected analogously to the synthesis of the compound of Example 157 (N-[4-[2-(2-fluorophenyl)piperazine-1-carbonyl]-3-pyrrolidin-1-ylphenyl]cyclopropanecarboxamide) wherein tert-butyl 3-(5-chloropyridin-3-yl)-4-[4-(cyclopropanecarbonylamino)-2-pyrrolidin-1-ylbenzoyl]piperazine-1-carboxylate was used instead of tert-butyl 4[4-(cyclopropanecarbonylamino)-2-pyrrolidin-1-ylbenzoyl]-3-(2-fluorophenyl)piperazine-1-carboxylate. The title compound was obtained in a yield of 87% (39 mg) as a racemic mixture.
LC-MS: m/z 454.20 (MH+).
   Preparation: N-[4-(2-pyridin-3-ylpiperazine-1-carbonyl)-3-pyrrolidin-1-ylphenyl]cyclopropanecarboxamide

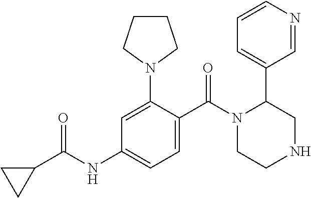 [SMILES: O=C(Nc1ccc(C(=O)N2CCNCC2c2cccnc2)c(N2CCCC2)c1)C1CC1]

The synthesis of the title compound was effected analogously to the synthesis of the compound of Example 157 (N-[4-[2-(2-fluorophenyl)piperazine-1-carbonyl]-3-pyrrolidin-1-ylphenyl]cyclopropanecarboxamide) wherein tert-butyl 4[4-(cyclopropanecarbonylamino)-2-pyrrolidin-1-ylbenzoyl]-3-pyridin-3-ylpiperazine-1-carboxylate was used instead of tert-butyl 4[4-(cyclopropanecarbonylamino)-2-pyrrolidin-1-ylbenzoyI]-3-(2-fluorophenyl)piperazine-1-carboxylate. The title compound was obtained in a yield of 91% (47 mg) as a racemic mixture.
LC-MS: m/z 420.22 (MH+).
   Preparation: N-[4-[2-(3-chloropyridin-4-yppiperazine-1-carbonyl]-3-pyrrolidin-1-ylphenyl]cyclopropanecarboxamide

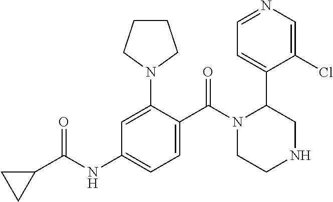 [SMILES: O=C(Nc1ccc(C(=O)N2CCNCC2c2ccncc2Cl)c(N2CCCC2)c1)C1CC1]

The synthesis of the title compound was effected analogously to the synthesis of the compound of Example 157 (N-[4-[2-(2-fluorophenyppiperazine-1-carbonyl]-3-pyrrolidin-1-ylphenyl]cyclopropanecarboxamide) wherein tert-butyl 3-(3-chloropyridin-4-yl)-4-[4-(cyclopropanecarbonylamino)-2-pyrrolidin-1-ylbenzoyl]piperazine-1-carboxylate was used instead of tert-butyl 4[4-(cyclopropanecarbonylamino)-2-pyrrolidin-1-ylbenzoyl]-3-(2-fluorophenyppiperazine-1-carboxylate. The title compound was obtained in a yield of 100% (20 mg) as a racemic mixture.
LC-MS: m/z 454.12 (MH+).
   Preparation: N-[4-(2-pyrimidin-5-ylpiperazine-1-carbonyl)-3-pyrrolidin-1-ylphenyl]cyclopropanecarboxamide

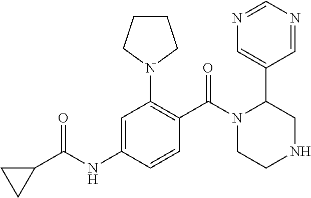 [SMILES: O=C(Nc1ccc(C(=O)N2CCNCC2c2cncnc2)c(N2CCCC2)c1)C1CC1]

The synthesis of the title compound was effected analogously to the synthesis of the compound of Example 157 (N-[4-[2-(2-fluorophenyppiperazine-1-carbonyl]-3-pyrrolidin-1-ylphenyl]cyclopropanecarboxamide) wherein tert-butyl 4[4-(cyclopropanecarbonylamino)-2-pyrrolidin-1-ylbenzoyl]-3-pyrimidin-5-ylpiperazine-1-carboxylate was used instead of tert-butyl 4[4-(cyclopropanecarbonylamino)-2-pyrrolidin-1-ylbenzoyl]-3-(2-fluorophenyl)piperazine-1-carboxylate. The title compound was obtained in a yield of 100% (45 mg) as a racemic mixture.
LC-MS: m/z 421.28 (MH+).
   Preparation: N-[4-[2-(4-chlorophenyppiperazine-1-carbonyl]-3-pyrrolidin-1-ylphenyl]cyclopropanecarboxamide

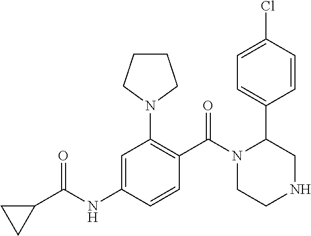 [SMILES: O=C(Nc1ccc(C(=O)N2CCNCC2c2ccc(Cl)cc2)c(N2CCCC2)c1)C1CC1]

The synthesis of the title compound was effected analogously to the synthesis of the compound of Example 157 N-[4-[2-(2-fluorophenyl)piperazine-1-carbonyl]-3-pyrrolidin-1-ylphenyl] cyclopropanecarboxamide wherein tert-butyl 3-(4-chlorophenyl)-4-[4-(cyclopropanecarbonylamino)-2-pyrrolidin-1-ylbenzoyl]piperazine-1-carboxylate was used instead of tert-butyl 4-[4-(cyclopropanecarbonylamino)-2-pyrrolidin-1-ylbenzoyI]-3-(2-fluorophenyl)piperazine-1-carboxylate. The title compound was obtained in a yield of 76% (33 mg) as a racemic mixture.
LC-MS: m/z 453.27 (MH+)
   Preparation: N-[4-[2-(2-chlorophenyl)-4-methylpiperazine-1-carbonyl]-3-pyrrolidin-1-ylphenyl]cyclopropanecarboxamide

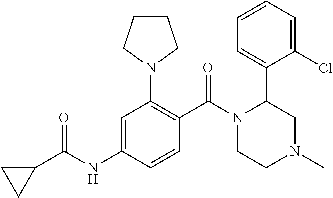 [SMILES: CN1CCN(C(=O)c2ccc(NC(=O)C3CC3)cc2N2CCCC2)C(c2ccccc2Cl)C1]

### Example 170a, 170b, 170c

The synthesis of the title compound was effected analogously to the synthesis of the compound of Example 88a-c N-[4-[2-(3-fluorophenyl)-4-methylpiperazine-1-carbonyl]-3-pyrrolidin-1-ylphenyl]cyclopropanecarboxamide wherein N-[4-[2-(2-chlorophenyl)piperazine-1-carbonyl]-3-pyrrolidin-1-yl phenyl]cyclopropanecarboxamide was used instead of N-[4-[2-(3-fluorophenyppiperazine-1-carbonyl]-3-pyrrolidin-1-yl phenyl]cyclopropanecarboxamide. The title compound was obtained in a yield of 78% (28 mg) as a racemic mixture.

1H NMR (500 MHz, DMSO-d6) δ ppm 10.20-9.92 (m, 1 H), 7.95-6.41 (m, 7 H), 5.98-5.04 (m, 1 H), 4.65-3.38 (m, 2 H), 3.28-3.08(m, 4 H), 2.20-2.09 (m, 3 H), 1.82-1.72 (m, 1 H), 3.10-1.66 (m, 4 H), 2.05-1.63 (m, 4 H), 0.78 (br s, 4 H); LC-MS: m/z 467.24 (MH+).

The racemic mixture (Example 170c) was then separated into single enantiomers by preparative HPLC

| | Column | Chiralpak IC (25 × 2.0 cm), 5μ | |
|---|---|---|---|
| | Mobile phase | n-Hexane/Ethanol 75/25% v/v | |
| | Flow rate (ml/min) | 17 ml/min | |
| | DAD detection | 220 nm | |
| Preparative chiral | Loop | 1750 μL | |
| chromatography | Total amount | 25 mg | |
| protocol: | Injection | 12.5 mg (each injection) | |
| 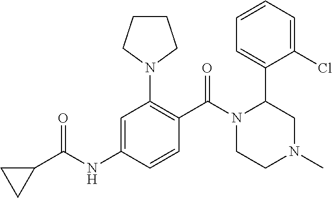 [SMILES: CN1CCN(C(=O)c2ccc(NC(=O)C3CC3)cc2N2CCCC2)C(c2ccccc2Cl)C1] | Enantiomer 1 | Rt 10.30 min | 100% ee |
| 2R or 2S enantiomer | LC-MS: m/z 467.24 (MH+ | | |
| | (2R or 2S) N-[4-[2-(2-chlorophenyl)-4-methylpiperazine-1- | | |
| | carbonyl]-3-pyrrolidin-1-ylphenyl] cyclopropanecarboxamide | | |
| | Example 170a | | |
| 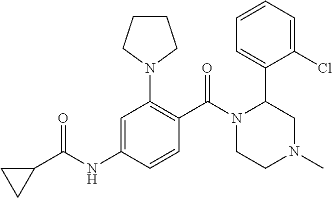 [SMILES: CN1CCN(C(=O)c2ccc(NC(=O)C3CC3)cc2N2CCCC2)C(c2ccccc2Cl)C1] | Enantiomer 2 | Rt 15.03 min | 100% ee |

| | |
|---|---|
| 2R or 2S enantiomer | LC-MS: m/z 467.24 (MH+) |
| | (2R or 2S) N-[4-[2-(2-chlorophenyl)-4-methylpiperazine-1- |
| | carbonyl]-3-pyrrolidin-1-ylphenyl]cyclopropanecarboxamide |
| | Example 170b |

Preparation: N-[4-[2-(3-chlorophenyl)-4-methylpiperazine-1-carbonyl]-3-pyrrolidin-1-ylphenyl]cyclopropanecarboxamide

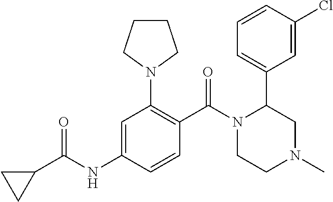 [SMILES: CN1CCN(C(=O)c2ccc(NC(=O)C3CC3)cc2N2CCCC2)C(c2cccc(Cl)c2)C1]

### Example 171a, 171b, 171c

The synthesis of the title compound was effected analogously to the synthesis of the compound of Example 88a-c N-[4-[2-(3-fluorophenyl)-4-methylpiperazine-1-carbonyl]-3-pyrrolidin-1-ylphenyl]cyclopropanecarboxamide wherein N-[4-[2-(3-chlorophenyl)piperazine-1-carbonyl]-3-pyrrolidin-1-ylphenyl]cyclopropanecarboxamide was used instead of N-[4-[2-(3-fluorophenyl)piperazine-1-carbonyl]-3-pyrrolidin-1-ylphenyl]cyclopropanecarboxamide. The title compound was obtained in a yield of 82% (39 mg) as a racemic mixture.

1H NMR (500 MHz, DMSO-d6) δ 10.25-10.00 (m, 1 H), 7.75-7.19 (m, 4 H), 7.18-7.02 (m, 1 H), 7.01-6.76 (m, 2 H), 5.88-4.64 (m, 1 H), 4.50-3.13 (m, 4 H), 2.29-2.17 (m, 3 H), 3.11-2.12 (m, 6 H), 1.81-1.72 (m, 1 H), 2.10-1.58 (m, 4 H), 0.83-0.71 (m, 4 H); LC-MS: m/z 467.28 (MH+)

The racemic mixture (Example 171c) was then separated into single enantiomers by preparative HPLC

| | | |
|---|---|---|
| | Column | Chiralpak AD-H (25 × 2.0 cm), 5μ |
| | Mobile phase | n-Hexane/Ethanol/MeOH 1/1 + |
| | | isopropylamine 55/45% v/v |
| | Flow rate (ml/min) | 17 ml/min |
| | DAD detection | 220 nm |
| Preparative chiral | Loop | 1300 μL |
| chromatography | Total amount | 34 mg |
| protocol: | Injection | 15 mg (each injection) |

| 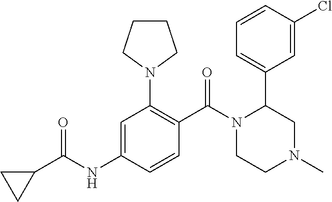 [SMILES: CN1CCN(C(=O)c2ccc(NC(=O)C3CC3)cc2N2CCCC2)C(c2cccc(Cl)c2)C1] | Enantiomer 1 | Rt 10.30 min | 100% ee |
|---|---|---|---|
| 2R or 2S enantiomer | LC-MS: m/z 467.28 (MH+) | | |
| | (2R or 2S) N-[4-[2-(3-chlorophenyl)-4-methylpiperazine-1- | | |
| | carbonyl]-3-pyrrolidin-1-ylphenyl] cyclopropanecarboxamide | | |
| | Example 171a | | |
| 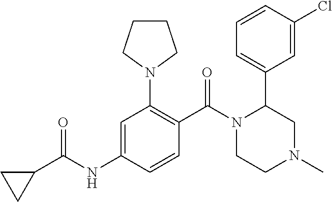 [SMILES: CN1CCN(C(=O)c2ccc(NC(=O)C3CC3)cc2N2CCCC2)C(c2cccc(Cl)c2)C1] | Enantiomer 2 | Rt 15.03 min | 100% ee |

| | |
|---|---|
| 2R or 2S enantiomer | LC-MS: m/z 467.28 (MH+) |
| | (2R or 2S) N-[4-[2-(3-chlorophenyl)-4-methylpiperazine-1- |
| | carbonyl]-3-pyrrolidin-1-ylphenyl] cyclopropanecarboxamide |
| | Example 171b |

Preparation: N-[4-[2-(5-chloropyridin-3-yl)-4-methylpiperazine-1-carbonyl]-3-pyrrolidin-1ylphenyl]cyclopropanecarboxamide

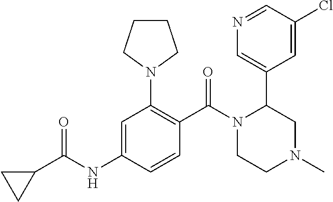 [SMILES: CN1CCN(C(=O)c2ccc(NC(=O)C3CC3)cc2N2CCCC2)C(c2cncc(Cl)c2)C1]

### Example 172

The synthesis of the title compound was effected analogously to the synthesis of the compound of Example 88a-c N-[4-[2-(3-fluorophenyl)-4-methylpiperazine-1-carbonyl]-3-pyrrolidin-1-ylphenyl]cyclopropanecarboxamide wherein N-[4-[2-(5-chloropyridin-3-yl)piperazine-1-carbonyl]-3-pyrrolidin-1-ylphenyl]cyclopropanecarboxamide was used instead of N-[4-[2-(3-fluorophenyl)piperazine-1-carbonyl]-3-pyrrolidin-1-ylphenyl]cyclopropanecarboxamide. The title compound was obtained in a yield of 63% (25 mg) as a racemic mixture. 1H NMR (400 MHz, DMSO-d6) δ ppm 10.18-10.09 (m, 1 H), 8.82 (d, J=1.76 Hz, 1 H), 8.64-8.47 (m, 1 H), 8.33-8.08 (m, 1 H), 7.98-7.16 (m, 1 H), 7.16 (br s, 1 H), 7.12-6.78 (m, 1 H), 5.84 (br d, J=2.64 Hz, 1 H), 5.73 (d, J=3.52 Hz, 1 H), 3.43 (br dd, J=11.33, 2.97 Hz, 1 H), 3.27-3.17 (m, 2 H), 3.09-2.82 (m, 2 H), 2.82-2.59 (m, 2 H), 2.39 (dd, J=11.99, 4.29 Hz, 1 H), 2.34-2.16 (m, 3 H), 2.10-1.94 (m, 1 H), 1.94-1.84(m, 2 H), 1.82-1.61 (m, 3 H), 0.84-0.71 (m, 4 H); LC-MS: m/z 468.30 (MH+).

Preparation: N-[4-(4-methyl-2-pyridin-3-ylpiperazine-1-carbonyl)-3-pyrrolidin-1-ylphenyl]cyclopropanecarboxamide

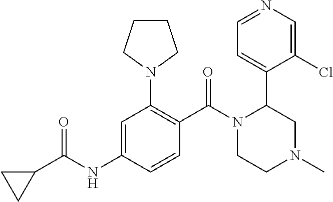 [SMILES: CN1CCN(C(=O)c2ccc(NC(=O)C3CC3)cc2N2CCCC2)C(c2ccncc2Cl)C1]

### Example 173

The synthesis of the title compound was effected analogously to the synthesis of the compound of Example 88a-c N-[4-[2-(3-fluorophenyl)-4-methylpiperazine-1-carbonyl]-3-pyrrolidin-1-ylphenyl]cyclopropanecarboxamide wherein N-[4-(2-pyridin-3-ylpiperazine-1-carbonyl)-3-pyrrolidin-1-ylphenyl]cyclopropanecarboxamide was used instead of N-[4-[2-(3-fluorophenyppiperazine-1-carbonyl]-3-pyrrolidin-1-ylphenyl]cyclopropanecarboxamide. The title compound was obtained in a yield of 88% (43 mg) as a racemic mixture.

1H NMR (400 MHz, DMSO-d6) δ 10.11 (d, 1H), 8.93-8.27 (m, 2H), 8.16-7.61 (m, 1H), 7.53-7.29 (m, 1H), 7.24-6.76 (m, 3H), 5.93-4.18 (m, 1H), 3.31 (s, 4H), 3.08-2.59 (m, 4H), 2.46-2.12 (m, 4H), 1.98 (d, 3H), 1.85-1.54 (m, 3H), 0.78 (dd, 4H); LC-MS: m/z 434.26 (MH+).

Preparation: N-[4-[2-(3-chloropyridin-4-yl)-4-methylpiperazine-1-carbonyl]-3-pyrrolidin-1-ylphenyl]cyclopropanecarboxamide

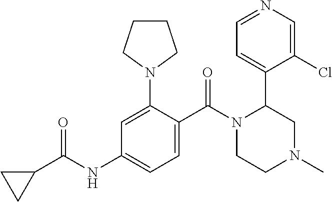 [SMILES: CN1CCN(C(=O)c2ccc(NC(=O)C3CC3)cc2N2CCCC2)C(c2ccncc2Cl)C1]

### Example 174

The synthesis of the title compound was effected analogously to the synthesis of the compound of Example 88a-c N-[4-[2-(3-fluorophenyl)-4-methylpiperazine-1-carbonyl]-3-pyrrolidin-1-ylphenyl]cyclopropanecarboxamide wherein N-[4-[2-(3-chloropyridin-4-yppiperazine-1-carbonyl]-3-pyrrolidin-1-ylphenyl]cyclopropanecarboxamide was used instead of N-[4-[2-(3-fluorophenyppiperazine-1-carbonyl]-3-pyrrolidin-1-ylphenyl]cyclopropanecarboxamide. The title compound was obtained in a yield of 73% (17 mg) as a racemic mixture.

1H NMR (400 MHz, DMSO-d6) δ 10.13 (s, 1H), 8.75-8.38 (m, 2H), 7.92-7.42 (m, 1H), 7.20-6.84 (m, 3H), 6.19-4.31 (m, 1H), 3.74-3.45 (m, 2H), 3.29-3.00 (m, 4H), 3.00-2.62 (m, 2H), 2.47-2.30 (m, 1H), 2.21- 2.09 (m, 3H), 2.02-1.64 (m, 6H), 0.93-0.53 (m, 4H); LC-MS: m/z 468.25 (MH+).

Preparation: N-[4-(4-methyl-2-pyrimidin-5-ylpiperazine-1-carbonyl)-3-pyrrolidin ylphenyl]cyclopropanecarboxamide

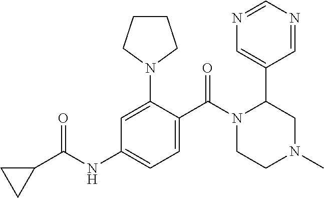 [SMILES: CN1CCN(C(=O)c2ccc(NC(=O)C3CC3)cc2N2CCCC2)C(c2cncnc2)C1]

### Example 175

The synthesis of the title compound was effected analogously to the synthesis of the compound of Example 88a-c N-[4-[2-(3-fluorophenyl)-4-methylpiperazine-1-carbonyl]-3-pyrrolidin-1-ylphenyl]cyclopropanecarboxamide wherein N-[4-(2-pyrimidin-5-ylpiperazine-1-carbonyl)-3-pyrrolidin-1-ylphenyl]cyclopropanecarboxamide was used instead of N-[4-[2-(3-fluorophenyppiperazine-1-carbonyl]-3-pyrrolidin-1-ylphenyl]cyclopropanecarboxamide. The title compound was obtained in a yield of 55% (26 mg) as a racemic mixture.

1H NMR (400 MHz, DMSO-d6) δ 10.13 (d, 1H), 9.10 (d, 2H), 8.91-8.53 (m, 1H), 7.30-6.76 (m, 3H), 5.96-4.28 (m, 1H), 3.44 (d, 1H), 3.23 (s, 2H), 3.13-2.93 (m, 2H), 2.92-2.62 (m, 2H), 2.45-2.16 (m, 4H), 2.11-1.84 (m, 4H), 1.82-1.60 (m, 3H), 0.78 (td, 4H); LC-MS: m/z 435.21 (MH+).

Preparation: N-[4-[2-(4-chlorophenyl)-4-methylpiperazine-1-carbonyl]-3-pyrrolidin-1-ylphenyl]cyclopropanecarboxamide

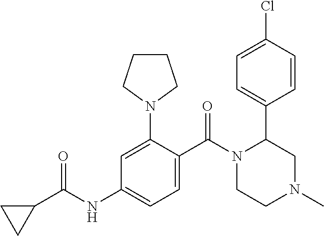 [SMILES: CN1CCN(C(=O)c2ccc(NC(=O)C3CC3)cc2N2CCCC2)C(c2ccc(Cl)cc2)C1]

### Example 176

The synthesis of the title compound was effected analogously to the synthesis of the compound of Example 88a-c N-[4-[2-(3-fluorophenyl)-4-methylpiperazine-1-carbonyl]-3-pyrrolidin-1-ylphenyl]cyclopropanecarboxamide wherein N-[4-[2-(4-chlorophenyl)piperazine-1-carbonyl]-3-pyrrolidin-1-ylphenyl]cyclopropanecarboxamide was used instead of N-[4-[2-(3-fluorophenyl)piperazine-1-carbonyl]-3-pyrrolidin-1-ylphenyl]cyclopropanecarboxamide. The title compound was obtained in a yield of 88% (30 mg) as a racemic mixture.

1H NMR (500 MHz, DMSO-d6) δ 10.26-9.99 (m, 1 H), 7.79-7.17 (m, 4 H), 7.17-7.00 (m, 1 H), 7.02-6.75(m, 2 H), 5.88-4.61 (m, 1 H), 3.47-3.10 (m, 4 H), 2.27-2.15 (m, 3 H), 3.27-2.11 (m, 6 H), 1.79-1.73 (m, 1 H), 2.04-1.58 (m, 4 H), 0.83-0.71 (m, 4 H); LC-MS: m/z 467.25 (MH+).

Preparation: 4-(4-methyl-2-phenylpiperazine-1-carbonyl)-3-[3-(trifluoromethyl)pyrrolidin-1-yl]benzonitrile

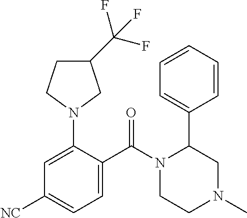 [SMILES: CN1CCN(C(=O)c2ccc(C#N)cc2N2CCC(C(F)(F)F)C2)C(c2ccccc2)C1]

The synthesis of the title compound was effected analogously to the synthesis of 4-(4-methyl-2-phenylpiperazine-1-carbonyl)-3-pyrrolidin-1-ylbenzonitrile wherein 3-(trifluoromethyl)pyrrolidine hydrochloride was used instead of 3pyrrolidine. The title compound was obtained in a yield of 60% (163mg) as mixture of diasteroisomers.
LC-MS: m/z 443.23 (MH+).
   Preparation: (4-methyl-2-phenylpiperazin-1-yl)-[4-(5-methyl-4H-1,2,4-triazol-3-yl)-2-[3-(trifluoromethyl)pyrrolidin-1-yl]phenyl]methanone

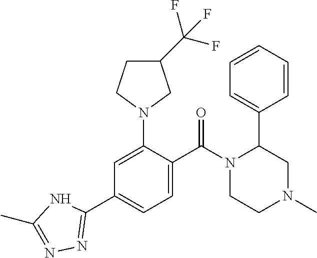

### Example 177a, 177b, 177c, 177d

A mixture of dicesium carbonate (360.07 mg, 1.11 mmol), bromocopper (2.64 mg, 0.020 mmol), 4-(4-methyl-2-phenylpiperazine-1-carbonyl)-3-[3-(trifluoromethyl)pyrrolidin-1-yl]benzonitrile (163.0 mg, 0.370 mmol) and ethanimidamide hydrochloride (52.24 mg, 0.550 mmol) in DMSO (2.709 mL) was heated at 110° C. for 24 h. After this time the reaction was cooled down to RT and purified by FC on NH column (eluting from cHex/AcOEt 80:20 to cHex/AcOEt 40:60) affording the product of formula (4-methyl-2-phenylpiperazin-1-yl)-[4-(5-methyl-4H-1,2,4-triazol-3-yl)-2-[3-(trifluoromethyl)pyrrolidin-1-yl]phenyl]methanone as mixture of diasteroisomers in a yield of 76% (140 mg).
LC-MS: m/z 499.2 (MH+)
   The mixture of diasteroisomers was then separated into single enantiomers by chiral semi-preparative HPLC

| | | |
|---|---|---|
| | Column | Whelk O1 (R, R) (25 × 3.0 cm), 10μ |
| | Mobile phase | n-Hexane/Ethanol + isopropylamine |
| | | 75/25% v/v |
| | Flow rate (ml/min) | 37 ml/min |
| | DAD detection | 220 nm |
| Preparative chiral | Loop | 700 μL |
| chromatography | Total amount | 139 mg |
| protocol: | Injection | 9.7 mg (each injection) |

Diasteroisomer 1

| 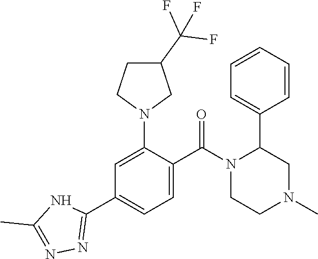 | Enantiomer 1, Yield: 18 mg | Rt 12.014 min | 100% ee |
|---|---|---|---|
| (2S, 3R) or (2R, 3S) or (2R, 3R) or (2S, 3S) | LC-MS: m/z 499.27 (MH+) | | |
| | (2S, 3R) or (2R, 3S) or (2R, 3R) or (2S, 3S) (4-methyl-2- | | |
| | phenylpiperazin-1-yl)-[4-(5-methyl-4H-1,2,4-triazol-3-yl)-2- | | |
| | [3-(trifluoromethyl)pyrrolidin-1-yl]phenyl]methanone | | |
| | Example 177a | | |
| 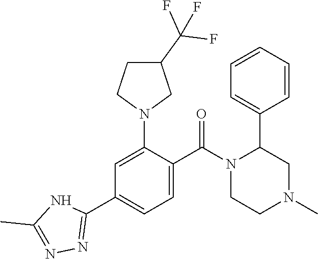 | Enantiomer 2, Yield: 18 mg | Rt 33.575 min | 100% ee |

| | |
|---|---|
| (2S, 3R) or (2R, 3S) or (2R, 3R) or (2S, 3S) | LC-MS: m/z 499.27 (MH+) |
| | (2S, 3R) or (2R, 3S) or (2R, 3R) or (2S, 3S) (4-methyl-2-phenyl- |
| | piperazin-1-yl)-[4-(5-methyl-4H-1,2,4-triazol-3-yl)-2-[3-(trifluoro- |
| | methyl)pyrrolidin-1-yl]phenyl]methanone |
| | Example 177b |

1H NMR (400 MHz, DMSO-d6) δ 13.73 (bs, 1H) 7.71 (d, 1H), 7.50 (d, 1H), 7.47-7.27 (m, 4H), 7.20 (d, 1H), 7.06 (dd,
1H), 5.96-4.35 (m, 1H), 3.76-3.37 (m, 4H), 3.23-2.59 (m, 4H), 2.46-2.23 (m, 6H), 2.23-1.81 (m, 5H).
Diasteroisomer 2

| | | | |
|---|---|---|---|
| 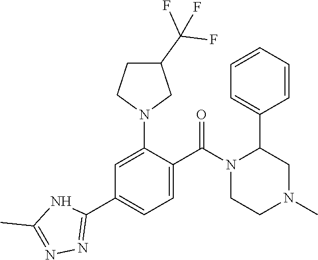 | Enantiomer 1, Yield: 17 mg | Rt 24.97 min | 100% ee |

| (2S, 3R) or (2R, 3S) or (2R, 3R) or (2S, 3S) | LC-MS: m/z 499.23 (MH+) | | |
|---|---|---|---|
| | (2S, 3R) or (2R, 3S) or (2R, 3R) or (2S, 3S) (4-methyl-2-phenyl- | | |
| | piperazin-1-yl)-[4-(5-methyl-4H-1,2,4-triazol-3-yl)-2-[3-(trifluoro- | | |
| | methyl)pyrrolidin-1-yl]phenyl]methanone | | |
| | Example 177c | | |
| 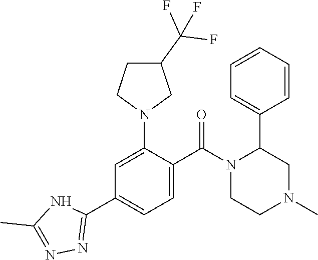 | Enantiomer 2, Yield: 15 mg | Rt 29.59 min | 100% ee |

| | |
|---|---|
| (2S, 3R) or (2R, 3S) or (2R, 3R) or (2S, 3S) | LC-MS: m/z 499.23 (MH+) |
| | (2S, 3R) or (2R, 3S) or (2R, 3R) or (2S, 3S) (4-methyl-2- |
| | phenylpiperazin-1-yl)-[4-(5-methyl-4H-1,2,4-triazol-3-yl)- |
| | 2-[3-(trifluoromethyl)pyrrolidin-1-yl]phenyl]methanone |
| | Example 177d |

1H NMR (400 MHz, DMSO-d6) δ 13.73 (s, 1H), (m, 2H), 7.46-6.95 (m, 6H), 5.88-4.37 (m, 1H), 3.30 (s,
6H), 3.22-2.95 (m, 2H), 2.83-2.60 (m, 2H), 2.44-2.23 (m, 7H), 2.15-1.58 (m, 2H).

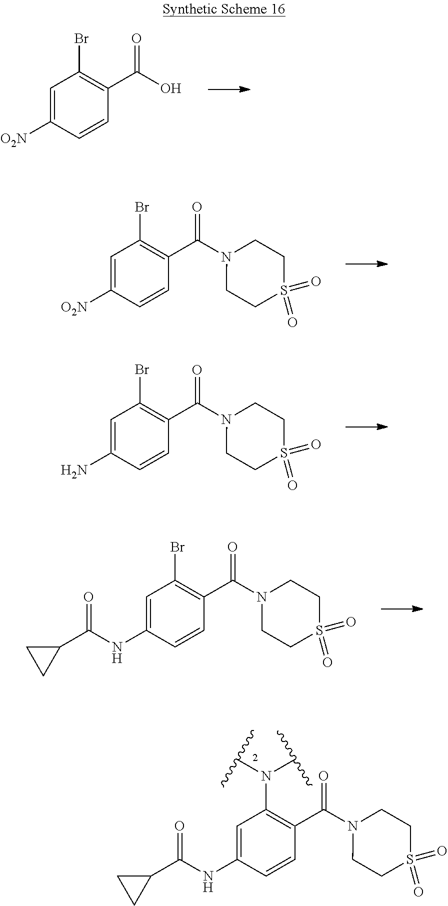 [SMILES: O=C(O)c1ccc([N+](=O)[O-])cc1Br ; O=C(c1ccc([N+](=O)[O-])cc1Br)N1CCS(=O)(=O)CC1 ; Nc1ccc(C(=O)N2CCS(=O)(=O)CC2)c(Br)c1 ; O=C(Nc1ccc(C(=O)N2CCS(=O)(=O)CC2)c(Br)c1)C1CC1 ; *N(*)c1cc(NC(=O)C2CC2)ccc1C(=O)N1CCS(=O)(=O)CC1]

Preparation: (2-bromo-4-nitrophenyI)-(1,1-dioxo-1,4-thiazinan-4-yl)methanone

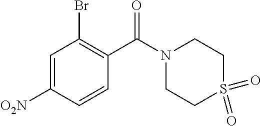 [SMILES: O=C(c1ccc([N+](=O)[O-])cc1Br)N1CCS(=O)(=O)CC1]

A mixture of 2-bromo-4-nitrobenzoic acid (3.0 g, 12.19 mmol) and thionyl dichloride (17.79 mL, 243.89 mmol) was heated to reflux for 1 h. After this time the reaction was cooled down to RT and concentrated under vacuum. The residue was dissolved in DCM (10.16 mL) and cooled to 0° C. After 10 min a solution of 1,4-thiazinane 1,1-dioxide hydrochloride (2.09 g, 12.19 mmol) and triethylamine (6.8 mL, 48.78 mmol) in DCM (50.81 mL) was added. The reaction was stirred at RT for 1h. After this time the reaction was diluted with DCM and washed with a s.s. of NaHCO₃ 3 times. The organic phase was dried over a phase separator and concentrated under vacuum affording the product of formula (2-bromo-4-nitrophenyI)-(1,1-dioxo-1,4-thiazinan-4-yl)methanone. The product was used in the next step without further purification.
Yield: 3.3 g

1H NMR (400 MHz, DMSO) δ 8.52 (d, J=2.2 Hz, 1H), 8.36 (dd, J=8.4, 2.2 Hz, 1H), 7.89 (d, J=8.4 Hz, 1H), 4.50-4.36 (m, 1H), 3.74 (ddd, J=13.6, 9.8, 2.9 Hz, 1H), 3.60-3.51 (m, 2H), 3.42 (d, J=14.6 Hz, 1H), 3.28-3.23 (m, 1H), 3.07 (s, 2H); LC-MS: m/z 364.94 (MH+)

Preparation: (4-amino-2-bromophenyl)-(1,1-dioxo-1,4-thiazinan-4-yl)methanone

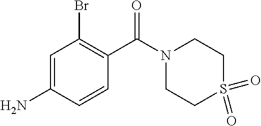 [SMILES: Nc1ccc(C(=O)N2CCS(=O)(=O)CC2)c(Br)c1]

(2-bromo-4-nitrophenyl)-(1,1-dioxo-1,4-thiazinan-4-yl)methanone (3.9 g, 10.74 mmol) and tin(III) chloride dehydrate (4.89 g, 21.48 mmol) were dissolved in Ethanol (53.69 mL) and the mixture was heated to reflux for 8 h. After this time the reaction was cooled down to RT and evaporated under vacuum. The residue was purified by FC on NH column (eluting from 100% of DCM to DCM/MeOH 95:5) to obtain the product of formula (4-amino-2-bromophenyl)-(1,1-dioxo-1,4-thiazinan-4-yl)methanone.
Yield: 2.96 g

1H NMR (400 MHz, DMSO-d6) δ 7.10 (d, J=8.3 Hz, 1H), 6.80 (d, J=2.1 Hz, 1H), 6.57 (dd, J=8.3, 2.1 Hz, 1H), 5.75 (s, 2H), 4.27 (s, 1H), 3.40 (d, J=44.2 Hz, 7H); LC-MS: m/z 333.0 (MH+)

Preparation: N-[3-bromo-4-(1,1-dioxo-1,4-thiazinane-4-carbonyl)phenyl]cyclopropanecarboxamide

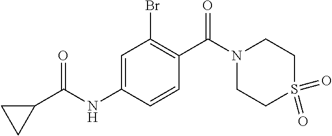 [SMILES: O=C(Nc1ccc(C(=O)N2CCS(=O)(=O)CC2)c(Br)c1)C1CC1]

To a mixture of (4-amino-2-bromophenyl)-(1,1-dioxo-1,4-thiazinan-4-yl)methanone (3.0 g, 9 mmol) and N,N-Diisopropylethylamine (2.3 mL, 13.51 mmol) in DCM (136.42 mL) at 0° C., cyclopropanecarbonyl chloride (0.74 mL, 8.1 mmol) was added dropwise and allowed to reach RT. After 1 h the reaction was concentrated under vacuum and a s. s. of NaHCO₃ was added. The aqueous phase was extracted three times with AcOEt. The combined organic fractions were washed with Brine, dried over Na₂SO₄, filtered and the solvent removed under vacuum to give the product of formula N-[3-bromo-4-(1,1-dioxo-1,4-thiazinane-4-carbonyl)phenyl]cyclopropanecarboxamide. The product was used in the next step without further purification.
Yield: 1.6 g

1H NMR (400 MHz, DMSO) δ 10.49 (s, 1H), 8.04 (d, J=2.0 Hz, 1H), 7.59 (dd, J=8.4, 2.0 Hz, 1H), 7.46 (d, J=8.4 Hz, 1H), 4.35 (s, 1H), 3.75 (s, 1H), 3.56 (s, 3H), 3.23 (s, 2H), 3.06 (s, 1H), 1.78 (p, J=6.3 Hz, 1H), 0.84 (d, J=7.4 Hz, 4H); LC-MS: m/z 401.27, 403.27 (MH+).

Preparation: N-[4-(1,1-dioxo-1,4-thiazinane-4-carbonyl)-3-[5-methyl-3-(trifluoromethyl)pyrazol-1-yl]phenyl]cyclopropanecarboxamide

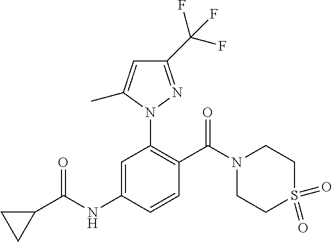 [SMILES: Cc1cc(C(F)(F)F)nn1-c1cc(NC(=O)C2CC2)ccc1C(=O)N1CCS(=O)(=O)CC1]

### Example 178

In a vial were sequentially added N-[3-bromo-4-(1,1-dioxo-1,4-thiazinane-4-carbonyl)phenyl]cyclopropanecarboxamide (150.0 mg, 0.370 mmol), tripotassium phosphate (169.0 mg, 0.780 mmol), 3-methyl-5-(trifluoromethyl)-1H-pyrazole (67.33 mg, 0.450 mmol) and suspended in a dry 1,4-Dioxane (1.869 mL). The resulting suspension was degassed bubbling N₂ for 15 minutes, then copper (I) iodide (3.58 mg, 0.020 mmol) and N,N′-Dimethylcyclohexane-1,2-diamine (5.89 uL, 0.040 mmol) were added under nitrogen flux and degassed again with Schlenk line technique. The vial was sealed and stirred for 48h at 100° C. After this time the reaction was cooled down to RT, diluted with

EtOAc and filtered through a pad of celite. The organic layer was washed with Brine, dried over Na₂SO₄, filtered and concentrated under vacuum. The residue was purified by FC on RP using acidic conditions (eluting from CH₃CN/H₂O 5:95 +0.1% of formic acid to CH₃CN/H₂O 50:50 +0.1% of formic acid) affording the product of formula N-[4-(1,1-dioxo-1,4-thiazinane-4-carbonyl)-3-[5-methyl-3-(trifluoromethyl)pyrazol-1-yl]phenyl]cyclopropanecarboxamide.
Yield:19.5 mg 1H NMR (500 MHz, DMSO-d6) δ ppm 10.67 (s, 1 H), 7.90 (d, J=2.1 Hz, 1 H), 7.70 (dd, J=8.4, 2.0 Hz, 1 H), 7.55 (d, J=8.4 Hz, 1 H), 6.74 (s, 1 H), 4.08-3.60 (m, 4 H), 3.26-2.70 (m, 4 H), 2.26 (s, 3 H), 1.91-1.70 (m, 1 H), 0.93-0.75 (m, 4 H); LC-MS: m/z471.39 (MH+).
   Preparation: N-[4-(1,1-dioxo-1,4-thiazinane-4-carbonyl)-3-(3-propan-2-ylpyrazol-1-yl)phenyl]cyclopropanecarboxamide

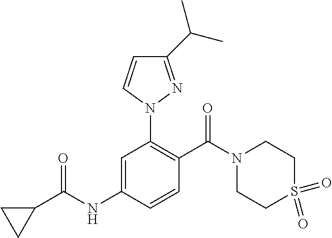 [SMILES: CC(C)c1ccn(-c2cc(NC(=O)C3CC3)ccc2C(=O)N2CCS(=O)(=O)CC2)n1]

### Example 179

The synthesis of the title compound was effected analogously to the synthesis of the compound of Example 178 (N-[4-(1,1-dioxo-1,4-thiazinane-4-carbonyl)-3[5-methyl-3-(trifluoromethyl)pyrazol-1-yl]phenyl]cyclopropanecarboxamide) wherein 3-propan-2-yl-1H-pyrazole was used instead of 3-methyl-5-(trifluoromethyl)-1H-pyrazole. The title compound was obtained in a yield of 6.5% (7 mg).

1H NMR (400 MHz, DMSO-d6) δ 10.54 (s, 1H), 7.91 (dd, J=6.4, 2.2 Hz, 2H), 7.57 (dd, J=8.4, 2.0 Hz, 1H), 7.40 (d, J=8.4 Hz, 1H), 6.39 (d, J=2.5 Hz, 1H), 4.02 (s, 1H), 3.86 (t, J=11.0 Hz, 1H), 3.66-3.43 (m, 2H), 3.30 (s, 1 H), 3.10 (d, J=13.4 Hz, 1H), 2.93 (dt, J=13.8, 6.9 Hz, 2H), 2.83 (d, J=10.5 Hz, 1H), 1.80 (p, J =6.2 Hz, 1H), 1.21 (d, J=6.9 Hz, 6H), 0.83 (d, J=6.3 Hz, 4H); LC-MS: 431.3 (MH+)

Preparation: N-[3-(3-cyclopropylpyrazol-1-yl)-4-(1,1-dioxo-1,4-thiazinane-4-carbonyl)phenyl]cyclopropanecarboxamide

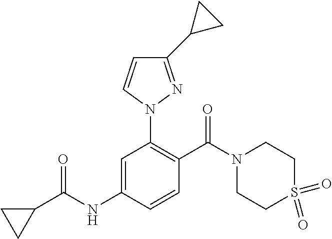 [SMILES: O=C(Nc1ccc(C(=O)N2CCS(=O)(=O)CC2)c(-n2ccc(C3CC3)n2)c1)C1CC1]

### Example 180

The synthesis of the title compound was effected analogously to the synthesis of the compound of Example 178 (N-[4-(1,1-dioxo-1,4-thiazinane-4-carbonyl)-3-[5-methyl-3-(trifluoromethyl)pyrazol-1-yl]phenyl]cyclopropanecarboxamide) wherein 3-cyclopropyl-1H-pyrazole was used instead of 3-methyl-5-(trifluoromethyl)-1H-pyrazole. The title compound was obtained in a yield of 13% (14 mg).

1H NMR (500 MHz, DMSO-d6) δ ppm 10.55 (s, 1 H), 7.92 (d, J=2.5 Hz, 1 H), 7.88 (d, J=1.8 Hz, 1 H), 7.55 (dd, J=8.4, 1.9 Hz, 1 H), 7.39 (d, J=8.4 Hz, 1 H), 6.21 (d, J=2.5 Hz, 1 H), 3.51-4.23 (m, 4 H), 2.94-3.31 (m, 4 H), 1.87-1.98 (m, 1 H), 1.80 (quin, J=6.2 Hz, 1 H), 0.88-0.96 (m, 2 H), 0.77-0.87 (m, 4 H), 0.60-0.75 (m, 2 H); m/z 429.2 (MH+).

Preparation: N-[4-(1,1-dioxo-1,4-thiazinane-4-carbonyl)-3-[3-(trifluoromethyl)pyrazol-1-yl]phenyl]cyclopropanecarboxamide

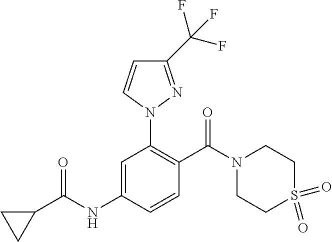 [SMILES: O=C(Nc1ccc(C(=O)N2CCS(=O)(=O)CC2)c(-n2ccc(C(F)(F)F)n2)c1)C1CC1]

### Example 181

The synthesis of the title compound was effected analogously to the synthesis of the compound of Example 178 (N-[4-(1,1-dioxo-1,4-thiazinane-4-carbonyl)-3-[5-methyl-3-(trifluoromethyl)pyrazol-1-yl]phenyl]cyclopropanecarboxamide) wherein 3-(trifluoromethyl)-1H-pyrazole was used instead of 3-methyl-5-(trifluoromethyl)-1H-pyrazole. The title compound was obtained in a yield of 18% (42 mg).

1H NMR (400 MHz, DMSO-d6) δ 10.65 (s, 1H), 8.37 (dd, J=2.5, 1.0 Hz, 1H), 8.02 (d, J=2.0 Hz, 1H), 7.67 (dd, J=8.4, 2.0 Hz, 1H), 7.51 (d, J=8.4 Hz, 1H), 7.01 (d, J=2.5 Hz, 1H), 4.01 (s, 1H), 3.84 (s, 1H), 3.68 (t, J =5.5 Hz, 2H), 3.25-2.85(m, 4H), 1.81 (p, J=6.3 Hz, 1H), 0.88-0.82 (m, 4H); LC-MS: m/z 457.4 (MH+).

Preparation: N-[4-(1,1-dioxo-1,4-thiazinane-4-carbonyl)-3-[4-(trifluoromethyl)pyrazol yl]phenyl]cyclopropanecarboxamide

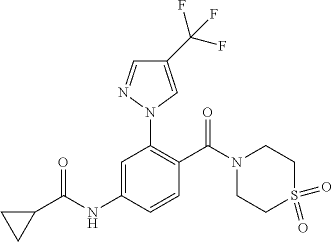 [SMILES: O=C(Nc1ccc(C(=O)N2CCS(=O)(=O)CC2)c(-n2cc(C(F)(F)F)cn2)c1)C1CC1]

### Example 182

The synthesis of the title compound was effected analogously to the synthesis of the compound of Example 178 (N-[4-(1,1-dioxo-1,4-thiazinane-4-carbonyl)-3-[5-methyl-3-(trifluoromethyl)pyrazol-1-yl]phenyl]cyclopropanecarboxamide) wherein 4-(trifluoromethyl)-1H-pyrazole was used instead of 3-methyl-5-(trifluoromethyl)-1H-pyrazole. The title compound was obtained in a yield of 5% (11 mg).

1H NMR (400 MHz, DMSO-d6) δ 10.63 (s, 1H), 8.83 (s, 1H), 8.18 (s, 1H), 8.02 (d, 1H), 7.65-7.60 (m, 1H), 7.52 (d, 1H), 3.92 (s, 2H), 3.62 (s, 2H), 3.11 (d, 4H), 1.81 (p, 1H), 0.84 (dd, 4H); LC-MS: m/z 457.10 (MH+)

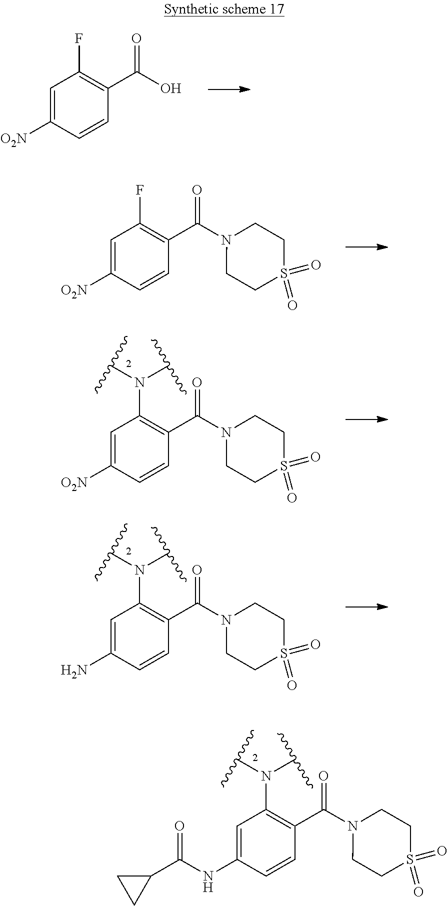 [SMILES: O=C(O)c1ccc([N+](=O)[O-])cc1F ; O=C(c1ccc([N+](=O)[O-])cc1F)N1CCS(=O)(=O)CC1 ; *N(*)c1cc([N+](=O)[O-])ccc1C(=O)N1CCS(=O)(=O)CC1 ; *N(*)c1cc(N)ccc1C(=O)N1CCS(=O)(=O)CC1 ; *N(*)c1cc(NC(=O)C2CC2)ccc1C(=O)N1CCS(=O)(=O)CC1]

Preparation: (1,1-dioxo-1,4-thiazinan-4-yl)-(2-fluoro-3-methyl-4-nitrophenyl)methanone

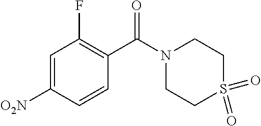 [SMILES: O=C(c1ccc([N+](=O)[O-])cc1F)N1CCS(=O)(=O)CC1]

2-fluoro-3-methyl-4-nitrobenzoic acid (145.0 mg, 0.730 mmol) and thionyl dichloride (1.06 mL, 14.56 mmol) were stirred at 80° C. for 2 h. After this time the reaction was cooled down to RT and concentrated under vacuum. The mixture was stripped three times with cyclohexane in order to remove thionyl dichloride in excess. The residue was dissolved with dry DCM (20 mL) and cooled to 0° C. Afer 10 min. a solution of 1,4-thiazinane 1,1-dioxide hydrochloride (124.98 mg, 0.730 mmol) and triethylamine (0.3 mL, 2.18 mmol) in dry DCM (10 mL) was added. The reaction mixture was allowed to reach the RT and stirred overnight. The day after the reaction was diluted with DCM and washed 3 times with a s.s. of NaHCO3. The organic phases were collected, washed with Brine, dried over a phase separator and concentrated under vacuum affording the product of formula (1,1-dioxo-1,4-thiazinan-4-yl)-(2-fluoro-3-methyl-4-nitrophenyl)methanone. The product was used in the next step without further purification.
Yield: 193 mg.

1H NMR (400 MHz, DMSO) δ 7.95 (dd, J=8.4, 1.2 Hz, 1H), 7.80-7.54 (m, 1H), 4.34 (d, J=39.6 Hz, 2H), 3.65 (s, 4H), 2.98 (d, J=65.2 Hz, 2H), 2.41 (d, J=2.2 Hz, 3H); LC-MS: 317.09 (MH+).

Preparation: (1,1-dioxo-1,4-thiazinan-4-yl)-[4-nitro-2-[3-(trifluoromethyl)pyrrolidin-1-yl]phenyl]methanone

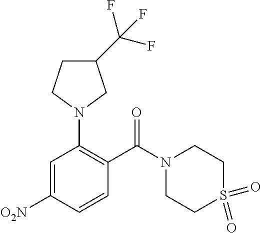 [SMILES: O=C(c1ccc([N+](=O)[O-])cc1N1CCC(C(F)(F)F)C1)N1CCS(=O)(=O)CC1]

To a solution of (1,1-dioxo-1,4-thiazinan-4-yl)-(2-fluoro-4-nitrophenyl)methanone (216.0 mg, 0.710 mmol) and 3-(trifluoromethyl)pyrrolidine hydrochloride (250.93 mg, 1.43 mmol) in DMSO (3.15 mL), N,N-Diisopropylethylamine (0.5 mL, 2.86 mmol) was added. The reaction was stirred at 120° C. for 48 h. After this time the reaction was cooled down to RT and diluted with water. The mixture was then extracted with AcOEt 3 times. The combined organic fractions were washed with Brine, dried over Na₂SO₄, filtered and concentrated under vacuum. The residue was purified by FC on silica gel (eluting from cHex/AcOEt 90:10 to 50:50) to give product of formula (1,1-dioxo-1,4-thiazinan-4-yl)-[4-nitro-2[3-(trifluoromethyl)pyrrolidin-1-yl]phenyl]methanone.
Yield: 209 mg

1H NMR (400 MHz, DMSO-d6) δ 7.50-7.62 (m, 2H), 7.47 (dd, J=6.8, 1.9 Hz, 1H), 4.48 (t, J=7.3 Hz, 1H), 3.78-3.49 (m, 4H), 3.44-3.31(m, 5H), 3.30-3.15 (m, 2H), 3.12-2.97 (m, 1H), 2.25 (dt, J=12.8, 6.7 Hz, 1H), 2.07 (dt, J=11.7, 7.6 Hz, 1H); LC-MS: 422.1 (MH+).

Preparation: [2-(3,3-difluoropyrrolidin-1-yl)-4-nitrophenyI]-(1,1-dioxo-1,4-thiazinan-4-yl)methanone

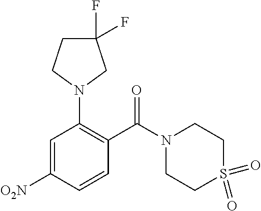 [SMILES: O=C(c1ccc([N+](=O)[O-])cc1N1CCC(F)(F)C1)N1CCS(=O)(=O)CC1]

The synthesis of the title compound was effected analogously to the synthesis of (1,1-dioxo-1,4-thiazinan-4-yl)-[4-nitro-2[3-(trifluoromethyl)pyrrolidin-1-yl]phenyl]methanone wherein 3,3-difluoropyrrolidine;hydrochloride was used instead of 3-(trifluoromethyl)pyrrolidine hydrochloride. The title compound was obtained in a yield of 32% (62 mg).

1H NMR (400 MHz, DMSO-d6) δ 7.61 (d, J=1.2 Hz, 2H), 7.48 (d, J=1.3 Hz, 1H), 4.48 (d, J=14.1 Hz, 1H), 3.80-3.59 (m, 5H), 3.52 (dtd, J=16.5, 9.6, 7.2 Hz, 2H), 3.45-3.36 (m, 1H), 3.30-3.21 (m, 2H), 3.12-3.02 (m, 1H), 2.46 (d, J=7.3 Hz, 2H); LC-MS: 390.0 (MH+).

Preparation: (1,1-dioxo-1,4-thiazinan-4-yl)-[4-nitro-2-[3-(trifluoromethyl)piperidin-1-yl]phenyl]methanone

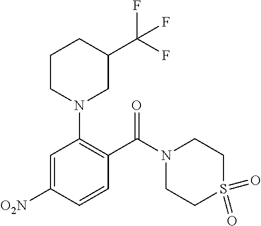 [SMILES: O=C(c1ccc([N+](=O)[O-])cc1N1CCCC(C(F)(F)F)C1)N1CCS(=O)(=O)CC1]

The synthesis of the title compound was effected analogously to the synthesis of (1,1-dioxo-1,4-thiazinan-4-yl)-[4-nitro-2-[3-(trifluoromethyl)pyrrolidin-1-yl ]phenyl]methanone wherein 3-(trifluoromethyl)piperidine hydrochloride was used instead of 3-(trifluoromethyl)pyrrolidine hydrochloride. The title compound was obtained in a yield of 60% (254 mg).

1H NMR (400 MHz, Chloroform-d) δ 8.00 (dd, J=8.3, 2.1 Hz, 1H), 7.97-7.86 (m, 1H), 7.49-7.38 (m, 1H), 4.60-4.45 (m, 1H), 4.15-3.99 (m, 1H), 3.66 (q, J=5.8 Hz, 3H), 3.44 (dd, J=66.7, 11.8 Hz, 1H), 3.18 (dq, J =10.2, 5.4 Hz, 3H), 3.08-2.96 (m, 2H), 2.87 (ddt, J=19.9, 12.6, 6.3 Hz, 1H), 2.69-2.59 (m, 1H), 2.46-2.20 (m, 1H), 2.06 (dd, J=22.8, 10.4 Hz, 2H), 1.71-1.58 (m, 1H); LC-MS: m/z 436.1 (MH+).

Preparation: (1,1-dioxo-1,4-thiazinan-4-yl)-[4-nitro-2-[2-(trifluoromethyl)morpholin-4-yl]phenyl]methanone

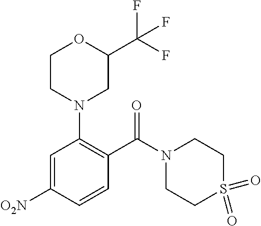 [SMILES: O=C(c1ccc([N+](=O)[O-])cc1N1CCOC(C(F)(F)F)C1)N1CCS(=O)(=O)CC1]

The synthesis of the title compound was effected analogously to the synthesis of (1,1-dioxo-1,4-thiazinan-4-yl)-[4-nitro-2[3-(trifluoromethyl)pyrrolidin-1-yl]phenyl]methanone wherein 2-(trifluoromethyl)morpholine hydrochloride was used instead of 3-(trifluoromethyl)pyrrolidine hydrochloride. The title compound was obtained in a yield of 15% (50 mg).

1H NMR (400 MHz, DMSO-d6) δ 8.01 (ddd, J=8.4, 2.2, 1.2 Hz, 1H), 7.92 (dd, J=5.5, 2.2 Hz, 1H), 7.67 (dd, J=8.3, 2.5 Hz, 1H), 4.56-4.28 (m, 2H), 4.14-3.93 (m, 1H), 3.81-3.42 (m, 5H), 3.29-3.07 (m, 5H), 2.99-2.71 (m, 2H); LC-MS: m/z 438.1 (MH+).
Preparation: [2-(3-azabicyclo[2.2.1]heptan-3-yl)-4-nitrophenyl]-(1,1-dioxo-1,4-thiazinan-4-yl)methanone

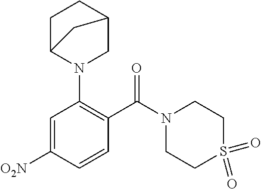 [SMILES: O=C(c1ccc([N+](=O)[O-])cc1N1CC2CCC1C2)N1CCS(=O)(=O)CC1]

The synthesis of the title compound was effected analogously to the synthesis of (1,1-dioxo-1,4-thiazinan-4-yl)-[4-nitro-2[3-(trifluoromethyl)pyrrolidin-1-yl]phenyl]methanone wherein 2-azabicyclo[2.2.1]heptane was used instead of 3-(trifluoromethyl)pyrrolidine hydrochloride. The title compound was obtained in a yield of 73% (273 mg).
LC-MS: m/z 380.2 (MH+).
   Preparation: [2-[(anti)-2,6-dimethylmorpholin-4-yl]-4-nitrophenyI]-(1,1-dioxo-1,4-thiazinan-4-yl)methanone

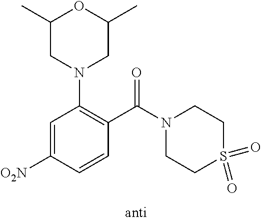 [SMILES: CC1CN(c2cc([N+](=O)[O-])ccc2C(=O)N2CCS(=O)(=O)CC2)CC(C)O1]

The synthesis of the title compound was effected analogously to the synthesis of (1,1-dioxo-1,4-thiazinan-4-yl)-[4-nitro-2[3-(trifluoromethyl)pyrrolidin-1-yl]phenyl]methanone wherein trans-2,6-dimethylmorpholine was used instead of 3-(trifluoromethyl)pyrrolidine hydrochloride. The title compound was obtained in a yield of 87% (3-[4mg) as a racemic mixture.

1H NMR (400 MHz, DMSO-d6) δ 7.95 (ddd, J=20.0, 8.3, 2.1 Hz, 1H), 7.81 (dd, J=13.0, 2.2 Hz, 1H), 7.69 (t, J=8.9 Hz, 1H), 4.70 (t, J=14.9 Hz, 1H), 4.01 (q, J=7.2, 4.9 Hz, 2H), 3.61-3.32 (m, 4H), 3.26-3.11 (m, 2H), 3.03-2.87 (m, 4H), 2.65 (dd, J=11.7, 5.9 Hz, 1H), 1.16 (dd, J=11.8, 6.3 Hz, 6H); LC-MS: m/z 398.4 (MH+).

Preparation: [2-(5-azaspiro[2.5]octan-5-yl)-4-nitrophenyl]-(1,1-dioxo-1,4-thiazinan-4-yl)methanone

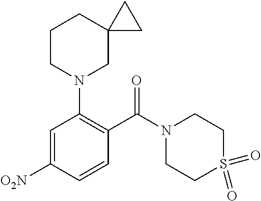 [SMILES: O=C(c1ccc([N+](=O)[O-])cc1N1CCCC2(CC2)C1)N1CCS(=O)(=O)CC1]

The synthesis of the title compound was effected analogously to the synthesis of (1,1-dioxo-1,4-thiazinan-4-yl)-[4-nitro-2-[3-(trifluoromethyl)pyrrolidin-1-yl]phenyl]methanone wherein 5-azaspiro[2.5]octane hydrochloride was used instead of 3-(trifluoromethyl)pyrrolidine hydrochloride. The title compound was obtained in a yield of 90% (234mg).

1H NMR (400 MHz, DMSO-d6) δ 7.92 (dd, J=8.3, 2.2 Hz, 1H), 7.78 (d, J=2.2 Hz, 1H), 7.55 (d, J=8.3 Hz, 1H), 4.31 (d, J=14.2 Hz, 1H), 3.83 (dd, J=13.7, 9.0 Hz, 1H), 3.55-3.36 (m, 3H), 3.30-3.07 (m, 4H), 2.95 (d, J=11.5 Hz, 1H), 2.90-2.80 (m, 1H), 2.60 (d, J=11.5 Hz, 1H), 1.68 (s, 2H), 1.48-1.29 (m, 2H), 0.35 (ddt, J=13.2, 8.8, 5.1 Hz, 4H); LC-MS: m/z 394.2 (MH+).

Preparation: (1,1-dioxo-1,4-thiazinan-4-yl)-[4-nitro-2-(4-oxa-7-azaspiro[2.5]octan-7-yl)phenyl]methanone

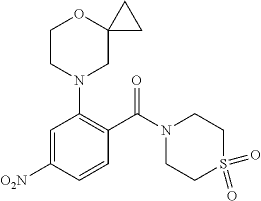 [SMILES: O=C(c1ccc([N+](=O)[O-])cc1N1CCOC2(CC2)C1)N1CCS(=O)(=O)CC1]

The synthesis of the title compound was effected analogously to the synthesis of (1,1-dioxo-1,4-thiazinan-4-yl)-[4-nitro-2-[3-(trifluoromethyl)pyrrolidin-1-yl]phenyl]methanone wherein 4-oxa-7-azaspiro[2.5]octane hydrochloride was used instead of 3-(trifluoromethyl)pyrrolidine hydrochloride. The title compound was obtained in a yield of 88% (231mg).

1H NMR (400 MHz, DMSO-d6) δ 7.96 (dd, J=8.4, 2.2 Hz, 1H), 7.83 (d, J=2.2 Hz, 1H), 7.61 (d, J=8.4 Hz, 1H), 4.35 (d, J=14.4 Hz, 1H), 3.83-3.69 (m, 3H), 3.61-3.35 (m, 4H), 3.25-3.04 (m, 4H), 2.96-2.82 (m, 2H), 0.78-0.45 (m, 4H); LC-MS: m/z 396.1 (MH+).

Preparation: [2-(3,3-difluoropiperidin-1-yl )-4-nitrophenyl]-(1,1-dioxo-1,4-thiazinan-4-yl)methanone

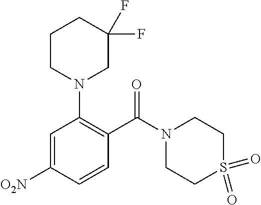 [SMILES: O=C(c1ccc([N+](=O)[O-])cc1N1CCCC(F)(F)C1)N1CCS(=O)(=O)CC1]

The synthesis of the title compound was effected analogously to the synthesis of (1,1-dioxo-1,4-thiazinan-4-yl)-[4-nitro-2[3-(trifluoromethyl)pyrrolidin-1-yl ]phenyl]methanone wherein 3,3-difluoropiperidine;hydrochloride was used instead of 3-(trifluoromethyl)pyrrolidine hydrochloride. The title compound was obtained in a yield of 26% (106 mg).

1H NMR (400 MHz, DMSO-d6) δ 8.01 (dd, J=8.3, 2.2 Hz, 1H), 7.91 (d, J=2.2 Hz, 1H), 7.59 (d, J=8.4 Hz, 1H), 4.31-4.23 (m, 1H), 3.82 (t, J=11.1 Hz, 1H), 3.57-3.43 (m, 2H), 3.30 (s, 2H), 3.24-3.10 (m, 4H), 3.00 (ddd, J=16.9, 6.2, 3.2 Hz, 2H), 1.99 (s, 2H), 1.75 (ddd, J=10.5, 6.7, 3.8 Hz, 2H); LC-MS: 404.1 (MH+).

Preparation: (1,1-dioxo-1,4-thiazinan-4-yl)-[4-nitro-2-(2-oxa-5-azabicyclo[2.2.1]heptan-5-yl)phenyl]methanone

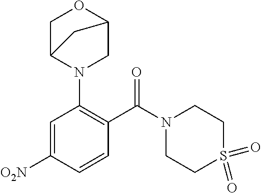 [SMILES: O=C(c1ccc([N+](=O)[O-])cc1N1CC2CC1CO2)N1CCS(=O)(=O)CC1]

The synthesis of the title compound was effected analogously to the synthesis of (1,1-dioxo-1,4-thiazinan-4-yl)-[4-nitro-2[3-(trifluoromethyl)pyrrolidin-1-yl]phenyl]methanone wherein 2-Oxa-5-azabicyclo[2.2.1]heptane was used instead of 3-(trifluoromethyl)pyrrolidine hydrochloride. The title compound was obtained in a yield of 81% (305 mg) as a mixture of diasteroisomers.

1H NMR (400 MHz, DMSO) 6 7.64-7.41 (m, 3H), 4.53 (dd, J=39.7, 14.5 Hz, 3H), 3.97-3.37 (m, 6H), 3.19-2.62 (m, 3H), 2.04-1.75 (m, 2H), 1.33-1.05 (m, 2H); LC-MS: m/z 382.36 (MH+).

Preparation: (1,1-dioxo-1,4-thiazinan-4-yl)-[4-nitro-2-(6-oxa-3-azabicyclo[3.1.1]heptan yl)phenyl]methanone

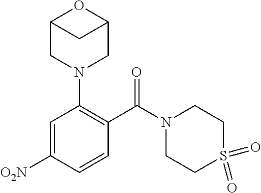 [SMILES: O=C(c1ccc([N+](=O)[O-])cc1N1CC2CC(C1)O2)N1CCS(=O)(=O)CC1]

The synthesis of the title compound was effected analogously to the synthesis of (1,1-dioxo-1,4-thiazinan-4-yl)-[4-nitro-2-[3-(trifluoromethyl)pyrrolidin-1-yl]phenyl]methanone wherein 6-oxa-3-azabicyclo[3.1.1]heptane hydrochloride was used instead of 3-(trifluoromethyl)pyrrolidine hydrochloride. The title compound was obtained in a yield of 25% 95 mg).

1H NMR (400 MHz, DMSO) 6 7.67 (d, J=1.7 Hz, 1H), 7.59 (d, J=2.3 Hz, 2H), 4.60 (dd, J=52.2, 10.7 Hz, 3H), 3.85-3.37 (m, 10H), 3.09 (d, J=7.8 Hz, 2H), 1.16 (s, 1H); LC-MS: m/z 382.3 (MH+).

Preparation: (1,1-dioxo-1,4-thiazinan-4-yl)-[4-nitro-2-(8-oxa-3-azabicyclo[3.2.1]octan-3-yl)phenyl]methanone

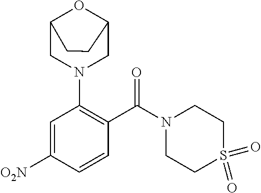 [SMILES: O=C(c1ccc([N+](=O)[O-])cc1N1CC2CCC(C1)O2)N1CCS(=O)(=O)CC1]

The synthesis of the title compound was effected analogously to the synthesis of (1,1-dioxo-1,4-thiazinan-4-yl)-[4-nitro-2-[3-(trifluoromethyl)pyrrolidin-1-yl]phenyl]methanone wherein 8-oxa-3-azabicyclo[3.2.1]octane hydrochloride was used instead of 3-(trifluoromethyl)pyrrolidine hydrochloride. The title compound was obtained in a yield of 25% (95 mg).

1H NMR (400 MHz, DMSO-d6) δ 7.91 (dd, J=8.3, 2.2 Hz, 1H), 7.78 (d, J=2.2 Hz, 1H), 7.68 (d, J=8.3 Hz, 1H), 4.76 (d, J=13.2 Hz, 1H), 4.35 (d, J=39.1 Hz, 2H), 3.62-3.34 (m, 4H), 3.27-3.07 (m, 4H), 2.89 (td, J =27.3, 24.1, 12.7 Hz, 3H), 1.83 (s, 3H), 1.70 (d, J=8.9 Hz, 1H); LC-MS: m/z 396.1 (MH+).

Preparation: [2-(3-azabicyclo[3.1.1]heptan-3-yl )-4-nitrophenyl]-(1,1-dioxo-1,4-thiazinan-4-yl)methanone

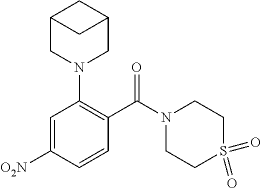 [SMILES: O=C(c1ccc([N+](=O)[O-])cc1N1CC2CC(C2)C1)N1CCS(=O)(=O)CC1]

The synthesis of the title compound was effected analogously to the synthesis of (1,1-dioxo-1,4-thiazinan-4-yl)-[4-nitro-2-[3-(trifluoromethyl)pyrrolidin-1-yl]phenyl]methanone wherein 3-azabicyclo[3.1.1]heptane hydrochloride was used instead of 3-(trifluoromethyl)pyrrolidine hydrochloride. The title compound was obtained in a yield of 58% (145 mg).

### LC-MS: m/z 380.36 (MH+).

Preparation: [2-(4,4-difluoropiperidin-1-yl)-4-nitrophenyl]-(1,1-dioxo-1,4-thiazinan-4-yl)methanone

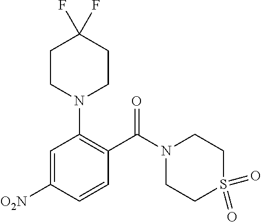 [SMILES: O=C(c1ccc([N+](=O)[O-])cc1N1CCC(F)(F)CC1)N1CCS(=O)(=O)CC1]

The synthesis of the title compound was effected analogously to the synthesis of (1,1-dioxo-1,4-thiazinan4-yl)-[4-nitro-2-[3-(trifluoromethyl)pyrrolidin-1-yl]phenyl]methanone wherein 4,4-difluoropiperidine was used instead of 3-(trifluoromethyl)pyrrolidine hydrochloride. The title compound was obtained in a yield of 54% (215 mg).

1H NMR (400 MHz, DMSO-d6) δ 7.96 (dd, J=8.3, 2.2 Hz, 1H), 7.88 (d, J=2.2 Hz, 1H), 7.64 (d, J=8.4 Hz, 1H), 4.49 (dd, J=12.1, 6.8 Hz, 1H), 3.71 (ddd, J=13.7, 10.2, 2.9 Hz, 1H), 3.65-3.42 (m, 2H), 3.30 (s, 3H), 3.25-2.88 (m, 5H), 2.15-1.97 (m, 4H); LC-MS: m/z 404.1 (MH+).

Preparation: (1,1-dioxo-1,4-thiazinan-4-yl)-[4-nitro-2-piperidin-1-ylphenyl)methanone

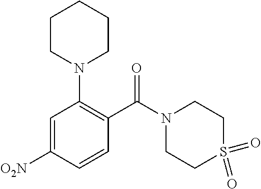 [SMILES: O=C(c1ccc([N+](=O)[O-])cc1N1CCCCC1)N1CCS(=O)(=O)CC1]

The synthesis of the title compound was effected analogously to the synthesis of (1,1-dioxo-1,4-thiazinan-4-yl)-[4-nitro-2-[-b 3-(trifluoromethyl)pyrrolidin-1-yl]phenyl]methanone wherein piperidine was used instead of 3-(trifluoromethyl)pyrrolidine hydrochloride. The title compound was obtained in a yield of 97% (177 mg).

1H NMR (400 MHz, DMSO-d6) δ 7.91 (dd, 1H), 7.79 (d, 1H), 7.56 (d, 1H), 4.33 (d, 1H), 3.81 (dd, 1H), 3.55-b 3.47 (m, 2H), 3.38 (t, 1H), 3.19-3.01 (m, 5H), 2.90-2.80 (m, 2H), 1.66-1.49 (m, 6H); LC-MS: 368.19 (MH+).

Preparation: (1,1-dioxo-1,4-thiazinan-4-yl)-(2-morpholin-4-yl-4-nitrophenyl)methanone

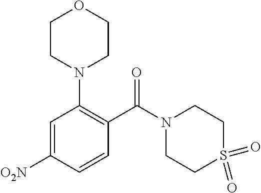 [SMILES: O=C(c1ccc([N+](=O)[O-])cc1N1CCOCC1)N1CCS(=O)(=O)CC1]

The synthesis of the title compound was effected analogously to the synthesis of (1,1-dioxo-1,4-thiazinan-4-yl)-[4-nitro-2-[3-(trifluoromethyl)pyrrolidin-1-yl]phenyl]methanone wherein morpholine was used instead of 3-(trifluoromethyl)pyrrolidine hydrochloride. The title compound was obtained in a yield of 50% (121 mg).

1H NMR (400 MHz, DMSO-d6) δ 7.95 (dd, J=8.3, 2.2 Hz, 1H), 7.83 (d, J=2.2 Hz, 1H), 7.61 (d, J=8.3 Hz, 1H), 4.35 (d, J=13.9 Hz, 1H), 3.84-3.75 (m, 1H), 3.74-3.62 (m, 4H), 3.55 (t, J=5.7 Hz, 2H), 3.39 (t, J =10.2 Hz, 1H), 3.25-3.11 (m, 4H), 3.09-3.01 (m, 1H), 2.94-2.83 (m, 2H); LC-MS: m/z 370.2 (MH+).

Preparation: [4-amino-2-[3-(trifluoromethyl)pyrrolidin-1-yl ]phenyl]-(1,1-dioxo-1,4-thiazinan-4-yl)methanone

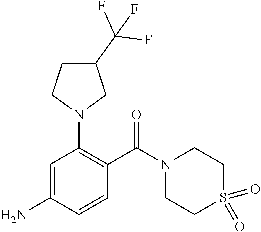 [SMILES: Nc1ccc(C(=O)N2CCS(=O)(=O)CC2)c(N2CCC(C(F)(F)F)C2)c1]

A mixture of (1,1-dioxo-1,4-thiazinan-4-yl)-[4-nitro-2-[3-(trifluoromethyl)pyrrolidin-1-yl]phenyl]methanone (209.0 mg, 0.500 mmol) and Pd-C 10% (52.78 mg, 0.050 mmol) was stirred under H₂ atmosphere (1 atm) at RT overnight. After this time the reaction was filtered and concentrated under vacuum. The residue was purified by SCX cartridge first washing with MeOH and then eluting with NH₃ 1M in MeOH. The basic fractions were evaporated affording the product of formula [4-amino-2-[3-(trifluoromethyl)pyrrolidin-1-yl]phenyl]-(1,1-dioxo-1,4-thiazinan-4-yl)methanone.
Yield: 136 mg

1H NMR (400 MHz, DMSO-d6) δ 6.88 (d, J=8.1 Hz, 1H), 6.03 (d, J=18.2 Hz, 2H), 5.24 (d, J=7.5 Hz, 2H), 4.23 (s, 1H), 3.66 (s, 4H), 3.31-2.92(m, 8H), 2.17 (dq, J=12.9, 6.2 Hz, 1H), 1.96 (dq, J=13.9, 7.0 Hz, 1H); LC-MS: m/z 392.1.

Preparation [4-amino-2-(3,3-difluoropyrrolidin-1-yl)phenyl]-(1,1-dioxo-1,4-thiazinan-4-yl)methanone:

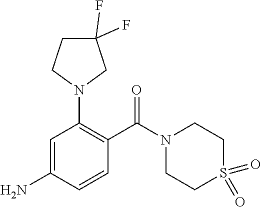 [SMILES: Nc1ccc(C(=O)N2CCS(=O)(=O)CC2)c(N2CCC(F)(F)C2)c1]

The synthesis of the title compound was effected analogously to the synthesis of [4-amino-2-[3-(trifluoromethyl)pyrrolidin-1-yl]phenyl]-(1,1-dioxo-1,4-thiazinan-4-yl)methanone wherein [2-(3,3-difluoropyrrolidin-1-yl)-4-nitrophenyl]-(1,1-dioxo-1,4-thiazinan-4-yl)methanone was used instead of 1,1-dioxo-1,4-thiazinan-4-yl)-[4-nitro-2-[3-(trifluoromethyl)pyrrolidin-1-yl]phenyl]methanone. The title compound was obtained in a yield of 87% (50 mg).

1H NMR (400 MHz, DMSO-d6) δ 6.91 (d, 1H), 6.10 (dd, 1H), 6.01 (d, 1H), 5.32 (s, 2H), 3.70 (d, 2H), 3.55-3.00 (m, 8H), 2.45-2.31 (m, 2H), 1.77 (s, 2H); LC-MS: 360.1 (MH+).

Preparation: [4-amino-2-[3-(trifluoromethyppiperidin-1-yl]phenyl]-(1,1-dioxo-1,4-thiazinan-4-yl)methanone

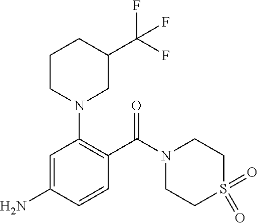 [SMILES: Nc1ccc(C(=O)N2CCS(=O)(=O)CC2)c(N2CCCC(C(F)(F)F)C2)c1]

The synthesis of the title compound was effected analogously to the synthesis of [4-amino-2-[3-(trifluoromethyl)pyrrolidin-1-yl]phenyl]-(1,1-dioxo-1,4-thiazinan-4-yl)methanone wherein (1,1-dioxo-1,4-thiazinan-4-yl)-[4-nitro-2-[3-(trifluoromethyppiperidin-1-yl]phenyl]methanone was used instead of 1,1-dioxo-1,4-thiazinan-4-yl)-[4-nitro-2-[3-(trifluoromethyl)pyrrolidin-1-yl]phenyl]methanone. The title compound was obtained in a yield of 76% (181 mg).

1H NMR (400 MHz, DMSO-d6) δ 6.93 (d, 1H), 6.32-6.25 (m, 2H), 5.36 (s, 2H), 4.35 (s, 3H), 3.57 (s, 4H), 3.30 (s, 4H), 2.37 (d, 1H), 1.83 (dd, 2H), 1.61— 1.08 (m, 2H), 0.85 (d, 1H); LC-MS: 406.2 (MH+).

Preparation: [4-amino-2-[2-(trifluoromethyl)morpholin-4-yl]phenyl]-(1,1-dioxo-1,4-thiazinan-4-yl)methanone

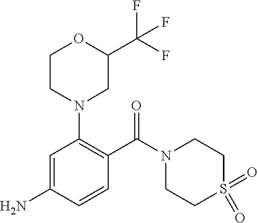 [SMILES: Nc1ccc(C(=O)N2CCS(=O)(=O)CC2)c(N2CCOC(C(F)(F)F)C2)c1]

The synthesis of the title compound was effected analogously to the synthesis of [4-amino-2-[3-(trifluoromethyl)pyrrolidin-1-yl]phenyl]-(1,1-dioxo-1,4-thiazinan-4-yl)methanone wherein (1,1-dioxo-1,4-thiazinan-4-yl)-[4-nitro-2-[2-(trifluoromethyl)morpholin-4-yl]phenyl]methanone was used instead of 1,1-dioxo-1,4-thiazinan-4-yl)-[4-nitro-2-[3-(trifluoromethyl)pyrrolidin yl]phenyl]methanone. The title compound was obtained in a yield of 82% (38 mg).

### LC-MS: m/z 370.2 (MH+).

Preparation: [4-amino-2-(3-azabicyclo[2.2.1]heptan-3-yl)phenyl]-(1,1-dioxo-1,4-thiazinan-4-yl)methanone

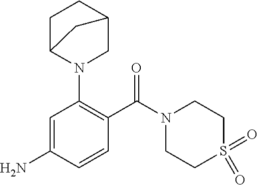 [SMILES: Nc1ccc(C(=O)N2CCS(=O)(=O)CC2)c(N2CC3CCC2C3)c1]

The synthesis of the title compound was effected analogously to the synthesis of [4-amino-2-[3-(trifluoromethyl)pyrrolidin-1-yl]phenyl]-(1,1-dioxo-1,4-thiazinan-4-yl)methanone wherein [2-(3-azabicyclo[2.2.1]heptan-3-y0-4-nitrophenyl]-(1,1-dioxo-1,4-thiazinan-4-yl)methanone was used instead of 1,1-dioxo-1,4-thiazinan-4-yl)-[4-nitro-2-[3-(trifluoromethyl)pyrrolidin-1-yl]phenyl]methanone. The title compound was obtained in a yield of 78% (195 mg).

### LC-MS: m/z 350.3 (MH+)

Preparation: [4-amino-2-[(anti)-2,6-dimethylmorpholin-4-yl]phenyI]-(1,1-dioxo-1,4-thiazinan-4-yl)methanone

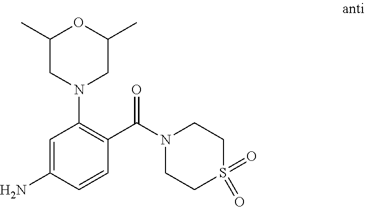 [SMILES: CC1CN(c2cc(N)ccc2C(=O)N2CCS(=O)(=O)CC2)CC(C)O1]

The synthesis of the title compound was effected analogously to the synthesis of [4-amino-2-[3-(trifluoromethyl)pyrrolidin-1-yl]phenyl]-(1,1-dioxo-1,4-thiazinan-4-yl)methanone wherein [2-[(anti)-2,6-dimethylmorpholin-4-yl]-4-nitrophenyI]-(1,1-dioxo-1,4-thiazinan-4-yl)methanonewas was used instead of 1,1-dioxo-1,4-thiazinan-4-yl)-[4-nitro-2-[3-(trifluoromethyl)pyrrolidin-1-yl]phenyl]methanone. The title compound was obtained in a yield of 47% (80 mg) as a racemic mixture.

1H NMR (400 MHz, DMSO-d6) δ 6.94 (d, J=8.3 Hz, 1H), 6.25 (d, J=7.1 Hz, 2H), 5.33 (s, 2H), 4.59 (s, 1H), 3.94 (s, 2H), 3.66 (s, 1H), 3.24-2.62 (m, 10H), 1.14 (d, J=6.5 Hz, 6H); LC-MS: m/z 368.4 (MH+).

Preparation: [4-amino-2-(5-azaspiro[2.5]octan-5-yl)phenyl]-(1,1-dioxo-1,4-thiazinan-4-yl)methanone

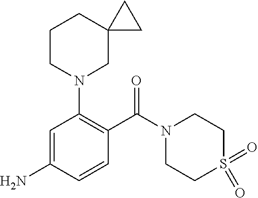 [SMILES: Nc1ccc(C(=O)N2CCS(=O)(=O)CC2)c(N2CCCC3(CC3)C2)c1]

The synthesis of the title compound was effected analogously to the synthesis of the [4-amino-2-[3-(trifluoromethyl)pyrrolidin-1-yl]phenyl]-(1,1-dioxo-1,4-thiazinan-4-yl)methanone wherein [2-(5-azaspiro[2.5]octan-5-yl)-4-nitrophenyI]-(1,1-dioxo-1,4-thiazinan-4-yl)methanone was used instead of 1,1-dioxo-1,4-thiazinan-4-yl)-[4-nitro-2-[3-(trifluoromethyl)pyrrolidin-1-yl]phenyl]methanone. The title compound was obtained in a yield of 63% (136 mg).

1H NMR (400 MHz, DMSO-d6) δ 6.85 (d, J=7.9 Hz, 1H), 6.22 (d, J=7.8 Hz, 2H), 5.30 (s, 2H), 4.08 (d, J =5.9 Hz, 2H), 3.55 (s, 2H), 3.12 (dd, J=40.5, 16.0 Hz, 5H), 2.77-2.61 (m, 2H), 2.42 (d, J=7.3 Hz, 1H), 1.63 (s, 2H), 1.34 (s, 2H), 0.31 (d, J=10.0 Hz, 4H); LC-MS: m/z 364.2 (MH+).

Preparation: [4-amino-2-(4-oxa-7-azaspiro[2.5]octan-7-yl)phenyl]-(1,1-dioxo-1,4-thiazinan-4-yl)methanone

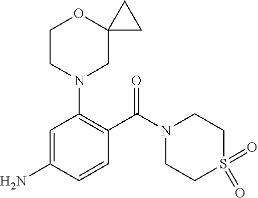 [SMILES: Nc1ccc(C(=O)N2CCS(=O)(=O)CC2)c(N2CCOC3(CC3)C2)c1]

The synthesis of the title compound was effected analogously to the synthesis of [4-amino-2-[3-(trifluoromethyl)pyrrolidin-1-yl]phenyl]-(1,1-dioxo-1,4-thiazinan-4-yl)methanone wherein (1,1-dioxo-1,4-thiazinan-4-yl)-[4-nitro-2-(4-oxa-7-azaspiro[2.5]octan-7-yl)phenyl]methanone was used instead of 1,1-dioxo-1,4-thiazinan-4-yl)-[4-nitro-2-[3-(trifluoromethyl)pyrrolidin-1-yl]phenyl]methanone. The title compound was obtained in a yield of 11% (25 mg).

1H NMR (400 MHz, DMSO-d6) δ 6.93-6.86 (m, 1H), 6.29-6.22 (m, 2H), 5.34 (d, J=7.3 Hz, 2H), 4.13 (s, 1H), 3.85 (s, 1H), 3.73-3.42 (m, 4H), 3.24-2.85 (m, 6H), 2.80-2.63 (m, 2H), 0.60 (d, J=71.4 Hz, 4H); LC-MS: m/z 366.2 (MH+).

Preparation: [4-amino-2-(3,3-difluoropiperidin-1-yl)phenyI]-(1,1-dioxo-1,4-thiazinan-4-yl)methanone

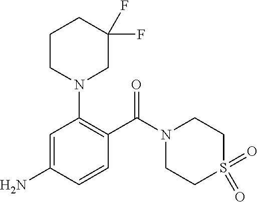 [SMILES: Nc1ccc(C(=O)N2CCS(=O)(=O)CC2)c(N2CCCC(F)(F)C2)c1]

The synthesis of the title compound was effected analogously to the synthesis of the [4-amino-2-[3-(trifluoromethyl)pyrrolidin-1-yl]phenyl]-(1,1-dioxo-1,4-thiazinan-4-yl)methanone wherein [2-(3,3-difluoropiperidin-1-yl)-4-nitrophenyI]-(1,1-dioxo-1,4-thiazinan-4-yl)methanone was used instead of 1,1-dioxo-1,4-thiazinan-4-yl)-[4-nitro-2-[3-(trifluoromethyl)pyrrolidin-1-yl]phenyl]methanone. The title compound was obtained in a yield of 96% (94 mg).

1H NMR (400 MHz, DMSO-d6) δ 6.89 (d, J=8.6 Hz, 1H), 6.29 (dq, J=4.3, 2.0 Hz, 2H), 5.37 (d, J=7.2 Hz, 2H), 4.07 (d, J=7.7 Hz, 1H), 3.83 (s, 1H), 3.51 (s, 2H), 3.24 (d, J=49.7 Hz, 2H), 2.91 (d, J=102.7 Hz, 6H), 1.97 (td, J=13.8, 6.4 Hz, 2H), 1.70 (s, 2H); LC-MS: m/z 374.1 (MH+).

Preparation: [4-amino-2-(2-oxa-5-azabicyclo[2.2.1]heptan-5-yl)phenyl]-(1,1-dioxo-1,4-thiazinan-4-yl)methanone

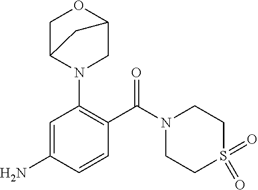 [SMILES: Nc1ccc(C(=O)N2CCS(=O)(=O)CC2)c(N2CC3CC2CO3)c1]

The synthesis of the title compound was effected analogously to the synthesis of [4-amino-2-[3-(trifluoromethy)pyrrolidin-1-yl]phenyl]-(1,1-dioxo-1,4-thiazinan-4-yl)methanone wherein (1,1-dioxo-1,4-thiazinan-4-yl)-[4-nitro-2-(2-oxa-5-azabicyclo[2.2.1]heptan-5-yl)phenyl]methanone was used instead of 1,1-dioxo-1,4-thiazinan-4-yl)-[4-nitro-2-[3-(trifluoromethyl)pyrrolidin-1-yl]phenyl]methanone. The title compound was obtained in a yield of 100% (283 mg) as a mixture of diasteroisomers.

1H NMR (400 MHz, DMSO) 6 6.87 (d, J=8.2 Hz, 1H), 6.18-5.83 (m, 2H), 5.18 (s, 2H), 4.49 (s, 1H), 4.22 (s, 2H), 4.14-3.43 (m, 6H), 3.25-3.13 (m, 3H), 3.03 (s, 2H), 1.80 (s, 2H); LC-MS: m/z 352.36.

Preparation: [4-amino-2-(6-oxa-3-azabicyclo[3.1.1]heptan-3-yl)phenyl]-(1,1-dioxo-1,4-thiazinan-4-yl)methanone

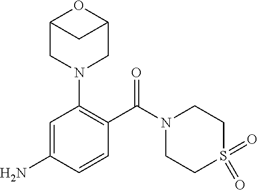 [SMILES: Nc1ccc(C(=O)N2CCS(=O)(=O)CC2)c(N2CC3CC(C2)O3)c1]

The synthesis of the title compound was effected analogously to the synthesis of [4-amino-2-[3-(trifluoromethyl)pyrrolidin-1-yl]phenyl]-(1,1-dioxo-1,4-thiazinan-4-yl)methanone wherein (1,1-dioxo-1,4-thiazinan-4-yl)-[4-nitro-2-(6-oxa-3-azabicyclo[3.1.1]heptan-3-yl)phenyl]methanone was used instead of 1,1-dioxo-1,4-thiazinan-4-yl)-[4-nitro-2-[3-(trifluoromethyl)pyrrolidin-1-yl]phenyl]methanone. The title compound was obtained in a yield of 70% (58 mg).

1H NMR (400 MHz, DMSO) δ 6.94 (dd, J=29.9, 8.2 Hz, 1H), 6.39-6.03 (m, 2H), 4.45 (dd, J=99.7, 5.5 Hz, 3H), 3.83 (s, 1H), 3.69-3.51 (m, 3H), 3.24-2.93 (m, 9H), 2.08 (s, 2H); LC-MS: m/z 352.36 (MH+).

Preparation: [4-amino-2-(8-oxa-3-azabicyclo[3.2.1]octan-3-yl)phenyI]-(1,1-dioxo-1,4-thiazinan yl)methanone

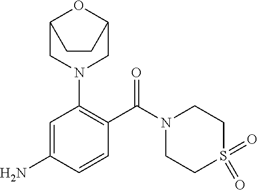 [SMILES: Nc1ccc(C(=O)N2CCS(=O)(=O)CC2)c(N2CC3CCC(C2)O3)c1]

The synthesis of the title compound was effected analogously to the synthesis of [4-amino-2-[3-(trifluoromethyl)pyrrolidin-1-yl]phenyl]-(1,1-dioxo-1,4-thiazinan-4-yl)methanone wherein (1,1-dioxo-1,4-thiazinan-4-yl)-[4-nitro-2-(8-oxa-3-azabicyclo[3.2.1]octan-3-yl)phenyl]methanone was used instead of 1,1-dioxo-1,4-thiazinan-4-yl)-[4-nitro-2-[3-(trifluoromethyl)pyrrolidin-1-yl]phenyl]methanone. The title compound was obtained in a yield of 68% (185 mg). LC-MS: m/z 366.2 (MH+).

Preparation: [4-amino-2-(3-azabicyclo[3.1.1]heptan-3-yl)phenyl]-(1,1-dioxo-1,4-thiazinan-4-yl)methanone

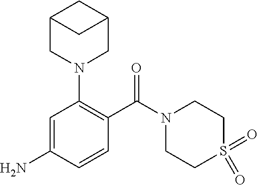 [SMILES: Nc1ccc(C(=O)N2CCS(=O)(=O)CC2)c(N2CC3CC(C3)C2)c1]

The synthesis of the title compound was effected analogously to the synthesis of [4-amino-2-[3-(trifluoromethyl)pyrrolidin-1-yl]phenyl]-(1,1-dioxo-1,4-thiazinan-4-yl)methanone wherein [2-(3-azabicyclo[3.1.1]heptan-3-yl)-4-nitrophenyl]-(1,1-dioxo-1,4-thiazinan-4-yl)methanone was used instead of 1,1-dioxo-1,4-thiazinan-4-yl)-[4-nitro-2-[3-(trifluoromethyl)pyrrolidin-1-yl]phenyl]methanone. The title compound was obtained in a yield of 100% (140 mg).

1H NMR (400 MHz, DMSO) δ 6.85 (d, J=8.1 Hz, 1H), 6.16 (d, J=2.0 Hz, 1H), 6.03 (dd, J=8.1, 1.9 Hz, 1H), 5.16 (s, 2H), 4.43 (s, 2H), 3.95-3.34 (m, 9H), 3.02 (d, J=13.9 Hz, 1H), 2.46 (t, J=6.1 Hz, 2H), 2.15-1.98 (m, 2H), 1.35 (dd, J=6.3, 2.8 Hz, 2H); LC-MS: m/z 350.2 (MH+).

Preparation: [4-amino-2-(4,4-difluoropiperidin-1-yl)phenyl]-(1,1-dioxo-1,4-thiazinan-4-yl)methanone

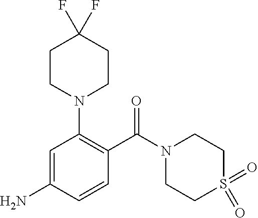 [SMILES: Nc1ccc(C(=O)N2CCS(=O)(=O)CC2)c(N2CCC(F)(F)CC2)c1]

The synthesis of the title compound was effected analogously to the synthesis of [4-amino-2-[3-(trifluoromethyl)pyrrolidin-1-yl]phenyl]-(1,1-dioxo-1,4-thiazinan-4-yl)methanone wherein [2-(4,4-difluoropiperidin-1-yl)-4-nitrophenyl]-(1,1-dioxo-1,4-thiazinan-4-yl)methanone was used instead of 1,1-dioxo-1,4-thiazinan-4-yl)-[4-nitro-2-[3-(trifluoromethyl)pyrrolidin-1-yl]phenyl]methanone. The title compound was obtained in a yield of 87% (174 mg).

1H NMR (400 MHz, DMSO-d6) δ 6.93 (d, J=8.1 Hz, 1H), 6.32-6.23 (m, 2H), 5.35 (d, J=7.2 Hz, 2H), 4.44-4.10 (m, 1H), 3.54 (d, J=66.5 Hz, 3H), 3.32-2.70 (m, 8H), 2.01 (tt, J=13.6, 5.5 Hz, 4H); LC-MS: m/z 374.1 (MH+).

Preparation: (4-amino-2-piperidin-1-ylphenyl)-(1,1-dioxo-1,4-thiazinan-4-yl)methanone

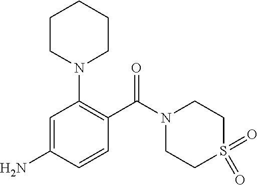 [SMILES: Nc1ccc(C(=O)N2CCS(=O)(=O)CC2)c(N2CCCCC2)c1]

The synthesis of the title compound was effected analogously to the synthesis of [4-amino-2-[3-(trifluoromethyl)pyrrolidin-1-yl]phenyl]-(1,1-dioxo-1,4-thiazinan-4-yl)methanone wherein (1,1-dioxo-1,4-thiazinan-4-yl)-[4-nitro-2-piperidin-1-yl phenyl)methanone was used instead of 1,1-dioxo-1,4-thiazinan-4-yl)[4-nitro-2-[3-(trifluoromethyl)pyrrolidin-1-yl ]phenyl]methanone. The title compound was obtained in a yield of 92% (150 mg).

1H NMR (400 MHz, DMSO-d6) 66.88 (d, 1H), 6.29-6.16 (m, 2H), 5.31 (s, 2H), 4.17-4.07 (m, 1H), 3.86 (s, 1H), 3.56 (s, 2H), 3.19-2.63 (m, 8H), 1.51 (d, 6H); LC-MS: m/z 338.17 (MH+).

Preparation: (4-amino-2-morpholin-4-ylphenyI)-(1,1-dioxo-1,4-thiazinan-4-yl)methanone

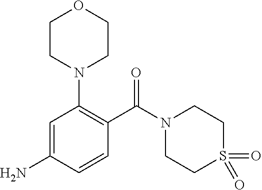 [SMILES: Nc1ccc(C(=O)N2CCS(=O)(=O)CC2)c(N2CCOCC2)c1]

The synthesis of the title compound was effected analogously to the synthesis [4-amino-2-[3-(trifluoromethyl)pyrrolidin-1-yl]phenyl]-(1,1-dioxo-1,4-thiazinan-4-yl)methanone wherein (1,1-dioxo-1,4-thiazinan-4-yl)-(2-morpholin-4-yl-4-nitrophenyl)methanone was used instead of 1,1-dioxo-1,4-thiazinan-4-yl)[4-nitro-2-[3-(trifluoromethyl)pyrrolidin-1-yl]phenyl]methanone. The title compound was obtained in a yield of 56% (62 mg).

1H NMR (400 MHz, DMSO-d6) δ 6.90 (d, J=8.6 Hz, 1H), 6.29-6.22 (m, 2H), 5.37 (s, 2H), 4.14 (s, 1H), 3.63 (s, 7H), 3.26-2.59 (m, 8H); LC-MS: m/z 340.4 (MH+).

Preparation: N-[4-(1,1-dioxo-1,4-thiazinane-4-carbonyl)-3-[3-(trifluoromethyl)pyrrolidin-1-yl]phenyl]cyclopropanecarboxamide

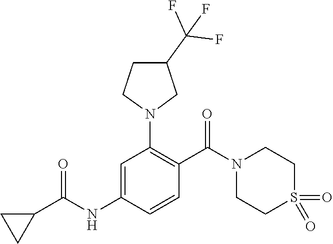 [SMILES: O=C(Nc1ccc(C(=O)N2CCS(=O)(=O)CC2)c(N2CCC(C(F)(F)F)C2)c1)C1CC1]

### Example 183a, 183b, 183c

To a mixture of [4-amino-2-[3-(trifluoromethyl)pyrrolidin-1-yl ]phenyl]-(1,1-dioxo-1,4-thiazinan yl)methanone (136.0 mg, 0.350 mmol) and N,N-Diisopropylethylamine (0.21 mL, 1.22 mmol) in DCM (3.892 mL) at 0° C., cyclopropanecarbonyl chloride (0.06 mL, 0.630 mmol) was added dropwise. The reaction was allowed to reach the RT and stirred at this temperature for 1 h. After this time the reaction was concentrated under vacuum and directly purified by FC on RP using basic conditions (eluting from H₂O +0.1% of ammonium hydroxide/ CH₃CN 95:5 to H₂O +0.1% of ammonium hydroxide/ CH₃CN 60:40) affording the product of formula N-[4-(1,1-dioxo-1,4-thiazinane-4-carbonyl)-3[3-(trifluoromethyl)pyrrolidin-1-yl]phenyl]cyclopropanecarboxamide as racemic mixture.
Yield: 95 mg

1H NMR (400 MHz, DMSO-d6) δ 10.18 (d, 1H), 7.21-7.13 (m, 2H), 7.04 (dd, 1H), 4.36 (s, 1H), 3.64 (ddt, 3H), 3.31 (s, 9H), 2.21 (s, 1H), 2.01 (dt, 1H), 1.75 (dt, 1H), 0.84-0.74 (m, 4H); LC-MS: 460.2 (MH+).

The racemic mixture (Example 183c) was then separated into single enantiomers by chiral semi-preparative HPLC

| | Column | Chiralpak AS-H (25 × 0.46 cm), 5μ | |
|---|---|---|---|
| | Mobile phase | n-Hexane/Ethanol 70/30% v/v | |
| | Flow rate (ml/min) | 17 ml/min | |
| | DAD detection | 220 nm | |
| Preparative chiral | Loop | 1000 μL | |
| chromatography | Total amount | 92 mg | |
| protocol: | Injection | 11.5 mg (each injection) | |
| 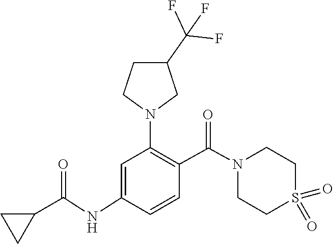 [SMILES: O=C(Nc1ccc(C(=O)N2CCS(=O)(=O)CC2)c(N2CCC(C(F)(F)F)C2)c1)C1CC1] | Enantiomer 1 | Rt 11.42 min | 100% ee |
| 3R or 3S enantiomer | LC-MS: m/z 460.2 (MH+) | | |
| | N-[4-(1,1-dioxo-1,4-thiazinane-4-carbonyl)-3-[3r or 3S-(tri- | | |
| | fluoromethyl)pyrrolidin-1-yl]phenyl]cyclopropanecarboxamide | | |
| | Example 183a | | |
| 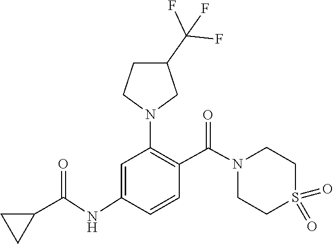 [SMILES: O=C(Nc1ccc(C(=O)N2CCS(=O)(=O)CC2)c(N2CCC(C(F)(F)F)C2)c1)C1CC1] | Enantiomer 2 | Rt 18.58 min | 100% ee |

| | |
|---|---|
| 3R or 3S enantiomer | LC-MS: m/z 460.2 (MH+) |
| | N-[4-(1,1-dioxo-1,4-thiazinane-4-carbonyl)-3-[3R or 3S-(tri- |
| | fluoromethyl)pyrrolidin-1-yl]phenyl]cyclopropanecarboxamide |
| | Example 183b |

Preparation: N-[3-(3,3-difluoropyrrolidin-1-yl )-4-(1,1-dioxo-1,4-thiazinane-4-carbonyl)phenyl]cyclopropanecarboxamide

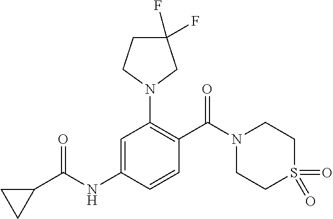 [SMILES: O=C(Nc1ccc(C(=O)N2CCS(=O)(=O)CC2)c(N2CCC(F)(F)C2)c1)C1CC1]

### Example 184

The synthesis of the title compound was effected analogously to the synthesis of the compound of Example 183a-c (N-[4-(1,1-dioxo-1,4-thiazinane-4-carbonyl)-3-[3-(trifluoromethyl)pyrrolidin-1-yl]phenyl]cyclopropanecarboxamide) wherein [4-amino-2-(3,3-difluoropyrrolidin-1-yl )phenyl]-(1,1-dioxo-1,4-thiazinan-4-yl)methanone was used instead of [4-amino-2-[3-(trifluoromethyl)pyrrolidin-1-yl ]phenyl]-(1,1-dioxo-1,4-thiazinan-4-yl)methanone. The title compound was obtained in a yield of 43% (29 mg).

1H NMR (400 MHz, DMSO-d6) δ 10.22 (s, 1H), 7.22-7.13 (m, 2H), 7.09 (dd, J=8.3, 1.8 Hz, 1H), 4.32 (d, J =14.2 Hz, 1H), 3.78-3.68 (m, 2H3.65-3.44 (m, 3H), 3.36 (t, J=7.2 Hz, 3H), 3.19 (d, J=10.5 Hz, 2H), 3.06 (d, J=14.0 Hz, 1H), 2.43 (dd, J=14.3, 7.2 Hz, 2H), 1.77 (tt, J=7.1, 5.5 Hz, 1H), 0.88-0.74 (m, 4H); LC-MS: m/z 428.2 (MH+).

Preparation: N-[4-(1,1-dioxo-1,4-thiazinane-4-carbonyl)-3-[3-(trifluoromethyl)piperidin-1-yl]phenyl]cyclopropanecarboxamide

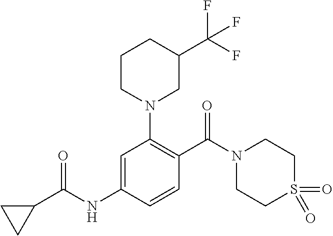 [SMILES: O=C(Nc1ccc(C(=O)N2CCS(=O)(=O)CC2)c(N2CCCC(C(F)(F)F)C2)c1)C1CC1]

### Example 185a, 185b, 185c

The synthesis of the title compound was effected analogously to the synthesis of the compound of Example 183a-c (N-[4-(1,1-dioxo-1,4-thiazinane-4-carbonyl)-3-[3-(trifluoromethyl)pyrrolidin-1-yl]phenyl]cyclopropanecarboxamide) wherein N-[4-(1,1-dioxo-1,4-thiazinane-4-carbonyl)-3-[3-(trifluoromethyppiperidin-1-yl ]phenyl]cyclopropanecarboxamide was used instead of [4-amino-2-[3-(trifluoromethyl)pyrrolidin-1-yl ]phenyl]-(1,1-dioxo-1,4-thiazinan-4-yl)methanone. The title compound was obtained in a yield of 25% (50 mg) as a racemic mixture.

1H NMR (400 MHz, DMSO-d6) δ 10.30 (d, J=5.7 Hz, 1H), 7.46 (d, J=29.1 Hz, 1H), 7.34-7.28(m, 1H), 7.22 (d, J=8.2 Hz, 1H), 4.38 (dd, J=35.1, 13.8 Hz, 1H), 3.79-3.42 (m, 4H), 3.29-2.88 (m, 6H), 2.78 (t, J=11.0 Hz, 1H), 2.50 (d, J=2.1 Hz, 1H), 1.98-1.68 (m, 3H), 1.63-1.48 (m, 1H), 1.36 (td, J=12.3, 8.4 Hz, 1H), 0.84-0.76 (m, 4H); LC-MS: m/z 474.1 (MH+)

The racemic mixture (Example 185c) was then separated into single enantiomers by chiral semi-preparative HPLC

| | | |
|---|---|---|
| | Column | Chiralcel OD-H (25 × 2 cm), 5μ |
| | Mobile phase | n-Hexane/(Ethanol + isopropyl |
| | | amine 50/50% v/v |
| | Flow rate (ml/min) | 17 ml/min |
| | DAD detection | 220 nm |
| Preparative chiral | Loop | 2000 μL |
| chromatography | Total amount | 42 mg |
| protocol: | Injection | 9.2 mg (each injection) |

| 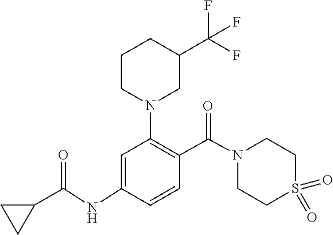 [SMILES: O=C(Nc1ccc(C(=O)N2CCS(=O)(=O)CC2)c(N2CCCC(C(F)(F)F)C2)c1)C1CC1] | Enantiomer 1 | Rt 7.15 min | 100% ee |
|---|---|---|---|
| 3R or 3S enantiomer | LC-MS: m/z 474.2 (MH+) | | |
| | N-[4-(1,1-dioxo-1,4-thiazinane-4-carbonyl)-3-[3R or 3S-(tri- | | |
| | fluoromethyl)piperidin-1-yl]phenyl]cyclopropanecarboxamide | | |
| | Example 185a | | |
| 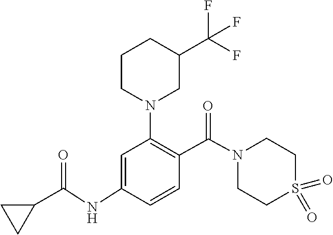 [SMILES: O=C(Nc1ccc(C(=O)N2CCS(=O)(=O)CC2)c(N2CCCC(C(F)(F)F)C2)c1)C1CC1] | Enantiomer 2 | Rt 16.45 min | 100% ee |

| | |
|---|---|
| 3R or 3S enantiomer | LC-MS: m/z 474.2 (MH+) |
| | N-[4-(1,1-dioxo-1,4-thiazinane-4-carbonyl)-3-[3R or 3S-(tri- |
| | fluoromethyl)piperidin-1-yl]phenyl]cyclopropanecarboxamide |
| | Example 185b |

Preparation: N-[4-(1,1-dioxo-1,4-thiazinane-4-carbonyl)-3-[2-(trifluoromethyl)morpholin yl]phenyl]cyclopropanecarboxamide

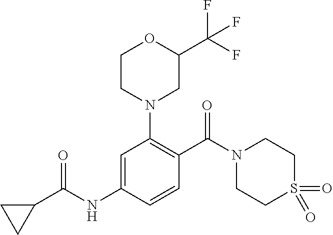 [SMILES: O=C(Nc1ccc(C(=O)N2CCS(=O)(=O)CC2)c(N2CCOC(C(F)(F)F)C2)c1)C1CC1]

### Example 186

The synthesis of the title compound was effected analogously to the synthesis of the compound of Example 183a-c (N-[4-(1,1-dioxo-1,4-thiazinane-4-carbonyl)-3-[3-(trifluoromethyl)pyrrolidin-1-yl]phenyl]cyclopropanecarboxamide) wherein [4-amino-242-(trifluoromethyl)morpholin-4-yl]phenyl]-(1,1-dioxo-1,4-thiazinan-4-yl)methanone was used instead of [4-amino-2-[3-(trifluoromethyl)pyrrolidin-1-yl]phenyl]-(1,1-dioxo-1,4-thiazinan-4-yl)methanone. The title compound was obtained in a yield of 43% (20 mg) as a racemic mixture.

1H NMR (400 MHz, DMSO-d6) δ 10.33 (d, J=8.7 Hz, 1H), 7.45 (d, J=28.3 Hz, 1H), 7.34 (d, J=10.2 Hz, 1H), 7.25 (d, J=8.2 Hz, 1H), 4.54-4.18 (m, 2H), 4.02 (dd, J=50.7, 11.3 Hz, 1H), 3.64 (dd, J=34.4, 18.7 Hz, 4H), 3.38 (d, J=11.5 Hz, 1H), 3.26-2.83 (m, 6H), 2.72-2.54 (m, 1H), 1.77 (s, 1H), 0.80 (d, J=7.5 Hz, 4H); LC-MS: 476.2 (MH+).

Preparation: N-[3-(3-azabicyclo[2.2.1]heptan-3-yl)-4-(1,1-dioxo-1,4-thiazinane-4-carbonyl)phenyl]cycloprooanecarboxamide

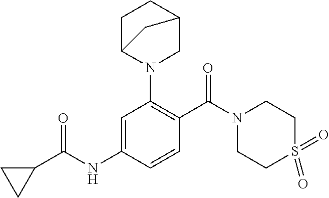 [SMILES: O=C(Nc1ccc(C(=O)N2CCS(=O)(=O)CC2)c(N2CC3CCC2C3)c1)C1CC1]

### Example187

The synthesis of the title compound was effected analogously to the synthesis of the compound of Example 183a-c (N-[4-(1,1-dioxo-1,4-thiazinane-4-carbonyl)-3-[3-(trifluoromethyl)pyrrolidin-1-yl]phenyl]cyclopropanecarboxamide) wherein [4-amino-2-(3-azabicyclo[2.2.1]heptan-3-yl)phenyl]-(1,1-dioxo-1,4-thiazinan-4-yl)methanone was used instead of [4-amino-2-[3-(trifluoromethyl)pyrrolidin-1-yl]phenyl]-(1,1-dioxo-1,4-thiazinan-4-yl)methanone. The title compound was obtained in a yield of 23% (53 mg) as a mixture of diasteroisomers.

1H NMR (500 MHz, DMSO-d6) δ 10.14-10.07 (m, 1 H), 7.14-7.04 (m, 1 H), 7.03-7.00 (m, 1 H), 6.97-6.88 (m, 1 H), 4.53-4.26 (m, 1 H), 4.01-3.83 (m, 1 H), 4.01-3.37 (m, 3 H), 3.46-2.91 (m, 5 H), 2.54 -2.44 (m, 1 H), 2.64-2.20 (m, 1 H), 1.81-1.72 (m, 1 H), 1.84-1.19 (m, 6 H), 0.87-0.70 (m, 4 H); LC-MS: m/z 418.2 (MH+).

Preparation: N-[3-[(anti)-2,6-dimethylmorpholin-4-yl]-4-(1,1-dioxo-1,4-thiazinane-4-carbonyl)phenyl]cyclopropanecarboxamide

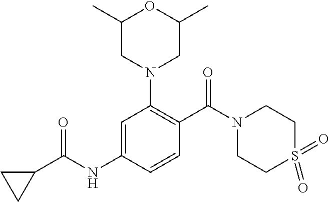 [SMILES: CC1CN(c2cc(NC(=O)C3CC3)ccc2C(=O)N2CCS(=O)(=O)CC2)CC(C)O1]

### Example 188

The synthesis of the title compound was effected analogously to the synthesis of the compound of Example 183a-c (N-[4-(1,1-dioxo-1,4-thiazinane-4-carbonyl)-3-[3-(trifluoromethyl)pyrrolidin-1-yl]phenyl]cyclopropanecarboxamide) wherein [4-amino-2-[(anti)-2,6-dimethylmorpholin-4-yl]phenyI]-(1,1-dioxo-1,4-thiazinan-4-yl)methanone was used instead of [4-amino-2-[3-(trifluoromethyl)pyrrolidin-1-yl]phenyl]-(1,1-dioxo-1,4-thiazinan-4-yl)methanone. The title compound was obtained in a yield of 32% (57 mg) as a racemic mixture.

1H NMR (400 MHz, DMSO-d6) δ 10.28 (s, 1H), 7.45-7.37 (m, 1H), 7.34-7.28 (m, 1H), 7.24 (t, J=8.9 Hz, 1H), 4.68-4.57 (m, 1H), 4.00-3.93 (m, 1H), 3.58 (d, J=15.9 Hz, 1H), 3.47 (q, J=13.8, 11.8 Hz, 2H), 3.34 (s, 2H), 3.22-2.91 (m, 4H), 2.75 (d, J=4.7 Hz, 3H), 1.76 (tt, J=6.7, 5.4 Hz, 1H), 1.14 (dd, J=11.2, 6.4 Hz, 6H), 0.83-0.74 (m, 4H); LC-MS: m/z 436.4 (MH+)

Preparation: N-[3-(5-azaspiro[2.5]octan-5-yl)-4-(1,1-dioxo-1,4-thiazinane-4-carbonyl)phenyl]cyclopropanecarboxamide

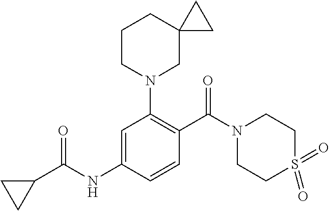 [SMILES: O=C(Nc1ccc(C(=O)N2CCS(=O)(=O)CC2)c(N2CCCC3(CC3)C2)c1)C1CC1]

### Example 189

The synthesis of the title compound was effected analogously to the synthesis of the compound of Example 183a-c (N-[4-(1,1-dioxo-1,4-thiazinane-4-carbonyl)-3-[3-(trifluoromethyl)pyrrolidin-1-yl]phenyl]cyclopropanecarboxamide) wherein [4-amino-2-(5-azaspiro[2.5]octan-5-yl)phenyI]-(1,1-dioxo-1,4-thiazinan-4-yl)methanone was used instead of [4-amino-2-[3-(trifluoromethyl)pyrrolidin-1-yl]phenyl]-(1,1-dioxo-1,4-thiazinan-4-yl)methanone. The title compound was obtained in a yield of 40% (65 mg).

1H NMR (400 MHz, DMSO-d6) δ 10.26 (s, 1H), 7.43 (d, J=1.9 Hz, 1H), 7.26 (dd, J=8.3, 1.8 Hz, 1H), 7.13 (d, J=8.2 Hz, 1H),4.21-4.05 (m, 1H), 3.96 (d, J=8.1 Hz, 1H), 3.50 (s, 2H), 3.30 (s, 1H), 3.14 (dd, J=23.5, 9.5 Hz, 5H), 2.88-2.62 (m, 2H), 1.83-1.55 (m, 3H), 1.36 (s, 2H), 0.89-0.66 (m, 4H), 0.32 (d, J=11.8 Hz, 4H); LC-MS: m/z 432.4 (MH+)

Preparation: N-[4-(1,1-dioxo-1,4-thiazinane-4-carbonyl)-3-(4-oxa-7-azaspiro[2.5]octan-7-yl)phenyl]cyclopropanecarboxamide

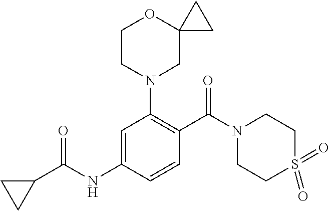 [SMILES: O=C(Nc1ccc(C(=O)N2CCS(=O)(=O)CC2)c(N2CCOC3(CC3)C2)c1)C1CC1]

### Example 190

The synthesis of the title compound was effected analogously to the synthesis of the compound of Example 183a-c (N-[4-(1,1-dioxo-1,4-thiazinane-4-carbonyl)-3-[3-(trifluoromethyl)pyrrolidin-1-yl]phenyl]cyclopropanecarboxamide) wherein [4-amino-2-(4-oxa-7-azaspiro[2.5]octan-7-yl)phenyI]-(1,1-dioxo-1,4-thiazinan-4-yl)methanone was used instead of [4-amino-2-[3-(trifluoromethyl)pyrrolidin-1-yl]phenyl]-(1,1-dioxo-1,4-thiazinan-4-yl)methanone. The title compound was obtained in a yield of 64% (19 mg).

1H NMR (400 MHz, DMSO-d6) δ 10.30 (s, 1H), 7.45 (d, J=1.9 Hz, 1H), 7.30 (dd, J=8.3, 1.8 Hz, 1H), 7.18 (d, J=8.3 Hz, 1H), 4.17 (s, 1H), 3.88 (t, J=11.3 Hz, 1H), 3.70 (q, J=4.4 Hz, 2H), 3.60-3.45 (m, 2H), 3.28 (s, 1H), 3.16-3.00 (m, 5H), 2.83-2.75 (m, 1H), 2.71 (d, J=11.5 Hz, 1H), 1.77 (p, J=6.2 Hz, 1H), 0.83-0.76 (m, 4H), 0.74-0.65 (m, 2H), 0.53 (q, J=4.5, 4.1 Hz, 2H); LC-MS: m/z 434.2 (MH+).

Preparation: N-[3-(3,3-difluoropiperidin-1-yl)-4-(1,1-dioxo-1,4-thiazinane-4-carbonyl)phenyl]cyclopropanecarboxamide

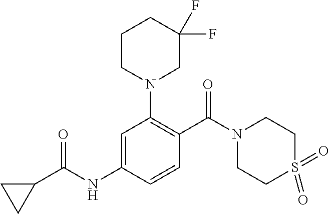 [SMILES: O=C(Nc1ccc(C(=O)N2CCS(=O)(=O)CC2)c(N2CCCC(F)(F)C2)c1)C1CC1]

### Example 191

The synthesis of the title compound was effected analogously to the synthesis of the compound of Example 183a-c (N-[4-(1,1-dioxo-1,4-thiazinane-4-carbonyl)-3-[3-(trifluoromethyl)pyrrolidin-1-yl]phenyl]cyclopropanecarboxamide) wherein [4-amino-2-(3,3-difluoropiperidin-1-yl)phenyl]-(1,1-dioxo-1,4-thiazinan-4-yl)methanone was used instead of [4-amino-2-[3-(trifluoromethyl)pyrrolidin-1-yl ]phenyl]-(1,1-dioxo-1,4-thiazinan-4-yl)methanone. The title compound was obtained in a yield of 60% (66 mg).

1H NMR (400 MHz, DMSO-d6) δ 10.33 (s, 1H), 7.52 (d, J=1.9 Hz, 1H), 7.31 (dd, J=8.3, 1.8 Hz, 1H), 7.17 (d, J=8.3 Hz, 1H), 4.12 (t, J=10.8 Hz, 1H), 3.91 (t, J=10.8 Hz, 1H), 3.47 (q, J=6.0 Hz, 2H), 3.30 (s, 2H), 3.10-2.99 (m, 5H), 2.85 (s, 1H), 2.00 (d, J=7.1 Hz, 2H), 1.77 (p, J=6.2 Hz, 3H), 0.83-0.76 (m, 4H); LC-MS: m/z 442.2 (MH+).

Preparation: N-[4-(1,1-dioxo-1,4-thiazinane-4-carbonyl)-3-(2-oxa-5-azabicyclo[2.2.1]heptan-5-yl)phenyl]cyclopropanecarboxamid

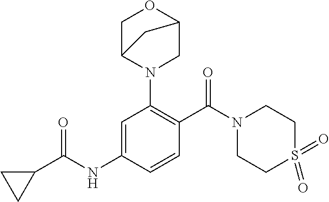 [SMILES: O=C(Nc1ccc(C(=O)N2CCS(=O)(=O)CC2)c(N2CC3CC2CO3)c1)C1CC1]

### Example 192

The synthesis of the title compound was effected analogously to the synthesis of the compound of Example 183a-c (N-[4-(1,1-dioxo-1,4-thiazinane-4-carbonyl)-3-[3-(trifluoromethyl)pyrrolidin-1-yl]phenyl]cyclopropanecarboxamide) wherein [4-amino-2-(2-oxa-5-azabicyclo[2.2.1]heptan-5-yl)phenyl]-(1,1-dioxo-1,4-thiazinan-4-yl)methanone was used instead of [4-amino-2-[3-(trifluoromethyl)pyrrolidin-1-yl]phenyl]-(1,1-dioxo-1,4-thiazinan-4-yl)methanone. The title compound was obtained in a yield of 66% (221 mg) as a mixture of diasteroisomers.

1H NMR (400 MHz, DMSO) δ 10.15 (s, 1H), 7.29-6.86 (m, 3H), 4.52 (d, J=9.9 Hz, 1H), 4.38 (s, 1H), 4.26 (s, 1H), 3.96-3.33 (m, 7H), 3.24 (s, 1H), 3.11-2.60 (m, 3H), 1.98-1.66 (m, 3H), 0.79 (dt, J=7.9, 2.3 Hz, 4H); LC-MS: m/z 420.5 (MH+).

Preparation: N-[4-(1,1-dioxo-1,4-thiazinane-4-carbonyl)-3-(6-oxa-3-azabicyclo[3.1.1]heptan-3-yl)phenyl]cyclopropanecarboxamide

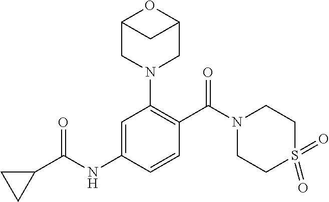 [SMILES: O=C(Nc1ccc(C(=O)N2CCS(=O)(=O)CC2)c(N2CC3CC(C2)O3)c1)C1CC1]

### Example 193

The synthesis of the title compound was effected analogously to the synthesis of the compound of Example 183a-c (N-[4-(1,1-dioxo-1,4-thiazinane-4-carbonyl)-3-[3-(trifluoromethyl)pyrrolidin-1-yl]phenyl]cyclopropanecarboxamide) wherein [4-amino-2-(6-oxa-3-azabicyclo[3.1.1]heptan-3-yl)phenyl]-(1,1-dioxo-1,4-thiazinan-4-yl)methanone was used instead of [4-amino-2-[3-(trifluoromethyl)pyrrolidin-1-yl]phenyl]-(1,1-dioxo-1,4-thiazinan-4-yl)methanone. The title compound was obtained in a yield of 12% (8 mg).

1H NMR (400 MHz, DMSO) δ 10.20 (s, 1H), 7.36 (d, J=1.8 Hz, 1H), 7.18 (d, J=8.2 Hz, 1H), 7.09 (dd, J=8.3, 1.8 Hz, 1H), 4.61 (s, 2H), 4.46 (d, J=14.5 Hz, 1H), 3.80 (d, J=14.9 Hz, 1H), 3.59 (t, J=12.1 Hz, 2H), 3.50-3.33 (m, 5H), 3.25-3.13 (m, 2H), 3.04 (q, J=7.2 Hz, 2H), 1.93 (d, J=8.4 Hz, 1H), 1.78 (p, J=7.1 Hz, 1H), 0.80 (dt, J=7.6, 2.2 Hz, 4H); LC-MS: m/z 420.47 (MH+).

Preparation: N-[4-(1,1-dioxo-1,4-thiazinane-4-carbonyl)-3-(8-oxa-3-azabicyclo[3.2.1]octan-3-yl)phenyl]cyclopropanecarboxamide

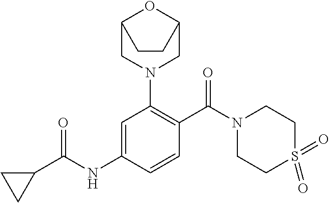 [SMILES: O=C(Nc1ccc(C(=O)N2CCS(=O)(=O)CC2)c(N2CC3CCC(C2)O3)c1)C1CC1]

### Example 194

The synthesis of the title compound was effected analogously to the synthesis of the compound of Example 183a-c (N-[4-(1,1-dioxo-1,4-thiazinane-4-carbonyl)-3-[3-(trifluoromethyl)pyrrolidin-1-yl]phenyl]cyclopropanecarboxamide) wherein [4-amino-2-(8-oxa-3-azabicyclo[3.2.1]octan-3-yl)phenyl]-(1,1-dioxo-1,4-thiazinan-4-yl)methanone was used instead of [4-amino-2-[3-(trifluoromethyl)pyrrolidin yl]phenyl]-(1,1-dioxo-1,4-thiazinan-4-yl)methanone. The title compound was obtained in a yield of 40% (88 mg).

1H NMR (400 MHz, DMSO-d6) δ 10.26 (s, 1H), 7.38 (d, J=1.8 Hz, 1H), 7.29 (dd, J=8.3, 1.8 Hz, 1H), 7.24 (d, J=8.3 Hz, 1H), 4.76-4.65 (m, 1H), 4.37 (s, 1H), 4.25 (s, 1H), 3.62 (d, J=14.9 Hz, 1H), 3.47 (d, J=11.6 Hz, 1H), 3.34 (d, J=10.9 Hz, 2H), 3.19 (dd, J=11.6, 3.7 Hz, 1H), 3.12-2.91 (m, 4H), 2.74 (d, J=10.8 Hz, 1H), 2.69-2.61 (m, 1H), 1.86-1.66 (m, 5H), 0.83-0.74 (m, 4H); LC-MS: m/z 434.2 (MH+).

Preparation: N-[3-(3-azabicyclo[3.1.1]heptan-3-yl)-4-(1,1-dioxo-1,4-thiazinane-4-carbonyl)phenyl]cyclopropanecarboxamide

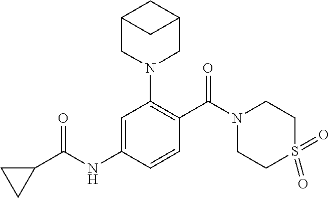 [SMILES: O=C(Nc1ccc(C(=O)N2CCS(=O)(=O)CC2)c(N2CC3CC(C3)C2)c1)C1CC1]

### Example 195

The synthesis of the title compound was effected analogously to the synthesis of the compound of Example 183a-c (N-[4-(1,1-dioxo-1,4-thiazinane-4-carbonyl)-3-[3-(trifluoromethyl)pyrrolidin-1-yl]phenyl]cyclopropanecarboxamide) wherein [4-amino-2-(3-azabicyclo[3.1.1]heptan-3-yl)phenyl]-(1,1-dioxo-1,4-thiazinan-4-yl)methanone was used instead of [4-amino-2-[3-(trifluoromethyl)pyrrolidin-1-yl]phenyl]-(1,1-dioxo-1,4-thiazinan-4-yl)methanone. The title compound was obtained in a yield of 6% (10 mg).

1H NMR (400 MHz, DMSO) δ 10.17 (s, 1H), 7.27 (d, J=1.8 Hz, 1H), 7.13 (d, J=8.3 Hz, 1H), 7.01 (dd, J=8.3, 1.7 Hz, 1H), 4.51 (d, J=14.1 Hz, 2H), 3.82 (d, J=14.9 Hz, 2H), 3.51 (dd, J=21.8, 10.7 Hz, 5H), 3.25-3.11 (m, 3H), 3.03 (d, J=13.6 Hz, 2H), 2.08 (s, 2H), 1.76 (q, J=6.1 Hz, 1H), 1.35 (dd, J=6.4, 2.8 Hz, 2H), 0.98-0.51 (m, 4H); LC-MS: m/z 418.2 (MH+).

Preparation: N-[3-(4,4-difluoropiperidin-1-yl)-4-(1,1-dioxo-1,4-thiazinane-4-carbonyl)phenyl]cyclopropanecarboxamide

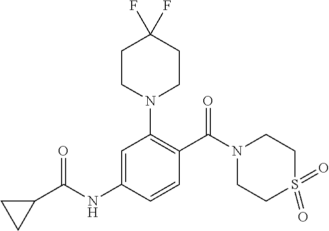 [SMILES: O=C(Nc1ccc(C(=O)N2CCS(=O)(=O)CC2)c(N2CCC(F)(F)CC2)c1)C1CC1]

### Example 196

The synthesis of the title compound was effected analogously to the synthesis of the compound of Example 183a-c (N-[4-(1,1-dioxo-1,4-thiazinane-4-carbonyl)-3-[3-(trifluoromethyl)pyrrolidin-1-yl]phenyl]cyclopropanecarboxamide) wherein [4-amino-2-(4,4-difluoropiperidin-1-yl )phenyl]-(1,1-dioxo-1,4-thiazinan-4-yl)methanone was used instead of [4-amino-2-[3-(trifluoromethyl)pyrrolidin-1-yl ]phenyl]-(1,1-dioxo-1,4-thiazinan-4-yl)methanone. The title compound was obtained in a yield of 45% (93 mg).

1H NMR (400 MHz, DMSO-d6) δ 10.30 (s, 1H), 7.50 (d, J=1.8 Hz, 1H), 7.30 (dd, J=8.3, 1.8 Hz, 1H), 7.21 (d, J=8.3 Hz, 1H), 4.31 (d, J=13.9 Hz, 1H), 3.79 (t, J=11.6 Hz, 1H), 3.56 (d, J=5.6 Hz, 2H), 3.32 (s, 1H), 3.21-3.03 (m, 4H), 3.02-2.87 (m, 3H), 2.03 (s, 4H), 1.76 (p, J=6.2 Hz, 1H), 0.83-0.74 (m, 4H); LC-MS: m/z 442.4 (MH+).

Preparation: N-[4-(1,1-dioxo-1,4-thiazinane-4-carbonyl)-3-piperidin-1-ylphenyl]cyclopropanecarboxamide

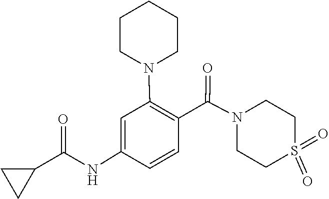 [SMILES: O=C(Nc1ccc(C(=O)N2CCS(=O)(=O)CC2)c(N2CCCCC2)c1)C1CC1]

### Example 197

The synthesis of the title compound was effected analogously to the synthesis of the compound of Example 183a-c (N-[4-(1,1-dioxo-1,4-thiazinane-4-carbonyl)-3-[3-(trifluoromethyl)pyrrolidin-1-yl]phenyl]cyclopropanecarboxamide) wherein (4-amino-2-piperidin-1-ylphenyl)-(1,1-dioxo-1,4-thiazinan-4-yl)methanone was used instead of [4-amino-2-[3-(trifluoromethyl)pyrrolidin-1-yl]phenyl]-(1,1-dioxo-1,4-thiazinan-4-yl)methanone. The title compound was obtained in a yield of 75% (135 mg).

H NMR (500 MHz, DMSO-d6) δ ppm 10.29 (s, 1 H), 7.45 (d, J=1.8 Hz, 1 H), 7.25 (dd, J=8.2, 1.8 Hz, 1 H), 7.14 (d, J=8.2 Hz, 1 H), 4.30-3.43 (m, 4 H), 3.37-3.04 (m, 4 H), 3.05-2.67 (m, 4 H), 1.81-1.73 (m, 1 H), 1.66-1.43 (m, 6 H), 0.83-0.76 (m, 4 H); LC-MS: m/z 406.2 (MH+).

Preparation: N-[4-(1,1-dioxo-1,4-thiazinane-4-carbonyl)-3-morpholin-4-ylphenyl]cyclopropanecarboxamide

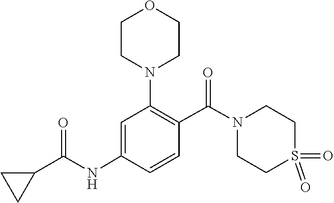 [SMILES: O=C(Nc1ccc(C(=O)N2CCS(=O)(=O)CC2)c(N2CCOCC2)c1)C1CC1]

### Example 198

The synthesis of the title compound was effected analogously to the synthesis of the compound of Example 183a-c (N-[4-(1,1-dioxo-1,4-thiazinane-4-carbonyl)-3-[3-(trifluoromethyl)pyrrolidin-1-yl]phenyl]cyclopropanecarboxamide) wherein (4-amino-2-morpholin-4-ylphenyl)-(1,1-dioxo-1,4-thiazinan-4-yl)methanone was used instead of [4-amino-2-[3-(trifluoromethyl)pyrrolidin-1-yl]phenyl]-(1,1-dioxo-1,4-thiazinan-4-yl)methanone. The title compound was obtained in a yield of 61% (45 mg).

1H NMR (500 MHz, DMSO-d6) δ ppm 10.32 (s, 1 H), 7.44 (d, J=1.8 Hz, 1 H), 7.30 (dd, J=8.2, 1.9 Hz, 1 H), 7.18 (d, J=8.2 Hz, 1 H), 3.76-3.58 (m, 4 H), 4.30-3.45 (m, 4 H), 3.37-3.07 (m, 4 H), 3.07-2.70 (m, 4 H), 1.81-1.72 (m, 1 H), 0.86-0.75 (m, 4 H); LC-MS: m/z 408.2 (MH+).

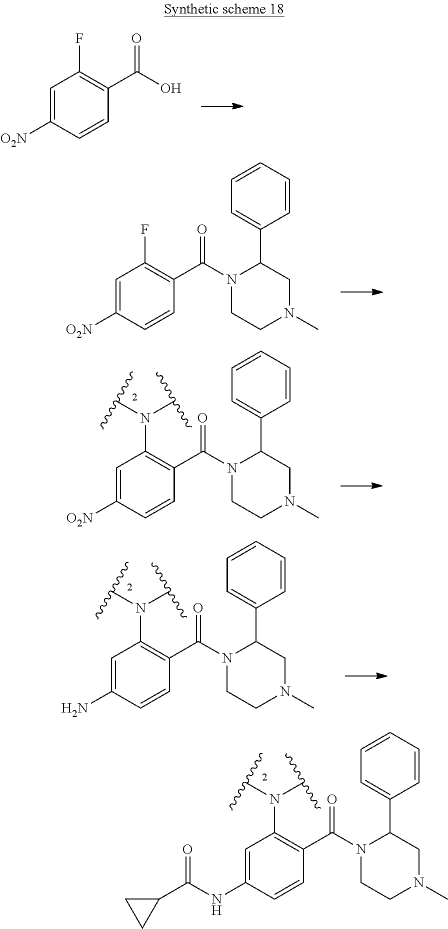

Preparation: (2-fluoro-4-nitrophenyl)-(4-methyl-2-phenylpiperazin-1-yl)methanone

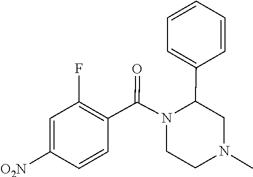 [SMILES: CN1CCN(C(=O)c2ccc([N+](=O)[O-])cc2F)C(c2ccccc2)C1]

A mixture of 2-fluoro-4-nitrobenzoic acid (2.0 g, 10.8 mmol) and thionyl dichloride (7.84 mL, 108.04 mmol) was heated to reflux for 2 h. After this time the reaction was cooled down to RT and concentrated under vacuum. The residue was dissolved in DCM (3.0 mL) and cooled to 0° C. After 10 min a solution of N,N-Diisopropylethylamine (3.76 mL, 21.61 mmol) and 1-methyl-3-phenylpiperazine (1.9 g, 10.8 mmol) in DCM (20 mL) was added. The reaction was allowed to reach the RT and stirred overnight. The day after the reaction was diluted with DCM and washed with a s.s. of NaHCO₃. The organic phase was washed with brine, dried over a phase separator and concentrated under vacuum affording the product of formula (2-fluoro-4-nitrophenyl)-(4-methyl-2-phenylpiperazin-1-yl)methanone. The product was used in the next step without further purification.
Yield: 3.46 g

1H NMR (400 MHz, DMSO-d6) δ 8.32-8.09 (m, 2 H), 7.87-7.72 (m, 1 H), 7.49 (br d, J=7.48 Hz, 1 H), 7.44 - 7.19 (m, 4 H), 3.54-3.46 (m, 1 H), 3.24-3.06 (m, 1 H), 2.97-2.86 (m, 1 H), 2.86-2.75 (m, 1 H), 2.72-2.57 (m, 1 H), 2.42-2.29 (m, 1 H), 2.26-2.12 (m, 3 H), 2.10-1.88 (m, 1 H); LC-MS: m/z 344.1 (MH+)

Preparation: (4-methyl-2-phenylpiperazin-1-yl)-[4-nitro-2-[3-(trifluoromethyl)pyrrolidin-1-yl]phenyl]methanone

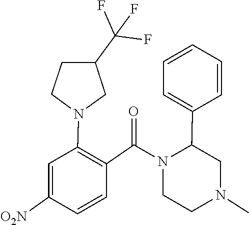 [SMILES: CN1CCN(C(=O)c2ccc([N+](=O)[O-])cc2N2CCC(C(F)(F)F)C2)C(c2ccccc2)C1]

A mixture of 3-(trifluoromethyl)pyrrolidine hydrochloride (204.55 mg, 1.16 mmol) and (2-fluoro-4-nitrophenyl)-(4-methyl-2-phenylpiperazin-1-yl)methanone (200.0 mg, 0.580 mmol) in DMSO (2.566 mL) was heated to 120° C. for 12 h. After this time the reaction was diluted with water and extracted with AcOEt (x3). Organic phase was washed 3 times with brine, dried over Na₂SO₄, filtered and concentrated under vacuum to afford the product of formula (4-methyl-2-phenylpiperazin-1-yl)-[4-nitro-2-[3-(trifluoromethyl)pyrrolidin-1-yl]phenyl]methanone as mixture of diasteroisomers. The product was used in the next step without further purification.
Yield: 214 mg

### LC:MS: m/z 463.21 (MH+).

Preparation: (4-methyl-2-phenylpiperazin-1-yl)-(2-morpholin-4-yl-4-nitrophenyl)methanone

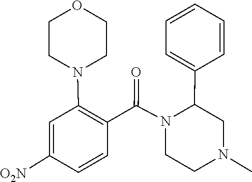 [SMILES: CN1CCN(C(=O)c2ccc([N+](=O)[O-])cc2N2CCOCC2)C(c2ccccc2)C1]

The synthesis of the title compound was effected analogously to the synthesis of (4-methyl-2-phenylpiperazin-1-yl )-[4-nitro-2-[3-(trifluoromethyl)pyrrolidin-1-yl ]phenyl]methanone wherein morpholine was used instead of 3-(trifluoromethyl)pyrrolidine hydrochloride. The title compound was obtained in a yield of 79% (0.94 g).

¹ NMR (400 MHz, DMSO-d₆) δ ppm 8.01-7.74 (m, 3 H), 7.58-7.18 (m, 5 H), 5.82-5.72 (m, 1 H), 4.52-4.40 (m, 1 H), 3.93-3.71 (m, 2 H), 3.35 (br d, J=2.86 Hz, 2 H), 3.23-3.14 (m, 1 H,) 3.07-2.60 (m, 6 H), 2.45-2.05 (m, 5 H); LC-MS: m/z 411.24 (MH+).

Preparation: (4-methyl-2-phenylpiperazin-1-yl)-(4-nitro-2-piperidin-1-ylphenyl)methanone

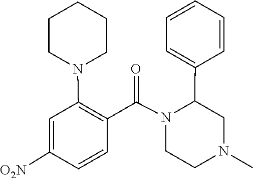 [SMILES: CN1CCN(C(=O)c2ccc([N+](=O)[O-])cc2N2CCCCC2)C(c2ccccc2)C1]

The synthesis of the title compound was effected analogously to the synthesis of (4-methyl-2-phenylpiperazin-1-yl)-[4-nitro-2-[3-(trifluoromethyl)pyrrolidin-1-yl]phenyl]methanone wherein piperidine was used instead of 3-(trifluoromethyl)pyrrolidine hydrochloride. The title compound was obtained in a yield of 92% (1.1 g).

1H NMR (400 MHz, DMSO-d6) δ ppm 7.99-7.69 (m, 3 H), 7.54-7.16 (m, 5 H), 5.83-5.71 (m, 1 H), 4.50-4.34 (m, 1 H), 3.33 (br s, 1 H), 3.28-3.12 (m, 1 H), 3.07-2.57 (m, 6 H), 2.43-2.29 (m, 1 H), 2.21 (br d, J=9.90 Hz, 3 H), 1.87-1.52 (m, 4 H), 1.41-1.23 (m, 2 H); LC-MS: m/z 409.23 (MH+).

Preparation: [2-(3,3-difluoropyrrolidin-1-yl)-4-nitrophenyI]-(4-methyl-2-phenylpiperazin-1-yl)methanone

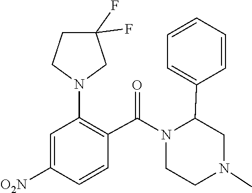 [SMILES: CN1CCN(C(=O)c2ccc([N+](=O)[O-])cc2N2CCC(F)(F)C2)C(c2ccccc2)C1]

The synthesis of the title compound was effected analogously to the synthesis of (4-methyl-2-phenylpiperazin-1-yl )-[4-nitro-2-[3-(trifluoromethyl)pyrrolidin-1-yl ]phenyl]methanone wherein 3,3-difluoropyrrolidine hydrochloride was used instead of 3-(trifluoromethyl)pyrrolidine hydrochloride. The title compound was obtained in a yield of 33% (0.415 g).

¹ NMR (400 MHz, DMSO-d₆) δ ppm 7.80-7.20 (m, 8 H), 5.86-5.69 (m, 1 H), 4.67-4.39 (m, 1 H), 3.83 (s, 3 H), 3.26-3.01 (m, 2 H), 2.57 (br s, 2 H), 2.47-1.90 (m, 7 H); LC-MS: m/z 431.24 (MH+).

Preparation: 4-amino-2-[3-(trifluoromethyl)pyrrolidin-1-yl]phenyl]-(4-methyl-2-phenylpiperazin-1-yl)methanone

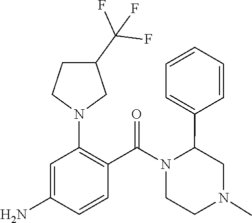 [SMILES: CN1CCN(C(=O)c2ccc(N)cc2N2CCC(C(F)(F)F)C2)C(c2ccccc2)C1]

To a solution of (4-methyl-2-phenylpiperazin-1-yl)-[4-nitro-2-[3-(trifluoromethyl)pyrrolidin-1-yl]phenyl]methanone (214.0 mg, 0.460 mmol) in Ethanol (4 mL), Pd-C 10% (98.49 mg, 0.090 mmol) was added. The reaction was stirred at RT under H2 atmosphere for 6 h. After this time, the mixture was filtered and washed with ethanol two times and concentrated under vacuum to afford the product of formula [4-amino-2-[3-(trifluoromethyl)pyrrolidin-1-yl]phenyl]-(4-methyl-2-phenylpiperazin-1-yl)methanone as mixture of diasteroisomers.
Yield: 180 mg
LC-MS: m/z 433.25 (MH+)
   Preparation: (4-amino-2-morpholin-4-ylphenyl)-(4-methyl-2-phenylpiperazin-1-yl)methanone

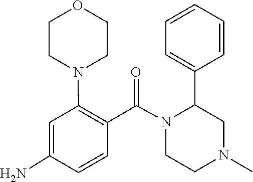 [SMILES: CN1CCN(C(=O)c2ccc(N)cc2N2CCOCC2)C(c2ccccc2)C1]

The synthesis of the title compound was effected analogously to the synthesis of the compound of Example 4-amino-2-[3-(trifluoromethyl)pyrrolidin-1-yl]phenyl]-(4-methyl-2-phenylpiperazin-1-yl)methanone wherein (4-methyl-2-phenylpiperazin-1-yl)-(2-morpholin-4-yl-4-nitrophenyl)methanone was used instead of (4-methyl-2-phenylpiperazin-1-yl)-[4-nitro-2-[3-(trifluoromethyl)pyrrolidin-1-yl]phenyl]methanone. The title compound was obtained in a yield of 99% (0.277 g).
LC-MS: m/z 381.16 (MH+)
   Preparation: (4-amino-2-piperidin-1-ylphenyI)-(4-methyl-2-phenylpiperazin-1-yl)methanone

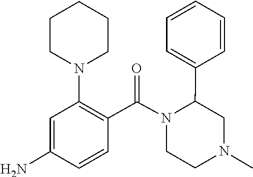 [SMILES: CN1CCN(C(=O)c2ccc(N)cc2N2CCCCC2)C(c2ccccc2)C1]

The synthesis of the title compound was effected analogously to the synthesis of the compound of Example 4-amino-2-[3-(trifluoromethyl)pyrrolidin-1-yl]phenyl]-(4-methyl-2-phenylpiperazin-1-yl)methanone wherein (4-methyl-2-phenylpiperazin-1-yl)-[4-nitro-2-piperidin-1-ylphenyl)methanone was used instead of (4-methyl-2-phenylpiperazin-1-yl)-[4-nitro-2-[3-(trifluoromethyl)pyrrolidin-1-yl]phenyl]methanone. The title compound was obtained in a yield of 91% (0.253 g).
LC-MS: m/z 380.54 (MH+)
   Preparation: [4-amino-2-(3,3-difluoropyrrolidin-1-yl )phenyl]-(4-methyl-2-phenylpiperazin-1-yl)methanone

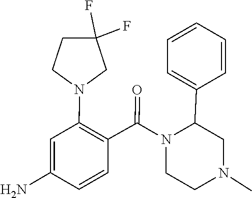 [SMILES: CN1CCN(C(=O)c2ccc(N)cc2N2CCC(F)(F)C2)C(c2ccccc2)C1]

The synthesis of the title compound was effected analogously to the synthesis of the compound of Example 4-amino-2-[3-(trifluoromethyl)pyrrolidin-1-yl]phenyl]-(4-methyl-2-phenylpiperazin-1-yl)methanone wherein [2-(3,3-difluoropyrrolidin-1-y0-4-nitrophenyl]-(4-methyl-2-phenylpiperazin-1-yl)methanone was used instead of (4-methyl-2-phenylpiperazin-1-yl )-[4-nitro-2-[3-(trifluoromethyl)pyrrolidin-1-yl ]phenyl]methanone. The title compound was obtained in a yield of 95% (0.177 g).

¹ NMR (400 MHz, DMSO-d₆) δ ppm 7.73-7.65 (m, 1 H), 7.44-7.20 (m, 5 H), 6.88-6.68 (m, 1 H), 6.16-5.97 (m, 2 H), 5.28-5.19 (m, 2 H), 3.70-3.34 (m, 6 H), 3.16-2.61 (m, 4 H), 2.48-2.41 (m, 1 H), 2.37-1.89 (m, 4 H); LC-MS: m/z 401.27 (MH+).

Preparation N-[4-(4-methyl-2-phenylpiperazine-1-carbonyl)-3-[3-(trifluoromethyl)pyrrolidin-1-yl]phenyl]cyclopropanecarboxamide

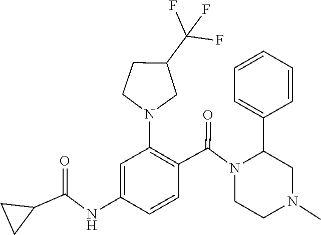 [SMILES: CN1CCN(C(=O)c2ccc(NC(=O)C3CC3)cc2N2CCC(C(F)(F)F)C2)C(c2ccccc2)C1]

### Example 199a, 199b, 199c, 199d, 199e

To a stirred solution of N,N-Diisopropylethylamine (0.14 mL, 0.830 mmol) and [4-amino-2-[3-(trifluoromethyl)pyrrolidin-1-yl]phenyl]-(4-methyl-2-phenylpiperazin-1-yl)methanone (180.0 mg, 0.420 mmol) in DCM (4.557 mL), cyclopropanecarbonyl chloride (0.05 mL, 0.540 mmol) was added dropwise and the reaction mixture was stirred at RT overnight. The day after the reaction was diluted with DCM and washed with a saturated solution of NaHCO₃ (x2). The organic phase was washed with water (X1) and then with brine, dried over a phase separator and the solvent was evaporated under reduced pressure to afford a crude, which was purified by FC on NH column (eluting from 100% of cHex to cHex/AcOEt 80:20) to afford the product of formula N-[4-(4-methyl-2-phenylpiperazine-1-carbonyl)-3-[3-(trifluoromethyl)pyrrolidin-1-yl]phenyl]cyclopropanecarboxamide as mixture of diasteroisomers.
Yield: 178 mg
LC-MS: m/z 501.24 (MH+)

The mixture of diasteroisomers was then separated into single enantiomers by chiral semi-preparative HPLC

| | | |
|---|---|---|
| | Column | Whelk O1 (R, R) (25 × 3.0 cm), 10μ |
| | Mobile phase | n-Hexane/Ethanol 75/25% v/v |
| | Flow rate (ml/min) | 37 ml/min |
| | DAD detection | 220 nm |
| Preparative chiral | Loop | 400 μL |
| chromatography | Total amount | 178 mg |
| protocol: | Injection | 9 mg (each injection) |

Diasteroisomer 1

| 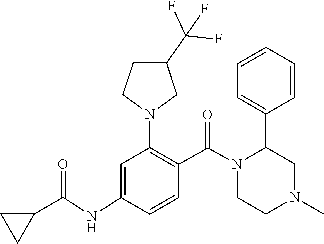 [SMILES: CN1CCN(C(=O)c2ccc(NC(=O)C3CC3)cc2N2CCC(C(F)(F)F)C2)C(c2ccccc2)C1] | Enantiomer 1, Yield: 32.3 mg | Rt 22.36 min | 100% ee |
|---|---|---|---|
| (2S, 3R) or (2R, 3S) or (2R, 3R) or (2S, 3S) | LC-MS: m/z 501.2 (MH+) | | |
| | (2S, 3R) or (2R, 3S) or (2R, 3R) or (2S, 3S) N-[4-(4-methyl- | | |
| | 2-phenylpiperazine-1-carbonyl)-3-[3-(trifluoromethyl) | | |
| | pyrrolidin-1-yl]phenyl]cyclopropanecarboxamide | | |
| | Example 199a | | |
| 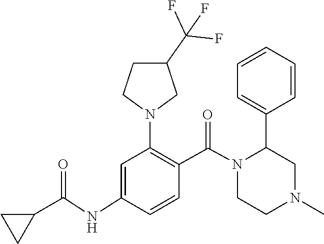 [SMILES: CN1CCN(C(=O)c2ccc(NC(=O)C3CC3)cc2N2CCC(C(F)(F)F)C2)C(c2ccccc2)C1] | Enantiomer 2, Yield: 22.9 mg | Rt 28.08 min | 100% ee |

| | |
|---|---|
| (2S, 3R) or (2R, 3S) or (2R, 3R) or (2S, 3S) | LC-MS: m/z 501.2 (MH+) |
| | (2S, 3R) or (2R, 3S) or (2R, 3R) or (2S, 3S) N-[4-(4-methyl-2- |
| | phenylpiperazine-1-carbonyl)-3-[3-(trifluoromethyl)pyrrolidin-1- |
| | yl]phenyl]cyclopropanecarboxamide |
| | Example 199b |

1H NMR (400 MHz, DMSO-d6) δ 10.16 (s, 1H), 7.67 (d, 1H), 7.45 (s, 1H), 7.42-7.22 (m, 4H), 7.17-7.01 (m, 1H),
6.96-6.76 (m, 1H), 5.84-4.27 (m, 1H), 3.60-3.44 (m, 4H), 3.05 (dd, 3H), 2.83-2.60 (m, 1H), 2.41-1.87 (m,
7H), 1.78-1.66 (m, 2H), 0.90-0.63 (m, 4H).
Diasteroisomer 2

| 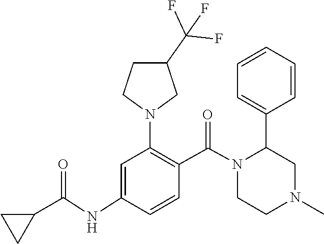 [SMILES: CN1CCN(C(=O)c2ccc(NC(=O)C3CC3)cc2N2CCC(C(F)(F)F)C2)C(c2ccccc2)C1] | Enantiomer 1, Yield: 18.8 mg | Rt 26.26 min | 100% ee |
|---|---|---|---|
| (2S, 3R) or (2R, 3S) or (2R, 3R) or (2S, 3S) | LC-MS: m/z 501.3 (MH+) | | |
| | (2S, 3R) or (2R, 3S) or (2R, 3R) or (2S, 3S) N-[4-(4-methyl-2- | | |
| | phenylpiperazine-1-carbonyl)-3-[3-(trifluoromethyl)pyrrolidin-1- | | |
| | yl]phenyl]cyclopropanecarboxamide | | |
| | Example 199c | | |
| 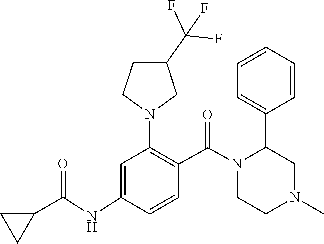 [SMILES: CN1CCN(C(=O)c2ccc(NC(=O)C3CC3)cc2N2CCC(C(F)(F)F)C2)C(c2ccccc2)C1] | Enantiomer 2, Yield: 28.3 mg | Rt 32.46 min | 100% ee |

| | |
|---|---|
| (2S, 3R) or (2R, 3S) or (2R, 3R) or (2S, 3S) | LC-MS: m/z 501.3 (MH+) |
| | (2S, 3R) or (2R, 3S) or (2R, 3R) or (2S, 3S) N-[4-(4-methyl-2- |
| | phenylpiperazine-1-carbonyl)-3-[3-(trifluoromethyl)pyrrolidin-1- |
| | yl]phenyl]cyclopropanecarboxamide |
| | Example 199d |

1H NMR (400 MHz, DMS0-d6) δ 10.16 (d, 1H), 7.57 (dd, 2H), 7.42-7.17 (m, 4H), 7.15-6.99 (m, 1H), 6.89 (d, 1H),
5.94-4.26 (m, 1H), 3.58-3.35 (m, 4H), 3.21-2.57 (m, 4H), 2.38-2.16 (m, 6H), 2.05 (s, 1H), 2.00-1.88 (m, 1H),
1.76 (s, 1H), 0.79 (d, 4H).

Preparation: (2S, 3R) or (2R, 3S) or (2R, 3R) or (2S, 3S) N-[4-(4-methyl-2-phenylpiperazine-1-carbonyl)-3-[3-(trifluoromethyl)pyrrolidin-1-yl]phenyl]cyclopropanecarboxamide hydrochloride

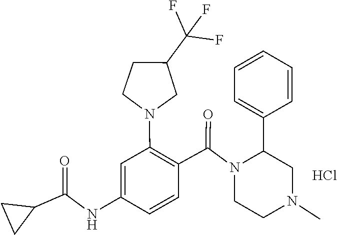 [SMILES: CN1CCN(C(=O)c2ccc(NC(=O)C3CC3)cc2N2CCC(C(F)(F)F)C2)C(c2ccccc2)C1 ; Cl]

### Example 199e

(2S, 3R) or (2R, 3S) or (2R, 3R) or (2S, 3S) N-[4-(4-methyl-2-phenylpiperazine-1-carbonyl)-3-[3-(trifluoromethyl)pyrrolidin-1-yl]phenyl]cyclopropanecarboxamide (Diasteroisomer 1 Enantiomer 1, Example 198a, 20 mg) was dissolved in MeOH (1.5 mL) and treated with 1 eq of HCI in dioxane to afford, after evaporation, the product of formula (2S, 3R) or (2R, 3S) or (2R, 3R) or (2S, 3S) N-[4-(4-methyl-2-phenylpiperazine-1-carbonyl)-3-[3-(trifluoromethyl)pyrrolidin-1-yl]phenyl]cyclopropanecarboxamide hydrochloride.
Yield: 20.8 mg
LC-MS: m/z 501.31 (MH+).
   Preparation: N-[4-(4-methyl-2-phenylpiperazine-1-carbonyl)-3-morpholin-4-ylphenyl]cyclopropanecarboxamide

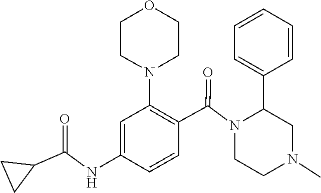 [SMILES: CN1CCN(C(=O)c2ccc(NC(=O)C3CC3)cc2N2CCOCC2)C(c2ccccc2)C1]

### Example 200

The synthesis of the title compound was effected analogously to the synthesis of the compound of Example 199a-e (N-[4-(4-methyl-2-phenylpiperazine-1-carbonyl)-3-[3-(trifluoromethyl)pyrrolidin-1-yl]phenyl]cyclopropanecarboxamide) wherein (4-amino-2-morpholin-4-ylphenyI)-(4-methyl-2-phenylpiperazin-1-yl )methanone was used instead of [4-amino-2-[3-(trifluoromethyl)pyrrolidin-1-yl]phenyl]-(4-methyl-2-phenylpiperazin-1-yl )methanone. The title compound was obtained in a yield of 64% (0.209 g).

¹ NMR (400 MHz, DMSO-d₆) δ ppm 10.34-10.20 (m, 1 H), 7.79-7.71 (m, 1 H), 7.51-6.99 (m, 7 H), 5.82-5.72 (m, 1 H), 4.55-4.35 (m, 1 H), 3.93-3.44 (m, 3 H), 3.29-3.13 (m, 3 H), 3.10 -2.99 (m, 1 H), 2.95-2.55 (m, 5 H), 2.40-2.16 (m, 3 H), 2.12-1.95 (m, 1 H), 1.86-1.69 (m, 1 H), 0.85-0.73 (m, 4 H); LC-MS: m/z 450.01 (MH+).

Preparation: N-[4-(4-methyl-2-phenylpiperazine-1-carbonyl)-3-piperidin-1-ylphenyl]cyclopropanecarboxamide

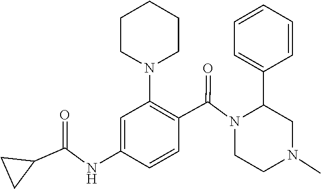 [SMILES: CN1CCN(C(=O)c2ccc(NC(=O)C3CC3)cc2N2CCCCC2)C(c2ccccc2)C1]

### Example 201a, 201b, 201c

The synthesis of the title compound was effected analogously to the synthesis of the compound of Example 199a-e (N-[4-(4-methyl-2-phenylpiperazine-1-carbonyl)-3-[3-(trifluoromethyl)pyrrolidin-1-yl]phenyl]cyclopropanecarboxamide) wherein (4-amino-2-piperidin-1-yl phenyI)-(4-methyl-2-phenylpiperazin-1-yl )methanone was used instead of [4-amino-2-[3-(trifluoromethyl)pyrrolidin-1-yl]phenyl]-(4-methyl-2-phenylpiperazin-1-yl)methanone. The title compound was obtained in a yield of 23.4% (0.072 g).

¹ NMR (400 MHz, DMSO-d₆) δ ppm 10.28 -10.15 (m, 1 H), 7.78-6.98 (m, 8 H), 5.83-4.37 (m, 2 H), 3.51-3.33 (m, 1 H), 3.27 (s, 1 H), 3.19-2.98 (m, 2 H), 2.95-2.55 (m, 4 H), 2.41-1.97 (m, 5 H), 1.84 -1.51 (m, 4 H), 1.36 (br s, 2 H), 0.83-0.73 (m, 4 H); LC-MS: m/z 447.28 (MH+).

The racemic mixture (Example 201c) was then separated into single enantiomers by chiral semi-preparative HPLC

| | Column | Chiralpak IC (25 × 2.0 cm), 5μ | |
|---|---|---|---|
| | Mobile phase | n-Hexane/(Ethanol + 0.1% | |
| | | isopropylamine) 70/30% v/v | |
| | Flow rate (ml/min) | 17 ml/min | |
| | DAD detection | 220 nm | |
| Preparative chiral | Loop | 650 μL | |
| chromatography | Total amount | 69 mg | |
| protocol: | Injection | 9 mg (each injection) | |
| 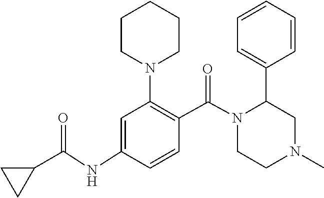 [SMILES: CN1CCN(C(=O)c2ccc(NC(=O)C3CC3)cc2N2CCCCC2)C(c2ccccc2)C1] | Enantiomer 1, Yield: 25.2 mg | Rt 6.020 min | 100% ee |
| (2R or 2S) | LC-MS: m/z 447.28 (MH+) | | |
| | N-[4-(4-methyl-(2R or 2S)-phenylpiperazine-1-carbonyl)- | | |
| | 3-piperidin-1-ylphenyl]cyclopropanecarboxamide | | |
| | Example 201a | | |
| 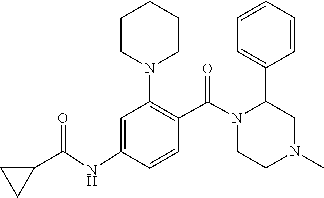 [SMILES: CN1CCN(C(=O)c2ccc(NC(=O)C3CC3)cc2N2CCCCC2)C(c2ccccc2)C1] | Enantiomer 2, Yield: 26.2 mg | Rt 8.169 min | 100% ee |

| | |
|---|---|
| (2R or 2S) | LC-MS: m/z 447.28 (MH+) |
| | N-[4-(4-methyl-(2R or 2S)-phenylpiperazine-1-carbonyl)- |
| | 3-piperidin-1-ylphenyl]cyclopropanecarboxamide |
| | Example 201b |

Preparation: N-[3-(3,3-difluoropyrrolidin-1-yl)-4-(4-methyl-2-phenylpiperazine-1-carbonyl)phenyl]cyclopropanecarboxamide

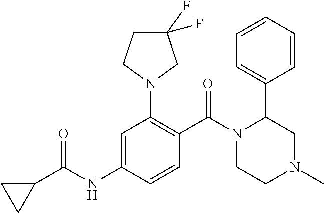 [SMILES: CN1CCN(C(=O)c2ccc(NC(=O)C3CC3)cc2N2CCC(F)(F)C2)C(c2ccccc2)C1]

### Example 202a, 202b, 202c

The synthesis of the title compound was effected analogously to the synthesis of the compound of Example 199a-e (N-[4-(4-methyl-2-phenylpiperazine-1-carbonyl)-3-[3-(trifluoromethyl)pyrrolidin-1-yl]phenyl]cyclopropanecarboxamide) wherein [4-amino-2-(3,3-difluoropyrrolidin-1-yl)phenyI]-(4-methyl-2-phenylpiperazin-1-yl)methanone was used instead of [4-amino-2-[3-(trifluoromethyl)pyrrolidin-1-yl]phenyl]-(4-methyl-2-phenylpiperazin-1-yl)methanone. The title compound was obtained in a yield of 80.5% (0.167 g).

¹ NMR (400 MHz, DMSO-d₆) δ ppm 10.29-10.15 (m, 1 H), 7.76-6.91 (m, 8 H), 5.82-4.34 (m, 1 H), 3.30 (s, 3 H), 2.41-1.66 (m, 13 H), 0.91-0.66 (m, 4 H); LC-MS: m/z 469.3 (MH+).

The racemic mixture (Example 202c) was then separated into single enantiomers by chiral semi-preparative HPLC

| | Column | Chiralpak AD-H (25 × 3.0 cm), 5μ | |
|---|---|---|---|
| | Mobile phase | n-Hexane/(Ethanol + 0.1% | |
| | | isopropylamine) 70/30% v/v | |
| | Flow rate (ml/min) | 17 ml/min | |
| | DAD detection | 220 nm | |
| Preparative chiral | Loop | 1000 μL | |
| chromatography | Total amount | 163.5 mg | |
| protocol: | Injection | 23.3 mg (each injection) | |
| 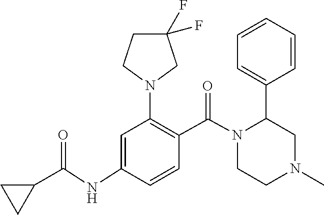 [SMILES: CN1CCN(C(=O)c2ccc(NC(=O)C3CC3)cc2N2CCC(F)(F)C2)C(c2ccccc2)C1] | Enantiomer 1, Yield: 76.1 mg | Rt 7.463 min | 100% ee |

| (2R or2S) | LC-MS: m/z 469.3 (MH+) | | |
|---|---|---|---|
| | N-[3-(3,3-difluoropyrrolidin-1-yl)-4-(2R or 2S)-4-methyl-2- | | |
| | phenylpiperazine-1-carbonyl)phenyl]cyclopropanecarboxamide | | |
| | Example 202a | | |
| 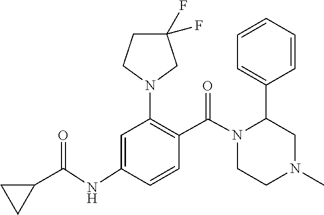 [SMILES: CN1CCN(C(=O)c2ccc(NC(=O)C3CC3)cc2N2CCC(F)(F)C2)C(c2ccccc2)C1] | Enantiomer 2, Yield: 64.8 mg | Rt 14.278 min | 100% ee |

| | |
|---|---|
| (2R or 2S) | LC-MS: m/z 469.3 (MH+) |
| | N-[3-(3,3-difluoropyrrolidin-1-yl)-4-(2R or 2S)-4-methyl-2- |
| | phenylpiperazine-1-carbonyl)phenyl]cyclopropanecarboxamide |
| | Example 202b |

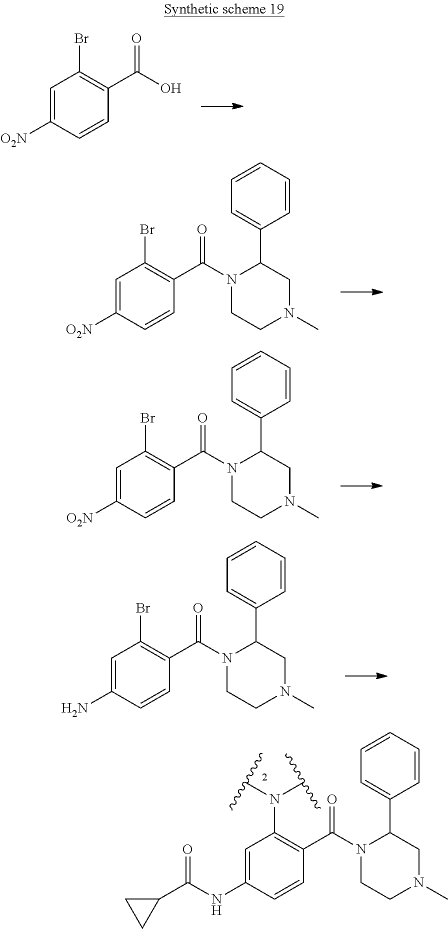

Preparation: (2-bromo-4-nitrophenyl)-(4-methyl-2-phenylpiperazin-1-yl)methanone

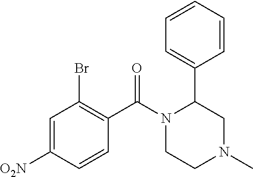 [SMILES: CN1CCN(C(=O)c2ccc([N+](=O)[O-])cc2Br)C(c2ccccc2)C1]

A mixture of 2-bromo-4-nitrobenzoic acid (1.05 g, 4.27 mmol) and thionyl dichloride (6.23 mL, 85.36 mmol) was heated to reflux for 1.30 h. After this time the reaction was cooled down to RT and concentrated under vacuum. The residue was dissolved in DCM (3 mL) and cooled to 0° C. After 10 min a solution of N,N-Diisopropylethylamine (1.78 mL, 12.8 mmol) and 1-methyl-3-phenylpiperazine (0.75 g, 4.27 mmol) in DCM (20 mL) was added. The reaction was allowed to reach the RT and stirred overnight. The day after the reaction was diluted with DCM and washed with a s.s. of NaHCO₃. The organic phase was washed with brine, dried over a phase separator and concentrated under vacuum affording the product of formula (2-bromo-4-nitrophenyl)-(4-methyl-2-phenylpiperazin-1-yl)methanone. The product was used in the next step without further purification.
Yield: 1.77 g

H NMR (400 MHz, DMSO-d₆) δ ppm 7.38 (s, 8 H), 5.83-4.36 (m, 2 H), 3.56-3.33 (m, 1 H), 3.13-2.95 (m, 2 H), 2.87-2.58 (m, 1 H), 2.45-2.34 (m, 1 H), 2.29-1.92 (m, 3 H); LC-MS: m/z 405.96, 407.8 (MH+).

Preparation: (4-amino-2-bromophenyl)-(4-methyl-2-phenylpiperazin-1-yl)methanone

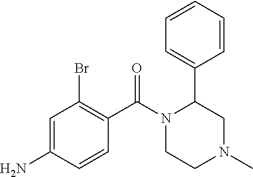 [SMILES: CN1CCN(C(=O)c2ccc(N)cc2Br)C(c2ccccc2)C1]

To a solution of (2-bromo-4-nitrophenyl)-(4-methyl-2-phenylpiperazin-1-yl)methanone (1.0 g, 2.47 mmol) in acetic acid (40 mL), zinc (1.62 g, 24.74 mmol) was added. The reaction mixture was stirred at room temperature. After 2h the reaction was filtered on hirsch funnel, washed with EtOAc and concentrated under vacuum. The crude was dissolved in 2.5 ml of MeOH and submitted to a SCX cartridge (20g, washed with MeOH and then eluted with NH₃ 1N in MeOH). The solvent was evaporated under reduced pressure and the product was purified by FC on silica gel (eluting from 100% of cHex to cHex/AcOEt 20:80), affording the product of formula (4-amino-2-bromophenyl)-(4-methyl-2-phenylpiperazin-1-yl)methanone.
Yield: 0.850 g

¹ NMR (400 MHz, DMSO-d₆) δ ppm 7.34 (br s, 8 H), 5.81-5.49 (m, 3 H), 3.54-3.35 (m, 1 H), 3.23 -2.55 (m, 3 H), 2.40-2.01 (m, 4 H), 1.89 (s, 1 H); LC-MS: m/z 374.09, 376.1 (MH+).

Preparation: N-[3-bromo-4-(4-methyl-2-phenylpiperazine-1-carbonyl)phenyl]cyclopropanecarboxamide

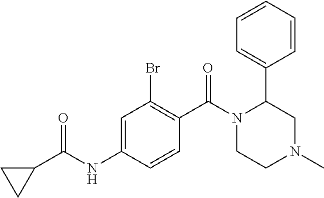 [SMILES: CN1CCN(C(=O)c2ccc(NC(=O)C3CC3)cc2Br)C(c2ccccc2)C1]

To a stirred solution of (4-amino-2-bromophenyl)-(4-methyl-2-phenylpiperazin-1-yl)methanone (0.85 g, 2.27 mmol) and N,N-Diisopropylethylamine (0.78 mL, 4.54 mmol) in DCM (20 mL) at 0 C, cyclopropanecarbonyl chloride (0.27 mL, 2.95 mmol) was added dropwise and the reaction mixture was stirred at room temperature overnight. After this time the mixture was diluted with a s.s. of NaHCO₃ and extracted with DCM (X3). The organic phase was washed with brine, dried over a phase separator and concentrated under vacuum. The crude was purified by a chromatographic column sfar Amino D 28g (gradient from cHex/AcOEt 80:20 to cHex/AcOEt 10:90), affording the product of formula N-[3-bromo-4-(4-methyl-2-phenylpiperazine-1-carbonyl)phenyl]cyclopropanecarboxamide.
Yield: 0.850 g

¹ NMR (400 MHz, DMSO-d₆)δ ppm 10.57-10.37 (m, 1 H), 8.14-7.20 (m, 8 H), 5.84-5.72 (m, 1 H), 3.71-3.41 (m, 3 H), 3.20-2.57 (m, 3 H), 2.40-1.70 (m, 4 H), 0.84 (br s, 4 H); LC-MS: m/z 442.11, 444.1 (MH+)

Preparation: N-[4-(4-methyl-2-phenylpiperazine-1-carbonyl)-3-(2-oxa-7-azaspiro[3.4]octan-7-yl)phenyl]cyclopropanecarboxamide

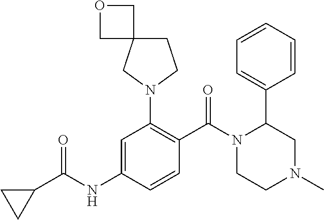 [SMILES: CN1CCN(C(=O)c2ccc(NC(=O)C3CC3)cc2N2CCC3(COC3)C2)C(c2ccccc2)C1]

### Example 203

In a 5 mL microwave vial, N-[3-bromo-4-(4-methyl-2-phenylpiperazine-1-carbonyl)phenyl]cyclopropanecarboxamide (100.0 mg, 0.230 mmol), sodium tert-butoxide (32.59 mg, 0.340 mmol), 2-oxa-6-azaspiro[3.4]octane (28.42 uL, 0.270 mmol) and dicyclohexyl-[2-[2,6-di(propan-2-yloxy)phenyl]phenyl]phosphine (26.37 mg, 0.060 mmol) and NMP (1.5 mL) were added. The resulting suspension was degased by N₂-vacuum cycles using shlenk line technique. The reaction mixture was shaken for 4h at PLS at 120° C. The crude was dissolved in 0.5 ml of MeOH and submitted to a SCX cartridge (10g, eluted with MeOH and then washed with NH3 1N in MeOH). The solvent was evaporated under reduced pressure to afford a crude that was purified by a chromatographic Sfar NH 11g (gradient from cHex/AcOEt 70:30 to AcOEt 100%) affording the product of formula N-[4-(4-methyl-2-phenylpiperazine-1-carbonyl)-3-(2-oxa-7-azaspiro[3.4]octan-7-yl)phenyl]cyclopropanecarboxamide.
Yield: 0.016 g
   ¹ NMR (400 MHz, DMSO-d₆) δ ppm 10.22-10.06 (m, 1 H), 7.83-6.84 (m, 8 H), 5.85-5.67 (m, 1 H), 4.71-4.21 (m, 6 H), 3.57-3.37 (m, 4 H), 3.17-2.58 (m, 4 H), 2.23 (s, 3 H), 2.03-1.69 (m, 3 H), 0.78 (br d, J=4.18 Hz, 4 H); LC-MS: m/z 475.49 (MH+).
      Preparation: N-[4-(4-methyl-2-phenylpiperazine-1-carbonyl)-3-(2-oxa-6-azaspiro[3.3]heptan-6-yl)phenyl]cyclopropanecarboxamide

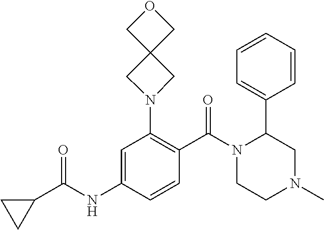 [SMILES: CN1CCN(C(=O)c2ccc(NC(=O)C3CC3)cc2N2CC3(COC3)C2)C(c2ccccc2)C1]

### Example 204

The synthesis of the title compound was effected analogously to the synthesis of the compound of Example 203 (N-[4-(4-methyl-2-phenylpiperazine-1-carbonyl)-3-(2-oxa-7-azaspiro[3.4]octan-7-yl)phenyl]cyclopropanecarboxamide), wherein 2-oxa-6-azaspiro[3.3]heptane was used instead of 2-oxa-6-azaspiro[3.4]octane. The title compound was obtained in a yield of 24% (0.025 g).

1H NMR (400 MHz, DMSO) δ 10.15 (s, 1H), 7.79 (d, J=8.0 Hz, 1H), 7.51-7.11 (m, 5H), 6.89 (dd, J=45.0, 23.6 Hz, 3H), 5.72 (s, 1H), 4.74 (s, 3H), 4.57-4.28 (m, 2H), 4.01 (d, J=13.2 Hz, 2H), 3.54 (dd, J=102.7, 8.0 Hz, 3H), 2.68 (s, 1H), 2.22 (d, J=7.9 Hz, 5H), 1.76 (s, 1H), 0.79 (s, 4H); LC-MS: m/z 461.26 (MH+).

Preparation: N-[4-(4-methyl-2-phenylpiperazine-1-carbonyl)-3-(6-oxa-2-azaspiro[3.4]octan-2-yl)phenyl]cyclopropanecarboxamide

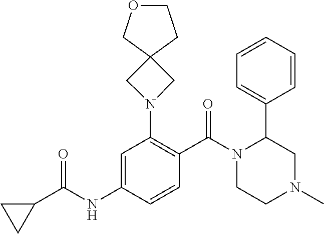 [SMILES: CN1CCN(C(=O)c2ccc(NC(=O)C3CC3)cc2N2CC3(CCOC3)C2)C(c2ccccc2)C1]

### Example 205

The synthesis of the title compound was effected analogously to the synthesis of the compound of Example 203 (N-[4-(4-methyl-2-phenylpiperazine-1-carbonyl)-3-(2-oxa-7-azaspiro[3.4]octan-7-yl)phenyl]cyclopropanecarboxamide), wherein 6-oxa-2-azaspiro[3.4]octane was used instead of 2-oxa-6-azaspiro[3.4]octane. The title compound was obtained in a yield of 7% (0.004 g).

1H NMR (500 MHz, DMSO-d6) δ 10.28-10.04 (m, 1 H), 7.82-7.11 (m, 5 H), 7.11-6.70 (m, 3 H), 5.84-4.52 (m, 1 H), 2.46-2.25 (m, 1 H), 2.24-2.17 (m, 3 H), 2.00-1.83 (m, 2 H), 4.55-1.81 (m, 13 H), 1.83-1.64 (m, 1 H), 0.85-0.67 (m, 4 H); LC-MS: m/z 476.3 (MH+).

Preparation: N-[4-(4-methyl-2-phenylpiperazine-1-carbonyl)-3-[3-(trifluoromethyl)pyrazol-1-yl]phenyl]cyclopropanecarboxamide

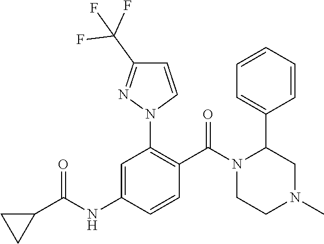 [SMILES: CN1CCN(C(=O)c2ccc(NC(=O)C3CC3)cc2-n2ccc(C(F)(F)F)n2)C(c2ccccc2)C1]

### Example 206

In a 5mL microwave vial were sequentially added N-[3-bromo-4-(4-methyl-2-phenylpiperazine-1-carbonyl)phenyl]cyclopropanecarboxamide (200.0 mg, 0.450 mmol), tripotassium;phosphate (204.41 mg, 0.950 mmol), 3-(trifluoromethyl)pyrazole (61.53 mg, 0.450 mmol) and dry 1,4-Dioxane (0.507 mL). The resulting suspension was degassed bubbling N2 for 15 minutes, then copper (I) iodide (4.33 mg, 0.020 mmol) and N,N′-Dimethylcyclohexane-1,2-diamine (6.43 mg, 0.050 mmol) were added under nitrogen flux and degassed again with Schlenk line technique. The vial was sealed and stirred overnight at 120° C. The day after the reaction mixture was diluted with EtOAc and filtered through a pad of celite. The organic layer was evaporated under reduced pressure and purified by FC Sfar C18 (eluent: water/NH4OH 0.1% 100% to water/NH4OH 0.1%/MeCN 60:40) affording the product of formula N-[4-(4-methyl-2-phenylpiperazine-1-carbonyl)-3-[3-(trifluoromethyl)pyrazol-1-yl]phenyl]cyclopropanecarboxamide.
Yield: 0.058 g

1H NMR (500 MHz, DMSO-d6) δ ppm 10.32-10.88 (m, 1 H), 7.87-8.37 (m, 2 H), 6.56-7.82 (m, 8 H), 4.43-5.74 (m, 1 H), 3.07-4.34 (m, 2 H), 2.76-3.05 (m, 1 H), 2.39-2.78 (m, 1 H), 1.33-2.38 (m, 6 H), 0.72-0.95 (m, 4 H); LC-MS: m/z 498.20 (MH+).

Preparation: N-[4-(4-methyl-2-phenylpiperazine-1-carbonyl)-3-[4-(trifluoromethyl)pyrazol-1-yl]phenyl]cyclopropanecarboxamide

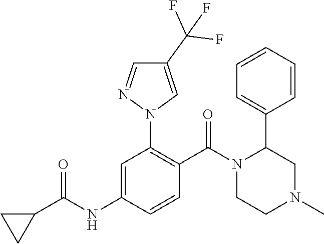 [SMILES: CN1CCN(C(=O)c2ccc(NC(=O)C3CC3)cc2-n2cc(C(F)(F)F)cn2)C(c2ccccc2)C1]

### Example 207

The synthesis of the title compound was effected analogously to the synthesis of the compound of Example 206 (N-[4-(4-methyl-2-phenylpiperazine-1-carbonyl)-3-[3-(trifluoromethyl)pyrazol-1-yl]phenyl]cyclopropanecarboxamide), wherein 4-(trifluoromethyl)-1H-pyrazole was used instead 3-(trifluoromethyl)pyrazole. The title compound was obtained in a yield of 10% (0.023 g).

1H NMR (400 MHz, DMSO-d6) δ 10.60 (d, 1H), 8.69 (d, 1H), 8.33-7.91 (m, 2H), 7.76-7.12 (m, 7H), 5.89-4.10 (m, 1H), 3.42 (d, 1H), 3.17-2.60 (m, 4H), 2.41-1.58 (m, 5H), 0.84 (d, 4H); LC-MS: m/z 498.19 (MH+).

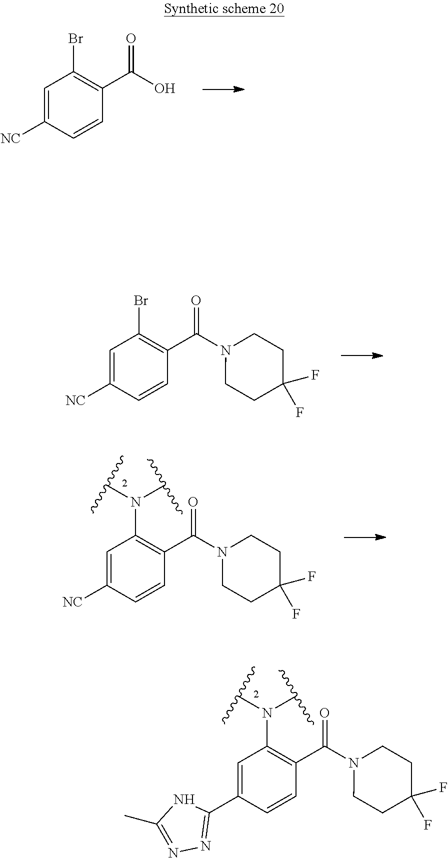 [SMILES: N#Cc1ccc(C(=O)O)c(Br)c1 ; N#Cc1ccc(C(=O)N2CCC(F)(F)CC2)c(Br)c1 ; *N(*)c1cc(C#N)ccc1C(=O)N1CCC(F)(F)CC1 ; *N(*)c1cc(-c2nnc(C)[nH]2)ccc1C(=O)N1CCC(F)(F)CC1]

Preparation: 3-bromo-4-(4,4-difluoropiperidine-1-carbonyl)benzonitrile

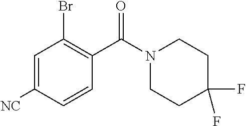

To a solution of [dimethylamino(3-triazolo[4,5-b]pyridinyloxy)methylidene]-dimethylammonium hexafluorophosphate (841.11 mg, 2.21 mmol),2-bromo-4-cyanobenzoic acid (0.5 g, 2.21 mmol) and 4,4-difluoropiperidine (267.95 mg, 2.21 mmol) in DMF (10 mL) N,N-Diisopropylethylamine (1.18 mL, 6.64 mmol) was added. The reaction was stirred at RT for 5 h. After this time the reaction was diluted with H2O. The mixture was then extracted with AcOEt 3 times. The combined organic fractions were washed with brine, dried over Na2SO4, filtered and concentrated under vacuum. The residue was purified by FC on silica gel (eluting from cHex to AcOEt/cHex 1:1) affording the product of formula 3-bromo-4-(4,4-difluoropiperidine-1-carbonyl)benzonitrile.
Yield: 0.530 g

1H NMR (400 MHz, DMSO-d6) δ 8.30 (d, 1H), 7.97 (dd, 1H), 7.66 (d, 1H), 3.86 (dt, 1H), 3.66 (ddd, 1H), 3.22 (t, 2H), 2.24-1.80 (m, 4H); LC-MS: m/z 329.06, 331.09 (MH+)

Preparation: 4-(4,4-difluoropiperidine-1-carbonyl)-3-[3-(trifluoromethyl)pyrazol-1-yl]benzonitrile

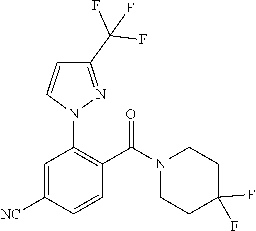 [SMILES: N#Cc1ccc(C(=O)N2CCC(F)(F)CC2)c(-n2ccc(C(F)(F)F)n2)c1]

In a vial 3-bromo-4-(4,4-difluoropiperidine-1-carbonyl)benzonitrile (150.0 mg, 0.460 mmol), tripotassium phosphate (206.04 mg, 0.960 mmol), 3-(trifluoromethyl)pyrazole (74.42 mg, 0.550 mmol) were suspended in dry 1,4-Dioxane (2.492 mL). The resulting suspension was degassed bubbling N2 for 15 minutes, then copper (I) iodide (4.36 mg, 0.020 mmol) and N,N′-Dimethylcyclohexane-1,2-diamine (6.48 mg, 0.050 mmol) were added under nitrogen flux and degassed again with Schlenk line technique. The vial was sealed and stirred ON at 120° C. The day after the reaction mixture was diluted with EtOAc and filtered through a pad of celite. The organic layer was evaporated under reduced pressure and the residue was purified by FC on NH column (eluting from 100% of cHex to cHex/AcOEt 3:7) affording the product of formula 4-(4,4-difluoropiperidine-1-carbonyl)-3-[3-(trifluoromethyl)pyrazol-1-yl]benzonitrile.
Yield: 122 mg

1H NMR (400 MHz, DMSO-d6) δ 8.59 (dd, 1H), 8.39 (d, 1H), 8.06 (dd, 1H), 7.74 (d, 1H), 7.09 (d, 1H), 3.88 (d, 1H), 3.48-3.41 (m, 1H), 3.35 (d, 2H), 2.17-1.84 (m, 4H); LC-MS: m/z 385.2 (MH+).

Preparation: 3-(3-cyclopropylpyrazol-1-yl)-4-(4,4-difluoropiperidine-1-carbonyl)benzonitrile

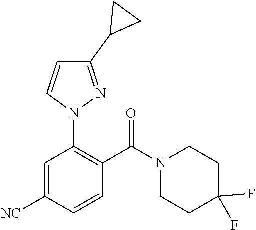 [SMILES: N#Cc1ccc(C(=O)N2CCC(F)(F)CC2)c(-n2ccc(C3CC3)n2)c1]

The synthesis of the title compound was effected analogously to the synthesis of 4-(4,4-difluoropiperidine-1-carbonyl)-3-[3-(trifluoromethyl)pyrazol-1-yl]benzonitrile wherein 3-cyclopropyl-1H-pyrazole was used instead of 3-(trifluoromethyl)pyrazole. The title compound was obtained in a yield of 44% (96 mg).

1H NMR (400 MHz, DMSO) δ 8.21 (dd, J=5.3, 2.0 Hz, 2H), 7.85 (dd, J=7.9, 1.5 Hz, 1H), 7.61 (d, J=7.9 Hz, 1H), 6.30 (d, J=2.5 Hz, 1H), 3.73 (dt, J=12.3, 5.7 Hz, 1H), 3.63 (ddd, J=13.1, 7.8, 4.5 Hz, 1H), 3.29-3.22 (m, 1H), 3.19-3.08 (m, 1H), 2.21-1.68 (m, 5H), 0.98-0.86 (m, 2H), 0.70 (ddd, J=9.5, 5.0, 3.2 Hz, 1H), 0.62 (ddd, J=9.3, 6.3, 3.2 Hz, 1H); LC-MS: m/z 357.3 (MH+).

Preparation: 4-(4,4-difluoropiperidine-1-carbonyl)-3-(3-propan-2-ylpyrazol-1-yl)benzonitrile

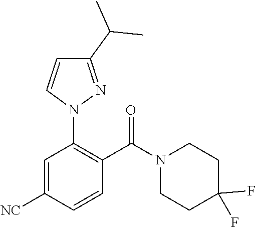 [SMILES: CC(C)c1ccn(-c2cc(C#N)ccc2C(=O)N2CCC(F)(F)CC2)n1]

The synthesis of the title compound was effected analogously to the synthesis of 4-(4,4-difluoropiperidine-1-carbonyl)-3-[3-(trifluoromethyl)pyrazol-1-yl]benzonitrile wherein 3-propan-2-yl-1H-pyrazole was used instead of 3-(trifluoromethyl)pyrazole. The title compound was obtained in a yield of 28% (62 mg).

1H NMR (400 MHz, DMSO) δ 8.22 (dd, J=7.7, 2.0 Hz, 2H), 7.86 (dd, J=7.9, 1.5 Hz, 1H), 7.63 (d, J=7.9 Hz, 1H), 6.44 (d, J=2.5 Hz, 1H), 3.77-3.58 (m, 2H), 3.28-3.23 (m, 1H), 3.11 (ddd, J=13.4, 7.7, 4.4 Hz, 1H), 2.89 (p, J=7.0 Hz, 1H), 2.18-1.65 (m, 4H), 1.26-1.13 (m, 6H); LC-MS: m/z 359.2 (MH+).

Preparation: (4,4-difluoropiperidin-1-yl)-[4-(5-methyl-4H-1,2,4-triazol-3-yl)-2-[3-(trifluoromethylpyrazol-1-yl]phenyl]methanone

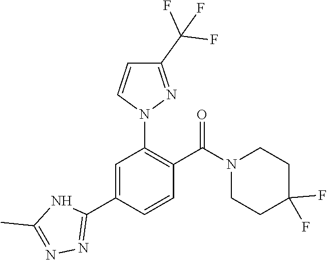 [SMILES: Cc1nnc(-c2ccc(C(=O)N3CCC(F)(F)CC3)c(-n3ccc(C(F)(F)F)n3)c2)[nH]1]

### Example 208

A mixture of 4-(4,4-difluoropiperidine-1-carbonyl)-3-[3-(trifluoromethyl)pyrazol-1-yl]benzonitrile (123.19 mg, 0.320 mmol), dicesium carbonate (313.32 mg, 0.960 mmol), ethanimidamide hydrochloride (45.46 mg, 0.480 mmol) and bromocopper (5.0 mg, 0.030 mmol) in DMSO (3.429 mL) was stirred at 120° C. for 3h. After this time the reaction was cooled down to RT and H2O 2O was added. The mixture was then extracted with AcOEt 3 times. The combined organic fractions were washed with Brine, dried over Na2SO4, filtered and concentrated under vacuum. The residue was purified by FC on NH column (from DCM to DCM/MeOH 9:1) and again by FC on RP e using acid conditions (eluting from 5:95 of CH3CN/H2O +0.1% of FA to 30:70 of CH3CN/H2O+1% of FA) affording product of formula (4,4-difluoropiperidin-1-yl)-( yl)-[4-5-methyl-4H-1,2,4-triazol-3-yl)-2-[3-(trifluoromethyl)pyrazol-1-yl]phenyl]methanone.
Yield: 42 mg

1H NMR (400 MHz, DMSO-d6) δ 13.90 (s, 1H), 8.57 (dd, 1H), 8.21 (s, 1H), 8.16-8.12 (m, 1H), 7.60 (s, 1H), 7.04 (d, 1H), 3.94 (d, 1H), 3.38 (d, 3H), 2.44 (s, 3H), 2.09 (s, 1H), 1.96 (dd, 3H); LC-MS: m/z 441.2 (MH+).

Preparation: [2-(3-cyclopropylpyrazol-1-yl)-4-(5-methyl-4H-1,2,4-triazol-3-yl)phenyl]-(4,4-difluoropiperidin-1-yl)methanone

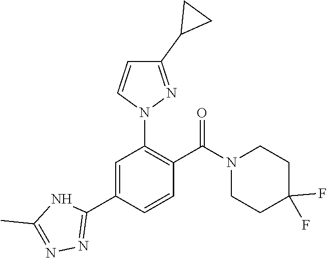 [SMILES: Cc1nnc(-c2ccc(C(=O)N3CCC(F)(F)CC3)c(-n3ccc(C4CC4)n3)c2)[nH]1]

### Example 209

The synthesis of the title compound was effected analogously to the synthesis of the compound of Example 208 ((4,4-difluoropiperidin-1-yl )-[4-(5-methyl-4H-1,2,4-triazol-3-yl)-2-[3-(trifluoromethyl)pyrazol-1-yl]phenyl]methanone) wherein 3-(3-cyclopropylpyrazol-1-yl)-4-(4,4-difluoropiperidine-1-carbonypbenzonitrile was used instead of 4-(4,4-difluoropiperidine-1-carbonyl)-3-[3-(trifluoromethyl)pyrazol-1-yl]benzonitrile. The title compound was obtained in a yield of 28% (30 mg).

1H NMR (400 MHz, DMSO) δ 13.88 (s, 1H), 8.11 (d, J=1.5 Hz, 1H), 8.08 (d, J=2.5 Hz, 1H), 7.97 (dd, J=7.9, 1.6 Hz, 1H), 7.49 (d, J=7.9 Hz, 1H), 6.26 (d, J=2.5 Hz, 1H), 3.93-3.78 (m, 1H), 3.52 (t, J=9.2 Hz, 1H), 3.27-3.14 (m, 2H), 2.42 (s, 3H), 1.90 (tt, J=8.4, 5.0 Hz, 4H), 1.76-1.52 (m, 1H), 0.92 (ddd, J=8.3, 3.2, 0.9 Hz, 2H), 0.75-0.68 (m, 1H), 0.67-0.57 (m, 1H); LC-MS: m/z 413.3 (MH+).

Preparation: (4,4-difluoropiperidin-1-yl)-[4-(5-methyl-4H-1,2,4-triazol-3-yl)-2-(3-propan-2-ylpyrazol-1-yl)phenyl]methanone

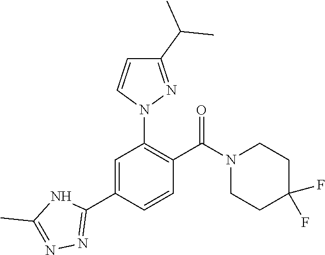 [SMILES: Cc1nnc(-c2ccc(C(=O)N3CCC(F)(F)CC3)c(-n3ccc(C(C)C)n3)c2)[nH]1]

### Example 210

The synthesis of the title compound was effected analogously to the synthesis of the compound of Example 208 ((4,4-difluoropiperidin-1-yl)-[4-(5-methyl-4H-1,2,4-triazol-3-yl)-2[3-(trifluoromethyl)pyrazol-1-yl ]phenyl]methanone) wherein 4-(4,4-difluoropiperidine-1-carbonyl)-3-(3-propan-2-ylpyrazol-1-yl)benzonitrile was used instead of 4-(4,4-difluoropiperidine-1-carbonyl)-3-[3-(trifluoromethyl)pyrazol-1-yl]benzonitrile. The title compound was obtained in a yield of 57% (40 mg).

1H NMR (400 MHz, DMSO) δ 13.88 (s, 1H), 8.12 (d, J=1.5 Hz, 1H), 8.07 (d, J=2.4 Hz, 1H), 7.99 (dd, J=7.9, 1.5 Hz, 1H), 7.50 (d, J=7.9 Hz, 1H), 6.41 (d, J=2.5 Hz, 1H), 3.86- 3.78 (m, 1H), 3.53 (ddd, J=13.3, 8.5, 4.1 Hz, 1H), 3.24 (dt, J=8.2, 4.7 Hz, 1H), 3.20-3.07 (m, 1H), 2.93 (hept, J=6.9 Hz, 1H), 2.42 (s, 3H), 2.06 (s, 1H), 1.87 (s, 2H), 1.64- 1.43 (m, 1H), 1.22 (d, J=6.9 Hz, 6H); 415.3 (MH+).

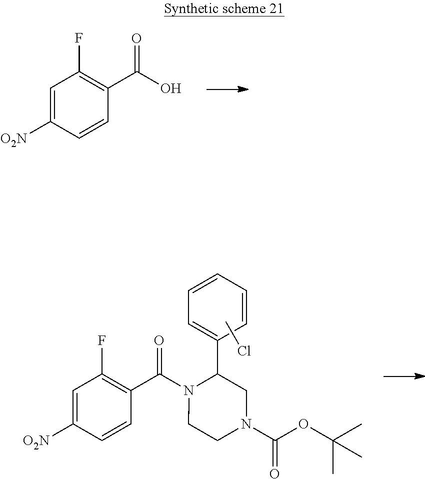

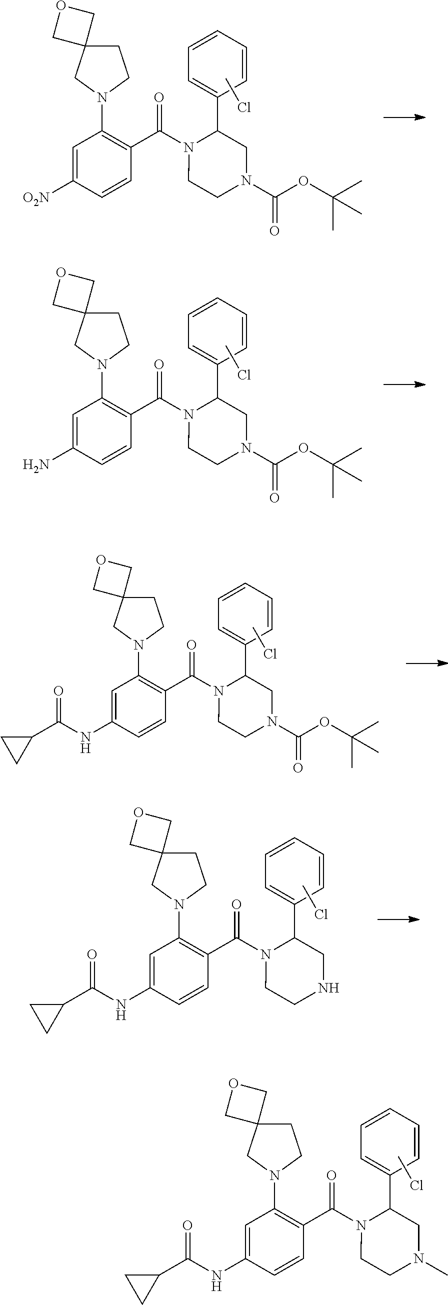

Preparation: tert-butyl 3-(2-chlorophenyl)-4-(2-fluoro-4-nitrobenzoyppiperazine-1-carboxylate

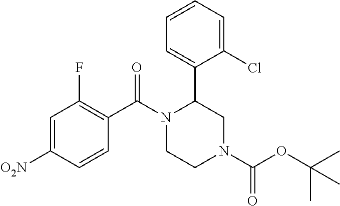 [SMILES: CC(C)(C)OC(=O)N1CCN(C(=O)c2ccc([N+](=O)[O-])cc2F)C(c2ccccc2Cl)C1]

A mixture of 2-fluoro-4-nitrobenzoic acid (300.0 mg, 1.62 mmol) and thionyl dichloride (1.18 mL, 16.21 mmol) was heated to reflux for 1.30 h. After this time the reaction was cooled down to RT and concentrated under vacuum. The residue was dissolved in DCM (0.500 mL) and cooled to 0° C. After 10 min a solution of N,N-Diisopropylethylamine (1.13 mL, 6.48 mmol) and tert-butyl 3-(2-chlorophenyl)piperazine-1-carboxylate (481.0 mg, 1.62 mmol) in DCM (3 mL) was added. The reaction was stirred at RT for overnight. After this time the reaction was diluted with DCM and washed with a s.s. of NaHCO₃ 3 times. The organic phase was washed with Brine, dried over a phase separator and concentrated under vacuum affording the product of formula tert-butyl 3-(2-chlorophenyI)-4-(2-fluoro-4-nitrobenzoyppiperazine-1-carboxylate. The product was used in the next step without further purification.
Yield: 0.74 g

### LC-MS: m/z 408.17 (MH+).

Preparation: tert-butyl 3-(3-chlorophenyl)-4-(2-fluoro-4-nitrobenzoyppiperazine-1-carboxylate

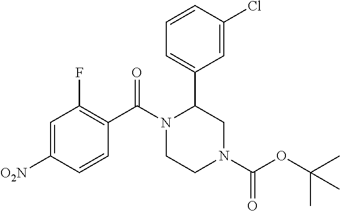 [SMILES: CC(C)(C)OC(=O)N1CCN(C(=O)c2ccc([N+](=O)[O-])cc2F)C(c2cccc(Cl)c2)C1]

The synthesis of the title compound was effected analogously to the synthesis of tert-butyl 3-(2-chlorophenyl)-4-(2-fluoro-4-nitrobenzoyppiperazine-1-carboxylate wherein tert-butyl 3-(3-chlorophenyl)piperazine-1-carboxylate was used instead of tert-butyl 3-(2-chlorophenyl)piperazine-1-carboxylate. The title compound was obtained in a yield of 80% (0.6 g).
LC-MS: m/z 408.17 (MH+).
   Preparation: tert-butyl 3-(2-chlorophenyl)-4-[4-nitro-2-(2-oxa-7-azaspiro[3.4]octan-7-yl)benzoyl]piperazine-1-carboxylate

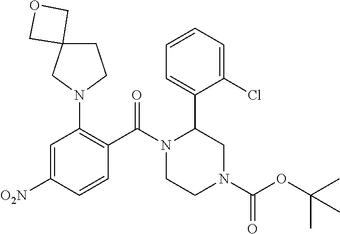 [SMILES: CC(C)(C)OC(=O)N1CCN(C(=O)c2ccc([N+](=O)[O-])cc2N2CCC3(COC3)C2)C(c2ccccc2Cl)C1]

A mixture of tert-butyl 3-(2-chlorophenyl)-4-(2-fluoro-4-nitrobenzoyppiperazine-1-carboxylate (739.0 mg, 1.59 mmol), 2-oxa-6-azaspiro[3.4]octane (270.4 mg, 2.39 mmol) and triethylamine (0.44 mL, 3.19 mmol) in DMSO (13.2 mL) was stirred at 80° C. for 5 h. After this time the reaction was cooled down to RT and diluted with H₂O. The mixture reaction was extracted with AcOEt 3 times. The combined organic fractions were washed with Brine, dried over Na₂SO₄, filtered and concentrated under vacuum affording the product of formula tert-butyl 3-(2-chlorophenyl)-4-[4-nitro-2-(2-oxa-7-azaspiro[3.4]octan yl)benzoyl]piperazine-1-carboxylate. The product was used in the next step without further purification.
Yield: 0.9 g
LC-MS: m/z 557.22 (MH+).
   Preparation: tert-butyl 3-(3-chlorophenyl)-4-[4-nitro-2-(2-oxa-7-azaspiro[3.4]octan-7-yl)benzoyl]piperazine-1-carboxylate

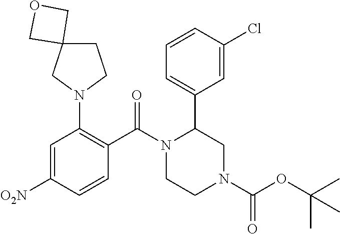 [SMILES: CC(C)(C)OC(=O)N1CCN(C(=O)c2ccc([N+](=O)[O-])cc2N2CCC3(COC3)C2)C(c2cccc(Cl)c2)C1]

The synthesis of the title compound was effected analogously to the synthesis of tert-butyl 3-(2-chlorophenyl)-4-[4-nitro-2-(2-oxa-7-azaspiro[3.4]octan-7-yl)benzoyl]piperazine-1-carboxylate wherein 3-(3-chlorophenyI)-4-(2-fluoro-4-nitrobenzoyl)piperazine-1-carboxylate was used instead of 3-(2-chlorophenyl)-4-(2-fluoro-4-nitrobenzoyl)piperazine-1-carboxylate. The title compound was obtained in a yield of 90% (0.6 g).
LC-MS: m/z 557.22 (MH+).
   Preparation: tert-butyl 4-[4-amino-2-(2-oxa-7-azaspiro[3.4]octan-7-yl)benzoyl]-3-(2-chlorophenyl)piperazine-1-carboxylate

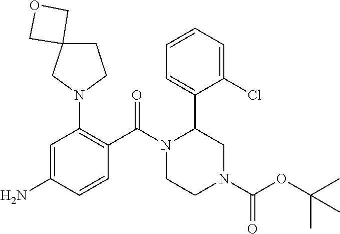 [SMILES: CC(C)(C)OC(=O)N1CCN(C(=O)c2ccc(N)cc2N2CCC3(COC3)C2)C(c2ccccc2Cl)C1]

To a solution of tert-butyl 3-(2-chlorophenyl)-4-[4-nitro-2-(2-oxa-7-azaspiro[3.4]octan-7yl)benzoyl]piperazine-1-carboxylate (1.0 g, 1.788 mmol) in acetic acid (40 mL), zinc (1.62 g, 24.74 mmol) was added. The reaction mixture was stirred at room temperature. After 2h the reaction was filtered on hirsch funnel, washed with EtOAc and concentrated under vacuum. The crude was dissolved in 2.5 ml of MeOH and submitted to a SCX cartridge (20g, washed with MeOH and then eluted with NH3 1N in MeOH). The solvent was evaporated under reduced pressure and the product was purified by FC on silica gel (eluting from 100% of cHex to cHex/AcOEt 20:80), affording the product of formula (tert-butyl 3-(3-chlorophenyl)-4-[4-nitro-2-(2-oxa-7-azaspiro[3.4]octan-7-yl)benzoyl]piperazine-1-carboxylate.
Yield: 802 mg
LC-MS: m/z 527.22 (MH+).
   Preparation: tert-butyl 4-[4-amino-2-(2-oxa-7-azaspiro[3.4]octa n-7-yl)benzoyI]-3-(3-chlorophenyl)piperazine-1-carboxylate

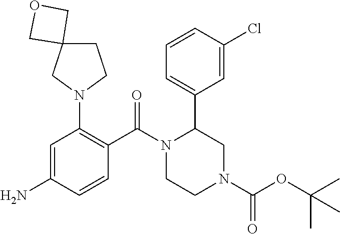 [SMILES: CC(C)(C)OC(=O)N1CCN(C(=O)c2ccc(N)cc2N2CCC3(COC3)C2)C(c2cccc(Cl)c2)C1]

The synthesis of the title compound was effected analogously to the synthesis of the compound of Example tert-butyl 4-[4-amino-2-(2-oxa-7-azaspiro[3.4]octan-7-yl)benzoyl]-3-(2-chlorophenyl)piperazine-1-carboxylate wherein tert-butyl 3-(3-chlorophenyl)-4-[4-nitro-2-(2-oxa-7-azaspiro[3.4]octan-7-yl)benzoyl]piperazine-1-carboxylate was used instead of tert-butyl 3-(2-chlorophenyl)-4-[4-nitro-2-(2-oxa-7-azaspiro[3.4]octan-7-yl)benzoyl]piperazine-1-carboxylate. The title compound was obtained in a yield of 98% (0.6 g).

Preparation: tert-butyl 3-(2-chlorophenyl)-4-[4-(cyclopropanecarbonylamino)-2-(2-oxa-7-azaspiro[3.4]octan-7-yl)benzoyl]piperazine-1-carboxylate

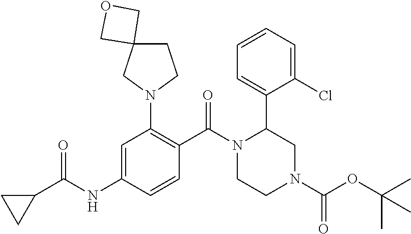 [SMILES: CC(C)(C)OC(=O)N1CCN(C(=O)c2ccc(NC(=O)C3CC3)cc2N2CCC3(COC3)C2)C(c2ccccc2Cl)C1]

To a stirred solution of tert-butyl 4-[4-amino-2-(2-oxa-7-azaspiro[3.4]octan-7-yl)benzoyl]-3-(2-chlorophenyppiperazine-1-carboxylate (0.85 g, 1.522 mmol) and N,N-Diisopropylethylamine (0.53 mL, 3.04 mmol) in DCM (20 mL) at 0° C., cyclopropanecarbonyl chloride (0.2 mL, 1.98 mmol) was added dropwise and the reaction mixture was stirred at room temperature overnight. After this time the mixture was diluted with a s.s. of NaHCO3 and extracted with DCM (X3). The organic phase was washed with brine, dried over a phase separator and concentrated under vacuum. The crude was purified by FC on NH column (eluting from cHex/AcOEt 80:20 to cHex/AcOEt 10:90), affording the product of formula tert-butyl 3-(2-chlorophenyl)-4-[4-(cyclopropanecarbonylamino)-2-(2-oxa-7-azaspiro[3.4]octan-7-yl)benzoyl]piperazine-1-carboxylate.
Yield: 0.5 g
LC-MS: m/z 595.30 (MH+).
   Preparation: tert-butyl 3-(3-chlorophenyl)-4-[4-(cyclopropanecarbonylamino)-2-(2-oxa-7-azaspiro[3.4]octan-7-yl)benzoyl]piperazine-1-carboxylate

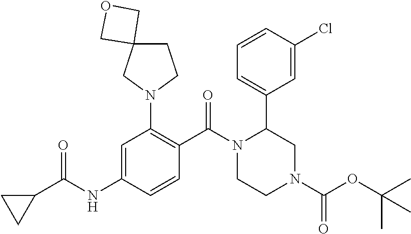 [SMILES: CC(C)(C)OC(=O)N1CCN(C(=O)c2ccc(NC(=O)C3CC3)cc2N2CCC3(COC3)C2)C(c2cccc(Cl)c2)C1]

The synthesis of the title compound was effected analogously to the synthesis of tert-butyl 3-(2-chlorophenyl)-4-[4-(cyclopropanecarbonylamino)-2-(2-oxa-7-azaspiro[3.4]octan-7-yl)benzoyl]piperazine-1-carboxylate wherein tert-butyl 4-[4-amino-2-(2-oxa-7-azaspiro[3.4]octan-7-yl)benzoyl]-3-(3-chlorophenyl)piperazine-1-carboxylate was used instead of tert-butyl 4-[4-amino-2-(2-oxa-7-azaspiro[3.4]octan-7-yl)benzoyI]-3-(2-chlorophenyl)piperazine-1-carboxylate. The title compound was obtained in a yield of 42% (0.3 g).

Preparation: N-[4-[2-(2-chlorophenyl)piperazine-1-carbonyl]-3-(2-oxa-7-azaspiro[3.4]octan-7yl)phenyl]cyclopropanecarboxamide

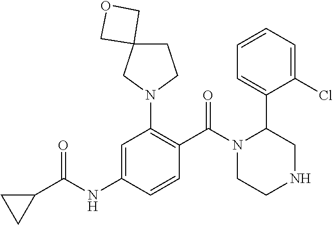 [SMILES: O=C(Nc1ccc(C(=O)N2CCNCC2c2ccccc2Cl)c(N2CCC3(COC3)C2)c1)C1CC1]

To a solution of tert-butyl 3-(2-chlorophenyl)-4-[4-(cyclopropanecarbonylamino)-2-(2-oxa-7-azaspiro[3.4]octan-7-yl)benzoyl]piperazine-1-carboxylate (0.54 g, 0.914 mmol) in DCM (8 mL) Trifluoroacetic acid was added (2 mL, 27.4 mmol). The reaction was stirred at RT for 1 h. After this time the reaction was concentrated under vacuum. The residue was purified by SCX first washing with MeOH then elutin with a solution 1 M of NH3. Basic fractions were collected and concentrated under vacuum. The product was purified by FC on RP using basic conditions conditions (eluting from 100% of ammonium bicarbonate aqueous solution adjusted to pH 10 with ammonia to 100% of CH₃CN) affording the product of formula N-[4-[2-(2-chlorophenyl)piperazine-1-carbonyl]-3-(2-oxa-7-azaspiro[3.4]octan-7-yl)phenyl]cyclopropanecarboxamide.
Yield: 145 mg
LC-MS: m/z 495.31 (MH+).
   Preparation: N-[4-[2-(3-chlorophenyl)piperazine-1-carbonyl]-3-(2-oxa-7-azaspiro[3.4]octan-7-yl)phenyl]cyclopropanecarboxamide

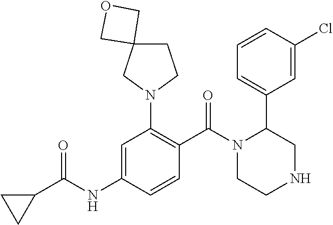 [SMILES: O=C(Nc1ccc(C(=O)N2CCNCC2c2cccc(Cl)c2)c(N2CCC3(COC3)C2)c1)C1CC1]

The synthesis of the title compound was effected analogously to the synthesis of N-[4-[2-(2-chlorophenyppiperazine-1-carbonyl]-3-(2-oxa-7-azaspiro[3.4]octan-7-yl)phenyl]cyclopropanecarboxamide wherein tert-butyl 3-(3-chlorophenyl)-4-[4-(cyclopropanecarbonylamino)-2-(2-oxa-7-azaspiro[3.4]octan-7-yl)benzoyl]piperazine-1-carboxylate was used instead of tert-butyl 3-(2-chlorophenyl)-4-[4-(cyclopropanecarbonylamino)-2-(2-oxa-7-azaspiro[3.4]octan-7-yl)benzoyl]piperazine-1-carboxylate. The title compound was obtained in a yield of 20% (50 mg).
LC-MS: m/z 495.31 (MH+).
   Preparation: N-[4-[2-(2-chlorophenyl)-4-methylpiperazine-1-carbonyl]-3-(2-oxa-7-azaspiro[3.4]octan-7-yl)phenyl]cyclopropanecarboxamide

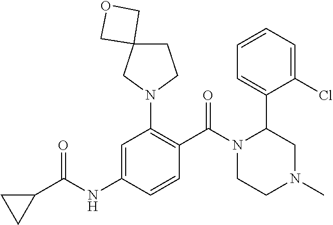 [SMILES: CN1CCN(C(=O)c2ccc(NC(=O)C3CC3)cc2N2CCC3(COC3)C2)C(c2ccccc2Cl)C1]

### Example 211

To a solution of N-[4-[2-(2-chlorophenyppiperazine-1-carbonyl]-3-(2-oxa-7-azaspiro[3.4]octan-7-yl)phenyl]cyclopropanecarboxamide (163 mg, 0.290 mmol) in Methanol (5 mL) was added formaldehyde (235.78 mg, 2.9 mmol) and sodium triacetoxyborohydride (369 mg, 1.74 mmol). The reaction was stirred at room temperature ON. The day after the reaction was concentrated to dryness under reduced pressure and purified by SCX first washing with MeOH and then eluting with 1N NH3 in MeOH. Basic fractions were evaporated and the residue was purified by FC on NH column (eluting from 100% of cHex to cHex/AcOEt 1:1) affording the product of formula N-[4-[2-(2-chlorophenyl)-4-methylpiperazine-1-carbonyl]-3-(2-oxa-7-azaspiro[3.4]octan-7-yl)phenyl]cyclopropanecarboxamide as a racemic mixture.
Yield: 99 mg

1H NMR (400 MHz, DMSO-d6) δ 10.13 (d, 1H), 8.13-7.65 (m, 1H), 7.56-7.23 (m, 3H), 7.21-6.81 (m, 3H), 4.71-4.16 (m, 4H), 3.47 (dt, 3H), 3.25-2.65 (m, 5H), 2.45-2.10 (m, 6H), 2.03-1.71 (m, 3H), 0.79 (s, 4H); LC-MS: m/z 509.23 (MH+).

Preparation: N-[4-[2-(3-chlorophenyl)-4-methylpiperazine-1-carbonyl]-3-(2-oxa-7-azaspiro[3.4]octan-7-yl)phenyl]cyclopropanecarboxamide

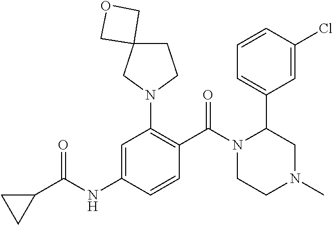 [SMILES: CN1CCN(C(=O)c2ccc(NC(=O)C3CC3)cc2N2CCC3(COC3)C2)C(c2cccc(Cl)c2)C1]

### Example 212

The synthesis of the title compound was effected analogously to the synthesis of the compound of Example N-[4-[2-(2-chlorophenyl)-4-methylpiperazine-1-carbonyl]-3-(2-oxa-7-azaspiro[3.4]octan-7-yl)phenyl]cyclopropanecarboxamide wherein N-[4-[2-(3-chlorophenyl)piperazine-1-carbonyl]-3-(2-oxa-7-azaspiro[3.4]octan-7-yl)phenyl]cyclopropanecarboxamide was used instead of N-[4-[2-(2-chlorophenyl)piperazine-1-carbonyl]-3-(2-oxa-7-azaspiro[3.4]octan-7-yl)phenyl]cyclopropanecarboxamide. The title compound was obtained in a yield of 64% (33 mg).

1H NMR (400 MHz, DMSO-d6) δ 10.15 (d, 1H), 7.92-7.50 (m, 1H), 7.48-7.13 (m, 3H), 7.11-6.82 (m, 2H), 5.86-4.14 (m, 4H), 3.31 (s, 5H), 3.19-2.63 (m, 4H), 2.40-2.12 (m, 6H), 2.06-1.64 (m, 3H), 0.85-0.72 (m, 4H); LC-MS: m/z 509.23 (MH+).

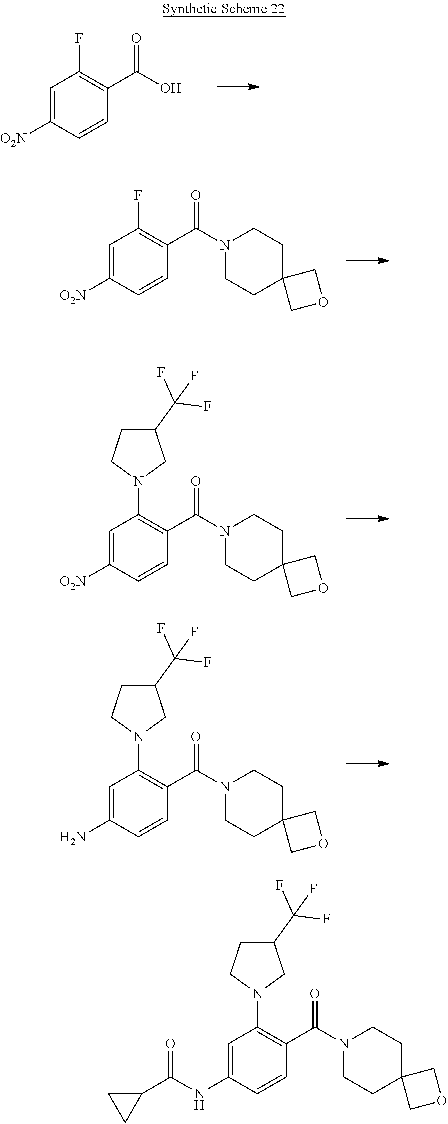

Preparation: (2-fluoro-4-nitrophenyI)-(2-oxa-7-azaspiro[3.5]nonan-7-yl)methanone

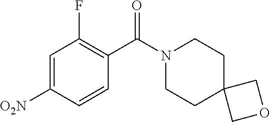 [SMILES: O=C(c1ccc([N+](=O)[O-])cc1F)N1CCC2(CC1)COC2]

A solution of 2-fluoro-4-nitrobenzoic acid (391.0 mg, 2.11 mmol), N,N-Diisopropylethylamine (2.16 mL, 12.67 mmol) and HATU ([dimethylamino(3-triazolo[4,5-b]pyridinyloxy)methylidene]-dimethylammonium hexafluorophosphate (1.2 g, 3.17 mmol)) in DMF (9.11 mL) was stirred for 15 minutes at RT. Then 2-oxa-7-azaspiro[3.5]nonane oxalic acid (436.48 mg, 1.27 mmol) was added and the reaction mixture was stirred at RT overnight. The day after a saturated solution of NaHCO3 was added to the reaction mixture and the aqueous phase was extracted with AcOEt (x3). The organic portions were collected, washed with brine, residual water was removed by Na2SO4, filtered and the solvent was evaporated under reduced pressure. The residue was purified by FC on silica gel (eluting from cHex:AcOEt from 80:20 to 20:80) to give the product of formula (2-fluoro-4-nitrophenyI)-(2-oxa-7-azaspiro[3.5]nonan-7-yl)methanone.
Yield: 545 mg.

1H NMR (400 MHz, DMSO-d6) δ 8.23 (dd, J=9.2, 2.2 Hz, 1H), 8.15 (dd, J=8.4, 2.2 Hz, 1H), 7.71 (dd, J =8.4, 6.7 Hz, 1H), 4.43-4.21 (m, 4H), 3.59 (t, J=5.8 Hz, 2H), 3.13 (t, J=5.6 Hz, 2H), 1.85 (t, J=5.8 Hz, 2H), 1.74 (dd, J=7.0, 4.2 Hz, 2H); LC-MS: m/z 295.1 (MH+).

Preparation: [4-nitro-2-[3-(trifluoromethyl)pyrrolidin-1-yl]phenyl]-(2-oxa-7-azaspiro[3.5]nonan-7-yl)methanone

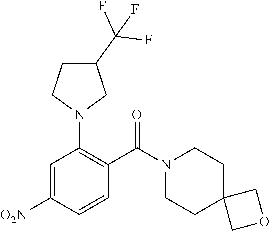 [SMILES: O=C(c1ccc([N+](=O)[O-])cc1N1CCC(C(F)(F)F)C1)N1CCC2(CC1)COC2]

In a proper vial, (2-fluoro-4-nitrophenyl)-(2-oxa-7-azaspiro[3.5]nonan-7-yl)methanone (273.0 mg, 0.930 mmol), N,N-Diisopropylethylamine (0.63 mL, 3.71 mmol), 3-(trifluoromethyl)pyrrolidine hydrochloride (325.77 mg, 1.86 mmol) were dissolved in DMSO (3.981 mL), and stirred at 120° C. for 48 h. After this time the reaction was cooled down to RT and H₂O was added. The mixture was then extracted with AcOEt 3 times. The combined organic fractions were washed with Brine, dried over Na2SO4, filtered and concentrated under vacuum. The residue was purified by FC on RP using acid conditions (eluting from H2O +0.1% of HCOOH/CH₃CN from 90:10 to 50:50) to give the product of formula [4-nitro-2-[3-(trifluoromethyl)pyrrolidin-1-yl]phenyl]-(2-oxa-7-azaspiro[3.5]nonan-7-yl)methanone.
Yield: 138 mg.

1H NMR (400 MHz, DMSO-d6) 57.54 (ddd, J=10.2, 8.3, 2.2 Hz, 1H), 7.45 (dd, J=4.2, 2.2 Hz, 1H), 7.29 (dd, J=8.3, 0.8 Hz, 1H), 4.39-4.27 (m, 4H), 3.74-3.28 (m, 7H), 3.30-3.02 (m, 2H), 2.31-2.18 (m, 1H), 2.11-2.00 (m, 1H), 1.88-1.66 (m, 4H); LC-MS: m/z 414.2 (MH+)

Preparation: [4-amino-2-[3-(trifluoromethyl)pyrrolidin-1-yl]phenyl]-(2-oxa-7-azaspiro[3.5]nonan-7-yl)methanone

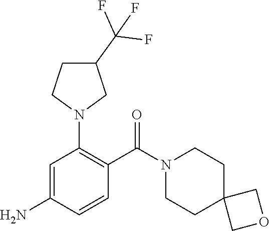 [SMILES: Nc1ccc(C(=O)N2CCC3(CC2)COC3)c(N2CCC(C(F)(F)F)C2)c1]

A mixture of [4-nitro-2-[3-(trifluoromethyl)pyrrolidin-1-yl ]phenyl]-(2-oxa-7-azaspiro[3.5]nonan-7-yl)methanone (138.0 mg, 0.330 mmol) and Pd-C 10% (35.53 mg, 0.030 mmol) was stirred under H₂ atmosphere (1 atm) at RT overnight. After this time the reaction was filtered, concentrated under vacuum affording the product of formula [4-amino-2-[3-(trifluoromethyl)pyrrolidin-1-yl]phenyl]-(2-oxa-7-azaspiro[3.5]nonan-7-yl)methanone. The product was used in the next step without further purification.
Yield: 130 mg.

1H NMR (400 MHz, DMSO-d6) δ 6.68 (d, J=8.1 Hz, 1H), 6.03-5.95 (m, 2H), 5.13 (d, J=7.8 Hz, 2H), 4.34-4.28 (m, 4H), 3.45 (d, J=22.0 Hz, 2H), 3.25 (d, J=21.7 Hz, 7H), 2.14 (dt, J=12.6, 6.3 Hz, 1H), 2.00-1.87 (m, 1H), 1.71 (dd, J=18.3, 9.8 Hz, 4H); LC-MS: m/z 384.2 (MH+).

Preparation: N-[4-(2-oxa-7-azaspiro[3.5]nonane-7-carbonyl)-3-[3-(trifluoromethyl)pyrrolidin-1-yl]phenyl]cyclopropanecarboxamide

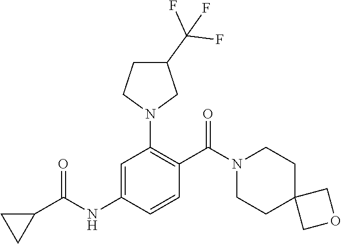 [SMILES: O=C(Nc1ccc(C(=O)N2CCC3(CC2)COC3)c(N2CCC(C(F)(F)F)C2)c1)C1CC1]

### Example 213a, 213b, 213c

To a mixture of [4-amino-2-[3-(trifluoromethyl)pyrrolidin-1-yl]phenyl]-(2-oxa-7-azaspiro[3.5]nonan-7-yl)methanone (130.0 mg, 0.340 mmol)and N,N-Diisopropylethylamine (0.14 mL, 0.850 mmol) in DCM (3.72 mL)at 0° C., cyclopropanecarbonyl chloride (0.04 mL, 0.410 mmol) was added dropwise. The reaction was stopped after 1h at RT and concentrated under vacuum. The residue was purified by FC on RP using basic conditions (eluting with H₂O+0.1% NH₃/CH₃CN from 90:10 to 65:35) evaporation of opportune fractions provided compound of formula N-[4-(2-oxa-7-azaspiro[3.5]nonane-7-carbonyl)-3-[3-(trifluoromethyl)pyrrolidin-1-yl]phenyl]cyclopropanecarboxamide as a racemic mixture.
Yield: 100 mg

1H NMR (400 MHz, DMSO-d6) δ 10.16 (d, J=4.9 Hz, 1H), 7.13 (dd, J=10.1, 1.8 Hz, 1H), 7.00 (td, J=9.5, 8.8, 1.7 Hz, 1H), 6.92 (dd, J=8.2, 4.4 Hz, 1H), 4.37-4.25 (m, 4H), 3.63-3.53(m, 1H), 3.51-3.41 (m, 1H), 3.33 (s, 6H), 3.09 (t, J=5.7 Hz, 1H), 2.18 (dt, J=12.3, 6.3 Hz, 1H), 2.04-1.90 (m, 1H), 1.84-1.60 (m, 5H), 0.81-0.73 (m, 4H); LC-MS: m/z 452.2 (MH+).

The racemic mixture (Example 213c) was then separated into single enantiomers by chiral semi-preparative HPLC

| | Column | Chiralpak IC (25 × 2.0 cm), 5μ | |
|---|---|---|---|
| | Mobile phase | n-Hexane/Ethanol 80/20% v/v | |
| | Flow rate (ml/min) | 17 ml/min | |
| | DAD detection | 220 nm | |
| Preparative chiral | Loop | 500 μL | |
| chromatography | Total amount | 100 mg | |
| protocol: | Injection | 6.25 mg (each injection) | |
| 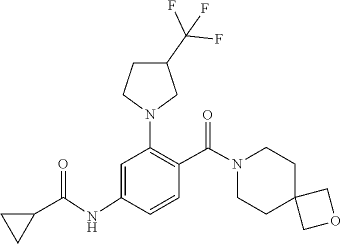 [SMILES: O=C(Nc1ccc(C(=O)N2CCC3(CC2)COC3)c(N2CCC(C(F)(F)F)C2)c1)C1CC1] | Enantiomer 1, Yield: 29.3 mg | Rt 18.80 min | 100% ee |
| (3R or 3S) | LC-MS: m/z 452.48 (MH+) | | |
| | N-[4-(2-oxa-7-azaspiro[3.5]nonane-7-carbonyl)-3-[3R or 3S-(tri- | | |
| | fluoromethyl)pyrrolidin-1yl]phenyl]cyclopropanecarboxamide | | |
| | Example 213a | | |
| 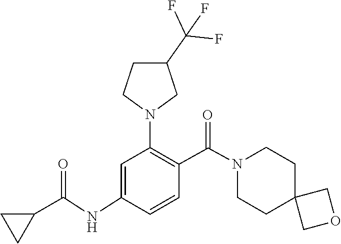 [SMILES: O=C(Nc1ccc(C(=O)N2CCC3(CC2)COC3)c(N2CCC(C(F)(F)F)C2)c1)C1CC1] | Enantiomer 2, Yield: 18 mg | Rt 23.2 min | 100% ee |

| | |
|---|---|
| (3R or 3S) | LC-MS: m/z 452.48 (MH+) |
| | N-[4-(4-methyl-(2R or 2S)-phenylpiperazine-1-carbonyl)- |
| | 3-piperidin-1-ylphenyl]cyclopropanecarboxamide |
| | Example 213b |

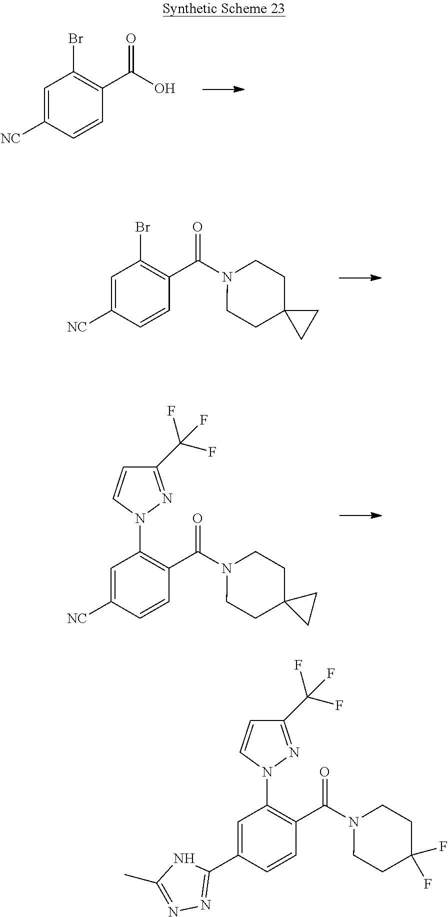

Preparation: 4-(6-azaspiro[2.5]octane-6-carbonyl)-3-bromobenzonitrile

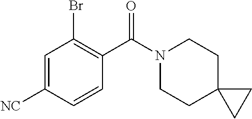 [SMILES: N#Cc1ccc(C(=O)N2CCC3(CC2)CC3)c(Br)c1]

To a solution of [dimethylamino(3-triazolo[4,5-b]pyridinyloxy)methylidene]-dimethylammonium;hexafluorophosphate (841.11 mg, 2.21 mmol),2-bromo-4-cyanobenzoic acid (0.5 g, 2.21 mmol) and 6-azaspiro[2.5]octane hydrochloride (326.62 mg, 2.21 mmol) in DMF (10 mL), N,N-Diisopropylethylamine (857.67 mg, 6.64 mmol) was added. The reaction was stirred at RT for 5 h. After this time the reaction was diluted with H₂O. The mixture was then extracted with AcOEt 3 times. The combined organic fractions were washed with Brine, dried over Na₂SO₄, filtered and concentrated under vacuum. The residue was purified by FC on silica gel (eluting from cHex to AcOEt/cHex 1:1). Pure fraction were collected, concentrated under vacuum affording the product of formula 4-(6-azaspiro[2.5]octane-6-carbonyl)-3-bromobenzonitrile.
Yield: 760 mg

1H NMR (400 MHz, DMSO-d6) δ 8.28 (d, 1H), 7.94 (dd, 1H), 7.57 (d, 1H), 3.67 (t, 2H), 3.14-3.06 (m, 2H), 1.47-1.13 (m, 4H), 0.39-0.24 (m, 4H); LC-MS: 319.14, 321.10 m/z (MH+).

Preparation: 4-(6-azaspiro[2.5]octane-6-carbonyl)-3-[3-(trifluoromethyl)pyrazol-1-yl]benzonitrile

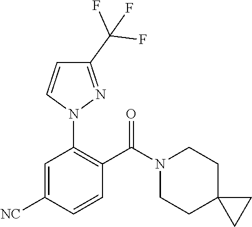 [SMILES: N#Cc1ccc(C(=O)N2CCC3(CC2)CC3)c(-n2ccc(C(F)(F)F)n2)c1]

In a vial 4-(6-azaspiro[2.5]octane-6-carbonyl)-3-bromobenzonitrile (150.0 mg, 0.470 mmol), tripotassium phosphate (212.46 mg, 0.990 mmol),3-(trifluoromethyl)pyrazole (76.74 mg, 0.560 mmol), N,N′-Dimethylcyclohexane-1,2-diamine (6.68 mg, 0.050 mmol) and copper (I) iodide (4.5 mg, 0.020 mmol) were sequentially added . The mixture was suspended in dry 1,4-Dioxane (2.492 mL) and the resulting suspension was degassed bubbling N₂ for 5 minutes and then 3 cycles of vacuum/N₂ were performed. The raction was stirred at 120° C. ON. The day after the reaction mixture was cooled down to RT and diluted with H₂O. The reaction was extracted with AcOEt 3 times. The combined organic fractions were dried over Na₂SO₄, filtered and concentrated under vacuum. The residue was purified by FC on silica gel (eluting from cHex to cHex/AcOEt 4:6) affording the product of formula 4-(6-azaspiro[2.5]octane-6-carbonyl)-3-[3-(trifluoromethyl)pyrazol-1-yl]benzonitrile.
Yield: 146 mg

1H NMR (400 MHz, DMSO-d6) δ 8.51 (dt, 1H), 8.33 (d, 1H), 8.02 (dd, 1H), 7.68 (d, 1H), 7.06 (d, 1H), 3.71-3.60 (m, 1H), 3.45 (td, 1H), 3.23-3.01 (m, 2H), 1.50-0.99 (m, 4H), 0.42-0.13 (m, 4H); LC-MS: m/z 375.1 (MH+).

Preparation: 6-azaspiro[2.5]octan-6-yl-[4-(5-methyl-4H-1,2,4-triazol-3-yl)-2-[3-(trifluoromethyl)pyrazol-1-yl]phenyl]methanone

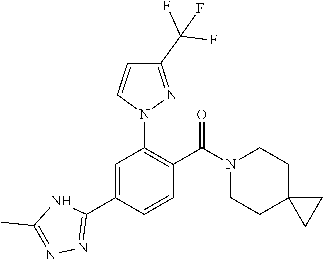 [SMILES: Cc1nnc(-c2ccc(C(=O)N3CCC4(CC3)CC4)c(-n3ccc(C(F)(F)F)n3)c2)[nH]1]

### Example 214

A mixture of 4-(6-azaspiro[2.5]octane-6-carbonyl)-3-[3-(trifluoromethyl)pyrazol-1-yl]benzonitrile (187.0 mg, 0.500 mmol), dicesium carbonate (488.26 mg, 1.5 mmol), ethanimidamide hydrochloride (70.84 mg, 0.750 mmol) and bromocopper (3.58 mg, 0.020 mmol) in DMSO (5 mL) was stirred at 120° C. for 3h. After this time the reaction was cooled down to RT and H₂O was added. The mixture was then extracted with AcOEt 3 times. The combined organic fractions were washed with Brine, dried over Na₂SO₄, filtered and concentrated under vacuum. The residue was purified by FC on NH column (eluting from 100% of DCM to DCM/MOH 9:1) affording the product of formula 6-azaspiro[2.5]octan-6-yl-[4-(5-methyl-4H-1,2,4-triazol-3-yl)-2-[3-(trifluoromethyl)pyrazol-1-yl]phenyl]methanone.
Yield: 38 mg

1H NMR (400 MHz, DMSO-d6) δ 8.46-8.42 (m, 1H), 8.19 (d, 1H), 8.11 (dd, 1H), 7.55 (d, 1H), 7.03 (d, 1H), 3.69 (s, 1H), 3.43 (s, 1H), 3.18 (d, 2H), 2.42 (s, 3H), 1.48-1.00 (m, 4H), 0.41-0.14 (m, 4H); LC-MS: m/z 431.5 (MH+).

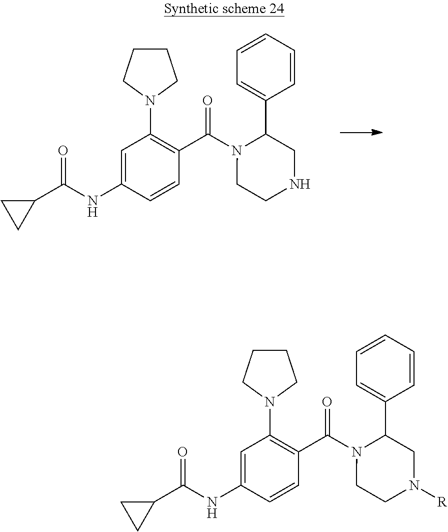

Preparation: N-[4-(4-methylsulfonyl-2-phenylpiperazine-1-carbonyl)-3-pyrrolidin-1-ylphenyl]cyclopropanecarboxamide

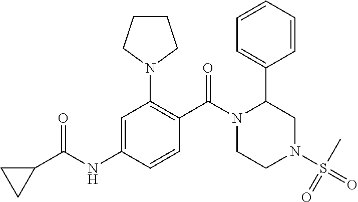 [SMILES: CS(=O)(=O)N1CCN(C(=O)c2ccc(NC(=O)C3CC3)cc2N2CCCC2)C(c2ccccc2)C1]

### Example 215

To a solution of N-[4-(2-phenylpiperazine-1-carbonyl)-3-pyrrolidin-1-yl phenyl]cyclopropanecarboxamide (25.0 mg, 0.060 mmol) in DCM (0.500 mL), triethylamine (0.02 mL, 0.120 mmol) was added followed by methanesulfonyl chloride (0.01 mL, 0.070 mmol). The reaction was stirred at room temperature overnight. The day after the reaction was diluted with water and extracted with DCM (X2). The organic phase was dried using a phase separator and concentrated under reduced pressure. The crude was purified by FC on silica gel (eluting from cHex/AcOEt 50:50 to cHex/AcOEt 20:80) to afford the product of formula N-[4-(4-methylsulfonyl-2-phenylpiperazine-1-carbonyl)-3-pyrrolidin-1-ylphenyl]cyclopropanecarboxamide.
Yield: 21.1 mg

1H NMR (500 MHz, DMSO-d6) δ 10.24-10.04 (m, 1 H), 7.63-7.23 (m, 5 H), 7.22-6.81 (m, 3 H), 6.04-4.79 (m, 1 H), 4.33-3.87 (m, 1 H), 2.97-2.86 (m, 3 H), 4.67-2.73 (m, 5 H), 3.08-2.71 (m, 4 H), 1.83-1.74 (m, 1 H), 2.05-1.60 (m, 4 H), 0.86-0.74 (m, 4 H); LC-MS: m/z 497.25 (MH+).

Preparation: 4-[4-(cyclopropanecarbonylamino)-2-pyrrolidin-1-yl benzoyl]-N,N-dimethyl-3-phenylpiperazine-1-carboxamide

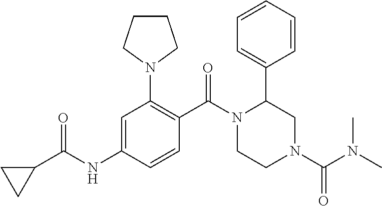 [SMILES: CN(C)C(=O)N1CCN(C(=O)c2ccc(NC(=O)C3CC3)cc2N2CCCC2)C(c2ccccc2)C1]

### Example 216

The synthesis of the title compound was effected analogously to the synthesis of the compound of Example 215 (N-[4-(4-methylsulfonyl-2-phenylpiperazine-1-carbonyl)-3-pyrrolidin-1-ylphenyl]cyclopropanecarboxamide) wherein N,N-dimethylcarbamoyl chloride was used instead of methanesulfonyl chloride. The title compound was obtained in a yield of 77% (0.018 g).

1H NMR (500 MHz, DMSO-d6) δ ppm 10.21-9.96 (m, 1 H), 7.55-6.61 (m, 8 H), 5.90-4.70 (m, 1 H), 4.59-b 2.70 (m, 10 H), 2.73-2.56 (m, 6 H), 2.05-1.47 (m, 5 H), 0.90-0.63 (m, 4 H); LC-MS: m/z 490.29 (MH+).

Preparation: 4-[4-(cyclopropanecarbonylamino)-2-pyrrolidin-1-ylbenzoyl]-N-methyl-3-phenylpiperazine-1-carboxamide

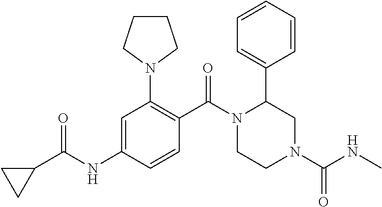 [SMILES: CNC(=O)N1CCN(C(=O)c2ccc(NC(=O)C3CC3)cc2N2CCCC2)C(c2ccccc2)C1]

### Example 217

To a solution of N-[4-(2-phenylpiperazine-1-carbonyl)-3-pyrrolidin-1-ylphenyl]cyclopropanecarboxamide (20.0 mg, 0.050 mmol) in DMF (0.500 6392 L), carbonyldiimidazole (7.75 mg, 0.050 mmol) was added and the reaction was stirred for 2 h at room temperature. Then N,N-Diisopropylethylamine (0.050 mL, 0.288 mmol) and methanamine hydrochloride (19.45 mg, 0.288 mmol) were added and the reaction was at 50° C. ON. The day after the reaction was cooled down to RT, diluted with H₂O and extracted with EtOAc (x3). The organic phase was dried over Na₂SO₄, filtered and concentrated to afford a crude that was purified by semipreparative HPLC: Column CSH C18 (30×100mm, 3 μm) (Conditions: [A1: Waters +0.1% HCOOH]; [131: MeCN]. Gradient: from 33.0% B1 to 34.0% B1 in 10min (flow: 40.00mL/min). Detection: UV/Vis detection range 210 nm to 350nm MS (ES+/ES−) Scan range 100 to 1000 AMU.)
Yield: 6.5 mg

1H NMR (400 MHz, DMSO-d6) δ ppm 10.20-10.02 (m, 1 H), 7.51-6.69 (m, 8 H), 6.55-6.40 (m, 1 H), 5.74-5.56 (m, 1 H), 4.89-4.13 (m, 2 H), 3.77-3.34 (m, 3 H), 3.26-2.81 (m, 5 H), 2.64-2.53 (m, 3 H), 1.98-1.61 (m, 5 H), 0.79 (br s, 4 H); LC-MS: m/z 476.27 (MH+).

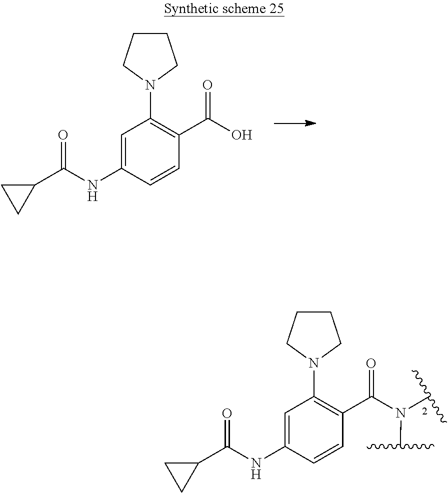 [SMILES: O=C(O)c1ccc(NC(=O)C2CC2)cc1N1CCCC1 ; *N(*)C(=O)c1ccc(NC(=O)C2CC2)cc1N1CCCC1]

Preparation: N-[4-(4-methylsulfonylpiperidine-1-carbonyl)-3-pyrrolidin-1-ylphenyl]cyclopropanecarboxamide

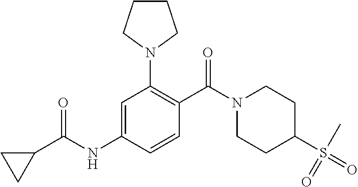 [SMILES: CS(=O)(=O)C1CCN(C(=O)c2ccc(NC(=O)C3CC3)cc2N2CCCC2)CC1]

### Example 218

To a stirred solution of 4-(cyclopropanecarbonylamino)-2-pyrrolidin-1-yl benzoic acid (50.0 mg, 0.180 mmol) in DMF (1.165 mL), N,N-Diisopropylethylamine (0.19 mL, 1.09 mmol) and HATU ([dimethylamino(3-triazolo[4,5-b]pyridinyloxy)methylidene] dimethylammonium hexafluorophosphate (103.96 mg, 0.270 mmol)) were added and the reaction was allowed to stir for 15 minutes at room temperature. Then 4-methylsulfonylpiperidine hydrochloride (43.68 mg, 0.220 mmol) was added and the reaction mixture was stirred at room temperature overnight. A saturated solution of NaHCO₃ was added to the reaction mixture and the aqueous phase was extracted with EtOAc (x3). The organic portions were collected, washed with brine, residual water was removed by Na₂SO₄, filtered and the solvent was evaporated under reduced pressure. Crude was purified by FC on RP using basic conditions (eluting with H₂O+0.1% NH₃/CH₃CN from 95:5 to 70:30) to afford the product of formula N-[4-(4-methylsulfonylpiperidine-1-carbonyl)-3-pyrrolidin-1-ylphenyl]cyclopropanecarboxamide.
Yield: 14 mg

1H NMR (400 MHz, DMSO-d6) δ 10.10 (d, J=8.5 Hz, 1H), 7.13-7.04 (m, 1H), 7.00-6.86 (m, 2H), 4.64 (d, J=12.1 Hz, 1H), 3.63 (s, 1H), 3.44-3.31 (m, 1H), 3.20-3.10 (m, 3H), 2.94 (d, J=1.6 Hz, 5H), 2.75 (q, J=14.2, 13.3 Hz, 1H), 2.12 (d, J=12.9 Hz, 1H), 1.97 (d, J=16.1 Hz, 1H), 1.90-1.82 (m, 4H), 1.76 (tt, J=7.3, 5.1 Hz, 1H), 1.61-1.41 (m, 2H), 0.82-0.73 (m, 4H); LC-MS: m/z 420.5 (MH+).

Preparation: N-[4-(2-oxa-7-azaspiro[3.5]nonane-7-carbonyl)-3-pyrrolidin-1-ylphenyl]cyclopropanecarboxamide

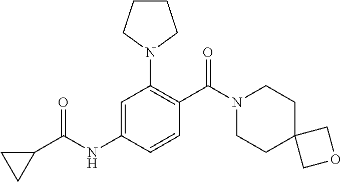 [SMILES: O=C(Nc1ccc(C(=O)N2CCC3(CC2)COC3)c(N2CCCC2)c1)C1CC1]

### Example 219

The synthesis of the title compound was effected analogously to the synthesis of the compound of Example 218 (N-[4-(4-methylsulfonylpiperidine-1-carbonyl)-3-pyrrolidin-1-ylphenyl]cyclopropanecarboxamide), wherein 2-oxa-7-azaspiro[3.5]nonane; oxalic acid was used instead of 4-methylsulfonylpiperidine hydrochloride. The title compound was obtained in a yield of 50% (0.035 g).

1H NMR (400 MHz, DMSO-d6) δ 10.08 (s, 1H), 7.03 (s, 1H), 6.89 (q, J=8.5 Hz, 2H), 4.32 (td, J=15.9, 14.9, 5.8 Hz, 4H), 3.73-3.66 (m, 1H), 3.29-2.95 (m, 7H), 1.94-1.57 (m, 9H), 0.77 (dd, J=6.3, 3.9 Hz, 4H); LC-MS: m/z 384.3 (MH+).

Preparation: N-[4-(6-azaspiro[2.5]octane-6-carbonyl)-3-pyrrolidin-1-ylphenyl]cyclopropanecarboxamide

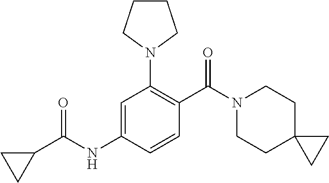 [SMILES: O=C(Nc1ccc(C(=O)N2CCC3(CC2)CC3)c(N2CCCC2)c1)C1CC1]

### Example 220

The synthesis of the title compound was effected analogously to the synthesis of the compound of Example 218 (N-[4-(4-methylsulfonylpiperidine-1-carbonyl)-3-pyrrolidin-1-ylphenyl]cyclopropanecarboxamide) wherein 6-azaspiro[2.5]octane hydrochloride was used instead of 4-methylsulfonylpiperidine hydrochloride. The title compound was obtained in a yield of 51% (0.034 g).

1H NMR (400 MHz, DMSO-d6) δ 10.07 (s, 1H), 7.03 (s, 1H), 6.91 (s, 2H), 3.80-3.72 (m, 1H), 3.48 (t, J=9.4 Hz, 1H), 3.28 (s, 1H), 3.25-3.17 (m, 3H), 3.08 (d, J=6.1 Hz, 2H), 1.91-1.83 (m, 4H), 1.76 (qd, J=7.4, 5.1 Hz, 1H), 1.51-1.10 (m, 4H), 0.78 (q, J=6.8, 5.2 Hz, 4H), 0.38-0.30 (m, 4H); LC-MS: m/z 368.5 (MH+).

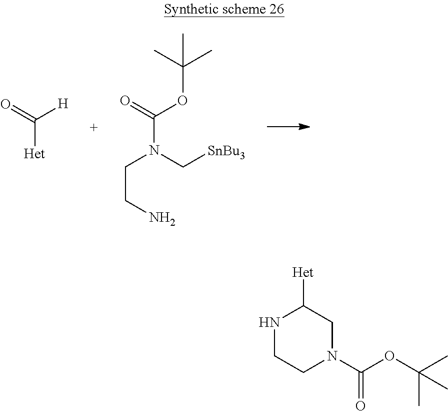

Preparation: tert-butyl 3-pyrimidin-5-ylpiperazine-1-carboxylate

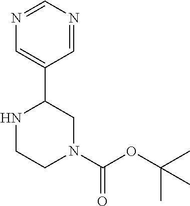 [SMILES: CC(C)(C)OC(=O)N1CCNC(c2cncnc2)C1]

### Example 221

To a solution of SnAP Pip reagent (642.86 mg, 1.39 mmol) in dry DCM (7.05 mL) at room temperature, 5-pyrimidinecarboxaldehyde (150.0 mg, 1.39 mmol) and MS 3A were added. The reaction was stirred at room temperature for 5h and filtered through a short layer of celite (DCM rinse). The filtrate was concentrated under reduced pressure to afford the imine. Separately, 2,6-Lutidine (148.68 mg, 1.39 mmol) was added in one portion to a suspension of dry HFIP (5.5 mL, 1.39 mmol) and anhydrous Copper(II) trifluoromethanesulfonate (501.87 mg, 1.39 mmol) and stirred at room temperature for 1h, during which time a homogeneous suspension was formed. A solution of imine in dry DCM (22.5 mL) was added in one portion and the resulting mixture was stirred at room temperature overnight. After 18 h the reaction was quenched with 10% aq NH₄OH (4 mL) and stirred vigorously for 15 min. The layers were separated and the aqueous layer was extracted with DCM x3. The combined organic layers were washed with H₂O x3 and brine x1, dried using a phase separator and concentrated. The crude was purified by column chromatography (Sfar C18 12g, eluent: from H2O+NH4OH 0.1% 100% to H20+NH4OH 0.1%/AcCN 80:20) affording the product of formula tert-butyl 3-pyrimidin-5-ylpiperazine-1-carboxylate.
Yield: 0.090 g

¹ NMR (400 MHz, DMSO-d₆) δ ppm 10.20 (s, 1 H), 9.92 (s, 2 H), 5.11-4.73 (m, 3 H), 4.23-3.68-4.23 (m, 5 H), 2.50 (s, 9 H); LC-MS: m/z 265.12 (MH+).

Preparation: tert-butyl 3-(3-chloropyridin-4-yl)piperazine-1-carboxylate

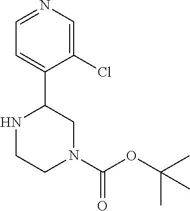 [SMILES: CC(C)(C)OC(=O)N1CCNC(c2ccncc2Cl)C1]

### Example 222

The synthesis of the title compound was effected analogously to the synthesis of the compound of Example 221 (tert-butyl 3-pyrimidin-5-ylpiperazine-1-carboxylate), wherein 3-chloro-4-pyridinecarboxaldehyde was used instead 5-pyrimidinecarboxaldehyde. The title compound was obtained in a yield of 37% (0.116 g).

¹ NMR (400 MHz, DMSO-d₆) δ ppm 8.60 (s, 1 H), 8.57-8.50 (m, 1 H), 7.70-7.61 (m, 1 H), 3.72 (br t, J=5.83 Hz, 4 H), 3.08-2.62 (m, 4 H), 1.53-1.36 (m, 9 H); LC-MS: m/z 298.11 (MH+).

Preparation: tert-butyl 3-pyridin-3-ylpiperazine-1-carboxylate

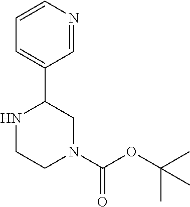 [SMILES: CC(C)(C)OC(=O)N1CCNC(c2cccnc2)C1]

### Example 223

The synthesis of the title compound was effected analogously to the synthesis of the compound of Example 221 (tert-butyl 3-pyrimidin-5-ylpiperazine-1-carboxylate), wherein 3-pyridinecarboxaldehyde was used instead 5-pyrimidinecarboxaldehyde. The title compound was obtained in a yield of 32% (0.118 g).

¹ NMR (400 MHz, DMSO-d₆) δ ppm 8.65-8.58 (m, 1 H), 8.52-8.46 (m, 1 H), 7.84-7.78 (m, 1 H), 7.42-7.31 (m, 1 H), 3.93-3.76 (m, 2 H), 3.69-3.58 (m, 1 H), 3.28 (s, 1 H), 3.00-2.57 (m, 4 H), 1.41 (s, 9 H); LC-MS: m/z 264.36 (MH+).

Preparation: N-[4-[2-(5-chloropyridin-3-y0-4-methylpiperazine-1-carbonyl]-3-pyrrolidin-1-ylphenyl]cyclopropanecarboxamide

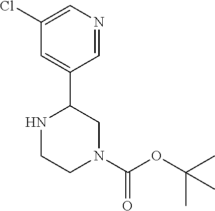 [SMILES: CC(C)(C)OC(=O)N1CCNC(c2cncc(Cl)c2)C1]

### Example 224

The synthesis of the title compound was effected analogously to the synthesis of the compound of Example 221 (tert-butyl 3-pyrimidin-5-ylpiperazine-1-carboxylate), wherein 5-chloronicotinaldehyde was used instead 5-pyrimidinecarboxaldehyde. The title compound was obtained in a yield of 45% (0.067 g).

¹ NMR (400 MHz, DMSO-d₆) δ ppm 8.61-8.51 (m, 2 H), 7.97-7.91 (m, 1 H), 3.93-3.65 (m, 3 H), 3.05-2.58 (m, 5 H), 1.41 (s, 9 H); LC-MS: m/z 298.13 (MH+).

Hypertension

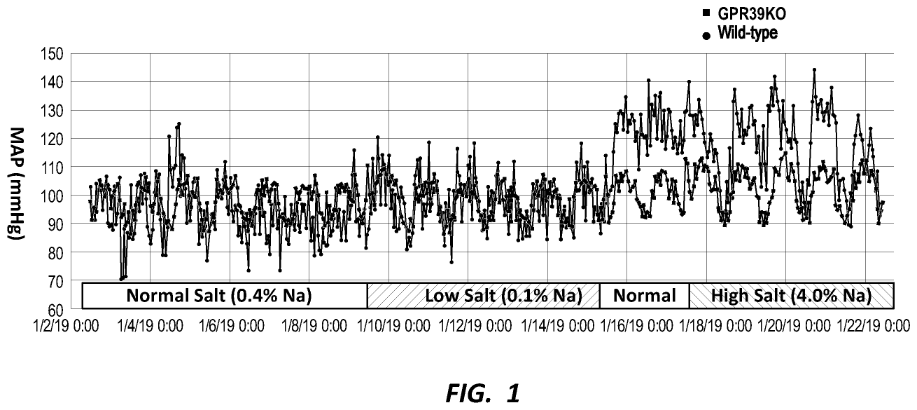

To determine if GPR39 gene deletion alters blood pressure (BP), BP was measured in GPR39 knockout mice (generated by Cyagen) and their wild-type (WT) littermates using radiotelemetry , allowing continuous BP monitoring over weeks in conscious, unrestrained mice. FIG. 1 shows circadian fluctuations in MAPs (hourly), with no difference between the two groups when fed normal (0.4% Na) or sodium deficient (0.1% Na) chow. However, when placed on high-salt diet (4.0% NaCI added), GPR39 KO mice (n=10) were protected from the increased BP induced by high salt diet in WT mice (Combe R, Mudgett J, El Fertak L, Champy MF, Ayme-Dietrich E, Petit-Demoulière B, Sorg T, Herault Y, Madwed JB, Monassier L. How Does Circadian Rhythm Impact Salt Sensitivity of BP in Mice? A Study in Two Close C57131/6 Substrains. PLoS One. 2016 April 18;11(4):e0153472)(the BP in WT mice increased transiently when the diet was switched from low- to high-salt diet). The overall increase in BP in WT mice averaged 12.6±6.0 mmHg (relative to baseline, n=5) compared to 5.0±1.3 mmHg (n=10) in GPR39 KO mice (p=0.075). The resistance of GPR39 KO mice to salt-induced hypertension provides a rationale for the develoμment of GPR39 inhibitors for the treatment of hypertension, especially salt-sensitive hypertension, where the role of Epoxyeicosatrienoic acids (EETs) has been well documented

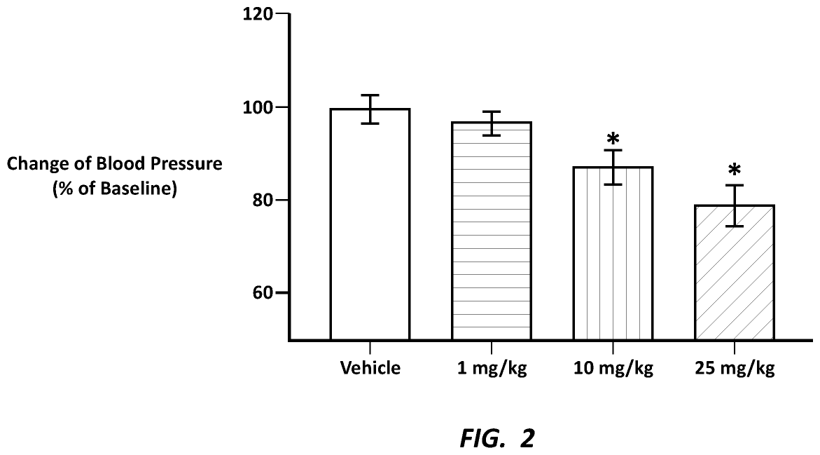

The effect of acute intravenous administration of EXAMPLE 34 on BP in the spontaneously hypertensive rat (SHR) was determined. FIG. 2 shows that at concentrations higher than 10 mg/kg, EXAMPLE 34 significantly reduced BP in SHR (measured through a femoral artery catheter under isoflurane anaesthesia). At 10 mg/kg, BP was reduced by 15%, and at 25 mg/kg, the reduction exceeded 20% of pre-administration baseline. EXAMPLE 34 was administered slowly via bolus injections through the jugular vein in a vehicle consisting of 100% PEG 400, and the change in BP after administration (lowest value over 10 min) was normalized to pre-administration baseline. This was the first proof of principle that pharmacological inhibition of GPR39 reduced BP acutely in a well-established animal model of hypertension.

Heart Attack (acute myocardial infarction)

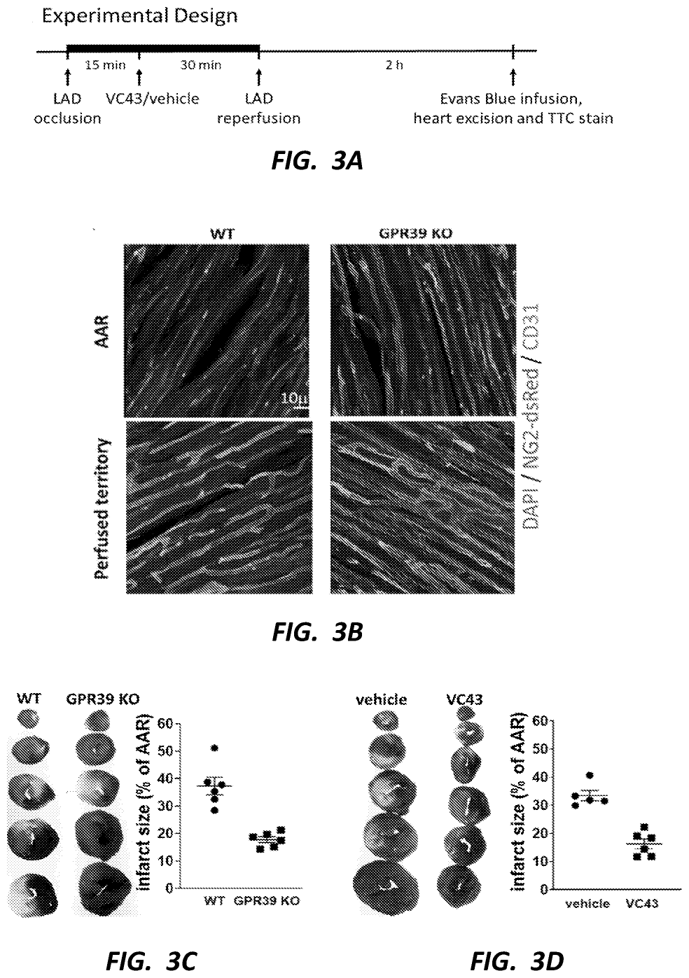

The role of pericytes in tissue necrosis following coronary occlusion was examined (FIG. 3A). Whereas, wild-type NG2-dsRed mice exposed to a 45 min occlusion of the left coronary artery followed by 2 hr reperfusion showed a significant decrease in the number of healthy perfused capillaries, as shown by CD31 immuno-labeling of the endothelium, capillary perfusion was significantly enhanced in global GPR39 knockout mice (FIG. 3B). The infarct size/area at risk (AAR) ratio was also significantly smaller in global GPR39 knockout mice (FIG. 3C) and those treated with EXAMPLE 34 (FIG. 3D).

No reflow

This phenomenon describes lack of microvascular perfusion despite an open artery during a heart attack and is associated with poor outcome.

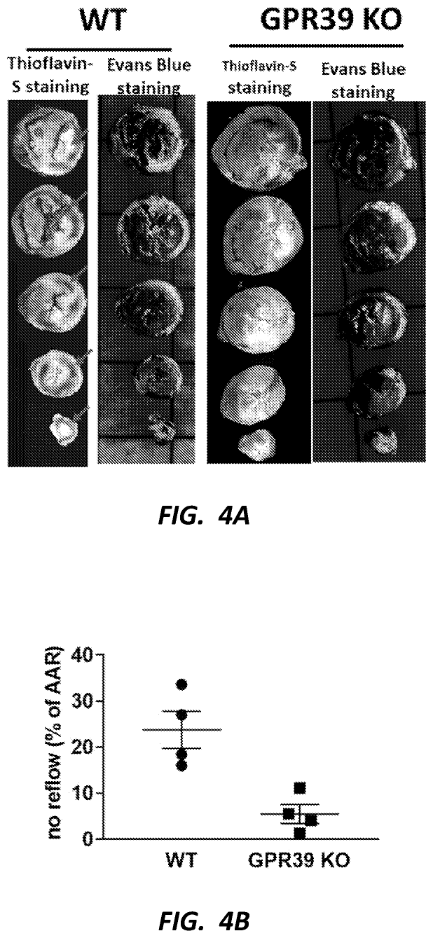

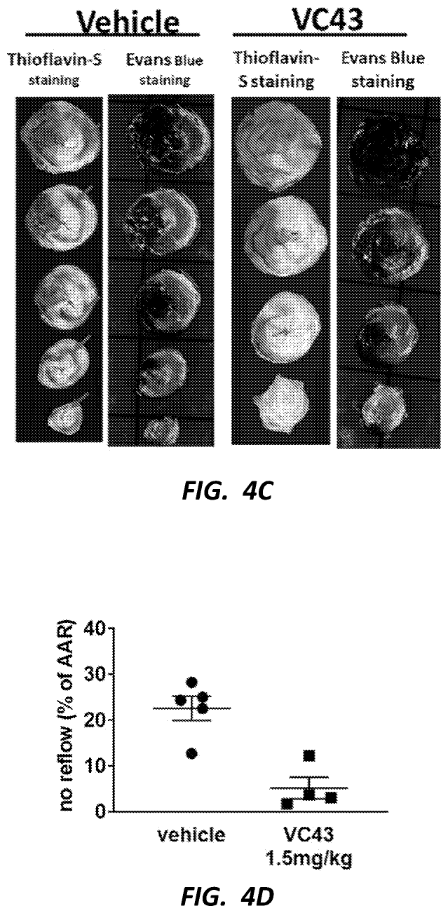

The role of pericytes was examined in no reflow after reperfusion in a separate group of animals since infarct size and no reflow post-mortem cciuid not be measured in the same animals, Thioflavin S, a perfusion marker, was injected into the left coronary artery after reperfusion. Thereafter, the left coronary artery was briefly re-occluded and 0.2 ml Evan's blue (1%) injected. The heart was then excised, transversely sliced into 5-6 pieces and photographed under UV light (under which Thioflavin S fluoresces blue) to identify the area of no reflow and under standard lighting to identify the AAR, and the no reflow zone expressed as a percent of the AAR. The global GPR39 knockout mice and wild-type mice treated with EXAMPLE 34 (1.5 mg/kg i.v.) showed negligible no reflow zones compared to controls (FIGS. 4A-D).

What is claimed:

1. A compound of Formula (I), or a pharmaceutically acceptable salt thereof: 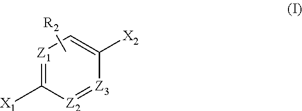
wherein:
X₁ is selected from the group of: 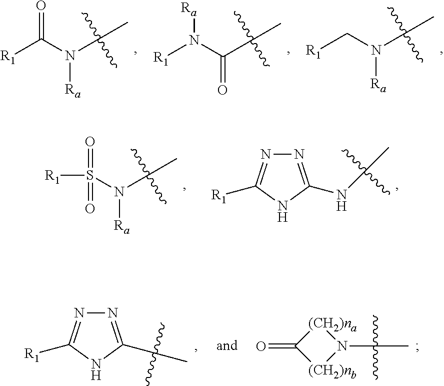
nₐ is an integer selected from the group of 0, 1, and 2;
n_{b} is an integer selected from the group of 0, 1, 2, 3, and 4;
with the proviso that the sum of nₐ+n_{b}is not less than 2 and not greater than 4;
or X₁ and Z₁ together form a fused ring system of the formula (la): 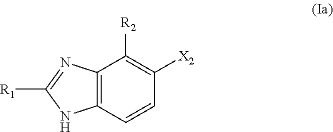
Rₐ is selected from the group of hydrogen and C₁-C₃ alkyl;
X₂ is selected from the group of: 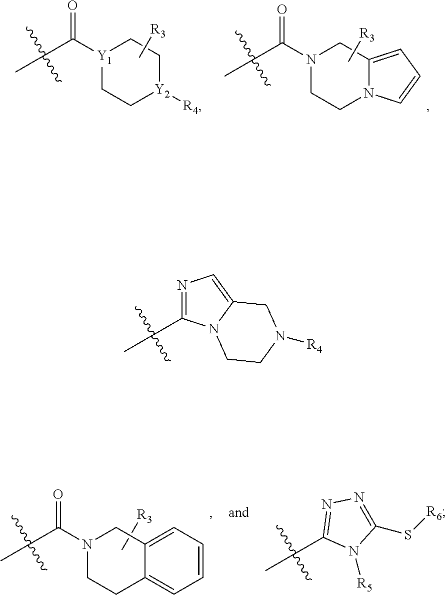
the wavy line [symbol] in each instance represents a bond through which each X₁ and X₂ moiety is bound;
Y₁is selected from the group of C and N;
Y₂ is selected from the group of C, N, S, and O, provided R₄ is not present when Y₂ is O and provided R₄ is either not present or present one or two times when Y₂ is S;
with the proviso that no more than one of Y₁and Y₂ are C;
Z₁; Z₂, and Z₃ are each independently selected from the group of C and N, with the proviso that no more than two of Z₁, Z₂, and Z₃ may be N, and with the further proviso that Z₁; Z₂, and Z₃, when bound to R₂, are C;
R₁ is selected from the group of C₁-C₆ alkyl, —(CH₂)ₙ₁-C₃-C₆cycloalkyl, —NR^{x}R^{y}, phenyl, and benzyl, wherein the C₁-C₆ alkyl group and the rings of the —(CH₂)ₙ₁-C₃-C₆cycloalkyl, phenyl, and benzyl groups are substituted by 0, 1, 2, or 3 substituents selected from the group of halogen, OH, CF₃, and —O—C₁-C₃ alkyl, and wherein each of R^{x} and R^{Y} are independently selected from the group of H and C₁-C₆ alkyl substituted by 0, 1, 2, or 3 substituents selected from the group of halogen, OH, CF₃, and —O—C₁-C₃ alkyl;
n1 is an integer selected from the group of 0, 1, 2, and 3;
R₂ is selected from the group of phenyl and a monocyclic heterocyclic ring or a bicyclic or spirocyclic heterocyclic ring system bound through a nitrogen heteroatom and containing 3, 4, 5, 6, 7, or 8 ring carbon atoms and 0, 1, 2, 3, or 4 additional ring heteroatom selected from the group of N and O, with the R₂ monocyclic ring or a bicyclic or spirocyclic ring system substituted by 0, 1, 2, or 3 substituents selected from the group of C₁-C₆ alkyl, —O—C₁-C₆ alkyl, —(CH₂)ₙ₁-C₃-C₆cycloalkyl, —CF₃, halogen, and phenyl;
R₃ is present one or more times and is independently selected from the group of:
a) hydrogen;
b) —CO₂H or —CO₂—(C₁-C₆ alkyl);
c) C₁-C₆ alkyl substituted by 0, 1, 2, 3, 4 or 5 substituents selected from the group of halogen, CF₃, and OH;
d) phenyl, benzyl, C₃-C₆ cycloalkyl, and —CH₂-C₃-C₆ cycloalkyl, the rings of each of the phenyl, benzyl, C₃-C₆ cycloalkyl, and —CH₂-C₃-C₆ cycloalkyl groups being substituted by 0, 1, 2, or 3 substituents selected from OH, halogen, and C₁-C₆ alkyl, wherein the C₁-C₆ alkyl group is further substituted by 0, 1, 2, 3, 4 or 5 substituents selected from the group of halogen, —CF₃, and OH;
e) a 5-membered or 6-membered heterocyclic ring containing 1, 2, or 3 ring heteroatoms independently selected from O, S, and N, the 5-membered or 6-membered heterocyclic ring being substituted by 0, 1, 2, or 3 substituents selected from OH, halogen, benzyl, and C₁-C₆ alkyl, wherein the C₁-C₆ alkyl group is further substituted by 0, 1, 2, 3, 4 or 5 substituents selected from the group of halogen and OH;
R₄ is present one or two times and is independently selected from the group of H, oxo, C₁-C₆ alkyl,—(CH₂)ₙ₂—C₃-C₆ cycloalkyl, —O—(CH₂)ₙ₂—C₃-C₆ cycloalkyl, —(═O)—O—C₁-C₆ alkyl, —S(═O)₂-C₁-C₆ alkyl, —C(═O)—NR^{X}R^{Y}, phenyl, benzyl, or a heterocyclic ring having 3, 4, 5, 6, 7, 8, 9, or 10 ring atoms of which 1, 2, 3, or 4 ring atoms are selected from the group of N, O, and S, wherein each of R^{x} and R^{Y} are independently selected from the group of H and C₁-C₆ alkyl substituted by 0, 1, 2, or 3 substituents selected from halogen, OH, CF₃, and —O—C₁-C₃ alkyl;
and, when Y₂ is carbon, R₄ may also be —O—C₁-C₆ alkyl or two R₄ may form a carbocycle or heterocycle;
wherein the R₄ C₁-C₆ alkyl, —(═O)—O—C₁-C₆ alkyl, and —O—C₁-C₆ alkyl groups and the rings of the —(CH₂)ₙ₂—C₃-C₆cycloalkyl and —O—(CH₂)ₙ₂-C₃-C₆cycloalkyl, phenyl, and benzyl groups are each independently substituted by 0, 1, 2, or 3 substituents selected from the group of halogen, CF₃, OH, a heterocyclic ring having 3, 4, 5, 6, 7, 8, 9, or 10 ring atoms of which 1, 2, 3, or 4 ring atoms are selected from the group of N, O, and S, substituted or unsubstituted phenyl, and —O—C₁-C₃ alkyl, with the proviso that when Y₁is nitrogen and Y₂ is carbon, then R₄ is not unsubstituted benzyl and with the proviso that when Y₁is nitrogen and Y₂ is nitrogen, then R₄ is not unsubstituted pyridinyl or substituted or unsubstituted phenyl;
R₅ is selected from the group of H and C₁-C₆ alkyl, wherein the R₅ C₁-C₆ alkyl group is further substituted by 0, 1, 2, 3, 4 or 5 substituents selected from the group of halogen, —CF₃, —NR^{x}R^{y}, and OH, wherein each of R^{X} and R^{Y} are independently selected from the group of H and C₁-C₆ alkyl substituted by 0, 1, 2, or 3 substituents selected from the group of halogen, OH, CF₃, and —O—C₁-C₃ alkyl;
R₆ is selected from the group of H, C₁-C₆ alkyl, a heterocyclic ring having 3, 4, 5, 6, 7, 8, 9, or 10 ring atoms of which 1, 2, 3, or 4 ring atoms are selected from the group of N, O, and S, phenyl, and benzyl, wherein the R₆ C₁-C₆ alkyl groups is further substituted by 0, 1, 2, 3, 4 or 5 substituents selected from the group of halogen, —CF₃, -NRₓR^{y}, and OH, and the rings of the R₆ phenyl and benzyl groups and the heterocyclic ring are substituted by 0, 1, 2, 3, 4 or 5 substituents selected from the group of C₁-C₆ alkyl, —O—C₁-C₆ alkyl, halogen, —CF₃, and OH, wherein each of R^{x} and R^{y} are independently selected from the group of H and C₁-C₆ alkyl substituted by 0, 1, 2, or 3 substituents selected from halogen, OH, CF₃, and —O—C₃ alkyl; and
n₂ in each instance is an integer selected from the group of 0, 1, 2, and 3;
with the proviso that, when R₂ is an unsubstituted azepanyl ring ortho to X₂, X₁ is the moiety 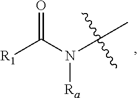
Rₐ is H, R₁ is unsubstituted cyclopropyl, X₂ is 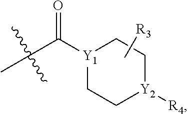
and Y₁is N, and either a) Y₂ is O, b) Y2 is N and R₄ is H or alkyl, c) Y₂ is C and R₄ is H, alkyl, or −C(═O)—O—C₁-C₆ alkyl, then R₃ is not H.

2. The compound of claim 1, wherein compound is of Formula (I-A): 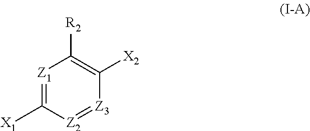
and wherein X₁, X₂, R₂, Z₁, Z₂, and Z₃ are as defined in claim 1; or a pharmaceutically acceptable salt thereof.

3. The compound of claim 1, wherein compound is of Formula (I-13): 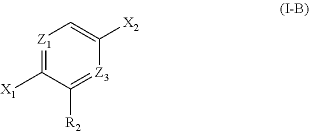
and wherein X₁, X₂, R₂, Z₁, and Z₃ are as defined in claim 1; or a pharmaceutically acceptable salt thereof.

4. A compound of any of claims 1 and 2, or a pharmaceutically acceptable salt thereof, having the formula: 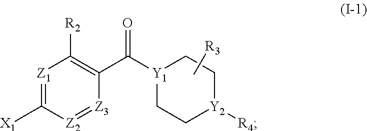
wherein, X₁, Z₁, Z₂, Z₃, Y₁, Y₂, R₂, R₃, and R₄ are as defined in claim 1.

5. A compound of any of claims 1 and 2, or a pharmaceutically acceptable salt thereof, having the formula: 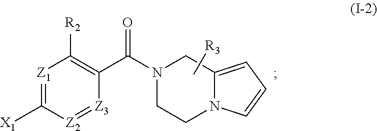
wherein, X₁, Z₁, Z₂, Z₃, R₂, and R₃ are as defined in claim 1.

6. A compound of any of claims 1 and 2, or a pharmaceutically acceptable salt thereof, having the formula: 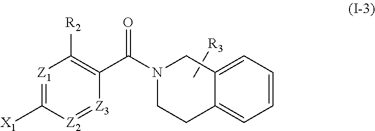
wherein X₁, Z₁, Z₂, Z₃, R₂, and R₃ are as defined in claim 1.

7. A compound of any of claims 1 and 2, or a pharmaceutically acceptable salt thereof, having the formula: 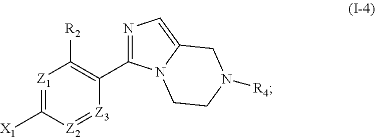
wherein, X₁, Z₁, Z₂, Z₃, R₂, and R₄ are as defined in claim 1.

8. A compound of any of claims 1 and 2, or a pharmaceutically acceptable salt thereof, having the formula: 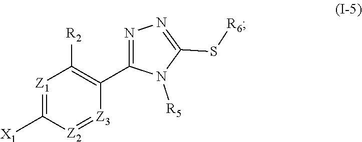
wherein, X₁, Z₁, Z₂, Z₃, R₂, R₄, R₅, and R₆ are as defined in claim 1.

9. A compound of any of claims 1 and 2, or a pharmaceutically acceptable salt thereof, having the formula: 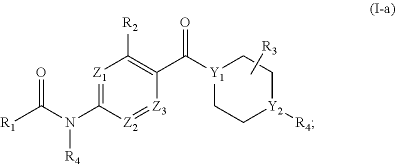
wherein, Z₁, Z₂, Z₃, Y₁, Y₂, Rₐ, R₁, R₂, R₃, and R₄ are as defined in claim 1.

10. A compound of any of claims 1 and 2, or a pharmaceutically acceptable salt thereof, having the formula: 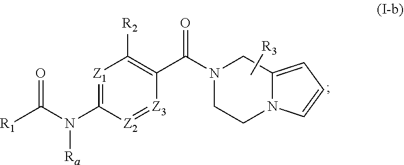
wherein, Z₁, Z₂, Z₃, Rₐ, R₁, R₂, and R₃ are as defined in claim 1.

11. A compound of any of claims 1 and 2, or a pharmaceutically acceptable salt thereof, having the formula: 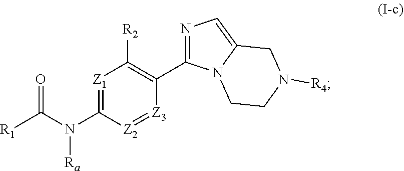
wherein, Z₁, Z₂, Z₃, Rₐ, R₁, R₂, and R₄ are as defined in claim 1.

12. A compound of any of claims 1 and 2, or a pharmaceutically acceptable salt thereof, having the formula: 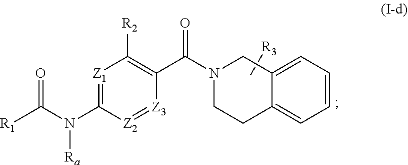
wherein, Z₁, Z₂, Z₃, Rₐ, R₁, R₂, and R₃ are as defined in claim 1.

13. A compound of any of claims 1 and 2, or a pharmaceutically acceptable salt thereof, having the formula: 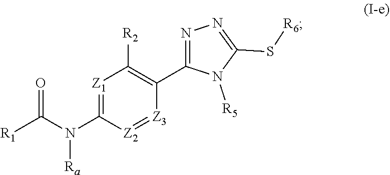
wherein, Z₁, Z₂, Z₃, Rₐ, R₁, R₂, R₅, and R₆ are as defined in claim 1.

14. A compound of any of claims 1 and 2, or a pharmaceutically acceptable salt thereof, having the formula: 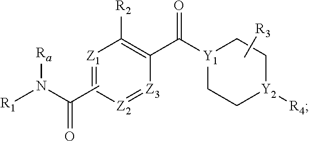
wherein, Z₁, Z₂, Z₃, Y₁, Y₂,Rₐ, R₁, R₂, R₃, and R₄ are as defined in claim 1.

15. A compound of any of claims 1 and 2, or a pharmaceutically acceptable salt thereof, having the formula: 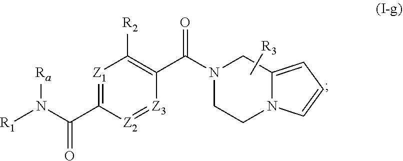
wherein, Z₁, Z₂, Z₃, Rₐ, R₁, R₂, and R₃ are as defined in claim 1.

16. A compound of any of claims 1 and 2, or a pharmaceutically acceptable salt thereof, having the formula: 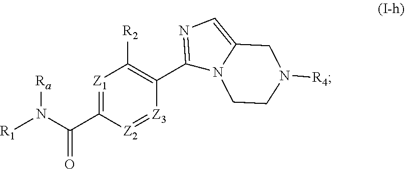
wherein, Z₁, Z₂, Z₃, Rₐ, R₁, R₂, and R₄ are as defined in claim 1.

17. A compound of any of claims 1 and 2, or a pharmaceutically acceptable salt thereof, having the formula: 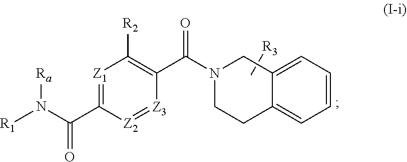
wherein, Z₁, Z₂, Z₃, Rₐ, R₁, R₂, and R₄ are as defined in claim 1.

18. A compound of any of claims 1 and 2, or a pharmaceutically acceptable salt thereof, having the formula: 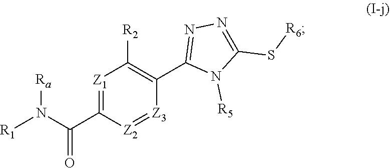
wherein, Z₁, Z₂, Z₃, Rₐ, R₁, R₂, R₅, and R₆ are as defined in claim 1.

19. A compound of any of claims 1 and 2, or a pharmaceutically acceptable salt thereof, having the formula: 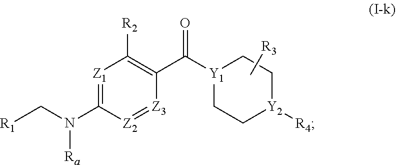
wherein, Z₁, Z₂, Z₃, Y₁, Y₂,Rₐ, R₁, R₂, and R₄ are as defined in claim 1.

20. A compound of any of claims 1 and 2, or a pharmaceutically acceptable salt thereof, having the formula: 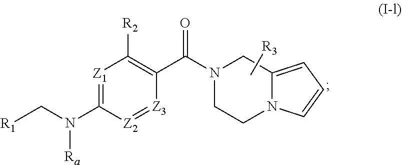
wherein, Z₁, Z₂, Z₃, Rₐ, R₁, R₂, and R₃ are as defined in claim 1.

21. A compound of any of claims 1 and 2, or a pharmaceutically acceptable salt thereof, having the formula: 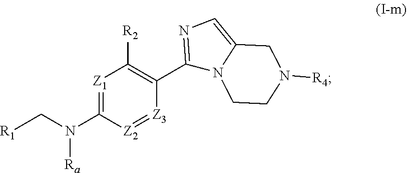
wherein, Z₁, Z₂, Z₃, Rₐ, R₁, R₂, and R₄ are as defined in claim 1.

22. A compound of any of claims 1 and 2, or a pharmaceutically acceptable salt thereof, having the formula: 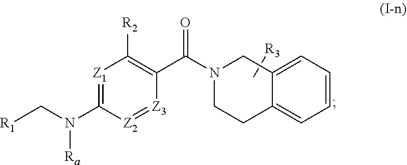
wherein, Z₁, Z₂, Z₃, Rₐ, R₁, R₂, and R₃ are as defined in claim 1.

23. A compound of any of claims 1 and 2, or a pharmaceutically acceptable salt thereof, having the formula: 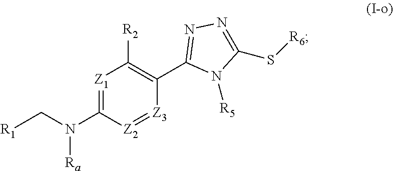
wherein, Z₁, Z₂, Z₃, Rₐ, R₁, R₂, R₅, and R₆ are as defined in claim 1.

24. A compound of any of claims 1 and 2, or a pharmaceutically acceptable salt thereof, having the formula: 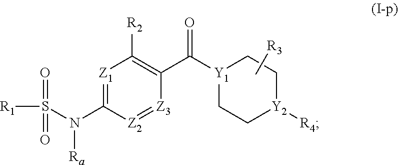
wherein, Z₁, Z₂, Z₃, Y₁, Y₂,Rₐ, R₁, R₂, R₃, and R₄ are as defined in claim 1.

25. A compound of any of claims 1 and 2, or a pharmaceutically acceptable salt thereof, having the formula: 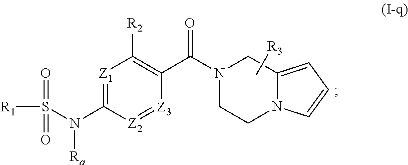
wherein, Z₁, Z₂, Z₃, Rₐ, R₁, R₂, and R₃ are as defined in claim 1.

26. A compound of any of claims 1 and 2, or a pharmaceutically acceptable salt thereof, having the formula: 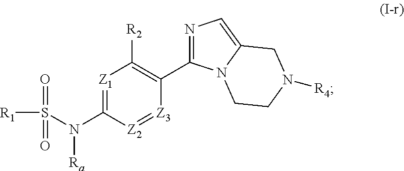
wherein, Z₁, Z₂, Z₃, Rₐ, R₁, R₂, and R₄ are as defined in claim 1.

27. A compound of any of claims 1 and 2, or a pharmaceutically acceptable salt thereof, having the formula: 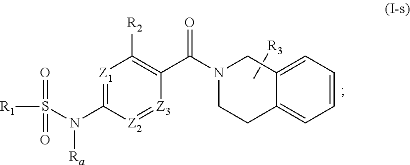
wherein, Z₁, Z₂, Z₃, Rₐ, R₁, R₂, and R₃ are as defined in claim 1.

28. A compound of any of claims 1 and 2, or a pharmaceutically acceptable salt thereof, having the formula: 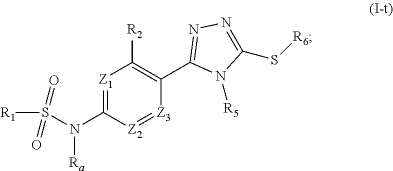
wherein, Z₁, Z₂, Z₃, Rₐ, R₁, R₂, R₅, and R₆ are as defined in claim 1.

29. A compound of any of claims 1 and 2, or a pharmaceutically acceptable salt thereof, having the formula: 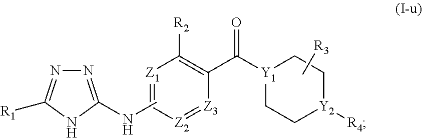
wherein, Z₁, Z₂, Z₃, Y₁, Y₂, R₁, R₂, R₃, and R₄ are as defined in claim 1.

30. A compound of any of claims 1 and 2, or a pharmaceutically acceptable salt thereof, having the formula: 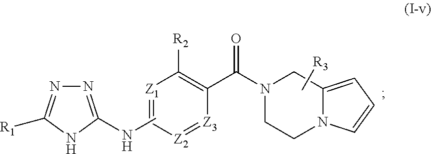
wherein, Z₁, Z₂, Z₃, R₁, R₂, and R₃ are as defined in claim 1.

31. A compound of any of claims 1 and 2, or a pharmaceutically acceptable salt thereof, having the formula: 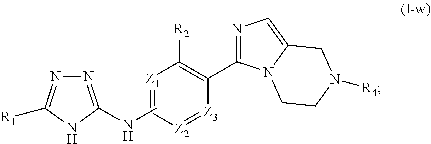
wherein, Z₁, Z₂, Z₃, R₁, R₂, and R₄ are as defined in claim 1.

32. A compound of any of claims 1 and 2, or a pharmaceutically acceptable salt thereof, having the formula: 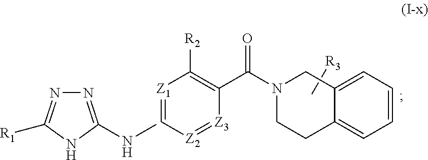
wherein, Z₁, Z₂, Z₃, R₁, R₂, and R₃ are as defined in claim 1.

33. A compound of any of claims 1 and 2, or a pharmaceutically acceptable salt thereof, having the formula: 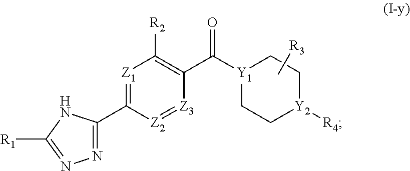
wherein, Z₁, Z₂, Z₃, Y₁, Y₂, R₁, R₂, R₃, and R₄ are as defined in claim 1.

34. A compound of any of claims 1 and 2, or a pharmaceutically acceptable salt thereof, having the formula: 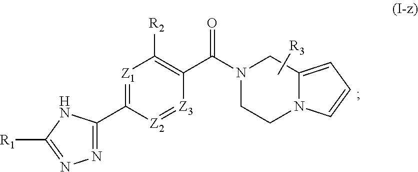
wherein, Z₁, Z₂, Z₃, R₁, R₂, and R₃ are as defined in claim 1.

35. A compound of any of claims 1 and 2, or a pharmaceutically acceptable salt thereof, having the formula: 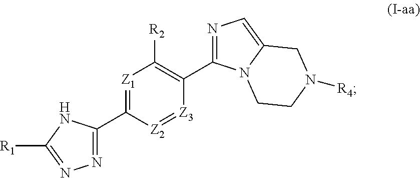
wherein, Z₁, Z₂, Z₃, R₁, R₂, and R₄ are as defined in claim 1.

36. A compound of any of claims 1 and 2, or a pharmaceutically acceptable salt thereof, having the formula: 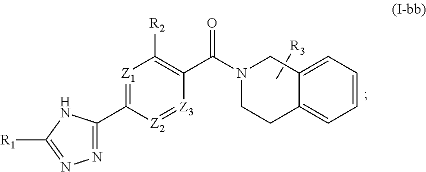
wherein, Z₁, Z₂, Z₃, R₁, R₂, and R₃ are as defined in claim 1.

37. A compound of any of claims 1 and 2, or a pharmaceutically acceptable salt thereof, having the formula: 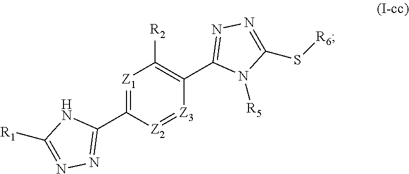
wherein, Z₁, Z₂, Z₃, R₁, R₂, R₅, and R₆ are as defined in claim 1.

38. A compound of any of claims 1 and 2, or a pharmaceutically acceptable salt thereof, having the formula: 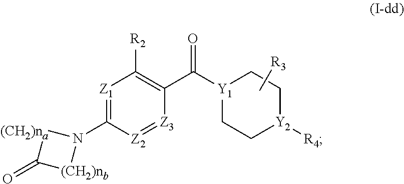
wherein, Z₁, Z₂, Z₃, Y₁, Y₂,R₂, R₃, R₄, nₐ, and n_{b}are as defined in claim 1.

39. A compound of any of claims 1 and 2, or a pharmaceutically acceptable salt thereof, having the formula: 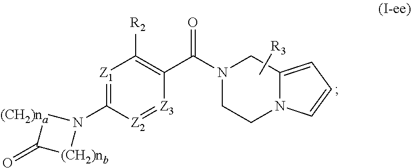
wherein, Z₁, Z₂, Z₃, R₂, R₃, nₐ, and n_{b}are as defined in claim 1.

40. A compound of any of claims 1 and 2, or a pharmaceutically acceptable salt thereof, having the formula: 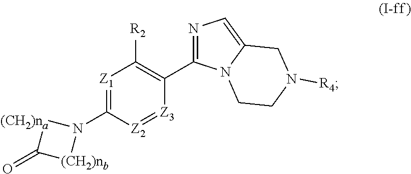
wherein, Z₁, Z₂, Z₃, R₂, R₄, nₐ, and n_{b}are as defined in claim 1.

41. A compound of any of claims 1 and 2, or a pharmaceutically acceptable salt thereof, having the formula: 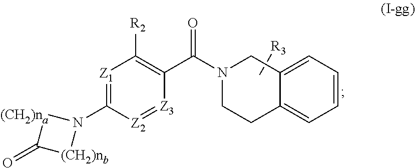
wherein, Z₁, Z₂, Z₃, R₂, R₃, nₐ, and n_{b}are as defined in claim 1.

42. A compound of any of claims 1 and 2, or a pharmaceutically acceptable salt thereof, having the formula: 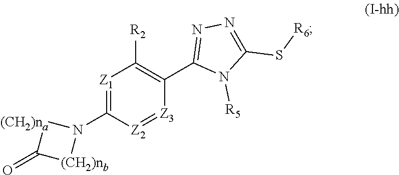
and
wherein, Z₁, Z₂, Z₃, R₂, R₅ , and R₆ are as defined in claim 1.

43. A compound of any of claims 1 and 3, or a pharmaceutically acceptable salt thereof, having the formula: 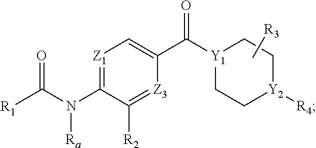
wherein, Z₁, Z₃, Y₁, Y₂, Rₐ, R₁, R₂, R₃, and R₄ are as defined in claim 1.

44. The compound of any of claims 1-42, or a pharmaceutically acceptable salt thereof, wherein Z₁, Z₂, and Z₃ are each C.

45. The compound of any of claims 1-42, or a pharmaceutically acceptable salt thereof, wherein Z₁, Z₂, and Z₃ are each N.

46. The compound of any of claims 1-42, or a pharmaceutically acceptable salt thereof, wherein Z₁ and Z₂ are each C.

47. The compound of any of claims 1-42, or a pharmaceutically acceptable salt thereof, wherein Z₁ and Z₃ are each C and Z₂ is N.

48. The compound of any of claims 1-43, or a pharmaceutically acceptable salt thereof, wherein Z₁ and Z₃ are each N and Z₂ is C.

49. The compound of any of claims 1-42, or a pharmaceutically acceptable salt thereof, wherein Z₁ and Z₂ are each N and Z₃ is C.

50. The compound of any of claims 1-42, or a pharmaceutically acceptable salt thereof, wherein Z2 and Z3 are each N and Z₁ is C.

51. The compound of any of claims 1-50, or a pharmaceutically acceptable salt thereof, wherein R₂ is selected from the group of: 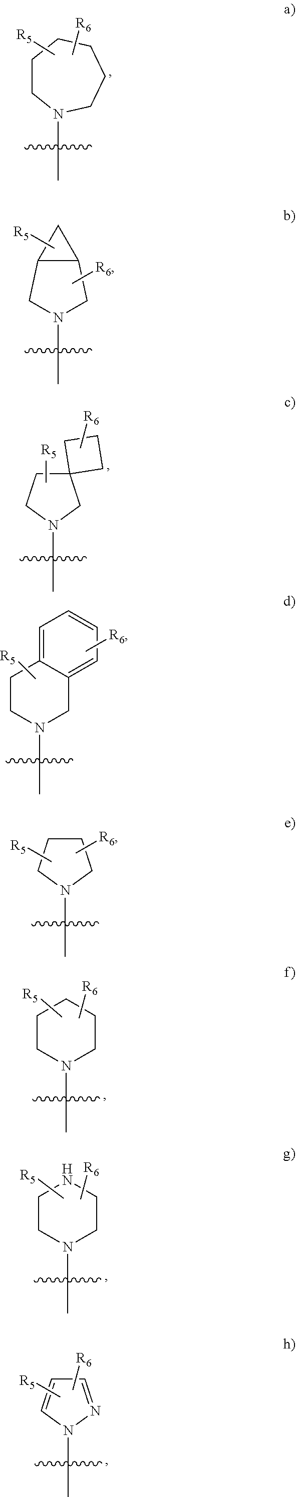 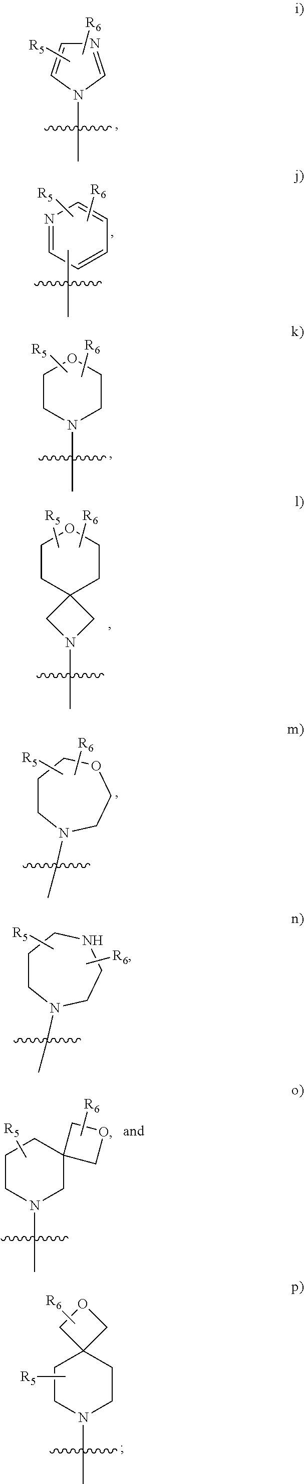
wherein R₅ and R₆ are each independently selected from hydrogen, halogen, C₁-C₆ alkyl, —O—C₁-C₆ alkyl, and —CF₃.

52. The compound of any of claims 1-51, or a pharmaceutically acceptable salt thereof, wherein R₂ is selected from the group of:

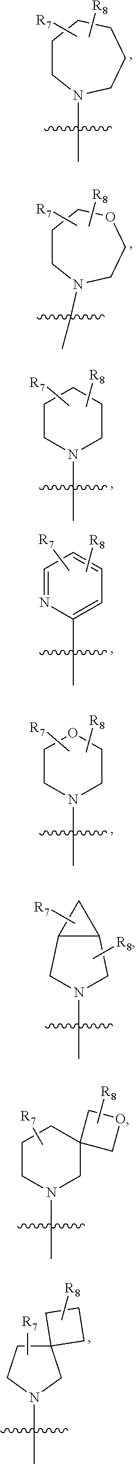

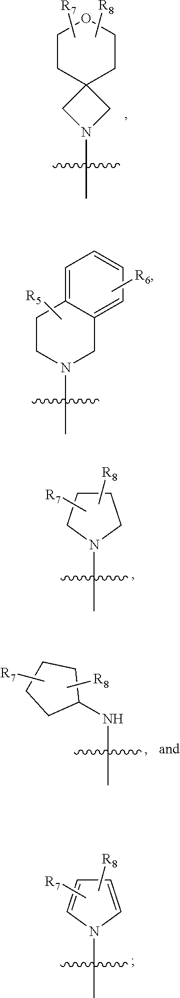

53. The compound of any of claims 1-52, or a pharmaceutically acceptable salt thereof, wherein R₂ is selected from the group of: 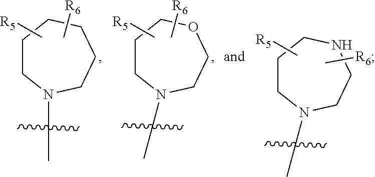
wherein R₅ and R₆ are each independently selected from hydrogen, halogen, C₁-C₆ alkyl, —O—C₁-C₆ alkyl, and —CF₃.

54. The compound of any of claims 1-52, or a pharmaceutically acceptable salt thereof, wherein R₂ is the group of: 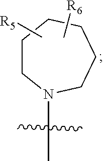
wherein R₅ and R₆ are each independently selected from hydrogen, halogen, C₁-C₆ alkyl, —O—C₁-C₆ alkyl, and —CF₃.

55. The compound of any of claims 1-52, or a pharmaceutically acceptable salt thereof, R₂ is selected from the group of: 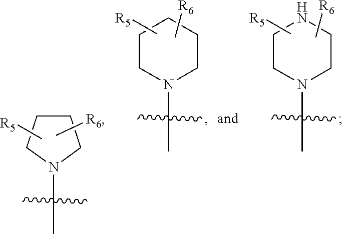
wherein R₅ and R₆ are each independently selected from hydrogen, halogen, C₁-C₆ alkyl, —O—C₁-C₆ alkyl, and —CF₃.

56. The compound of any of claims 1-55, or a pharmaceutically acceptable salt thereof, wherein R₅ and R₆ are each independently selected from the group of hydrogen, C₁-C₄ alkyl, and C₁-C₄ alkoxy.

57. The compound of any of claims 1-56, or a pharmaceutically acceptable salt thereof, wherein R₅ and R₆ are each independently selected from the group of hydrogen, C₁-C₃ alkyl, and C₁-C₃ alkoxy.

58. The compound of any of claims 1-57, or a pharmaceutically acceptable salt thereof, wherein R₃ is present 1-3 times, is present 2 times, or is present only 1 time; and OPTIONALLY either wherein R₃ is selected from the group of hydrogen, C₁-C₄ alkyl, C₃-C₆ cycloalkyl, —CF₃, —C₁-C₄ alkyl-OH, phenyl, benzyl, pyrazolyl, and thiophenyl; wherein the phenyl, pyrazolyl, and thiophenyl rings are substituted by 0, 1, 2, or 3 substituents selected from the group of halogen, OH, CF₃, C₁-C₄ alkyl, and C₁-C₄ alkoxy; or wherein R₃ is selected from the group of phenyl, benzyl, pyrazolyl, and thiophenyl, substituted by 0, 1, 2, or 3 substituents selected from the group of halogen, OH, CF3, C₁-C₄ alkyl, and C₁-C₄ alkoxy.

59. The compound of any of claims 1, 4, 9, 14, 19, 24, 29, 33, 38, and 43, or a pharmaceutically acceptable salt thereof, wherein Y₁ and Y₂ are each N.

60. The compound of any of claims 1, 4, 9, 14, 19, 24, 29, 33, 38, and 43, or a pharmaceutically acceptable salt thereof, wherein Y₁is N and Y₂ is C.

61. The compound of any of claims 1, 4, 9, 14, 19, 24, 29, 33, 38, and 43, or a pharmaceutically acceptable salt thereof, wherein Y₁is C and Y₂ is N.

62. The compound of any of claims 1-61, or a pharmaceutically acceptable salt thereof, wherein R₁ is C₁-C₆ alkyl substituted by 0, 1, 2, or 3 substituents selected from the group of halogen, OH, CF₃, and —O—C₁-C₃ alkyl.

63. The compound of any of claims 1-61, or a pharmaceutically acceptable salt thereof, wherein R₁ is C₁-C₄ alkyl substituted by 0, 1, 2, or 3 substituents selected from the group of halogen, OH, CF₃, and —O—C₁-C₃ alkyl.

64. The compound of any of claims 1-61, or a pharmaceutically acceptable salt thereof, wherein R₁ is selected from the group of —(CH₂)ₙ₁-C₃-C₆cycloalkyl, phenyl, and benzyl, wherein the rings of the —(CH₂)ₙ₁-C₃-C₆cycloalkyl, phenyl, and benzyl groups are substituted by 0, 1, 2, or 3 substituents selected from the group of halogen, OH, CF₃, and —O—C₁-C₃ alkyl.

65. The compound of any of claims 1, and 9-28, or a pharmaceutically acceptable salt thereof, wherein Rₐ is H.

66. The compound of any of claims 1 and 9-28, or a pharmaceutically acceptable salt thereof, wherein Rₐ is C₁-C₃ alkyl.

67. The compound of any of claims 1, 4-6, 9, 10, 12, 14, 15, 17, 19, 20, 22, 24, 25, 27, 29, 30, 32-34, 36, 38, 39, 41, and 43, or a pharmaceutically acceptable salt thereof, wherein R₃ is H.

68. The compound of any of claims 1, 4-6, 9, 10, 12, 14, 15, 17, 19, 20, 22, 24, 25, 27, 29, 30, 32-34, 36, 38, 39, 41, and 43, or a pharmaceutically acceptable salt thereof, wherein R₃ is C₁-C₃ alkyl.

69. The compound of any of claims 1, 4-6, 9, 10, 12, 14, 15, 17, 19, 20, 22, 24, 25, 27, 29, 30, 32-34, 36, 38, 39, 41, and 43, or a pharmaceutically acceptable salt thereof, wherein R₃ is selected from the group of CO₂H and —CO₂—(C₁-C₆ alkyl).

70. The compound of any of claims 1, 4-6, 9, 10, 12, 14, 15, 17, 19, 20, 22, 24, 25, 27, 29, 30, 32-34, 36, 38, 39, 41, and 43, or a pharmaceutically acceptable salt thereof, wherein R₃ is selected from the group of phenyl, benzyl, C₃-C₆ cycloalkyl, and —CH₂-C₃-C₆ cycloalkyl, the rings of each of the phenyl, benzyl, C₃-C₆ cycloalkyl, and —CH₂-C₃-C₆ cycloalkyl groups being substituted by 0, 1, 2, or 3 substituents selected from OH, halogen, and C₁-C₆ alkyl, wherein the C₁-C₆ alkyl group is further substituted by 0, 1, 2, 3, 4 or 5 substituents selected from the group of halogen, —CF₃, and OH.

71. The compound of any of claims 1, 4-6, 9, 10, 12, 14, 15, 17, 19, 20, 22, 24, 25, 27, 29, 30, 32-34, 36, 38, 39, 41, and 43, or a pharmaceutically acceptable salt thereof, wherein R₃ is selected from the group of a 5-membered or 6-membered heterocyclic ring containing 1, 2, or 3 ring heteroatoms independently selected from O, S, and N, the 5-membered or 6-membered heterocyclic ring being substituted by 0, 1, 2, or 3 substituents selected from OH, halogen, and C₁-C₆ alkyl, wherein the C₁-C₆ alkyl group is further substituted by 0, 1, 2, 3, 4 or 5 substituents selected from the group of halogen and OH.

72. The compound of any of claims 1 and 38-42, or a pharmaceutically acceptable salt thereof, wherein the sum of nₐ +n_{b}is 3.

73. The compound of any of claims 1 and 38-42, or a pharmaceutically acceptable salt thereof, wherein the sum of nₐ+n_{b}is 4.

74. The compound of any of claims 1, 4, 7, 9, 11,14, 16, 19, 21, 24, 26, 29, 31, 33, 35, 38, 40, and 43, or a pharmaceutically acceptable salt thereof, wherein R₄ is C₁-C₆ alkyl substituted by 0, 1, 2, or 3 substituents selected from the group of halogen, CF₃, OH, and —O—C₁-C₃ alkyl.

75. The compound of any of claims 1, 4, 7, 9, 11,14, 16, 19, 21, 24, 26, 29, 31, 33, 35, 38, 40, and 43, or a pharmaceutically acceptable salt thereof, wherein R₄ is C₁-C₄ alkyl substituted by 0, 1, 2, or 3 substituents selected from the group of halogen, CF₃, OH, and —O—C₁-C₃ alkyl.

76. The compound of any of claims 1, 4, 7, 9, 11,14, 16, 19, 21, 24, 26, 29, 31, 33, 35, 38, 40, and 43, or a pharmaceutically acceptable salt thereof, wherein R₄ is C₁-C₄ alkyl substituted by 0, 1, 2, or 3 halogen and OH.

77. The compound of any of claims 1, 4, 7, 9, 11,14, 16, 19, 21, 24, 26, 29, 31, 33, 35, 38, 40, and 43, or a pharmaceutically acceptable salt thereof, wherein R₄ is —(CH2)n2-C3-C6 cycloalkyl, wherein the cycloalkyl ring of the -(CH₂),₂-C₃-C₆ cycloalkyl group is substituted by 0, 1, 2, or 3 substituents selected from the group of halogen, CF₃, OH, and —O—C₁-C₃ alkyl: and n2 is an integer selected from the group of 0, 1, 2, and 3.

78. The compound of any of claims 1, 4, 7, 9, 11,14, 16, 19, 21, 24, 26, 29, 31, 33, 35, 38, 40, and 43, or a pharmaceutically acceptable salt thereof, wherein R₄ is selected from the group of phenyl and benzyl, with the rings of the phenyl and benzyl groups being substituted by 0, 1, 2, or 3 substituents selected from the group of halogen, CF₃, OH, and —O—C₁-C₃ alkyl.

79. The compound of any of claims 1, 4, 7, 9, 11,14, 16, 19, 21, 24, 26, 29, 31, 33, 35, 38, 40, and 43, or a pharmaceutically acceptable salt thereof, wherein R₄ is a heterocyclic or heteroaromatic ring having 3, 4, 5, 6, 7, 8, 9, or 10 ring atoms of which 1, 2, 3, or 4 ring atoms are selected from the group of N, O, and S; wherein the rings of the phenyl and benzyl groups are each independently substituted by 0, 1, 2, or 3 substituents selected from the group of halogen, CF₃, OH, and —O—C₁-C₃ alkyl.

80. The compound of any of claims 1, 4, 7, 9, 11,14, 16, 19, 21, 24, 26, 29, 31, 33, 35, 38, 40, and 43, or a pharmaceutically acceptable salt thereof, wherein Y₂ is C and EITHER R₄ is selected from the group of H, —O—C₁-C₆ alkyl, and —O—(CH₂),₂-C₃-C₆cycloalkyl; wherein the 0-C₁-C₆ alkyl group and the cycloalkyl ring of the —O—(CH₂)ₙ₂-C₃-C₆ cycloalkyl are each independently substituted by 0, 1, 2, or 3 substituents selected from the group of halogen, CF₃, OH, and —O—C₁-C₃ alkyl OR two R₄ form a C₃-C₆ cycloalkyl carbocycle or heterocycle containing 2-5 ring carbon atoms and 1 or 2 ring heteroatoms selected from the group of N and O.

81. The compound of claim 80, or a pharmaceutically acceptable salt thereof, wherein the —C₁-C₆ alkyl group and the cycloalkyl ring of the —O—(CH₂),₂-C₃-C₆ cycloalkyl are each independently substituted by 0, 1, 2, or 3 substituents selected from the group of halogen and OH.

82. A pharmaceutical composition comprising a pharmaceutically effective amount of a compound selected from any of claims 1-81, a compound selected from Examples 1-220, a compound of Formula (I′), or a pharmaceutically acceptable salt of any of the foregoing, and a pharmaceutically acceptable carrier or excipient.

83. The use of a compound selected from any of claims 1-81, a compound selected from Examples 1-220, a compound of Formula (I′), or a pharmaceutically acceptable salt of any of the foregoing, in the preparation of a medicament.

84. A compound selected from any of claims 1-81, a compound selected from Examples 1-220, a compound of Formula (I′), or a pharmaceutically acceptable salt of any of the foregoing, for use as a medicament.

85. A method of inhibiting the activity of a GPR39 protein in a subject, the method comprising administering to the subject in need thereof a pharmaceutically effective amount of a compound selected from any of claims 1-81, a compound selected from Examples 1-220, a compound of Formula (I′), or a pharmaceutically acceptable salt of any of the foregoing.

86. A method of treating hypertension in a human in need thereof, the method comprising administering to the human a pharmaceutically effective amount of a compound selected from any of claims 1-81, a compound selected from Examples 1-220, a compound of Formula (I′), or a pharmaceutically acceptable salt of any of the foregoing.

87. The method of claim 86, wherein the hypertension is primary hypertension.

88. The method of claim 86, wherein the hypertension is secondary hypertension.

89. The method of claim 86, wherein the hypertension is treatment-resistant or refractory hypertension.

90. The method of claim 86, wherein the hypertension is pulmonary hypertension.

91. A method of treating heart failure in a human, the method comprising administering to the human in need thereof a pharmaceutically effective amount of a compound selected from any of claims 1-81, a compound selected from Examples 1-220, a compound of Formula (I′), or a pharmaceutically acceptable salt of any of the foregoing.

92. A method of breast cancer in a human in need thereof, the method comprising administering to the human a pharmaceutically effective amount of a compound selected from any of claims 1-81, a compound selected from Examples 1-220, a compound of Formula (I′), or a pharmaceutically acceptable salt of any of the foregoing.

93. A method of gastric adenocarcinomas in a human in need thereof, the method comprising administering to the human a pharmaceutically effective amount of a compound selected from any of claims 1-81, a compound selected from Examples 1-220, a compound of Formula (I′), or a pharmaceutically acceptable salt of any of the foregoing.

94. A method of promoting or enhancing colon epithelial function and tight junction barrier integrity in a human, the method comprising administering to the human in need thereof a pharmaceutically effective amount of a compound selected from any of claims 1-81, a compound selected from Examples 1-220, a compound of Formula (I′), or a pharmaceutically acceptable salt of any of the foregoing.

95. A method of treating ulcerative colon disease in a human, the method comprising administering to the human in need thereof a pharmaceutically effective amount of a compound selected from any of claims 1-81, a compound selected from Examples 1-220, a compound of Formula (I′), or a pharmaceutically acceptable salt of any of the foregoing.

96. The method of claim 95, wherein the ulcerative colon disease is ulcerative colitis.

97. The method of claim 95, wherein the ulcerative colon disease is Crohn's Disease.

98. A method of treating Inflammatory Bowel Disease in a human, the method comprising administering to the human in need thereof a pharmaceutically effective amount of a compound selected from any of claims 1-81, a compound selected from Examples 1-220, a compound of Formula (I′), or a pharmaceutically acceptable salt of any of the foregoing.

99. A method of treating diarrhea in a human, the method comprising administering to the human in need thereof a pharmaceutically effective amount of a compound selected from any of claims 1-81, a compound selected from Examples 1-220, a compound of Formula (I′), or a pharmaceutically acceptable salt of any of the foregoing.

100. A method of enhancing the delivery of an anesthetic to a human experiencing microvascular complications, the method comprising administering to the human in need thereof a pharmaceutically effective amount of a compound selected from any of claims 1-81, a compound selected from Examples 1-220, a compound of Formula (I′), or a pharmaceutically acceptable salt of any of the foregoing, and a pharmaceutically effective amount of the anesthetic.

101. The method of claim 100, wherein the human is experiencing microvascular complications associated with or caused by prediabetes.

102. The method of claim 100, wherein the human is experiencing microvascular complications associated with or caused by diabetes.

103. A method of treating stroke in a human in need thereof, the method comprising administering to the human a pharmaceutically effective amount of a compound selected from any of claims 1-81, a compound selected from Examples 1-220, a compound of Formula (I′), or a pharmaceutically acceptable salt of any of the foregoing.

104. A kit comprising:
a) one or more compositions, each composition comprising a pharmaceutically effective amount of a compound selected from any of claims 1-81, a compound selected from Examples 1-220, a compound of Formula (I′), or a pharmaceutically acceptable salt of any of the foregoing, and a pharmaceutically acceptable carrier or excipient; and
b) instructions for administering the one or more compositions to a human in need thereof.

105. A compound of any one of Examples 1-34, 37-42, 44, 47, 48, 50, 51, and 53-220, or a pharmaceutically acceptable salt thereof.
